(19) United States
(12) Patent Application Publication
(10) Pub. No.: US 2016/0115540 A1
(43) Pub. Date: Apr. 28, 2016

(54) PRODRUGS AND DRUGS

(71) Applicant: Biogen MA Inc., Cambridge, MA (US)

(72) Inventors: Jason Fontenot, Jamaica Plain, MA (US); David Huss, Winchester, MA (US); Robert H. Scannevin, Hopkinton, MA (US); Kenneth Rhodes, Belmont, MA (US)

(21) Appl. No.: 14/892,839

(22) PCT Filed: May 21, 2014
(86) PCT No.: PCT/US14/38973

§ 371 Date: Nov. 20, 2015

Related U.S. Application Data
(60) Provisional application No. 61/825,938, filed on May 21, 2013.

(51) Int. Cl. C12Q 1/68; G01N 33/68; A61K 31/225

(52) U.S. Cl. CPC C12Q 1/6883; A61K 31/225; G01N 33/6893; G01N 2800/52; C12Q 2600/158; C12Q 2600/106; G01N 2800/285

(57) ABSTRACT

Methods and systems to evaluate a prodrug are provided.

### CROSS-REFERENCE TO RELATED APPLICATIONS

This application claims the benefit of U.S. Provisional Application No. 61/825,938, filed May 21, 2013, the entire contents of which are incorporated herein by reference.

### FIELD OF THE INVENTION

The invention relates, inter alia, to the use and activity of prodrugs and their drugs, e.g., dimethyl fumarate (DMF) and monomethyl fumarate (MMF), e.g., in the treatment of multiple sclerosis (MS) and other disorders.

### BACKGROUND OF THE INVENTION

The relationship between a drug and its metabolite and their contribution to overall pharmacologic effect is often poorly understood.

Tecfidera® (BG-12, dimethyl fumarate, DMF) is a methyl ester of fumaric acid. Tecfidera® is an oral therapeutic approved in the U.S. for relapsing multiple sclerosis (MS). MS is an inflammatory disease of the brain and spinal cord characterized by recurrent foci of inflammation that lead to destruction of the myelin sheath. In many areas, nerve fibers are also damaged.

Preclinical studies indicate that activation of the nuclear factor (erythroid-derived 2)-like 2(Nrf2) pathway is thought to be involved in the clinical effects of Tecfidera®. In vivo, DMF is rapidly metabolized to monomethyl fumarate (MMF), and both compounds are pharmacologically active. In vitro, DMF and MMF share some common effects, but also have divergent pharmacological properties.

Given the destructive effects of inflammatory MS lesions and the distinct effects of therapies such as DMF and MMF, the need exists for evaluating or monitoring a subject undergoing an MS therapy, or identifying a subject that would benefit from an MS therapy.

### SUMMARY OF THE INVENTION

The present invention provides, at least in part, methods, devices, reaction mixtures and kits for evaluating, identifying, and/or treating a subject, e.g., a subject having multiple sclerosis (MS) (e.g., a subject with relapsing MS). In certain embodiments, responsiveness of a subject to a treatment (e.g., an MS therapy that includes dimethyl fumarate) is evaluated by detecting a differential expression (e.g., level and/or expression), of a gene (e.g., a gene or a gene product) in response to a treatment that includes DMF and/or monomethyl fumarate (MMF). Applicants have identified both specific and common responses to DMF treatment and to MMF treatment in selected tissues and blood, e.g., whole blood, in a subject. Without being bound by theory, the specific responses, e.g., transcriptional signatures, induced by DMF and MMF indicate that not all the DMF in vivo effects are mediated through MMF, thus suggesting that DMF can directly mediate unique biological responses, not captured by MMF alone. Thus, the invention can, therefore, be used, for example: To evaluate responsiveness to, or monitor, a therapy or treatment that includes DMF; identify a subject as likely to benefit from a therapy or treatment that includes DMF; stratify a subject or a patient populations (e.g., stratify a subject or patients as being likely or unlikely to respond to a therapy or treatment that includes DMF); and/or more effectively monitor, treat a disorder, e.g., MS, or prevent worsening of disease and/or relapse. Many of the methods, devices, reaction mixtures and other inventions provided herein are described for use with DMF and its active metabolite MMF. However, it should be understood that the methods, devices, reaction mixtures and other inventions can be used with, or apply generically to, dialkyl fumarate prodrugs, e.g., as shown in Formula A below, and other prodrugs, e.g., as shown in Formulas I-X, and their active metabolites (e.g., MMF).

Accordingly, in one aspect, the invention features a method of evaluating, monitoring, stratifying, or treating, a subject. The method includes:

a) acquiring a value for the expression of a gene (e.g., a gene or a gene product), wherein said gene is chosen from one, two or all of:
i) a dimethyl fumarate (DMF)-differentially expressed gene,
ii) a monomethyl fumarate (MMF)-differentially expressed gene, or
iii) a DMF/MMF-differentially expressed gene;

b) responsive to said value, performing one, two or all of:
i) classifying said subject,
ii) selecting or identifying said subject for treatment with DMF, or with a treatment other than DMF, or
iii) administering DMF, or a treatment other than DMF, to said subject,

provided that the method comprises one of treating the subject, directly acquiring the value, or directly acquiring a sample from which the value is acquired.

In a related aspect, the invention features a method of evaluating, or monitoring, a treatment (e.g., an MS treatment, e.g., an MS treatment with a DMF) in a subject (e.g., a subject, a patient, a patient group or population, having MS, or at risk for developing MS). The method includes:

administering to the subject, e.g., a subject in need of treatment (e.g., an MS treatment), a DMF;

acquiring from said subject a value for the expression of a gene (e.g., a gene or a gene product), wherein said gene is chosen from one, two or all of:
i) a dimethyl fumarate (DMF)-differentially expressed gene,
ii) a monomethyl fumarate (MMF)-differentially expressed gene, or
iii) a DMF/MMF-differentially expressed gene,
   wherein a change in (i) or (ii) is indicative of a differential response to DMF or MMF, respectively, and a change in (iii) is indicative of a response to both DMF and MMF.

In certain embodiments, the method further comprises, responsive to said value, treating, selecting and/or altering one or more of: the course of the treatment (e.g., MS treatment), the dosing of the treatment (e.g., MS treatment), the schedule or time course of the treatment (e.g., MS treatment), or administration of a second, alternative treatment (e.g., a treatment other than DMF).

In another related aspect, the invention features a method of treating a subject, e.g., a subject having, or at risk of having, MS. The method includes:

administering to the subject a DMF in an amount sufficient to treat MS, provided that the subject is identified for treatment with the DMF on the basis of a value for the expression of a gene, wherein said gene is chosen from one, two or all of:
i) a dimethyl fumarate (DMF)-differentially expressed gene,
ii) a monomethyl fumarate (MMF)-differentially expressed gene, or
iii) a DMF/MMF-differentially expressed gene.

Additional embodiments or features of any of the above aspects are as follows:

### Acquiring a Value

In certain embodiments, the method comprises acquiring a value for the expression of a plurality, e.g., 2, 3, 4, 5, 6, 7, 8, 9, 10 or more, genes, and, optionally, any step responsive thereto can be responsive to one, some, or all, of the acquired values.

In certain embodiments, the gene used in acquiring the value is chosen from one, two or all of: a DMF-differentially expressed gene, an MMF-differentially expressed gene, or a gene expressed in response to both DMF and MMF (e.g., a DMF/MMF-differentially expressed gene).

In one embodiment, the value for expression of the gene includes a value for a transcriptional parameter, e.g., the level of an mRNA encoded by the gene.

In another embodiment, the value for expression of the gene includes a value for a translational parameter, e.g., the level of a protein encoded by the gene.

In certain embodiments, the method includes acquiring a value for the expression of a plurality of genes. In certain embodiments, said plurality includes two, three, four or more of:

a) a plurality, e.g., 2, 3, 4, 5, 6, 7, 8, 9 or 10, or more, DMF-differentially expressed genes;

b) a plurality, e.g., 2, 3, 4, 5, 6, 7, 8, 9 or 10, or more, MMF-differentially expressed genes;

c) a DMF-differentially expressed gene and an MMF-differentially expressed gene;

d) a DMF-differentially expressed gene and a gene that is both DMF-differentially expressed and MMF-differentially expressed; and

e) an MMF-differentially expressed gene and a gene that is both DMF-differentially expressed and MMF-differentially expressed.

### Blood Genes

In certain embodiments, the value for expression of the gene acquired is from blood, e.g., whole blood (e.g., a gene expressed in blood or a blood sample).

In one embodiment, the value for expression of the gene includes a value for a transcriptional parameter, e.g., the level of an mRNA encoded by the gene, in blood, e.g., whole blood. In certain embodiments, the gene is selected from one or more of the genes in Table 1 or Table 9. In embodiments, the gene is a gene from Table 9 that shows differential expression as measured by mRNA levels. In one embodiment, the differential expression is detected prior to or after (e.g., 2, 3, 5, 7, 10, 12, 15 or 24 hours after) administration of a treatment (e.g., a DMF or an MMF). In one embodiment, the gene is chosen from one, two, three, four or all of: Granzyme A (Gzma), Natural cytotoxicity triggering receptor 1 (Ncr1), Killer cell lectin-like receptor subfamily C member 1 (Klrc1), Killer cell lectin-like receptor subfamily B member 1B (Klrb1b), or Killer cell lectin-like receptor family E member 1 (Klre1). In one embodiment, the gene is chosen from one, two, three or all of: Granzyme A (Gzma), Natural cytotoxicity triggering receptor 1 (Ncr1), Killer cell lectin-like receptor subfamily C member 1 (Klrc1), or Killer cell lectin-like receptor subfamily B member 1B (Klrb1b). In an embodiment, the gene is an NFkB activated gene, e.g., a gene chosen from one, two, three, or all of: Fc Fragment Of IgG, High Affinity Ia, Receptor (FCGR1A), Suppression Of Tumorigenicity 18 (ST18), Chemokine (C-C motif) ligand 3-like 1 (CCL3L1), or Vascular cell adhesion protein 1 (VCAM1). In an embodiment, the gene is an IL-2 activated gene, e.g., a gene chosen from one, two, three or all of: chemokine (C-C motif) receptor 3 (CCR3), Killer cell lectin-like receptor subfamily B member 1C (Klrb1c), Natural cytotoxicity triggering receptor 1 (Ncr1), or Chemokine (C-C motif) ligand 3-like 1 (CCL3L1). In an embodiment, the gene is decidual protein induced by progesterone (DEPP). In an embodiment, the gene is zinc finger and BTB domain containing 16 (Zbtb16), or an isoform thereof. In an embodiment, the gene is selected from 1, 2, 3, 4, 5, 6, 7, 8 or all of FCGR1A, ST18, CCL3L1, VCAM1, CCR3, Klrb1c, Ncr1, DEPP, or Zbtb16, or an isoform thereof.

In an embodiment, the method, e.g., method described herein, includes acquiring a value for the expression of FCGR1A. In an embodiment, the method, e.g., method described herein, includes acquiring a value for the expression of ST18. In an embodiment, the method, e.g., method described herein, includes acquiring a value for the expression of CCL3L1. In an embodiment, the method, e.g., method described herein, includes acquiring a value for the expression of VCAM1. In an embodiment, the method, e.g., method described herein, includes acquiring a value for the expression of, CCR3. In an embodiment, the method, e.g., method described herein, includes acquiring a value for the expression of Klrb1c. In an embodiment, the method, e.g., method described herein, includes acquiring a value for the expression of Ncr1. In an embodiment, the method, e.g., method described herein, includes acquiring a value for the expression of DEPP. In an embodiment, the method, e.g., method described herein, includes acquiring a value for the expression of Zbtb16.

In one embodiment, the value for expression of the gene comprises a value for a transcriptional parameter, e.g., the level of an mRNA encoded by the gene, in blood, for 1, 2, 3, 4, or all of, Gzma, Ncr1, Klrc1, Klrb1b, and Klre1. In one embodiment, the value for expression of the gene comprises a value for a transcriptional parameter, e.g., the level of an mRNA encoded by the gene, in blood, for 1, 2, 3, or all of, Gzma, Ncr1, Klrc1, and Klrb1b. In an embodiment, the value for expression of the gene comprises a value for a transcriptional parameter, e.g., the level of an mRNA encoded by the gene, in blood, for 1, 2, 3, or all of, FCGR1A, ST18, CCL3L1, or VCAM1. In an embodiment, the value for expression of the gene comprises a value for a transcriptional parameter, e.g., the level of an mRNA encoded by the gene, in blood, for 1, 2, 3, or all of CCR3, Klrb1c, Ncr1, or CCL3L1. In an embodiment, the value for expression of the gene comprises a value for a transcriptional parameter, e.g., the level of an mRNA encoded by the gene, in blood, for DEPP. In an embodiment, the value for expression of the gene comprises a value for a transcriptional parameter, e.g., the level of an mRNA encoded by the gene, in blood, for Zbtb16, or an isoform thereof. In an embodiment, In an embodiment, the value for expression of the gene comprises a value for a transcriptional parameter, e.g., the level of an mRNA encoded by the gene, in blood, for 1, 2, 3, 4, 5, 6, 7, 8 or all of FCGR1A, ST18, CCL3L1, VCAM1, CCR3, Klrb1c, Ncr1, DEPP, or Zbtb16, or an isoform thereof.

In other embodiments, the value for expression of the gene comprises a value for a translational parameter, e.g., the level of a protein encoded by the gene, in blood, e.g., whole blood. In certain embodiments, the gene is selected from one or more of the genes in Table 1 or Table 9. In embodiments, the gene is a gene from Table 9 that shows differential expression as measured by protein levels. In one embodiment, the differential expression is detected prior to or after (e.g., 2, 3, 5, 7, 10, 12, 15 or 24 hours after) administration of a treatment (e.g., a DMF or an MMF). In certain embodiments, the gene is chosen from one, two, three, or all of: Killer cell lectin-like receptor subfamily C member 1 (Klrc1), Killer cell lectin-like receptor subfamily B member 1B (Klrb1b), NKKG2d (Klrk1), or Natural killer cells (CD94) (Klrd1). In an embodiment, the gene is an NFkB activated gene, e.g., a gene chosen from one, two, three, or all of: Fc Fragment Of IgG, High Affinity Ia, Receptor (FCGR1A), Suppression Of Tumorigenicity 18 (ST18), Chemokine (C-C motif) ligand 3-like 1 (CCL3L1), or Vascular cell adhesion protein 1 (VCAM1). In an embodiment, the gene is an IL-2 activated gene, e.g., a gene chosen from one, two, three or all of: chemokine (C-C motif) receptor 3 (CCR3), Killer cell lectin-like receptor subfamily B member 1C (Klrb1c), Natural cytotoxicity triggering receptor 1 (Ncr1), or Chemokine (C-C motif) ligand 3-like 1 (CCL3L1). In an embodiment, the gene is decidual protein induced by progesterone (DEPP). In an embodiment, the gene is zinc finger and BTB domain containing 16 (Zbtb16), or an isoform thereof. In an embodiment, the gene is chosen from 1, 2, 3, 4, 5, 6, 7, 8 or all of FCGR1A, ST18, CCL3L1, VCAM1, CCR3, Klrb1c, Ncr1, DEPP, or Zbtb16, or an isoform thereof.

In one embodiment, a value for expression of the gene comprises a value for a translational parameter, e.g., the level of a protein encoded by the gene, in blood, for 1, 2, 3, or all of, Klrc1, Klrb1b, Klrk1, and Klrd1. In an embodiment, a value for expression of the gene comprises a value for a translational parameter, e.g., the level of a protein encoded by the gene, in blood, for 1, 2, 3 or all of FCGR1A, ST18, CCL3L1, or VCAM1. In an embodiment, a value for expression of the gene comprises a value for a translational parameter, e.g., the level of a protein encoded by the gene, in blood, for 1, 2, 3, or all of CCR3, Klrb1c, Ncr1, or CCL3L1. In an embodiment, a value for expression of the gene comprises a value for a translational parameter, e.g., the level of a protein encoded by the gene, in blood, for DEPP. In an embodiment, a value for expression of the gene comprises a value for a translational parameter, e.g., the level of a protein encoded by the gene, in blood, for Zbtb16, or an isoform thereof. In an embodiment, a value for expression of the gene comprises a value for a translational parameter, e.g., the level of a protein encoded by the gene, in blood, for 1, 2, 3, 4, 5, 6, 7, 8 or all of FCGR1A, ST18, CCL3L1, VCAM1, CCR3, Klrb1c, Ncr1, DEPP, or Zbtb16, or an isoform thereof.

In certain embodiments, the value for expression of the gene is for a blood sample, or a blood derived sample, e.g., serum or plasma, or an NK-cell containing fraction, from the subject.

In certain embodiments, the blood comprises, greater than background levels, e.g., therapeutic levels, of DMF, MMF, or both.

### Other Tissues

In certain embodiments, the value for expression of the gene is for a tissue, e.g., a tissue selected from cortical tissue, hippocampus, striatum, jejunum, kidney, liver, or spleen. In an embodiment, the value for expression of the gene is for spinal cord, brain, or combination thereof. In certain embodiments, the value for expression of the gene is for cerebrospinal fluid. In an embodiment, the value for expression of the gene is for lymph node, spleen, or combination thereof.

In certain embodiments, said gene is selected from the genes in Table 2, Table 3, Table 4, Table 5a, Table 5b, Table 6, Table 7, Table 8, Appendix A, Appendix B, Appendix C, Appendix D, or Appendix E.

In one embodiment, the value for expression of the gene includes a value for a transcriptional parameter, e.g., the level of an mRNA encoded by the gene, in tissue, e.g., whole tissue. In certain embodiments, the gene is selected from one or more of the genes in Table 1 or Table 9. In embodiments, the gene is a gene from Table 9 that shows differential expression as measured by mRNA levels. In one embodiment, the differential expression is detected prior to or after (e.g., 2, 3, 5, 7, 10, 12, 15 or 24 hours after) administration of a treatment (e.g., a DMF or an MMF). In one embodiment, the gene is chosen from one, two, three, four or all of: Granzyme A (Gzma), Natural cytotoxicity triggering receptor 1 (Ncr1), Killer cell lectin-like receptor subfamily C member 1 (Klrc1), Killer cell lectin-like receptor subfamily B member 1B (Klrb1b), or Killer cell lectin-like receptor family E member 1 (Klre1). In one embodiment, the gene is chosen from one, two, three or all of: Granzyme A (Gzma), Natural cytotoxicity triggering receptor 1 (Ncr1), Killer cell lectin-like receptor subfamily C member 1 (Klrc1), or Killer cell lectin-like receptor subfamily B member 1B (Klrb1b). In an embodiment, the gene is an NFkB activated gene, e.g., a gene chosen from one, two, three, or all of: Fc Fragment Of IgG, High Affinity Ia, Receptor (FCGR1A), Suppression Of Tumorigenicity 18 (ST18), Chemokine (C-C motif) ligand 3-like 1 (CCL3L1), or Vascular cell adhesion protein 1 (VCAM1). In an embodiment, the gene is an IL-2 activated gene, e.g., a gene chosen from one, two, three or all of: chemokine (C-C motif) receptor 3 (CCR3), Killer cell lectin-like receptor subfamily B member 1C (Klrb1c), Natural cytotoxicity triggering receptor 1 (Ncr1), or Chemokine (C-C motif) ligand 3-like 1 (CCL3L1). In an embodiment, the gene is decidual protein induced by progesterone (DEPP). In an embodiment, the gene is zinc finger and BTB domain containing 16 (Zbtb16), or an isoform thereof. In one embodiment, the gene is chosen from 1, 2, 3, 4, 5, 6, 7, 8 or all of FCGR1A, ST18, CCL3L1, VCAM1, CCR3, Klrb1c, Ncr1, DEPP, or Zbtb16, or an isoform thereof

In one embodiment, the value for expression of the gene comprises a value for a transcriptional parameter, e.g., the level of an mRNA encoded by the gene, in tissue, for 1, 2, 3, 4, or all of, Gzma, Ncr1, Klrc1, Klrb1b, and Klre1. In one embodiment, the value for expression of the gene comprises a value for a transcriptional parameter, e.g., the level of an mRNA encoded by the gene, in tissue, for 1, 2, 3, or all of, Gzma, Ncr1, Klrc1, and Klrb1b. In an embodiment, the value for expression of the gene comprises a value for a transcriptional parameter, e.g., the level of an mRNA encoded by the gene, in tissue, for 1, 2, 3, or all of, FCGR1A, ST18, CCL3L1, or VCAM1. In an embodiment, the value for expression of the gene comprises a value for a transcriptional parameter, e.g., the level of an mRNA encoded by the gene, in tissue, for 1, 2, 3, or all of CCR3, Klrb1c, Ncr1, or CCL3L1. In an embodiment, the value for expression of the gene comprises a value for a transcriptional parameter, e.g., the level of an mRNA encoded by the gene, in tissue, for DEPP. In an embodiment, the value for expression of the gene comprises a value for a transcriptional parameter, e.g., the level of an mRNA encoded by the gene, in tissue, for Zbtb16, or an isoform thereof. In one embodiment, the value for expression of the gene comprises a value for a transcriptional parameter, e.g., the level of an mRNA encoded by the gene, in tissue, for 1, 2, 3, 4, 5, 6, 7, 8 or all of FCGR1A, ST18, CCL3L1, VCAM1, CCR3, Klrb1c, Ncr1, DEPP, or Zbtb16, or an isoform thereof.

In other embodiments, the value for expression of the gene comprises a value for a translational parameter, e.g., the level of a protein encoded by the gene, in tissue, e.g., whole tissue. In certain embodiments, the gene is selected from one or more of the genes in Table 1 or Table 9. In embodiments, the gene is a gene from Table 9 that shows differential expression as measured by protein levels. In one embodiment, the differential expression is detected prior to or after (e.g., 2, 3, 5, 7, 10, 12, 15 or 24 hours after) administration of a treatment (e.g., a DMF or an MMF). In certain embodiments, the gene is chosen from one, two, three, or all of: Killer cell lectin-like receptor subfamily C member 1 (Klrc1), Killer cell lectin-like receptor subfamily B member 1B (Klrb1b), NKKG2d (Klrk1), or Natural killer cells (CD94) (Klrd1). In an embodiment, the gene is an NFkB activated gene, e.g., a gene chosen from one, two, three, or all of: Fc Fragment Of IgG, High Affinity Ia, Receptor (FCGR1A), Suppression Of Tumorigenicity 18 (ST18), Chemokine (C-C motif) ligand 3-like 1 (CCL3L1), or Vascular cell adhesion protein 1 (VCAM1). In an embodiment, the gene is an IL-2 activated gene, e.g., a gene chosen from one, two, three or all of: chemokine (C-C motif) receptor 3 (CCR3), Killer cell lectin-like receptor subfamily B member 1C (Klrb1c), Natural cytotoxicity triggering receptor 1 (Ncr1), or Chemokine (C-C motif) ligand 3-like 1 (CCL3L1). In an embodiment, the gene is decidual protein induced by progesterone (DEPP). In an embodiment, the gene is zinc finger and BTB domain containing 16 (Zbtb16), or an isoform thereof. In one embodiment, the gene is chosen from 1, 2, 3, 4, 5, 6, 7, 8 or all of FCGR1A, ST18, CCL3L1, VCAM1, CCR3, Klrb1c, Ncr1, DEPP, or Zbtb16, or an isoform thereof.

In one embodiment, a value for expression of the gene comprises a value for a translational parameter, e.g., the level of a protein encoded by the gene, in tissue, for 1, 2, 3, or all of, Klrc1, Klrb1b, Klrk1, and Klrd1. In an embodiment, a value for expression of the gene comprises a value for a translational parameter, e.g., the level of a protein encoded by the gene, in tissue, for 1, 2, 3 or all of FCGR1A, ST18, CCL3L1, or VCAM1. In an embodiment, a value for expression of the gene comprises a value for a translational parameter, e.g., the level of a protein encoded by the gene, in tissue, for 1, 2, 3, or all of CCR3, Klrb1c, Ncr1, or CCL3L1. In an embodiment, a value for expression of the gene comprises a value for a translational parameter, e.g., the level of a protein encoded by the gene, in tissue, for DEPP. In an embodiment, a value for expression of the gene comprises a value for a translational parameter, e.g., the level of a protein encoded by the gene, in tissue, for Zbtb16, or an isoform thereof. In an embodiment, a value for expression of the gene comprises a value for a translational parameter, e.g., the level of a protein encoded by the gene, in tissue, for 1, 2, 3, 4, 5, 6, 7, 8 or all of FCGR1A, ST18, CCL3L1, VCAM1, CCR3, Klrb1c, Ncr1, DEPP, or Zbtb16, or an isoform thereof.

### Timing of Evaluation or Administration

In some embodiments, the value is acquired at one or more of the following periods: prior to beginning of treatment; during the treatment; or after the treatment has been administered. In embodiments, the treatment is an MS treatment (e.g., a treatment that includes a DMF).

In certain embodiments, the subject has been administered the treatment, e.g., the DMF, e.g., prior to, at the time of, or after, acquiring the value. In one embodiment, the value is acquired after (e.g., 2, 3, 5, 7, 10, 12, 15 or 24 hours after) administration of a treatment (e.g., a DMF).

In one embodiment, the methods described herein include the step of comparing the value (e.g., level) of one or more genes described herein to a specified parameter (e.g., a reference value or sample; a sample obtained from a healthy subject; a sample obtained from the subject at different treatment intervals). For example, a sample can be analyzed at any stage of treatment, but preferably, prior to, during, or after terminating, administration of the therapy, e.g., the MS therapy.

In certain embodiments, the methods include the step of detecting the level of one or more genes in the subject, prior to, or after, administering the therapy (e.g., MS therapy), to the subject. In an embodiment, a change in gene expression indicates that the subject from whom the sample was obtained is responding to the therapy, e.g., the MS therapy.

### Tissue/Sample

In certain embodiments, a tissue (e.g., cerebrospinal fluid) or blood (e.g., a tissue or blood sample) of the subject, e.g., the peripheral blood, comprises, greater than background levels, e.g., therapeutic levels, of DMF, MMF, or both, e.g., prior to, or at the time of, acquiring the value.

In certain embodiments, the sample is chosen from a non-cellular body fluid; or a cellular or tissue fraction. In one embodiment, the non-cellular fraction is chosen from blood, e.g., whole blood, plasma or serum. In other embodiments, the cellular fraction comprises one or more of: T cells, B cells or myeloid cells. For example, the cellular fraction can include one or more of: natural killer (NK) cells, peripheral blood mononuclear cells (PBMC), CD8+ T cells, or Regulatory T cells. In an embodiment, the sample is cerebrospinal fluid.

In certain embodiments, the methods described herein, further includes the step of acquiring the sample, e.g., a biological sample, from the subject.

A sample can include any material obtained and/or derived from a biological sample, including a polypeptide, and nucleic acid (e.g., genomic DNA, cDNA, RNA) purified or processed from the sample.

### Subjects

For any of the methods, devices or kits disclosed herein, the subject treated, or the subject from which the value or sample is acquired, is a subject having, or at risk of having MS at any stage of treatment. In certain embodiments, the MS patient is chosen from a patient having one or more of: Benign MS, relapsing MS, e.g., relapsing-remitting MS (RRMS) (e.g., quiescent RRMS, active RRMS), primary progressive MS, or secondary progressive MS. In other embodiments, the subject has MS-like symptoms, such as those having clinically isolated syndrome (CIS) or clinically defined MS (CDMS). In one embodiment, the subject is an MS patient (e.g., a patient with relapsing MS) prior to administration of an MS therapy described herein (e.g., prior to administration of a DMF). In another embodiment, the subject is an MS patient (e.g., a relapsing MS patient) after administration of an MS therapy described herein (e.g., a DMF). In other embodiments, the subject is an MS patient after administration of the MS therapy for one, two, five, ten, twenty, twenty four hours; one week, two weeks, one month, two months, three months, four months, six months, one year or more.

In one embodiment, the subject has a relapsing form of MS, e.g., RRMS.

### Treatment/Other Therapies

Alternatively, or in combination with the methods described herein, the invention features a method of treating a subject having one or more symptoms associated with MS. In one embodiment, the subject is identified as responding or not responding to a therapy, using the methods, devices, or kits described herein.

In an embodiment the method comprises treating the subject with DMF, MMF, or a combination thereof.

In certain embodiments, the treatment includes reducing, retarding or preventing, a relapse, or the worsening of a disability, in the MS patients.

In one embodiment, the method includes administering to a subject (e.g., a subject described herein) a therapy for MS (e.g., a DMF), in an amount sufficient to reduce one or more symptoms associated with MS.

In embodiments where a first therapy (e.g., the DMF therapy) is not detectably effective, an alternative or other MS therapy can be chosen. Exemplary other therapies include, but are not limited to, an IFN-b agent (e.g., an IFN-b 1a molecule or an IFN-b 1b molecule, including analogues and derivatives thereof (e.g., pegylated variants thereof)). In one embodiment, the other MS therapy includes an IFN-b 1a agent (e.g., Avonex®, Rebif®). In another embodiment, the other MS therapy includes an INF-b 1b agent (e.g., Betaseron®, Betaferon®). In other embodiments, the other MS therapy includes a polymer of four amino acids found in myelin basic protein, e.g., a polymer of glutamic acid, lysine, alanine and tyrosine (e.g., glatiramer (Copaxone®)); an antibody or fragment thereof against alpha-4 integrin (e.g., natalizumab (Tysabri®)); an anthracenedione molecule (e.g., mitoxantrone (Novantrone®)); fingolimod (FTY720; Gilenya®); Daclizumab; alemtuzumab (Lemtrada®)); or an anti-LINGO-1 antibody. In certain embodiments, the methods include the use of one or more symptom management therapies, such as antidepressants, analgesics, anti-tremor agents, among others.

### Detection Methods

In certain embodiments, the gene or gene product detected is, e.g., nucleic acid, cDNA, RNA (e.g., mRNA), or a polypeptide.

A nucleic acid can be detected, or the level determined, by any means of nucleic acid detection, or detection of the expression level of the nucleic acids, including but not limited to, nucleic acid hybridization assay, amplification-based assays (e.g., polymerase chain reaction), sequencing, and/or in situ hybridization.

In certain embodiments, a probe is a nucleic acid that specifically hybridizes with a transcription product of the gene or genes. In other embodiments, the detection includes amplification of a transcription product of the gene or genes. In other embodiments, the detection includes reverse transcription and amplification of a transcription product of the gene or genes.

In other embodiments, a translation product of the gene or genes, e.g., a polypeptide, is detected. The polypeptide can be detected, or the level determined, by any means of polypeptide detection, or detection of the expression level of the polypeptides. For example, the polypeptide can be detected using a probe or reagent which specifically binds with the polypeptides. In another embodiment, the reagent is selected from the group consisting of an antibody, an antibody derivative, and an antibody fragment, e.g., a labeled antibody (e.g., a fluorescent or a radioactive label), or fragment thereof, that specifically binds with a translation product of the gene or genes. In one embodiment, the polypeptide is detected using antibody-based detection techniques, such as enzyme-based immunoabsorbent assay, immunofluorescence cell sorting (FACS), immunohistochemistry, immunofluorescence (IF), antigen retrieval and/or microarray detection methods. Polypeptide detection methods can be performed in any other assay format, including but not limited to, ELISA, RIA, and mass spectrometry.

In certain embodiments, the probe is an antibody. In one embodiment, the method of detection includes a sandwich-based detection, e.g., ELISA based sandwich assay detection, of a translation product of the gene or genes.

### Other Embodiments

The methods of the invention can further include the step of monitoring the subject, e.g., for a change (e.g., an increase or decrease) in one or more of: levels of one or more MS biomarkers; the rate of appearance of new lesions, e.g., in an MRI scan; the appearance of new disease-related symptoms; a change in EDSS score; a change in quality of life; or any other parameter related to clinical outcome. The subject can be monitored in one or more of the following periods: prior to beginning of treatment; during the treatment; or after the treatment has been administered. Monitoring can be used to evaluate the need for further treatment with the same MS therapy, or for additional MS treatment. Generally, a decrease in one or more of the parameters described above is indicative of the improved condition of the subject.

In certain embodiments, the methods described herein further include: performing a neurological examination, evaluating the subject's status on the Expanded Disability Status Scale (EDSS), or detecting the subject's lesion status as assessed using an MRI.

### Devices

In another aspect, the invention features a device comprising:

one, or a plurality of, e.g., 2, 3, 4, 5, 6, 7, 8, 9 or 10, or more, probes, each probe being specific for a product, e.g., a translational product or transcriptional product, of a gene selected independently from:

i) a dimethyl fumarate (DMF)-differentially expressed gene,

ii) a monomethyl fumarate (MMF)-differentially expressed gene, or

iii) a DMF/MMF-differentially expressed gene.

In one embodiment, the device includes one, or a plurality of, e.g., 2, 3, 4, 5, 6, 7, 8, 9 or 10, or more, probes, each probe being specific for a product, e.g., a translational product or transcriptional product, of a dimethyl fumarate (DMF)-differentially expressed gene.

In one embodiment, the probe or probes of the device are specific for a gene or genes selected from the genes in Table 9. In an embodiment, the probe or probes of the device are specific for a gene or genes in Appendix A, Appendix B, Appendix C, Appendix D or Appendix E.

In other embodiments, the probe or probes of the device are specific for a gene or genes selected from the genes in Table 9 that shows differential expression as measured by mRNA levels.

In yet other embodiments, the device includes a probe specific for a transcriptional product of 1, 2, 3, 4, or all of, Gzma, Ncr1, Klrc1, Klrb1b, and Klre1. In yet other embodiments, the device includes a probe specific for a transcriptional product of 1, 2, 3, or all of, Gzma, Ncr1, Klrc1, and Klrb1b. In yet other embodiments, the device includes a probe specific for a transcriptional product of 1, 2, 3, or all of, FCGR1A, ST18, CCL3L1, or VCAM1. In yet other embodiments, the device includes a probe specific for a transcriptional product of 1, 2, 3, or all of CCR3, Klrb1c, Ncr1, or CCL3L1. In yet other embodiments, the device includes a probe specific for a transcriptional product of DEPP. In yet other embodiments, the device includes a probe specific for a transcriptional product of Zbtb16, or an isoform thereof. In yet other embodiments, the device includes a probe specific for a transcriptional product of 1, 2, 3, 4, 5, 6, 7, 8 or all of FCGR1A, ST18, CCL3L1, VCAM1, CCR3, Klrb1c, Ncr1, DEPP, or Zbtb16, or an isoform thereof.

In other embodiments, the device includes a probe specific for a gene or genes from the genes in Table 9 that shows differential expression as measured by protein levels.

In other embodiments, the device includes a probe specific for a translational product of 1, 2, 3, or all of, Klrc1, Klrb1b, Klrk1, and Klrd1. In other embodiments, the device includes a probe specific for a translational product of 1, 2, 3, or all of, FCGR1A, ST18, CCL3L1, or VCAM1. In yet other embodiments, the device includes a probe specific for a translational product of 1, 2, 3, or all of CCR3, Klrb1c, Ncr1, or CCL3L1. In yet other embodiments, the device includes a probe specific for a translational product of DEPP. In yet other embodiments, the device includes a probe specific for a translational product of Zbtb16, or an isoform thereof. In other embodiments, the device includes a probe specific for a translational product of 1, 2, 3, 4, 5, 6, 7, 8 or all of FCGR1A, ST18, CCL3L1, VCAM1, CCR3, Klrb1c, Ncr1, DEPP, or Zbtb16, or an isoform thereof

In one embodiment, the device further comprises a sample, e.g., a sample as described herein. In one embodiment, the sample is from a subject having an autoimmune disorder, e.g., MS, relapsing MS. In other embodiments, the sample is from a subject that has been administered DMF. In yet other embodiments, the sample is from a tissue of the subject, e.g., the peripheral blood, which comprises greater than background levels, e.g., therapeutic levels, of DMF, MMF, or both.

In other embodiments, the device further comprises a sample, e.g., a blood sample, or a substance derived from blood, e.g., serum, or an NK-cell containing fraction.

In yet other embodiments, the probe or probes of the device are specific for a gene or genes that are selected independently from the genes in Table 2, Table 3, Table 4, Table 5a, Table 5b, Table 6, Table 7, Table 8, Appendix A, Appendix B, Appendix C, Appendix D, or Appendix E.

In other embodiments, the probe is a nucleic acid that specifically hybridizes with a transcription product of the gene or genes.

In embodiments, the device is configured to allow amplification of a transcription product of the gene or genes.

In other embodiments, the device is configured to allow reverse transcription and amplification of a transcription product of the gene or genes.

In other embodiments, a probe is an antibody, e.g., a labeled antibody, or fragment thereof, that specifically binds with a translation product of the gene or genes.

In other embodiments, the device is configured to allow sandwich-based detection, e.g., ELISA based sandwich assay detection, of a translation product of the gene or genes.

In yet other embodiments, the device has less than 10, 25, 50, 100, 200, 250, 300, or 500 probes specific for products, e.g., a translational product or transcriptional product, of genes that are not

i) a dimethyl fumarate (DMF)-differentially expressed gene,

ii) a monomethyl fumarate (MMF)-differentially expressed gene, or

iii) a DMF/MMF-differentially expressed gene.

In one embodiment, the device has less than 10, 25, 50, 100, 200, 250, 300, or 500 probes specific for products, e.g., a translational product or transcriptional product, of genes that are not a dimethyl fumarate (DMF)-differentially expressed gene.

In yet other embodiments, the device has less than 10, 25, 50, 100, 200, 250, 300, or 500 probes specific for products, e.g., a translational product or transcriptional product, of genes that are not listed in Table 9.

In other embodiments, the device has at least 10, 20, 30, 40, 50, 60, 70, 80, or 90% of the probes of the device are specific for a product, e.g., a translational product or transcriptional product, of:

i) a dimethyl fumarate (DMF)-differentially expressed gene,

ii) a monomethyl fumarate (MMF)-differentially expressed gene, or

iii) a DMF/MMF-differentially expressed gene.

In other embodiments, the device has at least 10, 20, 30, 40, 50, 60, 70, 80, or 90% of the probes of the device are specific for a product, e.g., a translational product or transcriptional product, of a dimethyl fumarate (DMF)-differentially expressed gene.

In other embodiments, the device has at least 10, 20, 30, 40, 50, 60, 70, 80, or 90% of the probes of the device are specific for a product, e.g., a translational product or transcriptional product, of a gene listed in Table 9.

In certain embodiments, the probe or probes are disposed on a surface of the device.

In another aspect, the invention features a method of using a device described herein. The method includes:

providing a device described herein;

contacting the device with a sample described herein,

thereby using the device.

In one embodiment, the method includes a step of capturing a signal, e.g., an electronic, or visual signal, to evaluate the sample.

### Reaction Mixtures

In an aspect, the invention features a reaction mixture comprising:

a sample from a tissue of a subject, e.g., the peripheral blood, e.g., tissue which comprises greater than background levels, e.g., therapeutic levels, of DMF, MMF, or a prodrug of MMF, or a combination thereof; and

one, or a plurality of, probes each probe being specific for a product, e.g., a translational product or transcriptional product, of a gene described herein,

wherein the reaction mixture includes less than 10, 25, 50, 100, 200, 250, 300, or 500 probes specific for products, e.g., a translational product or transcriptional product, of genes other than the gene described herein.

In another aspect, the invention features, a reaction mixture comprising:

a sample; and

one, or a plurality of, e.g., 2, 3, 4, 5, 6, 7, 8, 9 or 10, or more, probes, each probe being specific for a product, e.g., a translational product or transcriptional product, of a gene selected independently from:

i) a dimethyl fumarate (DMF)-differentially expressed gene,

ii) a monomethyl fumarate (MMF)-differentially expressed gene, or

iii) a DMF/MMF-differentially expressed gene.

In an embodiment the reaction mixture comprises one, or a plurality of, e.g., 2, 3, 4, 5, 6, 7, 8, 9 or 10, or more, probes, each probe being specific for a product, e.g., a translational product or transcriptional product, of a dimethyl fumarate (DMF)-differentially expressed gene.

In another embodiment, the probe or probes are specific for a gene or genes selected from the genes in Table 9.

In another embodiment, the probe or probes are specific for a gene or genes selected from the genes in Table 9 that shows differential expression as measured by mRNA levels.

In one embodiment, the reaction mixture comprises probes specific for a transcriptional product of 1, 2, 3, 4, or all of, Gzma, Ncr1, Klrc1, Klrb1b, and Klre1. In one embodiment, the reaction mixture comprises probes specific for a transcriptional product of 1, 2, 3, or all of, Gzma, Ncr1, Klrc1, and Klrb1b. In one embodiment, the reaction mixture comprises probes specific for a transcriptional product of 1, 2, 3, or all of, FCGR1A, ST18, CCL3L1, or VCAM1. In one embodiment, the reaction mixture comprises probes specific for a transcriptional product of 1, 2, 3, or all of CCR3, Klrb1c, Ncr1, or CCL3L1. In one embodiment, the reaction mixture comprises probes specific for a transcriptional product of DEPP. In one embodiment, the reaction mixture comprises probes specific for a transcriptional product of Zbtb16, or an isoform thereof. In one embodiment, the reaction mixture comprises probes specific for a transcriptional product of 1, 2, 3, 4, 5, 6, 7, 8 or all of FCGR1A, ST18, CCL3L1, VCAM1, CCR3, Klrb1c, Ncr1, DEPP, or Zbtb16, or an isoform thereof.

In another embodiment, the probe or probes are specific for a gene or genes from the genes in Table 9 that shows differential expression as measured by protein levels.

In other embodiments, the reaction mixture comprises probes specific for a translational product of 1, 2, 3, or all of, Klrc1, Klrb1b, Klrk1, and Klrd1. In one embodiment, the reaction mixture comprises probes specific for a translational product of 1, 2, 3, or all of, FCGR1A, ST18, CCL3L1, or VCAM1. In one embodiment, the reaction mixture comprises probes specific for a translational product of 1, 2, 3, or all of CCR3, Klrb1c, Ncr1, or CCL3L1. In one embodiment, the reaction mixture comprises probes specific for a translational product of DEPP. In one embodiment, the reaction mixture comprises probes specific for a translational product of Zbtb16, or an isoform thereof. In one embodiment, the reaction mixture comprises probes specific for a translational product of 1, 2, 3, 4, 5, 6, 7, 8 or all of FCGR1A, ST18, CCL3L1, VCAM1, CCR3, Klrb1c, Ncr1, DEPP, or Zbtb16, or an isoform thereof.

In an embodiment, said sample is from a subject having an autoimmune disorder, e.g., MS, e.g., relapsing MS.

In one embodiment, said sample is from a subject that has been administered DMF.

In an embodiment, said sample is from a tissue of the subject, e.g., the peripheral blood, which comprises greater than background levels, e.g., therapeutic levels, of DMF, MMF, or both.

In an embodiment, said sample comprises blood, or a substance derived from blood, e.g., serum, or an NK-cell containing fraction.

In other embodiments, the probe or probes are specific for a gene or genes that are selected independently from the genes in Table 2, Table 3, Table 4, Table 5a, Table 5b, Table 6, Table 7, Table 8, Appendix A, Appendix B, Appendix C, Appendix D, or Appendix E.

In an embodiment, a probe is a nucleic acid that specifically hybridizes with a transcription product of the gene or genes.

In an embodiment, the reaction mixture further comprises reagents to allow for amplification of a transcription product of the gene or genes.

In an embodiment, the reaction mixture further comprises reagents to allow for reverse transcription and amplification of a transcription product of the gene or genes.

In an embodiment, a probe is an antibody, e.g., a labeled antibody, or fragment thereof, that specifically binds with a translation product of the gene or genes.

In an embodiment, the reaction mixture comprises reagents to allow sandwich-based detection, e.g., ELISA based sandwich assay detection, of a translation product of the gene or genes.

In other embodiments, the reaction mixture has less than 10, 25, 50, 100, 200, 250, 300, or 500 probes specific for products, e.g., a translational product or transcriptional product, of genes that are not

i) a dimethyl fumarate (DMF)-differentially expressed gene,

ii) a monomethyl fumarate (MMF)-differentially expressed gene, or

iii) a DMF/MMF-differentially expressed gene.

In other embodiments, the reaction mixture has less than 10, 25, 50, 100, 200, 250, 300, or 500 probes specific for products, e.g., a translational product or transcriptional product, of genes that are not a dimethyl fumarate (DMF)-differentially expressed gene.

In other embodiments, the reaction mixture has less than 10, 25, 50, 100, 200, 250, 300, or 500 probes specific for products, e.g., a translational product or transcriptional product, of genes that are not listed in Table 9.

In other embodiments, the reaction mixture has at least 10, 20, 30, 40, 50, 60, 70, 80, or 90% of the probes of the device are specific for a product, e.g., a translational product or transcriptional product, of:

i) a dimethyl fumarate (DMF)-differentially expressed gene,

ii) a monomethyl fumarate (MMF)-differentially expressed gene, or

iii) a DMF/MMF-differentially expressed gene.

In other embodiments, at least 10, 20, 30, 40, 50, 60, 70, 80, or 90% of the probes are specific for a product, e.g., a translational product or transcriptional product, of a dimethyl fumarate (DMF)-differentially expressed gene.

In other embodiments of the reaction mixture at least 10, 20, 30, 40, 50, 60, 70, 80, or 90% of the probes of the device are specific for a product, e.g., a translational product or transcriptional product, of a gene listed in Table 9.

The reaction mixture of can comprise a surface on which the probe or probes are disposed.

In another aspect, the invention features a method of making a reaction mixture comprising:

providing the a sample described herein;

contacting the sample with one or a plurality of probes described herein, or with a device described herein,

thereby making a reaction mixture.

In embodiments, the method of making includes capturing a signal, e.g., an electronic, or visual signal, to evaluate the sample.

### Kits

In another aspect, the invention features kits for evaluating a sample, e.g., a sample from an MS patient, to detect or determine the level of one or more genes as described herein. The kit includes a means for detection of (e.g., a reagent that specifically detects) one or more genes as described herein. In certain embodiments, the kit includes an MS therapy. In one another embodiment, the kit comprises an antibody, an antibody derivative, or an antibody fragment to a protein produce of the gene. In one embodiment, the kit includes an antibody-based detection technique, such as immunofluorescence cell sorting (FACS), immunohistochemistry, antigen retrieval and/or microarray detection reagents. In one embodiment, at least one of the reagents in the kit is an antibody that binds to a gene product (optionally) with a detectable label (e.g., a fluorescent or a radioactive label). In certain embodiments, the kit is an ELISA or an immunohistochemistry (IHC) assay for detection of the gene.

The methods, devices, reaction mixtures, kits, and other inventions described herein can further include providing or generating, and/or transmitting information, e.g., a report, containing data of the evaluation or treatment determined by the methods, assays, and/or kits as described herein. The information can be transmitted to a report-receiving party or entity (e.g., a patient, a health care provider, a diagnostic provider, and/or a regulatory agency, e.g., the FDA), or otherwise submitting information about the methods, assays and kits disclosed herein to another party. The method can relate to compliance with a regulatory requirement, e.g., a pre- or post approval requirement of a regulatory agency, e.g., the FDA. In one embodiment, the report-receiving party or entity can determine if a predetermined requirement or reference value is met by the data, and, optionally, a response from the report-receiving entity or party is received, e.g., by a physician, patient, diagnostic provider.

In a related aspect, the invention features a method of evaluating, or monitoring, a prodrug, in a subject, e.g., a human or a non-human mammal. The method includes:

administering the prodrug to the subject;

acquiring from said subject a value for the expression of a gene (e.g., a gene or a gene product), wherein said gene is chosen from one, two or all of:
i) a prodrug-differentially expressed gene,
ii) a drug-differentially expressed gene, or
iii) a prodrug/drug-differentially expressed gene,
   wherein a change in (i) or (ii) is indicative of a differential response to prodrug or drug, respectively, and a change in (iii) is indicative of a response to both prodrug and drug.

In certain embodiments, the method further comprises comparing the value with a reference value.

In an embodiment the drug is DMF and the metabolite is MMF

In an embodiment the drug metabolite is MMF and the drug or prodrug is a compound of Formula I:

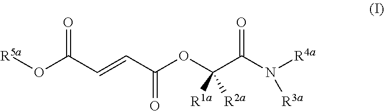

or a pharmaceutically acceptable salt thereof, wherein
R^{1a} and R^{2a} are independently chosen from hydrogen, C₁₋₆ alkyl, and substituted C₁₋₆ alkyl;
R^{3a} and R^{4a} are independently chosen from hydrogen, C₁₋₆ alkyl, substituted C₁₋₆ alkyl, C₁₋₆ heteroalkyl, substituted C₁₋₆ heteroalkyl, C₄₋₁₂ cycloalkylalkyl, substituted C₄₋₁₂ cycloalkylalkyl, C₇₋₁₂ arylalkyl, and substituted C₇₋₁₂ arylalkyl; or R^{3a} and R^{4a} together with the nitrogen to which they are bonded form a ring chosen from a C₅₋₁₀ heteroaryl, substituted C₅₋₁₀ heteroaryl, C₅₋₁₀ heterocycloalkyl, and substituted C₅₋₁₀ heterocycloalkyl; and
R^{5a} is chosen from methyl, ethyl, and C₃₋₆ alkyl;
wherein each substituent group is independently chosen from halogen, —OH, —CN, —CF₃, ═O, —NO₂, benzyl, —C(O)NR^{11a}₂, —R^{11a}, —OR^{11a}, —C(O)R^{11a}, —COOR^{11a}, and —NR^{11a}₂ wherein each R^{11a} is independently chosen from hydrogen and C₁₋₄ alkyl;
with the proviso that when R^{5a} is ethyl; then R^{3a} and R^{4a} are independently chosen from hydrogen, C₁₋₆ alkyl, and substituted C₁₋₆ alkyl.

In certain embodiments of a compound of Formula (I), each substituent group is independently chosen from halogen, —OH, —CN, —CF₃, —R^{11a}, —OR^{11a}, and —NR^{11a}₂ wherein each R^{11a} is independently chosen from hydrogen and C₁₋₄ alkyl. In certain embodiments, each substituent group is independently chosen from —OH, and —COOH.

In certain embodiments of a compound of Formula (I), each substituent group is independently chosen from ═O, C₁₋₄ alkyl, and —COOR^{11a} wherein R^{11a} is chosen from hydrogen and C₁₋₄ alkyl.

In certain embodiments of a compound of Formula (I), each of R^{1a} and R^{2a} is hydrogen.

In certain embodiments of a compound of Formula (I), one of R^{1a} and R^{2a} is hydrogen and the other of R^{1a} and R^{2a} is C₁₋₄ alkyl.

In certain embodiments of a compound of Formula (I), one of R^{1a} and R^{2a} is hydrogen and the other of R^{1a} and R^{2a} is chosen from methyl, ethyl, n-propyl, isopropyl, n-butyl, isobutyl, sec-butyl, and tert-butyl.

In certain embodiments of a compound of Formula (I), one of R^{1a} and R^{2a} is hydrogen and the other of R^{1a} and R^{2a} is methyl.

In certain embodiments of a compound of Formula (I), R^{3a} and R^{4a} are independently chosen from hydrogen and C₁₋₆ alkyl.

In certain embodiments of a compound of Formula (I), R^{3a} and R^{4a} are independently chosen from hydrogen and C₁₋₄ alkyl.

In certain embodiments of a compound of Formula (I), R^{3a} and R^{4a} are independently chosen from hydrogen, methyl, and ethyl.

In certain embodiments of a compound of Formula (I), each of R^{3a} and R^{4a} is hydrogen; in certain embodiments, each of R^{3a} and R^{4a} is methyl; and in certain embodiments, each of R^{3a} and R^{4a} is ethyl.

In certain embodiments of a compound of Formula (I), R^{3a} is hydrogen; and R^{4a} is chosen from C₁₋₄ alkyl, substituted C₁₋₄ alkyl wherein the substituent group is chosen from ═O, —OR^{11a}, —COOR^{11a}, and —NR^{11a}₂, wherein each R^{11a} is independently chosen form hydrogen and C₁₋₄ alkyl.

In certain embodiments of a compound of Formula (I), R^{3a} is hydrogen; and R^{4a} is chosen from C₁₋₄ alkyl, benzyl, 2-methoxyethyl, carboxymethyl, carboxypropyl, 1,2,4-thiadoxolyl, methoxy, 2-methoxycarbonyl, 2-oxo(1,3-oxazolidinyl), 2-(methylethoxy)ethyl, 2-ethoxyethyl, (tert-butyloxycarbonyl)methyl, (ethoxycarbonyl)methyl, carboxymethyl, (methylethyl)oxycarbonylmethyl, and ethoxycarbonylmethyl.

In certain embodiments of a compound of Formula (I), R^{3a} and R^{4a} together with the nitrogen to which they are bonded form a ring chosen from a C₅₋₆ heterocycloalkyl, substituted C₅₋₆ heterocycloalkyl, C₅₋₆ heteroaryl, and substituted C₅₋₆ heteroaryl ring. In certain embodiments of a compound of Formula (I), R^{3a} and R^{4a} together with the nitrogen to which they are bonded form a ring chosen from a C₅ heterocycloalkyl, substituted C₅ heterocycloalkyl, C₅ heteroaryl, and substituted C₅ heteroaryl ring. In certain embodiments of a compound of Formula (I), R^{3a} and R^{4a} together with the nitrogen to which they are bonded form a ring chosen from a C₆ heterocycloalkyl, substituted C₆ heterocycloalkyl, C₆ heteroaryl, and substituted C₆ heteroaryl ring. In certain embodiments of a compound of Formula (I), R^{3a} and R^{4a} together with the nitrogen to which they are bonded form a ring chosen from piperazine, 1,3-oxazolidinyl, pyrrolidine, and morpholine ring.

In certain embodiments of a compound of Formula (I), R^{3a} and R^{4a} together with the nitrogen to which they are bonded form a C₅₋₁₀ heterocycloalkyl ring.

In certain embodiments of a compound of Formula (I), R^{5a} is methyl.

In certain embodiments of a compound of Formula (I), R^{5a} is ethyl.

In certain embodiments of a compound of Formula (I), R^{5a} is C₃₋₆ alkyl.

In certain embodiments of a compound of Formula (I), R^{5a} is chosen from methyl, n-propyl, isopropyl, n-butyl, sec-butyl, isobutyl, and tert-butyl.

In certain embodiments of a compound of Formula (I), R^{5a} is chosen from methyl, ethyl, n-propyl, isopropyl, n-butyl, sec-butyl, isobutyl, and tert-butyl.

In certain embodiments of a compound of Formula (I), one of R^{1a} and R^{2a} is hydrogen and the other of R^{1a} and R^{2a} is C₁₋₆ alkyl; R^{3a} is hydrogen; R^{4a} is chosen from hydrogen, C₁₋₆ alkyl, and benzyl.

In certain embodiments of a compound of Formula (I), one of R^{1a} and R^{2a} is hydrogen and the other of R^{1a} and R^{2a} is C₁₋₆ alkyl; R^{3a} is hydrogen; R^{4a} is chosen from hydrogen, C₁₋₆ alkyl, and benzyl; and R^{5a} is methyl.

In certain embodiments of a compound of Formula (I), one of R^{1a} and R^{2a} is hydrogen and the other of R^{1a} and R^{2a} is chosen from hydrogen and C₁₋₆ alkyl; and each of R^{3a} and R^{4a} is C₁₋₆ alkyl.

In certain embodiments of a compound of Formula (I), one of R^{1a} and R^{2a} is hydrogen and the other of R^{1a} and R^{2a} is chosen from hydrogen and C₁₋₆ alkyl; each of R^{3a} and R^{4a} is C₁₋₆ alkyl; and R^{5a} is methyl. In certain embodiments of a compound of Formula (I), each of R^{1a} and R^{2a} is hydrogen; each of R^{3a} and R^{4a} is C₁₋₆ alkyl; and R^{5a} is methyl.

In certain embodiments of a compound of Formula (I), one of R^{1a} and R^{2a} is hydrogen and the other of R^{1a} and R^{2a} is chosen from hydrogen and C₁₋₄ alkyl; R^{3a} is hydrogen; R^{4a} is chosen from C₁₋₄ alkyl, substituted C₁₋₄ alkyl wherein the substituent group is chosen from ═O, —OR^{11a}, —COOR^{11a}, and —NR^{11a}₂, wherein each R^{11a} is independently chosen form hydrogen and C₁₋₄ alkyl; and R^{5a} is methyl. In certain embodiments of a compound of Formula (I), one of R^{1a} and R^{2a} is hydrogen and the other of R^{1a} and R^{2a} is methyl; R^{3a} is hydrogen; R^{4a} is chosen from C₁₋₄ alkyl, substituted C₁₋₄ alkyl wherein the substituent group is chosen from ═O, —OR^{11a}, —COOR^{11a}, and

—NR^{11a}₂, wherein each R^{11a} is independently chosen form hydrogen and C₁₋₄ alkyl; and R^{5a} is methyl. In certain embodiments of a compound of Formula (I), each of R^{1a} and R^{2a} is hydrogen; R^{3a} is hydrogen; R^{4a} is chosen from C₁₋₄ alkyl, substituted C₁₋₄ alkyl wherein the substituent group is chosen from ═O, —OR^{11a}, —COOR^{11a}, and —NR^{11a}₂, wherein each R^{11a} is independently chosen form hydrogen and C₁₋₄ alkyl; and R^{5a} is methyl.

In certain embodiments of a compound of Formula (I), R^{3a} and R^{4a} together with the nitrogen to which they are bonded form a C₅₋₁₀ heterocycloalkyl ring.

In certain embodiments of a compound of Formula (I), one of R^{1a} and R^{2a} is hydrogen and the other of R^{1a} and R^{2a} is chosen from hydrogen and C₁₋₆ alkyl; R^{3a} and R^{4a} together with the nitrogen to which they are bonded form a ring chosen from a C₅₋₆ heterocycloalkyl, substituted C₅₋₆ heterocycloalkyl, C₅₋₆ heteroaryl, and substituted C₅₋₆ heteroaryl ring; and R^{5a} is methyl. In certain embodiments of a compound of Formula (I), one of R^{1a} and R^{2a} is hydrogen and the other of R^{1a} and R^{2a} is methyl; R^{3a} and R^{4a} together with the nitrogen to which they are bonded form a ring chosen from a C₅₋₆ heterocycloalkyl, substituted C₅₋₆ heterocycloalkyl, C₅₋₆ heteroaryl, and substituted C₅₋₆ heteroaryl ring; and R^{5a} is methyl. In certain embodiments of a compound of Formula (I), each of R^{1a} and R^{2a} is hydrogen; R^{3a} and R^{4a} together with the nitrogen to which they are bonded form a ring chosen from a C₅₋₆ heterocycloalkyl, substituted C₅₋₆ heterocycloalkyl, C₅₋₆ heteroaryl, and substituted C₅₋₆ heteroaryl ring; and R^{5a} is methyl.

In certain embodiments of a compound of Formula (I), one of R^{1a} and R^{2a} is hydrogen and the other of R^{1a} and R^{2a} is chosen from hydrogen and C₁₋₆ alkyl; and R^{3a} and R^{4a} together with the nitrogen to which they are bonded form a ring chosen from morpholine, piperazine, and N-substituted piperazine.

In certain embodiments of a compound of Formula (I), one of R^{1a} and R^{2a} is hydrogen and the other of R^{1a} and R^{2a} is chosen from hydrogen and C₁₋₆ alkyl; R^{3a} and R^{4a} together with the nitrogen to which they are bonded form a ring chosen from morpholine, piperazine, and N-substituted piperazine; and R^{5a} is methyl.

In certain embodiments of a compound of Formula (I), R^{5a} is not methyl.

In certain embodiments of a compound of Formula (I), R^{1a} is hydrogen, and in certain embodiments, R^{2a} is hydrogen.

In certain embodiments of a compound of Formula (I), the compound is chosen from: (N,N-diethylcarbamoyl)methyl methyl(2E)but-2-ene-1,4-dioate; methyl[N-benzylcarbamoyl]methyl(2E)but-2-ene-1,4-dioate; methyl 2-morpholin-4-yl-2-oxoethyl(2E)but-2-ene-1,4-dioate; (N-butylcarbamoyl)methyl methyl(2E)but-2-ene-1,4-dioate; [N-(2-methoxyethyl)carbamoyl]methyl methyl(2E)but-2-ene-1,4-dioate; 2-{2-[(2E)-3-(methoxycarbonyl)prop-2-enoyloxy]acetylamino}acetic acid; 4-{2-[(2E)-3-(methoxycarbonyl)prop-2-enoyloxy]acetylamino}butanoic acid; methyl(N-(1,3,4-thiadiazol-2-yl)carbamoyl)methyl(2E)but-2ene-1,4-dioate; (N,N-dimethylcarbamoyl)methyl methyl(2E)but-2-ene-1,4-dioate; (N-methoxy-N-methylcarbamoyl)methyl methyl(2E)but-2-ene-1,4-dioate; bis-(2-methoxyethylamino)carbamoyl]methyl methyl(2E)but-2-ene-1,4-dioate; [N-(methoxycarbonyl)carbamoyl]methyl methyl(2E)but-2ene-1,4-dioate; 4-{2-[(2E)-3-(methoxycarbonyl)prop-2-enoyloxy]acetylamino}butanoic acid, sodium salt; methyl 2-oxo-2-piperazinylethyl(2E)but-2-ene-1,4-dioate; methyl 2-oxo-2-(2-oxo(1,3-oxazolidin-3-yl)ethyl(2E)but-2ene-1,4-dioate; {N-[2-(dimethylamino)ethyl]carbamoy}methyl methyl(2E)but-2ene-1,4 dioate; methyl 2-(4-methylpiperazinyl)-2-oxoethyl(2E)but-2-ene-1,4-dioate; methyl {N-[(propylamino)carbonyl]carbamoyl}methyl(2E)but-2ene-1,4-dioate; 2-(4-acetylpiperazinyl)-2-oxoethyl methyl(2E)but-2ene-1,4-dioate; {N,N-bis[2-(methylethoxy)ethyl]carbamoy}methyl methyl(2E)but-2-ene-1,4-dioate; methyl 2-(4-benzylpiperazinyl)-2-oxoethyl(2E)but-2-ene-1,4-dioate; [N,N-bis(2-ethoxyethyl)carbamoyl]methyl methyl(2E)but-2-ene-1,4-dioate; 2-{(2S)-2-[(tert-butyl)oxycarbonyl]pyrrolidinyl}-2-oxoethyl methyl(2E)but-2ene-1,4-dioate; 1-{2-{(2E)-3-(methoxycarbonyl)prop-2-enoyloxy]acetyl}(2S)pyrrolidine-2-carboxylic acid; (N-{[tert-butyl)oxycarbonyl]methyl}-N-methylcarbamoyl)methyl methyl(2E)but-2ene1,4-dioate; {N-(ethoxycarbonyl)methyl]-N-methylcarbamoyl}methyl methyl(2E)but-2-ene-1,4-dioate; methyl 1-methyl-2-morpholin-4-yl-2-oxoethyl(2E)but-2-ene-1,4-dioate; [N,N-bis(2-methoxyethyl)carbamoyl]ethyl methyl(2E)but-2-ene-1,4-dioate; (N,N-dimethylcarbamoyl)ethyl methyl(2E)but-2-ene-1,4-dioate; 2-{2-[(2E)-3-(methoxy carbonyl)prop-2-enoyloxyl]-N-methylacetylamin}acetic acid; (N-{[(tert-butyl)oxycarbonyl]methyl}carbamoyl)methyl methyl(2E)but-2-ene-1,4-dioate; (2E)but-methyl-N-{[(methylethyl)oxycarbonyl]methyl}carbamoyl)methyl(2E)but-2-ene-1,4-dioate; {N-[(ethoxycarbonyl)methyl]-N-benzylcarbamoyl}methyl methyl(2E)but-2-ene-1,4-dioate; {N-[(ethoxycarbonyl)methyl]-N-benzylcarbamoyl}ethyl methyl(2E)but-2-ene-1,4-dioate; {N-[(ethoxycarbonyl)methyl]-N-methylcarbamoyl}ethyl methyl(2E)but-2-ene-1,4-dioate; (1S)-1-methyl-2-morpholin-4-yl-2-oxo ethyl methyl(2E)but-2-ene-1,4-dioate; (1S)-1-[N,N-bis(2-methoxyethyl)carbamoyl]ethyl methyl(2E)but-2-ene-1,4-dioate; (1R)-1-(N,N-diethylcarbamoyl)ethyl methyl(2E)but-2-ene-1,4-dioate; and a pharmaceutically acceptable salt of any of the foregoing.

In certain embodiments of a compound of Formula (I), the compound is chosen from: (N,N-diethylcarbamoyl)methyl methyl(2E)but-2-ene-1,4-dioate; methyl[N-benzylcarbamoyl]methyl(2E)but-2-ene-1,4-dioate; methyl 2-morpholin-4-yl-2-oxoethyl(2E)but-2-ene-1,4-dioate; (N-butylcarbamoyl)methyl methyl(2E)but-2-ene-1,4-dioate; [N-(2-methoxyethyl)carbamoyl]methyl methyl(2E)but-2-ene-1,4-dioate; 2-{2-[(2E)-3-(methoxycarbonyl)prop-2-enoyloxy]acetylamino}acetic acid; {2-[(2E)-3-(methoxycarbonyl)prop-2-enoyloxy]acetylamino}butanoic acid; methyl(N-(1,3,4-thiadiazol-2-yl)carbamoyl)methyl(2E)but-2ene-1,4-dioate; (N,N-dimethylcarbamoyl)methyl methyl(2E)but-2-ene-1,4-dioate; (N-methoxy-N-methylcarbamoyl)methyl methyl(2E)but-2-ene-1,4-dioate; bis-(2-methoxyethylamino)carbamoyl]methyl methyl(2E)but-2-ene-1,4-dioate; [N-(methoxycarbonyl)carbamoyl]methyl methyl(2E)but-2ene-1,4-dioate; methyl 2-oxo-2-piperazinylethyl(2E)but-2-ene-1,4-dioate; methyl 2-oxo-2-(2-oxo(1,3-oxazolidin-3-yl)ethyl(2E)but-2ene-1,4-dioate; {N-[2-(dimethylamino)ethyl]carbamoyl}methyl methyl(2E)but-2ene-1,4-dioate; (N-[(methoxycarbonyl)ethyl]carbamoyl)methyl methyl(2E)but-2-ene-1,4-dioate; 2-{2-[(2E)-3-(methoxycarbonyl)prop-2-enoyloxy]acetylamino}propanoic acid; and a pharmaceutically acceptable salt of any of the foregoing.

In certain embodiments of a compound of Formula (I), R^{3a} and R^{4a} are independently chosen from hydrogen, C₁₋₆ alkyl, substituted C₁₋₆ alkyl, C₆₋₁₀ aryl, substituted C₆₋₁₀ aryl, C₄₋₁₂ cycloalkylalkyl, substituted C₄₋₁₂ cycloalkylalkyl, C₇₋₁₂ arylalkyl, substituted C₇₋₁₂ arylalkyl, C₁₋₆ heteroalkyl, substituted C₁₋₆ heteroalkyl, C₆₋₁₀ heteroaryl, substituted C₆₋₁₀ heteroaryl, C₄₋₁₂ heterocycloalkylalkyl, substituted C₄₋₁₂ heterocycloalkylalkyl, C₇₋₁₂ heteroarylalkyl, substituted C₇₋₁₂ heteroarylalkyl; or R^{3a} and R^{4a} together with the nitrogen to which they are bonded form a ring chosen from a C₅₋₁₀ heteroaryl, substituted C₅₋₁₀ heteroaryl, C₅₋₁₀ heterocycloalkyl, and substituted C₅₋₁₀ heterocycloalkyl.

In some embodiments, the compound that metabolizes to MMF is a compound of Formula II:

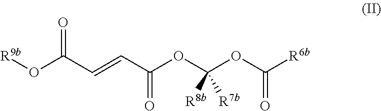

or a pharmaceutically acceptable salt thereof, wherein
R^{6b} is chosen from C₁₋₆ alkyl, substituted C₁₋₆ alkyl, C₁₋₆ heteroalkyl, substituted C₁₋₆ heteroalkyl, C₃₋₈ cycloalkyl, substituted C₃₋₈ cycloalkyl, C₆₋₈ aryl, substituted C₆₋₈ aryl, and —OR^{10b} wherein R^{10b} is chosen from C₁₋₆ alkyl, substituted C₁₋₆ alkyl, C₃₋₁₀ cycloalkyl, substituted C₃₋₁₀ cycloalkyl, C₆₋₁₀ aryl, and substituted C₆₋₁₀ aryl;
R^{7b} and R^{8b} are independently chosen from hydrogen, C₁₋₆ alkyl, and substituted C₁₋₆ alkyl; and
R^{9b} is chosen from C₁₋₆ alkyl and substituted C₁₋₆ alkyl;
wherein each substituent group is independently chosen from halogen, —OH, —CN, —CF₃, ═O, —NO₂, benzyl, —C(O)NR^{11b}₂, —R^{11b}, —OR^{11b}, —C(O)R^{11b}, —COOR^{11b}, and —NR^{11b}₂ wherein each R^{11b} is independently chosen from hydrogen and C₁₋₄ alkyl.

In certain embodiments of a compound of Formula (II), each substituent group is independently chosen from halogen, —OH, —CN, —CF₃, —R^{11b}, —OR^{11b}, and —NR^{11b}₂ wherein each R^{11b} is independently chosen from hydrogen and C₁₋₄ alkyl.

In certain embodiments of a compound of Formula (I), each substituent group is independently chosen from ═O, C₁₋₄ alkyl, and —COOR^{11b} wherein R^{11b} is chosen from hydrogen and C₁₋₄ alkyl.

In certain embodiments of a compound of Formula (II), one of R^{7b} and R^{8b} is hydrogen and the other of R^{7b} and R^{8b} is C₁₋₆ alkyl. In certain embodiments of a compound of Formula (II), one of R^{7b} and R^{8b} is hydrogen and the other of R^{7b} and R^{8b} is C₁₋₄ alkyl.

In certain embodiments of a compound of Formula (II), one of R^{7b} and R^{8b} is hydrogen and the other of R^{7b} and R^{8b} is chosen from methyl, ethyl, n-propyl, and isopropyl. In certain embodiments of a compound of Formula (II), each of R^{7b} and R^{8b} is hydrogen.

In certain embodiments of a compound of Formula (II), R^{9b} is chosen from substituted C₁₋₆ alkyl and —OR^{11b} wherein R^{11b} is independently C₁₋₄ alkyl.

In certain embodiments of a compound of Formula (II), R^{9b} is C₁₋₆ alkyl, in certain embodiments, R^{9b} is C₁₋₃ alkyl; and in certain embodiments, R^{9b} is chosen from methyl and ethyl.

In certain embodiments of a compound of Formula (II), R^{9b} is methyl.

In certain embodiments of a compound of Formula (II), R^{9b} is chosen from ethyl, n-propyl, isopropyl, n-butyl, sec-butyl, isobutyl, and tert-butyl.

In certain embodiments of a compound of Formula (II), R^{9b} is chosen from methyl, ethyl, n-propyl, isopropyl, n-butyl, isobutyl, and tert-butyl.

In certain embodiments of a compound of Formula (II), R^{6b} is C₁₋₆ alkyl; one of R^{7b} and R^{8b} is hydrogen and the other of R^{7b} and R^{8b} is C₁₋₆ alkyl; and R^{9b} is chosen from C₁₋₆ alkyl and substituted C₁₋₆ alkyl.

In certain embodiments of a compound of Formula (II), R^{6b} is —OR^{10b},

In certain embodiments of a compound of Formula (II), R^{10b} is chosen from C₁₋₄ alkyl, cyclohexyl, and phenyl.

In certain embodiments of a compound of Formula (II), R^{6b} is chosen from methyl, ethyl, n-propyl, and isopropyl; one of R^{7b} and R^{8b} is hydrogen and the other of R^{7b} and R^{8b} is chosen from methyl, ethyl, n-propyl, and isopropyl.

In certain embodiments of a compound of Formula (II), R^{6b} is substituted C₁₋₂ alkyl, wherein each of the one or more substituent groups are chosen from —COOH,

—NHC(O)CH₂NH₂, and —NH₂.

In certain embodiments of a compound of Formula (II), R^{6b} is chosen from ethoxy, methylethoxy, isopropyl, phenyl, cyclohexyl, cyclohexyloxy,

—CH(NH₂CH₂COOH, —CH₂CH(NH₂)COOH, —CH(NHC(O)CH₂NH₂)—CH₂COOH, and —CH₂CH(NHC(O)CH₂NH₂)—COOH.

In certain embodiments of a compound of Formula (II), R^{9b} is chosen from methyl and ethyl; one of R^{7b} and R^{8b} is hydrogen and the other of R^{7b} and R^{8b} is chosen from hydrogen, methyl, ethyl, n-propyl, and isopropyl; and R^{6b} is chosen from C₁₋₃ alkyl, substituted C₁₋₂ alkyl wherein each of the one or more substituent groups are chosen —COOH, —NHC(O)CH₂NH₂, and —NH₂, —OR^{10b} wherein R^{10b} is chosen from C₁₋₃ alkyl and cyclohexyl, phenyl, and cyclohexyl.

In certain embodiments of a compound of Formula (II), the compound is chosen from: ethoxycarbonyloxyethyl methyl(2E)but-2-ene-1,4-dioate; methyl(methylethoxycarbonyloxy)ethyl(2E)but-2-ene-1,4-dioate; (cyclohexyloxycarbonyloxy)ethyl methyl(2E)but-2-ene-1,4-dioate; and a pharmaceutically acceptable salt of any of the foregoing.

In certain embodiments of a compound of Formula (II), the compound is chosen from: methyl(2-methylpropanoyloxy)ethyl(2E)but-2-ene-1,4-dioate; methyl phenylcarbonyloxyethyl(2E)but-2-ene-1,4-dioate; cyclohexylcarbonyloxybutyl methyl(2E)but-2-ene-1,4-dioate; [(2E)-3-(methoxycarbonyl)prop-2-enoyloxy]ethyl methyl(2E)but-2-ene-1,4-dioate; methyl 2-methyl-1-phenylcarbonyloxypropyl(2E)but-2-ene-1,4-dioate; and a pharmaceutically acceptable salt of any of the foregoing.

In certain embodiments of a compound of Formula (II), the compound is chosen from: ethoxycarbonyloxyethyl methyl(2E)but-2-ene-1,4-dioate; methyl(methylethoxycarbonyloxy)ethyl(2E)but-2-ene-1,4-dioate; methyl(2-methylpropanoyloxy)ethyl(2E)but-2-ene-1,4-dioate; methyl phenylcarbonyloxyethyl(2E)but-2-ene-1,4-dioate; cyclohexylcarbonyloxybutyl methyl(2E)but-2-ene-1,4-dioate; [(2E)-3-(methoxycarbonyl)prop-2-enoyloxy]ethyl methyl(2E)but-2-ene-1,4-dioate; (cyclohexyloxycarbonyloxy)ethyl methyl(2E)but-2-ene-1,4-dioate; methyl 2-methyl-1-phenylcarbonyloxypropyl(2E)but-2-ene-1,4-dioate; 3-({[(2E)-3-(methoxycarbonyl)prop-2-enoyloxy]methyl}oxycarbonyl)(3S)-3-aminopropanoic acid, 2,2,2-trifluoroacetic acid; 3-({[(2E)-3-(methoxycarbonyl)prop-2-enoyloxy]methyl}oxycarbonyl)(2S)-2-aminopropanoic acid, 2,2,2-trifluoroacetic acid; 3-({[(2E)-3-(methoxycarbonyl)prop-2-enoyloxy]methyl}oxycarbonyl)(3S)-3-(2-aminoacetylamino)propanoic acid, 2,2,2-trifluoroacetic acid; 3-({[(2E)-3-(methoxycarbonyl)prop-2-enoyloxy]methyl}oxycarbonyl)(2S)-2-aminopropanoic acid, 2,2,2-trifluoroacetic acid; 3-{[(2E)-3-(methoxycarbonyl)prop-2enoyloxy]ethoxycarbonyloxy}(2S)-2-aminopropanoic acid, chloride; and a pharmaceutically acceptable salt of any of the foregoing.

The compounds of Formulae (I)-(II) may be prepared using methods known to those skilled in the art, or the methods disclosed in U.S. Pat. No. 8,148,414 B2.

In another embodiment is provided silicon-containing compounds, which like DMF and the compounds of Formulae (I)-(II), can metabolize into MMF upon administration.

In some embodiments, the compound that metabolizes to MMF is a compound of Formula (III):

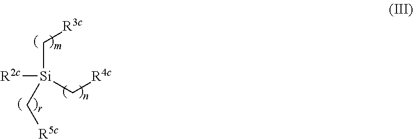

or a pharmaceutically acceptable salt thereof, wherein:
R^{2c} is C₁-C₁₀ alkyl, C₅-C₁₅ aryl, hydroxyl, —O—C₁-C₁₀ alkyl, or —O—C₅-C₁₅ aryl; each of R^{3c}, R^{4c}, and R^{5c}, independently, is C₁-C₁₀ alkyl, C₅-C₁₅ aryl, hydroxyl, —O—C₁-C₁₀ alkyl, —O—C₅-C₁₅ aryl, or

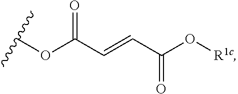

wherein R^{1c} is C₁-C₂₄ alkyl or C₅-C₅₀ aryl; each of which can be optionally substituted; and
each of m, n, and r, independently, is 0-4;
provided that at least one of R^{3c}, R^{4c}, and R^{5c} is

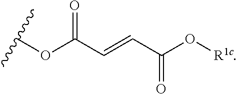

Another group of compounds of Formula III include compounds wherein R^{1c} is optionally substituted C₁-C₂₄ alkyl. Another group of compounds of Formula III include compounds wherein R^{1c} is optionally substituted C₁-C₆ alkyl. Another group of compounds of Formula III include compounds wherein R^{1c} is optionally substituted methyl, ethyl, or isopropyl. Another group of compounds of Formula III include compounds wherein R^{1c} is optionally substituted C₅-C₅₀ aryl. Another group of compounds of Formula III include compounds wherein R^{1c} is optionally substituted C₅-C₁₀ aryl. Another group of compounds of Formula III include compounds wherein R^{2c} is C₁-C₁₀ alkyl. Another group of compounds of Formula III include compounds wherein R^{2c} is optionally substituted C₁-C₆ alkyl. Another group of compounds of Formula III include compounds wherein R^{2c} is optionally substituted methyl, ethyl, or isopropyl. Another group of compounds of Formula III include compounds wherein R^{2c} is optionally substituted C₅-C₁₅ aryl. Another group of compounds of Formula III include compounds wherein R^{2c} is optionally substituted C₅-C₁₀ aryl.

In a further embodiment, the compound that metabolizes to MMF is a compound of Formula (III):

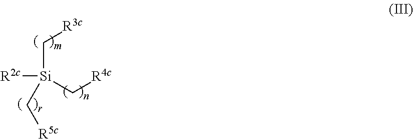

or a pharmaceutically acceptable salt thereof, wherein
R^{2c} is C₁-C₁₀ alkyl, C₆-C₁₀ aryl, hydroxyl, —O—C₁-C₁₀ alkyl, or —O—C₆-C₁₀ aryl;
each of R^{3c}, R^{4c}, and R^{5c}, independently, is C₁-C₁₀ alkyl, C₆-C₁₀ aryl, hydroxyl, —O—C₁-C₁₀ alkyl, —O—C₆-C₁₀ aryl, or

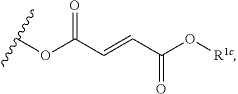

wherein R^{1c} is C₁-C₂₄ alkyl or C₆-C₁₀ aryl; each of which can be optionally substituted; and
each of m, n, and r, independently, is 0-4;
provided that at least one of R^{3c}, R^{4c}, and R^{5c} is

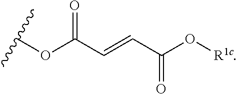

In some embodiments, the compound that metabolizes to MMF is chosen from (dimethylsilanediyl)dimethyl difumarate; methyl ((trimethoxysilyl)methyl)fumarate; methyl ((trihydroxysilyl)methyl)fumarate; trimethyl (methylsilanetriyl)trifumarate; and a pharmaceutically acceptable salt of any of the foregoing.

In some embodiments, the compound that metabolizes to MMF is a compound of Formula (IV):

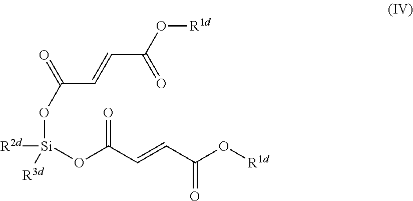

or a pharmaceutically acceptable salt thereof, wherein:
each R^{1d} is independently optionally substituted C₁-C₂₄ alkyl or C₅-C₅₀ aryl;
each of, independently, R^{2d} and R^{3d}, is C₁-C₁₀ alkyl or C₅-C₁₅ aryl.
R^{2d} and R^{3d} can be the same or different, can be optionally substituted, and independently can be selected from the group consisting of C₁-C₁₀ alkyl or C₅-C₁₅ aryl.

In another embodiment, compounds of Formula IV include compounds wherein each R^{1d} is independently optionally substituted C₁-C₂₄ alkyl or C₆-C₁₀ aryl. In another embodiment, compounds of Formula IV include compounds wherein R^{1d} is optionally substituted C₁-C₂₄ alkyl. Another group of compounds of Formula IV include compounds wherein R^{1d} is optionally substituted C₁-C₆ alkyl. Another group of compounds of Formula IV include compounds wherein R^{1d} is optionally substituted methyl, ethyl, or isopropyl. Another group of compounds of Formula IV include compounds wherein R^{1d} is optionally substituted C₅-C₅₀ aryl. Another group of compounds of Formula IV include compounds wherein R^{1d} is optionally substituted C₅-C₁₀ aryl. Another group of compounds of Formula IV include compounds wherein each of R^{2d} and R^{3d} is, independently, optionally substituted C₁-C₁₀ alkyl. Another group of compounds of Formula IV include compounds wherein each of R^{2d} and R^{3d} is, independently, optionally substituted C₁-C₆ alkyl. Another group of compounds of Formula IV include compounds wherein each of R^{2d} and R^{3d} is, independently, optionally substituted methyl, ethyl, or isopropyl. Another group of compounds of Formula IV include compounds wherein each of R^{2d} and R^{3d} is, independently, optionally substituted C₅-C₁₅ aryl. Another group of compounds of Formula IV include compounds wherein each of R^{2d} and R^{3d} is, independently, optionally substituted C₅-C₁₀ aryl.

In a further embodiment, the compound that metabolizes to MMF is a compound of Formula (IV):

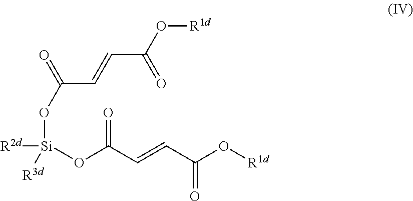

or a pharmaceutically acceptable salt thereof, wherein:
R^{1d} is C₁-C₂₄ alkyl or C₆-C₁₀ aryl; and
each of, independently, R^{2d} and R^{3d}, is C₁-C₁₀ alkyl or C₆-C₁₀ aryl.

In some embodiments, the compound that metabolizes to MMF is a compound of Formula (V):

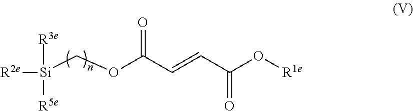

or a pharmaceutically acceptable salt thereof, wherein:
R^{1e} is C₁-C₂₄ alkyl or C₅-C₅₀ aryl;
each of R^{2e}, R^{3e}, and R^{5e}, independently, is hydroxyl, C₁-C₁₀ alkyl, C₅-C₁₅ aryl, —O—C₁-C₁₀ alkyl, or —O—C₅-C₁₅ aryl; and
   n is 1 or 2.

In another embodiment, compounds of Formula V include compounds wherein R^{1e} is optionally substituted C₁-C₂₄ alkyl. Another group of compounds of Formula V include compounds wherein R^{1e} is optionally substituted C₁-C₆ alkyl. Another group of compounds of Formula V include compounds wherein R^{1e} is optionally substituted methyl, ethyl, or isopropyl. Another group of compounds of Formula V include compounds wherein R^{1e} is optionally substituted C₅-C₅₀ aryl. Another group of compounds of Formula V include compounds wherein R^{1e} is optionally substituted C₅-C₁₀ aryl. Another group of compounds of Formula V include compounds wherein each of R^{2e}, R^{3e}, and R^{5e} is, independently, hydroxyl. Another group of compounds of Formula V include compounds wherein each of R^{2e}, R^{3e}, and R^{5e} is, independently, optionally substituted C₁-C₁₀ alkyl. Another group of compounds of Formula V include compounds wherein each of R^{2e}, R^{3e}, and R^{5e} is, independently, optionally substituted C₁-C₆ alkyl. Another group of compounds of Formula V include compounds wherein each of R^{2e}, R^{3e}, and R^{5e} is, independently, optionally substituted methyl, ethyl, or isopropyl. Another group of compounds of Formula V include compounds wherein each of R^{2e}, R^{3e}, and R₅ₑ is, independently, optionally substituted C₅-C₁₅ aryl. Another group of compounds of Formula V include compounds wherein each of R^{2e}, R^{3e}, and R^{5e} is, independently, optionally substituted C₅-C₁₀ aryl.

In a further embodiment, the compound that metabolizes to MMF is a compound of Formula (V):

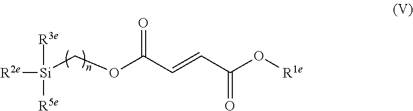

or a pharmaceutically acceptable salt thereof, wherein:
R^{1e} is C₁-C₂₄ alkyl or C₆-C₁₀ aryl;
each of R^{2e}, R^{3e}, and R^{5e}, independently, is hydroxyl, C₁-C₁₀ alkyl, C₆-C₁₀ aryl, —O—C₁-C₁₀ alkyl, or —O—C₆-C₁₀ aryl; and
   n is 1 or 2.

In some embodiments, the compound that metabolizes to MMF is a compound of Formula (VI):

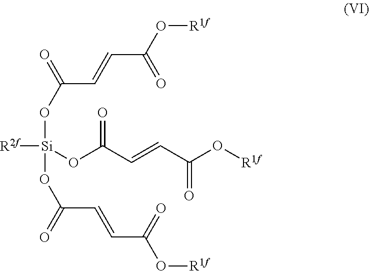

or a pharmaceutically acceptable salt thereof, wherein:
R^{1f} is C₁-C₂₄ alkyl or C₅-C₅₀ aryl; and

R^{2f} is C₁-C₁₀ alkyl.

In another embodiment, compounds of Formula VI include compounds wherein R^{1f} is optionally substituted C₁-C₂₄ alkyl. Another group of compounds of Formula VI include compounds wherein R^{1f} is optionally substituted C₁-C₆ alkyl. Another group of compounds of Formula VI include compounds wherein R^{1f} is optionally substituted methyl, ethyl, or isopropyl. Another group of compounds of Formula VI include compounds wherein R^{1f} is optionally substituted C₅-C₅₀ aryl. Another group of compounds of Formula VI include compounds wherein R^{1f} is optionally substituted C₅-C₁₀ aryl. Another group of compounds of Formula VI include compounds wherein R^{2f} is optionally substituted C₁-C₆ alkyl. Another group of compounds of Formula VI include compounds wherein R^{2f} is optionally substituted methyl, ethyl, or isopropyl.

In a further embodiment, the compound that metabolizes to MMF is a compound of Formula (VI):

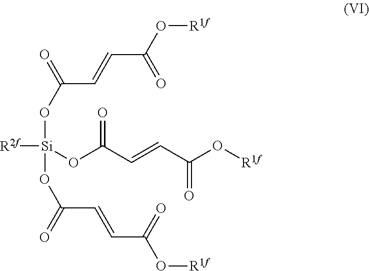

or a pharmaceutically acceptable salt thereof, wherein:

R^{1f} is C₁-C₂₄ alkyl or C₆-C₁₀ aryl; and

R^{2f} is C₁-C₁₀ alkyl.

In another aspect, the invention features, a method of treating a subject having a natural killer (NK) function related disorder or condition comprising: administering to the subject in need of treatment a dialkyl fumarate in an amount sufficient to treat the disorder,

wherein the disorder or condition is selected from:

cancer, a viral infection, and inflammation.

In an embodiment, the dialkyl fumarate is:

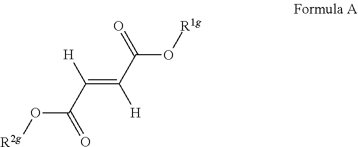

wherein R^{1g} and R^{2g}, which may be the same or different, independently represent a linear, branched or cyclic, saturated or unsaturated C₁₋₂₀ alkyl radical which may be optionally substituted with halogen (Cl, F, I, Br), hydroxy, C₁₋₄ alkoxy, nitro or cyano.

In an embodiment, R^{1g} and R^{2g}, which may be the same or different, independently are methyl, ethyl, n-propyl, isopropyl, n-butyl, sec-butyl, t-butyl, pentyl, cyclopentyl, 2-ethyl hexyl, hexyl, cyclohexyl, heptyl, cycloheptyl, octyl, vinyl, allyl, 2-hydroxy ethyl, 2 or 3-hydroxy propyl, 2-methoxy ethyl, methoxy methyl or 2- or 3-methoxy propyl.

In an embodiment, R^{1g} and R^{2g} are identical and are methyl or ethyl.

In an embodiment, R^{1g} and R^{2g} are methyl.

In an embodiment, the compound is a monoalkyl fumarate. In an embodiment, the monoalkyl fumarate is:

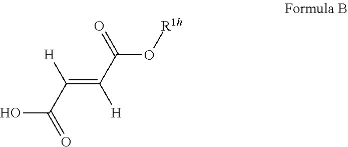

wherein R^{1h} represents a linear, branched or cyclic, saturated or unsaturated C₁₋₂₀ alkyl radical which may be optionally substituted with halogen (Cl, F, I, Br), hydroxy, C₁₋₄ alkoxy, nitro or cyano;
or a pharmaceutically acceptable salt thereof.

In an embodiment, R^{1h} is methyl, ethyl, n-propyl, isopropyl, n-butyl, sec-butyl, t-butyl, pentyl, cyclopentyl, 2-ethyl hexyl, hexyl, cyclohexyl, heptyl, cycloheptyl, octyl, vinyl, allyl, 2-hydroxy ethyl, 2 or 3-hydroxy propyl, 2-methoxy ethyl, methoxy methyl or 2- or 3-methoxy propyl.

In an embodiment, R^{1h} is methyl or ethyl.

In an embodiment, R^{1h} is methyl.

In a further embodiment, the compound that metabolizes to MMF is a compound of Formula (VII):

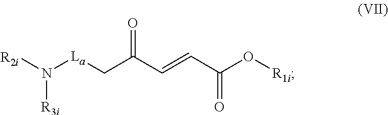

wherein:
R₁ᵢ is unsubstituted C₁-C₆ alkyl;
Lₐ is substituted or unsubstituted C₁-C₆ alkyl linker, substituted or unsubstituted C₃-C₁₀ carbocycle, substituted or unsubstituted C₆-C₁₀ aryl, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S; and
R₂ᵢ and R₃ᵢ are each, independently, H, substituted or unsubstituted C₁-C₆ alkyl, substituted or unsubstituted C₂-C₆ alkenyl, substituted or unsubstituted C₂-C₆ alkynyl, substituted or unsubstituted C₆-C₁₀ aryl, substituted or unsubstituted C₃-C₁₀ carbocycle, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S;
or alternatively, R₂ᵢ and R₃ᵢ, together with the nitrogen atom to which they are attached, form a substituted or unsubstituted heteroaryl comprising one or two 5- or 6-member rings and 1-4 heteroatoms selected from N, O and S or a substituted or unsubstituted heterocycle comprising one or two 5- or 6-member rings and 1-4 heteroatoms selected from N, O and S.
In some embodiments, the compound of Formula (VII) is selected from a compound of Formula (VIIa):

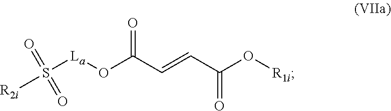

wherein:
R₁ᵢ is unsubstituted C₁-C₆ alkyl;
Lₐ is substituted or unsubstituted C₁-C₆ alkyl linker, substituted or unsubstituted C₃-C₁₀ carbocycle, substituted or unsubstituted C₆-C₁₀ aryl, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S; and
R₂ᵢ is H, substituted or unsubstituted C₁-C₆ alkyl, substituted or unsubstituted C₂-C₆ alkenyl, substituted or unsubstituted C₂-C₆ alkynyl, substituted or unsubstituted C₆-C₁₀ aryl, substituted or unsubstituted C₃-C₁₀ carbocycle, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S.

In some embodiments, the compound of Formula (VII) is selected from a compound of Formula (VIIb):

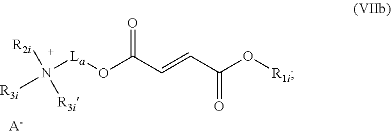

A⁻ is a pharmaceutically acceptable anion;
R₁ᵢ is unsubstituted C₁-C₆ alkyl;
Lₐ is substituted or unsubstituted C₁-C₆ alkyl linker, substituted or unsubstituted C₃-C₁₀ carbocycle, substituted or unsubstituted C₆-C₁₀ aryl, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S;
R₃ᵢ′ is substituted or unsubstituted C₁-C₆ alkyl; and
R₂ᵢ and R₃ᵢ are each, independently, H, substituted or unsubstituted C₁-C₆ alkyl, substituted or unsubstituted C₂-C₆ alkenyl, substituted or unsubstituted C₂-C₆ alkynyl, substituted or unsubstituted C₆-C₁₀ aryl, substituted or unsubstituted C₃-C₁₀ carbocycle, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S;
or alternatively, R₂ᵢ and R₃ᵢ, together with the nitrogen atom to which they are attached, form a substituted or unsubstituted heteroaryl comprising one or two 5- or 6-member rings and 1-4 heteroatoms selected from N, O and S, or a substituted or unsubstituted heterocycle comprising one or two 5- or 6-member rings and 1-4 heteroatoms selected from N, O and S.

In some embodiments, the compound of Formula (VII) is selected from a compound of Formula (VIII):

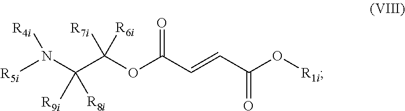

wherein:
R₁ᵢ is unsubstituted C₁-C₆ alkyl;
R₄ᵢ and R₅ᵢ are each, independently, H, substituted or unsubstituted C₁-C₆ alkyl, substituted or unsubstituted C₂-C₆ alkenyl, substituted or unsubstituted C₂-C₆ alkynyl, substituted or unsubstituted C₆-C₁₀ aryl, substituted or unsubstituted C₃-C₁₀ carbocycle, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S;
R₆ᵢ, R₇ᵢ, R₈ᵢ and R₉ᵢ are each, independently, H, substituted or unsubstituted C₁-C₆ alkyl, substituted or unsubstituted C₂-C₆ alkenyl, substituted or unsubstituted C₂-C₆ alkynyl or C(O)ORₐ; and
Rₐ is H or substituted or unsubstituted C₁-C₆ alkyl.

In some embodiments, the compound of Formula (VII) is selected from a compound of Formula (IX):

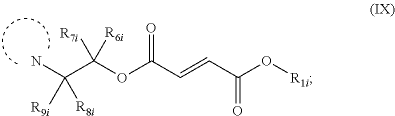

wherein:
R₁ᵢ is unsubstituted C₁-C₆ alkyl;

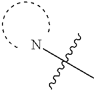 [SMILES: *N]

is selected from the group consisting of:

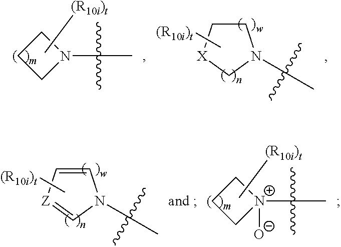

### X is N, O, S or SO₂;

### Z is C or N;

m is 0, 1, 2, or 3;
n is 1 or 2;
w is 0, 1, 2 or 3;
t is 0, 1, 2, 3, 4, 5, 6, 7, 8, 9 or 10;
R₆ᵢ, R₇ᵢ, R₈ᵢ and R₉ᵢ are each, independently, H, substituted or unsubstituted C₁-C₆ alkyl, substituted or unsubstituted C₂-C₆ alkenyl, substituted or unsubstituted C₂-C₆ alkynyl or C(O)ORₐ; and
Rₐ is H or substituted or unsubstituted C₁-C₆ alkyl; and
each R₁₀ᵢ is, independently, H, halogen, substituted or unsubstituted C₁-C₆ alkyl, substituted or unsubstituted C₂-C₆ alkenyl, substituted or unsubstituted C₂-C₆ alkynyl, substituted or unsubstituted C₃-C₁₀ carbocycle, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S;
or, alternatively, two R₁₀ᵢ's attached to the same carbon atom, together with the carbon atom to which they are attached, form a carbonyl, substituted or unsubstituted C₃-C₁₀ carbocycle, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S;
or, alternatively, two R₁₀ᵢ's attached to different atoms, together with the atoms to which they are attached, form a substituted or unsubstituted C₃-C₁₀ carbocycle, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S.

In some embodiments, the compound is a compound listed in Table A herein.

Representative compounds of the present invention include compounds listed in Table A.

TABLE A

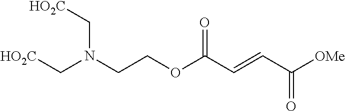 [SMILES: COC(=O)/C=C/C(=O)OCCN(CC(=O)O)CC(=O)O]

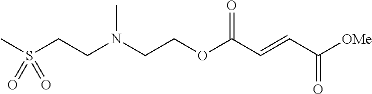 [SMILES: COC(=O)/C=C/C(=O)OCCN(C)CCS(C)(=O)=O]

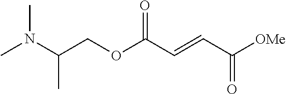

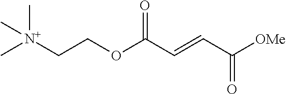

A⁻

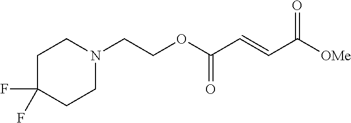 [SMILES: COC(=O)/C=C/C(=O)OCCN1CCC(F)(F)CC1]

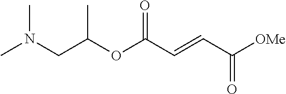

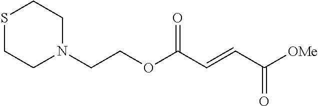 [SMILES: COC(=O)/C=C/C(=O)OCCN1CCSCC1]

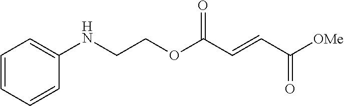 [SMILES: COC(=O)/C=C/C(=O)OCCNc1ccccc1]

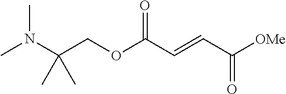 [SMILES: COC(=O)/C=C/C(=O)OCC(C)(C)N(C)C]

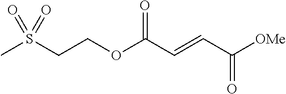

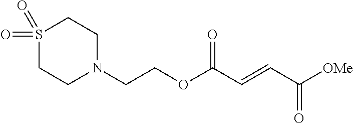 [SMILES: COC(=O)/C=C/C(=O)OCCN1CCS(=O)(=O)CC1]

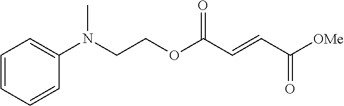 [SMILES: COC(=O)/C=C/C(=O)OCCN(C)c1ccccc1]

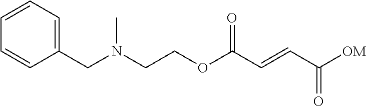

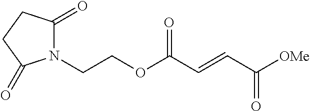 [SMILES: COC(=O)/C=C/C(=O)OCCN1C(=O)CCC1=O]

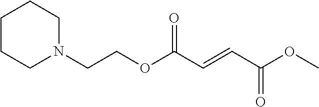 [SMILES: COC(=O)/C=C/C(=O)OCCN1CCCCC1]

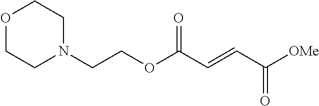 [SMILES: COC(=O)/C=C/C(=O)OCCN1CCOCC1]

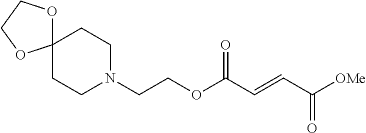 [SMILES: COC(=O)/C=C/C(=O)OCCN1CCC2(CC1)OCCO2]

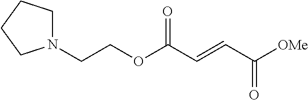 [SMILES: COC(=O)/C=C/C(=O)OCCN1CCCC1]

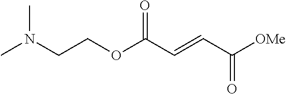

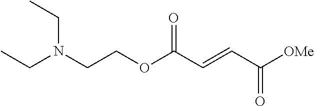 [SMILES: CCN(CC)CCOC(=O)/C=C/C(=O)OC]

A⁻ is a pharmaceutically acceptable anion.

In some embodiments, the compound of Formula (VII) is the compound (X):

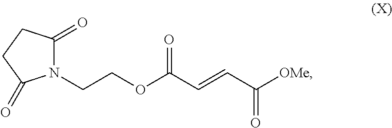 [SMILES: COC(=O)/C=C/C(=O)OCCN1C(=O)CCC1=O]

or a pharmaceutically acceptable salt thereof.

In an embodiment, the disorder or condition is cancer.

In an embodiment, the disorder or condition is a hematological malignancy.

In an embodiment, the hematological malignancy is selected from lymphocytic leukemia, chronic lymphocytic leukemia, and lymphoma.

In an embodiment, the disorder or condition is a solid tumor.

In an embodiment, the solid tumor is selected from gastrointestinal sarcoma, neuroblastoma, and kidney cancer.

In an embodiment, the disorder or condition is a viral infection.

In another aspect, the invention features, a method of treating a disorder or condition described herein by administering to the subject: a dialkyl fumarate, e.g., DMF, MMF, or a combination thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 depicts exemplary monomethyl fumarate (MMF) exposures after oral dimethyl fumarate (DMF) or MMF dosing. Satellite 5 animals per group were dosed orally with DMF or MMF (100 mg/kg) and sacrificed 30 minutes post-dosing. MMF exposures were determined and compared in various compartments. MMF levels were highly comparable between DMF and MMF dosing, suggesting any subsequent differences in pharmacodynamic responses would not simply be due to different exposures.

FIG. 2A depicts an exemplary overview of Venn diagrams comparing differentially expressed genes in each tissue. Data are presented as an aggregation of three time points for each tissue. FIG. 2B depicts exemplary Venn diagrams comparing differentially expressed genes in whole blood, cortex, hippocampus, striatum, jejunum, kidney, liver and spleen.

FIG. 3 depicts exemplary analysis of whole blood DMF differentially expressed genes (DEGs) in various pathways. Common effects were observed on NK cell function.

FIG. 4 depicts exemplary analysis of cortical MMF DEGs in various pathways.

FIG. 5 depicts exemplary analysis of hippocampal MMF DEGs in various pathways.

FIG. 6 depicts exemplary analysis of striatal MMF DEGs in various pathways.

FIG. 7 depicts exemplary analysis of jejunum DEGs in various pathways (A) DMF and MMF common DEGs; and (B) MMF DEGs.

FIG. 8 depicts exemplary analysis of kidney DEGs in various pathways (A) DMF and MMF common DEGs. DMF and MMF common DEGs showed good representation of Nrf2 pathway activation and xenobiotic metabolism, with particular emphasis on glutathione biosynthesis. (B) DMF specific DEGs; (C) MMF specific DEGs.

FIG. 9 depicts exemplary analysis of liver DMF and MMF common DEGs in various pathways.

FIG. 10 depicts an exemplary flow cytometry gating strategy for comparative analysis of DMF and MMF on Natural Killer (NK) cell phenotype. Protein expression is quantified by mean fluorescent intensity (MFI).

FIG. 11 depicts an exemplary comparative analysis of DMF and MMF on NK cell phenotype using markers: NK1.1 (klrb1b), Nkg2d (klrk1), NKp46 (ncr1), Nkg2a (klrc1), and CD94 (klrd1). Study design: Naïve C57Bl/6 mice were dosed PO with 100 mpk dimethyl fumarate (DMF) or molar equivalency of monomethyl fumarate (MMF). 12-hours post-dose, mice were sacrificed and blood, spleen, and inguinal lymph node were collected for analysis by flow cytometry.

FIG. 12 depicts an exemplary Venn diagram showing significant overlap of DEGs found in EAE and naïve brains with MMF treatment. The probability of this magnitude of overlap is 5.67×10⁻⁹⁴.

FIGS. 13A and 13B depict an exemplary Venn diagram showing number of DEGs identified in comparisons of DMF and MMF with vehicle. The numbers of DEGs from the DMF-vs-Vehicle and MMF-vs-Vehicle contrasts are depicted in the Venn diagrams for each tissue studied. A. Results from the single dosing regimen; DEGs from the 2 h, 7 h, and 12 h time points were pooled. B. Results from the multi-dosing regimen.

FIG. 14 depicts exemplary gene expression profiles from the spleen, which can differentiate DMF- and MMF-treated mice. Fifty-two genes in the spleens from multi-dosed naïve mice were found to be differentially expressed between DMF- and MMF-treated mice with a minimal fold change of 1.5-fold and p-value <0.001, adjusted for multiple comparisons. The normalized intensities of these 52 probe sets were clustered. Red columns represent MMF-treated mice, yellow represents Vehicle, and blue represents DMF-treated mice. Data were analyzed from 9 DMF-treated mice, 10 MMF-treated mice, and 9 Vehicle-treated mice.

FIG. 15 depicts exemplary analysis of DEPP up-regulation in the spinal cords and brains of DMF-treated animals. Naïve mice that were administered a multi-dosing regimen of DMF exhibited a significant increase in DEPP mRNA as represented by the Affymetrix probe sets 1433836_PM_a_at and 1433837_PM_at. **P<0.001, adjusted for multiple comparisons.

FIG. 16 is a schematic depicting that IL21 or NFkB may be activated in the spleens of DMF-treated mice. A subset of 4 genes from the 52 DEGs that differentiate DMF from MMF treatment in the spleen suggest that IL2 or NFkB may be activated. Pathway analysis was performed in and figures were derived from the Ingenuity IPA software.

FIG. 17 depicts an exemplary immunophenotyping panel for analysis of immune cell subsets in EAE mice treated with DMF or MMF.

FIG. 18 depicts exemplary NK cell analysis in blood and spleen and EAE clinical score analysis for a chronic dosing experiment in EAE mice.

FIG. 19 depicts exemplary NK cell protein expression in spleen and blood for a chronic dosing experiment in EAE mice.

FIG. 20 depicts exemplary NK cell subset and protein expression analysis in spleen and blood for a chronic dosing experiment in EAE mice.

FIG. 21 depicts exemplary NK cell analysis in spleen, iLN, and blood for a single dose experiment in EAE mice.

FIG. 22 depicts exemplary NK cell protein expression in spleen, iLN and blood for a single dose experiment in EAE mice.

FIG. 23 depicts exemplary NK cell subset and protein expression analysis in spleen, iLN and blood for a single dose experiment in EAE mice.

FIG. 24 depicts an exemplary flow cytometry gating strategy for comparative analysis of DMF and MMF on T cell phenotype. Protein expression is quantified by mean fluorescent intensity (MFI).

FIG. 25 depicts exemplary T regulatory cell analysis in spleen, iLN and blood for a chronic dosing experiment in EAE mice.

FIG. 26 depicts exemplary CD4+ cell subset and protein expression analysis in spleen, iLN and blood for a chronic dosing experiment in EAE mice.

FIG. 27 depicts exemplary CD8+ cell subset and protein expression analysis in spleen, iLN and blood for a chronic dosing experiment in EAE mice.

FIG. 28 depicts exemplary CD4+ T cell subset vs. EAE clinical score analysis in spleen for a chronic dosing experiment in EAE mice.

FIG. 29 depicts exemplary CD8+ T cell subset vs. EAE clinical score analysis in spleen for a chronic dosing experiment in EAE mice.

FIG. 30 depicts exemplary B cell analysis in naïve, vehicle, MMF or DMF treated EAE mice.

FIG. 31 depicts an exemplary myeloid cell gating strategy for comparative analysis of DMF and MMF on myeloid cell phenotype. (Swirski, F. K. et al. Identification of splenic reservoir monocytes and their deployment to inflammatory sites. Science 325, 612-616 (2009))

FIG. 32 depicts exemplary myeloid cell subset analysis in spleen and iLN for a chronic dosing experiment in EAE mice.

FIG. 33 depicts exemplary cumulative EAE scores manifest as differences in global gene expression patterns Genes from the spinal cord of chronically-dosed 7 h EAE mice that exhibited an average normalized intensity greater than 4 and coefficient of variation (CV) greater than 0.05 were selected (2,872 total genes). The normalized intensities of these genes were subjected to unsupervised clustering. The purple bar on top of the figure is indicative of the cumulative EAE score of each animal; the darker the shade, the higher the disease score.

FIG. 34 depicts an exemplary Venn diagram showing number of DEGs identified in comparisons of DMF and MMF with Vehicle.

FIG. 35 depicts exemplary gene expression profiles from the brain that can differentiate DMF- and MMF-treated EAE mice. A set of 31 genes was found to be differentially expressed in the brains of mice chronically-dosed with DMF and MMF. The normalized intensities of these genes were subjected to unsupervised clustering.

FIGS. 36A and 36B depict exemplary analysis of Zbtb16 transcript increase in DMF-treated animals. Boxplots of the normalized intensities for Affymetrix probe sets that represent the Zbtb16 transcript are shown for chronically-dosed animals, 12 h after the final dosing. (A) Lymph Node, and (B) Spleen. *P<0.001, adjusted for multiple comparisons.

### DETAILED DESCRIPTION

The invention is based, at least in part, on the discovery that the prodrug DMF makes a contribution to pharmacologic effect that is distinct from that of its metabolite, MMF.

### DEFINITIONS

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a physical process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non covalent bond, between a first and a second atom of the analyte; or by changing the structure of a reagent, or a fragment or other derivative thereof, e.g., by breaking or forming a covalent or non covalent bond, between a first and a second atom of the reagent.

“Acquiring a sample” as the term is used herein, refers to obtaining possession of a sample, e.g., a tissue sample or nucleic acid sample, by “directly acquiring” or “indirectly acquiring” the sample. “Directly acquiring a sample” means performing a process (e.g., performing a physical method such as a surgery or extraction) to obtain the sample. “Indirectly acquiring a sample” refers to receiving the sample from another party or source (e.g., a third party laboratory that directly acquired the sample). Directly acquiring a sample includes performing a process that includes a physical change in a physical substance, e.g., a starting material, such as a tissue, e.g., a tissue in a human patient or a tissue that has was previously isolated from a patient. Exemplary changes include making a physical entity from a starting material, dissecting or scraping a tissue; separating or purifying a substance (e.g., a sample tissue or a nucleic acid sample); combining two or more separate entities into a mixture; performing a chemical reaction that includes breaking or forming a covalent or non-covalent bond. Directly acquiring a sample includes performing a process that includes a physical change in a sample or another substance, e.g., as described above.

A dimethyl fumarate (DMF)-differentially expressed gene, as the term is used herein, is a gene, the expression of which differs in a subject that has been administered DMF, as compared to a subject not administered DMF. Differential expression can be manifest at the transcriptional or translation level, e.g., at the level of mRNA or protein, or at both. By way of example, a gene is DMF-differentially expressed if the levels of the RNA or protein product, or both, of the gene are higher, in a subject administered DMF, as compared to a subject not administered DMF. A DMF-differentially expressed gene can be characterized by differential expression at one or both of the transcriptional and translational levels. In an embodiment a DMF-differentially expressed gene will not also be MMF-differentially expressed, or the differential expression for DMF will differ from the differential expression seen for MMF.

A prodrug (PD)-differentially expressed gene, as the term is used herein, is a gene, the expression of which differs in a subject that has been administered a prodrug, as compared to a subject not administered a prodrug. Differential expression can be manifest at the transcriptional or translation level, e.g., at the level of mRNA or protein, or at both. By way of example, a gene is PD-differentially expressed if the levels of the RNA or protein product, or both, of the gene are higher, in a subject administered prodrug, as compared to a subject not administered prodrug, e.g., DMF. A PD-differentially expressed gene can be characterized by differential expression at one or both of the transcriptional and translational levels. In an embodiment a PD-differentially expressed gene will not also be drug, e.g., MMF-differentially expressed, or the differential expression for PD will differ from the differential expression seen for drug, e.g., MMF.

A monomethyl fumarate (MMF)-differentially expressed gene, as the term is used herein, is a gene, the expression of which differs in a subject that has been administered MMF, as compared to a subject not administered MMF. Differential expression can be manifest at the transcriptional or translation level, e.g., at the level of mRNA or protein, or at both. By way of example, a gene is MMF-differentially expressed if the levels of the RNA or protein product, or both, of the gene are higher, in a subject administered MMF, as compared to a subject not administered MMF. An MMF-differentially expressed gene can be characterized by differential expression at one or both of the transcriptional and translational levels. In an embodiment an MMF-differentially expressed gene will not also be DMF-differentially expressed, or the differential expression for MMF will differ from the differential expression seen for DMF.

A DMF/MMF-differentially expressed gene, as the term is used herein, is a gene that is differentially expressed for both DMF and MMF.

A drug-differentially expressed gene, as the term is used herein, is a gene, the expression of which differs in a subject that has been administered drug, e.g., MMF, as compared to a subject not administered the drug. Differential expression can be manifest at the transcriptional or translation level, e.g., at the level of mRNA or protein, or at both. By way of example, a gene is drug-differentially expressed if the levels of the RNA or protein product, or both, of the gene are higher, in a subject administered drug, as compared to a subject not administered drug. A drug-differentially expressed gene can be characterized by differential expression at one or both of the transcriptional and translational levels. In an embodiment a drug-differentially expressed gene will not also be PD-differentially expressed, or the differential expression for drug will differ from the differential expression seen for prodrug.

A Drug/PD-differentially expressed gene, as the term is used herein, is a gene that is differentially expressed for both prodrug and drug.

As used herein, the “Expanded Disability Status Scale” or “EDSS” is intended to have its customary meaning in the medical practice. EDSS is a rating system that is frequently used for classifying and standardizing MS. The accepted scores range from 0 (normal) to 10 (death due to MS). Typically patients having an EDSS score of about 6 will have moderate disability (e.g., walk with a cane), whereas patients having an EDSS score of about 7 or 8 will have severe disability (e.g., will require a wheelchair). More specifically, EDSS scores in the range of 1-3 refer to an MS patient who is fully ambulatory, but has some signs in one or more functional systems; EDSS scores in the range higher than 3 to 4.5 show moderate to relatively severe disability; an EDSS score of 5 to 5.5 refers to a disability impairing or precluding full daily activities; EDSS scores of 6 to 6.5 refer to an MS patient requiring intermittent to constant, or unilateral to bilateral constant assistance (cane, crutch or brace) to walk; EDSS scores of 7 to 7.5 means that the MS patient is unable to walk beyond five meters even with aid, and is essentially restricted to a wheelchair; EDSS scores of 8 to 8.5 refer to patients that are restricted to bed; and EDSS scores of 9 to 10 mean that the MS patient is confined to bed, and progressively is unable to communicate effectively or eat and swallow, until death due to MS.

As used herein, the term “probe” refers to any molecule which is capable of selectively binding to a specifically intended target molecule, for example, a transcription product, e.g., an mRNA or cDNA, or a translation product, e.g., a polypeptide or protein. Probes can be either synthesized by one skilled in the art, or derived from appropriate biological preparations. For purposes of detection of the target molecule, probes can be specifically designed to be labeled, as described herein. Examples of molecules that can be utilized as probes include, but are not limited to, RNA, DNA, proteins, antibodies, and organic monomers.

As used herein, the term prodrug, or pro-drug, refers to a compound that is processed, in the body of a subject, into a drug. In an embodiment the processing comprises the breaking or formation of a bond, e.g., a covalent bond. Typically, breakage of a covalent bond releases the drug.

“Sample,” “tissue sample,” “subject or patient sample,” “subject or patient cell or tissue sample” or “specimen” each refers to a biological sample obtained from a tissue, e.g., a bodily fluid, of a subject or patient. The source of the tissue sample can be solid tissue as from a fresh, frozen and/or preserved organ, tissue sample, biopsy, or aspirate; blood or any blood constituents (e.g., serum, plasma); bodily fluids such as cerebral spinal fluid, whole blood, plasma and serum. The sample can include a non-cellular fraction (e.g., plasma, serum, or other non-cellular body fluid). In one embodiment, the sample is a serum sample. In other embodiments, the body fluid from which the sample is obtained from an individual comprises blood (e.g., whole blood). In certain embodiments, the blood can be further processed to obtain plasma or serum. In another embodiment, the sample contains a tissue, cells (e.g., peripheral blood mononuclear cells (PBMC)). In an embodiment the sample includes NK cells. For example, the sample can be a fine needle biopsy sample, an archival sample (e.g., an archived sample with a known diagnosis and/or treatment history), a histological section (e.g., a frozen or formalin-fixed section, e.g., after long term storage), among others. The term sample includes any material obtained and/or derived from a biological sample, including a polypeptide, and nucleic acid (e.g., genomic DNA, cDNA, RNA) purified or processed from the sample. Purification and/or processing of the sample can involve one or more of extraction, concentration, antibody isolation, sorting, concentration, fixation, addition of reagents and the like. The sample can contain compounds that are not naturally intermixed with the tissue in nature such as preservatives, anticoagulants, buffers, fixatives, nutrients, antibiotics or the like.

The term “alkyl” as employed herein by itself or as part of another group refers to both straight and branched chain radicals of up to 24 carbons. Alkyl groups include straight-chained and branched C₁-C₂₄ alkyl groups, e.g., C₁-C₁₀ alkyl groups. C₁-C₁₀ alkyl groups include methyl, ethyl, propyl, isopropyl, butyl, isobutyl, sec-butyl, tert-butyl, pentyl, isopentyl, neopentyl, hexyl, isohexyl, 3-methylpentyl, 2,2-dimethylbutyl, 2,3-dimethylbutyl, heptyl, 1-methylhexyl, 2-ethylhexyl, 1,4-dimethylpentyl, octyl, nonyl, and decyl. Unless otherwise indicated, all alkyl groups described herein include both unsubstituted and substituted alkyl groups. Further, each alkyl group can include its deuterated counterparts.

The term “heteroalkyl” is an alkyl group in which one to five carbons in the alkyl chain are replace by an independently selected oxygen, nitrogen or sulfur atom.

The term “aryl” as employed herein by itself or as part of another group refers to monocyclic, bicyclic, or tricyclic aromatic hydrocarbon containing from 5 to 50 carbons in the ring portion. Aryl groups include C₅₋₁₅ aryl, e.g., phenyl, p-tolyl, 4-methoxyphenyl, 4-(tert-butoxy)phenyl, 3-methyl-4-methoxyphenyl, 4-fluorophenyl, 4-chlorophenyl, 3-nitrophenyl, 3-aminophenyl, 3-acetamidophenyl, 4-acetamidophenyl, 2-methyl-3-acetamidophenyl, 2-methyl-3-aminophenyl, 3-methyl-4-aminophenyl, 2-amino-3-methylphenyl, 2,4-dimethyl-3-aminophenyl, 4-hydroxyphenyl, 3-methyl-4-hydroxyphenyl, 1-naphthyl, 3-amino-naphthyl, 2-methyl-3-amino-naphthyl, 6-amino-2-naphthyl, 4,6-dimethoxy-2-naphthyl, indanyl, biphenyl, phenanthryl, anthryl, and acenaphthyl. Unless otherwise indicated, all aryl groups described herein include both unsubstituted and substituted aryl groups.

The term “arylalkyl” refers to an alkyl group which is attached to another moiety through an alkyl group.

“Halogen” or “halo” may be fluoro, chloro, bromo or iodo.

The term “cycloalkyl” refers to completely saturated monocyclic, bicyclic or tricyclic hydrocarbon groups of 3-12 carbon atoms, preferably 3-9, or more preferably 3-8 carbon atoms. Exemplary monocyclic cycloalkyl groups include, but are not limited to, cyclopropyl, cyclobutyl, cyclopentyl, and cyclohexyl. Exemplary bicyclic cycloalkyl groups include bornyl, decahydronaphthyl, bicyclo[2.1.1]hexyl, bicyclo[2.2.1]heptyl, 6,6-dimethylbicyclo[3.1.1]heptyl, 2,6,6-trimethylbicyclo[3.1.1]heptyl, or bicyclo[2.2.2]octyl. Exemplary tricyclic carbocyclyl groups include adamantyl.

The term “cycloalkylalkyl” refers to a cycloalkyl group which is attached to another moiety through an alkyl group.

The term “heterocycloalkyl” refers to completely saturated monocyclic, bicyclic or tricyclic heterocyclyl comprising 3-15 ring members, at least one of which is a heteroatom, and up to 10 of which may be heteroatoms, wherein the heteroatoms are independently selected from O, S and N, and wherein N and S can be optionally oxidized to various oxidation states. Examples of heterocycloalkyl groups include [1,3]dioxolane, 1,4-dioxane, 1,4-dithiane, piperazinyl, 1,3-dioxolane, imidazolidinyl, imidazolinyl, pyrrolidine, dihydropyran, oxathiolane, dithiolane, I,3-dioxane, 1,3-dithianyl, oxathianyl, thiomorpholinyl, oxiranyl, aziridinyl, oxetanyl, azetidinyl, tetrahydrofuranyl, pyrrolidinyl, tetrahydropyranyl, piperidinyl, morpholinyl, and piperazinyl.

As used herein, the term “heteroaryl” refers to a 5-14 membered monocyclic-, bicyclic-, or tricyclic-ring system, having 1 to 10 heteroatoms independently selected from N, O or S, wherein N and S can be optionally oxidized to various oxidation states, and wherein at least one ring in the ring system is aromatic. In one embodiment, the heteroaryl is monocyclic and has 5 or 6 ring members. Examples of monocyclic heteroaryl groups include pyridyl, thienyl, furanyl, pyrrolyl, pyrazolyl, imidazoyl, oxazolyl, isoxazolyl, thiazolyl, isothiazolyl, triazolyl, oxadiazolyl, thiadiazolyl and tetrazolyl. In another embodiment, the heteroaryl is bicyclic and has from 8 to 10 ring members. Examples of bicyclic heteroaryl groups include indolyl, benzofuranyl, quinolyl, isoquinolyl indazolyl, indolinyl, isoindolyl, indolizinyl, benzamidazolyl, quinolinyl, 5,6,7,8-tetrahydroquinoline and 6,7-dihydro-5H-pyrrolo[3,2-d]pyrimidine.

The term “heteroarylalkyl” refers to an alkyl group which is attached to another moiety through an alkyl group.

### Multiple Sclerosis and Methods of Diagnosis

Multiple sclerosis (MS) is a central nervous system disease that is characterized by inflammation and loss of myelin sheaths.

Patients having MS can be identified by clinical criteria establishing a diagnosis of clinically definite MS as defined by Poser et al., Ann. Neurol. 13:227, 1983. Briefly, an individual with clinically definite MS has had two attacks and clinical evidence of either two lesions or clinical evidence of one lesion and paraclinical evidence of another, separate lesion. Definite MS may also be diagnosed by evidence of two attacks and oligoclonal bands of IgG in cerebrospinal fluid or by combination of an attack, clinical evidence of two lesions and oligoclonal band of IgG in cerebrospinal fluid. The McDonald criteria can also be used to diagnose MS. (McDonald et al., 2001, Recommended diagnostic criteria for Multiple sclerosis: guidelines from the International Panel on the Diagnosis of Multiple Sclerosis, Ann Neurol 50:121-127). The McDonald criteria include the use of MRI evidence of CNS impairment over time to be used in diagnosis of MS, in the absence of multiple clinical attacks. Effective treatment of multiple sclerosis may be evaluated in several different ways. The following parameters can be used to gauge effectiveness of treatment. Two exemplary criteria include: EDSS (extended disability status scale), and appearance of exacerbations on MRI (magnetic resonance imaging).

The EDSS is a means to grade clinical impairment due to MS (Kurtzke, Neurology 33:1444, 1983). Eight functional systems are evaluated for the type and severity of neurologic impairment. Briefly, prior to treatment, patients are evaluated for impairment in the following systems: pyramidal, cerebella, brainstem, sensory, bowel and bladder, visual, cerebral, and other. Follow-ups are conducted at defined intervals. The scale ranges from 0 (normal) to 10 (death due to MS). A decrease of one full step indicates an effective treatment (Kurtzke, Ann. Neurol. 36:573-79, 1994), while an increase of one full step will indicate the progression or worsening of disease (e.g., exacerbation). Typically patients having an EDSS score of about 6 will have moderate disability (e.g., walk with a cane), whereas patients having an EDSS score of about 7 or 8 will have severe disability (e.g., will require a wheelchair).

Exacerbations are defined as the appearance of a new symptom that is attributable to MS and accompanied by an appropriate new neurologic abnormality (IFNB MS Study Group, supra). In addition, the exacerbation must last at least 24 hours and be preceded by stability or improvement for at least 30 days. Briefly, patients are given a standard neurological examination by clinicians. Exacerbations are mild, moderate, or severe according to changes in a Neurological Rating Scale (Sipe et al., Neurology 34:1368, 1984). An annual exacerbation rate and proportion of exacerbation-free patients are determined.

Therapy can be deemed to be effective using a clinical measure if there is a statistically significant difference in the rate or proportion of exacerbation-free or relapse-free patients between the treated group and the placebo group for either of these measurements. In addition, time to first exacerbation and exacerbation duration and severity may also be measured. A measure of effectiveness as therapy in this regard is a statistically significant difference in the time to first exacerbation or duration and severity in the treated group compared to control group. An exacerbation-free or relapse-free period of greater than one year, 18 months, or 20 months is particularly noteworthy. Clinical measurements include the relapse rate in one and two-year intervals, and a change in EDSS, including time to progression from baseline of 1.0 unit on the EDSS that persists for six months. On a Kaplan-Meier curve, a delay in sustained progression of disability shows efficacy. Other criteria include a change in area and volume of T2 images on MRI, and the number and volume of lesions determined by gadolinium enhanced images.

MRI can be used to measure active lesions using gadolinium-DTPA-enhanced imaging (McDonald et al., Ann. Neurol. 36:14, 1994) or the location and extent of lesions using T2-weighted techniques. Briefly, baseline MRIs are obtained. The same imaging plane and patient position are used for each subsequent study. Positioning and imaging sequences can be chosen to maximize lesion detection and facilitate lesion tracing. The same positioning and imaging sequences can be used on subsequent studies. The presence, location and extent of MS lesions can be determined by radiologists. Areas of lesions can be outlined and summed slice by slice for total lesion area. Three analyses may be done: evidence of new lesions, rate of appearance of active lesions, percentage change in lesion area (Paty et al., Neurology 43:665, 1993). Improvement due to therapy can be established by a statistically significant improvement in an individual patient compared to baseline or in a treated group versus a placebo group.

Exemplary symptoms associated with multiple sclerosis, which can be treated with the methods described herein or managed using symptom management therapies, include: optic neuritis, diplopia, nystagmus, ocular dysmetria, internuclear opthalmoplegia, movement and sound phosphenes, afferent pupillary defect, paresis, monoparesis, paraparesis, hemiparesis, quadraparesis, plegia, paraplegia, hemiplegia, tetraplegia, quadraplegia, spasticity, dysarthria, muscle atrophy, spasms, cramps, hypotonia, clonus, myoclonus, myokymia, restless leg syndrome, footdrop, dysfunctional reflexes, paraesthesia, anaesthesia, neuralgia, neuropathic and neurogenic pain, l'hermitte's, proprioceptive dysfunction, trigeminal neuralgia, ataxia, intention tremor, dysmetria, vestibular ataxia, vertigo, speech ataxia, dystonia, dysdiadochokinesia, frequent micturation, bladder spasticity, flaccid bladder, detrusor-sphincter dyssynergia, erectile dysfunction, anorgasmy, frigidity, constipation, fecal urgency, fecal incontinence, depression, cognitive dysfunction, dementia, mood swings, emotional lability, euphoria, bipolar syndrome, anxiety, aphasia, dysphasia, fatigue, Uhthoff's symptom, gastroesophageal reflux, and sleeping disorders.

Each case of MS displays one of several patterns of presentation and subsequent course. Most commonly, MS first manifests itself as a series of attacks followed by complete or partial remissions as symptoms mysteriously lessen, only to return later after a period of stability. This is called relapsing-remitting MS (RRMS). Primary-progressive MS (PPMS) is characterized by a gradual clinical decline with no distinct remissions, although there may be temporary plateaus or minor relief from symptoms. Secondary-progressive MS (SPMS) begins with a relapsing-remitting course followed by a later primary-progressive course. Rarely, patients may have a progressive-relapsing (PRMS) course in which the disease takes a progressive path punctuated by acute attacks. PPMS, SPMS, and PRMS are sometimes lumped together and called chronic progressive MS.

A few patients experience malignant MS, defined as a swift and relentless decline resulting in significant disability or even death shortly after disease onset. This decline may be arrested or decelerated by determining the likelihood of the patient to respond to a therapy early in the therapeutic regime and switching the patient to an agent that they have the highest likelihood of responding to.

### Differentially Expressed Genes

As described in the Examples, transcriptional profiling of mouse genes was performed on C57BL/6 mice that were administered vehicle, DMF or MMF (100 mg/kg). Treated mice were sacrificed at 2, 7, or 12 hours. Tissues (liver, spleen, kidney, jejunum, cortex, hippocampus, striatum and whole blood) were collected and analyzed by transcriptional profiling on mouse Affymetrix GeneChips. Differentially expressed genes were identified by comparing DMF or MMF treated mice to matched vehicle controls and exemplary genes that were identified are provided in TABLES 1-8 below. While the experiments were performed in mice, the human homolog of the identified murine gene transcript is included in the tables. Additional genes that were identified in the study are provided in APPENDIX A, filed herewith on even date, the contents of which are herein incorporated by reference in their entirety. Additional studies are described in the Examples, and genes identified in the studies are provided in APPENDIX B, APPENDIX C, APPENDIX D, and APPENDIX E filed herewith on even date, the contents of each of which are herein incorporated by reference in their entirety.

TABLE 1
Murine Whole Blood DEG's

| | | | | | | | | | | Human |
|---|---|---|---|---|---|---|---|---|---|---|
| | | | | | | | | | | Homolog |
| | | | ENTREZ | | | | | | MMF | Entrez |
| Probe Set | Gene Title | Gene | ID | DMF 2 hr | DMF 7 hr | DMF 12 hr | MMF 2 hr | MMF 7 hr | 12 hr | ID |
| 1420937_PM_at | cleavage and | Cpsf2 | | −1.65 | | | | | | |
| | polyadenylation | | | | | | | | | |
| | specific factor 2 | | | | | | | | | |
| 1417898_PM_a_at | granzyme A | Gzma | 14938 | | | 1.92 | | | | 3001 |
| 1422089_PM_at | natural | Ncr1 | 17086 | | | 1.86 | | | | 9437 |
| | cytotoxicity | | | | | | | | | |
| | triggering | | | | | | | | | |
| | receptor 1 | | | | | | | | | |
| 1425005_PM_at | killer cell lectin- | Klrc1 | 16641 | | | 1.85 | | | | 3821 |
| | like receptor | | | | | | | | | |
| | subfamily C, | | | | | | | | | |
| | member 1 | | | | | | | | | |
| 1445399_PM_at | killer cell lectin- | Klrb1b | 80782 | | | 1.65 | | | | |
| | like receptor | | | | | | | | | |
| | subfamily B | | | | | | | | | |
| | member 1B | | | | | | | | | |
| | killer cell lectin- | Klre1 | 243665 | | | 1.85 | | | | |
| | like receptor | | | | | | | | | |
| | family E | | | | | | | | | |
| | member 1 | | | | | | | | | |
| 1418133_PM_at | B-cell | Bcl3 | | | | | −1.23 | | | |
| | leukemia/lymphoma 3 | | | | | | | | | |
| 1415943_PM_at | syndecan 1 | Sdc1 | | | | | | | −1.96 | |

TABLE 2
Murine Cortex DEGs

| | | | | | | | | | | Human |
|---|---|---|---|---|---|---|---|---|---|---|
| | | | | | | | | | | Homolog |
| | | | ENTREZ | | | | | | MMF | Entrez |
| Probe Set | Gene Title | Gene | ID | DMF 2 hr | DMF 7 hr | DMF 12 hr | MMF 2 hr | MMF 7 hr | 12 hr | ID |
| 1419086_PM_at | fibroblast | Fgfbp1 | 14181 | | 1.54 | | | 1.62 | | 9982 |
| | growth factor | | | | | | | | | |
| | binding | | | | | | | | | |
| | protein 1 | | | | | | | | | |
| 11448140_PM_at | cytokine | Ciapin1 | 109006 | −1.02 | | | | | | 57019 |
| | induced | | | | | | | | | |
| | apoptosis | | | | | | | | | |
| | inhibitor 1 | | | | | | | | | |
| 1423627_PM_at | NAD(P)H | Nqo1 | 18104 | | 1.50 | | | | | 1728 |
| | dehydrogenese, | | | | | | | | | |
| | quinone 1 | | | | | | | | | |
| 1416734_PM_at | muskelin 1, | Mkln1 | 27418 | | | | −1.85 | | | 4289 |
| | intracellular | | | | | | | | | |
| | mediator | | | | | | | | | |
| | containing | | | | | | | | | |
| | kelch motifs | | | | | | | | | |
| 1420670_PM_at | aryl | Arnt2 | 11864 | | | | −1.66 | | | 9915 |
| | hydrocarbon | | | | | | | | | |
| | receptor | | | | | | | | | |
| | nuclear | | | | | | | | | |
| | translocator 2 | | | | | | | | | |
| 1421166_PM_at | attractin | Atrn | 11990 | | | | −1.62 | | | 8455 |
| 1421339_PM_at | exostoses | Extl3 | 54616 | | | | −1.57 | | | 2137 |
| | (multiple)- | | | | | | | | | |
| | like 3 | | | | | | | | | |
| 1423594_PM_a_at | endothelin | Ednrb | 13618 | | | | −1.53 | | | 1910 |
| | receptor type B | | | | | | | | | |
| 1429607_PM_at | trafficking | Trak2 | 70827 | | | | −1.57 | | | 66008 |
| | protein, | | | | | | | | | |
| | kinesin | | | | | | | | | |
| | binding 2 | | | | | | | | | |
| 1429992_PM_at | spermatogenesis | Speer4b | 73526 | | | | −1.60 | | | NA |
| | associated | | | | | | | | | |
| | glutamate | | | | | | | | | |
| | (E)-rich | | | | | | | | | |
| | protein 4b | | | | | | | | | |
| 1430827_PM_a_at | PTK2 protein | Ptk2 | 14083 | | | | −1.53 | | | 5747 |
| | tyrosine | | | | | | | | | |
| | kinase 2 | | | | | | | | | |
| 1438108_PM_at | pleckstrin | Plekhm3 | 241075 | | | | −1.50 | | | 389072 |
| | homology | | | | | | | | | |
| | domain | | | | | | | | | |
| | containing, | | | | | | | | | |
| | family M, | | | | | | | | | |
| | member 3 | | | | | | | | | |
| 1442277_PM_at | choline | Chka | 12660 | | | | −1.53 | | | 1119 |
| | kinase alpha | | | | | | | | | |
| 1443123_PM_at | tetratricopeptide | Tanc2 | 77097 | | | | −1.72 | | | 26115 |
| | repeat, | | | | | | | | | |
| | ankyrin | | | | | | | | | |
| | repeat and | | | | | | | | | |
| | coiled-coil | | | | | | | | | |
| | containing 2 | | | | | | | | | |
| 1448458_PM_at | topoisomerase | Top2b | 21974 | | | | −1.51 | | | 7155 |
| | (DNA) II | | | | | | | | | |
| | beta | | | | | | | | | |
| 1456070_PM_at | protein | Ptprg | 19270 | | | | −1.83 | | | 5793 |
| | tyrosine | | | | | | | | | |
| | phosphatase, | | | | | | | | | |
| | receptor | | | | | | | | | |
| | type, G | | | | | | | | | |
| 1419435_PM_at | aldehyde | Aox1 | 11761 | | | | | 1.65 | 1.54 | 316 |
| | oxidase 1 | | | | | | | | | |

TABLE 3
Murine Hippocampus DEGs

| | | | ENTREZ | | | DMF | | | MMF |
|---|---|---|---|---|---|---|---|---|---|
| Probe Set | Gene Title | Gene | ID | DMF 2 hr | DMF 7 hr | 12 hr | MMF 2 hr | MMF 7 hr | 12 hr |
| 1458305_PM_at | transmembrane | Tmtc3 | 237500 | | −2.38 | | | −2.40 | |
| | and | | | | | | | | |
| | tetratricopeptide | | | | | | | | |
| | repeat | | | | | | | | |
| | containing 3 | | | | | | | | |
| 1417975_PM_at | karyopherin | Kpna4 | 16649 | | | | −1.51 | | |
| | (importin) | | | | | | | | |
| | alpha 4 | | | | | | | | |
| 1425077_PM_at | DnaJ (Hsp40) | Dnajc18 | 76594 | | | | −1.58 | | |
| | homolog, | | | | | | | | |
| | subfamily C, | | | | | | | | |
| | member 18 | | | | | | | | |
| 1426029_PM_a_at | nuclear factor | Nfat5 | 54446 | | | | −1.51 | | |
| | of activated T- | | | | | | | | |
| | cells 5 | | | | | | | | |
| 1434407_PM_at | SLIT-ROBO | Srgap2 | 14270 | | | | −1.50 | | |
| | Rho GTPase | | | | | | | | |
| | activating | | | | | | | | |
| | protein 2 | | | | | | | | |
| 1438085_PM_at | HEAT repeat | Heatr5b | 320473 | | | | −1.56 | | |
| | containing 5B | | | | | | | | |
| 1458421_PM_at | potassium | Kcnq3 | 110862 | | | | −1.50 | | |
| | voltage-gated | | | | | | | | |
| | channel, | | | | | | | | |
| | subfamily Q, | | | | | | | | |
| | member 3 | | | | | | | | |

TABLE 4
Murine Striatum DEGs

| | | | | | | | | | | Human |
|---|---|---|---|---|---|---|---|---|---|---|
| | | | | | | | | | | Homolog |
| Probe | | | ENTREZ | | | DMF | | MMF | MMF | Entrez |
| Set | Gene Title | Gene | ID | DMF 2 hr | DMF 7 hr | 12 hr | MMF 2 hr | 7 hr | 12 hr | ID |
| 1419435_PM_at | aldehyde | Aox1 | 11761 | | 1.64 | | | 1.95 | | 316 |
| | oxidase 1 | | | | | | | | | |
| 1428320_PM_at | KDM3B lysine | Kdm3b | 277250 | −1.54 | | | −1.56 | | | 51780 |
| | (K)-specific | | | | | | | | | |
| | demethylase | | | | | | | | | |
| | 3B | | | | | | | | | |
| 1434030_PM_at | GTP-binding | Gtpbp10 | 207704 | −1.52 | | | | | | 85865 |
| | protein 10 | | | | | | | | | |
| | (putative) | | | | | | | | | |
| 1456070_PM_at | protein | Ptprg | 19270 | −1.54 | | | | | | 5793 |
| | tyrosine | | | | | | | | | |
| | phosphatase, | | | | | | | | | |
| | receptor type, G | | | | | | | | | |
| 1416734_PM_at | muskelin 1, | Mkln1 | 27418 | −1.97 | | | | | | 4289 |
| | intracellular | | | | | | | | | |
| | mediator | | | | | | | | | |
| | containing | | | | | | | | | |
| | ketch motifs | | | | | | | | | |
| 1438236_PM_at | nuclear factor | Nfia | 18027 | | | −1.53 | | | | 4774 |
| | I/A | | | | | | | | | |
| 1422748_PM_at | zinc finger E- | Zeb2 | 24136 | | | −1.66 | | | | 9839 |
| | box binding | | | | | | | | | |
| | homeobox 2 | | | | | | | | | |
| 1427125_PM_s_at | leucine rich | Lrrc41 | 230654 | | | | −1.54 | | | 10489 |
| | repeat | | | | | | | | | |
| | containing 41 | | | | | | | | | |
| 1448458_PM_at | topoisomerase | Top2b | 21974 | | | | −1.56 | | | 7155 |
| | (DNA) II beta | | | | | | | | | |
| 1449616_PM_s_at | golgi | Golga3 | 269682 | | | | −1.63 | | | 2802 |
| | autoantigen, | | | | | | | | | |
| | golgin | | | | | | | | | |
| | subfamily a, 3 | | | | | | | | | |
| 1422556_PM_at | guanine | Gna13 | 14674 | | | | −1.63 | | | 10672 |
| | nucleotide | | | | | | | | | |
| | binding | | | | | | | | | |
| | protein, alpha | | | | | | | | | |
| | 13 | | | | | | | | | |
| 1421616_PM_at | glutamate | Grin2a | 14811 | | | | −1.78 | | | 2903 |
| | receptor, | | | | | | | | | |
| | ionotropic, | | | | | | | | | |
| | NMDA2A | | | | | | | | | |
| | (epsilon 1) | | | | | | | | | |
| 1428676_PM_at | transmembrane | Tmprss6 | 71753 | | | | | | 1.68 | 164656 |
| | serine protease 6 | | | | | | | | | |
| 1448807_PM_at | histamine | Hrh3 | 99296 | | | | | | 1.59 | 11255 |
| | receptor H3 | | | | | | | | | |
| 1437760_PM_at | UDP-N-acetyl- | Galnt12 | 230145 | | | | | | 1.50 | 79695 |
| | alpha-D- | | | | | | | | | |
| | galactosamine: | | | | | | | | | |
| | polypeptide N- | | | | | | | | | |
| | acetylgalactosaminyltransferase | | | | | | | | | |
| | 12 | | | | | | | | | |
| 1435272_PM_at | inositol 1,4,5- | Itpkb | 320404 | | | | | | −1.56 | 3707 |
| | trisphosphate | | | | | | | | | |
| | 3-kinase B | | | | | | | | | |
| 1455123_PM_at | suppression of | St18 | 240690 | | | | | | −1.69 | 9705 |
| | tumorigenicity | | | | | | | | | |
| | 18 | | | | | | | | | |
| 1457257_PM_x_at | poliovirus | Pvrl3 | 58998 | | | | | | −1.80 | 25945 |
| | receptor- | | | | | | | | | |
| | related 3 | | | | | | | | | |
| 1437785_PM_at | a disintegrin- | Adamts9 | 101401 | | | | | | −2.58 | 56999 |
| | like and | | | | | | | | | |
| | metallopeptidase | | | | | | | | | |
| | (reprolysin | | | | | | | | | |
| | type) with | | | | | | | | | |
| | thrombospondin | | | | | | | | | |
| | type 1 motif, 9 | | | | | | | | | |

TABLE 5a
Murine Jejunum DMF-specific DEGs

| | | | | | | | Human |
|---|---|---|---|---|---|---|---|
| | | | | | | | Homolog |
| | | | ENTREZ | DMF | DMF | DMF | Entrez |
| Probe Set | Gene Title | Gene | ID | 2 hr | 7 hr | 12 hr | ID |
| 1440230_PM_at | tsukushin | Tsku | 244152 | 1.61 | | | 25987 |
| 1453421_PM_at | serine racemase | Srr | 27364 | 1.99 | | | 63826 |
| 1428834_PM_at | dual specificity | Dusp4 | 319520 | 1.51 | | | 1846 |
| | phosphatase 4 | | | | | | |
| 1429950_PM_at | unc-5 homolog C (C. elegans)- | Unc5cl | 76589 | −1.53 | | | 222643 |
| | like | | | | | | |
| 1420393_PM_at | nitric oxide synthase | Nos2 | 18126 | −2.39 | | | 4843 |
| | 2, inducible | | | | | | |
| 1442923_PM_at | PTK6 protein tyrosine | Ptk6 | 20459 | −2.42 | | | 5753 |
| | kinase 6 | | | | | | |
| 1426501_PM_a_at | TRAF-interacting | Tifa | 211550 | −2.43 | | | 92610 |
| | protein with forkhead- | | | | | | |
| | associated domain | | | | | | |
| 1428397_PM_at | UDP- | B3galt5 | 93961 | −2.77 | | | 10317 |
| | Gal:betaGlcNAc beta | | | | | | |
| | 1,3- | | | | | | |
| | galactosyltransferase, | | | | | | |
| | polypeptide 5 | | | | | | |
| 1456611_PM_at | family with sequence | Fam13a | 58909 | 1.54 | | | 10144 |
| | similarity 13, member A | | | | | | |

TABLE 5b
Murine Jejunum MMF-specific DEGs

| | | | | | | | | | | Human |
|---|---|---|---|---|---|---|---|---|---|---|
| | | | | | | | | | | Homolog |
| | | | ENTREZ | | | | | | MMF | Entrez |
| Probe Set | Gene Title | Gene | ID | DMF 2 hr | DMF 7 hr | DMF 12 hr | MMF 2 hr | MMF 7 hr | 12 hr | ID |
| 11440882_PM_at | low density | Lrp8 | 16975 | | | | 2.23 | | | 7804 |
| | lipoprotein | | | | | | | | | |
| | receptor- | | | | | | | | | |
| | related protein | | | | | | | | | |
| | 8, | | | | | | | | | |
| | apolipoproteine | | | | | | | | | |
| | receptor | | | | | | | | | |
| 1448239_PM_at | heme | Hmox1 | 15368 | | | | 1.81 | | | 3162 |
| | oxygenase | | | | | | | | | |
| | (decycling) 1 | | | | | | | | | |
| 1436605_PM_at | transketolase | Tkt | 21881 | | | | 1.73 | | | 7086 |
| 1422645_PM_at | hemochromatosis | Hfe | 15216 | | | | 1.73 | | | 3077 |
| 1426600_PM_at | solute carrier | Slc2a1 | 20525 | | | | 1.68 | | | 6513 |
| | family 2 | | | | | | | | | |
| | (facilitated | | | | | | | | | |
| | glucose | | | | | | | | | |
| | transporter), | | | | | | | | | |
| | member 1 | | | | | | | | | |
| 1438953_PM_at | c-fos induced | Figf | 14205 | | | | 1.63 | | | 2277 |
| | growth factor | | | | | | | | | |
| 11450410_PM_a_at | solute carrier | Slc48a1 | 67739 | | | | 1.62 | | | 55652 |
| | family 48 | | | | | | | | | |
| | (heme | | | | | | | | | |
| | transporter), | | | | | | | | | |
| | member 1 | | | | | | | | | |
| 1452837_PM_at | lipin 2 | Lpin2 | 64898 | | | | 1.61 | | | 9663 |
| 1424181_PM_at | septin 6 | 6-Sep | 56526 | | | | 1.56 | | | 23157 |
| 1449078_PM_at | ST3 beta- | St3gal6 | 54613 | | | | 1.52 | | | 10402 |
| | galactoside | | | | | | | | | |
| | alpha-2,3- | | | | | | | | | |
| | sialyltransferase 6 | | | | | | | | | |
| 1416418_PM_at | gamma- | Gabarapl1 | 57436 | | | | 1.52 | | | 23710 |
| | aminobutyric | | | | | | | | | |
| | acid (GABA) | | | | | | | | | |
| | A receptor- | | | | | | | | | |
| | associated | | | | | | | | | |
| | protein-like 1 | | | | | | | | | |
| 1423635_PM_at | bone | Bmp2 | 12156 | | | | −1.55 | | | 650 |
| | morphogenetic | | | | | | | | | |
| | protein 2 | | | | | | | | | |
| 1421886_PM_at | son of | Sos1 | 20662 | | | | −1.57 | | | 6654 |
| | sevenless | | | | | | | | | |
| | homolog 1 | | | | | | | | | |
| | (Drosophila) | | | | | | | | | |
| 1452834_PM_at | proline rich 5 | Prr5l | 72446 | | | | −1.64 | | | 79899 |
| | like | | | | | | | | | |
| 1433699_PM_at | tumor | Tnfaip3 | 21929 | | | | −1.67 | | | 7128 |
| | necrosis | | | | | | | | | |
| | factor, alpha- | | | | | | | | | |
| | induced | | | | | | | | | |
| | protein 3 | | | | | | | | | |
| 1450165_PM_at | schlafen 2 | Slfn2 | 20556 | | | | −1.82 | | | NA |
| 1416632_PM_at | malic enzyme | Me1 | 17436 | | | | | 2.01 | | 4199 |
| | 1, NADP(+)- | | | | | | | | | |
| | dependent, | | | | | | | | | |
| | cytosolic | | | | | | | | | |
| 1419072_PM_at | glutathione S- | Gstm7 | 68312 | | | | | 1.97 | | 2946 |
| | transferase, | | | | | | | | | |
| | mu 7 | | | | | | | | | |
| 1451385_PM_at | family with | Fam162a | 70186 | | | | | 1.67 | | 26355 |
| | sequence | | | | | | | | | |
| | similarity 162, | | | | | | | | | |
| | member A | | | | | | | | | |
| 1451095_PM_at | asparagine | Asns | 27053 | | | | | 1.60 | | 440 |
| | synthetase | | | | | | | | | |
| 1419442_PM_at | matrilin 2 | Matn2 | 17181 | | | | | 1.59 | | 4147 |
| 1423706_PM_a_at | phosphogluconate | Pgd | 110208 | | | | | 1.55 | | 5226 |
| | dehydrogenase | | | | | | | | | |
| 1450109_PM_s_at | ATP-binding | Abcc2 | 12780 | | | | | 1.51 | | 1244 |
| | cassette, sub- | | | | | | | | | |
| | family C | | | | | | | | | |
| | (CFTR/MRP), | | | | | | | | | |
| | member 2 | | | | | | | | | |
| 1428960_PM_at | enkurin, | Enkur | 71233 | | | | | −1.56 | | 219670 |
| | TRPC channel | | | | | | | | | |
| | interacting | | | | | | | | | |
| | protein | | | | | | | | | |
| 1418580_PM_at | receptor | Rtp4 | 67775 | | | | | −1.61 | | 64108 |
| | transporter | | | | | | | | | |
| | protein 4 | | | | | | | | | |
| 1435529_PM_at | predicted gene | Gm14446 | 667373 | | | | | −1.65 | | 3434 |
| | 14446 | | | | | | | | | |
| 1437960_PM_at | calpain 13 | Capn13 | 38112 | | | | | −1.75 | | 92291 |
| 1436058_PM_at | radical S- | Rsad2 | 58185 | | | | | −1.81 | | 91543 |
| | adenosyl | | | | | | | | | |
| | methionine | | | | | | | | | |
| | domain | | | | | | | | | |
| | containing 2 | | | | | | | | | |
| 1450783_PM_at | interferon- | Ifit1 | 15957 | | | | | −1.95 | | 439996 |
| | induced | | | | | | | | | |
| | protein with | | | | | | | | | |
| | tetratricopeptide | | | | | | | | | |
| | repeats 1 | | | | | | | | | |
| 1422438_PM_at | epoxide | Ephx1 | 13849 | | | | | | 2.17 | 2052 |
| | hydrolase 1, | | | | | | | | | |
| | microsomal | | | | | | | | | |
| 1434458_PM_at | follistatin | Fst | 14313 | | | | | | 1.94 | 10468 |
| 1422000_PM_at | aldo-keto | Akr1c12 | 622402 | | | | | | 1.52 | 622402 |
| | reductase | | | | | | | | | |
| | family 1, | | | | | | | | | |
| | member C12 | | | | | | | | | |
| 1417991_PM_at | deiodinase, | Dio1 | 13370 | | | | | | −1.51 | 1733 |
| | iodothyronine, | | | | | | | | | |
| | type I | | | | | | | | | |
| 1451642_PM_at | kinesin family | Kif1b | 16561 | | | | | | −1.51 | 23095 |
| | member 1B | | | | | | | | | |
| 1457554_PM_at | apolipoprotein B | Apob | 238055 | | | | | | −1.54 | 338 |

TABLE 6
Murine Kidney DMF specific DEGs

| | | | | | | | Human |
|---|---|---|---|---|---|---|---|
| | | | | DMF | DMF | DMF | Homolog |
| Probe Set | Gene Title | Gene | ENTREZ ID | 2 hr | 7 hr | 12 hr | Entrez ID |
| 1436521_PM_at | solute carrier | Slc36a2 | 246049 | 4.63 | | | 153201 |
| | family 36 | | | | | | |
| | (proton/amino acid | | | | | | |
| | symporter), | | | | | | |
| | member 2 | | | | | | |
| 1422612_PM_at | hexokinase 2 | Hk2 | 15277 | 2.72 | | | 3099 |
| 1438664_PM_at | protein kinase, | Prkar2b | 19088 | 2.43 | | | 5577 |
| | cAMP dependent | | | | | | |
| | regulatory, type II | | | | | | |
| | beta | | | | | | |
| 1434893_PM_at | ATPase, Na+/K+ | Atp1a2 | 98660 | 2.25 | | | 477 |
| | transporting, alpha | | | | | | |
| | 2 polypeptide | | | | | | |
| 1423405_PM_at | tissue inhibitor of | Timp4 | 110595 | 1.91 | | | 7079 |
| | metalloproteinase 4 | | | | | | |
| 1453596_PM_at | inhibitor of DNA | Id2 | 15902 | 1.83 | | | 3398 |
| | binding 2 | | | | | | |
| 1456041_PM_at | sorting nexin 16 | Snx16 | 74718 | 1.75 | | | 64089 |
| 1436366_PM_at | protein | Ppp1r15b | 108954 | 1.72 | | | 84919 |
| | phosphatase 1, | | | | | | |
| | regulatory | | | | | | |
| | (inhibitor) subunit | | | | | | |
| | 15b | | | | | | |
| 1448170_PM_at | seven in absentia 2 | Siah2 | 20439 | 1.71 | | | 6478 |
| 1441190_PM_at | actin related | Arpc5l | 74192 | 1.70 | | | 81873 |
| | protein 2/3 | | | | | | |
| | complex, subunit | | | | | | |
| | 5-like | | | | | | |
| 1455657_PM_at | SMG1 homolog, | Smg1 | 233789 | 1.69 | | | 23049 |
| | phosphatidylinositol | | | | | | |
| | 3-kinase-related | | | | | | |
| | kinase (C. elegans) | | | | | | |
| 1418203_PM_at | phorbol-12- | Pmaip1 | 58801 | 1.68 | | | NA |
| | myristate-13- | | | | | | |
| | acetate-induced | | | | | | |
| | protein 1 | | | | | | |
| 1433944_PM_at | HECT domain | Hectd2 | 226098 | 1.67 | | | 143279 |
| | containing 2 | | | | | | |
| 1423170_PM_at | TAF7 RNA | Taf7 | 24074 | 1.66 | | | 6879 |
| | polymerase II, | | | | | | |
| | TATA box binding | | | | | | |
| | protein (TBP)- | | | | | | |
| | associated factor | | | | | | |
| 1452394_PM_at | cysteinyl-tRNA | Cars | 27267 | 1.63 | | | 833 |
| | synthetase | | | | | | |
| 1441546_PM_at | Membrane protein, | Mpp6 | 56524 | 1.62 | | | 51678 |
| | palmitoylated 6 | | | | | | |
| | (MAGUK p55 | | | | | | |
| | subfamily member 6) | | | | | | |
| 1441955_PM_s_at | polyadenylate | Paip1 | 218693 | 1.61 | | | 10605 |
| | binding protein- | | | | | | |
| | interacting protein 1 | | | | | | |
| 1438578_PM_a_at | BTB (POZ) | Btbd10 | 68815 | 1.60 | | | 84280 |
| | domain containing 10 | | | | | | |
| 1418025_PM_at | basic helix-loop- | Bhlhe40 | 20893 | 1.59 | | | 8553 |
| | helix family, | | | | | | |
| | member e40 | | | | | | |
| 1459585_PM_at | growth arrest | Gas6 | 14456 | 1.59 | | | 2621 |
| | specific 6 | | | | | | |
| 1417479_PM_at | protein | Ppp2r3c | 59032 | 1.59 | | | 55012 |
| | phosphatase 2, | | | | | | |
| | regulatory subunit | | | | | | |
| | B″, gamma | | | | | | |
| 1455185_PM_s_at | PHD finger protein 16 | Phf16 | 382207 | 1.58 | | | 9767 |
| 1436871_PM_at | serine/arginine-rich | Srsf7 | 225027 | 1.57 | | | 6432 |
| | splicing factor 7 | | | | | | |
| 1452094_PM_at | procollagen- | P4ha1 | 18451 | 1.56 | | | 5033 |
| | proline, | | | | | | |
| | 2-oxoglutarate | | | | | | |
| | 4-dioxygenase | | | | | | |
| | (proline | | | | | | |
| | 4-hydroxylase), alpha | | | | | | |
| | 1 polypeptide | | | | | | |
| 1443116_PM_at | proteasome (prosome, | Psme4 | 103554 | 1.56 | | | 23198 |
| | macropain) | | | | | | |
| | activator subunit 4 | | | | | | |
| 1424380_PM_at | vacuolar protein | Vps37b | 330192 | 1.56 | | | 79720 |
| | sorting 37B (yeast) | | | | | | |
| 1456810_PM_at | vacuolar protein | Vps54 | 245944 | 1.54 | | | 51542 |
| | sorting 54 (yeast) | | | | | | |
| 1452218_PM_at | coiled-coil domain | Ccdc117 | 104479 | 1.54 | | | 150275 |
| | containing 117 | | | | | | |
| 1435904_PM_at | eukaryotic translation | Eif2c3 | 214150 | 1.53 | | | 192669 |
| | initiation factor 2C, 3 | | | | | | |
| 1442071_PM_at | ATP-binding | Abce1 | 24015 | 1.53 | | | 6059 |
| | cassette, sub- | | | | | | |
| | family E (OABP), | | | | | | |
| | member 1 | | | | | | |
| 1440346_PM_at | KDM1 lysine (K)- | Kdm6b | 216850 | 1.52 | | | 23135 |
| | specific | | | | | | |
| | demethylase 6B | | | | | | |
| 1423549_PM_at | solute carrier | Slc1a4 | 55963 | 1.52 | | | 6509 |
| | family 1 | | | | | | |
| | (glutamate/neutral | | | | | | |
| | amino acid | | | | | | |
| | transporter), | | | | | | |
| | member 4 | | | | | | |
| 1435912_PM_at | UBX domain | Ubxn7 | 224111 | 1.51 | | | 26043 |
| | protein 7 | | | | | | |
| 1423605_PM_a_at | transformed mouse | Mdm2 | 17246 | 1.51 | | | 4193 |
| | 3T3 cell double | | | | | | |
| | minute 2 | | | | | | |
| 1425656_PM_a_at | brain-specific | Baiap2 | 108100 | 1.51 | | | 10458 |
| | angiogenesis | | | | | | |
| | inhibitor 1- | | | | | | |
| | associated protein 2 | | | | | | |
| 1425287_PM_at | zinc finger protein | Zfp189 | 230162 | 1.51 | | | 7743 |
| | 189 | | | | | | |
| 1455904_PM_at | growth arrest | Gas5 /// | 100217446 /// | 1.51 | | | NA // NA |
| | specific 5 /// small | Snord47 | 14455 | | | | |
| | nucleolar RNA, | | | | | | |
| | C/D box 47 | | | | | | |
| 1438535_PM_at | pleckstrin | Phip | 83946 | 1.50 | | | 55023 |
| | homology domain | | | | | | |
| | interacting protein | | | | | | |
| 1419140_PM_at | activin receptor IIB | Acvr2b | 11481 | −1.51 | | | 93 |
| 1428975_PM_at | sushi domain | Susd3 | 66329 | −1.51 | | | 203328 |
| | containing 3 | | | | | | |
| 1437231_PM_at | SLIT and NTRK- | Slitrk6 | 239250 | −1.52 | | | 84189 |
| | like family, | | | | | | |
| | member 6 | | | | | | |
| 1452962_PM_at | transmembrane | Tmem25 | 71687 | −1.52 | | | 84866 |
| | protein 25 | | | | | | |
| 1438784_PM_at | B-cell | Bcl11b | 58208 | −1.52 | | | 64919 |
| | leukemia/lymphoma 11B | | | | | | |
| 1427369_PM_at | NLR family, pyrin | Nlrp6 | 101613 | −1.52 | | | 171389 |
| | domain containing 6 | | | | | | |
| 1455087_PM_at | DNA segment, Chr | D7Ertd715e | 52480 | −1.53 | | | NA |
| | 7, ERATO Doi | | | | | | |
| | 715, expressed | | | | | | |
| 1449813_PM_at | zinc finger protein 30 | Zfp30 | 22693 | −1.55 | | | 22835 |
| 1451692_PM_at | transmembrane and | Tmco6 | 71983 | −1.57 | | | 55374 |
| | coiled-coil domains 6 | | | | | | |
| 1419051_PM_at | OVO homolog-like | Ovol1 | 18426 | −1.60 | | | 5017 |
| | 1 (Drosophila) | | | | | | |
| 1424213_PM_at | UbiA | Ubiad1 | 71707 | −1.61 | | | 29914 |
| | prenyltransferase | | | | | | |
| | domain containing 1 | | | | | | |
| 1441198_PM_at | zinc finger protein 39 | Zfp39 | 22698 | −1.64 | | | 100131827 |
| 1446303_PM_at | insulin-like growth | Igf1r | 16001 | −1.66 | | | 3480 |
| | factor I receptor | | | | | | |
| 1429456_PM_a_at | polymerase (RNA) | Polr3e | 26939 | −1.69 | | | 55718 |
| | III (DNA directed) | | | | | | |
| | polypeptide E | | | | | | |
| 1457548_PM_at | A disintegrin-like and | Adamts6 | 108154 | −1.83 | | | 11174 |
| | metallopeptidase | | | | | | |
| | (reprolysin type) | | | | | | |
| | with thrombospondin | | | | | | |
| | type 1 motif, 6 | | | | | | |
| 1449981_PM_a_at | N-acetyltransferase | Nat2 | 17961 | −2.01 | | | 9 |
| | 2 (arylamine | | | | | | |
| | N-acetyltransferase) | | | | | | |
| 1452975_PM_at | alanine-glyoxylate | Agxt2l1 | 71760 | −2.10 | | | 64850 |
| | aminotransferase | | | | | | |
| | 2-like 1 | | | | | | |
| 1427056_PM_at | a disintegrin-like and | Adamts15 | 235130 | −2.26 | | | 170689 |
| | metallopeptidase | | | | | | |
| | (reprolysin type) | | | | | | |
| | with thrombospondin | | | | | | |
| | type 1 motif, 15 | | | | | | |
| 1427372_PM_at | cytochrome P450, | Cyp27b1 | 13115 | −3.22 | | | 1594 |
| | family 27, | | | | | | |
| | subfamily b, | | | | | | |
| | polypeptide 1 | | | | | | |
| 1427094_PM_at | polymerase (DNA | Pole2 | 18974 | | 1.75 | | 5427 |
| | directed), epsilon 2 | | | | | | |
| | (p59 subunit) | | | | | | |
| 1440899_PM_at | flavin containing | Fmo5 | 14263 | | −1.51 | | 2330 |
| | monooxygenase 5 | | | | | | |
| 1460196_PM_at | carbonyl reductase 1 | Cbr1 | 12408 | | | 1.80 | 873 |
| 1421108_PM_at | camello-like 2 | Cml2 | 93673 | | | 1.60 | NA |
| 1421603_PM_a_at | carcinoembryonic | Ceacam2 | 26367 | | | −1.68 | NA |
| | antigen-related cell | | | | | | |
| | adhesion molecule 2 | | | | | | |

TABLE 7
Murine Liver DEGs

| | | | | | | | | | | Human |
|---|---|---|---|---|---|---|---|---|---|---|
| | | | | DMF | DMF | DMF | MMF | MMF | MMF | Homolog |
| Probe Set | Gene Title | Gene | ENTREZ ID | 2 hr | 7 hr | 12 hr | 2 hr | 7 hr | 12 hr | Entrez ID |
| 1448894_PM_at | aldo-keto | Akr1b8 | 14187 | 2.18 | 1.58 | | 2.27 | 1.75 | | 57016 |
| | reductase | | | | | | | | | |
| | family 1, | | | | | | | | | |
| | member B8 | | | | | | | | | |
| 1419627_PM_s_at | C-type lectin | Clec4n | 56620 | 2.34 | 2.01 | | | 2.40 | | 93978 |
| | domain family | | | | | | | | | |
| | 4, member n | | | | | | | | | |
| 1419942_PM_at | Sulfiredoxin 1 | Srxn1 | 76650 | 1.69 | | | 1.68 | 1.52 | | 140809 |
| | homolog | | | | | | | | | |
| | (S. cerevisiae) | | | | | | | | | |
| 1417801_PM_a_at | PTPRF interacting | Ppfibp2 | 19024 | 2.38 | | | 2.55 | | | 8495 |
| | protein, binding | | | | | | | | | |
| | protein 2 | | | | | | | | | |
| | (liprin beta 2) | | | | | | | | | |
| 1448239_PM_at | heme oxygenase | Hmox1 | 15368 | 2.37 | | | 2.41 | | | 3162 |
| | (decycling) 1 | | | | | | | | | |
| 1420804_PM_s_at | C-type lectin | Clec4d | 17474 | 2.30 | | | 2.05 | | | 338339 |
| | domain family | | | | | | | | | |
| | 4, member d | | | | | | | | | |
| 1438953_PM_at | c-fos induced | Figf | 14205 | 2.23 | | | 2.25 | | | 2277 |
| | growth factor | | | | | | | | | |
| 1452233_PM_at | ATP-binding | Abcc1 | 17250 | 1.75 | | | 1.86 | | | 4363 |
| | cassette, sub- | | | | | | | | | |
| | family C | | | | | | | | | |
| | (CFTR/MRP), | | | | | | | | | |
| | member 1 | | | | | | | | | |
| 1436771_PM_x_at | phosphogluconate | Pgd | 110208 | 1.59 | | | 1.61 | | | 5226 |
| | dehydrogenase | | | | | | | | | |
| 1423627_PM_at | NAD(P)H | Nqo1 | 18104 | | 1.81 | | | 2.20 | | 1728 |
| | dehydrogenase, | | | | | | | | | |
| | quinone 1 | | | | | | | | | |
| 1450759_PM_at | bone | Bmp6 | 12161 | | 1.57 | | | 1.65 | | 654 |
| | morphogenetic | | | | | | | | | |
| | protein 6 | | | | | | | | | |
| 1435495_PM_at | adenosine A1 | Adora1 | 11539 | | 1.50 | | | | | 134 |
| | receptor | | | | | | | | | |
| 1424835_PM_at | glutathione | Gstm4 | 14865 | | | 1.12 | | | | 2948 |
| | S-transferase, | | | | | | | | | |
| | mu 4 | | | | | | | | | |
| 1424296_PM_at | glutamate-cysteine | Gclc | 14629 | | | | 1.72 | | | 2729 |
| | ligase, catalytic | | | | | | | | | |
| | subunit | | | | | | | | | |
| 1422573_PM_at | adenosine | Ampd3 | 11717 | | | | 1.70 | | | 272 |
| | monophosphate | | | | | | | | | |
| | deaminase 3 | | | | | | | | | |
| 1455016_PM_at | PRP38 pre-mRNA | Prpf38b | 66921 | | | | −1.53 | | | 55119 |
| | processing | | | | | | | | | |
| | factor 38 | | | | | | | | | |
| | (yeast) | | | | | | | | | |
| | domain | | | | | | | | | |
| | containing B | | | | | | | | | |
| 1452247_PM_at | fragile X | Fxr1 | 14359 | | | | −1.53 | | | 8087 |
| | mental | | | | | | | | | |
| | retardation | | | | | | | | | |
| | gene 1, | | | | | | | | | |
| | autosomal | | | | | | | | | |
| | homolog | | | | | | | | | |
| 1447019_PM_at | cytidine | Cmah | 12763 | | | | −1.55 | | | NA |
| | monophospho-N- | | | | | | | | | |
| | acetylneuraminic | | | | | | | | | |
| | acid | | | | | | | | | |
| | hydroxylase | | | | | | | | | |
| 1448348_PM_at | cell cycle | Caprin1 | 53872 | | | | −1.56 | | | 4076 |
| | associated | | | | | | | | | |
| | protein 1 | | | | | | | | | |
| 1419038_PM_a_at | casein kinase | Csnk2a1 | 12995 | | | | −1.62 | | | 1457 |
| | 2, alpha 1 | | | | | | | | | |
| | polypeptide | | | | | | | | | |
| 1440091_PM_at | Meis homeobox 2 | Meis2 | 17536 | | | | −1.67 | | | 4212 |
| 1416734_PM_at | muskelin 1, | Mkln1 | 27418 | | | | −1.69 | | | 4289 |
| | intracellular | | | | | | | | | |
| | mediator | | | | | | | | | |
| | containing | | | | | | | | | |
| | kelch motifs | | | | | | | | | |
| 1453908_PM_at | protein | Ptprb | 19263 | | | | −1.76 | | | 5787 |
| | tyrosine | | | | | | | | | |
| | phosphatase, | | | | | | | | | |
| | receptor type, B | | | | | | | | | |
| 1456070_PM_at | protein | Ptprg | 19270 | | | | −1.85 | | | 5793 |
| | tyrosine | | | | | | | | | |
| | phosphatase, | | | | | | | | | |
| | receptor type, G | | | | | | | | | |
| 1449498_PM_at | macrophage | Marco | 17167 | | | | | 2.44 | | 8685 |
| | receptor with | | | | | | | | | |
| | collagenous | | | | | | | | | |
| | structure | | | | | | | | | |
| 1427357_PM_at | cytidine | Cda | 72269 | | | | | 1.92 | | 978 |
| | deaminase | | | | | | | | | |
| 1448354_PM_at | glucose-6- | G6pdx | 14381 | | | | | 1.61 | | 2539 |
| | phosphate | | | | | | | | | |
| | dehydrogenase | | | | | | | | | |
| | X-linked | | | | | | | | | |
| 1435040_PM_at | interleukin-1 | Irak3 | 73914 | | | | | 1.53 | | 11213 |
| | receptor- | | | | | | | | | |
| | associated | | | | | | | | | |
| | kinase 3 | | | | | | | | | |
| 1429001_PM_at | pirin | Pir | 69656 | | | | | 1.50 | | 8544 |
| 1445815_PM_at | frizzled | Fzd8 | 14370 | | | | | −1.81 | | 8325 |
| | homolog 8 | | | | | | | | | |
| | (Drosophila) | | | | | | | | | |
| 1434582_PM_at | ELKS/RAB6- | Erc2 | 238988 | | | | | −1.95 | | 26059 |
| | interacting/ | | | | | | | | | |
| | CAST family | | | | | | | | | |
| | member 2 | | | | | | | | | |
| 1419669_PM_at | proteinase 3 | Prtn3 | 19152 | | | | | | 4.46 | 5657 |
| 1431214_PM_at | predicted gene | Gm3579 | 100041932 | | | | | | 2.74 | NA |
| | 3579 | | | | | | | | | |
| 1417441_PM_at | DnaJ (Hsp40) | Dnajc12 | 30045 | | | | | | 2.53 | 56521 |
| | homolog, | | | | | | | | | |
| | subfamily C, | | | | | | | | | |
| | member 12 | | | | | | | | | |
| 1428154_PM_s_at | phosphatidic | Ppapdc1b | 71910 | | | | | | 1.87 | 84513 |
| | acid | | | | | | | | | |
| | phosphatase | | | | | | | | | |
| | type 2 domain | | | | | | | | | |
| | containing 1B | | | | | | | | | |
| 1444176_PM_at | ATPase, H+ | Atp6v0d2 | 242341 | | | | | | 1.84 | 245972 |
| | transporting, | | | | | | | | | |
| | lysosomal V0 | | | | | | | | | |
| | subunit D2 | | | | | | | | | |
| 1423290_PM_at | hypoxia up- | Hyou1 | 12282 | | | | | | 1.82 | 10525 |
| | regulated 1 | | | | | | | | | |
| 1433833_PM_at | fibronectin | Fndc3b | 72007 | | | | | | 1.66 | 64778 |
| | type III | | | | | | | | | |
| | domain | | | | | | | | | |
| | containing 3B | | | | | | | | | |
| 1416235_PM_at | leucine rich | Lrrc59 | 98238 | | | | | | 1.65 | 55379 |
| | repeat | | | | | | | | | |
| | containing 59 | | | | | | | | | |
| 1448471_PM_a_at | cytotoxic T | Ctla2a | 13024 | | | | | | 1.63 | NA |
| | lymphocyte- | | | | | | | | | |
| | associated | | | | | | | | | |
| | protein 2 | | | | | | | | | |
| | alpha | | | | | | | | | |
| 1448574_PM_at | non- | Nme6 | 54369 | | | | | | 1.57 | 10201 |
| | metastatic | | | | | | | | | |
| | cells 6, | | | | | | | | | |
| | protein | | | | | | | | | |
| | expressed in | | | | | | | | | |
| | (nucleoside- | | | | | | | | | |
| | diphosphate | | | | | | | | | |
| | kinase) | | | | | | | | | |
| 1443807_PM_x_at | cyclin F | Ccnf | 12449 | | | | | | 1.57 | 899 |
| 1450971_PM_at | growth arrest | Gadd45b | 17873 | | | | | | 1.50 | 4616 |
| | and DNA- | | | | | | | | | |
| | damage- | | | | | | | | | |
| | inducible 45 | | | | | | | | | |
| | beta | | | | | | | | | |
| 1453103_PM_at | actin-binding | Ablim1 | 226251 | | | | | | −1.51 | 3983 |
| | LIM protein 1 | | | | | | | | | |
| 1439117_PM_at | calmin | Clmn | 94040 | | | | | | −1.54 | 79789 |
| 1441988_PM_at | protein | Ppm1k | 243382 | | | | | | −1.56 | 152926 |
| | phosphatase | | | | | | | | | |
| | 1K (PP2C | | | | | | | | | |
| | domain | | | | | | | | | |
| | containing) | | | | | | | | | |
| 1460256_PM_at | carbonic | Car3 | 12350 | | | | | | −1.57 | 761 |
| | anhydrase 3 | | | | | | | | | |

TABLE 8
Murine Spleen DEGs

| | | | | | | | | | | |
|---|---|---|---|---|---|---|---|---|---|---|
| | | | | | | | | | | Human |
| | | | | DMF | DMF | DMF | MMF | MMF | MMF | Homolog |
| Probe Set | Gene Title | Gene | ENTREZ ID | 2 hr | 7 hr | 12 hr | 2 hr | 7 hr | 12 hr | Entrez ID |

| | | | | | | | | |
|---|---|---|---|---|---|---|---|---|
| 1419942_PM_at | Sulfiredoxin 1 | Srxn1 | 76650 | 2.16 | 1.67 | 2.02 | 1.87 | 140809 |
| | homolog | | | | | | | |
| | (S. cerevisiae) | | | | | | | |
| 1419435_PM_at | aldehyde | Aox1 | 11761 | 2.61 | | 2.35 | | 316 |
| | oxidase 1 | | | | | | | |
| 1434797_PM_at | kin of IRRE | Kirrel | 170643 | −1.54 | | −1.65 | | 55243 |
| | like | | | | | | | |
| | (Drosophila) | | | | | | | |
| 1424022_PM_at | oxidative stress | Osgin1 | 71839 | 1.78 | | 1.83 | | 29948 |
| | induced growth | | | | | | | |
| | inhibitor 1 | | | | | | | |
| 1416497_PM_at | protein | Pdia4 | 12304 | −1.60 | | −1.59 | | 9601 |
| | disulfide | | | | | | | |
| | isomerase | | | | | | | |
| | associated 4 | | | | | | | |
| 1424486_PM_a_at | thioredoxin | Txnrd1 | 50493 | 2.41 | | 2.14 | | 7296 |
| | reductase 1 | | | | | | | |
| 1424296_PM_at | glutamate- | Gclc | 14629 | 1.76 | | | | 2729 |
| | cysteine ligase, | | | | | | | |
| | catalytic | | | | | | | |
| | subunit | | | | | | | |
| 1448916_PM_at | v-maf | Mafg | 17134 | 1.58 | | | | 4097 |
| | musculoaponeurotic | | | | | | | |
| | fibrosarcoma | | | | | | | |
| | oncogene | | | | | | | |
| | family, protein | | | | | | | |
| | G (avian) | | | | | | | |
| 1434951_PM_at | armadillo repeat | Armc8 | 74125 | | | −1.54 | | 25852 |
| | containing 8 | | | | | | | |
| 1425521_PM_at | polyadenylate | Paip1 | 218693 | | | −1.56 | | 10605 |
| | binding protein- | | | | | | | |
| | interacting | | | | | | | |
| | protein 1 | | | | | | | |
| 1424084_PM_at | ROD1 regulator of | Rod1 | 230257 | | | −1.54 | | 9991 |
| | differentiation 1 | | | | | | | |
| | (S. pombe) | | | | | | | |

TABLE 9
Murine Transcript and protein level change in DEGs from blood.

| | | | | | DMF 12 hr MFI |
|---|---|---|---|---|---|
| | | | | | difference (compared |
| | | | Human | DMF 12 hr FC | to MMF 12 hr or |
| | Murine | Murine | Homolog | (transcriptional | vehicle control) (protein |
| Gene Title | Gene | Entrez ID | Entrez ID | fold change) | expression change) |
| Granzyme A | Gzma | 14938 | 3001 | 1.92 | Not tested |
| Natural | Ncr1 | 17086 | 9437 | 1.86 | Not significant |
| cytotoxicity | | | | | (ns; |
| triggering | | | | | p = 0.02) |
| receptor 1 | | | | | |
| Killer cell | Klrc1 | 16641 | 3821 | 1.85 | Significant |
| lectin-like | | | | | (p < 0.0001; |
| receptor | | | | | p = 0.003) |
| subfamily C, | | | | | |
| member 1 | | | | | |
| Killer cell | Klrb1b | 80782 | 3820 | 1.65 | Significant |
| lectin-like | | | | | (p = 0.0006; |
| receptor | | | | | p < 0.0001) |
| subfamily B, | | | | | |
| member 1B | | | | | |
| Killer cell | Klre1 | 243655 | | 1.85 | Not tested |
| lectin-like | | | | | |
| receptor | | | | | |
| family E, | | | | | |
| member 1 | | | | | |
| NKG2d | Klrk1 | 27007 | 22914 | n/a | Significant |
| | | | | | (p = 0.014; |
| | | | | | p = 0.0009) |
| Natural killer | Klrd1 | 16643 | 3824 | n/a | Significant |
| cells CD94 | | | | | (p = 0.0005; |
| | | | | | p = 0.014) |

### Probes and Methods for Detection

Probes and Methods for Detection of Translation Products

Probe-based methods, include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF) mass spectrometry, microcytometry, microarray, microscopy, fluorescence activated cell sorting (FACS), flow cytometry, laser scanning cytometry, hematology analyzer and assays based on a property of the protein including but not limited to DNA binding, ligand binding, or interaction with other protein partners.

The translation product or polypeptide can be detected and quantified by any of a number of means well known to those of skill in the art. These can include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, immunohistochemistry and the like. A skilled artisan can readily adapt known protein/antibody detection methods for use in determining the expression level of one or more biomarkers in a serum sample.

A useful probe for detecting a polypeptide is an antibody capable of binding to the polypeptide, e.g., an antibody with a detectable label. Antibodies can be polyclonal or monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

An antibody probe can be labeled, e.g., a radio-labeled, chromophore-labeled, fluorophore-labeled, or enzyme-labeled antibody. In another embodiment, an antibody derivative (e.g., an antibody conjugated with a substrate or with the protein or ligand of a protein-ligand pair {e.g., biotin-streptavidin}), or an antibody fragment (e.g., a single-chain antibody, an isolated antibody hypervariable domain, etc.) which binds specifically with a protein corresponding to the marker, such as the protein encoded by the open reading frame corresponding to the marker or such a protein which has undergone all or a portion of its normal post-translational modification, is used.

Immunohistochemistry or IHC refers to the process of localizing antigens (e.g. proteins), e.g., in cells of a tissue section or other sample, exploiting the principle of antibodies binding specifically to antigens in biological tissues. Specific molecular markers are characteristic of particular cellular events such as proliferation or cell death (apoptosis). Visualizing an antibody-antigen interaction can be accomplished in a number of ways. In the most common instance, an antibody is conjugated to an enzyme, such as peroxidase, that can catalyze a color-producing reaction. Alternatively, the antibody can also be tagged to a fluorophore, such as fluorescein, rhodamine, DyLight Fluor or Alexa Fluor.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

In one format, antibodies, or antibody fragments, can be used as probes in methods such as Western blots or immunofluorescence techniques to detect the expressed proteins. In such uses, one can immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another embodiment, Western blot (immunoblot) analysis is used to detect and quantify the presence of a polypeptide in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind a polypeptide. The anti-polypeptide antibodies specifically bind to the polypeptide on the solid support. These antibodies can be directly labeled or alternatively can be subsequently detected using labeled antibodies (e.g., labeled sheep anti-human antibodies) that specifically bind to the anti-polypeptide.

In another embodiment, the polypeptide is detected using an immunoassay. As used herein, an immunoassay is an assay that utilizes an antibody to specifically bind to the analyte. The immunoassay is thus characterized by detection of specific binding of a polypeptide to an anti-antibody as opposed to the use of other physical or chemical properties to isolate, target, and quantify the analyte.

The polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

In another embodiment, the polypeptide is detected and/or quantified using Luminex™ assay technology. The Luminex™ assay separates tiny color-coded beads into e.g., distinct sets that are each coated with a reagent for a particular bioassay, allowing the capture and detection of specific analytes from a sample in a multiplex manner. The Luminex™ assay technology can be compared to a multiplex ELISA assay using bead-based fluorescence cytometry to detect analytes such as biomarkers.

Immunological binding assays (or immunoassays) typically utilize a “capture agent” to specifically bind to and often immobilize the analyte (polypeptide or subsequence). The capture agent is a moiety that specifically binds to the analyte. In another embodiment, the capture agent is an antibody that specifically binds a polypeptide. The antibody (anti-peptide) can be produced by any of a number of means well known to those of skill in the art.

Immunoassays also often utilize a labeling agent to specifically bind to and label the binding complex formed by the capture agent and the analyte. The labeling agent can itself be one of the moieties comprising the antibody/analyte complex. Thus, the labeling agent can be a labeled polypeptide or a labeled anti-antibody. Alternatively, the labeling agent can be a third moiety, such as another antibody, that specifically binds to the antibody/polypeptide complex.

In one embodiment, the labeling agent is a second human antibody bearing a label. Alternatively, the second antibody can lack a label, but it can, in turn, be bound by a labeled third antibody specific to antibodies of the species from which the second antibody is derived. The second can be modified with a detectable moiety, e.g., as biotin, to which a third labeled molecule can specifically bind, such as enzyme-labeled streptavidin.

Other proteins capable of specifically binding immunoglobulin constant regions, such as protein A or protein G can also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

As indicated above, immunoassays for the detection and/or quantification of a polypeptide can take a wide variety of formats well known to those of skill in the art.

Exemplary immunoassays for detecting a polypeptide can be competitive or noncompetitive. Noncompetitive immunoassays are assays in which the amount of captured analyte is directly measured. In one “sandwich” assay, for example, the capture agent (anti-peptide antibodies) can be bound directly to a solid substrate where they are immobilized. These immobilized antibodies then capture polypeptide present in the test sample. The polypeptide thus immobilized is then bound by a labeling agent, such as a second human antibody bearing a label.

In competitive assays, the amount of analyte (polypeptide) present in the sample is measured indirectly by measuring the amount of an added (exogenous) analyte (polypeptide) displaced (or competed away) from a capture agent (anti-peptide antibody) by the analyte present in the sample. In one competitive assay, a known amount of, in this case, a polypeptide is added to the sample and the sample is then contacted with a capture agent. The amount of polypeptide bound to the antibody is inversely proportional to the concentration of polypeptide present in the sample.

In another embodiment, the antibody is immobilized on a solid substrate. The amount of polypeptide bound to the antibody can be determined either by measuring the amount of polypeptide present in a polypeptide/antibody complex, or alternatively by measuring the amount of remaining uncomplexed polypeptide. The amount of polypeptide can be detected by providing a labeled polypeptide.

The assays described herein are scored (as positive or negative or quantity of polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of polypeptide.

In another embodiment, level (activity) is assayed by measuring the enzymatic activity of the gene product. Methods of assaying the activity of an enzyme are well known to those of skill in the art.

In vivo techniques for detection of a marker protein include introducing into a subject a labeled antibody directed against the protein. For example, the antibody can be labeled with a radioactive marker whose presence and location in a subject can be detected by standard imaging techniques.

Certain markers identified by the methods of the invention can be secreted proteins. It is a simple matter for the skilled artisan to determine whether any particular marker protein is a secreted protein. In order to make this determination, the marker protein is expressed in, for example, a mammalian cell, e.g., a human cell line, extracellular fluid is collected, and the presence or absence of the protein in the extracellular fluid is assessed (e.g., using a labeled antibody which binds specifically with the protein).

Antibodies can be used a probes for translation products. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention. A molecule which specifically binds to a given polypeptide is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. Probes can be polyclonal or monoclonal antibodies. The term “monoclonal antibody” or “monoclonal antibody composition”, as used herein, refers to a population of antibody molecules that contain only one species of an antigen binding site capable of immunoreacting with a particular epitope.

An antibody directed against a polypeptide can be used to isolate the polypeptide by standard techniques, such as affinity chromatography or immunoprecipitation. Moreover, such an antibody can be used to detect the marker (e.g., in a cellular lysate or cell supernatant) in order to evaluate the level and pattern of expression of the marker. The antibodies can also be used diagnostically to monitor protein levels in tissues or body fluids (e.g., in a tumor cell-containing body fluid) as part of a clinical testing procedure, e.g., to, for example, determine the efficacy of a given treatment regimen. Detection can be facilitated by coupling the antibody to a detectable substance. Examples of detectable substances include, but are not limited to, various enzymes, prosthetic groups, fluorescent materials, luminescent materials, bioluminescent materials, and radioactive materials. Examples of suitable enzymes include, but are not limited to, horseradish peroxidase, alkaline phosphatase, β-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin, and examples of suitable radioactive materials include, but are not limited to, ¹²⁵I, ¹³¹I, ³⁵S or ³H.

Probes and Methods for Detection of Transcription Products

Translational expression can be assessed by any of a wide variety of well known methods for detecting expression. Non-limiting examples of such methods include nucleic acid hybridization methods, nucleic acid reverse transcription methods, and nucleic acid amplification methods.

In certain embodiments, activity of a particular gene is characterized by a measure of gene transcript (e.g., mRNA). Detection can involve quantification of the level of gene expression (e.g., cDNA, mRNA), or, alternatively, can be a qualitative assessment of the level of gene expression, in particular in comparison with a control level. The type of level being detected will be clear from the context.

Methods of detecting and/or quantifying the gene transcript (mRNA or cDNA made therefrom) using nucleic acid hybridization techniques are known to those of skill in the art (see e.g., Sambrook et al. supra). For example, one method for evaluating the presence, absence, or quantity of cDNA involves a Southern transfer as described above. Briefly, the mRNA is isolated (e.g., using an acid guanidinium-phenol-chloroform extraction method, Sambrook et al. supra.) and reverse transcribed to produce cDNA. The cDNA is then optionally digested and run on a gel in buffer and transferred to membranes. Hybridization is then carried out using the nucleic acid probes specific for the target cDNA.

A general principle of such diagnostic and prognostic assays involves preparing a sample or reaction mixture that can contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, polyethylene, dextran, amylases, natural and modified celluloses, polyacrylamides, gabbros, and magnetite.

In order to conduct assays with the above-mentioned approaches, the non-immobilized component is added to the solid phase upon which the second component is anchored. After the reaction is complete, uncomplexed components can be removed (e.g., by washing) under conditions such that any complexes formed will remain immobilized upon the solid phase. The detection of marker/probe complexes anchored to the solid phase can be accomplished in a number of methods outlined herein.

In another embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes can be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques can also be utilized to separate complexed molecules from uncomplexed ones. For example, gel filtration chromatography separates molecules based on size, and through the utilization of an appropriate gel filtration resin in a column format, for example, the relatively larger complex can be separated from the relatively smaller uncomplexed components. Similarly, the relatively different charge properties of the marker/probe complex as compared to the uncomplexed components can be exploited to differentiate the complex from uncomplexed components, for example, through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis can also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typical. Appropriate conditions to the particular assay and components thereof will be well known to one skilled in the art.

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated nucleic acid can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

The probes can be full length or less than the full length of the nucleic acid sequence encoding the protein. Shorter probes are empirically tested for specificity. Exemplary nucleic acid probes are 20 bases or longer in length (See, e.g., Sambrook et al. for methods of selecting nucleic acid probe sequences for use in nucleic acid hybridization). Visualization of the hybridized portions allows the qualitative determination of the presence or absence of cDNA.

An alternative method for determining the level of a transcript involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. Fluorogenic rtPCR can also be used in the methods of the invention. In fluorogenic rtPCR, quantitation is based on amount of fluorescence signals, e.g., TaqMan and sybr green. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations can be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a healthy subject, or between samples from different sources.

Alternatively, the expression level can be provided as a relative expression level. To determine a relative expression level of a marker, the level of expression of the marker is determined for 10 or more samples of normal versus MS isolates, or even 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

In certain embodiments, the samples used in the baseline determination will be from samples derived from a subject having multiple sclerosis versus samples from a healthy subject of the same tissue type. The choice of the cell source is dependent on the use of the relative expression level. Using expression found in normal tissues as a mean expression score aids in validating whether the marker assayed is specific to the tissue from which the cell was derived (versus normal cells). In addition, as more data is accumulated, the mean expression value can be revised, providing improved relative expression values based on accumulated data. Expression data from normal cells provides a means for grading the severity of the multiple sclerosis disease state.

In another embodiment, expression of a marker is assessed by preparing mRNA/cDNA (i.e., a transcribed polynucleotide) from cells in a subject sample, and by hybridizing the genomic DNA or mRNA/cDNA with a reference polynucleotide which is a complement of a polynucleotide comprising the marker, and fragments thereof. cDNA can, optionally, be amplified using any of a variety of polymerase chain reaction methods prior to hybridization with the reference polynucleotide. Expression of one or more markers can likewise be detected using quantitative PCR (QPCR) to assess the level of expression of the marker(s). Alternatively, any of the many known methods of detecting mutations or variants (e.g., single nucleotide polymorphisms, deletions, etc.) of a marker of the invention can be used to detect occurrence of a mutated marker in a subject.

In a related embodiment, a mixture of transcribed polynucleotides obtained from the sample is contacted with a substrate having fixed thereto a polynucleotide complementary to or homologous with at least a portion (e.g., at least 7, at least 10, at least 15, at least 20, at least 25, at least 30, at least 40, at least 50, at least 100, at least 500, or more nucleotide residues) of a marker of the invention. If polynucleotides complementary to or homologous with a marker of the invention are differentially detectable on the substrate (e.g., detectable using different chromophores or fluorophores, or fixed to different selected positions), then the levels of expression of a plurality of markers can be assessed simultaneously using a single substrate (e.g., a “gene chip” microarray of polynucleotides fixed at selected positions). When a method of assessing marker expression is used which involves hybridization of one nucleic acid with another, the hybridization can be performed under stringent hybridization conditions.

In another embodiment, a combination of methods to assess the expression of a marker is utilized.

Because the compositions, kits, and methods of the invention rely on detection of a difference in expression levels of one or more markers of the invention, in certain embodiments the level of expression of the marker is significantly greater than the minimum detection limit of the method used to assess expression in at least one of a biological sample from a subject with MS or a healthy control.

A nucleic acid molecule of the invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

Probes based on the sequence of a nucleic acid molecule of the invention can be used to detect transcripts (e.g., mRNA) or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The methods described herein can also include molecular beacon nucleic acid molecules having at least one region which is complementary to a nucleic acid molecule of the invention, such that the molecular beacon is useful for quantitating the presence of the nucleic acid molecule of the invention in a sample. A “molecular beacon” nucleic acid is a nucleic acid molecule comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid molecules are not annealed with one another, fluorescence of the fluorophore is quenched to a lesser degree. Molecular beacon nucleic acid molecules are described, for example, in U.S. Pat. No. 5,876,930.

### Kits

A kit is any manufacture (e.g., a package or container) comprising at least one reagent, e.g., a probe, e.g., a nucleic acid probe or an antibody, for specifically detecting a translation or transcription product described herein.

The invention also encompasses kits having probes for detecting the presence of a polypeptide or nucleic acid in a biological sample, e.g., a sample containing tissue, whole blood, serum, plasma, buccal scrape, saliva, cerebrospinal fluid, urine, stool, and bone marrow. For example, the kit can comprise a labeled compound or agent capable of detecting a polypeptide or an mRNA encoding a polypeptide in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

A kit can include a plurality of probes for detecting a plurality of translation or transcription products. If a plurality of expression products are to be analysed the kit can comprise a probe for each.

The kit can comprise one or more probes capable of identifying one or more of gene products described herein, e.g., gene products identified herein (e.g., the markers set forth in Table 9). Suitable probes for a polypeptide include antibodies, antibody derivatives, antibody fragments, and the like. Suitable probes for a transcription product include a nucleic acid, e.g., complementary nucleic acids. For example, a kit can include oligonucleotides (labeled or non-labeled) fixed to a substrate, labeled oligonucleotides not bound with a substrate, pairs of PCR primers, molecular beacon probes, and the like.

The kit of the invention can optionally comprise additional components useful for performing the methods of the invention. By way of example, the kit can comprise fluids (e.g., SSC buffer) suitable for annealing complementary nucleic acids or for binding an antibody with a protein with which it specifically binds, one or more sample compartments, an instructional material which describes performance of a method of the invention, a reference sample for comparison of expression levels of the biomarkers described herein, and the like.

A kit can include a device described herein.

For antibody-based kits, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

regions.

### MS Therapeutic Agents, Compositions and Administration

There are several medications presently used to modify the course of multiple sclerosis. Such agents include, but are not limited to, dialkyl fumarates (e.g., DMF or others of Formula A herein), Beta interferons (e.g., Avonex®, Rebif®, Betaseron®, Betaferon®, among others)), glatiramer (Copaxone®), natalizumab (Tysabri®), and mitoxantrone (Novantrone®).

“Treat,” “treatment,” and other forms of this word refer to the administration of an agent, e.g., an agent described herein, alone or in combination with one or more symptom management agents, to a subject, e.g., an MS patient, to impede progression of multiple sclerosis, to induce remission, to extend the expected survival time of the subject and or reduce the need for medical interventions (e.g., hospitalizations). In those subjects, treatment can include, but is not limited to, inhibiting or reducing one or more symptoms such as numbness, tingling, muscle weakness; reducing relapse rate, reducing size or number of sclerotic lesions; inhibiting or retarding the development of new lesions; prolonging survival, or prolonging progression-free survival, and/or enhanced quality of life.

As used herein, unless otherwise specified, the terms “prevent,” “preventing” and “prevention” contemplate an action that occurs before a subject begins to suffer from the a multiple sclerosis relapse and/or which inhibits or reduces the severity of the disease.

As used herein, and unless otherwise specified, the terms “manage,” “managing” and “management” encompass preventing the progression of MS symptoms in a patient who has already suffered from the disease, and/or lengthening the time that a patient who has suffered from MS remains in remission. The terms encompass modulating the threshold, development and/or duration of MS, or changing the way that a patient responds to the disease.

As used herein, and unless otherwise specified, a “therapeutically effective amount” of a compound is an amount sufficient to provide a therapeutic benefit in the treatment or management of multiple sclerosis, or to delay or minimize one or more symptoms associated with MS. A therapeutically effective amount of a compound means an amount of therapeutic agent, alone or in combination with other therapeutic agents, which provides a therapeutic benefit in the treatment or management of MS. The term “therapeutically effective amount” can encompass an amount that improves overall therapy, reduces or avoids symptoms or causes of the disease, or enhances the therapeutic efficacy of another therapeutic agent.

As used herein, and unless otherwise specified, a “prophylactically effective amount” of a compound is an amount sufficient to prevent relapse of MS, or one or more symptoms associated with the disease, or prevent its recurrence. A prophylactically effective amount of a compound means an amount of the compound, alone or in combination with other therapeutic agents, which provides a prophylactic benefit in the prevention of MS relapse. The term “prophylactically effective amount” can encompass an amount that improves overall prophylaxis or enhances the prophylactic efficacy of another prophylactic agent.

As used herein, the term “patient” or “subject” refers to an animal, typically a human (i.e., a male or female of any age group, e.g., a pediatric patient (e.g., infant, child, adolescent) or adult patient (e.g., young adult, middle-aged adult or senior adult) or other mammal, such as a primate (e.g., cynomolgus monkey, rhesus monkey); commercially relevant mammals such as cattle, pigs, horses, sheep, goats, cats, and/or dogs; and/or birds, including commercially relevant birds such as chickens, ducks, geese, and/or turkeys, that will be or has been the object of treatment, observation, and/or experiment. When the term is used in conjunction with administration of a compound or drug, then the patient has been the object of treatment, observation, and/or administration of the compound or drug.

The methods described herein permit one of skill in the art to identify a monotherapy that an MS patient is most likely to respond to, thus eliminating the need for administration of multiple therapies to the patient to ensure that a therapeutic effect is observed. However, in one embodiment, combination treatment of an individual with MS is contemplated.

It will be appreciated that the MS therapies, as described above and herein, can be administered in combination with one or more additional therapies to treat and/or reduce the symptoms of MS described herein, particularly to treat patients with moderate to severe disability (e.g., EDSS score of 5.5 or higher). The pharmaceutical compositions can be administered concurrently with, prior to, or subsequent to, one or more other additional therapies or therapeutic agents. In general, each agent will be administered at a dose and/or on a time schedule determined for that agent. In will further be appreciated that the additional therapeutic agent utilized in this combination can be administered together in a single composition or administered separately in different compositions. The particular combination to employ in a regimen will take into account compatibility of the pharmaceutical composition with the additional therapeutically active agent and/or the desired therapeutic effect to be achieved. In general, it is expected that additional therapeutic agents utilized in combination be utilized at levels that do not exceed the levels at which they are utilized individually. In some embodiments, the levels utilized in combination will be lower than those utilized individually.

### Alternative or Other Therapies

In other embodiments, alternative therapies to the DMF can be administered.

In one embodiment, the alternative therapy includes an interferon beta, a polymer of four amino acids found in myelin basic protein, e.g., a polymer of glutamic acid, lysine, alanine and tyrosine (e.g., glatiramer (Copaxone®)). In other embodiments, the alternative therapy includes an antibody or fragment thereof against alpha-4 integrin (e.g., natalizumab (Tysabri®)). In yet other embodiments, the alternative therapy includes an anthracenedione molecule (e.g., mitoxantrone (Novantrone®)). In yet another embodiment, the alternative therapy includes a fingolimod (e.g., FTY720; Gilenya®). In other embodiments, the alternative therapy is an antibody to the alpha subunit of the IL-2 receptor of T cells (e.g., Daclizumab; described in, e.g., Rose, J. W. et al. (2007) Neurology 69 (8): 785-789). In yet other embodiments, the alternative therapy is an antibody against CD52 (e.g., alemtuzumab (Lemtrada®)). In yet another embodiment, the alternative therapy includes an anti-LINGO-1 antibody (described in, e.g., U.S. Pat. No. 8,058,406, entitled “Composition comprising antibodies to LINGO or fragments thereof.”).

Steroids, e.g., corticosteroid, and ACTH agents can be used to treat acute relapses in relapsing-remitting MS or secondary progressive MS. Such agents include, but are not limited to, Depo-Medrol®, Solu-Medrol®, Deltasone®, Delta-Cortef®, Medrol®, Decadron®, and Acthar®.

### Treatment of Other Disorders

Dialkyl fumarates, e.g., those of Formula A, can be used to treat NK function related disorders and conditions. While not wishing to be bound by theory it is believed that these disorders are ameliorated by NK cells. Such disorders include: cancer, e.g., hematopoietic malignancies, e.g., acute lymphocytic leukemia, chronic lymphocytic leukemia, and lymphoma; solid tumors, e.g., gastrointestinal sarcoma, neuroblastoma, and kidney cancer; viral infection; autoimmune disorders; and inflammation. Such conditions also include transplantation, e.g., solid organ transplantation, and GVHD.

This invention is further illustrated by the following examples which should not be construed as limiting. The contents of all references, figures, sequence listing, patents and published patent applications cited throughout this application are hereby incorporated by reference.

### EXAMPLES

### Example 1

### Transcriptional Profiling of Pharmacodynamic Effects of DMF and MMF

Tecfidera (BG-12, dimethyl fumarate, DMF) is an oral therapeutic approved in the U.S., Canada and Australia for the treatment of relapsing multiple sclerosis (MS). The mechanism by which Tecfidera exerts clinical effects is unknown, but preclinical studies indicate activation of the nuclear factor (erythroid-derived 2)-like 2(Nrf2) pathway is involved. Preclinical studies indicate that DMF promotes anti-inflammatory and neuroprotective responses, both of which may be useful in amelioration of MS pathophysioliogy. In vivo, DMF is rapidly metabolized to monomethyl fumarate (MMF), and both compounds are pharmacologically active.

In vitro, DMF and MMF share some common effects, but also have divergent pharmacological properties. To understand if in vitro differences translate into differential in vivo biology, DMF and MMF pharmacodynamic responses were characterized and compared in mice. This example describes the discovery and evaluation of differential transcriptional responses in multiple tissues and whole blood after oral dosing of DMF or MMF.

C57BL/6 mice were dosed with vehicle, DMF or MMF (100 mg/kg) and sacrificed at 2, 7, and 12 hours. Tissues (liver, spleen, kidney, jejunum, cortex, hippocampus, striatum and whole blood) were collected and analyzed by transcriptional profiling on mouse Affymetrix GeneChips. Differentially expressed genes were identified by comparing DMF or MMF treated mice to matched vehicle controls.

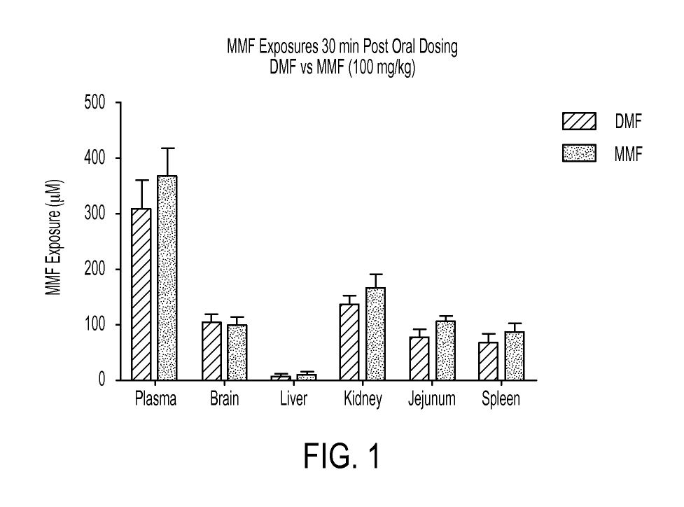

A separate cohort was sacrificed 30 minutes after dosing to evaluate MMF exposure. More specifically, satellite 5 animals per group were dosed orally with DMF or MMF (100 mg/kg) and sacrificed 30 minutes post-dosing. MMF exposures were determined and compared in various compartments. These analyses demonstrate that in mice receiving DMF or MMF, no significant differences in MMF exposure was observed, and this was consistent across tissues. As shown in FIG. 1, MMF levels were highly comparable between DMF and MMF dosing, suggesting any subsequent differences in pharmacodynamic responses were not simply due to different exposures.

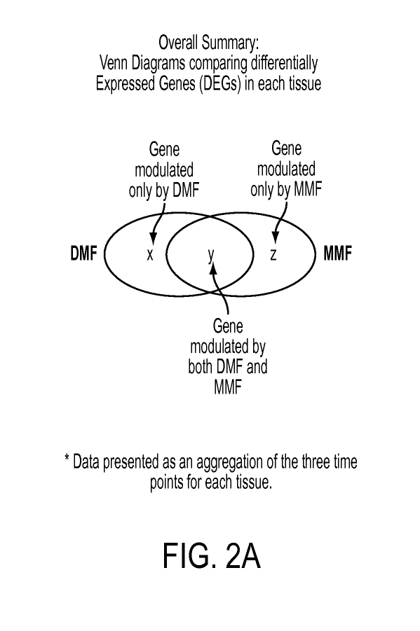

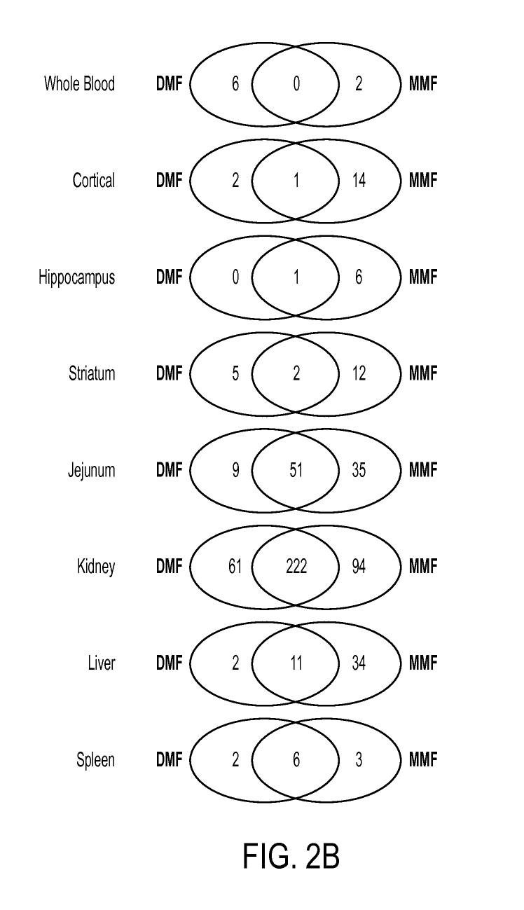

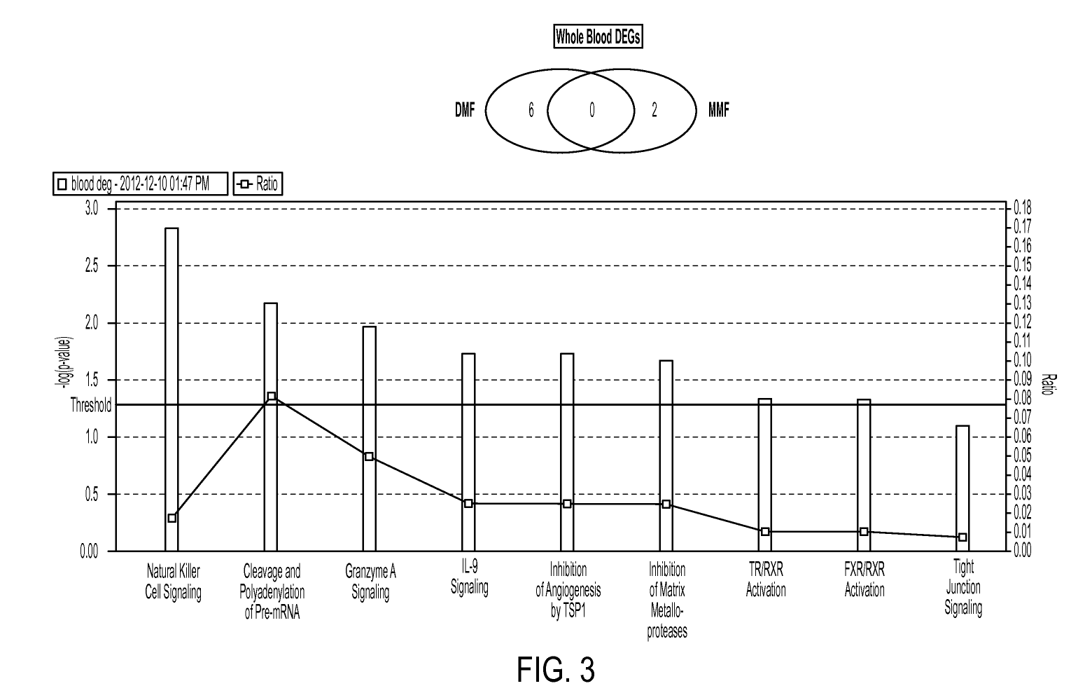

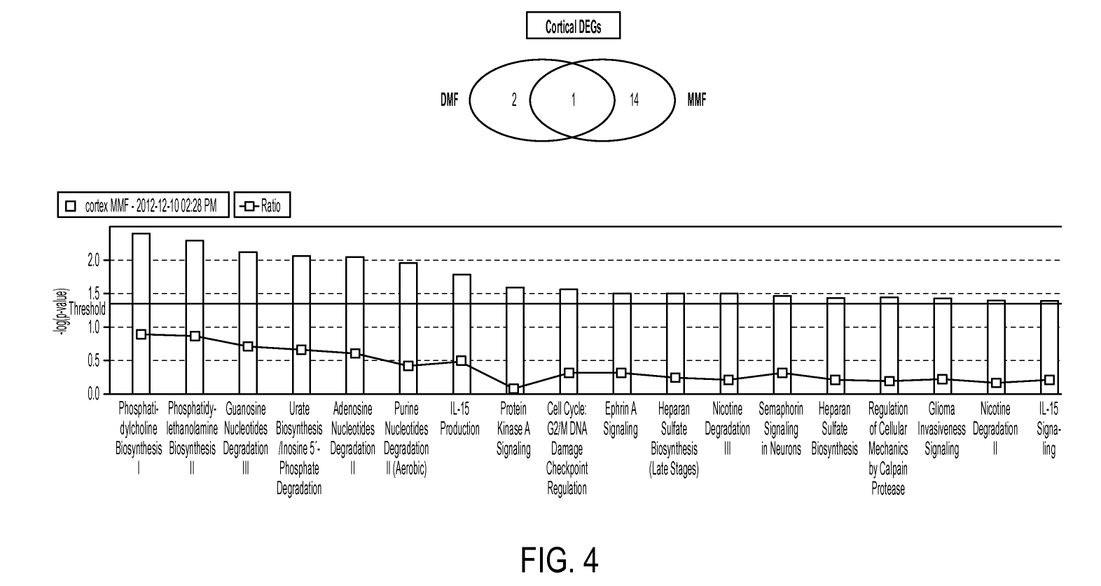

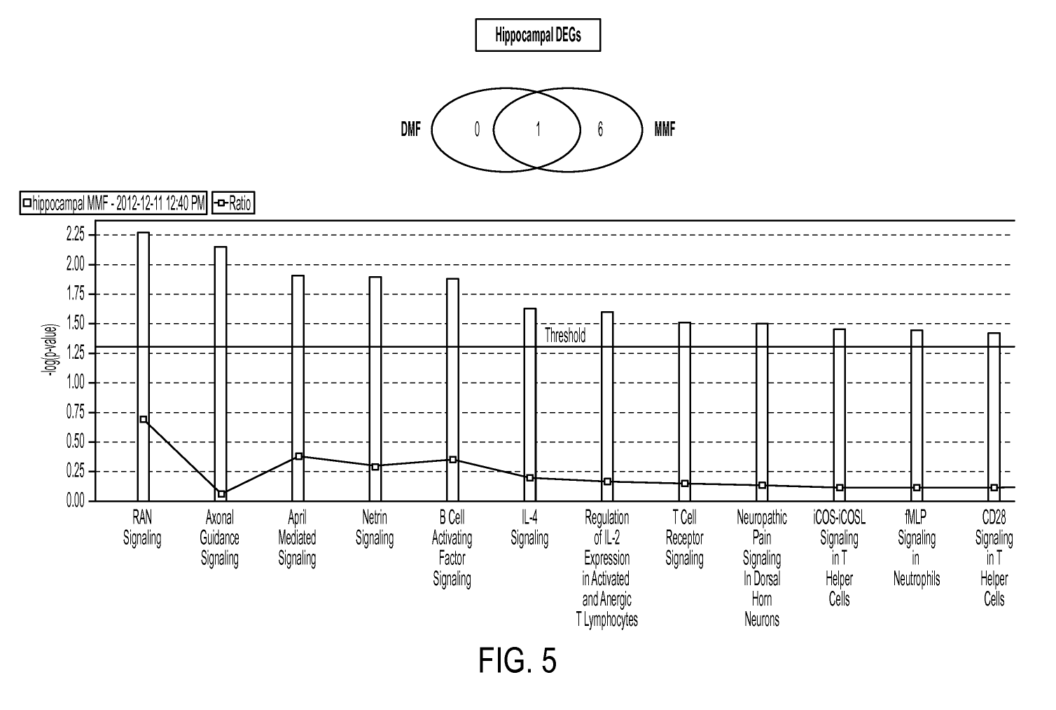

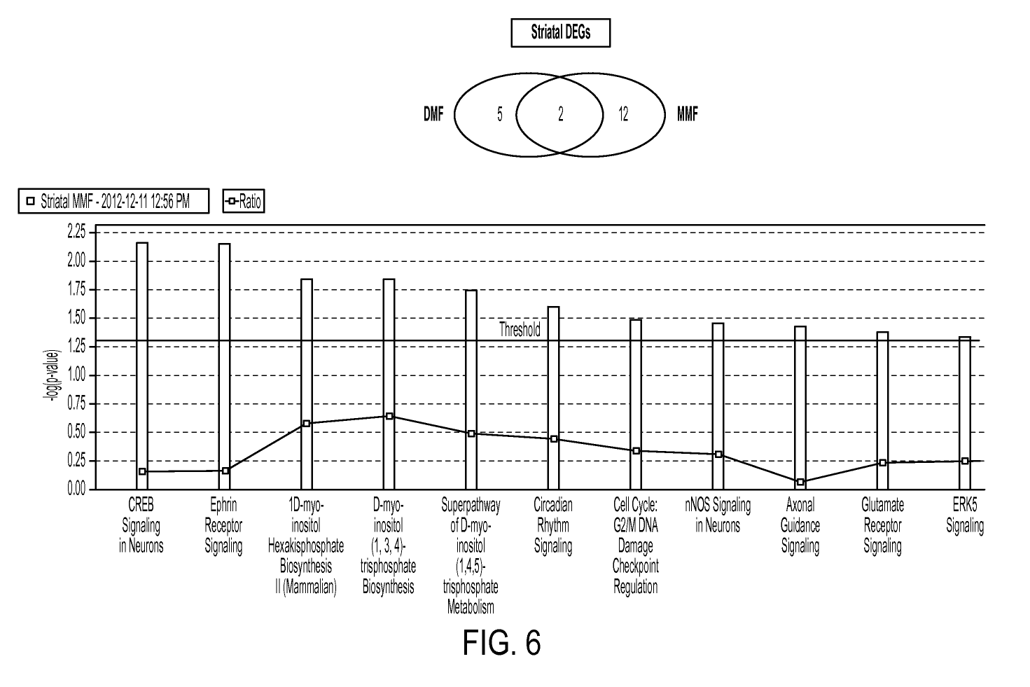

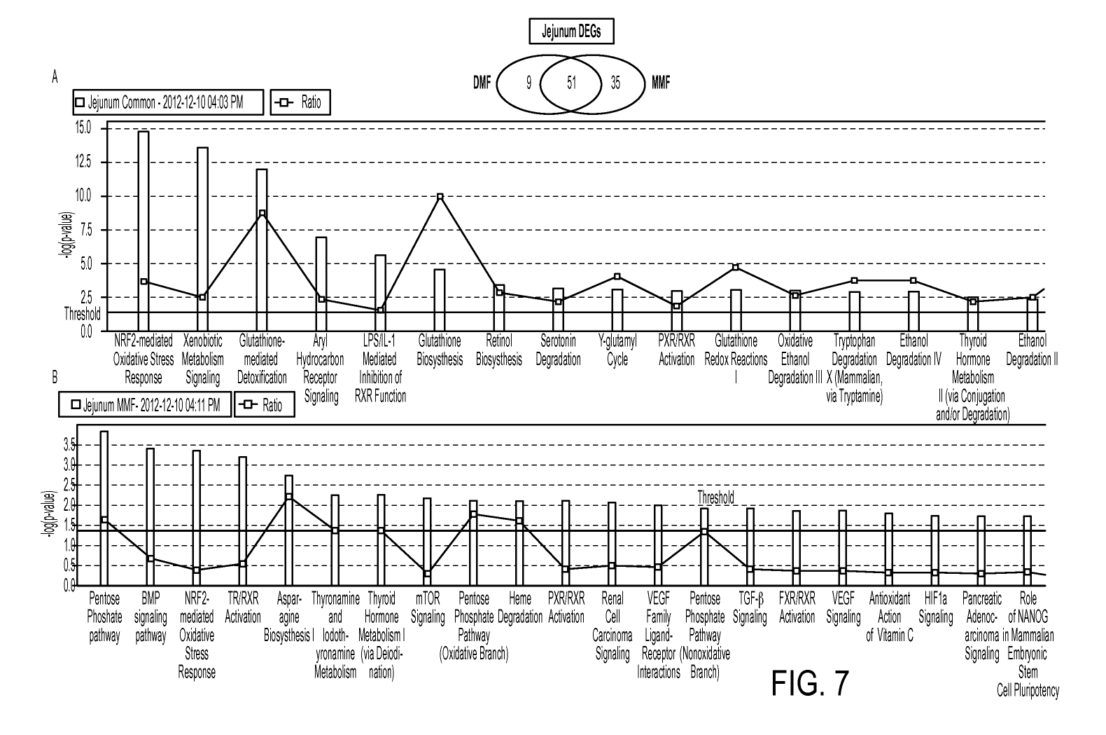

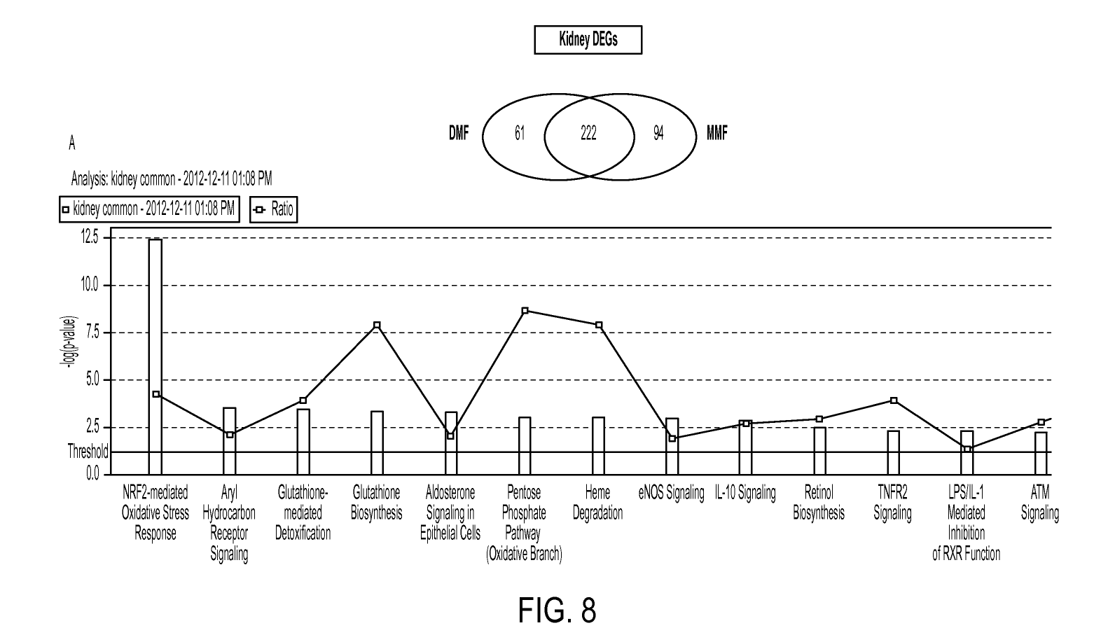

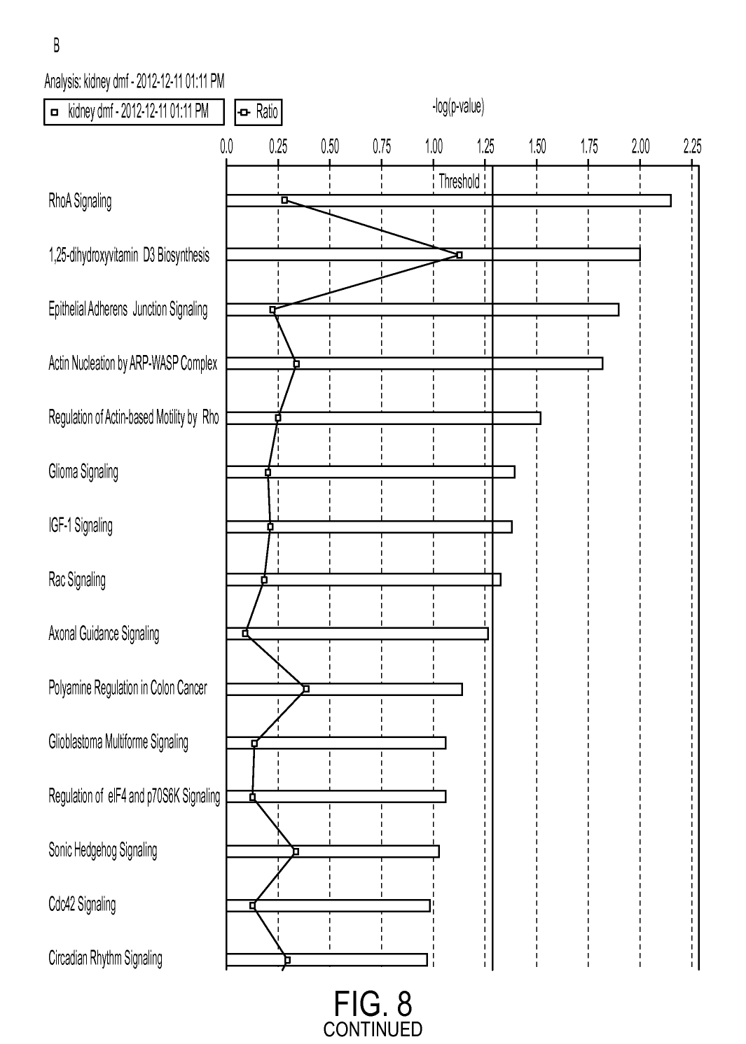

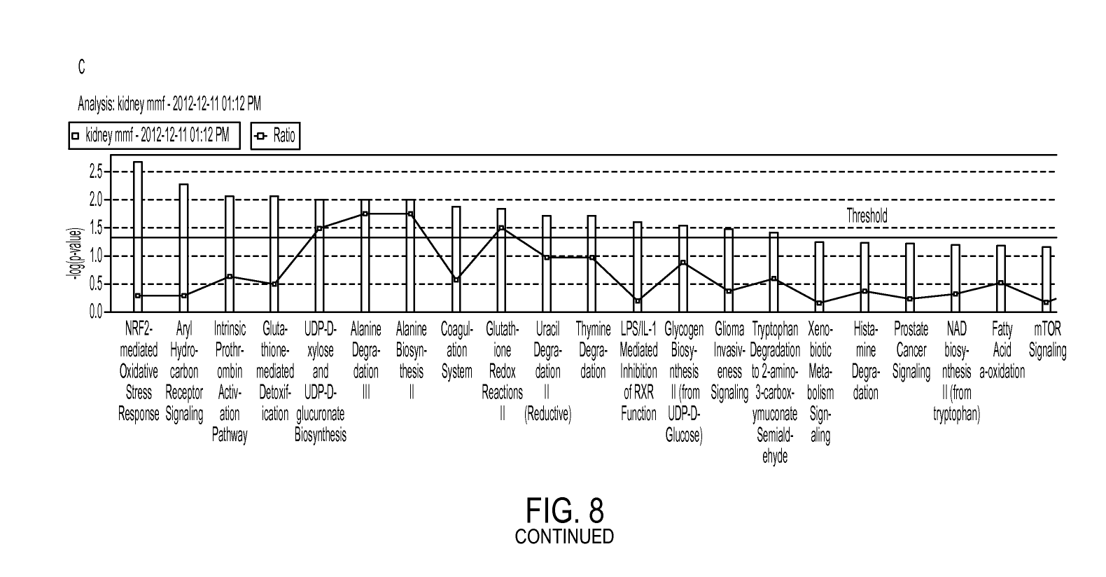

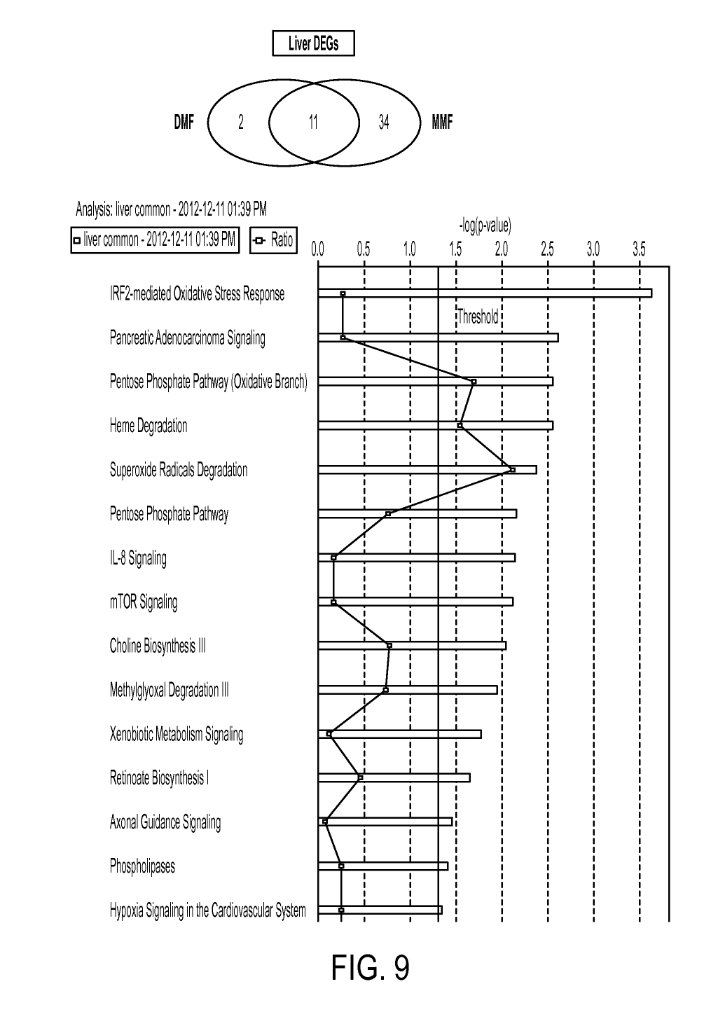

A specific transcriptional response to DMF and MMF treatment was observed in all tissues and whole blood. The overall number and identity of differentially regulated transcripts varied between tissues and treatment (FIG. 2). In most tissues, a similar trend was observed comparing genes regulated by DMF to those by MMF; a common set of transcripts induced by both treatments was identified, with a strong association with Nrf2 pathway activation. Additionally, treatment-specific transcripts were also identified. For example, differentially expressed genes (DEGs) from liver, spleen, kidney, jejunum, cortex, hippocampus, striatum and whole blood are shown in TABLES 1-8, above. Furthermore, DEGs identified from these tissues were analyzed to identify various pathways and cell types that may be involved in DMF and MMF pharmacological activity (FIGS. 3-9). Transcriptional differences were observed, for example, in NK cell markers identified in blood (FIG. 3), including Granzyme A (Gzma), natural cytotoxicity triggering receptor 1 (Ncr1), killer cell lectin-like receptor subfamily C, member 1 (Klrc1), killer cell lectin-like receptor subfamily B, member 1B (Klrb1b), and killer cell lectin-like receptor family E, member 1 (Klre1) (TABLE 1). These date demonstrate that several DMF differentially expressed genes identified in the transcriptional analysis effect NK cell function, and may indicate a link to macrophage signaling.

The incomplete overlap of transcriptional signatures induced by DMF and MMF indicates that not all DMF pharmacodynamic effects are conveyed through MMF, as may have been predicted due to the rapid in vivo metabolism of DMF to MMF. This suggests DMF may directly drive unique pharmacology not captured by MMF alone. Characterizing the potential biological consequences of these responses, such as effects on NK cells, and how they may contribute to the therapeutic benefit derived from oral administration of DMF, will be further investigated.

### Example 2

### Evaluation of NK Cell Surface Markers in Naïve Mice Receiving DMF or MMF

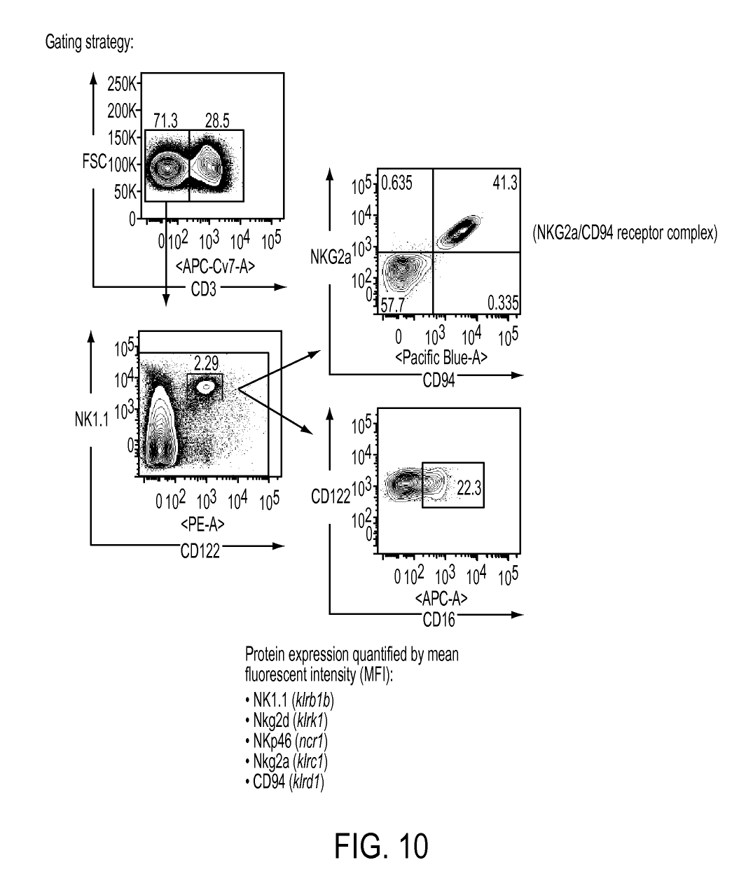

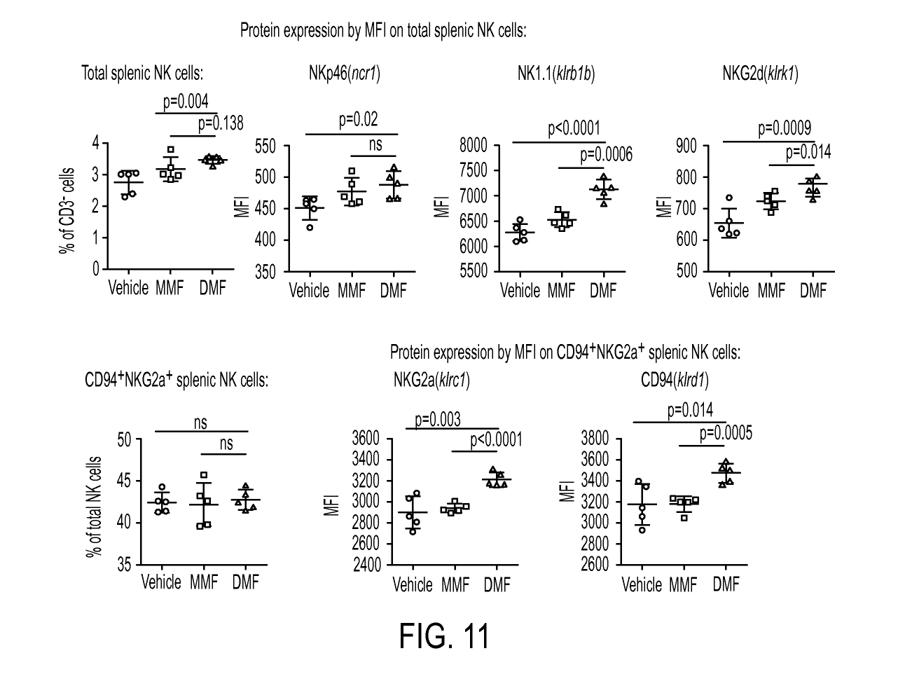

Transcriptional differences in NK cell markers were confirmed at the protein level by flow cytometry. FIG. 10 depicts an exemplary flow cytometry gating strategy for comparative analysis of DMF and MMF on Natural Killer (NK) cell phenotype. Protein expression was quantified by mean fluorescent intensity (MFI). A comparative analysis of DMF and MMF on NK cell phenotype was performed using the following markers: NK1.1 (klrb1b), Nkg2d (klrk1), NKp46 (ncr1), Nkg2a (klrc1), and CD94 (klrd1). Naïve C57Bl/6 mice were dosed PO with 100 mpk dimethyl fumarate (DMF) or molar equivalency of monomethyl fumarate (MMF). 12-hours post-dose, mice were sacrificed and blood, spleen, and inguinal lymph node (iLN) were collected for analysis by flow cytometry. FIG. 11 shows protein expression by MFI on total splenic NK cells (top) and on CD94+NKG2a+ splenic NK cells (bottom). TABLE 9 summarizes protein expression level differences between DMF 12 hour timepoint as compared to MMF 12 hour time point or vehicle-treated controls. DMF differentially expressed proteins as determined by MFI included Klrc1, Klrb1b, Klrk1 and Klrd1.

Among other things, these data confirm and expand the findings described in Example 1. For example, flow cytometry analysis confirmed transcriptional data identifying a number of DMF specific transcriptional changes related to NK cell function in blood. Furthermore, the data demonstrate that DMF exerts effects on NK cells in the spleen that were not observed with MMF.

### Example 3

### Transcriptional Profiling of Pharmacodynamic Effects of DMF and MMF in Naïve Mice

In vivo, DMF is rapidly metabolized to monomethyl fumarate (MMF). The field has long sought to define the relative contributions of DMF and MMF to the therapeutic benefit derived from BG-12. Although only MMF can be detected in systemic circulation following an oral dose of DMF, clinically and preclinically, DMF conjugates have been detected in urine indicating that some DMF survives first pass metabolism. Additionally, in patients receiving BG-12, there is likely exposure to DMF in the intestines once the enteric coated microtablets dissolve and the active pharmaceutical ingredient is released. Understanding the direct effects of DMF and MMF in vitro and in vivo would provide important insights into the mechanisms of action of BG-12. This example demonstrates transcriptional profiling of pharmacodynamic effects of oral administration of DMF and MMF in single or multi-dose regiments in naïve mice.

The number of independent mice whose tissues were harvested for transcript profiling studies and whose data passed QC is shown in TABLES 10 and 11.

TABLE 10
Number of replicates used in each treatment cohort for gene expression analyses.

| | | | Time | | | | | | | | | | |
|---|---|---|---|---|---|---|---|---|---|---|---|---|---|
| Animal | Dosing | Treat- | after last | | Whole | | | | | | | | |
| Model | Regimen | ment | dosing | | blood | Cerebellum | Cortex | Hippocampus | Striatum | Jenjumum | Kidney | Liver | Spleen |
| Naïve | Single | 100 mpk | 2 | h | 10 | 10 | 10 | 10 | 10 | 9 | 9 | 9 | 9 |
| | Dose | DMF | 7 | h | 10 | 10 | 10 | 10 | 10 | 10 | 10 | 10 | 10 |
| | | | 12 | h | 10 | 10 | 10 | 9 | 9 | 10 | 10 | 10 | 10 |
| | | 100 mpk | 2 | h | 10 | 10 | 9 | 10 | 10 | 9 | 10 | 10 | 8 |
| | | MMF | 7 | h | 9 | 10 | 10 | 10 | 10 | 10 | 9 | 10 | 10 |
| | | | 12 | h | 10 | 9 | 9 | 9 | 8 | 10 | 10 | 10 | 10 |
| | | Vehicle | 2 | h | 10 | 9 | 10 | 9 | 10 | 8 | 9 | 9 | 9 |
| | | | 7 | h | 10 | 10 | 10 | 10 | 10 | 10 | 10 | 10 | 10 |
| | | | 12 | h | 9 | 10 | 10 | 9 | 10 | 10 | 10 | 10 | 10 |

TABLE 11
Number of replicates used in each treatment cohort for gene expression analyses.

| | | | Time | | | | | | |
|---|---|---|---|---|---|---|---|---|---|
| Animal | Dosing | Treat- | after last | Whole | Brain | | Spinal | | Lymph |
| Model | Regimen | ment | dosing | blood | Hemi | Cerebellum | Cord | Spleen | Node |
| Naïve | Single | 100 mpk | 12 h | 10 | 9 | 8 | 9 | 9 | 10 |
| | Dose | DMF | | | | | | | |
| | | 100 mpk | | 9 | 8 | 10 | 10 | 10 | 9 |
| | | MMF | | | | | | | |
| | | Vehicle | | 10 | 9 | 10 | 9 | 9 | 10 |

### Materials and Methods

### Tissue Harvest

Animals were exposed to CO₂ and whole blood collected via cardiac puncture. Two 100 μl aliquots of whole blood were collected in 1.5 mL microcentrifuge tubes and snap frozen in liquid nitrogen. Peripheral (liver, spleen, kidney and jejunum) and CNS brain (cerebellum, hippocampus, striatum and cortex) tissues were harvested and snap frozen in liquid nitrogen, with special care taken to collect tissues of similar size and from the same location. All samples were stored at −80° C. until RNA extraction was conducted.

### Tissue RNA Extraction

For RNA preparation, frozen tissues were placed in 2 mL RNAse-free 96-well blocks with 1.5 mL QIAzol Lysis Reagent (QIAgen) and a 3.2 mm stainless steel bead (BioSpec Products, Bartlesville, Okla.). Tissues were disrupted for four cycles of 45 seconds in a Mini-Beadbeater (Biospec Products). RNA was extracted in chloroform and the aqueous phase was mixed with an equal volume of 70% ethanol. Extracted RNA was applied to RNeasy 96 plates and purified by the spin method according to the manufacturer's protocol (RNeasy 96 Universal Tissue Protocol, QIAgen, Hilden Germany).

Blood RNA Extraction and QC (Allaire N E, et al. BMC Res Notes. 2013 Jan. 5; 6:8)

50 ul of snap frozen mouse blood was re-suspended in lysis buffer with proteinase K and arrayed into deep-well plates for automated RNA extraction. RNA extractions were completed on Arrayplex (Beckman Coulter, Indianapolis, Ind.) using Agencourt RNAdvance Blood kit (Part number A35604) according to the manufacturer's specifications. RNA integrity was assessed using the HT RNA reagent kit (Part number 760410, Caliper Life Sciences, Hopkinton, Mass.) using a LabChip GX (PerkinElmer, Waltham, Mass.). RNA samples with a RQS score of >8.0 were considered high quality for downstream microarray processing.

### Sample Labeling, Hybridization and Scanning

Automated sample amplifications and biotin labelings were carried out using the NuGEN Ovation RNA Amplification system V2 (Cat #3100), Ovation WB reagent (Cat #1300) and Encore Biotin module (Cat #4200) (NuGEN Technologies, Inc, San Carlos, Calif.) according to manufacturer's recommendations using an Arrayplex automated liquid handler (Beckman Coulter, Indianapolis, Ind.). Two micrograms of biotin labeled sscDNA probe were hybridized to Affymetrix GeneChip Affymetrix HT_MG-430_PM plate arrays with modified conditions as described in Allaire et al. Washing and staining of the hybridized arrays were completed as described in the GeneChip Expression analysis technical manual for HT plate arrays using the Genechip® Array Station (Affymetrix, Santa Clara, Calif.) with modifications as described in Allaire et al. The processed GeneChip® plate arrays were scanned using GeneTitan scanner (Affymetrix, Santa Clara, Calif.).

### Analysis of Affymetrix Data

Affymetrix scans were subject to quality control (QC) measures. These tests included a visual inspection of the distribution of raw signal intensities and an assessment of RNA degradation, relative log expression (RLE), and normalized unsealed standard error (NUSE). All sample scans that passed these QC metrics were included in the analysis.

All CEL files were subjected to GC-content-based Robust Multi-array Average (GCRMA) normalization (version 2.20.0) (Irizarry R A, et al. Nucleic Acids Res 2003; 31:e15; Li C, et al. Genome Biol 2001; 2:RESEARCH0032). Expression levels were log (base 2) transformed.

Analyses were applied to discover genes that were differentially expressed (DEGs) between different animal treatments. The contrasts carried out are shown in TABLES 12 and 13. To identify differentially expressed genes between groups of samples, we applied the linear modeling approach (ANOVA) to fit gene expression levels (log 2 transformed) according to the defined groups of samples and Bayesian posterior error analysis as implemented by Smyth (limma, version 3.4.5) (Smyth G K. Stat Appl Genet Mol Biol 2004; 3:Article3). Genes were considered significantly different if they met the following criteria: (i) average normalized signal intensity greater than four; (ii) logarithm [base 10] of odds [LOD] score greater than zero; and (iii) fold change greater than 1.5. All calculations and analyses were carried out using R (version 2.11.1) and Bioconductor computational tools (Gentleman R. Springer Science+Business Media 2005).

TABLE 12
Number of differentially expressed genes identified

| | | | Time | | | | | | | | | | |
|---|---|---|---|---|---|---|---|---|---|---|---|---|---|
| Animal | Dosing | | after last | | Whole | | | | | | | | |
| Model | Regimen | Comparison | dosing | | blood | Cerebellum | Cortex | Hippocampus | Striatum | Jenjunum | Kidney | Liver | Spleen |
| Naïve | Single | 100 mpk | 2 | h | 2 | 0 | 2 | 0 | 4 | 62 | 331 | 18 | 13 |
| | Dose | DMF-vs- | 7 | h | 0 | 0 | 2 | 2 | 2 | 44 | 93 | 5 | 2 |
| | | Vehicle | 12 | h | 5 | 0 | 0 | 0 | 2 | 37 | 100 | 1 | 0 |
| | | 100 mpk | 2 | h | 1 | 2 | 9 | 3 | 6 | 67 | 274 | 23 | 12 |
| | | MMF-vs- | 7 | h | 0 | 0 | 2 | 2 | 1 | 74 | 156 | 15 | 2 |
| | | Vehicle | 12 | h | 2 | 3 | 1 | 1 | 7 | 45 | 151 | 16 | 0 |
| | | 100 mpk | 2 | h | 0 | 0 | 0 | 2 | 0 | 1 | 1 | 0 | 0 |
| | | DMF-vs- | 7 | h | 1 | 0 | 0 | 0 | 0 | 2 | 1 | 1 | 0 |
| | | 100 mpk | 12 | h | 0 | 0 | 0 | 0 | 0 | 0 | 2 | 5 | 0 |
| | | MMF | | | | | | | | | | | |

TABLE 13
Number of differentially expressed genes identified

| | | | Time | | | | | | |
|---|---|---|---|---|---|---|---|---|---|
| Animal | Dosing | | after last | Whole | Brain | | Spinal | | Lymph |
| Model | Regimen | Comparison | dosing | blood | Hemi | Cerebellum | Cord | Spleen | Node |
| Naïve | Single | 100 mpk | 12 h | 5 | 3 | 8 | 1 | 4 | 4 |
| | Dose | DMF-vs- | | | | | | | |
| | | Vehicle | | | | | | | |
| | | 90 mpk | | 11 | 280 | 15 | 0 | 42 | 3 |
| | | MMF-vs- | | | | | | | |
| | | Vehicle | | | | | | | |
| | | 100 mpk | | 0 | 8 | 0 | 4 | 52 | 0 |
| | | DMF-vs- | | | | | | | |
| | | 90 mpk | | | | | | | |
| | | MMF | | | | | | | |

### Results and Discussion

### DMF- and MMF-Induced Expression Changes

Changes in gene expression were studied by applying a linear modeling approach to fit gene expression levels according to the defined groups of samples (e.g. DMF-, MMF-, and Vehicle-treated animals) and Bayesian posterior error analysis as implemented by Smyth (Smyth G K. Stat Appl Genet Mol Biol 2004; 3:Article3). The comparisons considered included: DMF-vs-MMF, DMF-vs-Vehicle, and MMF-vs-Vehicle. Differentially expressed genes (DEGs) were defined in each comparison as those Affymetrix probesets (henceforth referred to as “genes”) that exhibited an average normalized signal intensity greater than 4, a LOD score >0, and absolute fold change value greater than 1.5. TABLES 12 and 13 show the number of DEGs identified in each contrast. In general, more DEGs are apparent with the multi-dosing than the single dosing regimen, and no clear trend is seen between the 2 h, 7 h, and 12 h time points after a single dose. Very few DEGs are observed in blood and tissues derived from the central nervous system (brain, cerebellum, cortex, striatum, and spinal cord). The jejunum and kidney exhibited the highest number of DEGs in the animals receiving a single dose of treatment, whereas in the multi-dosed animals, the largest number of DEGs for both MMF and DMF treatments was consistently observed in the spleen.

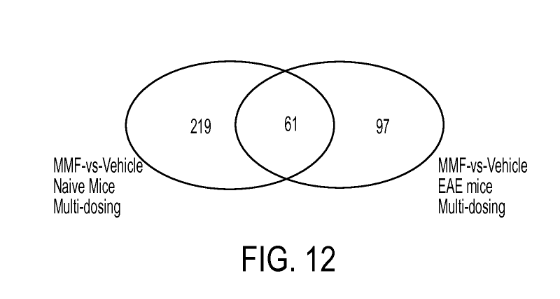

A large number of DEGs (280 DEGs) was observed in the brains of multi-dosed MMF animals as compared to the Vehicle treatment; DMF treatment did not induce this large effect. Interestingly, this same trend was seen in the brains of EAE animals chronically dosed with MMF (158 DEGs). These two sets of DEGs overlap by 61 genes, all of them down-regulated with MMF as compared to Vehicle treatment; this overlap is significant, with p-value 5.67×10⁻⁹⁴ (FIG. 12). The Ingenuity IPA software was used to perform pathway analysis on this common set of 61 MMF-induced genes, and 30 of the genes are affected in cancer.

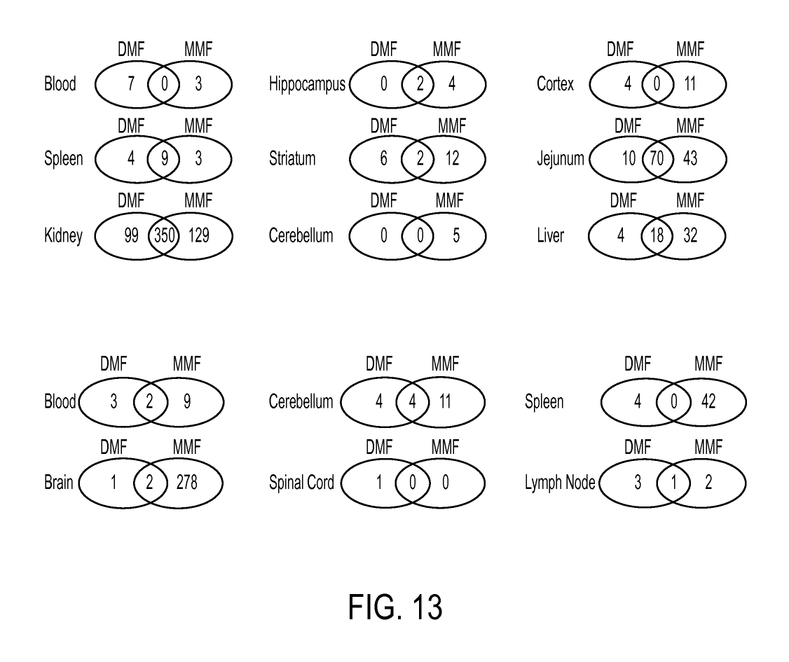

The gene expression results generally indicate that treatment of naive mice with DMF and MMF elicits unique pharmacodynamic responses in the tissues. FIG. 13 shows the number of DEGs that are in common and unique when the DMF- and MMF-treated animals were compared to the Vehicle-treated cohort. APPENDIX B and APPENDIX C provide lists of genes identified in naïve mice treated with single dose or multidose, respectively. It is clear that while there are some overlapping effects, there are many gene expression changes that are unique to either treatment. In the spleens of multi-dosed animals, for example, the expression level of a set of 52 genes is able to segregate the DMF-treated animals from the MMF-treated animals as shown in FIG. 14. Pathway analysis on these 52 genes using the Ingenuity IPA software suggests that IL2 and/or the NFkB complex may be activated in DMF-treated animals as compared to MMF-treated animals (Z-score 1.998 and 1.959, respectively). Four of these 52 genes, FCGR1A, ST18, CCL3L1, and VCAM1, are up-regulated and are downstream of NFkB activation. In addition, four genes amongst these 52 which are downstream of IL2 activation, CCR3, Klrb1c, NCR1, and CCL3L1, are also up-regulated.

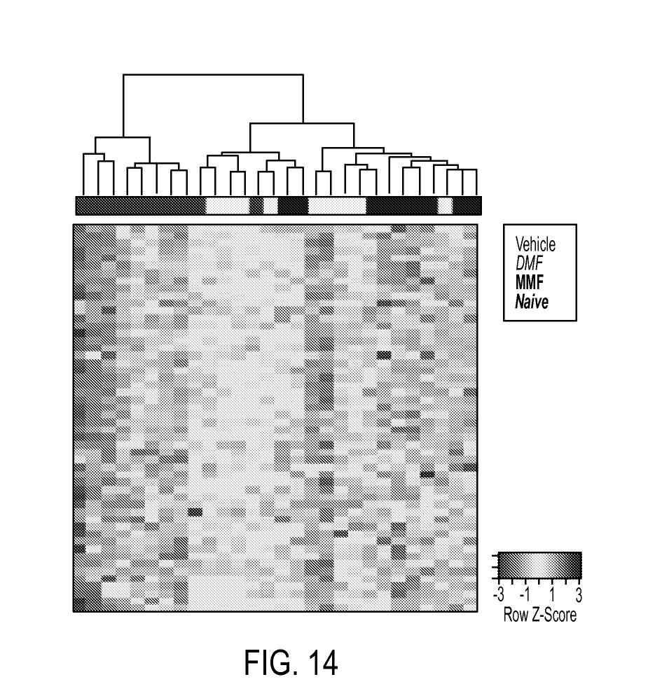

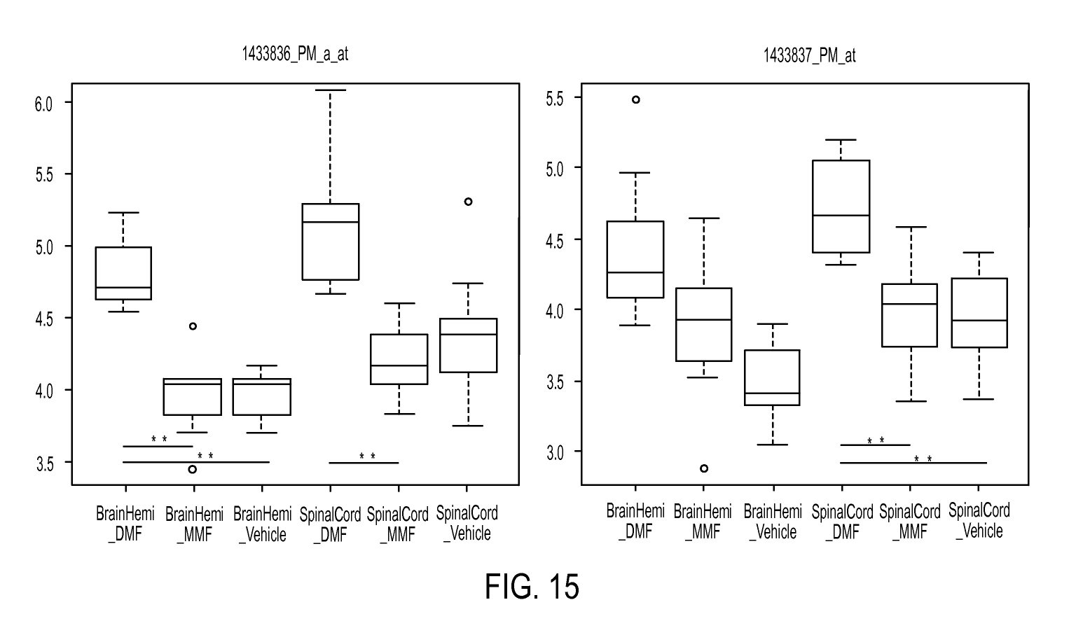

The DEPP gene is robustly induced with DMF but not with MMF in the brains and spinal cords of naïve mice that were administered a multi-dosing regimen of these compounds (TABLE 14 and FIG. 15). Two Affymetrix probe sets represent DEPP (1433836_PM_a_at and 1433837_PM_at), and both of these probe sets were significantly up-regulated in the spinal cords of animals multi-dosed with DMF as compared to Vehicle or MMF treatment. In the brain, the same trend was observed for one of the DEPP probe sets.

TABLE 14
DEPP is up-regulated in CNS tissues after multi-doing with DMF

| | | | | | DMF-vs-MMF | | | |
|---|---|---|---|---|---|---|---|---|
| | Affymetrix | Gene | Entrez | Avg | | p. | | |
| Tissue | ID | Symbol | Gene | NornInt | FC | value | lods | Significant |
| BrainHemi | 1433836_PM_a_at | Depp | 213393 | 4.26 | 1.78 | 0.00 | 7.17 | X |
| SpinalCord | 1433836_PM_a_at | Depp | 213393 | 4.56 | 1.92 | 0.00 | 3.02 | X |
| SpinalCord | 1433837_PM_at | Depp | 213393 | 4.21 | 1.65 | 0.00 | 0.66 | X |

| | DMF-vs-Vehicle | | | | MMF-vs-Vehicle | | | |
|---|---|---|---|---|---|---|---|---|
| | | p. | | | | p. | | |
| Tissue | FC | value | lods | Significant | FC | value | lods | Significant |
| BrainHemi | 1.77 | 0.00 | 7.48 | X | −1.01 | 0.94 | −7.24 | |
| SpinalCord | 1.66 | 0.00 | −0.48 | | −1.16 | 0.22 | −6.55 | |
| SpinalCord | 1.73 | 0.00 | 1.47 | X | 1.05 | 0.67 | −7.24 | |

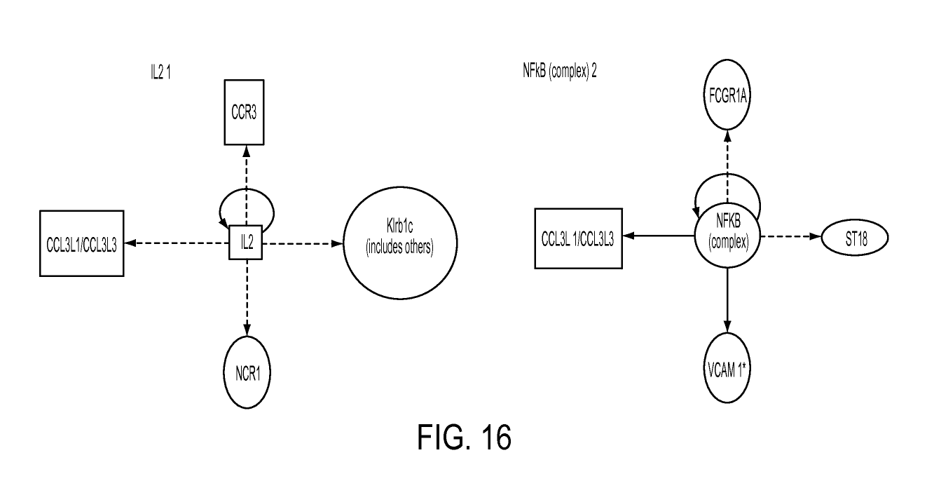

As shown in FIG. 16, IL21 or NFkB may be activated in the spleens of DMF-treated mice. A subset of 4 genes from the 52 DEGs that differentiate DMF from MMF treatment in the spleen suggest that IL2 or NFkB may be activated. Pathway analysis was performed in and figures were derived from the Ingenuity IPA software.

### Example 4

### Evaluation of EAE Mice Receiving DMF or MMF

As shown in Examples 1 and 3, a transcriptional comparison of single-dose MMF vs DMF in naïve mice revealed an NK cell “signature” in DMF-treated naïve mice. Example 2 describes FACS analysis which confirmed and extended the findings of an NK cell signature. The present example describes immunophenotyoing analysis of immune cell subsets in Experimental Autoimmune Encephalomyelitis (EAE) mice treated with a single dose or chronic administration of DMF or MMF.

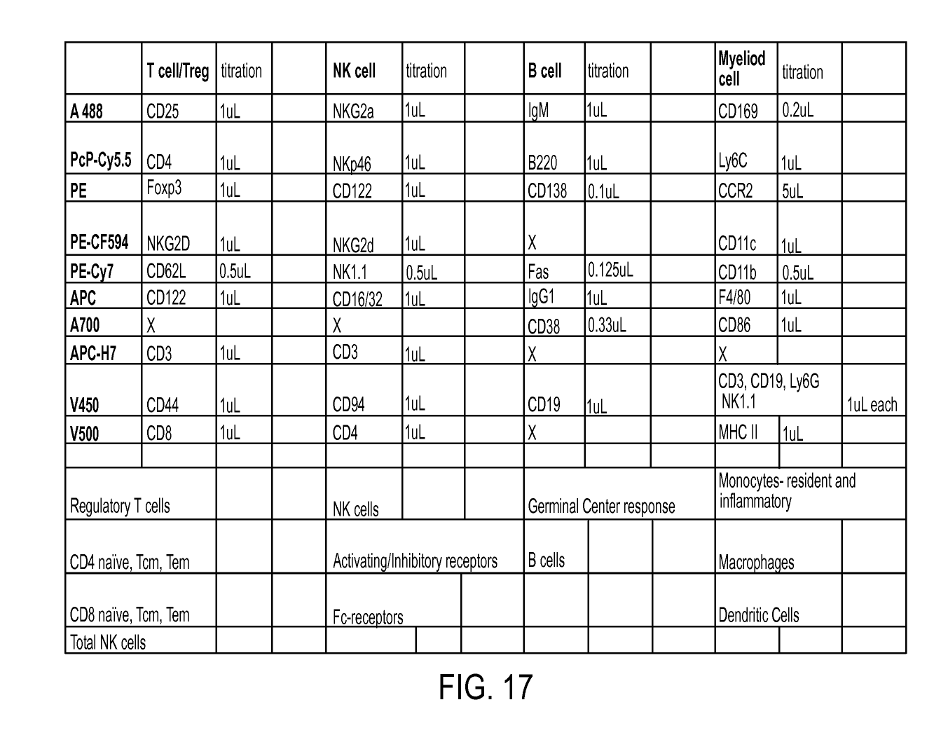

EAE induction is generally performed by immunization with brain extracts, CNS proteins (such as myelin basic protein), or peptides from such protein emulsified in an adjuvant such as complete Freund's adjuvant, e.g., as described in Linker et al., Brain. 2011 March 134(Pt3): 678-92. Vehicle, MMF or DMF was administered to EAE mice by a chronic or single dose administration, as described below Immune cells were obtained from various mouse tissues and analyzed by flow cytometry. FIG. 17 depicts exemplary immunophenotyping panels used to analyze various immune cell populations (e.g., T cells, T regulatory cells, NK cells, B cells, myeloid cells).

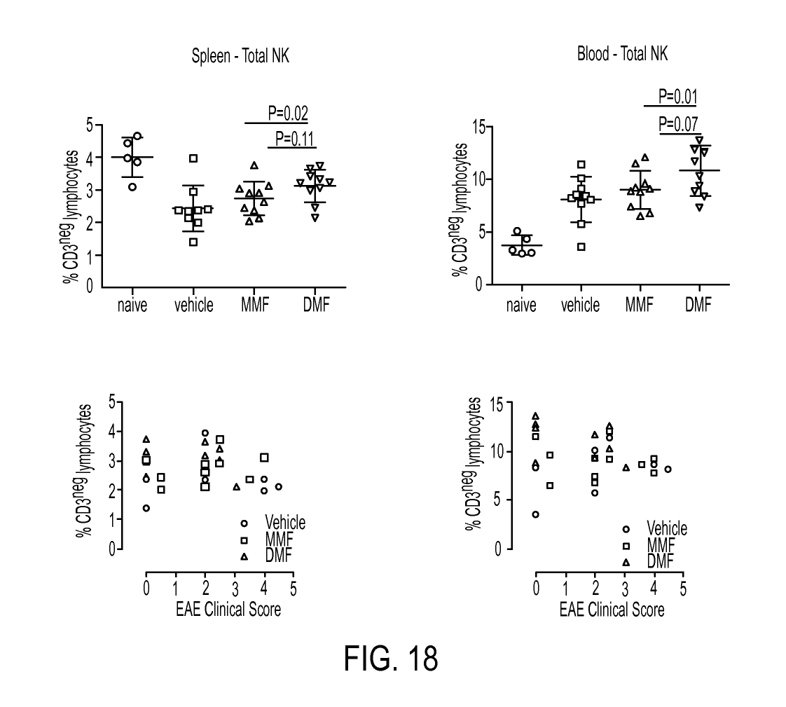

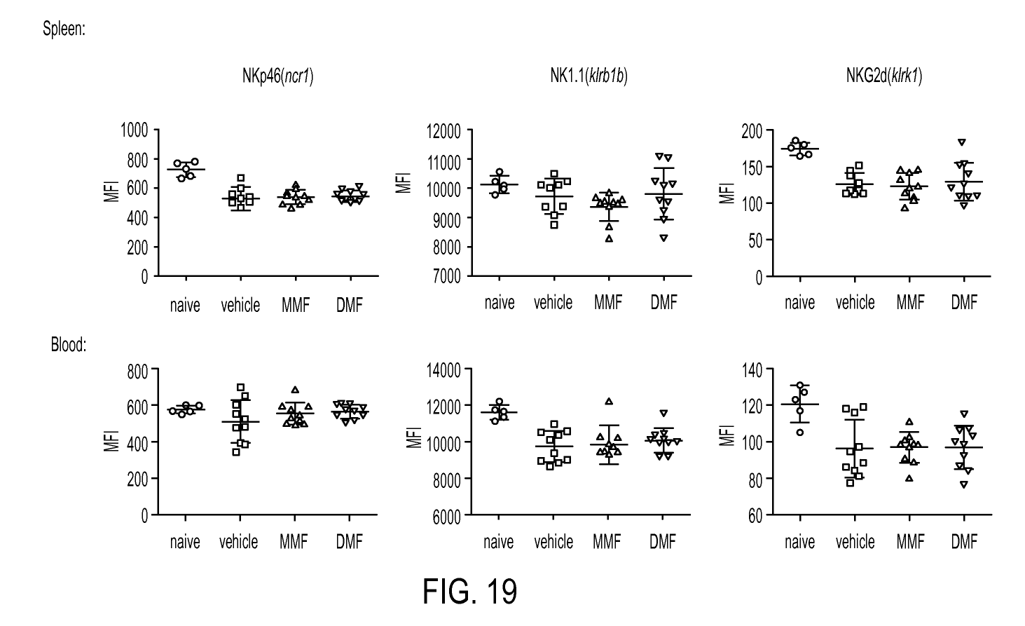

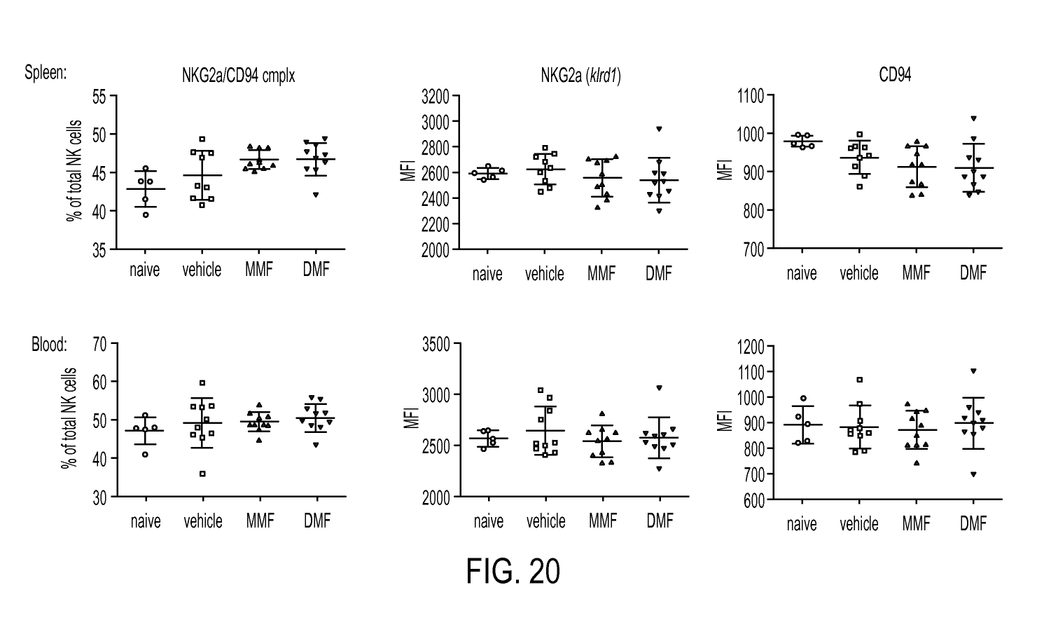

In the first study (chronic dose study), EAE mice were chronically dosed with vehicle, MMF, or DMF beginning at day 4 post-immunization. Mice were sacrificed on day 17 post-immunization at 12-hours after receiving the last dose. FIGS. 18-20 depict exemplary NK cell analysis in blood and spleen and EAE clinical score analysis for the chronic dosing experiment in EAE mice.

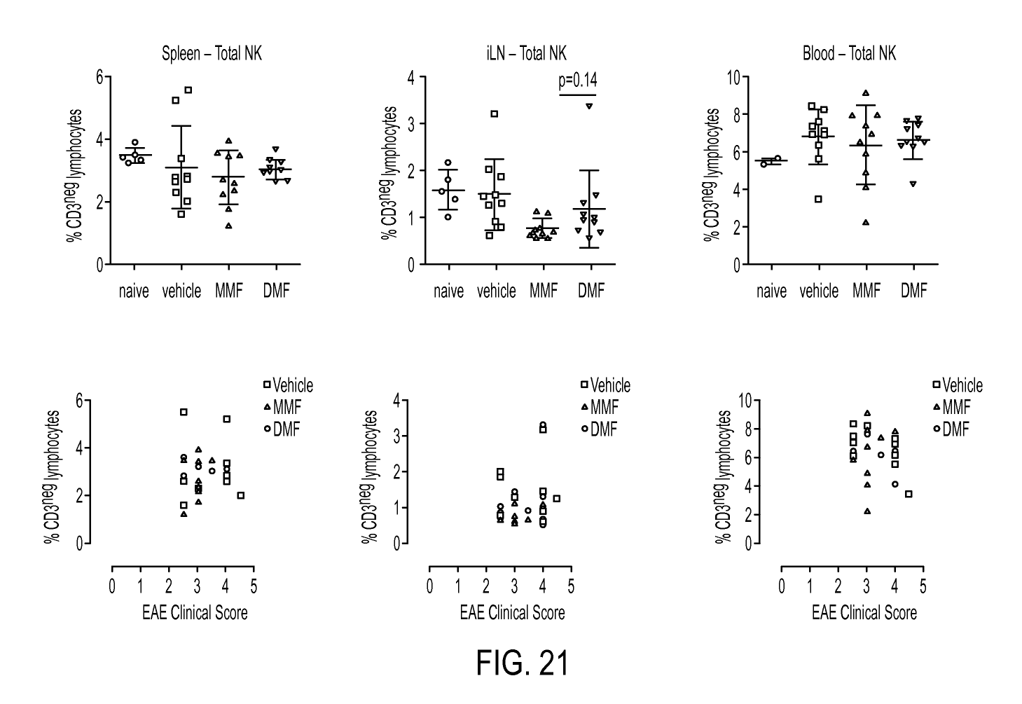

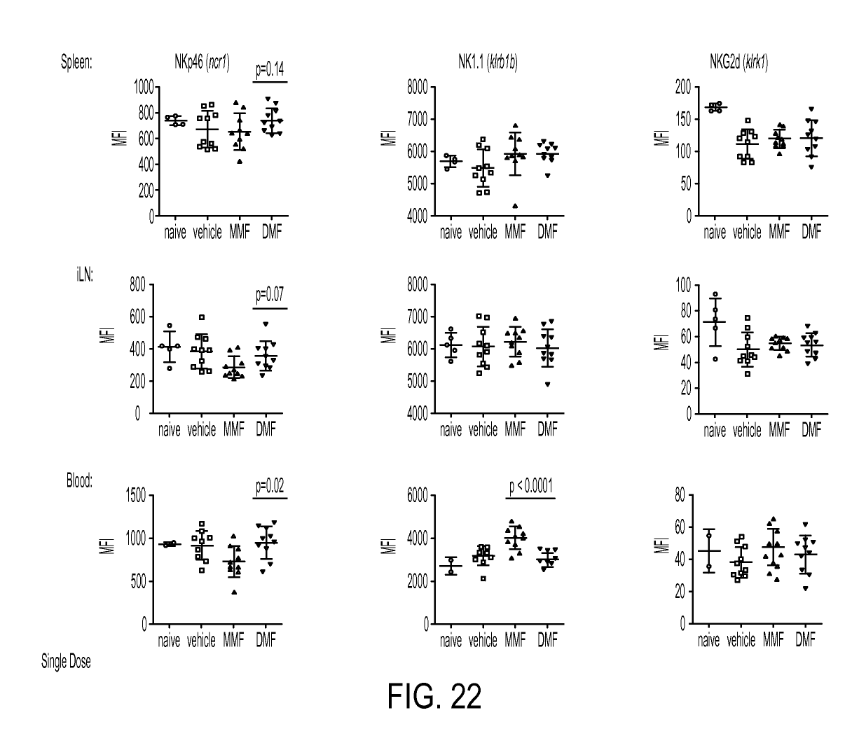

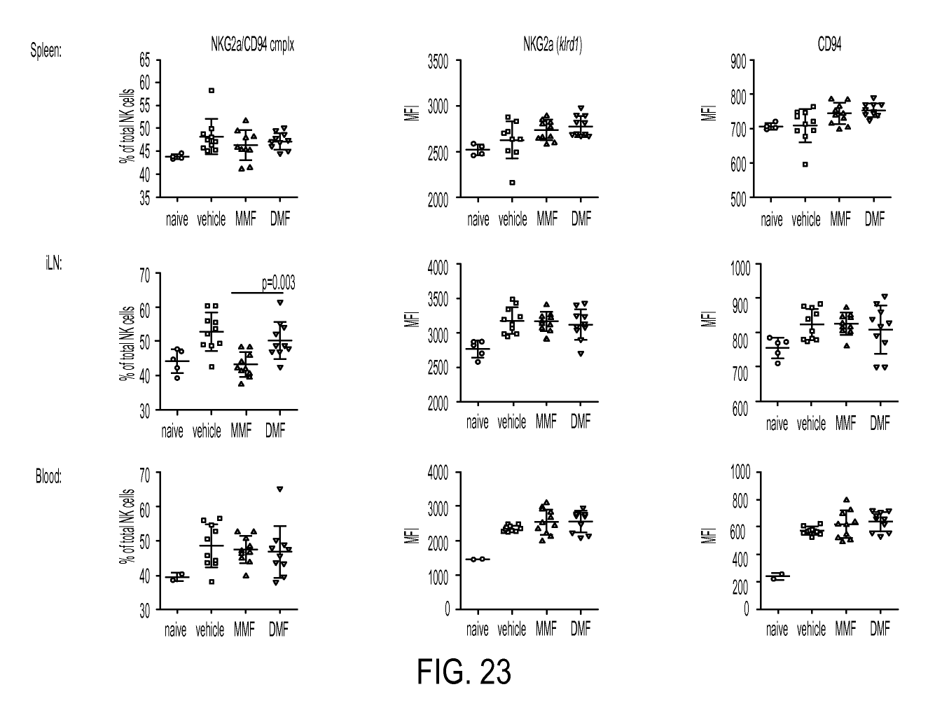

In the second study (single dose study), EAE mice were treated with a single dose of vehicle, MMF, or DMF on day 17 post-immunization. Animals were sacrificed 12 hours after receiving the dose. FIGS. 21-23 depict exemplary NK cell and NK subpopulation analysis in spleen, iLN, and blood for the single dose experiment in EAE mice.

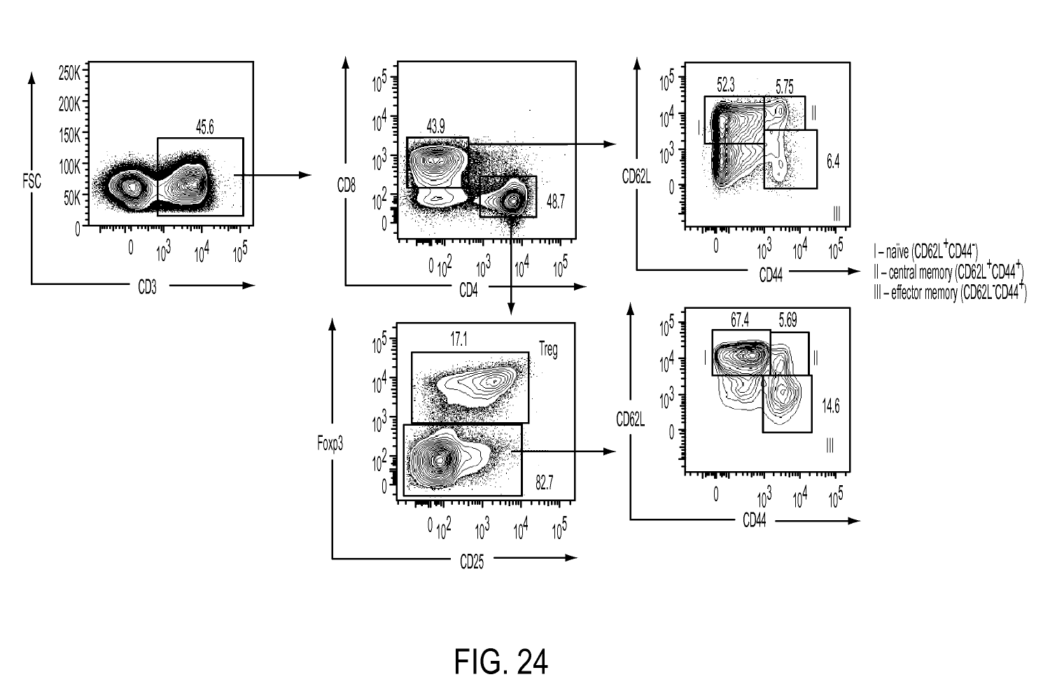

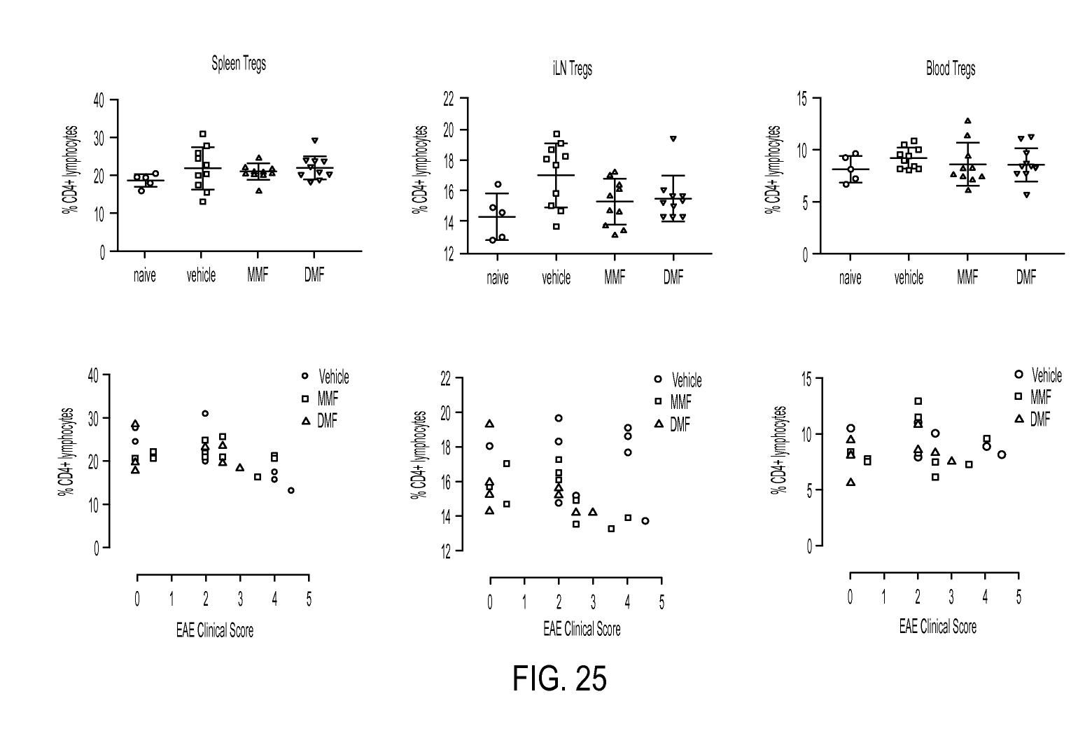

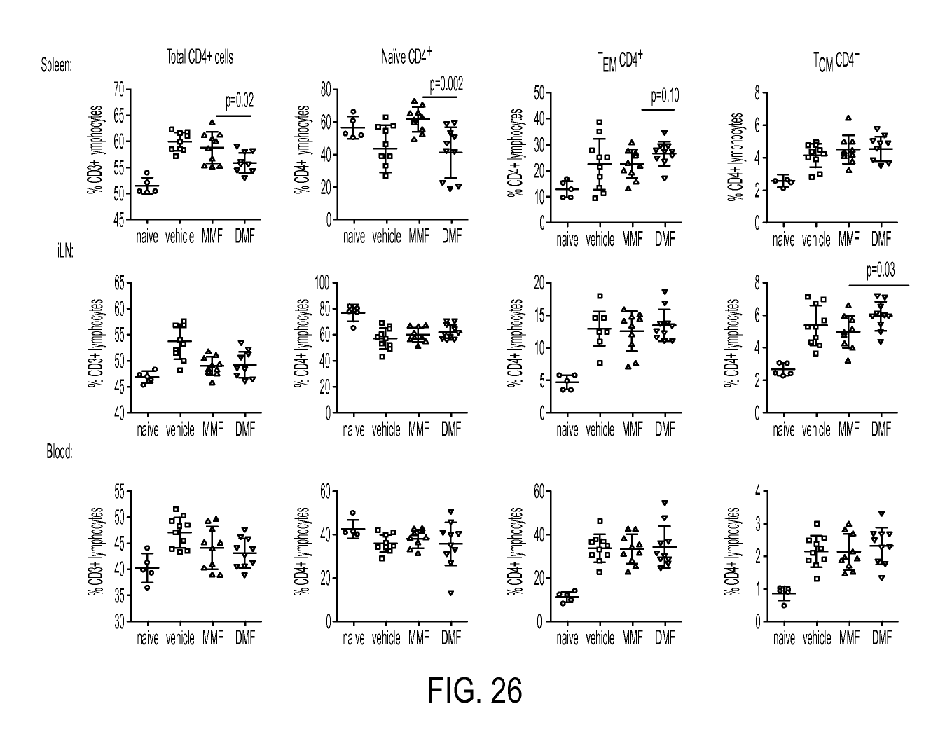

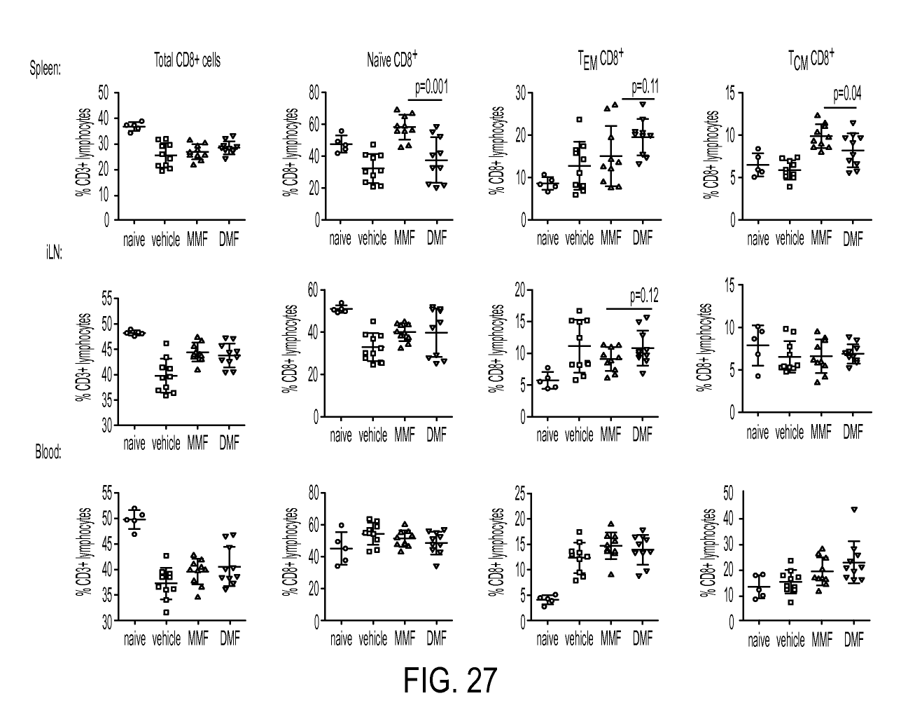

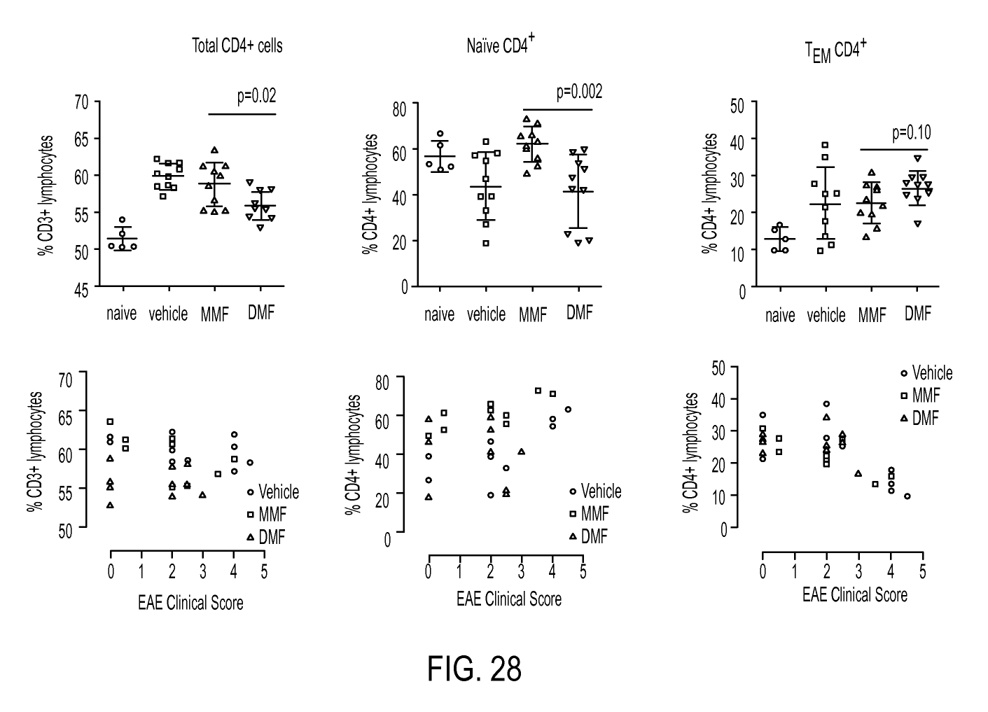

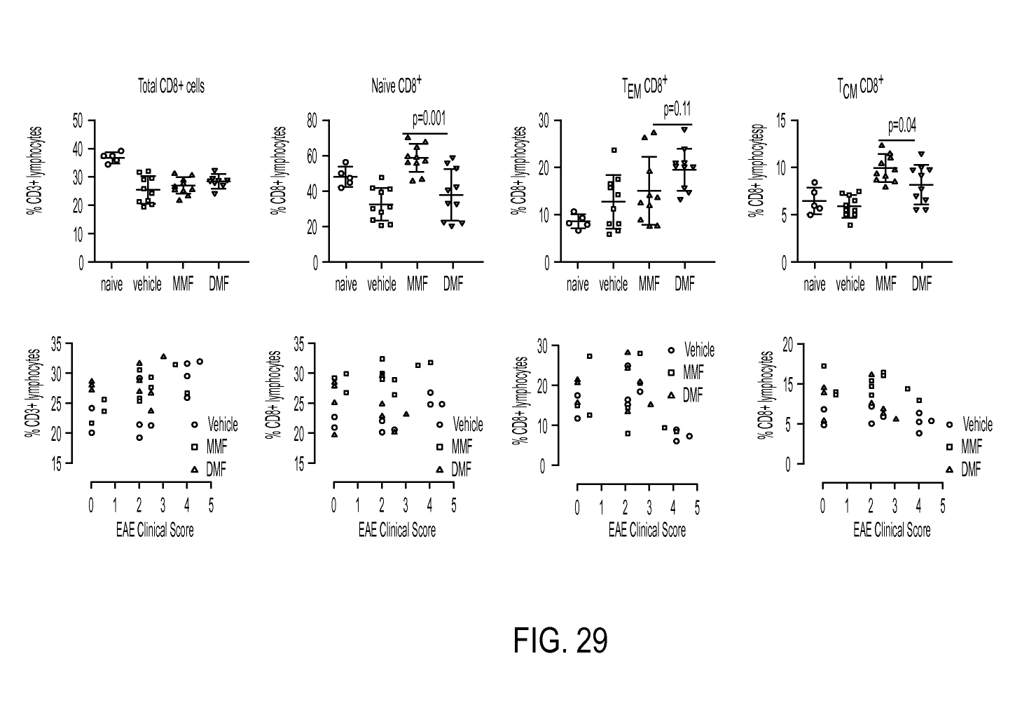

Additional immune cell types were analyzed, including T cells, B cells, and myeloid cells. In particular, T cells from EAE mice treated with vehicle, MMF, or DMF were analyzed by flow cytometry. FIG. 24 depicts an exemplary flow cytometry gating strategy for comparative analysis of DMF and MMF on T cell phenotype. Protein expression was quantified by mean fluorescent intensity (MFI). FIGS. 25-29 depict exemplary T cell and T cell subpopulation analysis in spleen, iLN and blood and EAE clinical score analyses for a chronic dosing experiment in EAE mice.

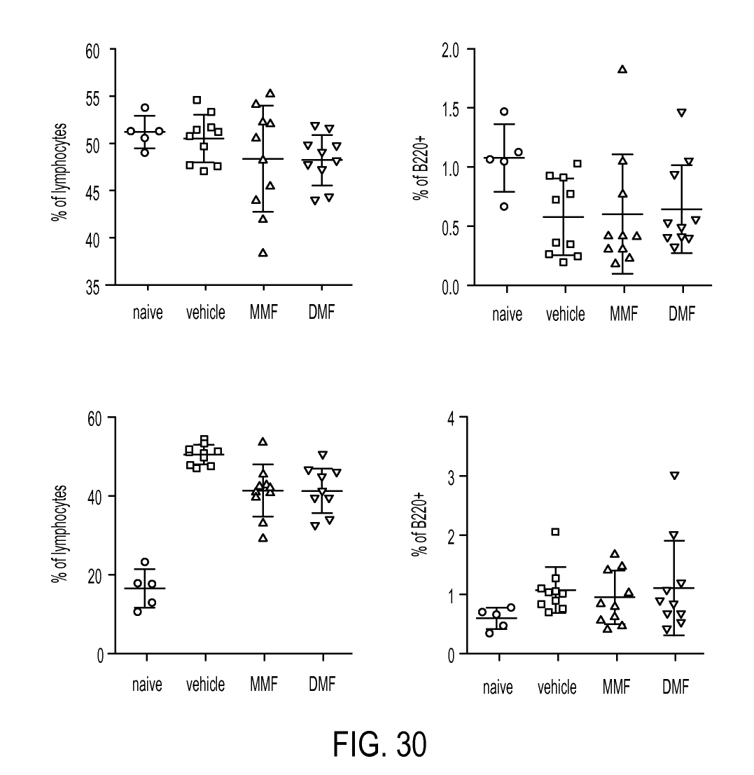

B cells from EAE mice treated with vehicle, MMF, or DMF were also analyzed by flow cytometry. FIG. 30 depicts exemplary B cell analysis in naïve, vehicle, MMF or DMF treated EAE mice.

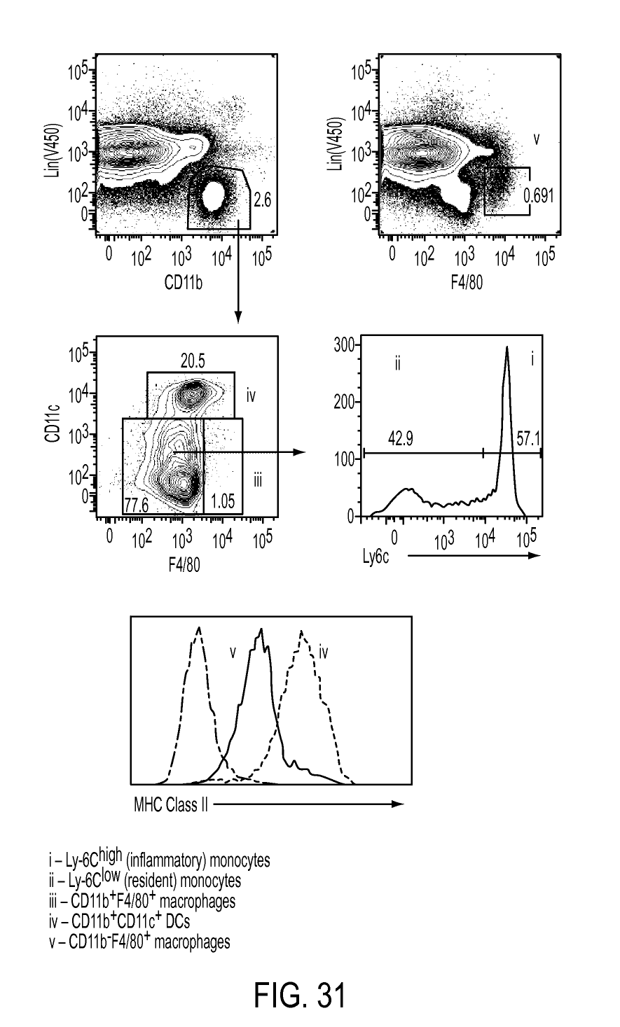

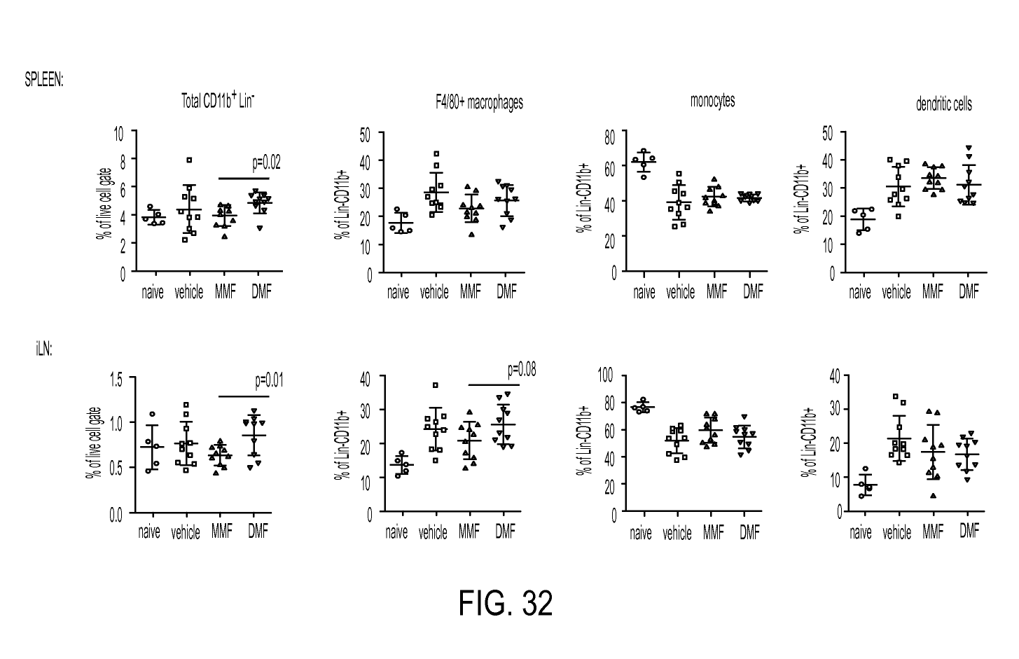

Myeloid cells from EAE mice treated with vehicle, MMF, or DMF were also analyzed by flow cytometry. FIG. 31 depicts an exemplary myeloid cell gating strategy for comparative analysis of DMF and MMF on myeloid cell phenotype. (Swirski, F. K. et al. Identification of splenic reservoir monocytes and their deployment to inflammatory sites. Science 325, 612-616 (2009)). FIG. 32 depicts exemplary myeloid cell subset analysis in spleen and iLN for a chronic dosing experiment in EAE mice.

### Example 5

### Transcriptional Profiling of Pharmacodynamic Effects of DMF and MMF in EAE Mice

This example demonstrates transcriptional profiling of pharmacodynamic effects of oral administration of DMF and MMF in single or multi-dose regiments in EAE mice. The number of independent mice whose tissues were harvested for transcript profiling studies and whose data passed QC is shown in TABLE 15.

TABLE 15
Number of replicates used in each treatment cohort for gene expression analyses.

| | | | Time | | | | | | | |
|---|---|---|---|---|---|---|---|---|---|---|
| Animal | Dosing | | after last | | Whole | | | Spinal | | Lymph |
| Model | Regimen | Treatment | dose | | Blood | Brain | Cerebellum | Cord | Spleen | Node |
| EAE | Chronic | 100 mpkDMF | 7 | h | 14 | 15 | 15 | 15(7) | 15 | 15 |
| | Dosing | | 12 | h | 13 | 14 | 15 | 15(8) | 15 | 15 |
| | | 90 mpkMMF | 7 | h | 10 | 14 | 13 | 14(8) | 13 | 12 |
| | | | 12 | h | 12 | 15 | 14 | 15(7) | 15 | 15 |
| | | Vehicle | 7 | h | 13 | 15 | 17 | 15(10) | 15 | 15 |
| | | | 12 | h | 8 | 12 | 13 | 13(9) | 13 | 13 |
| Naïve | | Naïve | 7 | h | 3 | 5 | 5 | 5 | 5 | 5 |
| | | | 12 | h | 3 | 5 | 5 | 5 | 5 | 5 |
| EAE | Acute | 100 mpkDMF | 7 | h | 12 | 15 | 15 | 15(0) | 15 | 15 |
| | Dosing | | 12 | h | 14 | 15 | 15 | 15 | 15(0) | 15 |
| | | 90 mpkMMF | 7 | h | 12 | 14 | 15 | 15(0) | 14 | 15 |
| | | | 12 | h | 11 | 15 | 15 | 15 | 15(0) | 15 |
| | | Vehicle | 7 | h | 12 | 15 | 17 | 15(0) | 14 | 15 |
| | | | 12 | h | 13 | 13 | 13 | 13 | 13(0) | 12 |
| Naïve | | Naïve | 7 | h | 3 | 5 | 5 | 5(0) | 4 | 5 |
| | | | 12 | h | 3 | 4 | 5 | 5 | 4(0) | 5 |

Numbers in parentheses indicate the number of samples included in a sub-analysis for which only the animals with the highest cumulative EAE score were used. Naïve mice do not have EAE nor were they administered any treatment.

### Materials and Methods

### Tissue Harvest

Animals were exposed to CO₂ and whole blood collected via cardiac puncture. Two 100 μl aliquots of whole blood were collected in 1.5 mL microcentrifuge tubes and snap frozen in liquid nitrogen. Peripheral (liver, spleen, kidney and jejunum) and CNS brain (cerebellum, hippocampus, striatum and cortex) tissues were harvested and snap frozen in liquid nitrogen, with special care taken to collect tissues of similar size and from the same location. All samples were stored at −80° C. until RNA extraction was conducted.

### Tissue RNA Extraction

For RNA preparation, frozen tissues were placed in 2 mL RNAse-free 96-well blocks with 1.5 mL QIAzol Lysis Reagent (QIAgen) and a 3.2 mm stainless steel bead (BioSpec Products, Bartlesville, Okla.). Tissues were disrupted for four cycles of 45 seconds in a Mini-Beadbeater (Biospec Products). RNA was extracted in chloroform and the aqueous phase was mixed with an equal volume of 70% ethanol. Extracted RNA was applied to RNeasy 96 plates and purified by the spin method according to the manufacturer's protocol (RNeasy 96 Universal Tissue Protocol, QIAgen, Hilden Germany).

Blood RNA Extraction and QC (Allaire N E, et al. BMC Res Notes. 2013 Jan. 5; 6:8)

50 ul of snap frozen mouse blood was re-suspended in lysis buffer with proteinase K and arrayed into deep-well plates for automated RNA extraction. RNA extractions were completed on Arrayplex (Beckman Coulter, Indianapolis, Ind.) using Agencourt RNAdvance Blood kit (Part number A35604) according to the manufacturer's specifications. RNA integrity was assessed using the HT RNA reagent kit (Part number 760410, Caliper Life Sciences, Hopkinton, Mass.) using a LabChip GX (PerkinElmer, Waltham, Mass.). RNA samples with a RQS score of >8.0 were considered high quality for downstream microarray processing.

### Sample Labeling, Hybridization and Scanning

Automated sample amplifications and biotin labelings were carried out using the NuGEN Ovation RNA Amplification system V2 (Cat #3100), Ovation WB reagent (Cat #1300) and Encore Biotin module (Cat #4200) (NuGEN Technologies, Inc, San Carlos, Calif.) according to manufacturer's recommendations using an Arrayplex automated liquid handler (Beckman Coulter, Indianapolis, Ind.). 2 ug of biotin labeled sscDNA probe were hybridized to Affymetrix HT_MG-430_PM plate arrays with modified conditions as described in Allaire et al. Washing and staining of the hybridized arrays were completed as described in the GeneChip Expression analysis technical manual for HT plate arrays using the Genechip® Array Station (Affymetrix, Santa Clara, Calif.) with modifications as described in Allaire et al. The processed GeneChip® plate arrays were scanned using GeneTitan scanner (Affymetrix, Santa Clara, Calif.).

### Analysis of Affymetrix Data

Affymetrix scans were subject to quality control (QC) measures. These tests included a visual inspection of the distribution of raw signal intensities and an assessment of RNA degradation, relative log expression (RLE), and normalized unscaled standard error (NUSE). All sample scans that passed these QC metrics were included in the analysis.

All CEL files were subjected to GC-content-based Robust Multi-array Average (GCRMA) normalization (version 2.20.0) (Irizarry R A, et al. Nucleic Acids Res 2003; 31:e15; Li C, et al. Genome Biol 2001; 2:RESEARCH0032). Expression levels were log (base 2) transformed.

Analyses were applied to discover genes that were differentially expressed (DEGs) between different animal treatments. The contrasts carried out are shown in TABLE 16. To identify differentially expressed genes between groups of samples, we applied the linear modeling approach (ANOVA) to fit gene expression levels (log 2 transformed) according to the defined groups of samples and Bayesian posterior error analysis as implemented by Smyth (limma, version 3.4.5) (Smyth G K. Stat Appl Genet Mol Biol 2004; 3:Article3). Genes were considered significantly different if they met the following criteria: (i) average normalized signal intensity greater than four; (ii) logarithm [base 10] of odds [LOD] score greater than zero; and (iii) fold change greater than 1.5. All calculations and analyses were carried out using R (version 2.11.1) and Bioconductor computational tools (Gentleman R. Springer Science+Business Media 2005).

TABLE 16
Number of differentially expressed genes identified

| | | | Time | | | | | | | |
|---|---|---|---|---|---|---|---|---|---|---|
| Animal | Dosing | | after last | | Whole | | | Spinal | | Lymph |
| Model | Regimen | Comparison | dosing | | Blood | Brain | Cerebellum | Cord | Spleen | Node |
| EAE | Chronic | 100 mpkDMF- | 7 | h | 8 | 6 | 0 | 3 | 12 | 14 |
| | Dosing | vs-Vehicle | 12 | h | 8 | 0 | 0 | 3 | 7 | 8 |
| | | 90 mpkMMF- | 7 | h | 19 | 158 | 2 | 7 | 23 | 21 |
| | | vs-Vehicle | 12 | h | 7 | 0 | 3 | 2 | 9 | 7 |
| | | 100 mpkDMF- | 7 | h | 0 | 31 | 2 | 2 | 3 | 2 |
| | | vs-90 mpkMMF | 12 | h | 0 | 0 | 3 | 40 | 4 | 6 |
| | | Vehicle-vs-Naïve | 7 | h | 1782 | 252 | 185 | ND | 3987 | 3893 |
| | | | 12 | h | 1265 | 143 | 69 | ND | 3013 | 1145 |
| EAE | Acute | 100 mpkDMF- | 7 | h | 3 | 0 | 0 | ND | 21 | 7 |
| | Dosing | vs-Vehicle | 12 | h | 0 | 0 | 0 | 1 | ND | 2 |
| | | 90 mpkMMF- | 7 | h | 6 | 0 | 1 | ND | 114 | 6 |
| | | vs-Vehicle | 12 | h | 7 | 0 | 1 | 1 | ND | 4 |
| | | 100 mpkDMF- | 7 | h | 0 | 0 | 0 | ND | 0 | 0 |
| | | vs-90 mpkMMF | 12 | h | 0 | 0 | 0 | 0 | ND | 0 |
| | | Vehicle-vs-Naïve | 7 | h | 1748 | 459 | 473 | ND | 3098 | 5333 |
| | | | 12 | h | 1164 | 514 | 761 | 6850 | ND | 1710 |

ND indicates “Not Done” due to heterogeneity of EAE scores that manifest in global transcript profiling.

### Results and Discussion

### Global Gene Expression Patterns Define Distinct EAE Subsets

In order to understand if global gene expression patterns might arise in each tissue from experimental treatments, the set of Affymetrix probesets (henceforth referred to as “genes”) that exhibited an average normalized intensity greater than 4 within a treatment group and with coefficient of variation (CV) greater than 0.05 was subjected to unsupervised clustering. Generally, no distinct sample groupings were apparent from this analysis; however, in the following cohorts, differences in EAE severity across the mice were manifest in global gene expression patterns:

### Spinal Cord, Acute Dosing, 7 h

### Spinal Cord, Chronic Dosing, 7 h

### Spinal Cord, Chronic Dosing, 12 h

### Spleen, Acute Dosing, 12 h

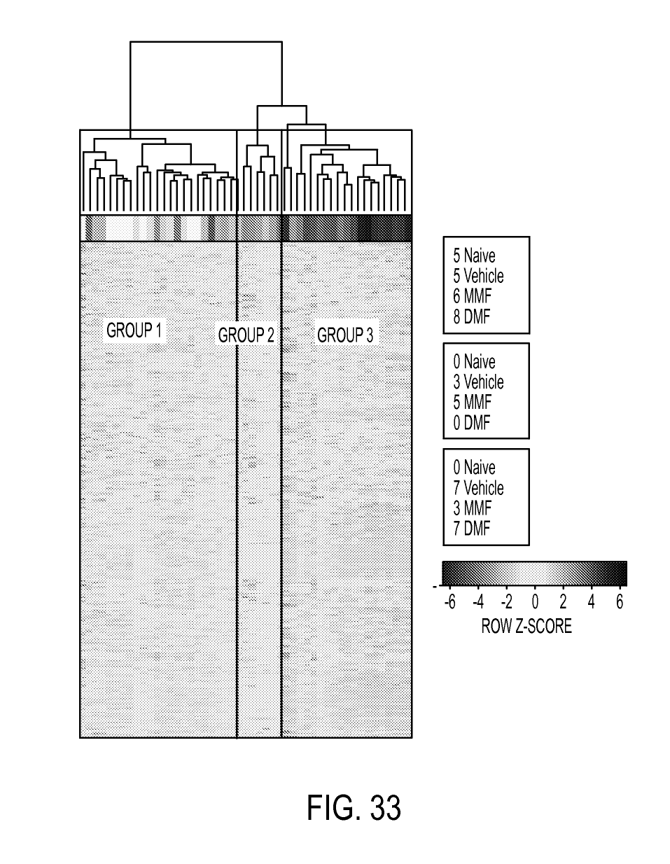

An example of this animal grouping is shown in FIG. 33 for the spinal cord from the chronically-dosed 7 h EAE mice. For this cohort, a set of 2,872 genes remained after the filtering method outlined above. Three distinct groups of mice result from unsupervised clustering, and these animal groups correlate with the cumulative EAE score. These results indicate that in the spinal cord and spleen, global gene expression changes occur, and these changes are likely reflective of the disease severity of the animal. Subsequent analyses for the cohorts listed above only included those animals with the most severe EAE scores; however, for the cohorts ‘Spinal Cord, Acute Dosing, 7 h’ and ‘Spleen, Acute Dosing, 12 h,’ there were an insufficient number of mice in the different treatment groups to perform such a sub-analysis.

### DMF- and MMF-Induced Expression Changes

Changes in gene expression were studied by applying a linear modeling approach to fit gene expression levels according to the defined groups of samples (e.g. DMF-, MMF-, and Vehicle-treated animals) and Bayesian posterior error analysis as implemented by Smyth (Smyth G K. Stat Appl Genet Mol Biol 2004; 3:Article3). The comparisons considered included: DMF-vs-MMF, DMF-vs-Vehicle, MMF-vs-Vehicle, and Vehicle-vs-Naïve. Differentially expressed genes (DEGs) were defined in each comparison as those genes that exhibited an average normalized signal intensity greater than 4, a LOD score >0, and absolute fold change value greater than 1.5. TABLE 16 shows the number of DEGs identified in each contrast. In general, more DEGs are apparent with chronic dosing than an acute dosing regimen, and no clear trend was seen between the 7 h and 12 h time points in either dosing regimen. Very few DEGs were observed in blood and tissues derived from the central nervous system (brain, cerebellum, and spinal cord). The lymph node and spleen exhibited the highest number of DEGs.

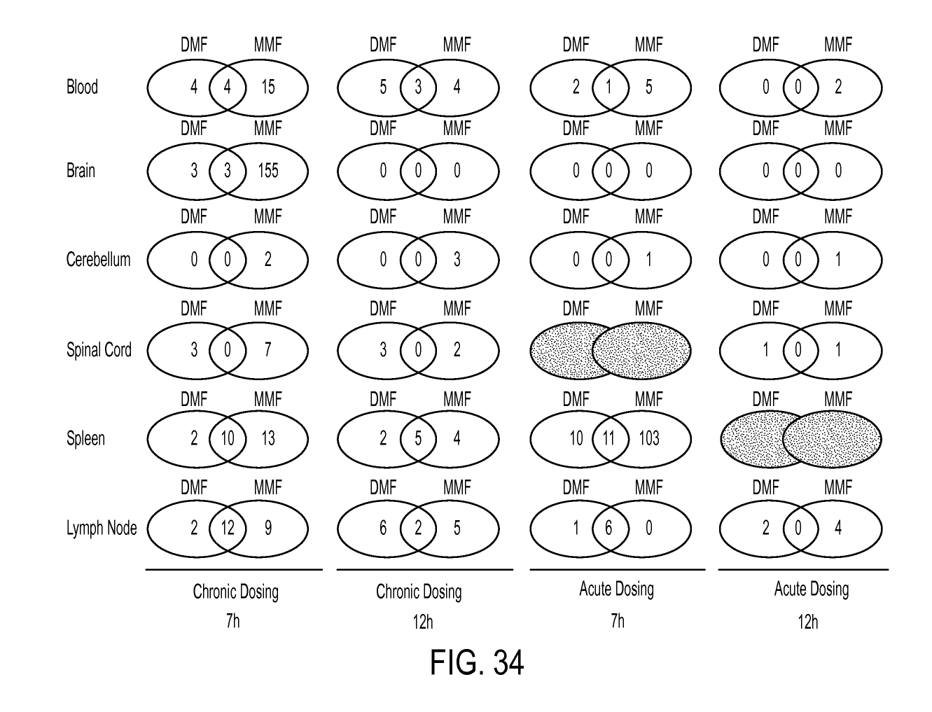

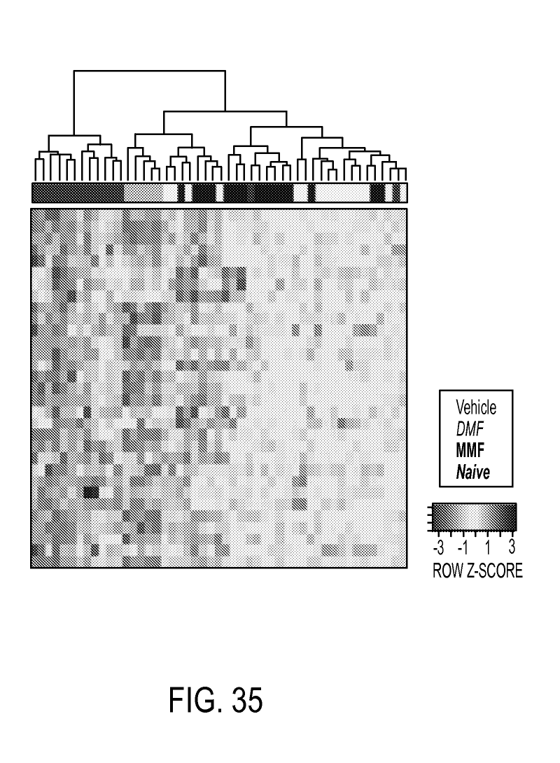

The gene expression results indicate that treatment of EAE mice with DMF and MMF elicits unique pharmacodynamic responses in the tissues. FIG. 34 shows the number of DEGs that are in common and unique when the DMF- and MMF-treated animals were compared to the Vehicle-treated cohort. APPENDIX D and APPENDIX E provide lists of genes identified in EAE mice treated with single dose or multidose, respectively. It is clear that while there are some overlapping effects, there are many gene expression changes that are unique to either treatment. The direct comparison of gene expression in the brains of chronically-dosed DMF and MMF animals yielded one of the larger DEG lists. It is clear, as shown in FIG. 35 using unsupervised clustering, that the expression level of these 31 genes can segregate the DMF-treated animals from the MMF-treated animals.

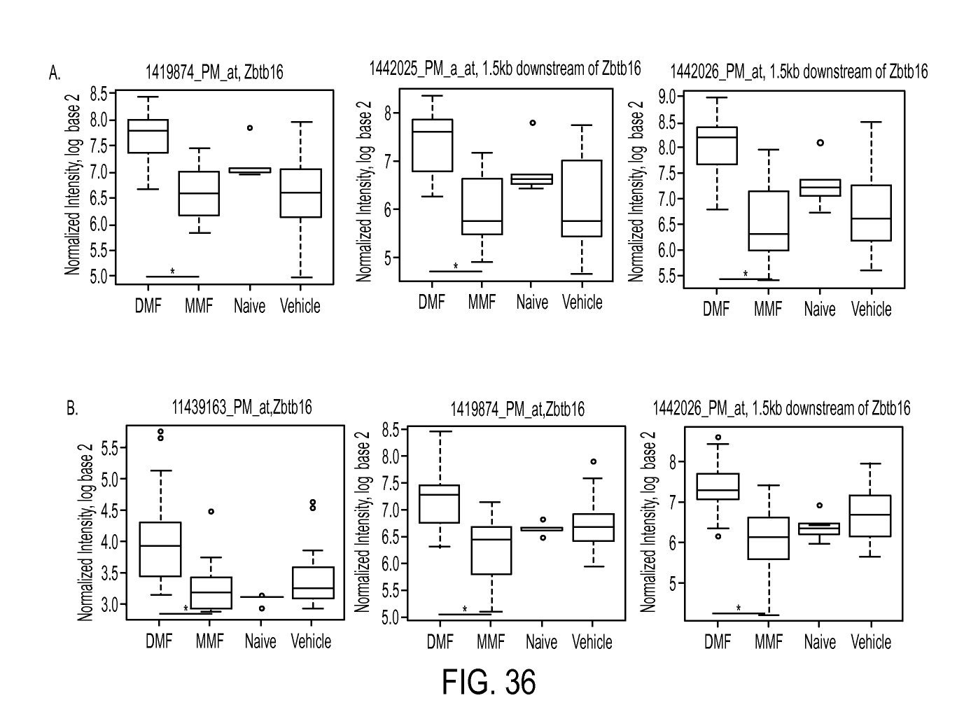

Finally, several Affymetrix probe sets that represent the Zbtb16 transcript exhibited increased signal in DMF-treated animals as compared to the MMF-treated animals in the lymph node and spleen (FIG. 36). Interestingly, probe sets positioned just 1.5 kb downstream of the annotated end of the Zbtb16 3′UTR also exhibited this trend, suggesting that a unique isoform of Zbtb16 might be expressed in DMF-treated EAE mice.

### Example 6

### Preparation of Compounds (III)-(VI)

The compounds of Formulae (III)-(VI) may be prepared using methods known to those skilled in the art, or the methods disclosed in the present invention.

Specifically, the compounds of this invention of Formula IV may be prepared by the exemplary reaction in Scheme 1.

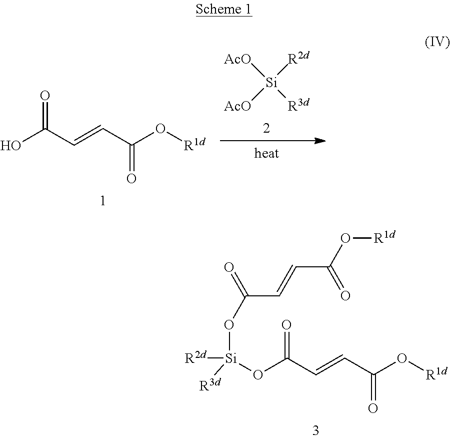

wherein R^{1d}, R^{2d}, and R^{3d} are each defined above for Formula IV.

Reaction of fumaric acid ester 1 with silane diacetate intermediate 2 in a refluxing organic solvent such as diethyl ether, toluene, or hexane to give the desired siloxane 3.

Some of the fumaric acid esters 1 are commercially available. Fumaric acid ester 1′ can be prepared, for example, using synthetic methods known by one of ordinary skill in the art. For example, fumaric acid can be converted by reacting alcohol (R^{1c}—OH) with a catalytic amount of p-toluene sulfonic acid at room temperature for a few hours to overnight as shown in Scheme 2.

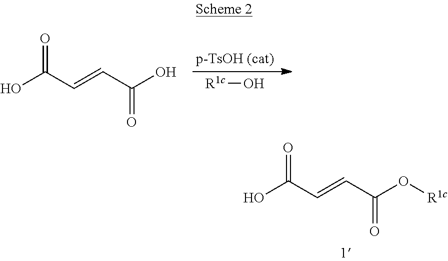

wherein R^{1c} is defined above for Formula III.

Alternatively, fumaric acid ester 1′ can be prepared by reacting alcohol (R^{1c}—OH) under the coupling conditions of hydroxybenzotriazole (HOBT), 1-ethyl-3-(3-dimethylaminopropyl)carbodiimide (EDCI), and diisopropyl amine (DIPEA) as shown in Scheme 3.

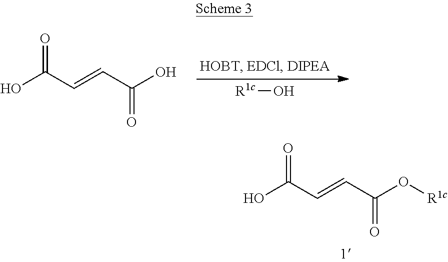

wherein R^{1c} is defined above for Formula III.

Some of the silanes that can be used in the present invention are commercially available. Commercially available silyl halides include trimethylsilyl chloride, dichloro-methylphenylsilane, dimethyldichlorosilane, methyltrichlorosilane, (4-aminobutyl)diethoxymethylsilane, trichloro(chloromethyl)silane, trichloro(dichlorophenyl)silane, trichloroethylsilane, trichlorophenylsilane, and trimethylchlorosilane. Commercial sources for silyl halides include Sigma Aldrich and Acros Organics.

Silanes used in the present invention can be prepared, for example, using synthetic methods known by one of ordinary skill in the art. For example, trichlorosilane may be prepared by the exemplary reaction in Scheme 4.

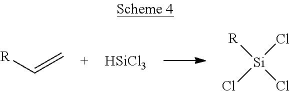

The silylation of styrene derivatives catalyzed by palladium is described in Zhang, F. and Fan, Q.-H., Organic & Biomolecular Chemistry 7:4470-4474 (2009) and in Bell, J. R., et al., Tetrahedron 65:9368-9372 (2009).

Diacetate intermediate 2 may be prepared by treatment of dichlorosubstituted silicon compound 4 with sodium acetate in diethyl ether under reflux as shown in Scheme 5.

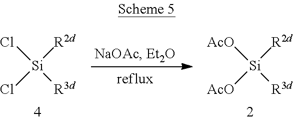

wherein R^{2d} and R^{3d} are each defined above for Formula IV.

Specifically, the compounds of this invention of Formula V may be prepared by the exemplary reaction in Scheme 6.

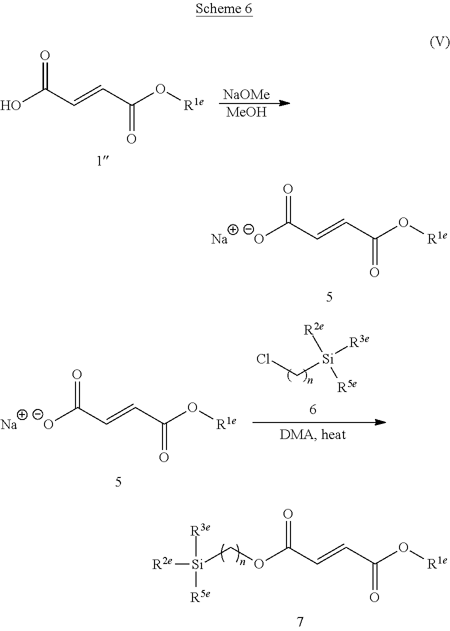

wherein R^{1e}, R^{2e}, R^{3e}, and R^{5e} are as defined above for Formula V.

Fumaric acid ester 1″ can be converted to the sodium salt 5 using, for example, sodium methoxide in methanol at room temperature. Removal of the solvent would afford sodium salt 5. Treatment of the sodium salt 5 with silane 6 in an organic solvent such as dimethylformamide under reflux would generate ester 7. The synthesis of structurally related (trimethoxysilyl)-methyl esters is described in Voronkov, M. G., et al., Zhurnal Obshchei Khimii 52:2052-2055 (1982).

Alternatively, the compounds of this invention of Formula V may be prepared by the exemplary reaction in Scheme 7.

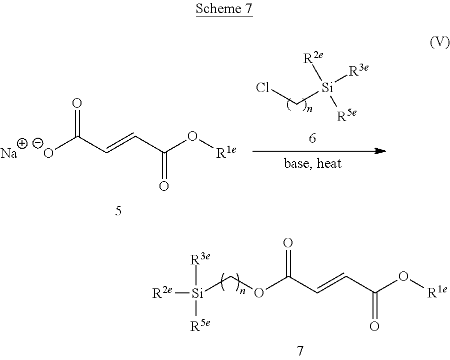

wherein R^{1e}, R^{4e}, R^{5e}, R^{6e}, and n are as defined above for Formula V.

Treatment of the sodium salt 5 with silane 6 in an organic solvent such as dimethylformamide under heating with or without an acid scavenger would generate ester 7.

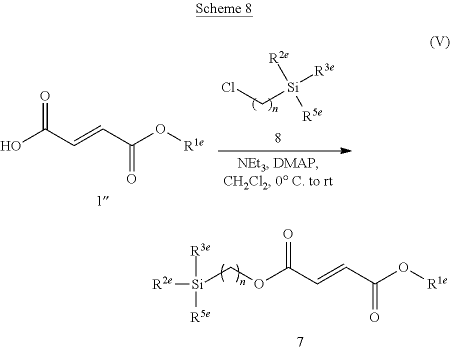

wherein R^{1e}, R^{4e}, R^{5e}, R^{6e}, and n are as defined above for Formula V.

Reaction of fumaric acid ester 1″ with tri-substituted silane alcohol 8 in methylene chloride with mild base such as triethyl amine and 4-N,N-dimethyl amino pyridine (DMAP) at room temperature generates fumarate 7. See Coelho, P. J., et al., Eur. J. Org. Chem. 3039-3046 (2000).

Specifically, the compounds of this invention of Formula VI can be prepared by the exemplary reaction in Scheme 9.

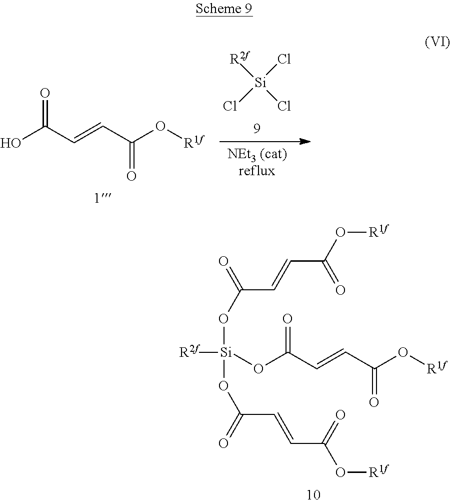

wherein R^{1f} and R^{2f} are as defined above for Formula VI.

Reaction of fumaric acid 1′″ with trichlorosilane 9 in a refluxing organic solvent such as hexane or toluene using a catalytic amount of a base such as triethylamine generates the trifumarate silane 10. The reaction of acetic and methacrylic acids with 1-silyladamantanes is described in Fedotov, N. S., et al., Zhurnal Obshchei Khimii 52:1837-1842 (1982).

### Example 7

### Synthesis of (E)-O,O′-(dimethylsilanediyl)dimethyl difumarate (Compound 11)

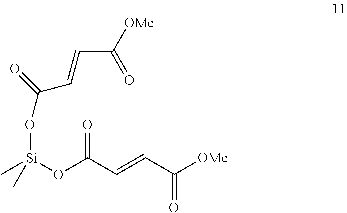 [SMILES: COC(=O)/C=C/C(=O)O[Si](C)(C)OC(=O)/C=C/C(=O)OC]

### Step 1: Preparation of Dimethylsilanediyl Diacetate 11B

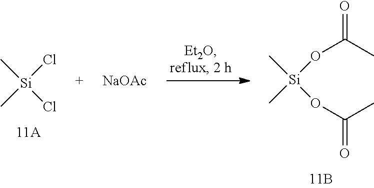

To a slurry of sodium acetate (8.2 g, 100 mmol, 2.0 equiv.) in anhydrous diethyl ether (40 mL) was slowly added a solution of dimethyldichloro silane 11A (6.45 g, 50 mmol, 1.0 equiv.) in anhydrous diethyl ether (10 mL). After addition was completed, the mixture was heated at reflux for 2 hours, and then filtered under N₂. The filtrate was concentrated under vacuum at 40° C. to give diacetate 11B as a colorless oil (6.1 g, 70%). ¹H NMR (400 MHz, CDCl₃) δ ppm: 2.08 (s, 6H), 0.48 (s, 6H).

### Step 2: Preparation of (E)-O,O′-(dimethylsilanediyl)dimethyl difumarate 11

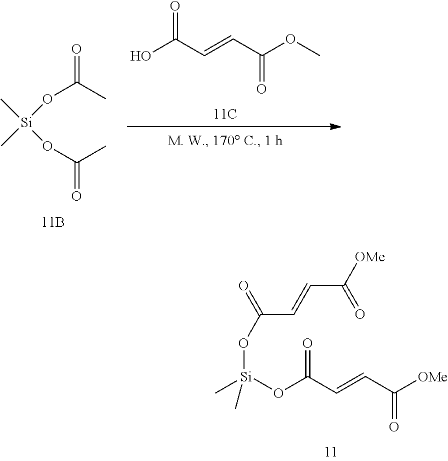

A mixture of 11B (2.0 mL, 12 mmol, 1.5 equiv.) and 11C (1.04 g, 8.0 mmol, 1.0 equiv.) in a sealed tube was heated at 170° C. with stirring under microwave condition for 1 hour. After cooling to 50° C., the mixture was transferred to a round bottom flask and the excess silica reactant 11B was removed under vacuum at 100° C. to afford compound 11 as brown oil (1.47 g, 60%). ¹H NMR (400 MHz, CDCl₃) δ ppm: 6.82-6.80 (m, 4H), 3.79 (s, 6H), 0.57 (s, 6H).

### Example 8

### Synthesis of methyl ((trimethoxysilyl)methyl)fumarate (Compound 12)

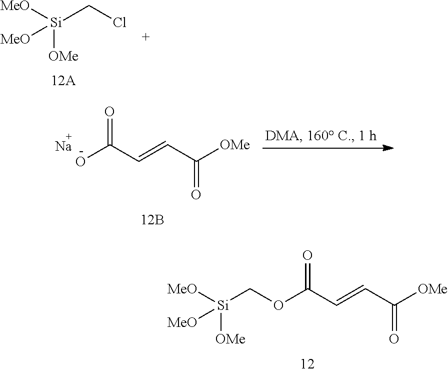

To a stirred solution of monomethyl fumarate (3.5 g, 27 mmol, 1.0 equiv.) in anhydrous THF (35 mL) at room temperature was added sodium hydride (1.08 g, 27 mmol, 1.0 equiv.) in small portions. After addition, the mixture was heated to reflux for 3 hours, and then cooled to room temperature. The solid was collected by filtration and washed twice with diethyl ether, and further dried in vacuo to give 3.8 g of 12B (93%).

To a suspension of 12B (760 mg, 5.0 mmol, 1.0 equiv.) in dry DMA (5 mL) at 100° C. under nitrogen was added a solution of 12A (1.03 g, 6.0 mmol, 1.2 equiv.) in dry DMA (1 mL) dropwise. The resulting mixture was heated to 160° C. and stirred for 1 hour, and then cooled to room temperature. The solid was filtered, and the filtrate was evaporated under reduced pressure to give the titled compound 12, 513 mg (37%), as a red viscous liquid.

¹H NMR (400 MHz, CDCl₃) δ ppm: 6.90-6.86 (m, 2H), 3.97 (s, 2H), 3.82 (s, 3H), 3.62 (s, 9H).

### Example 9

### Synthesis of methyl ((trihydroxysilyl)methyl fumarate (Compound 13)

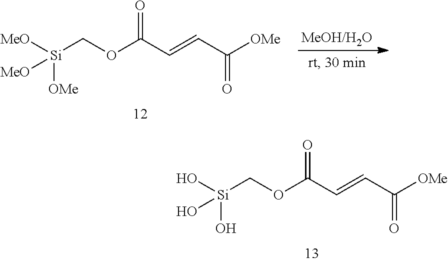 [SMILES: COC(=O)/C=C/C(=O)OC[Si](OC)(OC)OC ; COC(=O)/C=C/C(=O)OC[Si](O)(O)O]

To a solution of 12 (1.0 g, 3.8 mmol, 1.0 equiv., prepared in Example 2) in MeOH (10 mL) at room temperature was added water (341 mg, 19.0 mmol, 5.0 equiv.) dropwise. After addition, the mixture was stirred at room temperature for 30 minutes, with white solids precipitated out. The solids were collected through filtration, washed with methanol three times, and dried at 60° C. in vacuo, to provide the titled compound 13, 500 mg (59%), as a white solid.

¹H NMR (400 MHz, DMSO-d6) δ ppm: 6.79-6.74 (m, 2H), 3.91-3.58 (m, 6H), 3.18-3.15 (m, 2H).

### Example 10

### Synthesis of trimethyl (methylsilanetriyl)trifumarate (Compound 14)

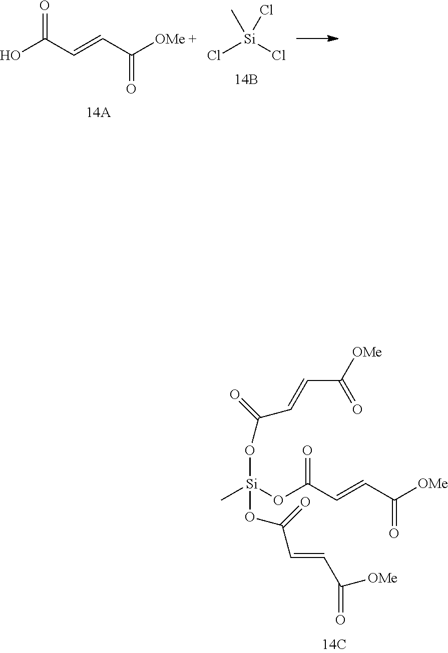

Following the procedure described in Scheme 9, monomethyl fumarate 14A would react with trichloromethane-silane 14B in refluxing toluene or hexanes with a catalytic amount of triethylamine to provide (2′E,2″E)-trimethyl O,O′,O″-(methylsilanetriyl)trifumarate 14C.

### APPENDIX A

| | Common | | |
|---|---|---|---|
| Tissue | Genes | DMF Only | MMF Only |
| Blood | 0 | 6 | 2 |
| Cortex | 1 | 2 | 14 |
| Hippocampus | 1 | 0 | 6 |
| Striatum | 2 | 5 | 12 |
| Jejunum | 51 | 9 | 35 |
| Kidney | 222 | 61 | 94 |
| Liver | 11 | 2 | 34 |
| Spleen | 6 | 2 | 3 |

| | | | | | | | | | |
|---|---|---|---|---|---|---|---|---|---|
| | | | | DMF | DMF | DMF | MMF | MMF | MMF |
| Probe Set | Gene Title | Gene | Entrez | 2 hr | 7 hr | 12 hr | 2 hr | 7 hr | 12 hr |

Blood:

| 1420937_PM_at | cleavage and | Cpsf2 | | −1.65 | | | |
|---|---|---|---|---|---|---|---|
| | polyadenylation | | | | | | |
| | specific factor 2 | | | | | | |
| 1417898_PM_a_at | granzyme A | Gzma | 14938 | | 1.92 | | |
| 1422089_PM_at | natural cytotoxicity | Ncr1 | 17086 | | 1.86 | | |
| | triggering receptor 1 | | | | | | |
| 1425005_PM_at | killer cell lectin-like | Klrc1 | 16641 | | 1.85 | | |
| | receptor subfamily C, | | | | | | |
| | member 1 | | | | | | |
| 1445399_PM_at | killer cell lectin-like | Klrb1b | 80782 | | 1.65 | | |
| | receptor subfamily B | | | | | | |
| | member 1B | | | | | | |
| 1458642_PM_at | killer cell lectin-like | Klre1 | 243655 | | 1.85 | | |
| | receptor family | | | | | | |
| | E member 1 | | | | | | |
| 1418133_PM_at | B-cell leukemia/ | Bcl3 | | | | −1.23 | |
| | lymphoma 3 | | | | | | |
| 1415943_PM_at | syndecan 1 | Sdc1 | | | | | −1.96 |
| | | Common | DMF | MMF | | | |
| | | 0 | 6 | 2 | | | |

Cortex:

| 1419086_PM_at | fibroblast growth | Fgfbp1 | 14181 | | 1.54 | | | 1.62 | |
|---|---|---|---|---|---|---|---|---|---|
| | factor binding | | | | | | | | |
| | protein 1 | | | | | | | | |
| 1448140_PM_at | cytokine induced | Ciapin1 | 109006 | −1.02 | | | | | |
| | apoptosis inhibitor 1 | | | | | | | | |
| 1423627_PM_at | NAD(P)H dehydrogenase, | Nqo1 | 18104 | | 1.50 | | | | |
| | quinone 1 | | | | | | | | |
| 1416734_PM_at | muskelin 1, | Mkln1 | 27418 | | | | −1.85 | | |
| | intracellular mediator | | | | | | | | |
| | containing kelch motifs | | | | | | | | |
| 1420670_PM_at | aryl hydrocarbon | Arnt2 | 11864 | | | | −1.66 | | |
| | receptor nuclear | | | | | | | | |
| | translocator 2 | | | | | | | | |
| 1421166_PM_at | attractin | Atrn | 11990 | | | | −1.62 | | |
| 1421339_PM_at | exostoses (multiple)- | Extl3 | 54616 | | | | −1.57 | | |
| | like 3 | | | | | | | | |
| 1423594_PM_a_at | endothelin receptor | Ednrb | 13618 | | | | −1.53 | | |
| | type B | | | | | | | | |
| 1429607_PM_at | trafficking protein, | Trak2 | 70827 | | | | −1.57 | | |
| | kinesin binding 2 | | | | | | | | |
| 1429992_PM_at | spermatogenesis | Speer4b | 73526 | | | | −1.60 | | |
| | associated glutamate | | | | | | | | |
| | (E)-rich protein 4b | | | | | | | | |
| 1430827_PM_a_at | PTK2 protein tyrosine | Ptk2 | 14083 | | | | −1.53 | | |
| | kinase 2 | | | | | | | | |
| 1438108_PM_at | pleckstrin homology | Plekhm3 | 241075 | | | | −1.50 | | |
| | domain containing, | | | | | | | | |
| | family M, member 3 | | | | | | | | |
| 1442277_PM_at | choline kinase alpha | Chka | 12660 | | | | −1.53 | | |
| 1443123_PM_at | tetratricopeptide | Tanc2 | 77097 | | | | −1.72 | | |
| | repeat, ankyrin | | | | | | | | |
| | repeat and coiled-coil | | | | | | | | |
| | containing 2 | | | | | | | | |
| 1448458_PM_at | topoisomerase (DNA) II | Top2b | 21974 | | | | −1.51 | | |
| | beta | | | | | | | | |
| 1456070_PM_at | protein tyrosine | Ptprg | 19270 | | | | −1.83 | | |
| | phosphatase, receptor | | | | | | | | |
| | type, G | | | | | | | | |
| 1419435_PM_at | aldehyde oxidase 1 | Aox1 | 11761 | | | | | 1.65 | 1.54 |
| | | | | Common | DMF | MMF | | | |
| | | | | 1 | 2 | 14 | | | |

Hippocapmus:

| | | | | | | |
|---|---|---|---|---|---|---|
| 1458305_PM_at | transmembrane and | Tmtc3 | 237500 | −2.38 | | −2.40 |
| | tetratricopeptide | | | | | |
| | repeat containing 3 | | | | | |
| 1417975_PM_at | karyopherin (importin) | Kpna4 | 16649 | | −1.51 | |
| | alpha 4 | | | | | |
| 1425077_PM_at | DnaJ (Hsp40) | Dnajc18 | 76594 | | −1.58 | |
| | homolog, subfamily C, | | | | | |
| | member 18 | | | | | |
| 1426029_PM_a_at | nuclear factor of | Nfat5 | 54446 | | −1.51 | |
| | activated T-cells 5 | | | | | |
| 1434407_PM_at | SLIT-ROBO Rho GTPase | Srgap2 | 14270 | | −1.50 | |
| | activating protein 2 | | | | | |
| 1438085_PM_at | HEAT repeat | Heatr5b | 320473 | | −1.56 | |
| | containing 5B | | | | | |
| 1458421_PM_at | potassium voltage- | Kcnq3 | 110862 | | −1.50 | |
| | gated channel, | | | | | |
| | subfamily Q, | | | | | |
| | member 3 | | | | | |

Striatum:

| 1419435_PM_at | aldehyde oxidase 1 | Aox1 | 11761 | | 1.64 | | | 1.95 | |
|---|---|---|---|---|---|---|---|---|---|
| 1428320_PM_at | KDM3B lysine (K)- | Kdm3b | 277250 | −1.54 | | | −1.56 | | |
| | specific | | | | | | | | |
| | demethylase 3B | | | | | | | | |
| 1434030_PM_at | GTP-binding protein | Gtpbp10 | 207704 | −1.52 | | | | | |
| | 10 (putative) | | | | | | | | |
| 1456070_PM_at | protein tyrosine | Ptprg | 19270 | −1.54 | | | | | |
| | phosphatase, | | | | | | | | |
| | receptor type, G | | | | | | | | |
| 1416734_PM_at | muskelin 1, | Mkln1 | 27418 | −1.97 | | | | | |
| | intracellular | | | | | | | | |
| | mediator containing | | | | | | | | |
| | kelch motifs | | | | | | | | |
| 1438236_PM_at | nuclear factor I/A | Nfia | 18027 | | | −1.53 | | | |
| 1422748_PM_at | zinc finger E-box | Zeb2 | 24136 | | | −1.66 | | | |
| | binding homeobox 2 | | | | | | | | |
| 1427125_PM_s_at | leucine rich repeat | Lrrc41 | 230654 | | | | −1.54 | | |
| | containing 41 | | | | | | | | |
| 1448458_PM_at | topoisomerase | Top2b | 21974 | | | | −1.56 | | |
| | (DNA) II beta | | | | | | | | |
| 1449616_PM_s_at | golgi autoantigen, | Golga3 | 269682 | | | | −1.63 | | |
| | golgin subfamily a, 3 | | | | | | | | |
| 1422556_PM_at | guanine nucleotide | Gna13 | 14674 | | | | −1.63 | | |
| | binding protein, | | | | | | | | |
| | alpha 13 | | | | | | | | |
| 1421616_PM_at | glutamate receptor, | Grin2a | 14811 | | | | −1.78 | | |
| | ionotropic, | | | | | | | | |
| | NMDA2A (epsilon 1) | | | | | | | | |
| 1428676_PM_at | transmembrane | Tmprss6 | 71753 | | | | | | 1.68 |
| | serine protease 6 | | | | | | | | |
| 1448807_PM_at | histamine receptor H3 | Hrh3 | 99296 | | | | | | 1.59 |
| 1437760_PM_at | UDP-N-acetyl- | Galnt12 | 230145 | | | | | | 1.50 |
| | alpha-D-galactosamine: | | | | | | | | |
| | polypeptide | | | | | | | | |
| | N-acetylgalactos- | | | | | | | | |
| | aminyl transferase 12 | | | | | | | | |
| 1435272_PM_at | inositol 1,4,5- | Itpkb | 320404 | | | | | | −1.56 |
| | trisphosphate | | | | | | | | |
| | 3-kinase B | | | | | | | | |
| 1455123_PM_at | suppression of | St18 | 240690 | | | | | | −1.69 |
| | tumorigenicity 18 | | | | | | | | |
| 1457257_PM_x_at | poliovirus receptor- | Pvrl3 | 58998 | | | | | | −1.80 |
| | related 3 | | | | | | | | |
| 1437785_PM_at | a disintegrin-like | Adamts9 | 101401 | | | | | | −2.58 |
| | and metallopeptidase | | | | | | | | |
| | (reprolysin type) | | | | | | | | |
| | with thrombospondin | | | | | | | | |
| | type 1 motif, 9 | | | | | | | | |
| | | | | Common | DMF | MMF | | | |
| | | | | 2 | 5 | 12 | | | |

Jejunum:

| | | | | | | | | | |
|---|---|---|---|---|---|---|---|---|---|
| 1427912_PM_at | carbonyl reductase 3 | Cbr3 | 109857 | 54.72 | 22.39 | 25.95 | 55.49 | 40.98 | 32.24 |
| 1448894_PM_at | aldo-keto | Akr1b8 | 14187 | 7.35 | 8.53 | 17.57 | 8.24 | 14.16 | 18.78 |
| | reductase family | | | | | | | | |
| | 1, member B8 | | | | | | | | |
| 1449486_PM_at | carboxylesterase 1G | Ces1g | 12623 | 4.06 | 4.67 | 7.40 | 3.68 | 9.24 | 9.45 |
| 1423436_PM_at | glutathione | Gsta3 | 14859 | 4.03 | 4.27 | 10.36 | 3.64 | 5.71 | 10.80 |
| | S-transferase, alpha 3 | | | | | | | | |
| 1419622_PM_at | UDP glucuronosyl- | Ugt2b5 | 22238 | 3.40 | 3.42 | 4.92 | 3.13 | 4.50 | 5.00 |
| | transferase 2 | | | | | | | | |
| | family, | | | | | | | | |
| | polypeptide B5 | | | | | | | | |
| 1439624_PM_at | UDP glucuronosyl- | Ugt2b35 | 243085 | 3.22 | 2.66 | 2.37 | 3.08 | 3.31 | 2.45 |
| | transferase 2 | | | | | | | | |
| | family, | | | | | | | | |
| | polypeptide B35 | | | | | | | | |
| 1424266_PM_s_at | carboxylesterase 1F | Ces1f | 234564 | 2.48 | 3.58 | 6.17 | 2.71 | 5.19 | 6.54 |
| 1455454_PM_at | aldo-keto | Akr1c19 | 432720 | 2.45 | 2.07 | 3.20 | 2.33 | 2.39 | 3.53 |
| | reductase family | | | | | | | | |
| | 1, member C19 | | | | | | | | |
| 1427473_PM_at | glutathione | Gstm3 | 14864 | 2.37 | 2.82 | 2.03 | 2.51 | 3.46 | 2.14 |
| | S-transferase, mu 3 | | | | | | | | |
| 1416416_PM_x_at | glutathione | Gstm1 | 14862 | 2.30 | 3.19 | 4.15 | 2.38 | 4.80 | 3.70 |
| | S-transferase, mu 1 | | | | | | | | |
| 1421816_PM_at | glutathione reductase | Gsr | 14782 | 2.23 | 2.83 | 2.20 | 2.19 | 3.13 | 2.67 |
| 1423627_PM_at | NAD(P)H dehydrogenase, | Nqo1 | 18104 | 2.16 | 2.31 | 2.30 | 2.16 | 2.63 | 2.34 |
| | quinone 1 | | | | | | | | |
| 1441931_PM_x_at | glutathione synthetase | Gss | 14854 | 1.95 | 1.97 | 1.53 | 1.92 | 2.20 | 1.55 |
| 1435405_PM_at | SET domain containing 4 | Setd4 | 224440 | 1.91 | 1.90 | 1.66 | 2.11 | 2.27 | 1.60 |
| 1416368_PM_at | glutathione | Gsta4 | 14860 | 1.72 | 2.09 | 2.58 | 1.73 | 2.52 | 2.66 |
| | S-transferase, | | | | | | | | |
| | alpha 4 | | | | | | | | |
| 1421040_PM_a_at | glutathione | Gsta2 | 14858 | 3.82 | 5.81 | 5.65 | | 9.84 | 6.66 |
| | S-transferase, | | | | | | | | |
| | alpha 2 (Yc2) | | | | | | | | |
| 1455595_PM_at | UDP glucuronosyl- | Ugt2b36 | 231396 | 2.07 | 2.23 | 2.94 | | 2.50 | 3.03 |
| | transferase 2 | | | | | | | | |
| | family, polypeptide B36 | | | | | | | | |
| 1419510_PM_at | carboxylesterase 1E | Ces1e | 13897 | 1.85 | 2.10 | 2.70 | | 2.44 | 2.77 |
| 1446542_PM_at | acyl-CoA | Acss2 | 60525 | 1.68 | 1.71 | | 1.84 | 2.06 | 1.52 |
| | synthetase short- | | | | | | | | |
| | chain family member 2 | | | | | | | | |
| 1418672_PM_at | aldo-keto | Akr1c13 | 27384 | 1.62 | 1.61 | 1.96 | | 1.86 | 2.01 |
| | reductase family | | | | | | | | |
| | 1, member C13 | | | | | | | | |
| 1422327_PM_s_at | glucose-6-phosphate | G6pd2 /// | 14380 /// | | 1.82 | 1.74 | 1.74 | 2.06 | 1.83 |
| | dehydrogenase 2 /// | G6pdx | 14381 | | | | | | |
| | glucose-6- | | | | | | | | |
| | phosphate | | | | | | | | |
| | dehydrogenase | | | | | | | | |
| | X-linked | | | | | | | | |
| 1424296_PM_at | glutamate- | Gclc | 14629 | 4.25 | 2.06 | | 3.99 | 2.87 | |
| | cysteine ligase, | | | | | | | | |
| | catalytic subunit | | | | | | | | |
| 1460196_PM_at | carbonyl | Cbr1 | 12408 | 1.55 | 1.65 | | 1.53 | 1.83 | |
| | reductase 1 | | | | | | | | |
| 1424835_PM_at | glutathione | Gstm4 | 14865 | | 3.83 | 5.03 | | 5.75 | 5.43 |
| | S-transferase, mu 4 | | | | | | | | |
| 1429001_PM_at | pirin | Pir | 69656 | | 3.79 | 3.79 | | 5.12 | 4.19 |
| 1422072_PM_a_at | glutathione | Gstm6 | 14867 | | 1.94 | 1.95 | | 2.23 | 2.19 |
| | S-transferase, mu 6 | | | | | | | | |
| 1428988_PM_at | ATP-binding | Abcc3 | 76408 | | 1.88 | 2.02 | | 2.26 | 2.10 |
| | cassette, sub- | | | | | | | | |
| | family C (CFTR/MRP), | | | | | | | | |
| | member 3 | | | | | | | | |
| 1421041_PM_s_at | predicted gene 3776 /// | Gm3776 /// | 100042295 /// | | 1.72 | 2.04 | | 1.90 | 2.09 |
| | glutathione | Gsta1 /// | 14857 /// | | | | | | |
| | S-transferase, | Gsta2 | 14858 | | | | | | |
| | alpha 1 (Ya) /// | | | | | | | | |
| | glutathione | | | | | | | | |
| | S-transferase, | | | | | | | | |
| | alpha 2 (Yc2) | | | | | | | | |
| 1422757_PM_at | solute carrier | Slc5a4b | 64454 | | 1.67 | 2.21 | | 1.79 | 2.16 |
| | family 5 (neutral | | | | | | | | |
| | amino acid | | | | | | | | |
| | transporters, | | | | | | | | |
| | system A), | | | | | | | | |
| | member 4b | | | | | | | | |
| 1437662_PM_at | acyl-CoA synthetase | Acsm5 | 272428 | | 1.60 | 1.86 | | 1.71 | 1.95 |
| | medium-chain | | | | | | | | |
| | family member 5 | | | | | | | | |
| 1416411_PM_at | glutathione | Gstm2 | 14863 | | 1.58 | 2.04 | | 2.09 | 2.20 |
| | S-transferase, mu 2 | | | | | | | | |
| 1441604_PM_at | Esterase D/formyl- | Esd | 13885 | 1.71 | | | 1.52 | 2.15 | |
| | glutathione hydrolase | | | | | | | | |
| 1437240_PM_at | phosphoglucomutase 2 | Pgm2 | 72157 | 2.15 | | | 2.09 | 1.54 | |
| 1421134_PM_at | amphiregulin | Areg | 11839 | 3.58 | | | 2.71 | 2.93 | |
| 1416552_PM_at | developmental | Dppa5a | 434423 | 3.07 | | | 2.64 | 1.88 | |
| | pluripotency | | | | | | | | |
| | associated 5A | | | | | | | | |
| 1418320_PM_at | protease, serine, | Prss8 | 76560 | 2.60 | | | 2.41 | 1.75 | |
| | 8 (prostasin) | | | | | | | | |
| 1451814_PM_a_at | HIV-1 tat interactive | Htatip2 | 53415 | | 1.51 | | 1.53 | 1.68 | |
| | protein 2, homolog | | | | | | | | |
| | (human) | | | | | | | | |
| 1450455_PM_s_at | aldo-keto | Akr1c12 /// | 27384 /// | | | 1.72 | | 1.58 | 1.76 |
| | reductase family | Akr1c13 | 622402 | | | | | | |
| | 1, member C12 /// | | | | | | | | |
| | aldo-keto | | | | | | | | |
| | reductase family | | | | | | | | |
| | 1, member C13 | | | | | | | | |
| 1449279_PM_at | glutathione | Gpx2 | 14776 | | | 1.67 | | 1.53 | 1.54 |
| | peroxidase 2 | | | | | | | | |
| 1418601_PM_at | aldehyde dehydrogenase | Aldh1a7 | 26358 | | | 1.65 | | 1.86 | 1.74 |
| | family 1, subfamily A7 | | | | | | | | |
| 1451386_PM_at | biliverdin reductase B | Blvrb | 233016 | | | 1.53 | 1.57 | 1.55 | 1.54 |
| | (flavin reductase | | | | | | | | |
| | (NADPH)) | | | | | | | | |
| 1419431_PM_at | epiregulin | Ereg | 13874 | 2.50 | | | 2.00 | | |
| 1417061_PM_at | solute carrier | Slc40a1 | 53945 | 2.33 | | | 2.73 | | |
| | family 40 (iron- | | | | | | | | |
| | regulated transporter), | | | | | | | | |
| | member 1 | | | | | | | | |
| 1448916_PM_at | v-maf | Mafg | 17134 | 2.20 | | | 1.95 | | |
| | musculoaponeurotic | | | | | | | | |
| | fibrosarcoma oncogene | | | | | | | | |
| | family, protein | | | | | | | | |
| | G (avian) | | | | | | | | |
| 1424022_PM_at | oxidative stress | Osgin1 | 71839 | 2.08 | | | 1.98 | | |
| | induced growth | | | | | | | | |
| | inhibitor 1 | | | | | | | | |
| 1419030_PM_at | ERO1-like | Ero1l | 50527 | 1.92 | | | 1.90 | | |
| | (S. cerevisiae) | | | | | | | | |
| 1430135_PM_at | deoxyribonuclease | Dnase2a | 13423 | 1.77 | | | 1.79 | | |
| | II alpha | | | | | | | | |
| 1425351_PM_at | sulfiredoxin 1 homolog | Srxn1 | 76650 | 1.71 | | | 1.77 | | |
| | (S. cerevisiae) | | | | | | | | |
| 1436600_PM_at | TOX high mobility group | Tox3 | 244579 | 1.70 | | | 1.81 | | |
| | box family member 3 | | | | | | | | |
| 1433763_PM_at | ectonucleoside | Entpd5 | 12499 | 1.53 | | | | 1.60 | |
| | triphosphate | | | | | | | | |
| | diphosphohydrolase 5 | | | | | | | | |
| 1428805_PM_at | solute carrier | Slc35e3 | 215436 | 1.53 | | | | 1.67 | |
| | family 35, member E3 | | | | | | | | |
| 1426215_PM_at | dopa decarboxylase | Ddc | 13195 | | 1.74 | | | 2.03 | |
| 1424487_PM_x_at | thioredoxin | Txnrd1 | 50493 | | 1.60 | | | 2.07 | |
| | reductase 1 | | | | | | | | |
| 1448354_PM_at | glucose-6-phosphate | G6pdx | 14381 | | 1.54 | | | 1.76 | |
| | dehydrogenase | | | | | | | | |
| | X-linked | | | | | | | | |
| 1440230_PM_at | tsukushin | Tsku | 244152 | 1.61 | | | | | |
| 1453421_PM_at | serine racemase | Srr | 27364 | 1.99 | | | | | |
| 1428834_PM_at | dual specificity | Dusp4 | 319520 | 1.51 | | | | | |
| | phosphatase 4 | | | | | | | | |
| 1429950_PM_at | unc-5 homolog | Unc5c1 | 76589 | −1.53 | | | | | |
| | C (C. elegans)-like | | | | | | | | |
| 1420393_PM_at | nitric oxide synthase 2, | Nos2 | 18126 | −2.39 | | | | | |
| | inducible | | | | | | | | |
| 1442923_PM_at | PTK6 protein | Ptk6 | 20459 | −2.42 | | | | | |
| | tyrosine kinase 6 | | | | | | | | |
| 1426501_PM_a_at | TRAF-interacting | Tifa | 211550 | −2.43 | | | | | |
| | protein with | | | | | | | | |
| | forkhead-associated | | | | | | | | |
| | domain | | | | | | | | |
| 1428397_PM_at | UDP-Gal:betaGlcNAc | B3galt5 | 93961 | −2.77 | | | | | |
| | beta 1,3-galactosyl- | | | | | | | | |
| | transferase, | | | | | | | | |
| | polypeptide 5 | | | | | | | | |
| 1456611_PM_at | family with sequence | Fam13a | 58909 | 1.54 | | | | | |
| | similarity 13, member A | | | | | | | | |
| 1440882_PM_at | low density lipoprotein | Lrp8 | 16975 | | | | 2.23 | | |
| | receptor-related | | | | | | | | |
| | protein 8, | | | | | | | | |
| | apolipoprotein e | | | | | | | | |
| | receptor | | | | | | | | |
| 1448239_PM_at | heme oxygenase | Hmox1 | 15368 | | | | 1.81 | | |
| | (decycling) 1 | | | | | | | | |
| 1436605_PM_at | transketolase | Tkt | 21881 | | | | 1.73 | | |
| 1422645_PM_at | hemochromatosis | Hfe | 15216 | | | | 1.73 | | |
| 1426600_PM_at | solute carrier | Slc2a1 | 20525 | | | | 1.68 | | |
| | family 2 (facilitated | | | | | | | | |
| | glucose transporter), | | | | | | | | |
| | member 1 | | | | | | | | |
| 1438953_PM_at | c-fos induced | Figf | 14205 | | | | 1.63 | | |
| | growth factor | | | | | | | | |
| 1450410_PM_a_at | solute carrier | Slc48a1 | 67739 | | | | 1.62 | | |
| | family 48 (heme | | | | | | | | |
| | transporter), member 1 | | | | | | | | |
| 1452837_PM_at | lipin 2 | Lpin2 | 64898 | | | | 1.61 | | |
| 1424181_PM_at | septin 6 | 6-Sep | 56526 | | | | 1.56 | | |
| 1449078_PM_at | ST3 beta-galactoside | St3gal6 | 54613 | | | | 1.52 | | |
| | alpha-2,3- | | | | | | | | |
| | sialyltransferase 6 | | | | | | | | |
| 1416418_PM_at | gamma-aminobutyric | Gabarapl1 | 57436 | | | | 1.52 | | |
| | acid (GABA) A | | | | | | | | |
| | receptor-associated | | | | | | | | |
| | protein-like 1 | | | | | | | | |
| 1423635_PM_at | bone morphogenetic | Bmp2 | 12156 | | | | −1.55 | | |
| | protein 2 | | | | | | | | |
| 1421886_PM_at | son of sevenless | Sos1 | 20662 | | | | −1.57 | | |
| | homolog 1 | | | | | | | | |
| | (Drosophila) | | | | | | | | |
| 1452834_PM_at | proline rich 5 like | Prr5l | 72446 | | | | −1.64 | | |
| 1433699_PM_at | tumor necrosis | Tnfaip3 | 21929 | | | | −1.67 | | |
| | factor, alpha- | | | | | | | | |
| | induced protein 3 | | | | | | | | |
| 1450165_PM_at | schlafen 2 | Slfn2 | 20556 | | | | −1.82 | | |
| 1416632_PM_at | malic enzyme 1, | Me1 | 17436 | | | | | 2.01 | |
| | NADP(+)-dependent, | | | | | | | | |
| | cytosolic | | | | | | | | |
| 1419072_PM_at | glutathione | Gstm7 | 68312 | | | | | 1.97 | |
| | S-transferase, mu 7 | | | | | | | | |
| 1451385_PM_at | family with sequence | Fam162a | 70186 | | | | | 1.67 | |
| | similarity 162, | | | | | | | | |
| | member A | | | | | | | | |
| 1451095_PM_at | asparagine synthetase | Asns | 27053 | | | | | 1.60 | |
| 1419442_PM_at | matrilin 2 | Matn2 | 17181 | | | | | 1.59 | |
| 1423706_PM_a_at | phosphogluconate | Pgd | 110208 | | | | | 1.55 | |
| | dehydrogenase | | | | | | | | |
| 1450109_PM_s_at | ATP-binding | Abcc2 | 12780 | | | | | 1.51 | |
| | cassette, sub- | | | | | | | | |
| | family C (CFTR/MRP), | | | | | | | | |
| | member 2 | | | | | | | | |
| 1428960_PM_at | enkurin, TRPC channel | Enkur | 71233 | | | | | −1.56 | |
| | interacting protein | | | | | | | | |
| 1418580_PM_at | receptor transporter | Rtp4 | 67775 | | | | | −1.61 | |
| | protein 4 | | | | | | | | |
| 1435529_PM_at | predicted gene 14446 | Gm14446 | 667373 | | | | | −1.65 | |
| 1437960_PM_at | calpain 13 | Capn13 | 381122 | | | | | −1.75 | |
| 1436058_PM_at | radical S-adenosyl | Rsad2 | 58185 | | | | | −1.81 | |
| | methionine domain | | | | | | | | |
| | containing 2 | | | | | | | | |
| 1450783_PM_at | interferon- | Ifit1 | 15957 | | | | | −1.95 | |
| | induced protein with | | | | | | | | |
| | tetratricopeptide | | | | | | | | |
| | repeats 1 | | | | | | | | |
| 1422438_PM_at | epoxide hydrolase 1, | Ephx1 | 13849 | | | | | | 2.17 |
| | microsomal | | | | | | | | |
| 1434458_PM_at | follistatin | Fst | 14313 | | | | | | 1.94 |
| 1422000_PM_at | aldo-keto | Akr1c12 | 622402 | | | | | | 1.52 |
| | reductase family | | | | | | | | |
| | 1, member C12 | | | | | | | | |
| 1417991_PM_at | deiodinase, | Dio1 | 13370 | | | | | | −1.51 |
| | iodothyronine, | | | | | | | | |
| | type I | | | | | | | | |
| 1451642_PM_at | kinesin family | Kif1b | 16561 | | | | | | −1.51 |
| | member 1B | | | | | | | | |
| 1457554_PM_at | apolipoprotein B | Apob | 238055 | | | | | | −1.54 |

Kidney:

| | | | | | | | | | |
|---|---|---|---|---|---|---|---|---|---|
| 1418949_PM_at | growth | Gdf15 | 23886 | 5.39 | 1.75 | 1.77 | 5.50 | 1.98 | 2.01 |
| | differentiation | | | | | | | | |
| | factor 15 | | | | | | | | |
| 1427912_PM_at | carbonyl reductase 3 | Cbr3 | 109857 | 3.04 | 5.88 | 5.67 | 2.96 | 9.54 | 7.02 |
| 1451095_PM_at | asparagine synthetase | Asns | 27053 | 3.03 | 1.92 | 2.65 | 2.46 | 2.13 | 2.49 |
| 1419253_PM_at | methylenetetrahydro- | Mthfd2 | 17768 | 2.06 | 1.65 | 1.60 | 2.03 | 1.89 | 1.65 |
| | folate | | | | | | | | |
| | dehydrogenase | | | | | | | | |
| | (NAD+ dependent), | | | | | | | | |
| | methenyltetrahydro- | | | | | | | | |
| | folate | | | | | | | | |
| | cyclohydrolase | | | | | | | | |
| 1423436_PM_at | glutathione | Gsta3 | 14859 | 2.01 | 2.14 | 2.66 | 1.93 | 2.95 | 2.85 |
| | S-transferase, | | | | | | | | |
| | alpha 3 | | | | | | | | |
| 1424296_PM_at | glutamate- | Gclc | 14629 | 1.70 | 1.63 | 1.60 | 1.66 | 1.87 | 1.64 |
| | cysteine ligase, | | | | | | | | |
| | catalytic subunit | | | | | | | | |
| 1419942_PM_at | Sulfiredoxin 1 | Srxn1 | 76650 | 1.62 | 2.01 | 1.92 | 1.57 | 2.92 | 2.09 |
| | homolog | | | | | | | | |
| | (S. cerevisiae) | | | | | | | | |
| 1423706_PM_a_at | phosphogluconate | Pgd | 110208 | 1.61 | 1.91 | 2.20 | 1.51 | 2.21 | 2.27 |
| | dehydrogenase | | | | | | | | |
| 1455454_PM_at | aldo-keto | Akr1c19 | 432720 | 1.53 | 1.64 | 1.92 | 1.56 | 1.97 | 2.00 |
| | reductase family | | | | | | | | |
| | 1, member C19 | | | | | | | | |
| 1419435_PM_at | aldehyde oxidase 1 | Aox1 | 11761 | 2.46 | 1.95 | | 2.31 | 2.50 | 2.23 |
| 1421529_PM_a_at | thioredoxin reductase 1 | Txnrd1 | 50493 | 1.53 | 2.82 | 2.31 | | 3.08 | 1.74 |
| 1429813_PM_at | pantothenate kinase 1 | Pank1 | 75735 | −1.59 | −1.64 | −1.53 | | −1.79 | −1.73 |
| 1443797_PM_at | alkylglycerone | Agps | 228061 | −1.62 | | −1.55 | −1.65 | −1.68 | −1.80 |
| | phosphate synthase | | | | | | | | |
| 1441042_PM_at | fibroblast | Fgf1 | 14164 | −2.91 | −1.82 | | −2.48 | −1.71 | −1.58 |
| | growth factor 1 | | | | | | | | |
| 1448239_PM_at | heme oxygenase | Hmox1 | 15368 | 6.27 | 5.61 | | 5.43 | 7.48 | |
| | (decycling) 1 | | | | | | | | |
| 1451612_PM_at | metallothionein 1 | Mt1 | 17748 | 2.79 | 1.66 | | 2.47 | 1.89 | |
| 1418571_PM_at | tumor necrosis | Tnfrsf12a | 27279 | 2.37 | 1.94 | | 2.32 | 2.40 | |
| | factor receptor | | | | | | | | |
| | superfamily, member 12a | | | | | | | | |
| 1438824_PM_at | solute carrier | Slc20a1 | 20515 | 1.89 | 1.55 | | 2.14 | 1.70 | |
| | family 20, member 1 | | | | | | | | |
| 1426645_PM_at | heat shock | Hsp90aa1 | 15519 | 1.64 | | 1.57 | 1.53 | 1.50 | |
| | protein 90, alpha | | | | | | | | |
| | (cytosolic), class | | | | | | | | |
| | A member 1 | | | | | | | | |
| 1435311_PM_s_at | synapsin III | Syn3 | 27204 | −1.73 | −1.67 | | −2.07 | −1.68 | |
| 1445089_PM_at | DNA segment, | D16Ertd778e | 52714 | −2.60 | | | −2.41 | −1.87 | −2.25 |
| | Chr 16, ERATO | | | | | | | | |
| | Doi 778, expressed | | | | | | | | |
| 1430744_PM_at | napsin A aspartic | Napsa | 16541 | −4.53 | −1.60 | | −4.07 | −1.79 | |
| | peptidase | | | | | | | | |
| 1429001_PM_at | pirin | Pir | 69656 | | 2.35 | 2.89 | | 2.63 | 3.08 |
| 1426300_PM_at | activated leukocyte cell | Alcam | 11658 | | 2.08 | 2.05 | | 2.66 | 2.83 |
| | adhesion molecule | | | | | | | | |
| 1423186_PM_at | T-cell lymphoma | Tiam2 | 24001 | | 1.84 | 1.56 | | 2.27 | 1.84 |
| | invasion and | | | | | | | | |
| | metastasis 2 | | | | | | | | |
| 1443870_PM_at | ATP-binding | Abcc4 | 239273 | | 1.78 | 1.81 | | 2.09 | 2.07 |
| | cassette, sub- | | | | | | | | |
| | family C (CFTR/MRP), | | | | | | | | |
| | member 4 | | | | | | | | |
| 1423627_PM_at | NAD(P)H dehydrogenase, | Nqo1 | 18104 | | 1.71 | 2.06 | | 1.96 | 2.11 |
| | quinone 1 | | | | | | | | |
| 1430135_PM_at | deoxyribonuclease | Dnase2a | 13423 | | 1.69 | 2.12 | | 2.20 | 2.15 |
| | II alpha | | | | | | | | |
| 1442291_PM_at | lysophosphatidic | Lpar2 | 53978 | | 1.66 | 1.61 | | 1.84 | 1.93 |
| | acid receptor 2 | | | | | | | | |
| 1421209_PM_s_at | inhibitor of kappaB | Ikbkg | 16151 | | 1.64 | 1.50 | | 1.77 | 1.57 |
| | kinase gamma | | | | | | | | |
| 1448894_PM_at | aldo-keto | Akr1b8 | 14187 | | 1.62 | 1.81 | | 1.86 | 1.68 |
| | reductase family | | | | | | | | |
| | 1, member B8 | | | | | | | | |
| 1422327_PM_s_at | glucose-6-phosphate | G6pd2 /// | 14380 /// | | 1.61 | 1.77 | | 1.86 | 1.91 |
| | dehydrogenase 2 /// | G6pdx | 14381 | | | | | | |
| | glucose-6-phosphate | | | | | | | | |
| | dehydrogenase X-linked | | | | | | | | |
| 1451386_PM_at | biliverdin reductase B | Blvrb | 233016 | | 1.55 | 1.94 | | 1.83 | 2.23 |
| | (flavin reductase | | | | | | | | |
| | (NADPH)) | | | | | | | | |
| 1416368_PM_at | glutathione | Gsta4 | 14860 | | 1.51 | 1.89 | | 1.85 | 1.95 |
| | S-transferase, alpha 4 | | | | | | | | |
| 1448354_PM_at | glucose-6-phosphate | G6pdx | 14381 | | 1.50 | 1.55 | | 1.75 | 1.70 |
| | dehydrogenase | | | | | | | | |
| | X-linked | | | | | | | | |
| 1430111_PM_a_at | branched chain | Bcat1 | 12035 | | −1.56 | −1.72 | | −1.59 | −2.07 |
| | aminotransferase | | | | | | | | |
| | 1, cytosolic | | | | | | | | |
| 1452733_PM_at | pantothenate kinase 2 | Pank2 | 74450 | | −1.77 | −1.65 | | −1.94 | −1.97 |
| 1418358_PM_at | sperm mitochondria- | Smcp | 17235 | | −1.84 | −1.87 | | −1.96 | −2.49 |
| | associated cysteine-rich | | | | | | | | |
| | protein | | | | | | | | |
| 1436101_PM_at | ring finger protein 24 | Rnf24 | 51902 | | −1.88 | −1.68 | | −2.03 | −2.01 |
| 1437199_PM_at | dual specificity | Dusp5 | 240672 | 3.68 | | | 4.07 | 1.74 | |
| | phosphatase 5 | | | | | | | | |
| 1419086_PM_at | fibroblast growth factor | Fgfbp1 | 14181 | 1.81 | | | 1.94 | 1.73 | |
| | binding protein 1 | | | | | | | | |
| 1448566_PM_at | solute carrier | Slc40a1 | 53945 | 1.71 | | | 1.57 | 1.51 | |
| | family 40 (iron- | | | | | | | | |
| | regulated transporter), | | | | | | | | |
| | member 1 | | | | | | | | |
| 1450957_PM_a_at | sequestosome 1 | Sqstm1 | 18412 | 1.69 | | | 1.67 | 1.62 | |
| 1418627_PM_at | glutamate- | Gclm | 14630 | 1.51 | | 1.50 | | 1.64 | |
| | cysteine ligase, | | | | | | | | |
| | modifier subunit | | | | | | | | |
| 1435005_PM_at | centromere | Cenpe | 229841 | −2.20 | | | −2.08 | −1.60 | |
| | protein E | | | | | | | | |
| 1428223_PM_at | major facilitator | Mfsd2a | 76574 | −2.59 | −3.07 | | | −3.38 | |
| | superfamily domain | | | | | | | | |
| | containing 2A | | | | | | | | |
| 1421398_PM_at | tripartite motif- | Trim7 | 94089 | −3.34 | | | −2.74 | −1.66 | |
| | containing 7 | | | | | | | | |
| 1444138_PM_at | cytochrome | Cyp2r1 | 244209 | | 1.74 | | | 1.67 | 1.58 |
| | P450, family 2, | | | | | | | | |
| | subfamily r, | | | | | | | | |
| | polypeptide 1 | | | | | | | | |
| 1415983_PM_at | lymphocyte | Lcp1 | 18826 | | 1.53 | | | 1.59 | 1.54 |
| | cytosolic protein 1 | | | | | | | | |
| 1434716_PM_at | hepatitis A virus | Havcr1 | 171283 | | | 4.46 | | 3.22 | 4.79 |
| | cellular receptor 1 | | | | | | | | |
| 1440330_PM_at | Histone cluster | Hist1h2be | 319179 | | | 3.25 | | 1.98 | 2.92 |
| | 1, H2be | | | | | | | | |
| 1418601_PM_at | aldehyde dehydrogenase | Aldh1a7 | 26358 | | | 2.48 | | 2.16 | 3.02 |
| | family 1, subfamily A7 | | | | | | | | |
| 1424126_PM_at | aminolevulinic | Alas1 | 11655 | | | 1.88 | | 1.51 | 2.02 |
| | acid synthase 1 | | | | | | | | |
| 1449937_PM_at | endonuclease, | Endou | 19011 | | | 1.75 | | 1.52 | 1.89 |
| | polyU-specific | | | | | | | | |
| 1442145_PM_at | ATPase type 13A3 | Atp13a3 | 224088 | | | 1.64 | | 1.52 | 1.62 |
| 1450298_PM_at | tumor necrosis | Tnfsf14 | 50930 | | | 1.62 | | 1.50 | 1.63 |
| | factor (ligand) | | | | | | | | |
| | superfamily, | | | | | | | | |
| | member 14 | | | | | | | | |
| 1450702_PM_at | hemochromatosis | Hfe | 15216 | | | 1.60 | | 1.57 | 1.56 |
| 1448766_PM_at | gap junction | Gjb1 | 14618 | | | 1.59 | | 1.50 | 1.81 |
| | protein, beta 1 | | | | | | | | |
| 1435663_PM_at | estrogen receptor 1 | Esr1 | 13982 | | | −1.54 | | −1.54 | −1.75 |
| | (alpha) | | | | | | | | |
| 1452975_PM_at | alanine-glyoxylate | Agxt2l1 | 71760 | | | −1.65 | −1.88 | −1.74 | |
| | aminotransferase | | | | | | | | |
| | 2-like 1 | | | | | | | | |
| 1454865_PM_at | solute carrier family 9 | Slc9a8 | 77031 | | | −1.98 | | −1.68 | −2.40 |
| | (sodium/hydrogen | | | | | | | | |
| | exchanger), member 8 | | | | | | | | |
| 1422533_PM_at | cytochrome | Cyp51 | 13121 | | | −2.05 | | −1.54 | −1.95 |
| | P450, family 51 | | | | | | | | |
| 1451382_PM_at | ChaC, cation transport | Chac1 | 69065 | 31.00 | | | 30.40 | | |
| | regulator-like 1 | | | | | | | | |
| | (E. coli) | | | | | | | | |
| 1449363_PM_at | activating transcription | Atf3 | 11910 | 5.86 | | | 6.56 | | |
| | factor 3 | | | | | | | | |
| 1424022_PM_at | oxidative stress | Osgin1 | 71839 | 4.63 | | | 3.97 | | |
| | induced growth | | | | | | | | |
| | inhibitor 1 | | | | | | | | |
| 1417065_PM_at | early growth response 1 | Egr1 | 13653 | 4.60 | | | 5.51 | | |
| 1416756_PM_at | DnaJ (Hsp40) homolog, | Dnajb1 | 81489 | 4.36 | | | 3.73 | | |
| | subfamily B, member 1 | | | | | | | | |
| 1429863_PM_at | LON peptidase N-terminal | Lonrf3 | 74365 | 4.13 | | | 3.58 | | |
| | domain and ring finger 3 | | | | | | | | |
| 1417516_PM_at | DNA-damage inducible | Ddit3 | 13198 | 3.97 | | | 4.07 | | |
| | transcript 3 | | | | | | | | |
| 1427126_PM_at | heat shock protein 1B | Hspa1b | 15511 | 3.65 | | | 3.25 | | |
| 1434496_PM_at | polo-like kinase | Plk3 | 12795 | 3.14 | | | 3.55 | | |
| | 3 (Drosophila) | | | | | | | | |
| 1418936_PM_at | v-maf musculoaponeurotic | Maff | 17133 | 2.83 | | | 3.80 | | |
| | fibrosarcoma oncogene | | | | | | | | |
| | family, protein F | | | | | | | | |
| | (avian) | | | | | | | | |
| 1428694_PM_at | MIR17 host gene 1 (non- | Mir17hg | 75957 | 2.82 | | | 2.23 | | |
| | protein coding) | | | | | | | | |
| 1417930_PM_at | Ngfi-A binding protein 2 | Nab2 | 17937 | 2.74 | | | 2.33 | | |
| 1425305_PM_at | zinc finger protein 295 | Zfp295 | 114565 | 2.68 | | | 2.46 | | |
| 1438133_PM_a_at | cysteine rich protein 61 | Cyr61 | 16007 | 2.64 | | | 2.19 | | |
| 1431182_PM_at | heat shock protein 8 /// | Hspa8 /// | 15481 /// | 2.63 | | | 2.43 | | |
| | hypothetical LOC624853 | LOC624853 | 624853 | | | | | | |
| 1447930_PM_at | bromodomain | Baz1a /// | 100505185 /// | 2.56 | | | 2.24 | | |
| | adjacent to zinc | LOC100505185 | 217578 | | | | | | |
| | finger domain 1A /// | | | | | | | | |
| | bromodomain | | | | | | | | |
| | adjacent to zinc | | | | | | | | |
| | finger domain | | | | | | | | |
| | protein 1A-like | | | | | | | | |
| 1449311_PM_at | BTB and CNC homology 1 | Bach1 | 12013 | 2.55 | | | 2.26 | | |
| 1428963_PM_at | RWD domain containing 2A | Rwdd2a | 69519 | 2.54 | | | 2.10 | | |
| 1434901_PM_at | zinc finger and | Zbtb2 | 381990 | 2.52 | | | 2.39 | | |
| | BTB domain | | | | | | | | |
| | containing 2 | | | | | | | | |
| 1433599_PM_at | bromodomain | Baz1a | 217578 | 2.51 | | | 2.12 | | |
| | adjacent to zinc | | | | | | | | |
| | finger domain 1A | | | | | | | | |
| 1451177_PM_at | DnaJ (Hsp40) homolog, | Dnajb4 | 67035 | 2.48 | | | 2.32 | | |
| | subfamily B, member 4 | | | | | | | | |
| 1421262_PM_at | lipase, endothelial | Lipg | 16891 | 2.42 | | | 2.25 | | |
| 1423566_PM_a_at | heat shock | Hsph1 | 15505 | 2.40 | | | 2.22 | | |
| | 105 kDa/110 kDa | | | | | | | | |
| | protein 1 | | | | | | | | |
| 1452388_PM_at | heat shock protein 1A | Hspa1a | 193740 | 2.35 | | | 2.41 | | |
| 1418932_PM_at | nuclear factor, | Nfil3 | 18030 | 2.29 | | | 2.29 | | |
| | interleukin 3, | | | | | | | | |
| | regulated | | | | | | | | |
| 1424974_PM_at | zinc finger protein 418 | Zfp418 | 232854 | 2.24 | | | 2.08 | | |
| 1448135_PM_at | activating transcription | Atf4 | 11911 | 2.12 | | | 1.94 | | |
| | factor 4 | | | | | | | | |
| 1428834_PM_at | dual specificity | Dusp4 | 319520 | 2.09 | | | 1.93 | | |
| | phosphatase 4 | | | | | | | | |
| 1420056_PM_s_at | jumonji domain | Jmjd6 | 107817 | 2.08 | | | 2.00 | | |
| | containing 6 | | | | | | | | |
| 1418334_PM_at | DBF4 homolog | Dbf4 | 27214 | 2.08 | | | 2.04 | | |
| | (S. cerevisiae) | | | | | | | | |
| 1439094_PM_at | clathrin, heavy | Cltc | 67300 | 2.07 | | | 1.89 | | |
| | polypeptide (Hc) | | | | | | | | |
| 1437210_PM_a_at | bromodomain containing 2 | Brd2 | 14312 | 2.07 | | | 1.87 | | |
| 1417406_PM_at | SERTA domain | Sertad1 | 55942 | 2.06 | | | 1.92 | | |
| | containing 1 | | | | | | | | |
| 1426722_PM_at | solute carrier | Slc38a2 | 67760 | 2.05 | | | 1.78 | | |
| | family 38, member 2 | | | | | | | | |
| 1418591_PM_at | DnaJ (Hsp40) homolog, | Dnaja4 | 58233 | 2.05 | | | 1.87 | | |
| | subfamily A, member 4 | | | | | | | | |
| 1418349_PM_at | heparin-binding | Hbegf | 15200 | 2.03 | | | 2.27 | | |
| | EGF-like growth factor | | | | | | | | |
| 1422452_PM_at | BCL2-associated | Bag3 | 29810 | 2.03 | | | 1.86 | | |
| | athanogene 3 | | | | | | | | |
| 1438725_PM_at | mediator | Med13 | 327987 | 2.02 | | | 1.83 | | |
| | complex subunit 13 | | | | | | | | |
| 1427375_PM_at | RNA (guanine-9-) | Rg9mtd2 | 108943 | 1.98 | | | 1.67 | | |
| | methyltransferase | | | | | | | | |
| | domain | | | | | | | | |
| | containing 2 | | | | | | | | |
| 1453137_PM_at | F-box protein 30 | Fbxo30 | 71865 | 1.97 | | | 1.94 | | |
| 1455665_PM_at | LON peptidase | Lonrf1 | 244421 | 1.96 | | | 1.98 | | |
| | N-terminal | | | | | | | | |
| | domain and ring | | | | | | | | |
| | finger 1 | | | | | | | | |
| 1425185_PM_at | PPPDE peptidase | Pppde1 | 78825 | 1.94 | | | 1.94 | | |
| | domain | | | | | | | | |
| | containing 1 | | | | | | | | |
| 1456909_PM_at | glucose-6-phosphate | LOC676974 | 676974 | 1.94 | | | 1.57 | | |
| | isomerase-like | | | | | | | | |
| 1437884_PM_at | ADP-ribosylation | Arl5b | 75869 | 1.93 | | | 1.69 | | |
| | factor-like 5B | | | | | | | | |
| 1418370_PM_at | troponin C, cardiac/slow | Tnnc1 | 21924 | 1.89 | | | 1.94 | | |
| | skeletal | | | | | | | | |
| 1438764_PM_at | annexin A7 | Anxa7 | 11750 | 1.88 | | | 1.76 | | |
| 1450716_PM_at | a disintegrin-like and | Adamts1 | 11504 | 1.86 | | | 1.92 | | |
| | metallopeptidase | | | | | | | | |
| | (reprolysin type) with | | | | | | | | |
| | thrombospondin | | | | | | | | |
| | type 1 motif, 1 | | | | | | | | |
| 1429350_PM_at | EP300 interacting | Eid3 | 66341 | 1.84 | | | 1.64 | | |
| | inhibitor of | | | | | | | | |
| | differentiation 3 | | | | | | | | |
| 1431140_PM_at | thioesterase | Them4 | 75778 | 1.80 | | | 1.62 | | |
| | superfamily | | | | | | | | |
| | member 4 | | | | | | | | |
| 1418966_PM_a_at | discoidin, CUB | Dcbld1 | 66686 | 1.78 | | | 1.76 | | |
| | and LCCL domain | | | | | | | | |
| | containing 1 | | | | | | | | |
| 1420990_PM_at | chromodomain | Chd1 | 12648 | 1.76 | | | 1.53 | | |
| | helicase DNA | | | | | | | | |
| | binding protein 1 | | | | | | | | |
| 1416442_PM_at | immediate early | Ier2 | 15936 | 1.75 | | | 1.86 | | |
| | response 2 | | | | | | | | |
| 1449519_PM_at | growth arrest and DNA- | Gadd45a | 13197 | 1.75 | | | 1.76 | | |
| | damage-inducible 45 | | | | | | | | |
| | alpha | | | | | | | | |
| 1426381_PM_at | peroxisome proliferative | Pprc1 | 226169 | 1.74 | | | 1.68 | | |
| | activated receptor, | | | | | | | | |
| | gamma, coactivator- | | | | | | | | |
| | related 1 | | | | | | | | |
| 1428997_PM_at | PHD finger protein 23 | Phf23 | 78246 | 1.72 | | | 1.56 | | |
| 1429056_PM_at | N(alpha)- | Naa16 | 66897 | 1.71 | | | 1.56 | | |
| | acetyltransferase | | | | | | | | |
| | 16, NatA | | | | | | | | |
| | auxiliary subunit | | | | | | | | |
| 1440867_PM_at | sprouty homolog | Spry4 | 24066 | 1.71 | | | 1.85 | | |
| | 4 (Drosophila) | | | | | | | | |
| 1450724_PM_at | family with sequence | Fam126a | 84652 | 1.70 | | | 1.55 | | |
| | similarity 126, member A | | | | | | | | |
| 1437410_PM_at | aldehyde dehydrogenase | Aldh2 | 11669 | 1.69 | | | 1.75 | | |
| | 2, mitochondrial | | | | | | | | |
| 1416288_PM_at | DnaJ (Hsp40) homolog, | Dnaja1 | 15502 | 1.69 | | | 1.59 | | |
| | subfamily A, member 1 | | | | | | | | |
| 1418640_PM_at | sirtuin 1 (silent | Sirt1 | 93759 | 1.68 | | | 1.53 | | |
| | mating type information | | | | | | | | |
| | regulation 2, homolog) 1 | | | | | | | | |
| | (S. cerevisiae) | | | | | | | | |
| 1438842_PM_at | mitochondrial | Mtch2 | 56428 | 1.67 | | | 1.51 | | |
| | carrier homolog | | | | | | | | |
| | 2 (C. elegans) | | | | | | | | |
| 1439093_PM_at | heat shock | Hspa41 | 18415 | 1.67 | | | 1.60 | | |
| | protein 4 like | | | | | | | | |
| 1455658_PM_at | CGG triplet | Cggbp1 | 106143 | 1.67 | | | 1.51 | | |
| | repeat binding | | | | | | | | |
| | protein 1 | | | | | | | | |
| 1433398_PM_at | FYVE, RhoGEF | Fgd3 | 30938 | 1.66 | | | 1.60 | | |
| | and PH domain | | | | | | | | |
| | containing 3 | | | | | | | | |
| 1460511_PM_at | plakophilin 2 | Pkp2 | 67451 | 1.66 | | | 1.67 | | |
| 1451463_PM_at | proline rich 5 (renal) | Prr5 | 109270 | 1.65 | | | 1.86 | | |
| 1435160_PM_at | AHA1, activator | Ahsa2 | 268390 | 1.64 | | | 1.64 | | |
| | of heat shock | | | | | | | | |
| | protein ATPase | | | | | | | | |
| | homolog 2 (yeast) | | | | | | | | |
| 1434967_PM_at | zinc finger, SWIM domain | Zswim6 | 67263 | 1.63 | | | 1.52 | | |
| | containing 6 | | | | | | | | |
| 1416084_PM_at | zinc finger, AN1-type | Zfand5 | 22682 | 1.63 | | | 1.70 | | |
| | domain 5 | | | | | | | | |
| 1423481_PM_at | RIO kinase 2 (yeast) | Riok2 | 67045 | 1.62 | | | 1.58 | | |
| 1433674_PM_a_at | small nucleolar | Snhg1 | 83673 | 1.62 | | | 1.58 | | |
| | RNA host gene | | | | | | | | |
| | (non-protein | | | | | | | | |
| | coding) 1 | | | | | | | | |
| 1441604_PM_at | Esterase D/ | Esd | 13885 | 1.62 | | | 1.57 | | |
| | formylglutathione | | | | | | | | |
| | hydrolase | | | | | | | | |
| 1422054_PM_a_at | SKI-like | Skil | 20482 | 1.62 | | | 1.64 | | |
| 1416042_PM_s_at | nuclear autoantigenic | Nasp | 50927 | 1.62 | | | 1.52 | | |
| | sperm protein | | | | | | | | |
| | (histone-binding) | | | | | | | | |
| 1452239_PM_at | gene trap ROSA | Gt(ROSA)26Sor | 14910 | 1.62 | | | 1.51 | | |
| | 26, Philippe | | | | | | | | |
| | Soriano | | | | | | | | |
| 1454826_PM_at | zinc finger and | Zbtb11 | 271377 | 1.60 | | | 1.53 | | |
| | BTB domain | | | | | | | | |
| | containing 11 | | | | | | | | |
| 1438130_PM_at | TAF15 RNA | Taf15 | 70439 | 1.59 | | | 1.71 | | |
| | polymerase II, | | | | | | | | |
| | TATA box | | | | | | | | |
| | binding protein | | | | | | | | |
| | (TBP)- | | | | | | | | |
| | associated factor | | | | | | | | |
| 1435632_PM_at | nuclear fragile X | Nufip2 | 68564 | 1.57 | | | 1.52 | | |
| | mental retardation | | | | | | | | |
| | protein interacting | | | | | | | | |
| | protein 2 | | | | | | | | |
| 1440255_PM_at | histone H4 | Hinfp | 102423 | 1.57 | | | 1.68 | | |
| | transcription factor | | | | | | | | |
| 1455175_PM_at | PHD finger protein 13 | Phf13 | 230936 | 1.56 | | | 1.51 | | |
| 1416077_PM_at | adrenomedullin | Adm | 11535 | 1.56 | | | 1.59 | | |
| 1415940_PM_at | zinc finger, AN1-type | Zfand2a | 100494 | 1.55 | | | 1.51 | | |
| | domain 2A | | | | | | | | |
| 1423862_PM_at | pleckstrin homology | Plekhf2 | 71801 | 1.54 | | | 1.50 | | |
| | domain containing, | | | | | | | | |
| | family F (with | | | | | | | | |
| | FYVE domain) member 2 | | | | | | | | |
| 1451083_PM_s_at | alanyl-tRNA | Aars | 234734 | 1.53 | | | 1.53 | | |
| | synthetase | | | | | | | | |
| 1434660_PM_at | alkB, alkylation | Alkbh1 | 211064 | 1.53 | | | 1.60 | | |
| | repair homolog | | | | | | | | |
| | 1 (E. coli) | | | | | | | | |
| 1416067_PM_at | interferon-related | Ifrd1 | 15982 | 1.52 | | | 1.53 | | |
| | developmental | | | | | | | | |
| | regulator 1 | | | | | | | | |
| 1420342_PM_at | ganglioside-induced | Gdap10 | 14546 | 1.52 | | | 1.63 | | |
| | differentiation- | | | | | | | | |
| | associated-protein 10 | | | | | | | | |
| 1415834_PM_at | dual specificity | Dusp6 | 67603 | 1.52 | | | 1.57 | | |
| | phosphatase 6 | | | | | | | | |
| 1416600_PM_a_at | regulator of | Rcan1 | 54720 | 1.51 | | | 1.51 | | |
| | calcineurin 1 | | | | | | | | |
| 1458452_PM_at | Ankyrin repeat | Ankrd11 | 77087 | 1.51 | | | 1.56 | | |
| | domain 11 | | | | | | | | |
| 1418401_PM_a_at | dual specificity | Dusp16 | 70686 | 1.50 | | | 1.50 | | |
| | phosphatase 16 | | | | | | | | |
| 1448694_PM_at | Jun oncogene | Jun | 16476 | 1.50 | | | 1.58 | | |
| 1417639_PM_at | solute carrier | Slc22a4 | 30805 | −1.51 | | | −1.52 | | |
| | family 22 | | | | | | | | |
| | (organic cation | | | | | | | | |
| | transporter), | | | | | | | | |
| | member 4 | | | | | | | | |
| 1439680_PM_at | tumor necrosis | Tnfsf10 | 22035 | −1.52 | | | −1.60 | | |
| | factor (ligand) | | | | | | | | |
| | superfamily, | | | | | | | | |
| | member 10 | | | | | | | | |
| 1450184_PM_s_at | thyrotroph embryonic | Tef | 21685 | −1.56 | | | −1.61 | | |
| | factor | | | | | | | | |
| 1447818_PM_x_at | Ras homolog | Rhebl1 | 69159 | −1.57 | | | −1.74 | | |
| | enriched in brain | | | | | | | | |
| | like 1 | | | | | | | | |
| 1458503_PM_at | B-cell | Bcl7a | 77045 | −1.59 | | | −1.54 | | |
| | CLL/lymphoma 7A | | | | | | | | |
| 1442436_PM_at | fructosamine 3 kinase | Fn3k | 63828 | −1.60 | | | −1.73 | | |
| 1441988_PM_at | protein phosphatase 1K | Ppm1k | 243382 | −1.62 | | | −1.72 | | |
| | (PP2C domain | | | | | | | | |
| | containing) | | | | | | | | |
| 1456922_PM_at | sorting nexin 29 | Snx29 | 74478 | −1.64 | | | −1.74 | | |
| 1452623_PM_at | zinc finger | Zfp759 | 268670 | −1.65 | | | −1.67 | | |
| | protein 759 | | | | | | | | |
| 1434583_PM_at | transmembrane | Tmem26 | 327766 | −1.66 | | | −1.51 | | |
| | protein 26 | | | | | | | | |
| 1450512_PM_at | netrin 4 | Ntn4 | 57764 | −1.67 | | | −1.57 | | |
| 1424988_PM_at | myosin regulatory light | Mylip | 218203 | −1.70 | | | −1.67 | | |
| | chain interacting | | | | | | | | |
| | protein | | | | | | | | |
| 1415997_PM_at | thioredoxin interacting | Txnip | 56338 | −1.73 | | | −1.71 | | |
| | protein | | | | | | | | |
| 1440765_PM_at | Fraser syndrome | Fras1 | 231470 | −1.73 | | | −1.76 | | |
| | 1 homolog (human) | | | | | | | | |
| 1418955_PM_at | zinc finger protein 93 | Zfp93 | 22755 | −1.74 | | | −1.62 | | |
| 1421224_PM_a_at | HNF1 homeobox B | Hnf1b | 21410 | −1.77 | | | −1.59 | | |
| 1431254_PM_at | kelch repeat and | Kbtbd11 | 74901 | −1.77 | | | −1.52 | | |
| | BTB (POZ) domain | | | | | | | | |
| | containing 11 | | | | | | | | |
| 1433699_PM_at | tumor necrosis | Tnfaip3 | 21929 | −1.83 | | | −1.74 | | |
| | factor, alpha- | | | | | | | | |
| | induced protein 3 | | | | | | | | |
| 1445485_PM_at | DNA segment, | D7Ertd187e | 52196 | −1.83 | | | −1.70 | | |
| | Chr 7, ERATO | | | | | | | | |
| | Doi 187, | | | | | | | | |
| | expressed | | | | | | | | |
| 1430593_PM_at | coiled-coil domain | Ccdc30 | 73332 | −2.01 | | | −1.78 | | |
| | containing 30 | | | | | | | | |
| 1436974_PM_at | transmembrane | Tmem88b | 320587 | −2.24 | | | −2.68 | | |
| | protein 88B | | | | | | | | |
| 1459132_PM_at | basonuclin 2 | Bnc2 | 242509 | −2.24 | | | −2.38 | | |
| 1457770_PM_at | solute carrier | Slc39a14 | 213053 | −2.52 | | | −2.26 | | |
| | family 39 (zinc | | | | | | | | |
| | transporter), | | | | | | | | |
| | member 14 | | | | | | | | |
| 1431740_PM_at | solute carrier family 7, | Slc7a13 | 74087 | −2.60 | | | −2.14 | | |
| | (cationic amino | | | | | | | | |
| | acid transporter, | | | | | | | | |
| | y+ system) member 13 | | | | | | | | |
| 1418918_PM_at | insulin-like | Igfbp1 | 16006 | −2.89 | | | −2.27 | | |
| | growth factor | | | | | | | | |
| | binding protein 1 | | | | | | | | |
| 1449545_PM_at | fibroblast | Fgf18 | 14172 | | 2.44 | | | 3.30 | |
| | growth factor 18 | | | | | | | | |
| 1428942_PM_at | metallothionein 2 | Mt2 | 17750 | | 2.34 | | | 2.71 | |
| 1440882_PM_at | low density | Lrp8 | 16975 | | 2.13 | | | 2.19 | |
| | lipoprotein | | | | | | | | |
| | receptor-related | | | | | | | | |
| | protein 8, | | | | | | | | |
| | apolipoprotein e | | | | | | | | |
| | receptor | | | | | | | | |
| 1451751_PM_at | DNA-damage-inducible | Ddit4l | 73284 | | 2.11 | | | 1.85 | |
| | transcript 4-like | | | | | | | | |
| 1420696_PM_at | sema domain, | Sema3c | 20348 | | 1.62 | | | 1.66 | |
| | immunoglobulin | | | | | | | | |
| | domain (Ig), | | | | | | | | |
| | short basic domain, | | | | | | | | |
| | secreted, | | | | | | | | |
| | (semaphorin) 3C | | | | | | | | |
| 1460632_PM_at | retinal dehydrogenase | Rdh10 | 98711 | | 1.59 | | | 1.68 | |
| | 10 (all-trans) | | | | | | | | |
| 1454992_PM_at | solute carrier family 7 | Slc7a1 | 11987 | | 1.57 | | | 1.62 | |
| | (cationic amino | | | | | | | | |
| | acid transporter, | | | | | | | | |
| | y+ system), member 1 | | | | | | | | |
| 1436791_PM_at | wingless-related MMTV | Wnt5a | 22418 | | 1.57 | | | 1.69 | |
| | integration site 5A | | | | | | | | |
| 1456524_PM_at | neuregulin 1 | Nrg1 | 211323 | | 1.56 | | | 1.81 | |
| 1453474_PM_at | abhydrolase | Abhd15 | 67477 | | 1.56 | | | 1.64 | |
| | domain | | | | | | | | |
| | containing 15 | | | | | | | | |
| 1456888_PM_at | 6-phosphofructo- | Pfkfb4 | 270198 | | 1.55 | | | 1.69 | |
| | 2-kinase/fructose- | | | | | | | | |
| | 2,6-biphosphatase 4 | | | | | | | | |
| 1426887_PM_at | nudix (nucleoside | Nudt11 | 58242 | | 1.55 | | | 1.62 | |
| | diphosphate | | | | | | | | |
| | linked moiety | | | | | | | | |
| | X)-type motif 11 | | | | | | | | |
| 1434456_PM_at | RUN domain | Rundc3b | 242819 | | 1.55 | | | 1.54 | |
| | containing 3B | | | | | | | | |
| 1451041_PM_at | Rho-associated | Rock2 | 19878 | | 1.54 | | | 1.60 | |
| | coiled-coil containing | | | | | | | | |
| | protein kinase 2 | | | | | | | | |
| 1427320_PM_at | coatomer protein | Copg2as2 | 1E+08 | | 1.53 | | | 1.56 | |
| | complex, | | | | | | | | |
| | subunit gamma | | | | | | | | |
| | 2, antisense 2 | | | | | | | | |
| 1451828_PM_a_at | acyl-CoA | Acsl4 | 50790 | | 1.53 | | | 1.78 | |
| | synthetase long- | | | | | | | | |
| | chain family | | | | | | | | |
| | member 4 | | | | | | | | |
| 1436210_PM_at | glycerol kinase 5 | Gk5 | 235533 | | 1.51 | | | 1.55 | |
| | (putative) | | | | | | | | |
| 1426399_PM_at | von Willebrand | Vwa1 | 246228 | | −1.51 | | | −1.69 | |
| | factor A domain | | | | | | | | |
| | containing 1 | | | | | | | | |
| 1421609_PM_a_at | camello-like 3 | Cml3 | 93674 | | −1.56 | | | −1.64 | |
| 1425009_PM_at | t-complex- | Tcte2 | 21646 | | −1.59 | | | −1.61 | |
| | associated testis | | | | | | | | |
| | expressed 2 | | | | | | | | |
| 1428427_PM_at | F-box and | Fbxl2 | 72179 | | −1.59 | | | −1.68 | |
| | leucine-rich | | | | | | | | |
| | repeat protein 2 | | | | | | | | |
| 1451548_PM_at | uridine | Upp2 | 76654 | | −3.28 | | | −4.00 | |
| | phosphorylase 2 | | | | | | | | |
| 1418847_PM_at | arginase type II | Arg2 | 11847 | | | 2.39 | | | 2.70 |
| 1417828_PM_at | aquaporin 8 | Aqp8 | 11833 | | | 2.15 | | | 3.41 |
| 1424638_PM_at | cyclin-dependent | Cdkn1a | 12575 | | | 2.02 | | | 2.73 |
| | kinase inhibitor | | | | | | | | |
| | 1A (P21) | | | | | | | | |
| 1450562_PM_at | lymphocyte antigen 6 | Ly6f | 17071 | | | 1.91 | | | 1.93 |
| | complex, locus F | | | | | | | | |
| 1423954_PM_at | complement component 3 | C3 | 12266 | | | 1.90 | | | 2.34 |
| 1452934_PM_at | transmembrane | Tmc5 | 74424 | | | 1.88 | | | 1.91 |
| | channel-like | | | | | | | | |
| | gene family 5 | | | | | | | | |
| 1459661_PM_at | doublecortin domain | Dcdc2a | 195208 | | | 1.76 | | | 1.69 |
| | containing 2a | | | | | | | | |
| 1458504_PM_at | zinc finger CCCH type | Zc3h12d | 237256 | | | 1.69 | | | 1.58 |
| | containing 12D | | | | | | | | |
| 1428988_PM_at | ATP-binding | Abcc3 | 76408 | | | 1.67 | | | 1.64 |
| | cassette, sub- | | | | | | | | |
| | family C (CFTR/MRP), | | | | | | | | |
| | member 3 | | | | | | | | |
| 1444487_PM_at | lecithin-retinol | Lrat | 79235 | | | 1.67 | | | 1.72 |
| | acyltransferase | | | | | | | | |
| | (phosphatidyl- | | | | | | | | |
| | choline-retinol-O- | | | | | | | | |
| | acyltransferase) | | | | | | | | |
| 1418649_PM_at | EGL nine homolog 3 | Egln3 | 112407 | | | 1.63 | | | 1.71 |
| | (C. elegans) | | | | | | | | |
| 1426047_PM_a_at | protein tyrosine | Ptprr | 19279 | | | 1.61 | | | 1.69 |
| | phosphatase, | | | | | | | | |
| | receptor type, R | | | | | | | | |
| 1416101_PM_a_at | histone cluster 1, H1c | Hist1h1c | 50708 | | | 1.60 | | | 1.82 |
| 1440058_PM_at | solute carrier | Slc22a14 | 382113 | | | 1.59 | | | 1.54 |
| | family 22 | | | | | | | | |
| | (organic cation | | | | | | | | |
| | transporter), | | | | | | | | |
| | member 14 | | | | | | | | |
| 1449002_PM_at | pleckstrin | Phlda3 | 27280 | | | 1.57 | | | 1.81 |
| | homology-like | | | | | | | | |
| | domain, family | | | | | | | | |
| | A, member 3 | | | | | | | | |
| 1448330_PM_at | glutathione | Gstm1 | 14862 | | | 1.54 | | | 1.51 |
| | S-transferase, mu 1 | | | | | | | | |
| 1424835_PM_at | glutathione | Gstm4 | 14865 | | | 1.52 | | | 1.70 |
| | S-transferase, mu 4 | | | | | | | | |
| 1418215_PM_at | meprin 1 beta | Mep1b | 17288 | | | −1.50 | | | −1.55 |
| 1434050_PM_at | vacuolar protein | Vps8 | 209018 | | | −1.56 | | | −1.84 |
| | sorting 8 homolog | | | | | | | | |
| | (S. cerevisiae) | | | | | | | | |
| 1422606_PM_at | C1q and tumor | C1qtnf3 | 81799 | | | −1.67 | | | −1.57 |
| | necrosis factor | | | | | | | | |
| | related protein 3 | | | | | | | | |
| 1456722_PM_at | chordin-like 1 | Chrdl1 | 83453 | | | −1.71 | | | −1.55 |
| 1437870_PM_at | solute carrier | Slco4c1 | 227394 | | | −1.90 | | | −1.69 |
| | organic anion | | | | | | | | |
| | transporter | | | | | | | | |
| | family, member 4C1 | | | | | | | | |
| 1419754_PM_at | myosin VA | Myo5a | 17918 | | | −2.68 | | | −3.09 |
| 1436521_PM_at | solute carrier | Slc36a2 | 246049 | 4.63 | | | | | |
| | family 36 | | | | | | | | |
| | (proton/amino | | | | | | | | |
| | acid symporter), | | | | | | | | |
| | member 2 | | | | | | | | |
| 1422612_PM_at | hexokinase 2 | Hk2 | 15277 | 2.72 | | | | | |
| 1438664_PM_at | protein kinase, | Prkar2b | 19088 | 2.43 | | | | | |
| | cAMP dependent | | | | | | | | |
| | regulatory, type | | | | | | | | |
| | II beta | | | | | | | | |
| 1434893_PM_at | ATPase, Na+/K+ | Atp1a2 | 98660 | 2.25 | | | | | |
| | transporting, alpha 2 | | | | | | | | |
| | polypeptide | | | | | | | | |
| 1423405_PM_at | tissue inhibitor of | Timp4 | 110595 | 1.91 | | | | | |
| | metalloproteinase 4 | | | | | | | | |
| 1453596_PM_at | inhibitor of | Id2 | 15902 | 1.83 | | | | | |
| | DNA binding 2 | | | | | | | | |
| 1456041_PM_at | sorting nexin 16 | Snx16 | 74718 | 1.75 | | | | | |
| 1436366_PM_at | protein phosphatase 1, | Ppp1r15b | 108954 | 1.72 | | | | | |
| | regulatory (inhibitor) | | | | | | | | |
| | subunit 15b | | | | | | | | |
| 1448170_PM_at | seven in absentia 2 | Siah2 | 20439 | 1.71 | | | | | |
| 1441190_PM_at | actin related | Arpc5l | 74192 | 1.70 | | | | | |
| | protein 2/3 complex, | | | | | | | | |
| | subunit 5-like | | | | | | | | |
| 1455657_PM_at | SMG1 homolog, | Smg1 | 233789 | 1.69 | | | | | |
| | phosphatidylinositol | | | | | | | | |
| | 3-kinase-related kinase | | | | | | | | |
| | (C. elegans) | | | | | | | | |
| 1418203_PM_at | phorbol-12- | Pmaip1 | 58801 | 1.68 | | | | | |
| | myristate-13- | | | | | | | | |
| | acetate-induced | | | | | | | | |
| | protein 1 | | | | | | | | |
| 1433944_PM_at | HECT domain | Hectd2 | 226098 | 1.67 | | | | | |
| | containing 2 | | | | | | | | |
| 1423170_PM_at | TAF7 RNA | Taf7 | 24074 | 1.66 | | | | | |
| | polymerase II, | | | | | | | | |
| | TATA box | | | | | | | | |
| | binding protein | | | | | | | | |
| | (TBP)- | | | | | | | | |
| | associated factor | | | | | | | | |
| 1452394_PM_at | cysteinyl-tRNA | Cars | 27267 | 1.63 | | | | | |
| | synthetase | | | | | | | | |
| 1441546_PM_at | Membrane protein, | Mpp6 | 56524 | 1.62 | | | | | |
| | palmitoylated 6 | | | | | | | | |
| | (MAGUK p55 | | | | | | | | |
| | subfamily member 6) | | | | | | | | |
| 1441955_PM_s_at | polyadenylate | Paip1 | 218693 | 1.61 | | | | | |
| | binding protein- | | | | | | | | |
| | interacting protein 1 | | | | | | | | |
| 1438578_PM_a_at | BTB (POZ) domain | Btbd10 | 68815 | 1.60 | | | | | |
| | containing 10 | | | | | | | | |
| 1418025_PM_at | basic helix-loop- | Bhlhe40 | 20893 | 1.59 | | | | | |
| | helix family, | | | | | | | | |
| | member e40 | | | | | | | | |
| 1459585_PM_at | growth arrest | Gas6 | 14456 | 1.59 | | | | | |
| | specific 6 | | | | | | | | |
| 1417479_PM_at | protein phosphatase 2, | Ppp2r3c | 59032 | 1.59 | | | | | |
| | regulatory | | | | | | | | |
| | subunit B″, gamma | | | | | | | | |
| 1455185_PM_s_at | PHD finger protein 16 | Phf16 | 382207 | 1.58 | | | | | |
| 1436871_PM_at | serine/arginine- | Srsf7 | 225027 | 1.57 | | | | | |
| | rich splicing factor 7 | | | | | | | | |
| 1452094_PM_at | procollagen-proline, 2- | P4ha1 | 18451 | 1.56 | | | | | |
| | oxoglutarate 4- | | | | | | | | |
| | dioxygenase (proline 4- | | | | | | | | |
| | hydroxylase), alpha 1 | | | | | | | | |
| | polypeptide | | | | | | | | |
| 1443116_PM_at | proteasome (prosome, | Psme4 | 103554 | 1.56 | | | | | |
| | macropain) | | | | | | | | |
| | activator subunit 4 | | | | | | | | |
| 1424380_PM_at | vacuolar protein | Vps37b | 330192 | 1.56 | | | | | |
| | sorting 37B (yeast) | | | | | | | | |
| 1456810_PM_at | vacuolar protein | Vps54 | 245944 | 1.54 | | | | | |
| | sorting 54 (yeast) | | | | | | | | |
| 1452218_PM_at | coiled-coil domain | Ccdc117 | 104479 | 1.54 | | | | | |
| | containing 117 | | | | | | | | |
| 1435904_PM_at | eukaryotic translation | Eif2c3 | 214150 | 1.53 | | | | | |
| | initiation factor 2C, 3 | | | | | | | | |
| 1442071_PM_at | ATP-binding | Abce1 | 24015 | 1.53 | | | | | |
| | cassette, sub- | | | | | | | | |
| | family E (OABP), | | | | | | | | |
| | member 1 | | | | | | | | |
| 1440346_PM_at | KDM1 lysine | Kdm6b | 216850 | 1.52 | | | | | |
| | (K)-specific | | | | | | | | |
| | demethylase 6B | | | | | | | | |
| 1423549_PM_at | solute carrier family 1 | Slc1a4 | 55963 | 1.52 | | | | | |
| | (glutamate/neutral | | | | | | | | |
| | amino acid transporter), | | | | | | | | |
| | member 4 | | | | | | | | |
| 1435912_PM_at | UBX domain protein 7 | Ubxn7 | 224111 | 1.51 | | | | | |
| 1423605_PM_a_at | transformed | Mdm2 | 17246 | 1.51 | | | | | |
| | mouse 3T3 cell | | | | | | | | |
| | double minute 2 | | | | | | | | |
| 1425656_PM_a_at | brain-specific | Baiap2 | 108100 | 1.51 | | | | | |
| | angiogenesis inhibitor | | | | | | | | |
| | 1-associated protein 2 | | | | | | | | |
| 1425287_PM_at | zinc finger protein 189 | Zfp189 | 230162 | 1.51 | | | | | |
| 1455904_PM_at | growth arrest | Gas5 /// | 100217446 /// | 1.51 | | | | | |
| | specific 5 /// | Snord47 | 14455 | | | | | | |
| | small nucleolar | | | | | | | | |
| | RNA, C/D box 47 | | | | | | | | |
| 1438535_PM_at | pleckstrin homology | Phip | 83946 | 1.50 | | | | | |
| | domain interacting | | | | | | | | |
| | protein | | | | | | | | |
| 1419140_PM_at | activin receptor IIB | Acvr2b | 11481 | −1.51 | | | | | |
| 1428975_PM_at | sushi domain | Susd3 | 66329 | −1.51 | | | | | |
| | containing 3 | | | | | | | | |
| 1437231_PM_at | SLIT and NTRK-like | Slitrk6 | 239250 | −1.52 | | | | | |
| | family, member 6 | | | | | | | | |
| 1452962_PM_at | transmembrane | Tmem25 | 71687 | −1.52 | | | | | |
| | protein 25 | | | | | | | | |
| 1438784_PM_at | B-cell leukemia/lymphoma | Bcl11b | 58208 | −1.52 | | | | | |
| | 11B | | | | | | | | |
| 1427369_PM_at | NLR family, pyrin domain | Nlrp6 | 101613 | −1.52 | | | | | |
| | containing 6 | | | | | | | | |
| 1455087_PM_at | DNA segment, | D7Ertd715e | 52480 | −1.53 | | | | | |
| | Chr 7, ERATO | | | | | | | | |
| | Doi 715, expressed | | | | | | | | |
| 1449813_PM_at | zinc finger protein 30 | Zfp30 | 22693 | −1.55 | | | | | |
| 1451692_PM_at | transmembrane | Tmco6 | 71983 | −1.57 | | | | | |
| | and coiled-coil domains 6 | | | | | | | | |
| 1419051_PM_at | OVO homolog-like 1 | Ovol1 | 18426 | −1.60 | | | | | |
| | (Drosophila) | | | | | | | | |
| 1424213_PM_at | UbiA prenyltransferase | Ubiad1 | 71707 | −1.61 | | | | | |
| | domain containing 1 | | | | | | | | |
| 1441198_PM_at | zinc finger protein 39 | Zfp39 | 22698 | −1.64 | | | | | |
| 1446303_PM_at | insulin-like | Igf1r | 16001 | −1.66 | | | | | |
| | growth factor I | | | | | | | | |
| | receptor | | | | | | | | |
| 1429456_PM_a_at | polymerase | Polr3e | 26939 | −1.69 | | | | | |
| | (RNA) III (DNA | | | | | | | | |
| | directed) | | | | | | | | |
| | polypeptide E | | | | | | | | |
| 1457548_PM_at | A disintegrin-like and | Adamts6 | 108154 | −1.83 | | | | | |
| | metallopeptidase | | | | | | | | |
| | (reprolysin type) | | | | | | | | |
| | with thrombospondin | | | | | | | | |
| | type 1 motif, 6 | | | | | | | | |
| 1449981_PM_a_at | N-acetyltransferase | Nat2 | 17961 | −2.01 | | | | | |
| | 2 (arylamine | | | | | | | | |
| | N-acetyltransferase) | | | | | | | | |
| 1452975_PM_at | alanine-glyoxylate | Agxt2l1 | 71760 | −2.10 | | | | | |
| | aminotransferase | | | | | | | | |
| | 2-like 1 | | | | | | | | |
| 1427056_PM_at | a disintegrin-like | Adamts15 | 235130 | −2.26 | | | | | |
| | and metallopeptidase | | | | | | | | |
| | (reprolysin type) | | | | | | | | |
| | with thrombospondin | | | | | | | | |
| | type 1 motif, 15 | | | | | | | | |
| 1427372_PM_at | cytochrome | Cyp27b1 | 13115 | −3.22 | | | | | |
| | P450, family 27, | | | | | | | | |
| | subfamily b, | | | | | | | | |
| | polypeptide 1 | | | | | | | | |
| 1427094_PM_at | polymerase | Pole2 | 18974 | | 1.75 | | | | |
| | (DNA directed), | | | | | | | | |
| | epsilon 2 (p59 | | | | | | | | |
| | subunit) | | | | | | | | |
| 1440899_PM_at | flavin containing | Fmo5 | 14263 | | −1.51 | | | | |
| | monooxygenase 5 | | | | | | | | |
| 1460196_PM_at | carbonyl reductase 1 | Cbr1 | 12408 | | | 1.80 | | | |
| 1421108_PM_at | camello-like 2 | Cml2 | 93673 | | | 1.60 | | | |
| 1421603_PM_a_at | carcinoembryonic | Ceacam2 | 26367 | | | −1.68 | | | |
| | antigen-related | | | | | | | | |
| | cell adhesion | | | | | | | | |
| | molecule 2 | | | | | | | | |
| 1439348_PM_at | S100 calcium | S100a10 | 20194 | | | | 1.89 | | |
| | binding protein | | | | | | | | |
| | A10 (calpactin) | | | | | | | | |
| 1453775_PM_at | RPTOR | Rictor | 78757 | | | | 1.66 | | |
| | independent | | | | | | | | |
| | companion of | | | | | | | | |
| | MTOR, complex 2 | | | | | | | | |
| 1438073_PM_at | sprouty homolog | Spry3 | 236576 | | | | 1.61 | | |
| | 3 (Drosophila) | | | | | | | | |
| 1432108_PM_at | polycomb group | Pcgf6 | 71041 | | | | 1.56 | | |
| | ring finger 6 | | | | | | | | |
| 1443049_PM_at | Transmembrane | Tmem19 | 67226 | | | | 1.56 | | |
| | protein 19 | | | | | | | | |
| 1456225_PM_x_at | tribbles homolog | Trib3 | 228775 | | | | 1.56 | | |
| | 3 (Drosophila) | | | | | | | | |
| 1442959_PM_at | baculoviral IAP | Birc6 | 12211 | | | | 1.52 | | |
| | repeat-containing 6 | | | | | | | | |
| 1429204_PM_at | calcium/calmodulin- | Camk2n2 | 73047 | | | | 1.51 | | |
| | dependent | | | | | | | | |
| | protein kinase II | | | | | | | | |
| | inhibitor 2 | | | | | | | | |
| 1423672_PM_at | tetratricopeptide | Ttc30b | 72421 | | | | −1.51 | | |
| | repeat domain 30B | | | | | | | | |
| 1426029_PM_a_at | nuclear factor of | Nfat5 | 54446 | | | | −1.51 | | |
| | activated T-cells 5 | | | | | | | | |
| 1429943_PM_at | chitobiase, | Ctbs | 74245 | | | | −1.51 | | |
| | di-N-acetyl- | | | | | | | | |
| 1443054_PM_at | potassium inwardly- | Kcnj15 | 16516 | | | | −1.51 | | |
| | rectifying channel, | | | | | | | | |
| | subfamily J, member 15 | | | | | | | | |
| 1440779_PM_s_at | solute carrier family 5 | Slc5a9 | 230612 | | | | −1.52 | | |
| | (sodium/glucose | | | | | | | | |
| | cotransporter), | | | | | | | | |
| | member 9 | | | | | | | | |
| 1431726_PM_a_at | transmembrane | Tmem80 | 71448 | | | | −1.52 | | |
| | protein 80 | | | | | | | | |
| 1439300_PM_at | cysteine-rich | Chic1 | 12212 | | | | −1.53 | | |
| | hydrophobic domain 1 | | | | | | | | |
| 1418500_PM_at | nucleosome | Nap113 | 54561 | | | | −1.53 | | |
| | assembly protein | | | | | | | | |
| | 1-like 3 | | | | | | | | |
| 1424508_PM_at | tetratricopeptide | Ttc5 | 219022 | | | | −1.55 | | |
| | repeat domain 5 | | | | | | | | |
| 1458176_PM_at | Period homolog | Per3 | 18628 | | | | −1.57 | | |
| | 3 (Drosophila) | | | | | | | | |
| 1448494_PM_at | growth arrest | Gas1 | 14451 | | | | −1.57 | | |
| | specific 1 | | | | | | | | |
| 1422138_PM_at | plasminogen activator, | Plau | 18792 | | | | −1.58 | | |
| | urokinase | | | | | | | | |
| 1436870_PM_s_at | actin filament | Afap1l2 | 226250 | | | | −1.62 | | |
| | associated | | | | | | | | |
| | protein 1-like 2 | | | | | | | | |
| 1430834_PM_at | GPRIN family | Gprin3 | 243385 | | | | −1.62 | | |
| | member 3 | | | | | | | | |
| 1416497_PM_at | protein disulfide | Pdia4 | 12304 | | | | −1.66 | | |
| | isomerase associated 4 | | | | | | | | |
| 1456070_PM_at | protein tyrosine | Ptprg | 19270 | | | | −2.31 | | |
| | phosphatase, | | | | | | | | |
| | receptor type, G | | | | | | | | |
| 1421816_PM_at | glutathione reductase | Gsr | 14782 | | | | | 1.83 | |
| 1455902_PM_x_at | Ras homolog gene family, | Rhof | 23912 | | | | | 1.66 | |
| | member f | | | | | | | | |
| 1429262_PM_at | Ras association | Rassf6 | 73246 | | | | | 1.65 | |
| | (RalGDS/AF-6) | | | | | | | | |
| | domain family member 6 | | | | | | | | |
| 1442051_PM_at | histone cluster 2, H3c1 | Hist2h3c1 | 15077 | | | | | 1.65 | |
| 1423723_PM_s_at | TAR DNA | Tardbp | 230908 | | | | | 1.62 | |
| | binding protein | | | | | | | | |
| 1442018_PM_at | B-cell translocation | Btg1 | 12226 | | | | | 1.60 | |
| | gene 1, anti- | | | | | | | | |
| | proliferative | | | | | | | | |
| 1416563_PM_at | cytidine 5′- | Ctps | 51797 | | | | | 1.57 | |
| | triphosphate | | | | | | | | |
| | synthase | | | | | | | | |
| 1434976_PM_x_at | eukaryotic translation | Eif4ebp1 | 13685 | | | | | 1.56 | |
| | initiation factor | | | | | | | | |
| | 4E binding protein 1 | | | | | | | | |
| 1417884_PM_at | solute carrier | Slc16a6 | 104681 | | | | | 1.55 | |
| | family 16 | | | | | | | | |
| | (monocarboxylic | | | | | | | | |
| | acid transporters), | | | | | | | | |
| | member 6 | | | | | | | | |
| 1457707_PM_at | multiple C2 domains, | Mctp2 | 244049 | | | | | 1.54 | |
| | transmembrane 2 | | | | | | | | |
| 1442116_PM_at | G protein- | Gpr176 | 381413 | | | | | 1.53 | |
| | coupled receptor 176 | | | | | | | | |
| 1434775_PM_at | par-3 (partitioning | Pard3 | 93742 | | | | | 1.53 | |
| | defective 3) | | | | | | | | |
| | homolog | | | | | | | | |
| | (C. elegans) | | | | | | | | |
| 1427347_PM_s_at | tubulin, beta 2A | Tubb2a | 22151 | | | | | 1.52 | |
| 1423413_PM_at | N-myc downstream | Ndrg1 | 17988 | | | | | 1.52 | |
| | regulated gene 1 | | | | | | | | |
| 1426275_PM_a_at | UDP-glucuronate | Uxs1 | 67883 | | | | | 1.50 | |
| | decarboxylase 1 | | | | | | | | |
| 1448700_PM_at | G0/G1 switch gene 2 | G0s2 | 14373 | | | | | 1.50 | |
| 1449220_PM_at | GTPase, IMAP | Gimap3 | 83408 | | | | | −1.51 | |
| | family member 3 | | | | | | | | |
| 1425778_PM_at | indoleamine | Ido2 | 209176 | | | | | −1.52 | |
| | 2,3-dioxygenase 2 | | | | | | | | |
| 1417932_PM_at | interleukin 18 | Il18 | 16173 | | | | | −1.52 | |
| 1439866_PM_at | cullin 9 | Cul9 | 78309 | | | | | −1.52 | |
| 1430530_PM_s_at | NmrA-like | Nmral1 | 67824 | | | | | −1.52 | |
| | family domain | | | | | | | | |
| | containing 1 | | | | | | | | |
| 1434990_PM_at | protein phosphatase 1E | Ppm1e | 320472 | | | | | −1.53 | |
| | (PP2C domain | | | | | | | | |
| | containing) | | | | | | | | |
| 1426230_PM_at | sphingosine | Sphk2 | 56632 | | | | | −1.54 | |
| | kinase 2 | | | | | | | | |
| 1428758_PM_at | transmembrane | Tmem86a | 67893 | | | | | −1.56 | |
| | protein 86A | | | | | | | | |
| 1417700_PM_at | RAB38, member | Rab38 | 72433 | | | | | −1.57 | |
| | of RAS | | | | | | | | |
| | oncogene family | | | | | | | | |
| 1426767_PM_at | WD repeat domain 90 | Wdr90 | 106618 | | | | | −1.58 | |
| 1435994_PM_at | potassium | Kcnh1 | 16510 | | | | | −1.61 | |
| | voltage-gated channel, | | | | | | | | |
| | subfamily H | | | | | | | | |
| | (eag-related), | | | | | | | | |
| | member 1 | | | | | | | | |
| 1457038_PM_at | Fras1 related | Frem2 | 242022 | | | | | −1.66 | |
| | extracellular | | | | | | | | |
| | matrix protein 2 | | | | | | | | |
| 1418174_PM_at | D site albumin promoter | Dbp | 13170 | | | | | −1.73 | |
| | binding protein | | | | | | | | |
| 1435918_PM_at | family with sequence | Fam107a | 268709 | | | | | −1.77 | |
| | similarity 107, | | | | | | | | |
| | member A | | | | | | | | |
| 1416468_PM_at | aldehyde dehydrogenase | Aldh1a1 | 11668 | | | | | | 2.21 |
| | family 1, subfamily A1 | | | | | | | | |
| 1418706_PM_at | solute carrier family 38, | Slc38a3 | 76257 | | | | | | 1.81 |
| | member 3 | | | | | | | | |
| 1424279_PM_at | fibrinogen alpha chain | Fga | 14161 | | | | | | 1.79 |
| 1423596_PM_at | NIMA (never in | Nek6 | 59126 | | | | | | 1.69 |
| | mitosis gene a)-related | | | | | | | | |
| | expressed kinase 6 | | | | | | | | |
| 1443964_PM_at | transmembrane inner ear | Tmie | 20776 | | | | | | 1.65 |
| 1444242_PM_at | Solute carrier | Slco2a1 | 24059 | | | | | | 1.63 |
| | organic anion | | | | | | | | |
| | transporter | | | | | | | | |
| | family, member 2a1 | | | | | | | | |
| 1421040_PM_a_at | glutathione | Gsta2 | 14858 | | | | | | 1.61 |
| | S-transferase, | | | | | | | | |
| | alpha 2 (Yc2) | | | | | | | | |
| 1435121_PM_at | deiodinase, | Dio3os | 353504 | | | | | | 1.60 |
| | iodothyronine | | | | | | | | |
| | type III, | | | | | | | | |
| | opposite strand | | | | | | | | |
| 1449065_PM_at | acyl-CoA | Acot1 | 26897 | | | | | | 1.59 |
| | thioesterase 1 | | | | | | | | |
| 1417150_PM_at | solute carrier family 6 | Slc6a4 | 15567 | | | | | | 1.58 |
| | (neurotransmitter | | | | | | | | |
| | transporter, | | | | | | | | |
| | serotonin), member 4 | | | | | | | | |
| 1451607_PM_at | kallikrein | Klk1b21 | 16616 | | | | | | 1.57 |
| | 1-related peptidase b21 | | | | | | | | |
| 1437932_PM_a_at | claudin 1 | Cldn1 | 12737 | | | | | | 1.57 |
| 1449575_PM_a_at | glutathione | Gstp1 | 14870 | | | | | | 1.54 |
| | S-transferase, pi 1 | | | | | | | | |
| 1440965_PM_at | phosphatidylinositol | Pigl | 327942 | | | | | | 1.52 |
| | glycan anchor | | | | | | | | |
| | biosynthesis, class L | | | | | | | | |
| 1430128_PM_a_at | receptor accessory | Reep6 | 70335 | | | | | | 1.51 |
| | protein 6 | | | | | | | | |
| 1422997_PM_s_at | acyl-CoA | Acot1 /// | 171210 /// | | | | | | 1.51 |
| | thioesterase 1 /// | Acot2 | 26897 | | | | | | |
| | acyl-CoA | | | | | | | | |
| | thioesterase 2 | | | | | | | | |
| 1418746_PM_at | paroxysmal | Pnkd | 56695 | | | | | | 1.51 |
| | nonkinesiogenic | | | | | | | | |
| | dyskinesia | | | | | | | | |
| 1420654_PM_a_at | glucan (1,4-alpha-), | Gbe1 | 74185 | | | | | | 1.51 |
| | branching | | | | | | | | |
| | enzyme 1 | | | | | | | | |
| 1422966_PM_a_at | transferring receptor | Tfrc | 22042 | | | | | | 1.51 |
| 1426502_PM_s_at | glutamic pyruvic | Gpt | 76282 | | | | | | 1.51 |
| | transaminase, soluble | | | | | | | | |
| 1436291_PM_a_at | dihydropyrimidinase | Dpys | 64705 | | | | | | 1.51 |
| 1460629_PM_at | tripartite motif- | Trim16 | 94092 | | | | | | 1.51 |
| | containing 16 | | | | | | | | |
| 1421756_PM_a_at | G protein- | Gpr19 | 14760 | | | | | | 1.50 |
| | coupled receptor 19 | | | | | | | | |
| 1431172_PM_at | origin recognition | Orc4 | 26428 | | | | | | 1.50 |
| | complex, subunit 4 | | | | | | | | |
| 1440227_PM_at | solute carrier | Slc5a3 | 53881 | | | | | | −1.50 |
| | family 5 (inositol | | | | | | | | |
| | transporters), | | | | | | | | |
| | member 3 | | | | | | | | |
| 1433617_PM_s_at | UDP-Gal:betaGlcNAc | B4galt5 | 56336 | | | | | | −1.51 |
| | beta 1,4- | | | | | | | | |
| | galactosyltransferase, | | | | | | | | |
| | polypeptide 5 | | | | | | | | |
| 1428012_PM_at | complement component 8, | C8a | 230558 | | | | | | −1.51 |
| | alpha polypeptide | | | | | | | | |
| 1428343_PM_at | REST corepressor 3 | Rcor3 | 214742 | | | | | | −1.51 |
| 1427224_PM_at | acyl-CoA synthetase | Acsm2 | 233799 | | | | | | −1.52 |
| | medium-chain | | | | | | | | |
| | family member 2 | | | | | | | | |
| 1448482_PM_at | solute carrier | Slc39a8 | 67547 | | | | | | −1.55 |
| | family 39 (metal | | | | | | | | |
| | ion transporter), | | | | | | | | |
| | member 8 | | | | | | | | |
| 1440688_PM_at | Rho GTPase activating | Arhgap26 | 71302 | | | | | | −1.57 |
| | protein 26 | | | | | | | | |
| 1452333_PM_at | SWI/SNF related, matrix | Smarca2 | 67155 | | | | | | −1.60 |
| | associated, actin | | | | | | | | |
| | dependent regulator of | | | | | | | | |
| | chromatin, subfamily a, | | | | | | | | |
| | member 2 | | | | | | | | |
| 1421167_PM_at | ATPase, class | Atp11a | 50770 | | | | | | −1.62 |
| | VI, type 11A | | | | | | | | |
| 1449610_PM_at | E1A binding | Ep400 | 75560 | | | | | | −1.67 |
| | protein p400 | | | | | | | | |
| 1446742_PM_at | Nuclear factor I/A | Nfia | 18027 | | | | | | −1.74 |
| 1437675_PM_at | solute carrier family 8 | Slc8a1 | 20541 | | | | | | −1.86 |
| | (sodium/calcium | | | | | | | | |
| | exchanger), member 1 | | | | | | | | |
| 1437473_PM_at | avian musculoaponeurotic | Maf | 17132 | | | | −1.56 | | 1.53 |
| | fibrosarcoma | | | | | | | | |
| | (v-maf) AS42 | | | | | | | | |
| | oncogene homolog | | | | | | | | |
| 1441944_PM_s_at | G protein- | Gpr135 | 238252 | | | | | 1.66 | 1.59 |
| | coupled receptor 135 | | | | | | | | |
| 1460196_PM_at | carbonyl reductase 1 | Cbr1 | 12408 | | | | | 1.58 | 2.10 |
| 1450410_PM_a_at | solute carrier | Slc48a1 | 67739 | | | | | 1.58 | 1.53 |
| | family 48 (heme | | | | | | | | |
| | transporter), | | | | | | | | |
| | member 1 | | | | | | | | |
| 1439695_PM_a_at | kinesin family | Kif20b | 240641 | | | | | −2.61 | −2.85 |
| | member 20B | | | | | | | | |

Liver:

| | | | | DMF | DMF | DMF | MMF | MMF | MMF |
|---|---|---|---|---|---|---|---|---|---|
| Probe Set | Gene Title | Gene | | 2 hr | 7 hr | 12 hr | 2 hr | 7 hr | 12 hr |
| 1448894_PM_at | aldo-keto reductase | Akr1b8 | 14187 | 2.18 | 1.58 | | 2.27 | 1.75 | |
| | family 1, member B8 | | | | | | | | |
| 1419627_PM_s_at | C-type lectin domain | Clec4n | 56620 | 2.34 | 2.01 | | | 2.40 | |
| | family 4, member n | | | | | | | | |
| 1419942_PM_at | Sulfiredoxin 1 homolog | Srxn1 | 76650 | 1.69 | | | 1.68 | 1.52 | |
| | (S. cerevisiae) | | | | | | | | |
| 1417801_PM_a_at | PTPRF interacting | Ppfibp2 | 19024 | 2.38 | | | 2.55 | | |
| | protein, binding | | | | | | | | |
| | protein 2 | | | | | | | | |
| | (liprin beta 2) | | | | | | | | |
| 1448239_PM_at | heme oxygenase | Hmox1 | 15368 | 2.37 | | | 2.41 | | |
| | (decycling) 1 | | | | | | | | |
| 1420804_PM_s_at | C-type lectin domain | Clec4d | 17474 | 2.30 | | | 2.05 | | |
| | family 4, member d | | | | | | | | |
| 1438953_PM_at | c-fos induced | Figf | 14205 | 2.23 | | | 2.25 | | |
| | growth factor | | | | | | | | |
| 1452233_PM_at | ATP-binding | Abcc1 | 17250 | 1.75 | | | 1.86 | | |
| | cassette, sub- | | | | | | | | |
| | family C | | | | | | | | |
| | (CFTR/MRP), | | | | | | | | |
| | member 1 | | | | | | | | |
| 1436771_PM_x_at | phosphogluconate | Pgd | 110208 | 1.59 | | | 1.61 | | |
| | dehydrogenase | | | | | | | | |
| 1423627_PM_at | NAD(P)H dehydrogenase, | Nqo1 | 18104 | | 1.81 | | | 2.20 | |
| | quinone 1 | | | | | | | | |
| 1450759_PM_at | bone morphogenetic | Bmp6 | 12161 | | 1.57 | | | 1.65 | |
| | protein 6 | | | | | | | | |
| 1435495_PM_at | adenosine A1 | Adora1 | 11539 | | 1.50 | | | | |
| | receptor | | | | | | | | |
| 1424835_PM_at | glutathione | Gstm4 | 14865 | | | 1.12 | | | |
| | S-transferase, mu 4 | | | | | | | | |
| 1424296_PM_at | glutamate-cysteine | Gclc | 14629 | | | | 1.72 | | |
| | ligase, catalytic | | | | | | | | |
| | subunit | | | | | | | | |
| 1422573_PM_at | adenosine monophosphate | Ampd3 | 11717 | | | | 1.70 | | |
| | deaminase 3 | | | | | | | | |
| 1455016_PM_at | PRP38 pre-mRNA | Prpf38b | 66921 | | | | −1.53 | | |
| | processing factor 38 | | | | | | | | |
| | (yeast) domain | | | | | | | | |
| | containing B | | | | | | | | |
| 1452247_PM_at | fragile X mental | Fxr1 | 14359 | | | | −1.53 | | |
| | retardation gene 1, | | | | | | | | |
| | autosomal homolog | | | | | | | | |
| 1447019_PM_at | cytidine | Cmah | 12763 | | | | −1.55 | | |
| | monophospho-N- | | | | | | | | |
| | acetylneuraminic acid | | | | | | | | |
| | hydroxylase | | | | | | | | |
| 1448348_PM_at | cell cycle associated | Caprin1 | 53872 | | | | −1.56 | | |
| | protein 1 | | | | | | | | |
| 1419038_PM_a_at | casein kinase 2, alpha 1 | Csnk2a1 | 12995 | | | | −1.62 | | |
| | polypeptide | | | | | | | | |
| 1440091_PM_at | Meis homeobox 2 | Meis2 | 17536 | | | | −1.67 | | |
| 1416734_PM_at | muskelin 1, | Mkln1 | 27418 | | | | −1.69 | | |
| | intracellular mediator | | | | | | | | |
| | containing kelch motifs | | | | | | | | |
| 1453908_PM_at | protein tyrosine | Ptprb | 19263 | | | | −1.76 | | |
| | phosphatase, | | | | | | | | |
| | receptor type, B | | | | | | | | |
| 1456070_PM_at | protein tyrosine | Ptprg | 19270 | | | | −1.85 | | |
| | phosphatase, | | | | | | | | |
| | receptor type, G | | | | | | | | |
| 1449498_PM_at | macrophage | Marco | 17167 | | | | | 2.44 | |
| | receptor with | | | | | | | | |
| | collagenous structure | | | | | | | | |
| 1427357_PM_at | cytidine deaminase | Cda | 72269 | | | | | 1.92 | |
| 1448354_PM_at | glucose-6-phosphate | G6pdx | 14381 | | | | | 1.61 | |
| | dehydrogenase | | | | | | | | |
| | X-linked | | | | | | | | |
| 1435040_PM_at | interleukin-1 | Irak3 | 73914 | | | | | 1.53 | |
| | receptor-associated | | | | | | | | |
| | kinase 3 | | | | | | | | |
| 1429001_PM_at | pirin | Pir | 69656 | | | | | 1.50 | |
| 1445815_PM_at | frizzled homolog 8 | Fzd8 | 14370 | | | | | −1.81 | |
| | (Drosophila) | | | | | | | | |
| 1434582_PM_at | ELKS/RAB6-interacting/ | Erc2 | 238988 | | | | | −1.95 | |
| | CAST family member 2 | | | | | | | | |
| 1419669_PM_at | proteinase 3 | Prtn3 | 19152 | | | | | | 4.46 |
| 1431214_PM_at | predicted gene 3579 | Gm3579 | 1E+08 | | | | | | 2.74 |
| 1417441_PM_at | DnaJ (Hsp40) homolog, | Dnajc12 | 30045 | | | | | | 2.53 |
| | subfamily C, member 12 | | | | | | | | |
| 1428154_PM_s_at | phosphatidic acid | Ppapdc1b | 71910 | | | | | | 1.87 |
| | phosphatase type 2 | | | | | | | | |
| | domain containing 1B | | | | | | | | |
| 1444176_PM_at | ATPase, H+ transporting, | Atp6v0d2 | 242341 | | | | | | 1.84 |
| | lysosomal V0 subunit D2 | | | | | | | | |
| 1423290_PM_at | hypoxia up-regulated 1 | Hyou1 | 12282 | | | | | | 1.82 |
| 1433833_PM_at | fibronectin type III | Fndc3b | 72007 | | | | | | 1.66 |
| | domain containing 3B | | | | | | | | |
| 1416235_PM_at | leucine rich repeat | Lrrc59 | 98238 | | | | | | 1.65 |
| | containing 59 | | | | | | | | |
| 1448471_PM_a_at | cytotoxic T lymphocyte- | Ctla2a | 13024 | | | | | | 1.63 |
| | associated protein 2 | | | | | | | | |
| | alpha | | | | | | | | |
| 1448574_PM_at | non-metastatic cells 6, | Nme6 | 54369 | | | | | | 1.57 |
| | protein expressed in | | | | | | | | |
| | (nucleoside-diphosphate | | | | | | | | |
| | kinase) | | | | | | | | |
| 1443807_PM_x_at | cyclin F | Ccnf | 12449 | | | | | | 1.57 |
| 1450971_PM_at | growth arrest and DNA- | Gadd45b | 17873 | | | | | | 1.50 |
| | damage-inducible 45 beta | | | | | | | | |
| 1453103_PM_at | actin-binding | Ablim1 | 226251 | | | | | | −1.51 |
| | LIM protein 1 | | | | | | | | |
| 1439117_PM_at | calmin | Clmn | 94040 | | | | | | −1.54 |
| 1441988_PM_at | protein phosphatase | Ppm1k | 243382 | | | | | | −1.56 |
| | 1K (PP2C domain | | | | | | | | |
| | containing) | | | | | | | | |
| 1460256_PM_at | carbonic anhydrase 3 | Car3 | 12350 | | | | | | −1.57 |

Spleen:

| | | | | DMF | DMF | DMF | MMF | MMF | MMF |
|---|---|---|---|---|---|---|---|---|---|
| Probe Set | Gene Title | Gene | Entrez | 2 hr | 7 hr | 12 hr | 2 hr | 7 hr | 12 hr |
| 1419942_PM_at | Sulfiredoxin 1 homolog | Srxn1 | 76650 | 2.16 | 1.67 | | 2.02 | 1.87 | |
| | (S. cerevisiae) | | | | | | | | |
| 1419435_PM_at | aldehyde oxidase 1 | Aox1 | 11761 | 2.61 | | | 2.35 | | |
| 1434797_PM_at | kin of IRRE like | Kirrel | 170643 | −1.54 | | | −1.65 | | |
| | (Drosophila) | | | | | | | | |
| 1424022_PM_at | oxidative stress | Osgin1 | 71839 | 1.78 | | | 1.83 | | |
| | induced growth | | | | | | | | |
| | inhibitor 1 | | | | | | | | |
| 1416497_PM_at | protein disulfide | Pdia4 | 12304 | −1.60 | | | −1.59 | | |
| | isomerase associated 4 | | | | | | | | |
| 1424486_PM_a_at | thioredoxin reductase 1 | Txnrd1 | 50493 | 2.41 | | | 2.14 | | |
| 1424296_PM_at | glutamate- | Gclc | 14629 | 1.76 | | | | | |
| | cysteine ligase, | | | | | | | | |
| | catalytic subunit | | | | | | | | |
| 1448916_PM_at | v-maf musculoaponeurotic | Mafg | 17134 | 1.58 | | | | | |
| | fibrosarcoma oncogene | | | | | | | | |
| | family, protein | | | | | | | | |
| | G (avian) | | | | | | | | |
| 1434951_PM_at | armadillo repeat | Armc8 | 74125 | | | | −1.54 | | |
| | containing 8 | | | | | | | | |
| 1425521_PM_at | polyadenylate binding | Paip1 | 218693 | | | | −1.56 | | |
| | protein-interacting | | | | | | | | |
| | protein 1 | | | | | | | | |
| 1424084_PM_at | ROD1 regulator of | Rod1 | 230257 | | | | −1.54 | | |
| | differentiation | | | | | | | | |
| | 1 (S. pombe) | | | | | | | | |

APPENDIX B
(NAÏVE SINGLE DOSE GENE LISTS)

| | | | | | |
|---|---|---|---|---|---|
| | Gene | Entrez | | | |
| Gene Title | Symbol | Gene | FC | p.value | lods |

WHOLE BLOOD
100DMF-vs-Veh, 2 h

| | | | | | | |
|---|---|---|---|---|---|---|
| 1420937_PM_at | cleavage and polyadenylation specific | Cpsf2 | 51786 | −1.65 | 0.00 | 0.39 |
| | factor 2 | | | | | |
| 1441373_PM_at | — | — | — | −1.70 | 0.00 | 1.48 |

100DMF-vs-Veh, 7 h
None
100DMF-vs-Veh, 12 h

| | | | | | | |
|---|---|---|---|---|---|---|
| 1417898_PM_a_at | granzyme A | Gzma | 14938 | 1.92 | 0.00 | 1.91 |
| 1422089_PM_at | natural cytotoxicity triggering | Ncr1 | 17086 | 1.86 | 0.00 | 1.49 |
| | receptor 1 | | | | | |
| 1425005_PM_at | killer cell lectin-like receptor | Klrc1 | 16641 | 1.85 | 0.00 | 2.42 |
| | subfamily C, member 1 | | | | | |
| 1445399_PM_at | killer cell lectin-like receptor | Klrb1b | 80782 | 1.65 | 0.00 | 0.23 |
| | subfamily B member 1B | | | | | |
| 1458642_PM_at | killer cell lectin-like receptor family | Klre1 | 243655 | 1.85 | 0.00 | 0.87 |
| | E member 1 | | | | | |

100MMF-vs-Veh, 2 h

| | | | | | | |
|---|---|---|---|---|---|---|
| 1418133_PM_at | B-cell leukemia/lymphoma 3 | Bcl3 | 12051 | −1.67 | 0.00 | 1.65 |

100MMF-vs-Veh, 7 h
None
100MMF-vs-Veh, 12 h

| | | | | | | |
|---|---|---|---|---|---|---|
| 1415943_PM_at | syndecan 1 | Sdc1 | 20969 | −1.96 | 0.00 | 0.11 |
| 1435109_PM_at | RIKEN cDNA 0710007G10 | 0710007G10Rik | 68409 /// | −1.52 | 0.00 | 0.66 |
| | gene /// transmembrane protein | /// | 72392 | | | |
| | 175 | Tmem175 | | | | |

100DMF-vs-100MMF, 2 h
None
100DMF-vs-100MMF, 7 h

| | | | | | | |
|---|---|---|---|---|---|---|
| 1444037_PM_at | lectin, mannose- | Lman1 | 70361 | 1.63 | 0.00 | 0.41 |
| | binding, 1 | | | | | |

100DMF-vs-100MMF, 12 h
None
CEREBELLUM
100DMF-vs-Veh, 2 h
None
100DMF-vs-Veh, 7 h
None
100DMF-vs-Veh, 12 h
None
100MMF-vs-Veh, 2 h

| | | | | | | |
|---|---|---|---|---|---|---|
| 1419034_PM_at | casein kinase 2, alpha 1 | Csnk2a1 | 12995 | −1.65 | 0.00 | 1.07 |
| | polypeptide | | | | | |
| 1429607_PM_at | trafficking protein, kinesin | Trak2 | 70827 | −1.55 | 0.00 | 2.91 |
| | binding 2 | | | | | |

100MMF-vs-Veh, 7 h
None
100MMF-vs-Veh, 12 h

| | | | | | | |
|---|---|---|---|---|---|---|
| 1425428_PM_at | hypoxia inducible factor 3, | Hif3a | 53417 | 1.58 | 0.00 | 1.07 |
| | alpha subunit | | | | | |
| 1447845_PM_s_at | vanin 1 | Vnn1 | 22361 | 1.83 | 0.00 | 0.77 |
| 1451361_PM_a_at | patatin-like phospholipase | Pnpla7 | 241274 | 1.52 | 0.00 | 0.30 |
| | domain containing 7 | | | | | |

100DMF-vs-100MMF, 2 h
None
100DMF-vs-100MMF, 7 h
None
100DMF-vs-100MMF, 12 h
None
CORTEX
100DMF-vs-Veh, 2 h

| | | | | | | |
|---|---|---|---|---|---|---|
| 1442075_PM_at | expressed sequence | AI314604 | 102027 | 1.57 | 0.00 | 3.18 |
| | AI314604 | | | | | |
| 1448140_PM_at | cytokine induced apoptosis | Ciapin1 | 109006 | 1.55 | 0.00 | 0.90 |
| | inhibitor 1 | | | | | |

100DMF-vs-Veh, 7 h

| | | | | | | |
|---|---|---|---|---|---|---|
| 1419086_PM_at | fibroblast growth factor | Fgfbp1 | 14181 | 1.54 | 0.00 | 3.04 |
| | binding protein 1 | | | | | |
| 1423627_PM_at | NAD(P)H dehydrogenase, | Nqo1 | 18104 | 1.50 | 0.00 | 8.91 |
| | quinone 1 | | | | | |

100DMF-vs-Veh, 12 h
None
100MMF-vs-Veh, 2 h

| | | | | | | |
|---|---|---|---|---|---|---|
| 1416734_PM_at | muskelin 1, intracellular mediator | Mkln1 | 27418 | −1.85 | 0.00 | 0.10 |
| | containing kelch motifs | | | | | |
| 1420670_PM_at | aryl hydrocarbon receptor nuclear | Arnt2 | 11864 | −1.66 | 0.00 | 2.56 |
| | translocator 2 | | | | | |
| 1421166_PM_at | attractin | Atrn | 11990 | −1.62 | 0.00 | 1.39 |
| 1423594_PM_a_at | endothelin receptor type B | Ednrb | 13618 | −1.53 | 0.00 | 1.57 |
| 1429607_PM_at | trafficking protein, kinesin binding 2 | Trak2 | 70827 | −1.57 | 0.00 | 6.27 |
| 1430827_PM_a_at | PTK2 protein tyrosine kinase 2 | Ptk2 | 14083 | −1.53 | 0.00 | 0.41 |
| 1438108_PM_at | pleckstrin homology domain containing, | Plekhm3 | 241075 | −1.50 | 0.00 | 2.68 |
| | family M, member 3 | | | | | |
| 1443123_PM_at | tetratricopeptide repeat, ankyrin repeat | Tanc2 | 77097 | −1.72 | 0.00 | 0.53 |
| | and coiled-coil containing 2 | | | | | |
| 1448458_PM_at | topoisomerase (DNA) II beta | Top2b | 21974 | −1.51 | 0.00 | 1.27 |

100MMF-vs-Veh, 7 h

| | | | | | | |
|---|---|---|---|---|---|---|
| 1419086_PM_at | fibroblast growth factor | Fgfbp1 | 14181 | 1.62 | 0.00 | 5.18 |
| | binding protein 1 | | | | | |
| 1419435_PM_at | aldehyde oxidase 1 | Aox1 | 11761 | 1.65 | 0.00 | 8.09 |

100MMF-vs-Veh, 12 h

| | | | | | | |
|---|---|---|---|---|---|---|
| 1419435_PM_at | aldehyde oxidase 1 | Aox1 | 11761 | 1.54 | 0.00 | 4.12 |

100DMF-vs-100MMF, 2 h
None
100DMF-vs-100MMF, 7 h
None
100DMF-vs-100MMF, 12 h
None
HIPPOCAMPUS
100DMF-vs-Veh, 2 h
None
100DMF-vs-Veh, 7 h

| | | | | | | |
|---|---|---|---|---|---|---|
| 1443353_PM_at | — | — | — | −1.84 | 0.00 | 1.94 |
| 1458305_PM_at | transmembrane and | Tmtc3 | 237500 | −2.38 | 0.00 | 12.74 |
| | tetratricopeptide repeat containing 3 | | | | | |

100DMF-vs-Veh, 12 h
None
100MMF-vs-Veh, 2 h

| | | | | | | |
|---|---|---|---|---|---|---|
| 1417975_PM_at | karyopherin (importin) alpha 4 | Kpna4 | 16649 | −1.51 | 0.00 | 3.16 |
| 1434407_PM_at | SLIT-ROBO Rho GTPase | Srgap2 | 14270 | −1.50 | 0.00 | 0.09 |
| | activating protein 2 | | | | | |
| 1438085_PM_at | HEAT repeat containing 5B | Heatr5b | 320473 | −1.56 | 0.00 | 3.25 |

100MMF-vs-Veh, 7 h

| | | | | | | |
|---|---|---|---|---|---|---|
| 1443353_PM_at | — | — | — | −1.74 | 0.00 | 0.37 |
| 1458305_PM_at | transmembrane and | Tmtc3 | 237500 | −2.40 | 0.00 | 13.00 |
| | tetratricopeptide repeat | | | | | |
| | containing 3 | | | | | |

100MMF-vs-Veh, 12 h

| | | | | | | |
|---|---|---|---|---|---|---|
| 1435119_PM_at | hypothetical | LOC100502895 | 100502895 | 1.66 | 0.00 | 0.11 |
| | LOC100502895 | | | | | |

100DMF-vs-100MMF, 2 h

| | | | | | | |
|---|---|---|---|---|---|---|
| 1418938_PM_at | deiodinase, iodothyronine, type II | Dio2 | 13371 | 1.58 | 0.00 | 1.66 |
| 1458305_PM_at | transmembrane and tetratricopeptide | Tmtc3 | 237500 | −1.88 | 0.00 | 4.55 |
| | repeat containing 3 | | | | | |

100DMF-vs-100MMF, 7 h
None
100DMF-vs-100MMF, 12 h
None
STRIATUM
100DMF-vs-Veh, 2 h

| | | | | | | |
|---|---|---|---|---|---|---|
| 1416734_PM_at | muskelin 1, intracellular mediator | Mkln1 | 27418 | −1.97 | 0.00 | 0.74 |
| | containing kelch motifs | | | | | |
| 1428320_PM_at | KDM3B lysine (K)-specific | Kdm3b | 277250 | −1.54 | 0.00 | 1.72 |
| | demethylase 3B | | | | | |
| 1434030_PM_at | GTP-binding protein 10 (putative) | Gtpbp10 | 207704 | −1.52 | 0.00 | 1.47 |
| 1444791_PM_at | — | — | — | −1.60 | 0.00 | 0.93 |

100DMF-vs-Veh, 7 h

| | | | | | | |
|---|---|---|---|---|---|---|
| 1419435_PM_at | aldehyde oxidase 1 | Aox1 | 11761 | 1.64 | 0.00 | 1.07 |
| 1443353_PM_at | — | — | — | −1.83 | 0.00 | 4.05 |

100DMF-vs-Veh, 12 h

| | | | | | | |
|---|---|---|---|---|---|---|
| 1422748_PM_at | zinc finger E-box binding | Zeb2 | 24136 | −1.66 | 0.00 | 1.22 |
| | homeobox 2 | | | | | |
| 1438236_PM_at | nuclear factor I/A | Nfia | 18027 | −1.53 | 0.00 | 2.14 |

100MMF-vs-Veh, 2 h

| | | | | | | |
|---|---|---|---|---|---|---|
| 1421616_PM_at | glutamate receptor, ionotropic, | Grin2a | 14811 | −1.78 | 0.00 | 1.25 |
| | NMDA2A (epsilon 1) | | | | | |
| 1427125_PM_s_at | leucine rich repeat containing 41 | Lrrc41 | 230654 | −1.54 | 0.00 | 1.13 |
| 1428320_PM_at | KDM3B lysine (K)-specific | Kdm3b | 277250 | −1.56 | 0.00 | 2.17 |
| | demethylase 3B | | | | | |
| 1442867_PM_at | — | — | — | −1.57 | 0.00 | 1.07 |
| 1448458_PM_at | topoisomerase (DNA) II beta | Top2b | 21974 | −1.56 | 0.00 | 0.04 |
| 1449616_PM_s_at | golgi autoantigen, golgin subfamily | Golga3 | 269682 | −1.63 | 0.00 | 0.65 |
| | a, 3 | | | | | |

100MMF-vs-Veh, 7 h

| | | | | | | |
|---|---|---|---|---|---|---|
| 1419435_PM_at | aldehyde oxidase 1 | Aox1 | 11761 | 1.95 | 0.00 | 6.56 |

100MMF-vs-Veh, 12 h

| | | | | | | |
|---|---|---|---|---|---|---|
| 1428676_PM_at | transmembrane serine protease 6 | Tmprss6 | 71753 | 1.68 | 0.00 | 1.03 |
| 1435272_PM_at | inositol 1,4,5-trisphosphate 3-kinase B | Itpkb | 320404 | −1.56 | 0.00 | 0.71 |
| 1437760_PM_at | UDP-N-acetyl-alpha-D- | Galnt12 | 230145 | 1.50 | 0.00 | 0.63 |
| | galactosamine:polypeptide N- | | | | | |
| | acetylgalactosaminyltransferase 12 | | | | | |
| 1437785_PM_at | a disintegrin-like and metallopeptidase | Adamts9 | 101401 | −2.58 | 0.00 | 2.42 |
| | (reprolysin type) with thrombospondin type 1 | | | | | |
| | motif, 9 | | | | | |
| 1448807_PM_at | histamine receptor H3 | Hrh3 | 99296 | 1.59 | 0.00 | 0.28 |
| 1455123_PM_at | suppression of tumorigenicity 18 | St18 | 240690 | −1.69 | 0.00 | 0.21 |
| 1457257_PM_x_at | poliovirus receptor-related 3 | Pvrl3 | 58998 | −1.80 | 0.00 | 1.69 |

100DMF-vs-100MMF, 2 h
None
100DMF-vs-100MMF, 7 h
None
100DMF-vs-100MMF, 12 h
None
JEJUNUM
100DMF-vs-Veh, 2 h

| | | | | | | |
|---|---|---|---|---|---|---|
| 1416368_PM_at | glutathione S-transferase, | Gsta4 | 14860 | 1.72 | 0.00 | 1.41 |
| | alpha 4 | | | | | |
| 1416416_PM_x_at | glutathione S-transferase, | Gstm1 | 14862 | 2.30 | 0.00 | 5.29 |
| | mu 1 | | | | | |
| 1416552_PM_at | developmental | Dppa5a | 434423 | 3.07 | 0.00 | 12.50 |
| | pluripotency associated | | | | | |
| | 5A | | | | | |
| 1417061_PM_at | solute carrier family 40 | Slc40a1 | 53945 | 2.33 | 0.00 | 0.59 |
| | (iron-regulated | | | | | |
| | transporter), member 1 | | | | | |
| 1418320_PM_at | protease, serine, 8 | Prss8 | 76560 | 2.60 | 0.00 | 11.32 |
| | (prostasin) | | | | | |
| 1418672_PM_at | aldo-keto reductase family | Akr1c13 | 27384 | 1.62 | 0.00 | 3.95 |
| | 1, member C13 | | | | | |
| 1419030_PM_at | ERO1-like (S. cerevisiae) | Ero1l | 50527 | 1.92 | 0.00 | 16.12 |
| 1419431_PM_at | epiregulin | Ereg | 13874 | 2.50 | 0.00 | 11.82 |
| 1419510_PM_at | carboxylesterase 1E | Ces1e | 13897 | 1.85 | 0.00 | 3.49 |
| 1419622_PM_at | UDP | Ugt2b5 | 22238 | 3.40 | 0.00 | 5.25 |
| | glucuronosyltransferase 2 | | | | | |
| | family, polypeptide B5 | | | | | |
| 1421040_PM_a_at | glutathione S-transferase, | Gsta2 | 14858 | 3.82 | 0.00 | 2.22 |
| | alpha 2 (Yc2) | | | | | |
| 1421134_PM_at | amphiregulin | Areg | 11839 | 3.58 | 0.00 | 6.36 |
| 1421816_PM_at | glutathione reductase | Gsr | 14782 | 2.23 | 0.00 | 1.51 |
| 1423436_PM_at | glutathione S-transferase, | Gsta3 | 14859 | 3.23 | 0.00 | 3.00 |
| | alpha 3 | | | | | |
| 1423437_PM_at | glutathione S-transferase, | Gsta3 | 14859 | 4.03 | 0.00 | 6.21 |
| | alpha 3 | | | | | |
| 1423627_PM_at | NAD(P)H dehydrogenase, | Nqo1 | 18104 | 2.16 | 0.00 | 12.19 |
| | quinone 1 | | | | | |
| 1424022_PM_at | oxidative stress induced | Osgin1 | 71839 | 2.08 | 0.00 | 12.44 |
| | growth inhibitor 1 | | | | | |
| 1424266_PM_s_at | carboxylesterase 1F | Ces1f | 234564 | 2.48 | 0.00 | 2.42 |
| 1424296_PM_at | glutamate-cysteine ligase, | Gclc | 14629 | 4.25 | 0.00 | 17.03 |
| | catalytic subunit | | | | | |
| 1425239_PM_at | SET domain containing 4 | Setd4 | 224440 | 2.09 | 0.00 | 10.43 |
| 1425351_PM_at | sulfiredoxin 1 homolog | Srxn1 | 76650 | 1.71 | 0.00 | 11.67 |
| | (S. cerevisiae) | | | | | |
| 1425627_PM_x_at | glutathione S-transferase, | Gstm1 | 14862 | 2.57 | 0.00 | 3.59 |
| | mu 1 | | | | | |
| 1426501_PM_a_at | TRAF-interacting protein | Tifa | 211550 | −2.43 | 0.00 | 0.49 |
| | with forkhead-associated | | | | | |
| | domain | | | | | |
| 1427473_PM_at | glutathione S-transferase, | Gstm3 | 14864 | 2.37 | 0.00 | 6.53 |
| | mu 3 | | | | | |
| 1427474_PM_s_at | glutathione S-transferase, | Gstm3 | 14864 | 1.56 | 0.00 | 6.67 |
| | mu 3 | | | | | |
| 1427912_PM_at | carbonyl reductase 3 | Cbr3 | 109857 | 54.72 | 0.00 | 25.30 |
| 1428805_PM_at | solute carrier family 35, | Slc35e3 | 215436 | 1.53 | 0.00 | 2.85 |
| | member E3 | | | | | |
| 1428834_PM_at | dual specificity | Dusp4 | 319520 | 1.51 | 0.00 | 0.92 |
| | phosphatase 4 | | | | | |
| 1429950_PM_at | unc-5 homolog C (C. elegans)- | Unc5cl | 76589 | −1.53 | 0.00 | 3.15 |
| | like | | | | | |
| 1430135_PM_at | deoxyribonuclease II | Dnase2a | 13423 | 1.77 | 0.00 | 4.55 |
| | alpha | | | | | |
| 1433763_PM_at | ectonucleoside | Entpd5 | 12499 | 1.53 | 0.00 | 8.35 |
| | triphosphate | | | | | |
| | diphosphohydrolase 5 | | | | | |
| 1434054_PM_at | v-maf | Mafg | 17134 | 1.96 | 0.00 | 17.49 |
| | musculoaponeurotic | | | | | |
| | fibrosarcoma oncogene | | | | | |
| | family, protein G (avian) | | | | | |
| 1435405_PM_at | SET domain containing 4 | Setd4 | 224440 | 1.91 | 0.00 | 9.69 |
| 1436600_PM_at | TOX high mobility group | Tox3 | 244579 | 1.70 | 0.00 | 3.80 |
| | box family member 3 | | | | | |
| 1439624_PM_at | UDP | Ugt2b35 | 243085 | 3.22 | 0.00 | 15.01 |
| | glucuronosyltransferase 2 | | | | | |
| | family, polypeptide B35 | | | | | |
| 1440230_PM_at | tsukushin | Tsku | 244152 | 1.61 | 0.00 | 3.15 |
| 1441046_PM_at | — | — | — | 1.54 | 0.00 | 19.30 |
| 1441604_PM_at | Esterase | Esd | 13885 | 1.71 | 0.00 | 11.56 |
| | D/formylglutathione | | | | | |
| | hydrolase | | | | | |
| 1441931_PM_x_at | glutathione synthetase | Gss | 14854 | 1.95 | 0.00 | 7.94 |
| 1442923_PM_at | PTK6 protein tyrosine | Ptk6 | 20459 | −2.42 | 0.00 | 0.38 |
| | kinase 6 | | | | | |
| 1443118_PM_at | — | — | — | 1.91 | 0.00 | 7.70 |
| 1443159_PM_at | RIKEN cDNA | 9130221J17Rik | 319693 | 2.30 | 0.00 | 19.68 |
| | 9130221J17 gene | | | | | |
| 1444265_PM_at | — | — | — | 1.75 | 0.00 | 0.35 |
| 1445980_PM_at | — | — | — | 2.92 | 0.00 | 11.73 |
| 1446045_PM_at | — | — | — | 1.52 | 0.00 | 0.88 |
| 1446542_PM_at | acyl-CoA synthetase | Acss2 | 60525 | 1.68 | 0.00 | 7.47 |
| | short-chain family | | | | | |
| | member 2 | | | | | |
| 1447411_PM_at | — | — | — | 2.88 | 0.00 | 8.87 |
| 1448273_PM_at | glutathione synthetase | Gss | 14854 | 1.67 | 0.00 | 4.28 |
| 1448330_PM_at | glutathione S-transferase, | Gstm1 | 14862 | 2.47 | 0.00 | 4.91 |
| | mu 1 | | | | | |
| 1448894_PM_at | aldo-keto reductase family | Akr1b8 | 14187 | 7.35 | 0.00 | 12.36 |
| | 1, member B8 | | | | | |
| 1448916_PM_at | v-maf | Mafg | 17134 | 2.20 | 0.00 | 17.19 |
| | musculoaponeurotic | | | | | |
| | fibrosarcoma oncogene | | | | | |
| | family, protein G (avian) | | | | | |
| 1449324_PM_at | ERO1-like (S. cerevisiae) | Ero1l | 50527 | 1.50 | 0.00 | 6.53 |
| 1449486_PM_at | carboxylesterase 1G | Ces1g | 12623 | 4.06 | 0.00 | 3.47 |
| 1453421_PM_at | serine racemase | Srr | 27364 | 1.99 | 0.00 | 5.27 |
| 1455454_PM_at | aldo-keto reductase family | Akr1c19 | 432720 | 2.45 | 0.00 | 8.49 |
| | 1, member C19 | | | | | |
| 1455595_PM_at | UDP | Ugt2b36 | 231396 | 2.07 | 0.00 | 2.47 |
| | glucuronosyltransferase 2 | | | | | |
| | family, polypeptide B36 | | | | | |
| 1455959_PM_s_at | glutamate-cysteine ligase, | Gclc | 14629 | 3.13 | 0.00 | 17.65 |
| | catalytic subunit | | | | | |
| 1456611_PM_at | family with sequence | Fam13a | 58909 | 1.54 | 0.00 | 1.13 |
| | similarity 13, member A | | | | | |
| 1457279_PM_at | hypothetical | LOC100504040 | 100504040 | 2.18 | 0.00 | 24.32 |
| | LOC100504040 | | | | | |
| 1459091_PM_at | — | — | — | 1.98 | 0.00 | 5.68 |
| 1460196_PM_at | carbonyl reductase 1 | Cbr1 | 12408 | 1.55 | 0.00 | 9.97 |
| 1460373_PM_a_at | SET domain containing 4 | Setd4 | 224440 | 1.91 | 0.00 | 12.43 |

100DMF-vs-Veh, 7 h

| | | | | | | |
|---|---|---|---|---|---|---|
| 1416368_PM_at | glutathione S-transferase, | Gsta4 | 14860 | 2.09 | 0.00 | 9.86 |
| | alpha 4 | | | | | |
| 1416411_PM_at | glutathione S-transferase, mu 2 | Gstm2 | 14863 | 1.58 | 0.00 | 0.92 |
| 1416416_PM_x_at | glutathione S-transferase, mu 1 | Gstm1 | 14862 | 3.19 | 0.00 | 17.39 |
| 1418672_PM_at | aldo-keto reductase family 1, | Akr1c13 | 27384 | 1.61 | 0.00 | 5.33 |
| | member C13 | | | | | |
| 1419510_PM_at | carboxylesterase 1E | Ces1e | 13897 | 2.10 | 0.00 | 9.86 |
| 1419622_PM_at | UDP glucuronosyltransferase 2 | Ugt2b5 | 22238 | 3.42 | 0.00 | 7.21 |
| | family, polypeptide B5 | | | | | |
| 1421040_PM_a_at | glutathione S-transferase, | Gsta2 | 14858 | 5.81 | 0.00 | 10.24 |
| | alpha 2 (Yc2) | | | | | |
| 1421041_PM_s_at | glutathione S-transferase, | Gsta2 | 14858 | 1.72 | 0.00 | 9.81 |
| | alpha 2 (Yc2) | | | | | |
| 1421816_PM_at | glutathione reductase | Gsr | 14782 | 2.83 | 0.00 | 8.73 |
| 1422072_PM_a_at | glutathione S-transferase, mu 6 | Gstm6 | 14867 | 1.94 | 0.00 | 0.31 |
| 1422327_PM_s_at | glucose-6-phosphate | G6pd2 | 14380 | 1.82 | 0.00 | 6.24 |
| | dehydrogenase 2 | | | | | |
| 1422757_PM_at | solute carrier family 5 (neutral | Slc5a4b | 64454 | 1.67 | 0.00 | 10.87 |
| | amino acid transporters, | | | | | |
| | system A), member 4b | | | | | |
| 1423436_PM_at | glutathione S-transferase, | Gsta3 | 14859 | 4.27 | 0.00 | 9.78 |
| | alpha 3 | | | | | |
| 1423437_PM_at | glutathione S-transferase, | Gsta3 | 14859 | 3.89 | 0.00 | 7.49 |
| | alpha 3 | | | | | |
| 1423627_PM_at | NAD(P)H dehydrogenase, | Nqo1 | 18104 | 2.31 | 0.00 | 17.89 |
| | quinone 1 | | | | | |
| 1424266_PM_s_at | carboxylesterase 1F | Ces1f | 234564 | 3.58 | 0.00 | 12.60 |
| 1424296_PM_at | glutamate-cysteine ligase, | Gclc | 14629 | 2.06 | 0.00 | 1.34 |
| | catalytic subunit | | | | | |
| 1424487_PM_x_at | thioredoxin reductase 1 | Txnrd1 | 50493 | 1.60 | 0.00 | 0.38 |
| 1424835_PM_at | glutathione S-transferase, mu 4 | Gstm4 | 14865 | 3.83 | 0.00 | 10.20 |
| 1425239_PM_at | SET domain containing 4 | Setd4 | 224440 | 1.90 | 0.00 | 8.73 |
| 1425627_PM_x_at | glutathione S-transferase, mu 1 | Gstm1 | 14862 | 3.45 | 0.00 | 12.53 |
| 1426215_PM_at | dopa decarboxylase | Ddc | 13195 | 1.74 | 0.00 | 6.22 |
| 1427473_PM_at | glutathione S-transferase, mu 3 | Gstm3 | 14864 | 2.82 | 0.00 | 14.02 |
| 1427474_PM_s_at | glutathione S-transferase, mu 3 | Gstm3 | 14864 | 1.79 | 0.00 | 17.29 |
| 1427912_PM_at | carbonyl reductase 3 | Cbr3 | 109857 | 22.39 | 0.00 | 18.25 |
| 1428988_PM_at | ATP-binding cassette, sub- | Abcc3 | 76408 | 1.88 | 0.00 | 7.24 |
| | family C (CFTR/MRP), | | | | | |
| | member 3 | | | | | |
| 1429001_PM_at | pirin | Pir | 69656 | 3.79 | 0.00 | 19.79 |
| 1431672_PM_at | RIKEN cDNA 9430069I07 | 9430069I07Rik | 77358 | 2.76 | 0.00 | 6.45 |
| | gene | | | | | |
| 1435405_PM_at | SET domain containing 4 | Setd4 | 224440 | 1.50 | 0.00 | 1.46 |
| 1437662_PM_at | acyl-CoA synthetase medium- | Acsm5 | 272428 | 1.60 | 0.00 | 1.12 |
| | chain family member 5 | | | | | |
| 1439624_PM_at | UDP glucuronosyltransferase 2 | Ugt2b35 | 243085 | 2.66 | 0.00 | 11.87 |
| | family, polypeptide B35 | | | | | |
| 1441931_PM_x_at | glutathione synthetase | Gss | 14854 | 1.89 | 0.00 | 8.72 |
| 1446542_PM_at | acyl-CoA synthetase short- | Acss2 | 60525 | 1.71 | 0.00 | 10.70 |
| | chain family member 2 | | | | | |
| 1448273_PM_at | glutathione synthetase | Gss | 14854 | 1.97 | 0.00 | 13.77 |
| 1448330_PM_at | glutathione S-transferase, mu 1 | Gstm1 | 14862 | 3.40 | 0.00 | 15.64 |
| 1448354_PM_at | glucose-6-phosphate | G6pdx | 14381 | 1.54 | 0.00 | 12.11 |
| | dehydrogenase X-linked | | | | | |
| 1448894_PM_at | aldo-keto reductase family 1, | Akr1b8 | 14187 | 8.53 | 0.00 | 17.62 |
| | member B8 | | | | | |
| 1449486_PM_at | carboxylesterase 1G | Ces1g | 12623 | 4.67 | 0.00 | 7.34 |
| 1451814_PM_a_at | HIV-1 tat interactive protein 2, | Htatip2 | 53415 | 1.51 | 0.00 | 3.02 |
| | homolog (human) | | | | | |
| 1455454_PM_at | aldo-keto reductase family 1, | Akr1c19 | 432720 | 2.07 | 0.00 | 5.27 |
| | member C19 | | | | | |
| 1455595_PM_at | UDP glucuronosyltransferase 2 | Ugt2b36 | 231396 | 2.23 | 0.00 | 6.13 |
| | family, polypeptide B36 | | | | | |
| 1455959_PM_s_at | glutamate-cysteine ligase, | Gclc | 14629 | 1.88 | 0.00 | 3.38 |
| | catalytic subunit | | | | | |
| 1460196_PM_at | carbonyl reductase 1 | Cbr1 | 12408 | 1.65 | 0.00 | 16.88 |
| 1460373_PM_a_at | SET domain containing 4 | Setd4 | 224440 | 1.50 | 0.00 | 2.85 |

100DMF-vs-Veh, 12 h

| | | | | | | |
|---|---|---|---|---|---|---|
| 1416368_PM_at | glutathione S-transferase, alpha 4 | Gsta4 | 14860 | 2.58 | 0.00 | 18.11 |
| 1416411_PM_at | glutathione S-transferase, mu 2 | Gstm2 | 14863 | 2.04 | 0.00 | 10.57 |
| 1416416_PM_x_at | glutathione S-transferase, mu 1 | Gstm1 | 14862 | 3.56 | 0.00 | 20.84 |
| 1418601_PM_at | aldehyde dehydrogenase family 1, | Aldh1a7 | 26358 | 1.65 | 0.00 | 8.43 |
| | subfamily A7 | | | | | |
| 1418672_PM_at | aldo-keto reductase family 1, member | Akr1c13 | 27384 | 1.96 | 0.00 | 14.88 |
| | C13 | | | | | |
| 1419510_PM_at | carboxylesterase 1E | Ces1e | 13897 | 2.70 | 0.00 | 19.65 |
| 1419622_PM_at | UDP glucuronosyltransferase 2 family, | Ugt2b5 | 22238 | 4.92 | 0.00 | 14.87 |
| | polypeptide B5 | | | | | |
| 1421040_PM_a_at | glutathione S-transferase, alpha 2 | Gsta2 | 14858 | 5.65 | 0.00 | 9.78 |
| | (Yc2) | | | | | |
| 1421041_PM_s_at | glutathione S-transferase, alpha 2 | Gsta2 | 14858 | 2.04 | 0.00 | 18.90 |
| | (Yc2) | | | | | |
| 1421816_PM_at | glutathione reductase | Gsr | 14782 | 2.20 | 0.00 | 2.62 |
| 1422072_PM_a_at | glutathione S-transferase, mu 6 | Gstm6 | 14867 | 1.95 | 0.00 | 0.40 |
| 1422327_PM_s_at | glucose-6-phosphate dehydrogenase 2 | G6pd2 | 14380 | 1.74 | 0.00 | 4.49 |
| 1422757_PM_at | solute carrier family 5 (neutral amino | Slc5a4b | 64454 | 2.21 | 0.00 | 27.56 |
| | acid transporters, system A), member | | | | | |
| | 4b | | | | | |
| 1423436_PM_at | glutathione S-transferase, alpha 3 | Gsta3 | 14859 | 10.36 | 0.00 | 27.54 |
| 1423437_PM_at | glutathione S-transferase, alpha 3 | Gsta3 | 14859 | 7.86 | 0.00 | 21.21 |
| 1423627_PM_at | NAD(P)H dehydrogenase, quinone 1 | Nqo1 | 18104 | 2.30 | 0.00 | 17.71 |
| 1424266_PM_s_at | carboxylesterase 1F | Ces1f | 234564 | 6.17 | 0.00 | 26.23 |
| 1424835_PM_at | glutathione S-transferase, mu 4 | Gstm4 | 14865 | 5.03 | 0.00 | 16.14 |
| 1425239_PM_at | SET domain containing 4 | Setd4 | 224440 | 1.66 | 0.00 | 3.46 |
| 1425627_PM_x_at | glutathione S-transferase, mu 1 | Gstm1 | 14862 | 4.63 | 0.00 | 20.08 |
| 1427473_PM_at | glutathione S-transferase, mu 3 | Gstm3 | 14864 | 2.03 | 0.00 | 3.91 |
| 1427474_PM_s_at | glutathione S-transferase, mu 3 | Gstm3 | 14864 | 1.57 | 0.00 | 8.96 |
| 1427912_PM_at | carbonyl reductase 3 | Cbr3 | 109857 | 25.95 | 0.00 | 20.05 |
| 1428988_PM_at | ATP-binding cassette, sub-family C | Abcc3 | 76408 | 2.02 | 0.00 | 9.98 |
| | (CFTR/MRP), member 3 | | | | | |
| 1429001_PM_at | pirin | Pir | 69656 | 3.79 | 0.00 | 19.79 |
| 1437662_PM_at | acyl-CoA synthetase medium-chain | Acsm5 | 272428 | 1.86 | 0.00 | 6.62 |
| | family member 5 | | | | | |
| 1439624_PM_at | UDP glucuronosyltransferase 2 family, | Ugt2b35 | 243085 | 2.37 | 0.00 | 8.22 |
| | polypeptide B35 | | | | | |
| 1441931_PM_x_at | glutathione synthetase | Gss | 14854 | 1.53 | 0.00 | 0.45 |
| 1448330_PM_at | glutathione S-transferase, mu 1 | Gstm1 | 14862 | 4.15 | 0.00 | 21.42 |
| 1448894_PM_at | aldo-keto reductase family 1, member | Akr1b8 | 14187 | 17.57 | 0.00 | 30.06 |
| | B8 | | | | | |
| 1449279_PM_at | glutathione peroxidase 2 | Gpx2 | 14776 | 1.67 | 0.00 | 16.76 |
| 1449486_PM_at | carboxylesterase 1G | Ces1g | 12623 | 7.40 | 0.00 | 15.08 |
| 1450455_PM_s_at | aldo-keto reductase family 1, member | Akr1c12 | 27384 | 1.72 | 0.00 | 17.47 |
| | C12 | | | | | |
| 1451386_PM_at | biliverdin reductase B (flavin reductase | Blvrb | 233016 | 1.53 | 0.00 | 6.13 |
| | (NADPH)) | | | | | |
| 1455454_PM_at | aldo-keto reductase family 1, member | Akr1c19 | 432720 | 3.20 | 0.00 | 19.61 |
| | C19 | | | | | |
| 1455595_PM_at | UDP glucuronosyltransferase 2 family, | Ugt2b36 | 231396 | 2.94 | 0.00 | 14.55 |
| | polypeptide B36 | | | | | |
| 1460373_PM_a_at | SET domain containing 4 | Setd4 | 224440 | 1.52 | 0.00 | 3.56 |

100MMF-vs-Veh, 2 h

| | | | | | | |
|---|---|---|---|---|---|---|
| 1416368_PM_at | glutathione S-transferase, alpha 4 | Gsta4 | 14860 | 1.73 | 0.00 | 1.54 |
| 1416416_PM_x_at | glutathione S-transferase, mu 1 | Gstm1 | 14862 | 2.38 | 0.00 | 6.23 |
| 1416418_PM_at | gamma-aminobutyric acid | Gabarapl1 | 57436 | 1.52 | 0.00 | 2.11 |
| | (GABA) A receptor-associated | | | | | |
| | protein-like 1 | | | | | |
| 1416552_PM_at | developmental pluripotency | Dppa5a | 434423 | 2.64 | 0.00 | 8.36 |
| | associated 5A | | | | | |
| 1417061_PM_at | solute carrier family 40 (iron- | Slc40a1 | 53945 | 2.73 | 0.00 | 3.48 |
| | regulated transporter), member 1 | | | | | |
| 1418320_PM_at | protease, serine, 8 (prostasin) | Prss8 | 76560 | 2.41 | 0.00 | 8.91 |
| 1419030_PM_at | ERO1-like (S. cerevisiae) | Ero1l | 50527 | 1.90 | 0.00 | 15.61 |
| 1419431_PM_at | epiregulin | Ereg | 13874 | 2.00 | 0.00 | 4.69 |
| 1419622_PM_at | UDP glucuronosyltransferase 2 | Ugt2b5 | 22238 | 3.13 | 0.00 | 3.81 |
| | family, polypeptide B5 | | | | | |
| 1421134_PM_at | amphiregulin | Areg | 11839 | 2.71 | 0.00 | 1.49 |
| 1421816_PM_at | glutathione reductase | Gsr | 14782 | 2.19 | 0.00 | 1.18 |
| 1422327_PM_s_at | glucose-6-phosphate | G6pd2 | 14380 | 1.74 | 0.00 | 3.00 |
| | dehydrogenase 2 | | | | | |
| 1422645_PM_at | hemochromatosis | Hfe | 15216 | 1.73 | 0.00 | 0.44 |
| 1423306_PM_at | RIKEN cDNA 2010002N04 gene | 2010002N04Rik | 106878 | 1.57 | 0.00 | 8.95 |
| 1423436_PM_at | glutathione S-transferase, alpha 3 | Gsta3 | 14859 | 3.23 | 0.00 | 3.00 |
| 1423437_PM_at | glutathione S-transferase, alpha 3 | Gsta3 | 14859 | 3.64 | 0.00 | 4.53 |
| 1423627_PM_at | NAD(P)H dehydrogenase, | Nqo1 | 18104 | 2.16 | 0.00 | 12.15 |
| | quinone 1 | | | | | |
| 1423635_PM_at | bone morphogenetic protein 2 | Bmp2 | 12156 | −1.55 | 0.00 | 0.94 |
| 1424022_PM_at | oxidative stress induced growth | Osgin1 | 71839 | 1.98 | 0.00 | 10.25 |
| | inhibitor 1 | | | | | |
| 1424181_PM_at | septin 6 | 6-Sep | 56526 | 1.56 | 0.00 | 0.83 |
| 1424266_PM_s_at | carboxylesterase 1F | Ces1f | 234564 | 2.71 | 0.00 | 4.18 |
| 1424296_PM_at | glutamate-cysteine ligase, | Gclc | 14629 | 3.99 | 0.00 | 15.44 |
| | catalytic subunit | | | | | |
| 1425239_PM_at | SET domain containing 4 | Setd4 | 224440 | 2.11 | 0.00 | 10.76 |
| 1425351_PM_at | sulfiredoxin 1 homolog (S. cerevisiae) | Srxn1 | 76650 | 1.77 | 0.00 | 13.61 |
| 1425627_PM_x_at | glutathione S-transferase, mu 1 | Gstm1 | 14862 | 2.70 | 0.00 | 4.63 |
| 1426599_PM_a_at | solute carrier family 2 (facilitated | Slc2a1 | 20525 | 1.53 | 0.00 | 7.97 |
| | glucose transporter), member 1 | | | | | |
| 1426600_PM_at | solute carrier family 2 (facilitated | Slc2a1 | 20525 | 1.68 | 0.00 | 8.19 |
| | glucose transporter), member 1 | | | | | |
| 1427473_PM_at | glutathione S-transferase, mu 3 | Gstm3 | 14864 | 2.51 | 0.00 | 8.06 |
| 1427474_PM_s_at | glutathione S-transferase, mu 3 | Gstm3 | 14864 | 1.57 | 0.00 | 7.05 |
| 1427912_PM_at | carbonyl reductase 3 | Cbr3 | 109857 | 55.49 | 0.00 | 25.46 |
| 1430135_PM_at | deoxyribonuclease II alpha | Dnase2a | 13423 | 1.79 | 0.00 | 4.90 |
| 1433699_PM_at | tumor necrosis factor, alpha- | Tnfaip3 | 21929 | −1.67 | 0.00 | 0.32 |
| | induced protein 3 | | | | | |
| 1434054_PM_at | v-maf musculoaponeurotic | Mafg | 17134 | 1.95 | 0.00 | 17.17 |
| | fibrosarcoma oncogene family, | | | | | |
| | protein G (avian) | | | | | |
| 1434169_PM_at | RIKEN cDNA 9030409G11 gene | 9030409G11Rik | 71529 | 1.52 | 0.00 | 3.39 |
| 1435405_PM_at | SET domain containing 4 | Setd4 | 224440 | 1.93 | 0.00 | 10.17 |
| 1436600_PM_at | TOX high mobility group box | Tox3 | 244579 | 1.81 | 0.00 | 6.32 |
| | family member 3 | | | | | |
| 1436605_PM_at | transketolase | Tkt | 21881 | 1.73 | 0.00 | 8.55 |
| 1438953_PM_at | c-fos induced growth factor | Figf | 14205 | 1.63 | 0.00 | 0.69 |
| 1439624_PM_at | UDP glucuronosyltransferase 2 | Ugt2b35 | 243085 | 3.08 | 0.00 | 13.73 |
| | family, polypeptide B35 | | | | | |
| 1440882_PM_at | low density lipoprotein receptor- | Lrp8 | 16975 | 2.23 | 0.00 | 7.73 |
| | related protein 8, apolipoprotein e | | | | | |
| | receptor | | | | | |
| 1441604_PM_at | Esterase D/formylglutathione | Esd | 13885 | 1.52 | 0.00 | 5.03 |
| | hydrolase | | | | | |
| 1441931_PM_x_at | glutathione synthetase | Gss | 14854 | 1.92 | 0.00 | 7.30 |
| 1443118_PM_at | — | — | — | 1.69 | 0.00 | 3.21 |
| 1443159_PM_at | RIKEN cDNA 9130221J17 gene | 9130221J17Rik | 319693 | 2.36 | 0.00 | 20.89 |
| 1444265_PM_at | — | — | — | 1.93 | 0.00 | 3.06 |
| 1445980_PM_at | — | — | — | 2.43 | 0.00 | 6.59 |
| 1446045_PM_at | — | — | — | 1.69 | 0.00 | 5.02 |
| 1446542_PM_at | acyl-CoA synthetase short-chain | Acss2 | 60525 | 1.84 | 0.00 | 12.15 |
| | family member 2 | | | | | |
| 1447411_PM_at | — | — | — | 2.81 | 0.00 | 8.18 |
| 1448273_PM_at | glutathione synthetase | Gss | 14854 | 1.69 | 0.00 | 4.79 |
| 1448330_PM_at | glutathione S-transferase, mu 1 | Gstm1 | 14862 | 2.47 | 0.00 | 4.96 |
| 1448894_PM_at | aldo-keto reductase family 1, | Akr1b8 | 14187 | 8.24 | 0.00 | 14.17 |
| | member B8 | | | | | |
| 1448916_PM_at | v-maf musculoaponeurotic | Mafg | 17134 | 2.01 | 0.00 | 13.16 |
| | fibrosarcoma oncogene family, | | | | | |
| | protein G (avian) | | | | | |
| 1449078_PM_at | ST3 beta-galactoside alpha-2,3- | St3gal6 | 54613 | 1.52 | 0.00 | 3.97 |
| | sialyltransferase 6 | | | | | |
| 1449486_PM_at | carboxylesterase 1G | Ces1g | 12623 | 3.68 | 0.00 | 2.16 |
| 1450165_PM_at | schlafen 2 | Slfn2 | 20556 | −1.82 | 0.00 | 0.53 |
| 1450410_PM_a_at | solute carrier family 48 (heme | Slc48a1 | 67739 | 1.62 | 0.00 | 4.04 |
| | transporter), member 1 | | | | | |
| 1451386_PM_at | biliverdin reductase B (flavin | Blvrb | 233016 | 1.57 | 0.00 | 5.61 |
| | reductase (NADPH)) | | | | | |
| 1451814_PM_a_at | HIV-1 tat interactive protein 2, | Htatip2 | 53415 | 1.53 | 0.00 | 2.07 |
| | homolog (human) | | | | | |
| 1452834_PM_at | proline rich 5 like | Prr5l | 72446 | −1.64 | 0.00 | 5.44 |
| 1452837_PM_at | lipin 2 | Lpin2 | 64898 | 1.61 | 0.00 | 0.31 |
| 1455454_PM_at | aldo-keto reductase family 1, | Akr1c19 | 432720 | 2.33 | 0.00 | 7.03 |
| | member C19 | | | | | |
| 1455959_PM_s_at | glutamate-cysteine ligase, | Gclc | 14629 | 3.06 | 0.00 | 16.87 |
| | catalytic subunit | | | | | |
| 1457279_PM_at | hypothetical LOC100504040 | LOC100504040 | 100504040 | 2.24 | 0.00 | 25.82 |
| 1459091_PM_at | — | — | — | 2.02 | 0.00 | 6.40 |
| 1460196_PM_at | carbonyl reductase 1 | Cbr1 | 12408 | 1.53 | 0.00 | 8.80 |
| 1460373_PM_a_at | SET domain containing 4 | Setd4 | 224440 | 1.87 | 0.00 | 11.36 |

100MMF-vs-Veh, 7 h

| | | | | | | |
|---|---|---|---|---|---|---|
| 1416368_PM_at | glutathione S-transferase, alpha 4 | Gsta4 | 14860 | 2.52 | 0.00 | 17.09 |
| 1416411_PM_at | glutathione S-transferase, mu 2 | Gstm2 | 14863 | 2.09 | 0.00 | 11.48 |
| 1416416_PM_x_at | glutathione S-transferase, mu 1 | Gstm1 | 14862 | 3.95 | 0.00 | 24.15 |
| 1416552_PM_at | developmental pluripotency | Dppa5a | 434423 | 1.88 | 0.00 | 1.05 |
| | associated 5A | | | | | |
| 1416632_PM_at | malic enzyme 1, NADP(+)- | Me1 | 17436 | 2.01 | 0.00 | 1.18 |
| | dependent, cytosolic | | | | | |
| 1418320_PM_at | protease, serine, 8 (prostasin) | Prss8 | 76560 | 1.75 | 0.00 | 1.06 |
| 1418580_PM_at | receptor transporter protein 4 | Rtp4 | 67775 | −1.61 | 0.00 | 1.72 |
| 1418601_PM_at | aldehyde dehydrogenase family 1, | Aldh1a7 | 26358 | 1.86 | 0.00 | 14.89 |
| | subfamily A7 | | | | | |
| 1418672_PM_at | aldo-keto reductase family 1, | Akr1c13 | 27384 | 1.86 | 0.00 | 12.22 |
| | member C13 | | | | | |
| 1419072_PM_at | glutathione S-transferase, mu 7 | Gstm7 | 68312 | 1.97 | 0.00 | 3.79 |
| 1419442_PM_at | matrilin 2 | Matn2 | 17181 | 1.59 | 0.00 | 17.50 |
| 1419510_PM_at | carboxylesterase 1E | Ces1e | 13897 | 2.44 | 0.00 | 15.68 |
| 1419622_PM_at | UDP glucuronosyltransferase 2 | Ugt2b5 | 22238 | 4.50 | 0.00 | 12.97 |
| | family, polypeptide B5 | | | | | |
| 1421009_PM_at | radical S-adenosyl methionine | Rsad2 | 58185 | −1.84 | 0.00 | 1.99 |
| | domain containing 2 | | | | | |
| 1421040_PM_a_at | glutathione S-transferase, alpha 2 | Gsta2 | 14858 | 9.84 | 0.00 | 19.06 |
| | (Yc2) | | | | | |
| 1421041_PM_s_at | glutathione S-transferase, alpha 2 | Gsta2 | 14858 | 1.90 | 0.00 | 14.89 |
| | (Yc2) | | | | | |
| 1421134_PM_at | amphiregulin | Areg | 11839 | 2.93 | 0.00 | 4.33 |
| 1421816_PM_at | glutathione reductase | Gsr | 14782 | 3.13 | 0.00 | 11.41 |
| 1422072_PM_a_at | glutathione S-transferase, mu 6 | Gstm6 | 14867 | 2.23 | 0.00 | 3.49 |
| 1422327_PM_s_at | glucose-6-phosphate dehydrogenase 2 | G6pd2 | 14380 | 2.06 | 0.00 | 11.30 |
| 1422757_PM_at | solute carrier family 5 (neutral | Slc5a4b | 64454 | 1.79 | 0.00 | 15.18 |
| | amino acid transporters, system A), | | | | | |
| | member 4b | | | | | |
| 1423436_PM_at | glutathione S-transferase, alpha 3 | Gsta3 | 14859 | 5.71 | 0.00 | 15.57 |
| 1423437_PM_at | glutathione S-transferase, alpha 3 | Gsta3 | 14859 | 5.75 | 0.00 | 15.03 |
| 1423627_PM_at | NAD(P)H dehydrogenase, quinone 1 | Nqo1 | 18104 | 2.63 | 0.00 | 23.56 |
| 1423706_PM_a_at | phosphogluconate dehydrogenase | Pgd | 110208 | 1.55 | 0.00 | 8.57 |
| 1424266_PM_s_at | carboxylesterase 1F | Ces1f | 234564 | 5.19 | 0.00 | 21.90 |
| 1424296_PM_at | glutamate-cysteine ligase, catalytic | Gclc | 14629 | 2.87 | 0.00 | 9.44 |
| | subunit | | | | | |
| 1424486_PM_a_at | thioredoxin reductase 1 | Txnrd1 | 50493 | 2.07 | 0.00 | 1.62 |
| 1424487_PM_x_at | thioredoxin reductase 1 | Txnrd1 | 50493 | 1.93 | 0.00 | 6.69 |
| 1424835_PM_at | glutathione S-transferase, mu 4 | Gstm4 | 14865 | 5.75 | 0.00 | 19.08 |
| 1425239_PM_at | SET domain containing 4 | Setd4 | 224440 | 2.27 | 0.00 | 16.40 |
| 1425627_PM_x_at | glutathione S-transferase, mu 1 | Gstm1 | 14862 | 4.80 | 0.00 | 21.02 |
| 1426215_PM_at | dopa decarboxylase | Ddc | 13195 | 2.03 | 0.00 | 13.12 |
| 1427473_PM_at | glutathione S-transferase, mu 3 | Gstm3 | 14864 | 3.46 | 0.00 | 20.60 |
| 1427474_PM_s_at | glutathione S-transferase, mu 3 | Gstm3 | 14864 | 1.99 | 0.00 | 24.06 |
| 1427912_PM_at | carbonyl reductase 3 | Cbr3 | 109857 | 40.98 | 0.00 | 25.60 |
| 1428805_PM_at | solute carrier family 35, member E3 | Slc35e3 | 215436 | 1.67 | 0.00 | 8.74 |
| 1428960_PM_at | enkurin, TRPC channel interacting | Enkur | 71233 | −1.56 | 0.00 | 0.02 |
| | protein | | | | | |
| 1428988_PM_at | ATP-binding cassette, sub-family C | Abcc3 | 76408 | 2.26 | 0.00 | 14.68 |
| | (CFTR/MRP), member 3 | | | | | |
| 1429001_PM_at | pirin | Pir | 69656 | 5.12 | 0.00 | 28.64 |
| 1431672_PM_at | RIKEN cDNA 9430069I07 gene | 9430069I07Rik | 77358 | 3.66 | 0.00 | 13.39 |
| 1435405_PM_at | SET domain containing 4 | Setd4 | 224440 | 1.76 | 0.00 | 8.33 |
| 1435529_PM_at | predicted gene 14446 | Gm14446 | 667373 | −1.65 | 0.00 | 2.84 |
| 1436058_PM_at | radical S-adenosyl methionine | Rsad2 | 58185 | −1.81 | 0.00 | 5.00 |
| | domain containing 2 | | | | | |
| 1437287_PM_at | RIKEN cDNA 1110020G09 gene | 1110020G09Rik | 68646 | 1.74 | 0.00 | 0.58 |
| 1437662_PM_at | acyl-CoA synthetase medium-chain | Acsm5 | 272428 | 1.71 | 0.00 | 3.63 |
| | family member 5 | | | | | |
| 1437960_PM_at | calpain 13 | Capn13 | 381122 | −1.75 | 0.00 | 0.21 |
| 1438488_PM_at | esterase D/formylglutathione | Esd | 13885 | 2.15 | 0.00 | 7.19 |
| | hydrolase | | | | | |
| 1439624_PM_at | UDP glucuronosyltransferase 2 | Ugt2b35 | 243085 | 3.31 | 0.00 | 18.81 |
| | family, polypeptide B35 | | | | | |
| 1441931_PM_x_at | glutathione synthetase | Gss | 14854 | 2.20 | 0.00 | 15.28 |
| 1443159_PM_at | RIKEN cDNA 9130221J17 gene | 9130221J17Rik | 319693 | 1.61 | 0.00 | 5.00 |
| 1446542_PM_at | acyl-CoA synthetase short-chain | Acss2 | 60525 | 2.06 | 0.00 | 20.99 |
| | family member 2 | | | | | |
| 1447411_PM_at | — | — | — | 1.97 | 0.00 | 1.12 |
| 1448273_PM_at | glutathione synthetase | Gss | 14854 | 2.25 | 0.00 | 20.28 |
| 1448330_PM_at | glutathione S-transferase, mu 1 | Gstm1 | 14862 | 4.23 | 0.00 | 21.98 |
| 1448354_PM_at | glucose-6-phosphate dehydrogenase | G6pdx | 14381 | 1.76 | 0.00 | 21.61 |
| | X-linked | | | | | |
| 1448894_PM_at | aldo-keto reductase family 1, | Akr1b8 | 14187 | 14.16 | 0.00 | 26.39 |
| | member B8 | | | | | |
| 1449279_PM_at | glutathione peroxidase 2 | Gpx2 | 14776 | 1.53 | 0.00 | 10.35 |
| 1449486_PM_at | carboxylesterase 1G | Ces1g | 12623 | 9.24 | 0.00 | 18.92 |
| 1450109_PM_s_at | ATP-binding cassette, sub-family C | Abcc2 | 12780 | 1.51 | 0.00 | 9.75 |
| | (CFTR/MRP), member 2 | | | | | |
| 1450455_PM_s_at | aldo-keto reductase family 1, | Akr1c12 | 27384 | 1.58 | 0.00 | 11.64 |
| | member C12 | | | | | |
| 1450783_PM_at | interferon-induced protein with | Ifit1 | 15957 | −1.95 | 0.00 | 5.79 |
| | tetratricopeptide repeats 1 | | | | | |
| 1451095_PM_at | asparagine synthetase | Asns | 27053 | 1.60 | 0.00 | 0.76 |
| 1451149_PM_at | phosphoglucomutase 2 | Pgm2 | 72157 | 1.54 | 0.00 | 13.57 |
| 1451385_PM_at | family with sequence similarity | Fam162a | 70186 | 1.67 | 0.00 | 15.91 |
| | 162, member A | | | | | |
| 1451386_PM_at | biliverdin reductase B (flavin | Blvrb | 233016 | 1.55 | 0.00 | 6.65 |
| | reductase (NADPH)) | | | | | |
| 1451765_PM_a_at | ectonucleoside triphosphate | Entpd5 | 12499 | 1.60 | 0.00 | 7.46 |
| | diphosphohydrolase 5 | | | | | |
| 1451814_PM_a_at | HIV-1 tat interactive protein 2, | Htatip2 | 53415 | 1.68 | 0.00 | 8.12 |
| | homolog (human) | | | | | |
| 1455454_PM_at | aldo-keto reductase family 1, | Akr1c19 | 432720 | 2.39 | 0.00 | 9.83 |
| | member C19 | | | | | |
| 1455595_PM_at | UDP glucuronosyltransferase 2 | Ugt2b36 | 231396 | 2.50 | 0.00 | 9.49 |
| | family, polypeptide B36 | | | | | |
| 1455959_PM_s_at | glutamate-cysteine ligase, catalytic | Gclc | 14629 | 2.48 | 0.00 | 12.53 |
| | subunit | | | | | |
| 1455978_PM_a_at | matrilin 2 | Matn2 | 17181 | 2.21 | 0.00 | 3.74 |
| 1460196_PM_at | carbonyl reductase 1 | Cbr1 | 12408 | 1.83 | 0.00 | 24.05 |
| 1460373_PM_a_at | SET domain containing 4 | Setd4 | 224440 | 1.77 | 0.00 | 11.06 |

100MMF-vs-Veh, 12 h

| | | | | | | |
|---|---|---|---|---|---|---|
| 1416368_PM_at | glutathione S-transferase, alpha 4 | Gsta4 | 14860 | 2.66 | 0.00 | 19.33 |
| 1416411_PM_at | glutathione S-transferase, mu 2 | Gstm2 | 14863 | 2.20 | 0.00 | 13.57 |
| 1416416_PM_x_at | glutathione S-transferase, mu 1 | Gstm1 | 14862 | 3.70 | 0.00 | 22.05 |
| 1417991_PM_at | deiodinase, iodothyronine, type I | Dio1 | 13370 | −1.51 | 0.00 | 1.69 |
| 1418601_PM_at | aldehyde dehydrogenase family 1, subfamily | Aldh1a7 | 26358 | 1.74 | 0.00 | 11.46 |
| | A7 | | | | | |
| 1418672_PM_at | aldo-keto reductase family 1, member C13 | Akr1c13 | 27384 | 2.01 | 0.00 | 16.07 |
| 1419510_PM_at | carboxylesterase 1E | Ces1e | 13897 | 2.77 | 0.00 | 20.69 |
| 1419622_PM_at | UDP glucuronosyltransferase 2 family, | Ugt2b5 | 22238 | 5.00 | 0.00 | 15.23 |
| | polypeptide B5 | | | | | |
| 1421040_PM_a_at | glutathione S-transferase, alpha 2 (Yc2) | Gsta2 | 14858 | 6.66 | 0.00 | 12.48 |
| 1421041_PM_s_at | glutathione S-transferase, alpha 2 (Yc2) | Gsta2 | 14858 | 2.09 | 0.00 | 20.09 |
| 1421816_PM_at | glutathione reductase | Gsr | 14782 | 2.67 | 0.00 | 7.27 |
| 1422000_PM_at | aldo-keto reductase family 1, member C12 | Akr1c12 | 622402 | 1.52 | 0.00 | 11.44 |
| 1422072_PM_a_at | glutathione S-transferase, mu 6 | Gstm6 | 14867 | 2.19 | 0.00 | 3.05 |
| 1422327_PM_s_at | glucose-6-phosphate dehydrogenase 2 | G6pd2 | 14380 | 1.83 | 0.00 | 6.55 |
| 1422438_PM_at | epoxide hydrolase 1, microsomal | Ephx1 | 13849 | 2.17 | 0.00 | 1.66 |
| 1422757_PM_at | solute carrier family 5 (neutral amino acid | Slc5a4b | 64454 | 2.16 | 0.00 | 26.29 |
| | transporters, system A), member 4b | | | | | |
| 1423436_PM_at | glutathione S-transferase, alpha 3 | Gsta3 | 14859 | 10.80 | 0.00 | 28.35 |
| 1423437_PM_at | glutathione S-transferase, alpha 3 | Gsta3 | 14859 | 7.97 | 0.00 | 21.49 |
| 1423627_PM_at | NAD(P)H dehydrogenase, quinone 1 | Nqo1 | 18104 | 2.34 | 0.00 | 18.44 |
| 1424266_PM_s_at | carboxylesterase 1F | Ces1f | 234564 | 6.54 | 0.00 | 27.69 |
| 1424835_PM_at | glutathione S-transferase, mu 4 | Gstm4 | 14865 | 5.43 | 0.00 | 17.82 |
| 1425239_PM_at | SET domain containing 4 | Setd4 | 224440 | 1.60 | 0.00 | 2.08 |
| 1425627_PM_x_at | glutathione S-transferase, mu 1 | Gstm1 | 14862 | 4.61 | 0.00 | 19.97 |
| 1427473_PM_at | glutathione S-transferase, mu 3 | Gstm3 | 14864 | 2.14 | 0.00 | 5.39 |
| 1427474_PM_s_at | glutathione S-transferase, mu 3 | Gstm3 | 14864 | 1.58 | 0.00 | 9.51 |
| 1427912_PM_at | carbonyl reductase 3 | Cbr3 | 109857 | 32.24 | 0.00 | 22.69 |
| 1428988_PM_at | ATP-binding cassette, sub-family C | Abcc3 | 76408 | 2.10 | 0.00 | 11.69 |
| | (CFTR/MRP), member 3 | | | | | |
| 1429001_PM_at | pirin | Pir | 69656 | 4.19 | 0.00 | 22.79 |
| 1431418_PM_at | RIKEN cDNA C030026M15 gene | C030026M15Rik | 77378 | −1.81 | 0.00 | 0.16 |
| 1437662_PM_at | acyl-CoA synthetase medium-chain family | Acsm5 | 272428 | 1.95 | 0.00 | 8.50 |
| | member 5 | | | | | |
| 1439624_PM_at | UDP glucuronosyltransferase 2 family, | Ugt2b35 | 243085 | 2.45 | 0.00 | 9.24 |
| | polypeptide B35 | | | | | |
| 1441931_PM_x_at | glutathione synthetase | Gss | 14854 | 1.55 | 0.00 | 0.88 |
| 1446542_PM_at | acyl-CoA synthetase short-chain family | Acss2 | 60525 | 1.52 | 0.00 | 4.58 |
| | member 2 | | | | | |
| 1448273_PM_at | glutathione synthetase | Gss | 14854 | 1.57 | 0.00 | 3.36 |
| 1448330_PM_at | glutathione S-transferase, mu 1 | Gstm1 | 14862 | 4.27 | 0.00 | 22.26 |
| 1448894_PM_at | aldo-keto reductase family 1, member B8 | Akr1b8 | 14187 | 18.78 | 0.00 | 31.18 |
| 1449279_PM_at | glutathione peroxidase 2 | Gpx2 | 14776 | 1.54 | 0.00 | 11.19 |
| 1449486_PM_at | carboxylesterase 1G | Ces1g | 12623 | 9.45 | 0.00 | 19.31 |
| 1450455_PM_s_at | aldo-keto reductase family 1, member C12 | Akr1c12 | 27384 | 1.76 | 0.00 | 19.14 |
| 1451386_PM_at | biliverdin reductase B (flavin reductase | Blvrb | 233016 | 1.54 | 0.00 | 6.30 |
| | (NADPH)) | | | | | |
| 1451642_PM_at | kinesin family member 1B | Kif1b | 16561 | −1.51 | 0.00 | 0.62 |
| 1455454_PM_at | aldo-keto reductase family 1, member C19 | Akr1c19 | 432720 | 3.53 | 0.00 | 22.88 |
| 1455595_PM_at | UDP glucuronosyltransferase 2 family, | Ugt2b36 | 231396 | 3.03 | 0.00 | 15.54 |
| | polypeptide B36 | | | | | |
| 1457554_PM_at | apolipoprotein B | Apob | 238055 | −1.54 | 0.00 | 1.46 |
| 1460373_PM_a_at | SET domain containing 4 | Setd4 | 224440 | 1.56 | 0.00 | 4.71 |

100DMF-vs-100MMF, 2 h

| | | | | | | |
|---|---|---|---|---|---|---|
| 1440882_PM_at | low density lipoprotein | Lrp8 | 16975 | −1.69 | 0.00 | 0.04 |
| | receptor-related protein 8, | | | | | |
| | apolipoprotein e receptor | | | | | |

100DMF-vs-100MMF, 7 h

| | | | | | | |
|---|---|---|---|---|---|---|
| 1417880_PM_at | glucose-6-phosphatase, | G6pc | 14377 | −2.45 | 0.00 | 0.25 |
| | catalytic | | | | | |
| 1425260_PM_at | albumin | Alb | 11657 | 9.97 | 0.00 | 0.40 |

100DMF-vs-100MMF, 12 h
None
KIDNEY
100 DMF-vs-Veh, 2 h

| | | | | | | |
|---|---|---|---|---|---|---|
| 1415834_PM_at | dual specificity phosphatase 6 | Dusp6 | 67603 | 1.52 | 0.00 | 3.06 |
| 1415940_PM_at | zinc finger, AN1-type domain 2A | Zfand2a | 100494 | 1.55 | 0.00 | 1.93 |
| 1415997_PM_at | thioredoxin interacting protein | Txnip | 56338 | −1.73 | 0.00 | 0.74 |
| 1416039_PM_x_at | cysteine rich protein 61 | Cyr61 | 16007 | 2.43 | 0.00 | 10.99 |
| 1416042_PM_s_at | nuclear autoantigenic sperm | Nasp | 50927 | 1.62 | 0.00 | 18.30 |
| | protein (histone-binding) | | | | | |
| 1416067_PM_at | interferon-related developmental | Ifrd1 | 15982 | 1.52 | 0.00 | 8.13 |
| | regulator 1 | | | | | |
| 1416077_PM_at | adrenomedullin | Adm | 11535 | 1.56 | 0.00 | 0.99 |
| 1416084_PM_at | zinc finger, AN1-type domain 5 | Zfand5 | 22682 | 1.63 | 0.00 | 8.55 |
| 1416085_PM_s_at | zinc finger, AN1-type domain 5 | Zfand5 | 22682 | 1.68 | 0.00 | 13.99 |
| 1416288_PM_at | DnaJ (Hsp40) homolog, | Dnaja1 | 15502 | 1.69 | 0.00 | 16.86 |
| | subfamily A, member 1 | | | | | |
| 1416442_PM_at | immediate early response 2 | Ier2 | 15936 | 1.75 | 0.00 | 19.47 |
| 1416600_PM_a_at | regulator of calcineurin 1 | Rcan1 | 54720 | 1.51 | 0.00 | 0.98 |
| 1416755_PM_at | DnaJ (Hsp40) homolog, | Dnajb1 | 81489 | 3.03 | 0.00 | 36.82 |
| | subfamily B, member 1 | | | | | |
| 1416756_PM_at | DnaJ (Hsp40) homolog, | Dnajb1 | 81489 | 4.36 | 0.00 | 35.56 |
| | subfamily B, member 1 | | | | | |
| 1417065_PM_at | early growth response 1 | Egr1 | 13653 | 4.60 | 0.00 | 13.08 |
| 1417406_PM_at | SERTA domain containing 1 | Sertad1 | 55942 | 2.06 | 0.00 | 29.20 |
| 1417479_PM_at | protein phosphatase 2, regulatory | Ppp2r3c | 59032 | 1.59 | 0.00 | 9.08 |
| | subunit B″, gamma | | | | | |
| 1417516_PM_at | DNA-damage inducible transcript 3 | Ddit3 | 13198 | 3.97 | 0.00 | 34.03 |
| 1417639_PM_at | solute carrier family 22 (organic | Slc22a4 | 30805 | −1.51 | 0.00 | 11.28 |
| | cation transporter), member 4 | | | | | |
| 1417930_PM_at | Ngfi-A binding protein 2 | Nab2 | 17937 | 2.74 | 0.00 | 26.23 |
| 1418025_PM_at | basic helix-loop-helix family, | Bhlhe40 | 20893 | 1.59 | 0.00 | 10.28 |
| | member e40 | | | | | |
| 1418203_PM_at | phorbol-12-myristate-13-acetate- | Pmaip1 | 58801 | 1.68 | 0.00 | 4.99 |
| | induced protein 1 | | | | | |
| 1418334_PM_at | DBF4 homolog (S. cerevisiae) | Dbf4 | 27214 | 2.08 | 0.00 | 5.98 |
| 1418349_PM_at | heparin-binding EGF-like growth | Hbegf | 15200 | 2.03 | 0.00 | 22.02 |
| | factor | | | | | |
| 1418350_PM_at | heparin-binding EGF-like growth | Hbegf | 15200 | 2.21 | 0.00 | 26.28 |
| | factor | | | | | |
| 1418370_PM_at | troponin C, cardiac/slow skeletal | Tnnc1 | 21924 | 1.89 | 0.00 | 7.95 |
| 1418401_PM_a_at | dual specificity phosphatase 16 | Dusp16 | 70686 | 1.50 | 0.00 | 26.35 |
| 1418571_PM_at | tumor necrosis factor receptor | Tnfrsf12a | 27279 | 2.37 | 0.00 | 9.98 |
| | superfamily, member 12a | | | | | |
| 1418572_PM_x_at | tumor necrosis factor receptor | Tnfrsf12a | 27279 | 2.33 | 0.00 | 9.32 |
| | superfamily, member 12a | | | | | |
| 1418591_PM_at | DnaJ (Hsp40) homolog, | Dnaja4 | 58233 | 2.05 | 0.00 | 8.65 |
| | subfamily A, member 4 | | | | | |
| 1418592_PM_at | DnaJ (Hsp40) homolog, | Dnaja4 | 58233 | 1.87 | 0.00 | 13.21 |
| | subfamily A, member 4 | | | | | |
| 1418627_PM_at | glutamate-cysteine ligase, | Gclm | 14630 | 1.51 | 0.00 | 7.53 |
| | modifier subunit | | | | | |
| 1418640_PM_at | sirtuin 1 (silent mating type | Sirt1 | 93759 | 1.68 | 0.00 | 21.63 |
| | information regulation 2, | | | | | |
| | homolog) 1 (S. cerevisiae) | | | | | |
| 1418918_PM_at | insulin-like growth factor binding | Igfbp1 | 16006 | −2.89 | 0.00 | 7.17 |
| | protein 1 | | | | | |
| 1418932_PM_at | nuclear factor, interleukin 3, | Nfil3 | 18030 | 2.29 | 0.00 | 2.17 |
| | regulated | | | | | |
| 1418936_PM_at | v-maf musculoaponeurotic | Maff | 17133 | 2.83 | 0.00 | 18.87 |
| | fibrosarcoma oncogene family, | | | | | |
| | protein F (avian) | | | | | |
| 1418949_PM_at | growth differentiation factor 15 | Gdf15 | 23886 | 5.39 | 0.00 | 37.31 |
| 1418955_PM_at | zinc finger protein 93 | Zfp93 | 22755 | −1.74 | 0.00 | 9.58 |
| 1418966_PM_a_at | discoidin, CUB and LCCL | Dcbld1 | 66686 | 1.78 | 0.00 | 21.00 |
| | domain containing 1 | | | | | |
| 1419051_PM_at | OVO homolog-like 1 | Ovol1 | 18426 | −1.60 | 0.00 | 1.46 |
| | (Drosophila) | | | | | |
| 1419086_PM_at | fibroblast growth factor binding | Fgfbp1 | 14181 | 1.81 | 0.00 | 4.06 |
| | protein 1 | | | | | |
| 1419140_PM_at | activin receptor IIB | Acvr2b | 11481 | −1.51 | 0.00 | 1.06 |
| 1419253_PM_at | methylenetetrahydrofolate | Mthfd2 | 17768 | 2.06 | 0.00 | 11.38 |
| | dehydrogenase (NAD+ | | | | | |
| | dependent), | | | | | |
| | methenyltetrahydrofolate | | | | | |
| | cyclohydrolase | | | | | |
| 1419254_PM_at | methylenetetrahydrofolate | Mthfd2 | 17768 | 1.54 | 0.00 | 6.93 |
| | dehydrogenase (NAD+ | | | | | |
| | dependent), | | | | | |
| | methenyltetrahydrofolate | | | | | |
| | cyclohydrolase | | | | | |
| 1419435_PM_at | aldehyde oxidase 1 | Aox1 | 11761 | 2.46 | 0.00 | 6.18 |
| 1419942_PM_at | Sulfiredoxin 1 homolog (S. cerevisiae) | Srxn1 | 76650 | 1.62 | 0.00 | 1.48 |
| 1420056_PM_s_at | jumonji domain containing 6 | Jmjd6 | 107817 | 1.52 | 0.00 | 17.28 |
| 1420057_PM_at | Jumonji domain containing 6 | Jmjd6 | 107817 | 1.54 | 0.00 | 26.46 |
| 1420342_PM_at | ganglioside-induced | Gdap10 | 14546 | 1.52 | 0.00 | 3.67 |
| | differentiation-associated-protein | | | | | |
| | 10 | | | | | |
| 1420990_PM_at | chromodomain helicase DNA | Chd1 | 12648 | 1.76 | 0.00 | 18.48 |
| | binding protein 1 | | | | | |
| 1421224_PM_a_at | HNF1 homeobox B | Hnf1b | 21410 | −1.77 | 0.00 | 2.81 |
| 1421262_PM_at | lipase, endothelial | Lipg | 16891 | 2.42 | 0.00 | 1.11 |
| 1421529_PM_a_at | thioredoxin reductase 1 | Txnrd1 | 50493 | 1.53 | 0.00 | 11.19 |
| 1422452_PM_at | BCL2-associated athanogene 3 | Bag3 | 29810 | 2.03 | 0.00 | 21.55 |
| 1422612_PM_at | hexokinase 2 | Hk2 | 15277 | 2.72 | 0.00 | 1.70 |
| 1423437_PM_at | glutathione S-transferase, alpha 3 | Gsta3 | 14859 | 2.01 | 0.00 | 5.57 |
| 1423481_PM_at | RIO kinase 2 (yeast) | Riok2 | 67045 | 1.62 | 0.00 | 11.46 |
| 1423502_PM_at | bromodomain containing 2 | Brd2 | 14312 | 1.83 | 0.00 | 21.57 |
| 1423549_PM_at | solute carrier family 1 | Slc1a4 | 55963 | 1.52 | 0.00 | 0.96 |
| | (glutamate/neutral amino acid | | | | | |
| | transporter), member 4 | | | | | |
| 1423566_PM_a_at | heat shock 105 kDa/110 kDa | Hsph1 | 15505 | 2.40 | 0.00 | 14.44 |
| | protein 1 | | | | | |
| 1423605_PM_a_at | transformed mouse 3T3 cell | Mdm2 | 17246 | 1.51 | 0.00 | 26.24 |
| | double minute 2 | | | | | |
| 1423706_PM_a_at | phosphogluconate dehydrogenase | Pgd | 110208 | 1.61 | 0.00 | 6.54 |
| 1423862_PM_at | pleckstrin homology domain | Plekhf2 | 71801 | 1.54 | 0.00 | 5.61 |
| | containing, family F (with FYVE | | | | | |
| | domain) member 2 | | | | | |
| 1424022_PM_at | oxidative stress induced growth | Osgin1 | 71839 | 4.63 | 0.00 | 38.98 |
| | inhibitor 1 | | | | | |
| 1424213_PM_at | UbiA prenyltransferase domain | Ubiad1 | 71707 | −1.61 | 0.00 | 8.44 |
| | containing 1 | | | | | |
| 1424296_PM_at | glutamate-cysteine ligase, | Gclc | 14629 | 1.70 | 0.00 | 1.17 |
| | catalytic subunit | | | | | |
| 1424380_PM_at | vacuolar protein sorting 37B | Vps37b | 330192 | 1.56 | 0.00 | 17.65 |
| | (yeast) | | | | | |
| 1424487_PM_x_at | thioredoxin reductase 1 | Txnrd1 | 50493 | 1.99 | 0.00 | 3.09 |
| 1424988_PM_at | myosin regulatory light chain | Mylip | 218203 | −1.70 | 0.00 | 3.70 |
| | interacting protein | | | | | |
| 1425185_PM_at | PPPDE peptidase domain | Pppde1 | 78825 | 1.94 | 0.00 | 35.48 |
| | containing 1 | | | | | |
| 1425287_PM_at | zinc finger protein 189 | Zfp189 | 230162 | 1.51 | 0.00 | 3.40 |
| 1425305_PM_at | zinc finger protein 295 | Zfp295 | 114565 | 2.68 | 0.00 | 32.59 |
| 1425656_PM_a_at | brain-specific angiogenesis | Baiap2 | 108100 | 1.51 | 0.00 | 4.79 |
| | inhibitor 1-associated protein 2 | | | | | |
| 1426381_PM_at | peroxisome proliferative activated | Pprc1 | 226169 | 1.74 | 0.00 | 13.21 |
| | receptor, gamma, coactivator- | | | | | |
| | related 1 | | | | | |
| 1426645_PM_at | heat shock protein 90, alpha | Hsp90aa1 | 15519 | 1.64 | 0.00 | 7.26 |
| | (cytosolic), class A member 1 | | | | | |
| 1426722_PM_at | solute carrier family 38, member | Slc38a2 | 67760 | 2.05 | 0.00 | 25.17 |
| 1426875_PM_s_at | sulfiredoxin 1 homolog (S. cerevisiae) | Srxn1 | 76650 | 1.59 | 0.00 | 1.56 |
| 1426936_PM_at | cDNA sequence BC005512 /// | BC005512 /// | 192885 | −1.67 | 0.00 | 2.46 |
| | RIKEN cDNA F630007L15 gene | F630007L15 | /// | | | |
| | /// predicted gene 6958 | Rik /// | 629242 | | | |
| | | Gm6958 | /// | | | |
| | | | 641366 | | | |
| 1427056_PM_at | a disintegrin-like and | Adamts15 | 235130 | −2.26 | 0.00 | 8.53 |
| | metallopeptidase (reprolysin type) | | | | | |
| | with thrombospondin type 1 | | | | | |
| | motif, 15 | | | | | |
| 1427126_PM_at | heat shock protein 1B | Hspa1b | 15511 | 3.65 | 0.00 | 11.66 |
| 1427127_PM_x_at | heat shock protein 1B | Hspa1b | 15511 | 3.37 | 0.00 | 15.10 |
| 1427369_PM_at | NLR family, pyrin domain | Nlrp6 | 101613 | −1.52 | 0.00 | 7.11 |
| | containing 6 | | | | | |
| 1427372_PM_at | cytochrome P450, family 27, | Cyp27b1 | 13115 | −3.22 | 0.00 | 0.95 |
| | subfamily b, polypeptide 1 | | | | | |
| 1427718_PM_a_at | transformed mouse 3T3 cell | Mdm2 | 17246 | 1.66 | 0.00 | 1.04 |
| | double minute 2 | | | | | |
| 1427912_PM_at | carbonyl reductase 3 | Cbr3 | 109857 | 3.04 | 0.00 | 2.02 |
| 1428223_PM_at | major facilitator superfamily | Mfsd2a | 76574 | −2.59 | 0.00 | 0.27 |
| | domain containing 2A | | | | | |
| 1428400_PM_at | RIKEN cDNA 2200002K05 gene | 2200002K05Rik | 69137 | −1.79 | 0.00 | 8.19 |
| 1428562_PM_at | RIKEN cDNA 2210403K04 gene | 2210403K04Rik | 100042498 | 1.86 | 0.00 | 5.53 |
| 1428654_PM_at | RIKEN cDNA 1200016B10 gene | 1200016B10Rik | 66875 | 1.59 | 0.00 | 17.84 |
| 1428686_PM_at | RIKEN cDNA 2810029C07 gene | 2810029C07Rik | 72670 | −1.58 | 0.00 | 9.51 |
| 1428694_PM_at | MIR17 host gene 1 (non-protein | Mir17hg | 75957 | 2.82 | 0.00 | 28.45 |
| | coding) | | | | | |
| 1428834_PM_at | dual specificity phosphatase 4 | Dusp4 | 319520 | 2.09 | 0.00 | 4.18 |
| 1428963_PM_at | RWD domain containing 2A | Rwdd2a | 69519 | 2.54 | 0.00 | 30.29 |
| 1428975_PM_at | sushi domain containing 3 | Susd3 | 66329 | −1.51 | 0.00 | 2.90 |
| 1428997_PM_at | PHD finger protein 23 | Phf23 | 78246 | 1.72 | 0.00 | 24.55 |
| 1429056_PM_at | N(alpha)-acetyltransferase 16, | Naa16 | 66897 | 1.71 | 0.00 | 16.78 |
| | NatA auxiliary subunit | | | | | |
| 1429350_PM_at | EP300 interacting inhibitor of | Eid3 | 66341 | 1.84 | 0.00 | 12.97 |
| | differentiation 3 | | | | | |
| 1429456_PM_a_at | polymerase (RNA) III (DNA | Polr3e | 26939 | −1.69 | 0.00 | 1.12 |
| | directed) polypeptide E | | | | | |
| 1429813_PM_at | pantothenate kinase 1 | Pank1 | 75735 | −1.59 | 0.00 | 3.53 |
| 1429863_PM_at | LON peptidase N-terminal | Lonrf3 | 74365 | 4.13 | 0.00 | 30.68 |
| | domain and ring finger 3 | | | | | |
| 1430244_PM_at | RIKEN cDNA 4921509J17 gene | 4921509J17Rik | 70857 | 1.60 | 0.00 | 4.99 |
| 1430392_PM_at | RIKEN cDNA 9530086O07 gene | 9530086O07Rik | 78741 | −1.52 | 0.00 | 1.27 |
| 1430593_PM_at | coiled-coil domain containing 30 | Ccdc30 | 73332 | −2.01 | 0.00 | 5.58 |
| 1430686_PM_at | RIKEN cDNA 4833418N02 gene | 4833418N02Rik | 74597 | −1.51 | 0.00 | 3.31 |
| 1430744_PM_at | napsin A aspartic peptidase | Napsa | 16541 | −4.53 | 0.00 | 36.84 |
| 1430783_PM_at | RIKEN cDNA 4932416J16 gene | 4932416J16Rik | 67541 | −1.67 | 0.00 | 8.92 |
| 1431018_PM_at | RIKEN cDNA 1810013L24 gene | 1810013L24Rik | 69053 | 1.50 | 0.00 | 1.83 |
| 1431140_PM_at | thioesterase superfamily member 4 | Them4 | 75778 | 1.80 | 0.00 | 7.45 |
| 1431182_PM_at | heat shock protein 8 /// | Hspa8 /// | 15481 /// | 2.63 | 0.00 | 6.77 |
| | hypothetical LOC624853 | LOC624853 | 624853 | | | |
| 1431254_PM_at | kelch repeat and BTB (POZ) | Kbtbd11 | 74901 | −1.77 | 0.00 | 10.21 |
| | domain containing 11 | | | | | |
| 1431734_PM_a_at | DnaJ (Hsp40) homolog, | Dnajb4 | 67035 | 2.15 | 0.00 | 19.23 |
| | subfamily B, member 4 | | | | | |
| 1431740_PM_at | solute carrier family 7, (cationic | Slc7a13 | 74087 | −2.60 | 0.00 | 3.50 |
| | amino acid transporter, y+ | | | | | |
| | system) member 13 | | | | | |
| 1431905_PM_s_at | RIKEN cDNA 4933427G17 gene | 4933427G17Rik | 74466 | −1.65 | 0.00 | 0.95 |
| 1432625_PM_at | RIKEN cDNA 5830487K18 gene | 5830487K18Rik | 76125 | −1.90 | 0.00 | 15.82 |
| 1432665_PM_at | RIKEN cDNA 2210416J07 gene | 2210416J07Rik | 72374 | −1.87 | 0.00 | 2.85 |
| 1432757_PM_at | RIKEN cDNA 2900011L18 gene | 2900011L18Rik | 77082 | 1.62 | 0.00 | 7.48 |
| 1433398_PM_at | FYVE, RhoGEF and PH domain | Fgd3 | 30938 | 1.66 | 0.00 | 8.14 |
| | containing 3 | | | | | |
| 1433599_PM_at | bromodomain adjacent to zinc | Baz1a | 217578 | 2.51 | 0.00 | 27.05 |
| | finger domain 1A | | | | | |
| 1433674_PM_a_at | small nucleolar RNA host gene | Snhg1 | 83673 | 1.62 | 0.00 | 31.12 |
| | (non-protein coding) 1 | | | | | |
| 1433675_PM_at | small nucleolar RNA host gene | Snhg1 | 83673 | 1.69 | 0.00 | 34.09 |
| | (non-protein coding) 1 | | | | | |
| 1433699_PM_at | tumor necrosis factor, alpha- | Tnfaip3 | 21929 | −1.83 | 0.00 | 4.35 |
| | induced protein 3 | | | | | |
| 1433944_PM_at | HECT domain containing 2 | Hectd2 | 226098 | 1.67 | 0.00 | 4.44 |
| 1433966_PM_x_at | asparagine synthetase | Asns | 27053 | 2.54 | 0.00 | 13.69 |
| 1434196_PM_at | DnaJ (Hsp40) homolog, | Dnaja4 | 58233 | 1.63 | 0.00 | 9.38 |
| | subfamily A, member 4 | | | | | |
| 1434496_PM_at | polo-like kinase 3 (Drosophila) | Plk3 | 12795 | 3.14 | 0.00 | 31.05 |
| 1434583_PM_at | transmembrane protein 26 | Tmem26 | 327766 | −1.66 | 0.00 | 5.11 |
| 1434660_PM_at | alkB, alkylation repair homolog 1 | Alkbh1 | 211064 | 1.53 | 0.00 | 14.97 |
| | (E. coli) | | | | | |
| 1434901_PM_at | zinc finger and BTB domain | Zbtb2 | 381990 | 2.52 | 0.00 | 37.72 |
| | containing 2 | | | | | |
| 1434967_PM_at | zinc finger, SWIM domain | Zswim6 | 67263 | 1.63 | 0.00 | 14.91 |
| | containing 6 | | | | | |
| 1435005_PM_at | centromere protein E | Cenpe | 229841 | −2.20 | 0.00 | 13.60 |
| 1435035_PM_at | RNA (guanine-9-) | Rg9mtd2 | 108943 | 1.82 | 0.00 | 11.44 |
| | methyltransferase domain | | | | | |
| | containing 2 | | | | | |
| 1435160_PM_at | AHA1, activator of heat shock | Ahsa2 | 268390 | 1.64 | 0.00 | 8.86 |
| | protein ATPase homolog 2 (yeast) | | | | | |
| 1435311_PM_s_at | synapsin III | Syn3 | 27204 | −1.73 | 0.00 | 2.56 |
| 1435409_PM_at | transmembrane protein 26 | Tmem26 | 327766 | −1.85 | 0.00 | 8.85 |
| 1435465_PM_at | kelch repeat and BTB (POZ) | Kbtbd11 | 74901 | −1.50 | 0.00 | 25.22 |
| | domain containing 11 | | | | | |
| 1435628_PM_x_at | cDNA sequence BC005512 /// | BC005512 /// | 192885 | −1.72 | 0.00 | 3.53 |
| | RIKEN cDNA F630007L15 gene | F630007L15Rik | /// | | | |
| | /// predicted gene 6958 | /// | 629242 | | | |
| | | Gm6958 | /// | | | |
| | | | 641366 | | | |
| 1435632_PM_at | nuclear fragile X mental | Nufip2 | 68564 | 1.57 | 0.00 | 4.20 |
| | retardation protein interacting | | | | | |
| | protein 2 | | | | | |
| 1435904_PM_at | eukaryotic translation initiation | Eif2c3 | 214150 | 1.53 | 0.00 | 6.78 |
| | factor 2C, 3 | | | | | |
| 1435912_PM_at | UBX domain protein 7 | Ubxn7 | 224111 | 1.51 | 0.00 | 18.53 |
| 1436200_PM_at | LON peptidase N-terminal | Lonrf3 | 74365 | 3.86 | 0.00 | 23.76 |
| | domain and ring finger 3 | | | | | |
| 1436357_PM_at | predicted gene 10374 | Gm10374 | 100191074 | −1.52 | 0.00 | 10.19 |
| 1436366_PM_at | protein phosphatase 1, regulatory | Ppp1r15b | 108954 | 1.72 | 0.00 | 8.29 |
| | (inhibitor) subunit 15b | | | | | |
| 1436521_PM_at | solute carrier family 36 | Slc36a2 | 246049 | 4.63 | 0.00 | 1.33 |
| | (proton/amino acid symporter), | | | | | |
| | member 2 | | | | | |
| 1436550_PM_at | F-box protein 30 | Fbxo30 | 71865 | 1.81 | 0.00 | 12.63 |
| 1436725_PM_at | RIKEN cDNA E130306D19 gene | E130306D19Rik | 230098 | −1.64 | 0.00 | 8.65 |
| 1436771_PM_x_at | phosphogluconate dehydrogenase | Pgd | 110208 | 1.52 | 0.00 | 10.54 |
| 1436871_PM_at | serine/arginine-rich splicing factor 7 | Srsf7 | 225027 | 1.57 | 0.00 | 12.68 |
| 1436974_PM_at | transmembrane protein 88B | Tmem88b | 320587 | −2.24 | 0.00 | 26.23 |
| 1437199_PM_at | dual specificity phosphatase 5 | Dusp5 | 240672 | 3.68 | 0.00 | 27.97 |
| 1437210_PM_a_at | bromodomain containing 2 | Brd2 | 14312 | 2.07 | 0.00 | 31.59 |
| 1437380_PM_x_at | phosphogluconate dehydrogenase | Pgd | 110208 | 1.53 | 0.00 | 9.92 |
| 1437410_PM_at | aldehyde dehydrogenase 2, | Aldh2 | 11669 | 1.69 | 0.00 | 13.15 |
| | mitochondrial | | | | | |
| 1437497_PM_a_at | heat shock protein 90, alpha | Hsp90aa1 | 15519 | 1.55 | 0.00 | 11.03 |
| | (cytosolic), class A member 1 | | | | | |
| 1437884_PM_at | ADP-ribosylation factor-like 5B | Arl5b | 75869 | 1.93 | 0.00 | 9.91 |
| 1438130_PM_at | TAF15 RNA polymerase II, | Taf15 | 70439 | 1.59 | 0.00 | 12.87 |
| | TATA box binding protein | | | | | |
| | (TBP)-associated factor | | | | | |
| 1438133_PM_a_at | cysteine rich protein 61 | Cyr61 | 16007 | 2.64 | 0.00 | 11.87 |
| 1438535_PM_at | pleckstrin homology domain | Phip | 83946 | 1.50 | 0.00 | 11.57 |
| | interacting protein | | | | | |
| 1438578_PM_a_at | BTB (POZ) domain containing 10 | Btbd10 | 68815 | 1.60 | 0.00 | 12.16 |
| 1438627_PM_x_at | phosphogluconate dehydrogenase | Pgd | 110208 | 1.50 | 0.00 | 8.47 |
| 1438664_PM_at | protein kinase, cAMP dependent | Prkar2b | 19088 | 2.43 | 0.00 | 0.58 |
| | regulatory, type II beta | | | | | |
| 1438725_PM_at | mediator complex subunit 13 | Med13 | 327987 | 2.02 | 0.00 | 13.91 |
| 1438764_PM_at | annexin A7 | Anxa7 | 11750 | 1.88 | 0.00 | 9.07 |
| 1438784_PM_at | B-cell leukemia/lymphoma 11B | Bcl11b | 58208 | −1.52 | 0.00 | 7.63 |
| 1438803_PM_s_at | sorting nexin 16 | Snx16 | 74718 | 1.60 | 0.00 | 7.06 |
| 1438842_PM_at | mitochondrial carrier homolog 2 | Mtch2 | 56428 | 1.67 | 0.00 | 5.56 |
| | (C. elegans) | | | | | |
| 1438879_PM_at | — | — | — | −1.61 | 0.00 | 1.24 |
| 1438880_PM_at | RIKEN cDNA 1700012D14 gene | 1700012D14Rik | 75479 | −1.55 | 0.00 | 6.59 |
| 1438992_PM_x_at | activating transcription factor 4 | Atf4 | 11911 | 1.77 | 0.00 | 21.35 |
| 1439093_PM_at | heat shock protein 4 like | Hspa4l | 18415 | 1.67 | 0.00 | 9.18 |
| 1439094_PM_at | clathrin, heavy polypeptide (Hc) | Cltc | 67300 | 2.07 | 0.00 | 20.23 |
| 1439292_PM_at | — | — | — | 2.14 | 0.00 | 14.73 |
| 1439293_PM_at | cDNA sequence BC031353 | BC031353 | 235493 | −1.84 | 0.00 | 4.72 |
| 1439352_PM_at | tripartite motif-containing 7 | Trim7 | 94089 | −1.54 | 0.00 | 6.55 |
| 1439669_PM_at | RIKEN cDNA 6430571L13 gene | 6430571L13Rik | 235599 | −1.85 | 0.00 | 0.41 |
| 1439680_PM_at | tumor necrosis factor (ligand) | Tnfsf10 | 22035 | −1.52 | 0.00 | 5.66 |
| | superfamily, member 10 | | | | | |
| 1440076_PM_at | — | — | — | 3.71 | 0.00 | 44.93 |
| 1440084_PM_at | — | — | — | −1.57 | 0.00 | 5.63 |
| 1440255_PM_at | histone H4 transcription factor | Hinfp | 102423 | 1.57 | 0.00 | 7.67 |
| 1440304_PM_at | — | — | — | −1.68 | 0.00 | 1.49 |
| 1440346_PM_at | KDM1 lysine (K)-specific | Kdm6b | 216850 | 1.52 | 0.00 | 23.26 |
| | demethylase 6B | | | | | |
| 1440457_PM_at | — | — | — | 1.76 | 0.00 | 7.05 |
| 1440660_PM_at | — | — | — | −1.56 | 0.00 | 5.28 |
| 1440671_PM_at | RIKEN cDNA A130012E19 gene | A130012E19Rik | 320235 | 1.56 | 0.00 | 4.01 |
| 1440765_PM_at | Fraser syndrome 1 homolog | Fras1 | 231470 | −1.73 | 0.00 | 12.40 |
| | (human) | | | | | |
| 1440831_PM_at | BTB and CNC homology 1 | Bach1 | 12013 | 1.90 | 0.00 | 36.70 |
| 1440867_PM_at | sprouty homolog 4 (Drosophila) | Spry4 | 24066 | 1.71 | 0.00 | 1.48 |
| 1441010_PM_at | hypothetical LOC100502834 | LOC100502834 | 100502834 | −1.53 | 0.00 | 0.37 |
| 1441042_PM_at | fibroblast growth factor 1 | Fgf1 | 14164 | −2.91 | 0.00 | 27.62 |
| 1441155_PM_at | — | — | — | 1.51 | 0.00 | 2.50 |
| 1441190_PM_at | actin related protein 2/3 complex, | Arpc5l | 74192 | 1.70 | 0.00 | 3.83 |
| | subunit 5-like | | | | | |
| 1441198_PM_at | zinc finger protein 39 | Zfp39 | 22698 | −1.64 | 0.00 | 13.58 |
| 1441360_PM_at | — | — | — | 1.55 | 0.00 | 16.43 |
| 1441413_PM_at | — | — | — | 1.88 | 0.00 | 13.72 |
| 1441455_PM_at | — | — | — | 1.58 | 0.00 | 7.96 |
| 1441482_PM_at | — | — | — | −2.08 | 0.00 | 4.14 |
| 1441484_PM_at | — | — | — | −1.79 | 0.00 | 6.31 |
| 1441518_PM_at | — | — | — | −1.67 | 0.00 | 9.30 |
| 1441546_PM_at | Membrane protein, palmitoylated | Mpp6 | 56524 | 1.62 | 0.00 | 1.38 |
| | 6 (MAGUK p55 subfamily | | | | | |
| | member 6) | | | | | |
| 1441604_PM_at | Esterase D/formylglutathione | Esd | 13885 | 1.62 | 0.00 | 15.74 |
| | hydrolase | | | | | |
| 1441759_PM_at | predicted gene 10804 | Gm10804 | 100038525 | −1.67 | 0.00 | 23.91 |
| 1441794_PM_at | — | — | — | −1.72 | 0.00 | 15.59 |
| 1441955_PM_s_at | polyadenylate binding protein- | Paip1 | 218693 | 1.61 | 0.00 | 1.91 |
| | interacting protein 1 | | | | | |
| 1441973_PM_at | zinc finger protein 295 | Zfp295 | 114565 | 2.08 | 0.00 | 13.48 |
| 1441988_PM_at | protein phosphatase 1K (PP2C | Ppm1k | 243382 | −1.62 | 0.00 | 4.85 |
| | domain containing) | | | | | |
| 1442071_PM_at | ATP-binding cassette, sub-family | Abce1 | 24015 | 1.53 | 0.00 | 9.47 |
| | E (OABP), member 1 | | | | | |
| 1442422_PM_at | — | — | — | 1.74 | 0.00 | 9.03 |
| 1442436_PM_at | fructosamine 3 kinase | Fn3k | 63828 | −1.60 | 0.00 | 2.17 |
| 1442483_PM_at | — | — | — | −1.59 | 0.00 | 0.33 |
| 1442504_PM_at | — | — | — | −1.70 | 0.00 | 2.31 |
| 1442506_PM_at | — | — | — | 5.76 | 0.00 | 35.71 |
| 1442546_PM_at | RIKEN cDNA C730027H18 gene | C730027H18Rik | 319572 | −1.57 | 0.00 | 5.32 |
| 1442671_PM_at | — | — | — | 1.55 | 0.00 | 16.49 |
| 1442725_PM_at | — | — | — | −1.53 | 0.00 | 6.95 |
| 1442726_PM_s_at | expressed sequence AI845619 | AI845619 | 103846 | 5.44 | 0.00 | 38.80 |
| 1442928_PM_at | — | — | — | 1.66 | 0.00 | 9.46 |
| 1443100_PM_at | — | — | — | −1.63 | 0.00 | 2.23 |
| 1443109_PM_at | — | — | — | 1.69 | 0.00 | 9.95 |
| 1443116_PM_at | proteasome (prosome, macropain) | Psme4 | 103554 | 1.56 | 0.00 | 6.20 |
| | activator subunit 4 | | | | | |
| 1443159_PM_at | RIKEN cDNA 9130221J17 gene | 9130221J17Rik | 319693 | 9.22 | 0.00 | 52.52 |
| 1443289_PM_at | — | — | — | −1.65 | 0.00 | 0.36 |
| 1443335_PM_at | — | — | — | −1.78 | 0.00 | 4.17 |
| 1443422_PM_at | RIKEN cDNA 2410089E03 gene | 2410089E03Rik | 73692 | −1.72 | 0.00 | 0.66 |
| 1443546_PM_at | — | — | — | 1.74 | 0.00 | 2.64 |
| 1443566_PM_at | — | — | — | −1.76 | 0.00 | 2.69 |
| 1443673_PM_x_at | — | — | — | 2.06 | 0.00 | 20.23 |
| 1443897_PM_at | DNA-damage inducible transcript 3 | Ddit3 | 13198 | 3.97 | 0.00 | 28.63 |
| 1443908_PM_at | hypothetical LOC100503447 | LOC100503447 | 100503447 | −1.72 | 0.00 | 11.01 |
| 1444021_PM_at | — | — | — | 3.24 | 0.00 | 34.46 |
| 1444057_PM_at | — | — | — | 1.75 | 0.00 | 21.77 |
| 1444111_PM_at | — | — | — | −1.67 | 0.00 | 17.25 |
| 1444212_PM_at | — | — | — | 1.57 | 0.00 | 9.69 |
| 1444227_PM_at | — | — | — | 1.56 | 0.00 | 4.53 |
| 1444265_PM_at | — | — | — | 1.93 | 0.00 | 9.74 |
| 1445032_PM_at | — | — | — | 1.96 | 0.00 | 4.34 |
| 1445089_PM_at | DNA segment, Chr 16, ERATO | D16Ertd778e | 52714 | −2.60 | 0.00 | 10.73 |
| | Doi 778, expressed | | | | | |
| 1445290_PM_at | — | — | — | −1.52 | 0.00 | 0.96 |
| 1445349_PM_at | — | — | — | −1.70 | 0.00 | 5.14 |
| 1445600_PM_at | — | — | — | −1.65 | 0.00 | 4.50 |
| 1445664_PM_at | — | — | — | −1.60 | 0.00 | 0.75 |
| 1445669_PM_at | sprouty homolog 4 (Drosophila) | Spry4 | 24066 | 1.54 | 0.00 | 2.73 |
| 1445857_PM_at | cDNA sequence BC026585 | BC026585 | 226527 | −1.51 | 0.00 | 5.92 |
| 1446075_PM_at | — | — | — | −1.62 | 0.00 | 15.20 |
| 1446172_PM_at | — | — | — | 1.85 | 0.00 | 12.58 |
| 1446303_PM_at | insulin-like growth factor I | Igf1r | 16001 | −1.66 | 0.00 | 1.79 |
| | receptor | | | | | |
| 1446621_PM_at | — | — | — | 1.74 | 0.00 | 6.59 |
| 1446921_PM_at | — | — | — | 1.58 | 0.00 | 8.36 |
| 1447172_PM_at | — | — | — | −1.61 | 0.00 | 11.40 |
| 1447396_PM_at | — | — | — | −1.90 | 0.00 | 8.52 |
| 1447411_PM_at | — | — | — | 4.68 | 0.00 | 26.35 |
| 1447818_PM_x_at | Ras homolog enriched in brain | Rhebl1 | 69159 | −1.57 | 0.00 | 5.99 |
| | like 1 | | | | | |
| 1447930_PM_at | bromodomain adjacent to zinc | Baz1a /// | 100505185 | 2.56 | 0.00 | 22.77 |
| | finger domain 1A /// | LOC100505185 | /// | | | |
| | bromodomain adjacent to zinc | | 217578 | | | |
| | finger domain protein 1A-like | | | | | |
| 1448135_PM_at | activating transcription factor 4 | Atf4 | 11911 | 2.12 | 0.00 | 44.45 |
| 1448170_PM_at | seven in absentia 2 | Siah2 | 20439 | 1.71 | 0.00 | 6.52 |
| 1448239_PM_at | heme oxygenase (decycling) 1 | Hmox1 | 15368 | 6.27 | 0.00 | 9.04 |
| 1448566_PM_at | solute carrier family 40 (iron- | Slc40a1 | 53945 | 1.71 | 0.00 | 8.18 |
| | regulated transporter), member 1 | | | | | |
| 1448568_PM_a_at | solute carrier family 20, member 1 | Slc20a1 | 20515 | 2.07 | 0.00 | 18.05 |
| 1448694_PM_at | Jun oncogene | Jun | 16476 | 1.50 | 0.00 | 8.53 |
| 1449311_PM_at | BTB and CNC homology 1 | Bach1 | 12013 | 2.55 | 0.00 | 19.51 |
| 1449519_PM_at | growth arrest and DNA-damage- | Gadd45a | 13197 | 1.75 | 0.00 | 7.46 |
| | inducible 45 alpha | | | | | |
| 1449813_PM_at | zinc finger protein 30 | Zfp30 | 22693 | −1.55 | 0.00 | 0.36 |
| 1449981_PM_a_at | N-acetyltransferase 2 (arylamine | Nat2 | 17961 | −2.01 | 0.00 | 3.70 |
| | N-acetyltransferase) | | | | | |
| 1450077_PM_at | chromodomain helicase DNA | Chd1 | 12648 | 1.67 | 0.00 | 23.61 |
| | binding protein 1 | | | | | |
| 1450188_PM_s_at | lipase, endothelial | Lipg | 16891 | 1.94 | 0.00 | 1.70 |
| 1450512_PM_at | netrin 4 | Ntn4 | 57764 | −1.67 | 0.00 | 2.68 |
| 1450716_PM_at | a disintegrin-like and | Adamts1 | 11504 | 1.86 | 0.00 | 13.62 |
| | metallopeptidase (reprolysin type) | | | | | |
| | with thrombospondin type 1 | | | | | |
| | motif, 1 | | | | | |
| 1450724_PM_at | family with sequence similarity | Fam126a | 84652 | 1.70 | 0.00 | 21.11 |
| | 126, member A | | | | | |
| 1450957_PM_a_at | sequestosome 1 | Sqstm1 | 18412 | 1.69 | 0.00 | 24.51 |
| 1451083_PM_s_at | alanyl-tRNA synthetase | Aars | 234734 | 1.53 | 0.00 | 12.52 |
| 1451095_PM_at | asparagine synthetase | Asns | 27053 | 3.03 | 0.00 | 17.46 |
| 1451177_PM_at | DnaJ (Hsp40) homolog, | Dnajb4 | 67035 | 2.48 | 0.00 | 30.50 |
| | subfamily B, member 4 | | | | | |
| 1451382_PM_at | ChaC, cation transport regulator- | Chac1 | 69065 | 31.00 | 0.00 | 45.85 |
| | like 1 (E. coli) | | | | | |
| 1451463_PM_at | proline rich 5 (renal) | Prr5 | 109270 | 1.65 | 0.00 | 6.09 |
| 1451516_PM_at | Ras homolog enriched in brain | Rhebl1 | 69159 | −2.01 | 0.00 | 6.26 |
| | like 1 | | | | | |
| 1451612_PM_at | metallothionein 1 | Mt1 | 17748 | 2.79 | 0.00 | 12.01 |
| 1451621_PM_at | PPPDE peptidase domain | Pppde1 | 78825 | 1.65 | 0.00 | 11.93 |
| | containing 1 | | | | | |
| 1451687_PM_a_at | HNF1 homeobox B | Hnf1b | 21410 | −1.82 | 0.00 | 5.02 |
| 1451692_PM_at | transmembrane and coiled-coil | Tmco6 | 71983 | −1.57 | 0.00 | 15.00 |
| | domains 6 | | | | | |
| 1452094_PM_at | procollagen-proline, 2- | P4ha1 | 18451 | 1.56 | 0.00 | 7.95 |
| | oxoglutarate 4-dioxygenase | | | | | |
| | (proline 4-hydroxylase), alpha 1 | | | | | |
| | polypeptide | | | | | |
| 1452218_PM_at | coiled-coil domain containing 117 | Ccdc117 | 104479 | 1.54 | 0.00 | 1.36 |
| 1452239_PM_at | gene trap ROSA 26, Philippe | Gt(ROSA)26Sor | 14910 | 1.62 | 0.00 | 17.77 |
| | Soriano | | | | | |
| 1452308_PM_a_at | ATPase, Na+/K+ transporting, | Atp1a2 | 98660 | 1.84 | 0.00 | 0.79 |
| | alpha 2 polypeptide | | | | | |
| 1452318_PM_a_at | heat shock protein 1B | Hspa1b | 15511 | 1.70 | 0.00 | 6.72 |
| 1452388_PM_at | heat shock protein 1A | Hspa1a | 193740 | 2.35 | 0.00 | 8.94 |
| 1452394_PM_at | cysteinyl-tRNA synthetase | Cars | 27267 | 1.63 | 0.00 | 2.91 |
| 1452623_PM_at | zinc finger protein 759 | Zfp759 | 268670 | −1.65 | 0.00 | 2.51 |
| 1452859_PM_at | RIKEN cDNA 1200016B10 gene | 1200016B10Rik | 66875 | 1.62 | 0.00 | 15.02 |
| 1452962_PM_at | transmembrane protein 25 | Tmem25 | 71687 | −1.52 | 0.00 | 11.88 |
| 1452975_PM_at | alanine-glyoxylate | Agxt2l1 | 71760 | −2.10 | 0.00 | 8.35 |
| | aminotransferase 2-like 1 | | | | | |
| 1453136_PM_at | F-box protein 30 | Fbxo30 | 71865 | 2.22 | 0.00 | 26.46 |
| 1453137_PM_at | F-box protein 30 | Fbxo30 | 71865 | 1.97 | 0.00 | 12.76 |
| 1453596_PM_at | inhibitor of DNA binding 2 | Id2 | 15902 | 1.83 | 0.00 | 5.75 |
| 1454109_PM_a_at | jumonji domain containing 6 | Jmjd6 | 107817 | 2.08 | 0.00 | 17.06 |
| 1454318_PM_at | RIKEN cDNA 2810403D21 gene | 2810403D21Rik | 69964 | −1.65 | 0.00 | 3.81 |
| 1454826_PM_at | zinc finger and BTB domain | Zbtb11 | 271377 | 1.60 | 0.00 | 19.90 |
| | containing 11 | | | | | |
| 1455087_PM_at | DNA segment, Chr 7, ERATO | D7Ertd715e | 52480 | −1.53 | 0.00 | 8.84 |
| | Doi 715, expressed | | | | | |
| 1455166_PM_at | ADP-ribosylation factor-like 5B | Arl5b | 75869 | 1.81 | 0.00 | 22.30 |
| 1455175_PM_at | PHD finger protein 13 | Phf13 | 230936 | 1.56 | 0.00 | 21.13 |
| 1455185_PM_s_at | PHD finger protein 16 | Phf16 | 382207 | 1.58 | 0.00 | 4.06 |
| 1455387_PM_at | nuclear fragile X mental | Nufip2 | 68564 | 1.70 | 0.00 | 19.41 |
| | retardation protein interacting | | | | | |
| | protein 2 | | | | | |
| 1455454_PM_at | aldo-keto reductase family 1, | Akr1c19 | 432720 | 1.53 | 0.00 | 1.79 |
| | member C19 | | | | | |
| 1455657_PM_at | SMG1 homolog, | Smg1 | 233789 | 1.69 | 0.00 | 1.87 |
| | phosphatidylinositol 3-kinase- | | | | | |
| | related kinase (C. elegans) | | | | | |
| 1455658_PM_at | CGG triplet repeat binding protein 1 | Cggbp1 | 106143 | 1.67 | 0.00 | 8.26 |
| 1455665_PM_at | LON peptidase N-terminal | Lonrf1 | 244421 | 1.96 | 0.00 | 17.63 |
| | domain and ring finger 1 | | | | | |
| 1455904_PM_at | growth arrest specific 5 /// small | Gas5 /// | 100217446 | 1.51 | 0.00 | 4.98 |
| | nucleolar RNA, C/D box 47 | Snord47 | /// | | | |
| | | | 14455 | | | |
| 1455959_PM_s_at | glutamate-cysteine ligase, | Gclc | 14629 | 1.56 | 0.00 | 2.30 |
| | catalytic subunit | | | | | |
| 1456041_PM_at | sorting nexin 16 | Snx16 | 74718 | 1.75 | 0.00 | 6.37 |
| 1456319_PM_at | — | — | — | −2.08 | 0.00 | 1.48 |
| 1456810_PM_at | vacuolar protein sorting 54 (yeast) | Vps54 | 245944 | 1.54 | 0.00 | 2.10 |
| 1456909_PM_at | glucose-6-phosphate isomerase- | LOC676974 | 676974 | 1.94 | 0.00 | 19.3 |
| | like | | | | | |
| 1456922_PM_at | sorting nexin 29 | Snx29 | 74478 | −1.64 | 0.00 | 10.75 |
| 1456955_PM_at | — | — | — | 1.59 | 0.00 | 21.92 |
| 1457110_PM_at | pantothenate kinase 1 | Pank1 | 75735 | −1.55 | 0.00 | 1.12 |
| 1457189_PM_at | — | — | — | −2.13 | 0.00 | 18.59 |
| 1457473_PM_at | chromodomain helicase DNA | Chd1 | 12648 | 1.60 | 0.00 | 10.06 |
| | binding protein 1 | | | | | |
| 1457552_PM_at | zinc finger protein 295 | Zfp295 | 114565 | 1.77 | 0.00 | 28.06 |
| 1457586_PM_at | — | — | — | 1.52 | 0.00 | 11.03 |
| 1458096_PM_at | — | — | — | −1.74 | 0.00 | 5.68 |
| 1458385_PM_at | heat shock protein 4 like | Hspa4l | 18415 | 1.62 | 0.00 | 1.59 |
| 1458452_PM_at | Ankyrin repeat domain 11 | Ankrd11 | 77087 | 1.51 | 0.00 | 4.46 |
| 1458503_PM_at | B-cell CLL/lymphoma 7A | Bcl7a | 77045 | −1.59 | 0.00 | 10.62 |
| 1458538_PM_at | — | — | — | 1.71 | 0.00 | 18.31 |
| 1459091_PM_at | — | — | — | 3.68 | 0.00 | 8.09 |
| 1459358_PM_at | — | — | — | 1.75 | 0.00 | 19.50 |
| 1459467_PM_at | expressed sequence AA986715 | AA986715 | 105449 | 1.72 | 0.00 | 18.62 |
| 1459516_PM_at | — | — | — | 1.88 | 0.00 | 12.14 |
| 1459585_PM_at | growth arrest specific 6 | Gas6 | 14456 | 1.59 | 0.00 | 14.29 |
| 1459722_PM_at | Zinc finger, SWIM domain | Zswim6 | 67263 | 1.52 | 0.00 | 5.00 |
| | containing 6 | | | | | |
| 1459774_PM_at | — | — | — | 1.68 | 0.00 | 9.37 |
| 1459913_PM_at | tumor necrosis factor (ligand) | Tnfsf10 | 22035 | −1.73 | 0.00 | 8.85 |
| | superfamily, member 10 | | | | | |
| 1460033_PM_at | RIKEN cDNA A330023F24 gene | A330023F24Rik | 320977 | 1.65 | 0.00 | 19.11 |
| 1460511_PM_at | plakophilin 2 | Pkp2 | 67451 | 1.66 | 0.00 | 18.71 |

100DMF-vs-Veh, 7 h

| | | | | | | |
|---|---|---|---|---|---|---|
| 1415983_PM_at | lymphocyte cytosolic protein 1 | Lcp1 | 18826 | 1.53 | 0.00 | 6.77 |
| 1416368_PM_at | glutathione S-transferase, alpha 4 | Gsta4 | 14860 | 1.51 | 0.00 | 1.45 |
| 1418358_PM_at | sperm mitochondria-associated | Smcp | 17235 | −1.84 | 0.00 | 1.76 |
| | cysteine-rich protein | | | | | |
| 1418571_PM_at | tumor necrosis factor receptor | Tnfrsf12a | 27279 | 1.94 | 0.00 | 4.85 |
| | superfamily, member 12a | | | | | |
| 1418572_PM_x_at | tumor necrosis factor receptor | Tnfrsf12a | 27279 | 1.83 | 0.00 | 2.94 |
| | superfamily, member 12a | | | | | |
| 1418949_PM_at | growth differentiation factor 15 | Gdf15 | 23886 | 1.75 | 0.00 | 1.66 |
| 1419253_PM_at | methylenetetrahydrofolate | Mthfd2 | 17768 | 1.65 | 0.00 | 3.74 |
| | dehydrogenase (NAD+ dependent), | | | | | |
| | methenyltetrahydrofolate | | | | | |
| | cyclohydrolase | | | | | |
| 1419435_PM_at | aldehyde oxidase 1 | Aox1 | 11761 | 1.95 | 0.00 | 1.42 |
| 1419942_PM_at | Sulfiredoxin 1 homolog (S. cerevisiae) | Srxn1 | 76650 | 1.99 | 0.00 | 10.26 |
| 1420696_PM_at | sema domain, immunoglobulin | Sema3c | 20348 | 1.62 | 0.00 | 2.32 |
| | domain (Ig), short basic domain, | | | | | |
| | secreted, (semaphorin) 3C | | | | | |
| 1421209_PM_s_at | inhibitor of kappaB kinase gamma | Ikbkg | 16151 | 1.64 | 0.00 | 9.65 |
| 1421529_PM_a_at | thioredoxin reductase 1 | Txnrd1 | 50493 | 1.54 | 0.00 | 13.57 |
| 1421609_PM_a_at | camello-like 3 | Cml3 | 93674 | −1.56 | 0.00 | 10.81 |
| 1422327_PM_s_at | glucose-6-phosphate dehydrogenase 2 | G6pd2 /// | 14380 | 1.61 | 0.00 | 7.38 |
| | /// glucose-6-phosphate | G6pdx | /// | | | |
| | dehydrogenase X-linked | | 14381 | | | |
| 1422557_PM_s_at | metallothionein 1 | Mt1 | 17748 | 1.66 | 0.00 | 8.19 |
| 1423186_PM_at | T-cell lymphoma invasion and | Tiam2 | 24001 | 1.84 | 0.00 | 8.11 |
| | metastasis 2 | | | | | |
| 1423436_PM_at | glutathione S-transferase, alpha 3 | Gsta3 | 14859 | 1.76 | 0.00 | 12.09 |
| 1423437_PM_at | glutathione S-transferase, alpha 3 | Gsta3 | 14859 | 2.14 | 0.00 | 8.91 |
| 1423627_PM_at | NAD(P)H dehydrogenase, quinone 1 | Nqo1 | 18104 | 1.71 | 0.00 | 15.80 |
| 1423706_PM_a_at | phosphogluconate dehydrogenase | Pgd | 110208 | 1.91 | 0.00 | 17.22 |
| 1424296_PM_at | glutamate-cysteine ligase, catalytic | Gclc | 14629 | 1.63 | 0.00 | 0.67 |
| | subunit | | | | | |
| 1424486_PM_a_at | thioredoxin reductase 1 | Txnrd1 | 50493 | 2.82 | 0.00 | 5.59 |
| 1424487_PM_x_at | thioredoxin reductase 1 | Txnrd1 | 50493 | 2.18 | 0.00 | 6.71 |
| 1424969_PM_s_at | uridine phosphorylase 2 | Upp2 | 76654 | −3.00 | 0.00 | 7.67 |
| 1425009_PM_at | t-complex-associated testis expressed 2 | Tcte2 | 21646 | −1.59 | 0.00 | 3.93 |
| 1425351_PM_at | sulfiredoxin 1 homolog (S. cerevisiae) | Srxn1 | 76650 | 2.01 | 0.00 | 4.98 |
| 1426300_PM_at | activated leukocyte cell adhesion | Alcam | 11658 | 2.08 | 0.00 | 14.28 |
| | molecule | | | | | |
| 1426301_PM_at | activated leukocyte cell adhesion | Alcam | 11658 | 1.88 | 0.00 | 13.26 |
| | molecule | | | | | |
| 1426399_PM_at | von Willebrand factor A domain | Vwal | 246228 | −1.51 | 0.00 | 3.46 |
| | containing 1 | | | | | |
| 1426875_PM_s_at | sulfiredoxin 1 homolog (S. cerevisiae) | Srxn1 | 76650 | 1.87 | 0.00 | 9.18 |
| 1426887_PM_at | nudix (nucleoside diphosphate linked | Nudt11 | 58242 | 1.55 | 0.00 | 8.63 |
| | moiety X)-type motif 11 | | | | | |
| 1427094_PM_at | polymerase (DNA directed), epsilon 2 | Pole2 | 18974 | 1.75 | 0.00 | 11.19 |
| | (p59 subunit) | | | | | |
| 1427320_PM_at | coatomer protein complex, subunit | Copg2as2 | 100044236 | 1.53 | 0.00 | 3.48 |
| | gamma 2, antisense 2 | | | | | |
| 1427912_PM_at | carbonyl reductase 3 | Cbr3 | 109857 | 5.88 | 0.00 | 14.82 |
| 1428223_PM_at | major facilitator superfamily domain | Mfsd2a | 76574 | −3.07 | 0.00 | 3.81 |
| | containing 2A | | | | | |
| 1428427_PM_at | F-box and leucine-rich repeat protein 2 | Fbxl2 | 72179 | −1.59 | 0.00 | 1.20 |
| 1428942_PM_at | metallothionein 2 | Mt2 | 17750 | 2.34 | 0.00 | 7.36 |
| 1429001_PM_at | pirin | Pir | 69656 | 2.35 | 0.00 | 19.49 |
| 1429895_PM_at | RIKEN cDNA 2310010G23 gene | 2310010G23Rik | 69591 | 2.05 | 0.00 | 0.96 |
| 1430111_PM_a_at | branched chain aminotransferase 1, | Bcat1 | 12035 | −1.56 | 0.00 | 0.50 |
| | cytosolic | | | | | |
| 1430135_PM_at | deoxyribonuclease II alpha | Dnase2a | 13423 | 1.69 | 0.00 | 6.38 |
| 1430744_PM_at | napsin A aspartic peptidase | Napsa | 16541 | −1.60 | 0.00 | 0.45 |
| 1433966_PM_x_at | asparagine synthetase | Asns | 27053 | 1.83 | 0.00 | 3.79 |
| 1434456_PM_at | RUN domain containing 3B | Rundc3b | 242819 | 1.55 | 0.00 | 2.61 |
| 1435311_PM_s_at | synapsin III | Syn3 | 27204 | −1.67 | 0.00 | 2.36 |
| 1435646_PM_at | inhibitor of kappaB kinase gamma | Ikbkg | 16151 | 1.77 | 0.00 | 12.07 |
| 1436101_PM_at | ring finger protein 24 | Rnf24 | 51902 | −1.88 | 0.00 | 7.68 |
| 1436210_PM_at | glycerol kinase 5 (putative) | Gk5 | 235533 | 1.51 | 0.00 | 2.65 |
| 1436771_PM_x_at | phosphogluconate dehydrogenase | Pgd | 110208 | 1.61 | 0.00 | 16.32 |
| 1436786_PM_at | RIKEN cDNA 1110069O07 gene | 1110069O07Rik | 71798 | −1.54 | 0.00 | 0.84 |
| 1436791_PM_at | wingless-related MMTV integration | Wnt5a | 22418 | 1.57 | 0.00 | 11.00 |
| | site 5A | | | | | |
| 1437380_PM_x_at | phosphogluconate dehydrogenase | Pgd | 110208 | 1.62 | 0.00 | 15.57 |
| 1437467_PM_at | activated leukocyte cell adhesion | Alcam | 11658 | 1.80 | 0.00 | 20.54 |
| | molecule | | | | | |
| 1440882_PM_at | low density lipoprotein receptor- | Lrp8 | 16975 | 2.13 | 0.00 | 1.75 |
| | related protein 8, apolipoprotein e | | | | | |
| | receptor | | | | | |
| 1440899_PM_at | flavin containing monooxygenase 5 | Fmo5 | 14263 | −1.51 | 0.00 | 6.43 |
| 1441042_PM_at | fibroblast growth factor 1 | Fgf1 | 14164 | −1.57 | 0.00 | 2.56 |
| 1441789_PM_at | — | — | — | −1.60 | 0.00 | 4.10 |
| 1441971_PM_at | — | — | — | 1.51 | 0.00 | 2.87 |
| 1442291_PM_at | lysophosphatidic acid receptor 2 | Lpar2 | 53978 | 1.66 | 0.00 | 2.20 |
| 1443870_PM_at | ATP-binding cassette, sub-family C | Abcc4 | 239273 | 1.78 | 0.00 | 14.16 |
| | (CFTR/MRP), member 4 | | | | | |
| 1444139_PM_at | DNA-damage-inducible transcript 4- | Ddit4l | 73284 | 1.56 | 0.00 | 4.78 |
| | like | | | | | |
| 1444682_PM_at | cDNA Sequence BC037032 | BC037032 | 414066 | 1.88 | 0.00 | 9.95 |
| 1447396_PM_at | — | — | — | −1.71 | 0.00 | 5.44 |
| 1448160_PM_at | lymphocyte cytosolic protein 1 | Lcp1 | 18826 | 1.61 | 0.00 | 4.34 |
| 1448239_PM_at | heme oxygenase (decycling) 1 | Hmox1 | 15368 | 5.61 | 0.00 | 8.73 |
| 1448354_PM_at | glucose-6-phosphate dehydrogenase | G6pdx | 14381 | 1.50 | 0.00 | 9.78 |
| | X-linked | | | | | |
| 1448568_PM_a_at | solute carrier family 20, member 1 | Slc20a1 | 20515 | 1.55 | 0.00 | 4.70 |
| 1448818_PM_at | wingless-related MMTV integration | Wnt5a | 22418 | 1.75 | 0.00 | 7.65 |
| | site 5A | | | | | |
| 1448894_PM_at | aldo-keto reductase family 1, member | Akr1b8 | 14187 | 1.62 | 0.00 | 1.20 |
| | B8 | | | | | |
| 1450869_PM_at | fibroblast growth factor 1 | Fgf1 | 14164 | −1.82 | 0.00 | 22.52 |
| 1450871_PM_a_at | branched chain aminotransferase 1, | Bcat1 | 12035 | −1.69 | 0.00 | 7.48 |
| | cytosolic | | | | | |
| 1451041_PM_at | Rho-associated coiled-coil containing | Rock2 | 19878 | 1.54 | 0.00 | 7.25 |
| | protein kinase 2 | | | | | |
| 1451095_PM_at | asparagine synthetase | Asns | 27053 | 1.92 | 0.00 | 4.01 |
| 1451386_PM_at | biliverdin reductase B (flavin | Blvrb | 233016 | 1.55 | 0.00 | 7.43 |
| | reductase (NADPH)) | | | | | |
| 1451548_PM_at | uridine phosphorylase 2 | Upp2 | 76654 | −3.28 | 0.00 | 6.94 |
| 1451680_PM_at | sulfiredoxin 1 homolog (S. cerevisiae) | Srxn1 | 76650 | 2.04 | 0.00 | 6.83 |
| 1451751_PM_at | DNA-damage-inducible transcript 4- | Ddit4l | 73284 | 2.11 | 0.00 | 8.10 |
| | like | | | | | |
| 1451828_PM_a_at | acyl-CoA synthetase long-chain | Acsl4 | 50790 | 1.53 | 0.00 | 2.68 |
| | family member 4 | | | | | |
| 1452733_PM_at | pantothenate kinase 2 | Pank2 | 74450 | −1.77 | 0.00 | 8.25 |
| 1453234_PM_at | RIKEN cDNA 1300002K09 gene | 1300002K09Rik | 74152 | 1.66 | 0.00 | 2.42 |
| 1453474_PM_at | abhydrolase domain containing 15 | Abhd15 | 67477 | 1.56 | 0.00 | 4.74 |
| 1454690_PM_at | inhibitor of kappaB kinase gamma | Ikbkg | 16151 | 1.54 | 0.00 | 20.75 |
| 1454992_PM_at | solute carrier family 7 (cationic | Slc7a1 | 11987 | 1.57 | 0.00 | 4.76 |
| | amino acid transporter, y+ system), | | | | | |
| | member 1 | | | | | |
| 1455454_PM_at | aldo-keto reductase family 1, member | Akr1c19 | 432720 | 1.64 | 0.00 | 5.73 |
| | C19 | | | | | |
| 1455699_PM_at | branched chain aminotransferase 1, | Bcat1 | 12035 | −2.11 | 0.00 | 2.56 |
| | cytosolic | | | | | |
| 1455959_PM_s_at | glutamate-cysteine ligase, catalytic | Gclc | 14629 | 1.61 | 0.00 | 4.29 |
| | subunit | | | | | |
| 1456509_PM_at | RIKEN cDNA 1110032F04 gene | 1110032F04Rik | 68725 | 4.29 | 0.00 | 12.31 |
| 1456524_PM_at | neuregulin 1 | Nrg1 | 211323 | 1.56 | 0.00 | 1.98 |
| 1456888_PM_at | 6-phosphofructo-2-kinase/fructose- | Pfkfb4 | 270198 | 1.55 | 0.00 | 2.48 |
| | 2,6-biphosphatase 4 | | | | | |
| 1457110_PM_at | pantothenate kinase 1 | Pank1 | 75735 | −1.64 | 0.00 | 4.06 |
| 1459091_PM_at | — | — | — | 2.25 | 0.00 | 0.03 |
| 1459625_PM_at | — | — | — | −1.65 | 0.00 | 1.44 |
| 1460632_PM_at | retinol dehydrogenase 10 (all-trans) | Rdh10 | 98711 | 1.59 | 0.00 | 4.56 |

100DMF-vs-Veh, 12 h

| | | | | | | |
|---|---|---|---|---|---|---|
| 1416101_PM_a_at | histone cluster 1, H1c | Hist1h1c | 50708 | 1.60 | 0.00 | 16.08 |
| 1416368_PM_at | glutathione S- | Gsta4 | 14860 | 1.89 | 0.00 | 11.39 |
| | transferase, alpha 4 | | | | | |
| 1418215_PM_at | meprin 1 beta | Mep1b | 17288 | −1.50 | 0.00 | 14.74 |
| 1418358_PM_at | sperm mitochondria- | Smcp | 17235 | −1.87 | 0.00 | 2.34 |
| | associated cysteine-rich | | | | | |
| | protein | | | | | |
| 1418601_PM_at | aldehyde | Aldh1a7 | 26358 | 2.48 | 0.00 | 9.09 |
| | dehydrogenase family | | | | | |
| | 1, subfamily A7 | | | | | |
| 1418627_PM_at | glutamate-cysteine | Gclm | 14630 | 1.50 | 0.00 | 8.72 |
| | ligase, modifier subunit | | | | | |
| 1418649_PM_at | EGL nine homolog 3 | Egln3 | 112407 | 1.63 | 0.00 | 12.07 |
| | (C. elegans) | | | | | |
| 1418847_PM_at | arginase type II | Arg2 | 11847 | 2.39 | 0.00 | 8.51 |
| 1418949_PM_at | growth differentiation | Gdf15 | 23886 | 1.77 | 0.00 | 1.93 |
| | factor 15 | | | | | |
| 1419253_PM_at | methylenetetrahydrofolate | Mthfd2 | 17768 | 1.60 | 0.00 | 2.59 |
| | dehydrogenase | | | | | |
| | (NAD+ dependent), | | | | | |
| | methenyltetrahydrofolate | | | | | |
| | cyclohydrolase | | | | | |
| 1419754_PM_at | myosin VA | Myo5a | 17918 | −2.68 | 0.00 | 21.59 |
| 1419942_PM_at | Sulfiredoxin 1 homolog | Srxn1 | 76650 | 1.89 | 0.00 | 8.22 |
| | (S. cerevisiae) | | | | | |
| 1421041_PM_s_at | predicted gene 3776 /// | Gm3776 /// | 100042295 | 1.53 | 0.00 | 8.66 |
| | glutathione S- | Gsta1 /// Gsta2 | /// | | | |
| | transferase, alpha 1 | | 14857 /// | | | |
| | (Ya) /// glutathione S- | | 14858 | | | |
| | transferase, alpha 2 | | | | | |
| | (Yc2) | | | | | |
| 1421108_PM_at | camello-like 2 | Cml2 | 93673 | 1.60 | 0.00 | 4.98 |
| 1421209_PM_s_at | inhibitor of kappaB | Ikbkg | 16151 | 1.50 | 0.00 | 4.85 |
| | kinase gamma | | | | | |
| 1421529_PM_a_at | thioredoxin reductase 1 | Txnrd1 | 50493 | 1.57 | 0.00 | 14.82 |
| 1421603_PM_a_at | carcinoembryonic | Ceacam2 | 26367 | −1.68 | 0.00 | 0.45 |
| | antigen-related cell | | | | | |
| | adhesion molecule 2 | | | | | |
| 1422327_PM_s_at | glucose-6-phosphate | G6pd2 /// | 14380 /// | 1.77 | 0.00 | 12.43 |
| | dehydrogenase 2 /// | G6pdx | 14381 | | | |
| | glucose-6-phosphate | | | | | |
| | dehydrogenase X-linked | | | | | |
| 1422533_PM_at | cytochrome P450, | Cyp51 | 13121 | −2.05 | 0.00 | 33.31 |
| | family 51 | | | | | |
| 1422534_PM_at | cytochrome P450, | Cyp51 | 13121 | −2.02 | 0.00 | 11.35 |
| | family 51 | | | | | |
| 1422606_PM_at | C1q and tumor necrosis | C1qtnf3 | 81799 | −1.67 | 0.00 | 6.27 |
| | factor related protein 3 | | | | | |
| 1423186_PM_at | T-cell lymphoma | Tiam2 | 24001 | 1.56 | 0.00 | 1.59 |
| | invasion and metastasis 2 | | | | | |
| 1423436_PM_at | glutathione S- | Gsta3 | 14859 | 1.97 | 0.00 | 18.57 |
| | transferase, alpha 3 | | | | | |
| 1423437_PM_at | glutathione S- | Gsta3 | 14859 | 2.66 | 0.00 | 16.97 |
| | transferase, alpha 3 | | | | | |
| 1423627_PM_at | NAD(P)H | Nqo1 | 18104 | 2.06 | 0.00 | 27.66 |
| | dehydrogenase, quinone 1 | | | | | |
| 1423706_PM_a_at | phosphogluconate | Pgd | 110208 | 2.20 | 0.00 | 25.08 |
| | dehydrogenase | | | | | |
| 1423954_PM_at | complement component 3 | C3 | 12266 | 1.90 | 0.00 | 3.69 |
| 1424126_PM_at | aminolevulinic acid | Alas1 | 11655 | 1.88 | 0.00 | 10.68 |
| | synthase 1 | | | | | |
| 1424296_PM_at | glutamate-cysteine | Gclc | 14629 | 1.60 | 0.00 | 0.15 |
| | ligase, catalytic subunit | | | | | |
| 1424486_PM_a_at | thioredoxin reductase 1 | Txnrd1 | 50493 | 2.31 | 0.00 | 1.51 |
| 1424487_PM_x_at | thioredoxin reductase 1 | Txnrd1 | 50493 | 1.91 | 0.00 | 2.88 |
| 1424626_PM_at | RIKEN cDNA | 2010003K11Rik | 69861 | 1.65 | 0.00 | 4.70 |
| | 2010003K11 gene | | | | | |
| 1424638_PM_at | cyclin-dependent kinase | Cdkn1a | 12575 | 2.02 | 0.00 | 0.72 |
| | inhibitor 1A (P21) | | | | | |
| 1424835_PM_at | glutathione S- | Gstm4 | 14865 | 1.52 | 0.00 | 13.55 |
| | transferase, mu 4 | | | | | |
| 1426047_PM_a_at | protein tyrosine | Ptprr | 19279 | 1.61 | 0.00 | 5.28 |
| | phosphatase, receptor | | | | | |
| | type, R | | | | | |
| 1426300_PM_at | activated leukocyte cell | Alcam | 11658 | 2.59 | 0.00 | 24.29 |
| | adhesion molecule | | | | | |
| 1426301_PM_at | activated leukocyte cell | Alcam | 11658 | 2.05 | 0.00 | 17.60 |
| | adhesion molecule | | | | | |
| 1426645_PM_at | heat shock protein 90, | Hsp90aa1 | 15519 | 1.57 | 0.00 | 6.12 |
| | alpha (cytosolic), class | | | | | |
| | A member 1 | | | | | |
| 1426875_PM_s_at | sulfiredoxin 1 homolog | Srxn1 | 76650 | 1.84 | 0.00 | 8.32 |
| | (S. cerevisiae) | | | | | |
| 1427912_PM_at | carbonyl reductase 3 | Cbr3 | 109857 | 5.67 | 0.00 | 14.10 |
| 1427932_PM_s_at | RIKEN cDNA | 1200003I10Rik | 319269 /// | 1.56 | 0.00 | 12.49 |
| | 1200003I10 gene /// | /// | 319443 /// | | | |
| | RIKEN cDNA | 1200015M12Rik | 71719 /// | | | |
| | 1200015M12 gene /// | /// | 71739 | | | |
| | RIKEN cDNA | A130040M12Rik | | | | |
| | A130040M12 gene /// | /// | | | | |
| | RIKEN cDNA | E430024C06Rik | | | | |
| | E430024C06 gene | | | | | |
| 1428023_PM_at | RIKEN cDNA | 3110009E18Rik | 73103 | 1.82 | 0.00 | 9.43 |
| | 3110009E18 gene | | | | | |
| 1428988_PM_at | ATP-binding cassette, | Abcc3 | 76408 | 1.67 | 0.00 | 1.32 |
| | sub-family C | | | | | |
| | (CFTR/MRP), member 3 | | | | | |
| 1429001_PM_at | pirin | Pir | 69656 | 2.89 | 0.00 | 28.82 |
| 1430111_PM_a_at | branched chain | Bcat1 | 12035 | −1.72 | 0.00 | 3.81 |
| | aminotransferase 1, | | | | | |
| | cytosolic | | | | | |
| 1430135_PM_at | deoxyribonuclease II | Dnase2a | 13423 | 2.12 | 0.00 | 16.76 |
| | alpha | | | | | |
| 1431320_PM_a_at | myosin VA | Myo5a | 17918 | −2.05 | 0.00 | 10.36 |
| 1433966_PM_x_at | asparagine synthetase | Asns | 27053 | 2.22 | 0.00 | 10.52 |
| 1434050_PM_at | vacuolar protein sorting | Vps8 | 209018 | −1.56 | 0.00 | 0.03 |
| | 8 homolog (S. cerevisiae) | | | | | |
| 1434716_PM_at | hepatitis A virus | Havcr1 | 171283 | 4.46 | 0.00 | 11.18 |
| | cellular receptor 1 | | | | | |
| 1434919_PM_at | alkylglycerone | Agps | 228061 | −1.55 | 0.00 | 8.44 |
| | phosphate synthase | | | | | |
| 1435646_PM_at | inhibitor of kappaB | Ikbkg | 16151 | 1.56 | 0.00 | 5.49 |
| | kinase gamma | | | | | |
| 1435663_PM_at | estrogen receptor 1 | Esr1 | 13982 | −1.54 | 0.00 | 5.12 |
| | (alpha) | | | | | |
| 1436051_PM_at | myosin VA | Myo5a | 17918 | −1.77 | 0.00 | 11.14 |
| 1436101_PM_at | ring finger protein 24 | Rnf24 | 51902 | −1.68 | 0.00 | 3.25 |
| 1436771_PM_x_at | phosphogluconate | Pgd | 110208 | 1.93 | 0.00 | 29.62 |
| | dehydrogenase | | | | | |
| 1437380_PM_x_at | phosphogluconate | Pgd | 110208 | 1.94 | 0.00 | 28.70 |
| | dehydrogenase | | | | | |
| 1437467_PM_at | activated leukocyte cell | Alcam | 11658 | 1.88 | 0.00 | 23.69 |
| | adhesion molecule | | | | | |
| 1437870_PM_at | solute carrier organic | Slco4c1 | 227394 | −1.90 | 0.00 | 12.94 |
| | anion transporter | | | | | |
| | family, member 4C1 | | | | | |
| 1438204_PM_at | Histone cluster 1, H1c | Hist1h1c | 50708 | 2.01 | 0.00 | 17.23 |
| 1438627_PM_x_at | phosphogluconate | Pgd | 110208 | 1.77 | 0.00 | 21.53 |
| | dehydrogenase | | | | | |
| 1438841_PM_s_at | arginase type II | Arg2 | 11847 | 2.24 | 0.00 | 7.52 |
| 1440008_PM_at | RIKEN cDNA | 2310043L19Rik | 75589 | 1.77 | 0.00 | 6.98 |
| | 2310043L19 gene | | | | | |
| 1440058_PM_at | solute carrier family 22 | Slc22a14 | 382113 | 1.59 | 0.00 | 9.02 |
| | (organic cation | | | | | |
| | transporter), member 14 | | | | | |
| 1440330_PM_at | Histone cluster 1, H2be | Hist1h2be | 319179 | 3.25 | 0.00 | 13.00 |
| 1441979_PM_at | cDNA sequence | BC060267 | 212516 | 1.80 | 0.00 | 12.78 |
| | BC060267 | | | | | |
| 1442145_PM_at | ATPase type 13A3 | Atp13a3 | 224088 | 1.64 | 0.00 | 19.14 |
| 1442291_PM_at | lysophosphatidic acid | Lpar2 | 53978 | 1.61 | 0.00 | 1.16 |
| | receptor 2 | | | | | |
| 1442588_PM_at | predicted gene 5101 | Gm5101 | 329217 | −1.52 | 0.00 | 13.43 |
| 1443870_PM_at | ATP-binding cassette, | Abcc4 | 239273 | 1.81 | 0.00 | 15.10 |
| | sub-family C | | | | | |
| | (CFTR/MRP), member 4 | | | | | |
| 1444487_PM_at | lecithin-retinol | Lrat | 79235 | 1.67 | 0.00 | 5.20 |
| | acyltransferase | | | | | |
| | (phosphatidylcholine- | | | | | |
| | retinol-O- | | | | | |
| | acyltransferase) | | | | | |
| 1445565_PM_at | Histone cluster 1, H2be | Hist1h2be | 319179 | 2.65 | 0.00 | 10.08 |
| 1446368_PM_at | RIKEN cDNA | 9130221J18Rik | 102123 | −1.51 | 0.00 | 6.77 |
| | 9130221J18 gene | | | | | |
| 1447356_PM_at | cDNA sequence | AB099516 /// | 380975 /// | −1.61 | 0.00 | 16.58 |
| | AB099516 /// HIG1 | Higd1c | 554292 | | | |
| | domain family, member | | | | | |
| | 1C | | | | | |
| 1448330_PM_at | glutathione S- | Gstm1 | 14862 | 1.54 | 0.00 | 34.69 |
| | transferase, mu 1 | | | | | |
| 1448354_PM_at | glucose-6-phosphate | G6pdx | 14381 | 1.55 | 0.00 | 11.95 |
| | dehydrogenase X-linked | | | | | |
| 1448766_PM_at | gap junction protein, | Gjb1 | 14618 | 1.59 | 0.00 | 8.47 |
| | beta 1 | | | | | |
| 1448894_PM_at | aldo-keto reductase | Akr1b8 | 14187 | 1.81 | 0.00 | 5.14 |
| | family 1, member B8 | | | | | |
| 1449002_PM_at | pleckstrin homology- | Phlda3 | 27280 | 1.57 | 0.00 | 1.10 |
| | like domain, family A, | | | | | |
| | member 3 | | | | | |
| 1449937_PM_at | endonuclease, polyU- | Endou | 19011 | 1.75 | 0.00 | 7.13 |
| | specific | | | | | |
| 1450702_PM_at | hemochromatosis | Hfe | 15216 | 1.60 | 0.00 | 15.86 |
| 1450871_PM_a_at | branched chain | Bcat1 | 12035 | −1.74 | 0.00 | 8.94 |
| | aminotransferase 1, | | | | | |
| | cytosolic | | | | | |
| 1451095_PM_at | asparagine synthetase | Asns | 27053 | 2.65 | 0.00 | 14.71 |
| 1451386_PM_at | biliverdin reductase B | Blvrb | 233016 | 1.94 | 0.00 | 20.75 |
| | (flavin reductase | | | | | |
| | (NADPH)) | | | | | |
| 1451680_PM_at | sulfiredoxin 1 homolog | Srxn1 | 76650 | 1.92 | 0.00 | 4.83 |
| | (S. cerevisiae) | | | | | |
| 1452733_PM_at | pantothenate kinase 2 | Pank2 | 74450 | −1.65 | 0.00 | 5.23 |
| 1452934_PM_at | transmembrane | Tmc5 | 74424 | 1.88 | 0.00 | 2.45 |
| | channel-like gene | | | | | |
| | family 5 | | | | | |
| 1452975_PM_at | alanine-glyoxylate | Agxt211 | 71760 | −1.65 | 0.00 | 1.26 |
| | aminotransferase 2-like 1 | | | | | |
| 1453234_PM_at | RIKEN cDNA | 1300002K09Rik | 74152 | 1.90 | 0.00 | 7.52 |
| | 1300002K09 gene | | | | | |
| 1454865_PM_at | solute carrier family 9 | Slc9a8 | 77031 | −1.98 | 0.00 | 17.89 |
| | (sodium/hydrogen | | | | | |
| | exchanger), member 8 | | | | | |
| 1455282_PM_x_at | aminolevulinic acid | Alas1 | 11655 | 1.62 | 0.00 | 6.88 |
| | synthase 1 | | | | | |
| 1455454_PM_at | aldo-keto reductase | Akr1c19 | 432720 | 1.92 | 0.00 | 12.92 |
| | family 1, member C19 | | | | | |
| 1455699_PM_at | branched chain | Bcat1 | 12035 | −1.92 | 0.00 | 0.40 |
| | aminotransferase 1, | | | | | |
| | cytosolic | | | | | |
| 1455959_PM_s_at | glutamate-cysteine | Gclc | 14629 | 1.56 | 0.00 | 2.99 |
| | ligase, catalytic subunit | | | | | |
| 1456722_PM_at | chordin-like 1 | Chrdl1 | 83453 | −1.71 | 0.00 | 5.91 |
| 1457110_PM_at | pantothenate kinase 1 | Pank1 | 75735 | −1.53 | 0.00 | 1.38 |
| 1458599_PM_at | — | — | — | 1.53 | 0.00 | 7.21 |
| 1459661_PM_at | doublecortin domain | Dcdc2a | 195208 | 1.76 | 0.00 | 6.89 |
| | containing 2a | | | | | |
| 1460196_PM_at | carbonyl reductase 1 | Cbr1 | 12408 | 1.80 | 0.00 | 18.85 |
| 1460616_PM_at | solute carrier organic | Slco4c1 | 227394 | −1.91 | 0.00 | 23.08 |
| | anion transporter | | | | | |
| | family, member 4C1 | | | | | |

100MMF-vs-Veh, 2 h

| | | | | | | |
|---|---|---|---|---|---|---|
| 1415834_PM_at | dual specificity phosphatase 6 | Dusp6 | 67603 | 1.57 | 0.00 | 5.13 |
| 1415940_PM_at | zinc finger, AN1-type domain 2A | Zfand2a | 100494 | 1.51 | 0.00 | 1.28 |
| 1415997_PM_at | thioredoxin interacting protein | Txnip | 56338 | −1.71 | 0.00 | 0.89 |
| 1416039_PM_x_at | cysteine rich protein 61 | Cyr61 | 16007 | 2.07 | 0.00 | 6.36 |
| 1416042_PM_s_at | nuclear autoantigenic sperm protein | Nasp | 50927 | 1.52 | 0.00 | 14.51 |
| | (histone-binding) | | | | | |
| 1416067_PM_at | interferon-related developmental | Ifrd1 | 15982 | 1.53 | 0.00 | 9.10 |
| | regulator 1 | | | | | |
| 1416077_PM_at | adrenomedullin | Adm | 11535 | 1.59 | 0.00 | 2.18 |
| 1416084_PM_at | zinc finger, AN1-type domain 5 | Zfand5 | 22682 | 1.70 | 0.00 | 11.59 |
| 1416085_PM_s_at | zinc finger, AN1-type domain 5 | Zfand5 | 22682 | 1.69 | 0.00 | 15.13 |
| 1416288_PM_at | DnaJ (Hsp40) homolog, subfamily | Dnaja1 | 15502 | 1.59 | 0.00 | 13.55 |
| | A, member 1 | | | | | |
| 1416442_PM_at | immediate early response 2 | Ier2 | 15936 | 1.86 | 0.00 | 24.53 |
| 1416497_PM_at | protein disulfide isomerase | Pdia4 | 12304 | −1.66 | 0.00 | 2.08 |
| | associated 4 | | | | | |
| 1416600_PM_a_at | regulator of calcineurin 1 | Rcan1 | 54720 | 1.51 | 0.00 | 1.36 |
| 1416755_PM_at | DnaJ (Hsp40) homolog, subfamily | Dnajb1 | 81489 | 2.63 | 0.00 | 31.16 |
| | B, member 1 | | | | | |
| 1416756_PM_at | DnaJ (Hsp40) homolog, subfamily | Dnajb1 | 81489 | 3.73 | 0.00 | 31.16 |
| | B, member 1 | | | | | |
| 1417065_PM_at | early growth response 1 | Egr1 | 13653 | 5.51 | 0.00 | 17.79 |
| 1417406_PM_at | SERTA domain containing 1 | Sertad1 | 55942 | 1.92 | 0.00 | 25.70 |
| 1417516_PM_at | DNA-damage inducible transcript 3 | Ddit3 | 13198 | 4.07 | 0.00 | 36.31 |
| 1417639_PM_at | solute carrier family 22 (organic | Slc22a4 | 30805 | −1.52 | 0.00 | 12.63 |
| | cation transporter), member 4 | | | | | |
| 1417930_PM_at | Ngfi-A binding protein 2 | Nab2 | 17937 | 2.33 | 0.00 | 19.87 |
| 1418334_PM_at | DBF4 homolog (S. cerevisiae) | Dbf4 | 27214 | 2.04 | 0.00 | 5.92 |
| 1418349_PM_at | heparin-binding EGF-like growth | Hbegf | 15200 | 2.27 | 0.00 | 29.72 |
| | factor | | | | | |
| 1418350_PM_at | heparin-binding EGF-like growth | Hbegf | 15200 | 2.63 | 0.00 | 37.21 |
| | factor | | | | | |
| 1418370_PM_at | troponin C, cardiac/slow skeletal | Tnnc1 | 21924 | 1.94 | 0.00 | 9.70 |
| 1418401_PM_a_at | dual specificity phosphatase 16 | Dusp16 | 70686 | 1.50 | 0.00 | 27.57 |
| 1418500_PM_at | nucleosome assembly protein 1-like 3 | Nap1l3 | 54561 | −1.53 | 0.00 | 1.18 |
| 1418571_PM_at | tumor necrosis factor receptor | Tnfrsf12a | 27279 | 2.32 | 0.00 | 10.05 |
| | superfamily, member 12a | | | | | |
| 1418572_PM_x_at | tumor necrosis factor receptor | Tnfrsf12a | 27279 | 2.24 | 0.00 | 8.74 |
| | superfamily, member 12a | | | | | |
| 1418591_PM_at | DnaJ (Hsp40) homolog, subfamily | Dnaja4 | 58233 | 1.87 | 0.00 | 5.93 |
| | A, member 4 | | | | | |
| 1418592_PM_at | DnaJ (Hsp40) homolog, subfamily | Dnaja4 | 58233 | 1.77 | 0.00 | 11.09 |
| | A, member 4 | | | | | |
| 1418640_PM_at | sirtuin 1 (silent mating type | Sirt1 | 93759 | 1.53 | 0.00 | 15.01 |
| | information regulation 2, homolog) | | | | | |
| | 1 (S. cerevisiae) | | | | | |
| 1418918_PM_at | insulin-like growth factor binding | Igfbp1 | 16006 | −2.27 | 0.00 | 2.38 |
| | protein 1 | | | | | |
| 1418932_PM_at | nuclear factor, interleukin 3, | Nfil3 | 18030 | 2.29 | 0.00 | 2.55 |
| | regulated | | | | | |
| 1418936_PM_at | v-maf musculoaponeurotic | Maff | 17133 | 3.80 | 0.00 | 30.85 |
| | fibrosarcoma oncogene family, | | | | | |
| | protein F (avian) | | | | | |
| 1418949_PM_at | growth differentiation factor 15 | Gdf15 | 23886 | 5.50 | 0.00 | 39.32 |
| 1418955_PM_at | zinc finger protein 93 | Zfp93 | 22755 | −1.62 | 0.00 | 6.69 |
| 1418966_PM_a_at | discoidin, CUB and LCCL domain | Dcbld1 | 66686 | 1.76 | 0.00 | 21.34 |
| | containing 1 | | | | | |
| 1419086_PM_at | fibroblast growth factor binding | Fgfbp1 | 14181 | 1.94 | 0.00 | 7.10 |
| | protein 1 | | | | | |
| 1419253_PM_at | methylenetetrahydrofolate | Mthfd2 | 17768 | 2.03 | 0.00 | 11.42 |
| | dehydrogenase (NAD+ dependent), | | | | | |
| | methenyltetrahydrofolate | | | | | |
| | cyclohydrolase | | | | | |
| 1419254_PM_at | methylenetetrahydrofolate | Mthfd2 | 17768 | 1.67 | 0.00 | 12.39 |
| | dehydrogenase (NAD+ dependent), | | | | | |
| | methenyltetrahydrofolate | | | | | |
| | cyclohydrolase | | | | | |
| 1419435_PM_at | aldehyde oxidase 1 | Aox1 | 11761 | 2.31 | 0.00 | 5.11 |
| 1419942_PM_at | Sulfiredoxin 1 homolog (S. cerevisiae) | Srxn1 | 76650 | 1.57 | 0.00 | 0.87 |
| 1420342_PM_at | ganglioside-induced differentiation- | Gdap10 | 14546 | 1.63 | 0.00 | 7.59 |
| | associated-protein 10 | | | | | |
| 1420990_PM_at | chromodomain helicase DNA | Chd1 | 12648 | 1.53 | 0.00 | 9.85 |
| | binding protein 1 | | | | | |
| 1421224_PM_a_at | HNF1 homeobox B | Hnf1b | 21410 | −1.59 | 0.00 | 0.02 |
| 1421262_PM_at | lipase, endothelial | Lipg | 16891 | 2.25 | 0.00 | 0.17 |
| 1422138_PM_at | plasminogen activator, urokinase | Plau | 18792 | −1.58 | 0.00 | 8.60 |
| 1422452_PM_at | BCL2-associated athanogene 3 | Bag3 | 29810 | 1.86 | 0.00 | 17.55 |
| 1423437_PM_at | glutathione S-transferase, alpha 3 | Gsta3 | 14859 | 1.93 | 0.00 | 4.85 |
| 1423481_PM_at | RIO kinase 2 (yeast) | Riok2 | 67045 | 1.58 | 0.00 | 10.44 |
| 1423502_PM_at | bromodomain containing 2 | Brd2 | 14312 | 1.50 | 0.00 | 9.16 |
| 1423566_PM_a_at | heat shock 105 kDa/110 kDa protein 1 | Hsph1 | 15505 | 2.22 | 0.00 | 12.19 |
| 1423672_PM_at | tetratricopeptide repeat domain 30B | Ttc30b | 72421 | −1.51 | 0.00 | 2.25 |
| 1423706_PM_a_at | phosphogluconate dehydrogenase | Pgd | 110208 | 1.51 | 0.00 | 4.01 |
| 1423862_PM_at | pleckstrin homology domain | Plekhf2 | 71801 | 1.50 | 0.00 | 4.98 |
| | containing, family F (with FYVE | | | | | |
| | domain) member 2 | | | | | |
| 1424022_PM_at | oxidative stress induced growth | Osgin1 | 71839 | 3.97 | 0.00 | 34.73 |
| | inhibitor 1 | | | | | |
| 1424296_PM_at | glutamate-cysteine ligase, catalytic | Gclc | 14629 | 1.66 | 0.00 | 0.79 |
| | subunit | | | | | |
| 1424508_PM_at | tetratricopeptide repeat domain 5 | Ttc5 | 219022 | −1.55 | 0.00 | 3.97 |
| 1424988_PM_at | myosin regulatory light chain | Mylip | 218203 | −1.67 | 0.00 | 3.53 |
| | interacting protein | | | | | |
| 1425185_PM_at | PPPDE peptidase domain containing 1 | Pppde1 | 78825 | 1.94 | 0.00 | 36.70 |
| 1425305_PM_at | zinc finger protein 295 | Zfp295 | 114565 | 2.46 | 0.00 | 29.42 |
| 1426381_PM_at | peroxisome proliferative activated | Pprc1 | 226169 | 1.68 | 0.00 | 12.02 |
| | receptor, gamma, coactivator-related 1 | | | | | |
| 1426645_PM_at | heat shock protein 90, alpha | Hsp90aa1 | 15519 | 1.53 | 0.00 | 4.50 |
| | (cytosolic), class A member 1 | | | | | |
| 1426722_PM_at | solute carrier family 38, member 2 | Slc38a2 | 67760 | 1.78 | 0.00 | 17.22 |
| 1426875_PM_s_at | sulfiredoxin 1 homolog (S. cerevisiae) | Srxn1 | 76650 | 1.51 | 0.00 | 0.18 |
| 1427126_PM_at | heat shock protein 1B | Hspa1b | 15511 | 3.25 | 0.00 | 9.69 |
| 1427127_PM_x_at | heat shock protein 1B | Hspa1b | 15511 | 2.98 | 0.00 | 12.44 |
| 1427912_PM_at | carbonyl reductase 3 | Cbr3 | 109857 | 2.96 | 0.00 | 1.99 |
| 1428400_PM_at | RIKEN cDNA 2200002K05 gene | 2200002K05Rik | 69137 | −1.61 | 0.00 | 4.34 |
| 1428562_PM_at | RIKEN cDNA 2210403K04 gene | 2210403K04Rik | 100042498 | 1.73 | 0.00 | 3.45 |
| 1428654_PM_at | RIKEN cDNA 1200016B10 gene | 1200016B10Rik | 66875 | 1.52 | 0.00 | 15.13 |
| 1428694_PM_at | MIR17 host gene 1 (non-protein | Mir17hg | 75957 | 2.23 | 0.00 | 18.54 |
| | coding) | | | | | |
| 1428834_PM_at | dual specificity phosphatase 4 | Dusp4 | 319520 | 1.93 | 0.00 | 2.47 |
| 1428963_PM_at | RWD domain containing 2A | Rwdd2a | 69519 | 2.10 | 0.00 | 21.23 |
| 1428997_PM_at | PHD finger protein 23 | Phf23 | 78246 | 1.56 | 0.00 | 17.34 |
| 1429056_PM_at | N(alpha)-acetyltransferase 16, NatA | Naa16 | 66897 | 1.56 | 0.00 | 11.32 |
| | auxiliary subunit | | | | | |
| 1429204_PM_at | calcium/calmodulin-dependent | Camk2n2 | | 1.51 | 0.00 | 4.14 |
| | protein kinase II inhibitor 2 | | | | | |
| 1429251_PM_at | hypothetical LOC100503505 /// PR | LOC100503505 | 100503505 | 1.51 | 0.00 | 7.36 |
| | domain containing 2, with ZNF | /// | /// | | | |
| | domain | Prdm2 | 110593 | | | |
| 1429350_PM_at | EP300 interacting inhibitor of | Eid3 | 66341 | 1.64 | 0.00 | 7.61 |
| | differentiation 3 | | | | | |
| 1429863_PM_at | LON peptidase N-terminal domain | Lonrf3 | 74365 | 3.58 | 0.00 | 26.92 |
| | and ring finger 3 | | | | | |
| 1429943_PM_at | chitobiase, di-N-acetyl- | Ctbs | 74245 | −1.51 | 0.00 | 5.73 |
| 1430244_PM_at | RIKEN cDNA 4921509J17 gene | 4921509J17Rik | 70857 | 1.53 | 0.00 | 3.56 |
| 1430392_PM_at | RIKEN cDNA 9530086O07 gene | 9530086O07Rik | 78741 | −1.63 | 0.00 | 4.41 |
| 1430593_PM_at | coiled-coil domain containing 30 | Ccdc30 | 73332 | −1.78 | 0.00 | 2.30 |
| 1430686_PM_at | RIKEN cDNA 4833418N02 gene | 4833418N02Rik | 74597 | −1.50 | 0.00 | 3.61 |
| 1430744_PM_at | napsin A aspartic peptidase | Napsa | 16541 | −4.07 | 0.00 | 34.41 |
| 1430783_PM_at | RIKEN cDNA 4932416J16 gene | 4932416J16Rik | 67541 | −1.64 | 0.00 | 8.70 |
| 1431140_PM_at | thioesterase superfamily member 4 | Them4 | 75778 | 1.62 | 0.00 | 3.78 |
| 1431182_PM_at | heat shock protein 8 /// hypothetical | Hspa8 /// | 15481 | 2.43 | 0.00 | 5.44 |
| | LOC624853 | LOC624853 | /// | | | |
| | | | 624853 | | | |
| 1431254_PM_at | kelch repeat and BTB (POZ) | Kbtbd11 | 74901 | −1.52 | 0.00 | 3.45 |
| | domain containing 11 | | | | | |
| 1431726_PM_a_at | transmembrane protein 80 | Tmem80 | 71448 | −1.52 | 0.00 | 6.79 |
| 1431734_PM_a_at | DnaJ (Hsp40) homolog, subfamily | Dnajb4 | 67035 | 1.98 | 0.00 | 16.02 |
| | B, member 4 | | | | | |
| 1431740_PM_at | solute carrier family 7, (cationic | Slc7a13 | 74087 | −2.14 | 0.00 | 0.21 |
| | amino acid transporter, y+ system) | | | | | |
| | member 13 | | | | | |
| 1432108_PM_at | polycomb group ring finger 6 | Pcgf6 | 71041 | 1.56 | 0.00 | 0.53 |
| 1432600_PM_at | RIKEN cDNA 2310061A09 gene | 2310061A09Rik | 70403 | −1.80 | 0.00 | 3.13 |
| 1432625_PM_at | RIKEN cDNA 5830487K18 gene | 5830487K18Rik | 76125 | −1.92 | 0.00 | 17.35 |
| 1432665_PM_at | RIKEN cDNA 2210416J07 gene | 2210416J07Rik | 72374 | −1.99 | 0.00 | 5.22 |
| 1433398_PM_at | FYVE, RhoGEF and PH domain | Fgd3 | 30938 | 1.60 | 0.00 | 6.72 |
| | containing 3 | | | | | |
| 1433599_PM_at | bromodomain adjacent to zinc | Baz1a | 217578 | 2.12 | 0.00 | 19.42 |
| | finger domain 1A | | | | | |
| 1433674_PM_a_at | small nucleolar RNA host gene | Snhg1 | 83673 | 1.58 | 0.00 | 29.56 |
| | (non-protein coding) 1 | | | | | |
| 1433675_PM_at | small nucleolar RNA host gene | Snhg1 | 83673 | 1.66 | 0.00 | 33.67 |
| | (non-protein coding) 1 | | | | | |
| 1433699_PM_at | tumor necrosis factor, alpha-induced | Tnfaip3 | 21929 | −1.74 | 0.00 | 3.22 |
| | protein 3 | | | | | |
| 1433966_PM_x_at | asparagine synthetase | Asns | 27053 | 2.46 | 0.00 | 13.38 |
| 1434196_PM_at | DnaJ (Hsp40) homolog, subfamily | Dnaja4 | 58233 | 1.51 | 0.00 | 5.67 |
| | A, member 4 | | | | | |
| 1434496_PM_at | polo-like kinase 3 (Drosophila) | Plk3 | 12795 | 3.55 | 0.00 | 37.56 |
| 1434583_PM_at | transmembrane protein 26 | Tmem26 | 327766 | −1.51 | 0.00 | 1.51 |
| 1434660_PM_at | alkB, alkylation repair homolog 1 | Alkbh1 | 211064 | 1.60 | 0.00 | 19.28 |
| | (E. coli) | | | | | |
| 1434901_PM_at | zinc finger and BTB domain | Zbtb2 | 381990 | 2.39 | 0.00 | 35.97 |
| | containing 2 | | | | | |
| 1434967_PM_at | zinc finger, SWIM domain | Zswim6 | 67263 | 1.52 | 0.00 | 10.60 |
| | containing 6 | | | | | |
| 1435005_PM_at | centromere protein E | Cenpe | 229841 | −2.08 | 0.00 | 12.05 |
| 1435035_PM_at | RNA (guanine-9-) methyltransferase | Rg9mtd2 | 108943 | 1.67 | 0.00 | 7.68 |
| | domain containing 2 | | | | | |
| 1435098_PM_at | — | — | — | −1.58 | 0.00 | 5.08 |
| 1435160_PM_at | AHA1, activator of heat shock | Ahsa2 | 268390 | 1.64 | 0.00 | 9.49 |
| | protein ATPase homolog 2 (yeast) | | | | | |
| 1435310_PM_at | synapsin III | Syn3 | 27204 | −2.07 | 0.00 | 1.82 |
| 1435409_PM_at | transmembrane protein 26 | Tmem26 | 327766 | −1.78 | 0.00 | 7.85 |
| 1435632_PM_at | nuclear fragile X mental retardation | Nufip2 | 68564 | 1.52 | 0.00 | 3.20 |
| | protein interacting protein 2 | | | | | |
| 1436200_PM_at | LON peptidase N-terminal domain | Lonrf3 | 74365 | 3.60 | 0.00 | 22.56 |
| | and ring finger 3 | | | | | |
| 1436357_PM_at | predicted gene 10374 | Gm10374 | 100191074 | −1.55 | 0.00 | 12.14 |
| 1436550_PM_at | F-box protein 30 | Fbxo30 | 71865 | 1.55 | 0.00 | 5.34 |
| 1436652_PM_at | RIKEN cDNA 5830418K08 gene | 5830418K08Rik | 319675 | −1.51 | 0.00 | 3.35 |
| 1436725_PM_at | RIKEN cDNA E130306D19 gene | E130306D19Rik | 230098 | −1.66 | 0.00 | 9.92 |
| 1436771_PM_x_at | phosphogluconate dehydrogenase | Pgd | 110208 | 1.50 | 0.00 | 10.26 |
| 1436870_PM_s_at | actin filament associated protein 1- | Afap1l2 | 226250 | −1.62 | 0.00 | 2.06 |
| | like 2 | | | | | |
| 1436974_PM_at | transmembrane protein 88B | Tmem88b | 320587 | −2.68 | 0.00 | 37.59 |
| 1437199_PM_at | dual specificity phosphatase 5 | Dusp5 | 240672 | 4.07 | 0.00 | 32.86 |
| 1437210_PM_a_at | bromodomain containing 2 | Brd2 | 14312 | 1.87 | 0.00 | 25.73 |
| 1437410_PM_at | aldehyde dehydrogenase 2, | Aldh2 | 11669 | 1.75 | 0.00 | 15.95 |
| | mitochondrial | | | | | |
| 1437473_PM_at | avian musculoaponeurotic | Maf | 17132 | −1.56 | 0.00 | 14.85 |
| | fibrosarcoma (v-maf) AS42 | | | | | |
| | oncogene homolog | | | | | |
| 1437884_PM_at | ADP-ribosylation factor-like 5B | Arl5b | 75869 | 1.69 | 0.00 | 5.07 |
| 1438130_PM_at | TAF15 RNA polymerase II, TATA | Taf15 | 70439 | 1.71 | 0.00 | 18.85 |
| | box binding protein (TBP)- | | | | | |
| | associated factor | | | | | |
| 1438133_PM_a_at | cysteine rich protein 61 | Cyr61 | 16007 | 2.19 | 0.00 | 6.73 |
| 1438725_PM_at | mediator complex subunit 13 | Med13 | 327987 | 1.83 | 0.00 | 10.16 |
| 1438764_PM_at | annexin A7 | Anxa7 | 11750 | 1.76 | 0.00 | 6.91 |
| 1438842_PM_at | mitochondrial carrier homolog 2 (C. elegans) | Mtch2 | 56428 | 1.51 | 0.00 | 1.87 |
| 1438880_PM_at | RIKEN cDNA 1700012D14 gene | 1700012D14Rik | 75479 | −1.50 | 0.00 | 5.59 |
| 1438992_PM_x_at | activating transcription factor 4 | Atf4 | 11911 | 1.65 | 0.00 | 17.56 |
| 1439093_PM_at | heat shock protein 4 like | Hspa4l | 18415 | 1.60 | 0.00 | 7.57 |
| 1439094_PM_at | clathrin, heavy polypeptide (Hc) | Cltc | 67300 | 1.89 | 0.00 | 16.19 |
| 1439292_PM_at | — | — | — | 2.02 | 0.00 | 12.91 |
| 1439293_PM_at | cDNA sequence BC031353 | BC031353 | 235493 | −1.67 | 0.00 | 2.08 |
| 1439348_PM_at | S100 calcium binding protein A10 | S100a10 | 20194 | 1.89 | 0.00 | 11.66 |
| | (calpactin) | | | | | |
| 1439352_PM_at | tripartite motif-containing 7 | Trim7 | 94089 | −1.51 | 0.00 | 5.92 |
| 1439640_PM_at | — | — | — | −1.70 | 0.00 | 3.42 |
| 1440076_PM_at | — | — | — | 3.58 | 0.00 | 44.79 |
| 1440222_PM_at | — | — | — | 1.50 | 0.00 | 2.61 |
| 1440255_PM_at | histone H4 transcription factor | Hinfp | 102423 | 1.68 | 0.00 | 12.39 |
| 1440336_PM_at | RIKEN cDNA C130013H08 gene | C130013H08Rik | 100327264 | −1.51 | 0.00 | 2.59 |
| 1440457_PM_at | — | — | — | 1.63 | 0.00 | 4.22 |
| 1440660_PM_at | — | — | — | −1.56 | 0.00 | 5.69 |
| 1440749_PM_at | — | — | — | −1.61 | 0.00 | 0.46 |
| 1440765_PM_at | Fraser syndrome 1 homolog | Fras1 | 231470 | −1.76 | 0.00 | 13.94 |
| | (human) | | | | | |
| 1440779_PM_s_at | solute carrier family 5 | Slc5a9 | 230612 | −1.52 | 0.00 | 2.91 |
| | (sodium/glucose cotransporter), | | | | | |
| | member 9 | | | | | |
| 1440831_PM_at | BTB and CNC homology 1 | Bach1 | 12013 | 1.85 | 0.00 | 35.93 |
| 1440867_PM_at | sprouty homolog 4 (Drosophila) | Spry4 | 24066 | 1.85 | 0.00 | 4.43 |
| 1441042_PM_at | fibroblast growth factor 1 | Fgf1 | 14164 | −2.48 | 0.00 | 21.69 |
| 1441413_PM_at | — | — | — | 1.76 | 0.00 | 11.01 |
| 1441455_PM_at | — | — | — | 1.52 | 0.00 | 6.51 |
| 1441482_PM_at | — | — | — | −2.51 | 0.00 | 10.24 |
| 1441484_PM_at | — | — | — | −1.64 | 0.00 | 3.35 |
| 1441604_PM_at | Esterase D/formylglutathione | Esd | 13885 | 1.57 | 0.00 | 14.39 |
| | hydrolase | | | | | |
| 1441759_PM_at | predicted gene 10804 | Gm10804 | 100038525 | −1.57 | 0.00 | 19.50 |
| 1441794_PM_at | — | — | — | −1.79 | 0.00 | 19.10 |
| 1441973_PM_at | zinc finger protein 295 | Zfp295 | 114565 | 1.82 | 0.00 | 8.47 |
| 1441988_PM_at | protein phosphatase 1K (PP2C | Ppm1k | 243382 | −1.72 | 0.00 | 8.18 |
| | domain containing) | | | | | |
| 1442067_PM_at | — | — | — | −1.67 | 0.00 | 4.73 |
| 1442422_PM_at | — | — | — | 1.70 | 0.00 | 8.67 |
| 1442436_PM_at | fructosamine 3 kinase | Fn3k | 63828 | −1.73 | 0.00 | 5.86 |
| 1442483_PM_at | — | — | — | −1.56 | 0.00 | 0.09 |
| 1442506_PM_at | — | — | — | 5.06 | 0.00 | 33.02 |
| 1442546_PM_at | RIKEN cDNA C730027H18 gene | C730027H18Rik | 319572 | −1.66 | 0.00 | 8.54 |
| 1442633_PM_at | — | — | — | −1.52 | 0.00 | 2.26 |
| 1442671_PM_at | — | — | — | 1.53 | 0.00 | 16.80 |
| 1442726_PM_s_at | expressed sequence AI845619 | AI845619 | 103846 | 5.02 | 0.00 | 37.57 |
| 1442928_PM_at | — | — | — | 1.60 | 0.00 | 8.00 |
| 1442959_PM_at | baculoviral IAP repeat-containing 6 | Birc6 | 12211 | 1.52 | 0.00 | 3.18 |
| 1443049_PM_at | Transmembrane protein 19 | Tmem19 | 67226 | 1.56 | 0.00 | 1.89 |
| 1443054_PM_at | potassium inwardly-rectifying | Kcnj15 | 16516 | −1.51 | 0.00 | 0.20 |
| | channel, subfamily J, member 15 | | | | | |
| 1443100_PM_at | — | — | — | −1.65 | 0.00 | 3.08 |
| 1443109_PM_at | — | — | — | 1.70 | 0.00 | 10.88 |
| 1443159_PM_at | RIKEN cDNA 9130221J17 gene | 9130221J17Rik | 319693 | 8.88 | 0.00 | 53.05 |
| 1443289_PM_at | — | — | — | −1.89 | 0.00 | 5.01 |
| 1443335_PM_at | — | — | — | −2.10 | 0.00 | 11.08 |
| 1443566_PM_at | — | — | — | −1.64 | 0.00 | 0.98 |
| 1443673_PM_x_at | — | — | — | 2.00 | 0.00 | 19.74 |
| 1443897_PM_at | DNA-damage inducible transcript 3 | Ddit3 | 13198 | 3.46 | 0.00 | 25.01 |
| 1443908_PM_at | hypothetical LOC100503447 | LOC100503447 | 100503447 | −1.58 | 0.00 | 7.15 |
| 1444021_PM_at | — | — | — | 3.10 | 0.00 | 33.78 |
| 1444057_PM_at | — | — | — | 1.68 | 0.00 | 19.65 |
| 1444111_PM_at | — | — | — | −1.58 | 0.00 | 14.22 |
| 1444212_PM_at | — | — | — | 1.51 | 0.00 | 7.72 |
| 1444252_PM_at | Predicted gene 15506 | Gm15506 | 100040769 | −1.53 | 0.00 | 2.32 |
| 1444432_PM_at | RIKEN cDNA D330040H18 gene | D330040H18Rik | 320847 | 1.69 | 0.00 | 0.88 |
| 1444722_PM_at | — | — | — | 1.58 | 0.00 | 4.80 |
| 1445032_PM_at | — | — | — | 1.89 | 0.00 | 3.68 |
| 1445089_PM_at | DNA segment, Chr 16, ERATO Doi | D16Ertd778e | 52714 | −2.41 | 0.00 | 9.10 |
| | 778, expressed | | | | | |
| 1445349_PM_at | — | — | — | −1.59 | 0.00 | 3.03 |
| 1445600_PM_at | — | — | — | −1.51 | 0.00 | 1.26 |
| 1445669_PM_at | sprouty homolog 4 (Drosophila) | Spry4 | 24066 | 1.52 | 0.00 | 2.54 |
| 1445857_PM_at | cDNA sequence BC026585 | BC026585 | 226527 | −1.58 | 0.00 | 9.08 |
| 1446075_PM_at | — | — | — | −1.52 | 0.00 | 11.48 |
| 1446172_PM_at | — | — | — | 1.77 | 0.00 | 11.13 |
| 1446175_PM_at | — | — | — | −1.68 | 0.00 | 0.11 |
| 1446336_PM_at | — | — | — | −1.51 | 0.00 | 4.86 |
| 1446621_PM_at | — | — | — | 1.71 | 0.00 | 6.60 |
| 1446921_PM_at | — | — | — | 1.58 | 0.00 | 8.88 |
| 1447172_PM_at | — | — | — | −1.60 | 0.00 | 12.07 |
| 1447396_PM_at | — | — | — | −1.93 | 0.00 | 9.90 |
| 1447411_PM_at | — | — | — | 4.10 | 0.00 | 23.52 |
| 1447930_PM_at | bromodomain adjacent to zinc | Baz1a /// | 100505185 | 2.24 | 0.00 | 17.84 |
| | finger domain 1A /// bromodomain | LOC100505185 | /// | | | |
| | adjacent to zinc finger domain | | 217578 | | | |
| | protein 1A-like | | | | | |
| 1448135_PM_at | activating transcription factor 4 | Atf4 | 11911 | 1.94 | 0.00 | 39.02 |
| 1448239_PM_at | heme oxygenase (decycling) 1 | Hmox1 | 15368 | 5.43 | 0.00 | 7.59 |
| 1448494_PM_at | growth arrest specific 1 | Gas1 | 14451 | −1.57 | 0.00 | 9.47 |
| 1448566_PM_at | solute carrier family 40 (iron- | Slc40a1 | 53945 | 1.57 | 0.00 | 4.57 |
| | regulated transporter), member 1 | | | | | |
| 1448568_PM_a_at | solute carrier family 20, member 1 | Slc20a1 | 20515 | 2.18 | 0.00 | 21.69 |
| 1448694_PM_at | Jun oncogene | Jun | 16476 | 1.58 | 0.00 | 12.53 |
| 1449311_PM_at | BTB and CNC homology 1 | Bach1 | 12013 | 2.26 | 0.00 | 15.31 |
| 1449519_PM_at | growth arrest and DNA-damage- | Gadd45a | 13197 | 1.76 | 0.00 | 8.43 |
| | inducible 45 alpha | | | | | |
| 1450077_PM_at | chromodomain helicase DNA | Chd1 | 12648 | 1.50 | 0.00 | 15.62 |
| | binding protein 1 | | | | | |
| 1450512_PM_at | netrin 4 | Ntn4 | 57764 | −1.57 | 0.00 | 0.97 |
| 1450716_PM_at | a disintegrin-like and | Adamts1 | 11504 | 1.92 | 0.00 | 16.09 |
| | metallopeptidase (reprolysin type) | | | | | |
| | with thrombospondin type 1 motif, 1 | | | | | |
| 1450724_PM_at | family with sequence similarity 126, | Fam126a | 84652 | 1.55 | 0.00 | 14.98 |
| | member A | | | | | |
| 1450957_PM_a_at | sequestosome 1 | Sqstm1 | 18412 | 1.67 | 0.00 | 24.93 |
| 1451083_PM_s_at | alanyl-tRNA synthetase | Aars | 234734 | 1.53 | 0.00 | 12.87 |
| 1451095_PM_at | asparagine synthetase | Asns | 27053 | 2.88 | 0.00 | 16.63 |
| 1451177_PM_at | DnaJ (Hsp40) homolog, subfamily | Dnajb4 | 67035 | 2.32 | 0.00 | 28.09 |
| | B, member 4 | | | | | |
| 1451382_PM_at | ChaC, cation transport regulator-like | Chac1 | 69065 | 30.40 | 0.00 | 47.01 |
| | 1 (E. coli) | | | | | |
| 1451463_PM_at | proline rich 5 (renal) | Prr5 | 109270 | 1.86 | 0.00 | 12.43 |
| 1451516_PM_at | Ras homolog enriched in brain like 1 | Rhebl1 | 69159 | −1.74 | 0.00 | 2.15 |
| 1451612_PM_at | metallothionein 1 | Mt1 | 17748 | 2.47 | 0.00 | 9.16 |
| 1451621_PM_at | PPPDE peptidase domain containing 1 | Pppde1 | 78825 | 1.62 | 0.00 | 11.83 |
| 1451687_PM_a_at | HNF1 homeobox B | Hnf1b | 21410 | −1.56 | 0.00 | 0.13 |
| 1452239_PM_at | gene trap ROSA 26, Philippe | Gt(ROSA)26Sor | 14910 | 1.51 | 0.00 | 13.33 |
| | Soriano | | | | | |
| 1452318_PM_a_at | heat shock protein 1B | Hspa1b | 15511 | 1.65 | 0.00 | 6.11 |
| 1452388_PM_at | heat shock protein 1A | Hspa1a | 193740 | 2.41 | 0.00 | 10.36 |
| 1452623_PM_at | zinc finger protein 759 | Zfp759 | 268670 | −1.67 | 0.00 | 3.57 |
| 1452975_PM_at | alanine-glyoxylate aminotransferase | Agxt2l1 | 71760 | −1.88 | 0.00 | 5.17 |
| | 2-like 1 | | | | | |
| 1453136_PM_at | F-box protein 30 | Fbxo30 | 71865 | 2.01 | 0.00 | 21.95 |
| 1453137_PM_at | F-box protein 30 | Fbxo30 | 71865 | 1.94 | 0.00 | 12.60 |
| 1453374_PM_at | zinc finger, AN1-type domain 5 | Zfand5 | 22682 | 1.67 | 0.00 | 14.03 |
| 1453775_PM_at | RPTOR independent companion of | Rictor | 78757 | 1.66 | 0.00 | 0.89 |
| | MTOR, complex 2 | | | | | |
| 1453828_PM_at | RIKEN cDNA 4932422M17 gene | 4932422M17Rik | 74366 | −1.58 | 0.00 | 5.98 |
| 1454109_PM_a_at | jumonji domain containing 6 | Jmjd6 | 107817 | 2.00 | 0.00 | 16.00 |
| 1454826_PM_at | zinc finger and BTB domain | Zbtb11 | 271377 | 1.53 | 0.00 | 17.14 |
| | containing 11 | | | | | |
| 1455166_PM_at | ADP-ribosylation factor-like 5B | Arl5b | 75869 | 1.64 | 0.00 | 16.25 |
| 1455175_PM_at | PHD finger protein 13 | Phf13 | 230936 | 1.51 | 0.00 | 19.38 |
| 1455387_PM_at | nuclear fragile X mental retardation | Nufip2 | 68564 | 1.61 | 0.00 | 16.27 |
| | protein interacting protein 2 | | | | | |
| 1455454_PM_at | aldo-keto reductase family 1, | Akr1c19 | 432720 | 1.56 | 0.00 | 2.82 |
| | member C19 | | | | | |
| 1455658_PM_at | CGG triplet repeat binding protein 1 | Cggbp1 | 106143 | 1.51 | 0.00 | 3.89 |
| 1455665_PM_at | LON peptidase N-terminal domain | Lonrf1 | 244421 | 1.98 | 0.00 | 18.97 |
| | and ring finger 1 | | | | | |
| 1455959_PM_s_at | glutamate-cysteine ligase, catalytic | Gclc | 14629 | 1.53 | 0.00 | 1.87 |
| | subunit | | | | | |
| 1456070_PM_at | protein tyrosine phosphatase, | Ptprg | 19270 | −2.31 | 0.00 | 3.77 |
| | receptor type, G | | | | | |
| 1456225_PM_x_at | tribbles homolog 3 (Drosophila) | Trib3 | 228775 | 1.56 | 0.00 | 10.36 |
| 1456319_PM_at | — | — | — | −2.06 | 0.00 | 1.62 |
| 1456909_PM_at | glucose-6-phosphate isomerase-like | LOC676974 | 676974 | 1.57 | 0.00 | 7.92 |
| 1456922_PM_at | sorting nexin 29 | Snx29 | 74478 | −1.74 | 0.00 | 15.40 |
| 1456955_PM_at | — | — | — | 1.60 | 0.00 | 23.62 |
| 1457129_PM_at | hypothetical LOC100504796 | LOC100504796 | 100504796 | −1.55 | 0.00 | 1.52 |
| 1457189_PM_at | — | — | — | −1.70 | 0.00 | 7.98 |
| 1457552_PM_at | zinc finger protein 295 | Zfp295 | 114565 | 1.74 | 0.00 | 27.53 |
| 1457586_PM_at | — | — | — | 1.61 | 0.00 | 16.15 |
| 1458096_PM_at | — | — | — | −1.87 | 0.00 | 9.23 |
| 1458176_PM_at | Period homolog 3 (Drosophila) | Per3 | 18628 | −1.57 | 0.00 | 6.04 |
| 1458295_PM_at | cDNA sequence BC038331 | BC038331 | 408056 | −1.57 | 0.00 | 6.16 |
| 1458452_PM_at | Ankyrin repeat domain 11 | Ankrd11 | 77087 | 1.56 | 0.00 | 6.78 |
| 1458503_PM_at | B-cell CLL/lymphoma 7A | Bcl7a | 77045 | −1.54 | 0.00 | 9.28 |
| 1458798_PM_at | — | — | — | −1.56 | 0.00 | 2.17 |
| 1459091_PM_at | — | — | — | 2.78 | 0.00 | 3.27 |
| 1459358_PM_at | — | — | — | 1.56 | 0.00 | 12.39 |
| 1459467_PM_at | expressed sequence AA986715 | AA986715 | 105449 | 1.68 | 0.00 | 17.90 |
| 1459516_PM_at | — | — | — | 1.71 | 0.00 | 8.06 |
| 1459774_PM_at | — | — | — | 1.63 | 0.00 | 8.59 |
| 1459913_PM_at | tumor necrosis factor (ligand) | Tnfsf10 | 22035 | −1.60 | 0.00 | 5.61 |
| | superfamily, member 10 | | | | | |
| 1460033_PM_at | RIKEN cDNA A330023F24 gene | A330023F24Rik | 320977 | 1.50 | 0.00 | 12.94 |
| 1460511_PM_at | plakophilin 2 | Pkp2 | 67451 | 1.67 | 0.00 | 20.15 |

100MMF-vs-Veh, 7 h

| | | | | | | |
|---|---|---|---|---|---|---|
| 1415983_PM_at | lymphocyte cytosolic protein 1 | Lcp1 | 18826 | 1.59 | 0.00 | 8.17 |
| 1416368_PM_at | glutathione S-transferase, alpha 4 | Gsta4 | 14860 | 1.85 | 0.00 | 9.58 |
| 1416563_PM_at | cytidine 5′-triphosphate | Ctps | 51797 | 1.57 | 0.00 | 15.63 |
| | synthase | | | | | |
| 1417700_PM_at | RAB38, member of RAS | Rab38 | 72433 | −1.57 | 0.00 | 10.67 |
| | oncogene family | | | | | |
| 1417884_PM_at | solute carrier family 16 | Slc16a6 | 104681 | 1.55 | 0.00 | 1.62 |
| | (monocarboxylic acid | | | | | |
| | transporters), member 6 | | | | | |
| 1417932_PM_at | interleukin 18 | Il18 | 16173 | −1.52 | 0.00 | 0.25 |
| 1418174_PM_at | D site albumin promoter | Dbp | 13170 | −1.73 | 0.00 | 0.84 |
| | binding protein | | | | | |
| 1418358_PM_at | sperm mitochondria-associated | Smcp | 17235 | −1.96 | 0.00 | 3.15 |
| | cysteine-rich protein | | | | | |
| 1418571_PM_at | tumor necrosis factor receptor | Tnfrsf12a | 27279 | 2.40 | 0.00 | 11.11 |
| | superfamily, member 12a | | | | | |
| 1418572_PM_x_at | tumor necrosis factor receptor | Tnfrsf12a | 27279 | 2.45 | 0.00 | 11.74 |
| | superfamily, member 12a | | | | | |
| 1418601_PM_at | aldehyde dehydrogenase family | Aldh1a7 | 26358 | 2.16 | 0.00 | 4.53 |
| | 1, subfamily A7 | | | | | |
| 1418627_PM_at | glutamate-cysteine ligase, | Gclm | 14630 | 1.64 | 0.00 | 13.50 |
| | modifier subunit | | | | | |
| 1418949_PM_at | growth differentiation factor 15 | Gdf15 | 23886 | 1.98 | 0.00 | 4.89 |
| 1419086_PM_at | fibroblast growth factor binding | Fgfbp1 | 14181 | 1.73 | 0.00 | 3.06 |
| | protein 1 | | | | | |
| 1419253_PM_at | methylenetetrahydrofolate | Mthfd2 | 17768 | 1.89 | 0.00 | 8.51 |
| | dehydrogenase (NAD+ | | | | | |
| | dependent), | | | | | |
| | methenyltetrahydrofolate | | | | | |
| | cyclohydrolase | | | | | |
| 1419254_PM_at | methylenetetrahydrofolate | Mthfd2 | 17768 | 1.68 | 0.00 | 12.97 |
| | dehydrogenase (NAD+ | | | | | |
| | dependent), | | | | | |
| | methenyltetrahydrofolate | | | | | |
| | cyclohydrolase | | | | | |
| 1419435_PM_at | aldehyde oxidase 1 | Aox1 | 11761 | 2.50 | 0.00 | 7.22 |
| 1419942_PM_at | Sulfiredoxin 1 homolog (S. cerevisiae) | Srxn1 | 76650 | 2.45 | 0.00 | 18.17 |
| 1420696_PM_at | sema domain, immunoglobulin | Sema3c | 20348 | 1.66 | 0.00 | 2.79 |
| | domain (Ig), short basic | | | | | |
| | domain, secreted, (semaphorin) | | | | | |
| | 3C | | | | | |
| 1421041_PM_s_at | predicted gene 3776 /// | Gm3776 /// | 100042295 | 1.63 | 0.00 | 11.91 |
| | glutathione S-transferase, alpha | Gsta1 /// Gsta2 | /// | | | |
| | 1 (Ya) /// glutathione S- | | 14857 /// | | | |
| | transferase, alpha 2 (Yc2) | | 14858 | | | |
| 1421209_PM_s_at | inhibitor of kappaB kinase | Ikbkg | 16151 | 1.77 | 0.00 | 13.26 |
| | gamma | | | | | |
| 1421529_PM_a_at | thioredoxin reductase 1 | Txnrd1 | 50493 | 1.72 | 0.00 | 20.64 |
| 1421609_PM_a_at | camello-like 3 | Cml3 | 93674 | −1.64 | 0.00 | 13.47 |
| 1421816_PM_at | glutathione reductase | Gsr | 14782 | 1.83 | 0.00 | 0.21 |
| 1422327_PM_s_at | glucose-6-phosphate | G6pd2 /// G6pdx | 14380 /// | 1.86 | 0.00 | 14.27 |
| | dehydrogenase 2 /// glucose-6- | | 14381 | | | |
| | phosphate dehydrogenase X- | | | | | |
| | linked | | | | | |
| 1422533_PM_at | cytochrome P450, family 51 | Cyp51 | 13121 | −1.54 | 0.00 | 11.86 |
| 1422534_PM_at | cytochrome P450, family 51 | Cyp51 | 13121 | −1.59 | 0.00 | 1.35 |
| 1422557_PM_s_at | metallothionein 1 | Mt1 | 17748 | 1.89 | 0.00 | 14.23 |
| 1423186_PM_at | T-cell lymphoma invasion and | Tiam2 | 24001 | 2.27 | 0.00 | 16.25 |
| | metastasis 2 | | | | | |
| 1423413_PM_at | N-myc downstream regulated | Ndrg1 | 17988 | 1.52 | 0.00 | 3.57 |
| | gene 1 | | | | | |
| 1423436_PM_at | glutathione S-transferase, alpha 3 | Gsta3 | 14859 | 1.99 | 0.00 | 18.13 |
| 1423437_PM_at | glutathione S-transferase, alpha 3 | Gsta3 | 14859 | 2.95 | 0.00 | 19.68 |
| 1423627_PM_at | NAD(P)H dehydrogenase, | Nqo1 | 18104 | 1.96 | 0.00 | 23.24 |
| | quinone 1 | | | | | |
| 1423706_PM_a_at | phosphogluconate | Pgd | 110208 | 2.21 | 0.00 | 24.15 |
| | dehydrogenase | | | | | |
| 1423723_PM_s_at | TAR DNA binding protein | Tardbp | 230908 | 1.62 | 0.00 | 14.92 |
| 1424126_PM_at | aminolevulinic acid synthase 1 | Alas1 | 11655 | 1.51 | 0.00 | 0.85 |
| 1424296_PM_at | glutamate-cysteine ligase, | Gclc | 14629 | 1.87 | 0.00 | 4.58 |
| | catalytic subunit | | | | | |
| 1424486_PM_a_at | thioredoxin reductase 1 | Txnrd1 | 50493 | 3.08 | 0.00 | 6.85 |
| 1424487_PM_x_at | thioredoxin reductase 1 | Txnrd1 | 50493 | 2.37 | 0.00 | 8.59 |
| 1424969_PM_s_at | uridine phosphorylase 2 | Upp2 | 76654 | −3.63 | 0.00 | 11.38 |
| 1425009_PM_at | t-complex-associated testis | Tcte2 | 21646 | −1.61 | 0.00 | 3.90 |
| | expressed 2 | | | | | |
| 1425351_PM_at | sulfiredoxin 1 homolog (S. cerevisiae) | Srxn1 | 76650 | 2.92 | 0.00 | 16.23 |
| 1425743_PM_at | tripartite motif-containing 7 | Trim7 | 94089 | −1.59 | 0.00 | 4.82 |
| 1425778_PM_at | indoleamine 2,3-dioxygenase 2 | Ido2 | 209176 | −1.52 | 0.00 | 6.47 |
| 1426230_PM_at | sphingosine kinase 2 | Sphk2 | 56632 | −1.54 | 0.00 | 0.51 |
| 1426275_PM_a_at | UDP-glucuronate | Uxs1 | 67883 | 1.50 | 0.00 | 4.48 |
| | decarboxylase 1 | | | | | |
| 1426300_PM_at | activated leukocyte cell | Alcam | 11658 | 2.66 | 0.00 | 24.34 |
| | adhesion molecule | | | | | |
| 1426301_PM_at | activated leukocyte cell | Alcam | 11658 | 2.09 | 0.00 | 17.66 |
| | adhesion molecule | | | | | |
| 1426399_PM_at | von Willebrand factor A | Vwa1 | 246228 | −1.69 | 0.00 | 8.45 |
| | domain containing 1 | | | | | |
| 1426645_PM_at | heat shock protein 90, alpha | Hsp90aa1 | 15519 | 1.50 | 0.00 | 3.50 |
| | (cytosolic), class A member 1 | | | | | |
| 1426767_PM_at | WD repeat domain 90 | Wdr90 | 106618 | −1.58 | 0.00 | 6.02 |
| 1426875_PM_s_at | sulfiredoxin 1 homolog (S. cerevisiae) | Srxn1 | 76650 | 2.29 | 0.00 | 17.13 |
| 1426887_PM_at | nudix (nucleoside diphosphate | Nudt11 | 58242 | 1.62 | 0.00 | 10.47 |
| | linked moiety X)-type motif 11 | | | | | |
| 1427123_PM_s_at | coatomer protein complex, | Copg2as2 | 100044236 | 1.56 | 0.00 | 8.41 |
| | subunit gamma 2, antisense 2 | | | | | |
| 1427347_PM_s_at | tubulin, beta 2A | Tubb2a | 22151 | 1.52 | 0.00 | 12.89 |
| 1427912_PM_at | carbonyl reductase 3 | Cbr3 | 109857 | 9.54 | 0.00 | 23.01 |
| 1427932_PM_s_at | RIKEN cDNA 1200003I10 | 1200003I10Rik | 319269 | 1.63 | 0.00 | 14.40 |
| | gene /// RIKEN cDNA | /// | /// | | | |
| | 1200015M12 gene /// RIKEN | 1200015M12Rik | 319443 | | | |
| | cDNA A130040M12 gene /// | /// | /// 71719 | | | |
| | RIKEN cDNA E430024C06 | A130040M12Rik | /// 71739 | | | |
| | gene | /// | | | | |
| | | E430024C06Rik | | | | |
| 1428223_PM_at | major facilitator superfamily | Mfsd2a | 76574 | −3.38 | 0.00 | 4.98 |
| | domain containing 2A | | | | | |
| 1428427_PM_at | F-box and leucine-rich repeat | Fbxl2 | 72179 | −1.68 | 0.00 | 2.67 |
| | protein 2 | | | | | |
| 1428758_PM_at | transmembrane protein 86A | Tmem86a | 67893 | −1.56 | 0.00 | 3.45 |
| 1428942_PM_at | metallothionein 2 | Mt2 | 17750 | 2.71 | 0.00 | 10.91 |
| 1429001_PM_at | pirin | Pir | 69656 | 2.63 | 0.00 | 23.41 |
| 1429262_PM_at | Ras association (RalGDS/AF- | Rassf6 | 73246 | 1.65 | 0.00 | 18.06 |
| | 6) domain family member 6 | | | | | |
| 1430111_PM_a_at | branched chain | Bcat1 | 12035 | −1.59 | 0.00 | 0.65 |
| | aminotransferase 1, cytosolic | | | | | |
| 1430135_PM_at | deoxyribonuclease II alpha | Dnase2a | 13423 | 2.20 | 0.00 | 17.74 |
| 1430530_PM_s_at | NmrA-like family domain | Nmral1 | 67824 | −1.52 | 0.00 | 7.09 |
| | containing 1 | | | | | |
| 1430744_PM_at | napsin A aspartic peptidase | Napsa | 16541 | −1.79 | 0.00 | 3.64 |
| 1433966_PM_x_at | asparagine synthetase | Asns | 27053 | 2.13 | 0.00 | 8.32 |
| 1434456_PM_at | RUN domain containing 3B | Rundc3b | 242819 | 1.54 | 0.00 | 2.06 |
| 1434716_PM_at | hepatitis A virus cellular | Havcr1 | 171283 | 3.22 | 0.00 | 4.40 |
| | receptor 1 | | | | | |
| 1434775_PM_at | par-3 (partitioning defective 3) | Pard3 | 93742 | 1.53 | 0.00 | 3.67 |
| | homolog (C. elegans) | | | | | |
| 1434919_PM_at | alkylglycerone phosphate | Agps | 228061 | −1.68 | 0.00 | 12.45 |
| | synthase | | | | | |
| 1434976_PM_x_at | eukaryotic translation initiation | Eif4ebp1 | 13685 | 1.56 | 0.00 | 7.40 |
| | factor 4E binding protein 1 | | | | | |
| 1434990_PM_at | protein phosphatase 1E (PP2C | Ppm1e | 320472 | −1.53 | 0.00 | 5.24 |
| | domain containing) | | | | | |
| 1435005_PM_at | centromere protein E | Cenpe | 229841 | −1.60 | 0.00 | 1.68 |
| 1435188_PM_at | predicted gene 129 | Gm129 | 229599 | −2.12 | 0.00 | 0.27 |
| 1435311_PM_s_at | synapsin III | Syn3 | 27204 | −1.68 | 0.00 | 2.11 |
| 1435646_PM_at | inhibitor of kappaB kinase | Ikbkg | 16151 | 1.80 | 0.00 | 12.18 |
| | gamma | | | | | |
| 1435918_PM_at | family with sequence similarity | Fam107a | 268709 | −1.77 | 0.00 | 2.52 |
| | 107, member A | | | | | |
| 1436101_PM_at | ring finger protein 24 | Rnf24 | 51902 | −2.03 | 0.00 | 10.03 |
| 1436210_PM_at | glycerol kinase 5 (putative) | Gk5 | 235533 | 1.55 | 0.00 | 3.37 |
| 1436771_PM_x_at | phosphogluconate | Pgd | 110208 | 1.87 | 0.00 | 26.21 |
| | dehydrogenase | | | | | |
| 1436791_PM_at | wingless-related MMTV | Wnt5a | 22418 | 1.69 | 0.00 | 14.95 |
| | integration site 5A | | | | | |
| 1437199_PM_at | dual specificity phosphatase 5 | Dusp5 | 240672 | 1.74 | 0.00 | 2.24 |
| 1437380_PM_x_at | phosphogluconate | Pgd | 110208 | 1.87 | 0.00 | 25.07 |
| | dehydrogenase | | | | | |
| 1437467_PM_at | activated leukocyte cell | Alcam | 11658 | 2.00 | 0.00 | 26.56 |
| | adhesion molecule | | | | | |
| 1438211_PM_s_at | D site albumin promoter | Dbp | 13170 | −1.63 | 0.00 | 1.17 |
| | binding protein | | | | | |
| 1438627_PM_x_at | phosphogluconate | Pgd | 110208 | 1.78 | 0.00 | 20.84 |
| | dehydrogenase | | | | | |
| 1439332_PM_at | DNA-damage-inducible | Ddit4l | 73284 | 1.83 | 0.00 | 11.60 |
| | transcript 4-like | | | | | |
| 1439352_PM_at | tripartite motif-containing 7 | Trim7 | 94089 | −1.55 | 0.00 | 7.36 |
| 1439695_PM_a_at | kinesin family member 20B | Kif20b | 240641 | −2.61 | 0.00 | 2.30 |
| 1439866_PM_at | cullin 9 | Cul9 | 78309 | −1.52 | 0.00 | 6.47 |
| 1440330_PM_at | Histone cluster 1, H2be | Hist1h2be | 319179 | 1.98 | 0.00 | 0.34 |
| 1440882_PM_at | low density lipoprotein | Lrp8 | 16975 | 2.19 | 0.00 | 1.98 |
| | receptor-related protein 8, | | | | | |
| | apolipoprotein e receptor | | | | | |
| 1441042_PM_at | fibroblast growth factor 1 | Fgf1 | 14164 | −1.71 | 0.00 | 5.44 |
| 1441944_PM_s_at | G protein-coupled receptor 135 | Gpr135 | 238252 | 1.66 | 0.00 | 13.40 |
| 1442018_PM_at | B-cell translocation gene 1, | Btg1 | 12226 | 1.60 | 0.00 | 2.05 |
| | anti-proliferative | | | | | |
| 1442051_PM_at | histone cluster 2, H3c1 | Hist2h3c1 | 15077 | 1.65 | 0.00 | 4.14 |
| 1442145_PM_at | ATPase type 13A3 | Atp13a3 | 224088 | 1.52 | 0.00 | 12.29 |
| 1442291_PM_at | lysophosphatidic acid receptor 2 | Lpar2 | 53978 | 1.84 | 0.00 | 5.17 |
| 1443870_PM_at | ATP-binding cassette, sub- | Abcc4 | 239273 | 2.09 | 0.00 | 22.38 |
| | family C (CFTR/MRP), | | | | | |
| | member 4 | | | | | |
| 1444139_PM_at | DNA-damage-inducible | Ddit4l | 73284 | 1.85 | 0.00 | 12.51 |
| | transcript 4-like | | | | | |
| 1444265_PM_at | — | — | — | 1.53 | 0.00 | 1.15 |
| 1444432_PM_at | RIKEN cDNA D330040H18 | D330040H18Rik | 320847 | 1.77 | 0.00 | 2.38 |
| | gene | | | | | |
| 1444682_PM_at | cDNA Sequence BC037032 | BC037032 | 414066 | 1.69 | 0.00 | 4.90 |
| 1445089_PM_at | DNA segment, Chr 16, ERATO | D16Ertd778e | 52714 | −1.87 | 0.00 | 1.81 |
| | Doi 778, expressed | | | | | |
| 1445668_PM_at | — | — | — | 1.55 | 0.00 | 4.07 |
| 1447396_PM_at | — | — | — | −1.73 | 0.00 | 5.41 |
| 1448160_PM_at | lymphocyte cytosolic protein 1 | Lcp1 | 18826 | 1.56 | 0.00 | 2.67 |
| 1448239_PM_at | heme oxygenase (decycling) 1 | Hmox1 | 15368 | 7.48 | 0.00 | 12.43 |
| 1448354_PM_at | glucose-6-phosphate | G6pdx | 14381 | 1.75 | 0.00 | 19.44 |
| | dehydrogenase X-linked | | | | | |
| 1448566_PM_at | solute carrier family 40 (iron- | Slc40a1 | 53945 | 1.51 | 0.00 | 3.04 |
| | regulated transporter), member 1 | | | | | |
| 1448568_PM_a_at | solute carrier family 20, | Slc20a1 | 20515 | 1.70 | 0.00 | 8.63 |
| | member 1 | | | | | |
| 1448700_PM_at | G0/G1 switch gene 2 | G0s2 | 14373 | 1.50 | 0.00 | 2.11 |
| 1448766_PM_at | gap junction protein, beta 1 | Gjb1 | 14618 | 1.50 | 0.00 | 4.82 |
| 1448818_PM_at | wingless-related MMTV | Wnt5a | 22418 | 1.81 | 0.00 | 8.49 |
| | integration site 5A | | | | | |
| 1448894_PM_at | aldo-keto reductase family 1, | Akr1b8 | 14187 | 1.86 | 0.00 | 5.45 |
| | member B8 | | | | | |
| 1449220_PM_at | GTPase, IMAP family member 3 | Gimap3 | 83408 | −1.51 | 0.00 | 0.01 |
| 1449937_PM_at | endonuclease, polyU-specific | Endou | 19011 | 1.52 | 0.00 | 1.08 |
| 1450410_PM_a_at | solute carrier family 48 (heme | Slc48a1 | 67739 | 1.58 | 0.00 | 20.61 |
| | transporter), member 1 | | | | | |
| 1450702_PM_at | hemochromatosis | Hfe | 15216 | 1.57 | 0.00 | 13.60 |
| 1450869_PM_at | fibroblast growth factor 1 | Fgf1 | 14164 | −1.76 | 0.00 | 19.40 |
| 1450871_PM_a_at | branched chain | Bcat1 | 12035 | −1.83 | 0.00 | 10.55 |
| | aminotransferase 1, cytosolic | | | | | |
| 1450957_PM_a_at | sequestosome 1 | Sqstm1 | 18412 | 1.62 | 0.00 | 22.05 |
| 1450977_PM_s_at | N-myc downstream regulated | Ndrg1 | 17988 | 1.54 | 0.00 | 4.35 |
| | gene 1 | | | | | |
| 1451041_PM_at | Rho-associated coiled-coil | Rock2 | 19878 | 1.60 | 0.00 | 8.99 |
| | containing protein kinase 2 | | | | | |
| 1451095_PM_at | asparagine synthetase | Asns | 27053 | 2.23 | 0.00 | 8.17 |
| 1451386_PM_at | biliverdin reductase B (flavin | Blvrb | 233016 | 1.83 | 0.00 | 16.26 |
| | reductase (NADPH)) | | | | | |
| 1451548_PM_at | uridine phosphorylase 2 | Upp2 | 76654 | −4.00 | 0.00 | 10.32 |
| 1451680_PM_at | sulfiredoxin 1 homolog (S. cerevisiae) | Srxn1 | 76650 | 2.68 | 0.00 | 15.66 |
| 1451751_PM_at | DNA-damage-inducible | Ddit4l | 73284 | 2.62 | 0.00 | 14.99 |
| | transcript 4-like | | | | | |
| 1451828_PM_a_at | acyl-CoA synthetase long-chain | Acsl4 | 50790 | 1.78 | 0.00 | 8.74 |
| | family member 4 | | | | | |
| 1452733_PM_at | pantothenate kinase 2 | Pank2 | 74450 | −1.94 | 0.00 | 11.78 |
| 1452975_PM_at | alanine-glyoxylate | Agxt2l1 | 71760 | −1.74 | 0.00 | 2.48 |
| | aminotransferase 2-like 1 | | | | | |
| 1453234_PM_at | RIKEN cDNA 1300002K09 | 1300002K09Rik | 74152 | 1.98 | 0.00 | 8.39 |
| | gene | | | | | |
| 1453474_PM_at | abhydrolase domain containing | Abhd15 | 67477 | 1.64 | 0.00 | 6.58 |
| | 15 | | | | | |
| 1454690_PM_at | inhibitor of kappaB kinase | Ikbkg | 16151 | 1.61 | 0.00 | 23.68 |
| | gamma | | | | | |
| 1454865_PM_at | solute carrier family 9 | Slc9a8 | 77031 | −1.68 | 0.00 | 8.56 |
| | (sodium/hydrogen exchanger), | | | | | |
| | member 8 | | | | | |
| 1454992_PM_at | solute carrier family 7 (cationic | Slc7a1 | 11987 | 1.62 | 0.00 | 5.64 |
| | amino acid transporter, y+ | | | | | |
| | system), member 1 | | | | | |
| 1455454_PM_at | aldo-keto reductase family 1, | Akr1c19 | 432720 | 1.97 | 0.00 | 13.38 |
| | member C19 | | | | | |
| 1455699_PM_at | branched chain | Bcat1 | 12035 | −2.29 | 0.00 | 4.03 |
| | aminotransferase 1, cytosolic | | | | | |
| 1455902_PM_x_at | Ras homolog gene family, | Rhof | 23912 | 1.66 | 0.00 | 4.72 |
| | member f | | | | | |
| 1455959_PM_s_at | glutamate-cysteine ligase, | Gclc | 14629 | 1.81 | 0.00 | 9.06 |
| | catalytic subunit | | | | | |
| 1456509_PM_at | RIKEN cDNA 1110032F04 | 1110032F04Rik | 68725 | 4.65 | 0.00 | 13.18 |
| | gene | | | | | |
| 1456524_PM_at | neuregulin 1 | Nrg1 | 211323 | 1.81 | 0.00 | 7.45 |
| 1456888_PM_at | 6-phosphofructo-2- | Pfkfb4 | 270198 | 1.69 | 0.00 | 5.52 |
| | kinase/fructose-2,6- | | | | | |
| | biphosphatase 4 | | | | | |
| 1457038_PM_at | Fras1 related extracellular | Frem2 | 242022 | −1.66 | 0.00 | 1.18 |
| | matrix protein 2 | | | | | |
| 1457110_PM_at | pantothenate kinase 1 | Pank1 | 75735 | −1.79 | 0.00 | 7.18 |
| 1457594_PM_at | — | — | — | −1.52 | 0.00 | 0.30 |
| 1457707_PM_at | multiple C2 domains, | Mctp2 | 244049 | 1.54 | 0.00 | 5.47 |
| | transmembrane 2 | | | | | |
| 1459091_PM_at | — | — | — | 3.52 | 0.00 | 7.85 |
| 1459274_PM_at | G protein-coupled receptor 135 | Gpr135 | 238252 | 1.53 | 0.00 | 2.74 |
| 1460196_PM_at | carbonyl reductase 1 | Cbr1 | 12408 | 1.58 | 0.00 | 9.82 |
| 1460483_PM_at | RIKEN cDNA 2610034E01 | 2610034E01Rik | 69236 | 1.56 | 0.00 | 2.54 |
| | gene | | | | | |
| 1460591_PM_at | estrogen receptor 1 (alpha) | Esr1 | 13982 | −1.54 | 0.00 | 11.67 |
| 1460632_PM_at | retinol dehydrogenase 10 (all- | Rdh10 | 98711 | 1.68 | 0.00 | 6.32 |
| | trans) | | | | | |

100MMF-vs-Veh, 12 h

| | | | | | | |
|---|---|---|---|---|---|---|
| 1416368_PM_at | glutathione S-transferase, | Gsta4 | 14860 | 1.95 | 0.00 | 12.75 |
| | alpha 4 | | | | | |
| 1416468_PM_at | aldehyde dehydrogenase | Aldh1a1 | 11668 | 2.21 | 0.00 | 2.63 |
| | family 1, subfamily A1 | | | | | |
| 1417150_PM_at | solute carrier family 6 | Slc6a4 | 15567 | 1.58 | 0.00 | 0.15 |
| | (neurotransmitter | | | | | |
| | transporter, serotonin), | | | | | |
| | member 4 | | | | | |
| 1418215_PM_at | meprin 1 beta | Mep1b | 17288 | −1.55 | 0.00 | 17.06 |
| 1418358_PM_at | sperm mitochondria- | Smcp | 17235 | −2.49 | 0.00 | 10.89 |
| | associated cysteine-rich | | | | | |
| | protein | | | | | |
| 1418601_PM_at | aldehyde dehydrogenase | Aldh1a7 | 26358 | 3.02 | 0.00 | 15.12 |
| | family 1, subfamily A7 | | | | | |
| 1418649_PM_at | EGL nine homolog 3 (C. elegans) | Egln3 | 112407 | 1.71 | 0.00 | 15.00 |
| 1418706_PM_at | solute carrier family 38, | Slc38a3 | 76257 | 1.81 | 0.00 | 7.56 |
| | member 3 | | | | | |
| 1418746_PM_at | paroxysmal | Pnkd | 56695 | 1.51 | 0.00 | 15.01 |
| | nonkinesiogenic | | | | | |
| | dyskinesia | | | | | |
| 1418847_PM_at | arginase type II | Arg2 | 11847 | 2.70 | 0.00 | 12.26 |
| 1418949_PM_at | growth differentiation | Gdf15 | 23886 | 2.01 | 0.00 | 5.86 |
| | factor 15 | | | | | |
| 1419253_PM_at | methylenetetrahydrofolate | Mthfd2 | 17768 | 1.65 | 0.00 | 3.73 |
| | dehydrogenase (NAD+ | | | | | |
| | dependent), | | | | | |
| | methenyltetrahydrofolate | | | | | |
| | cyclohydrolase | | | | | |
| 1419435_PM_at | aldehyde oxidase 1 | Aox1 | 11761 | 2.23 | 0.00 | 4.77 |
| 1419754_PM_at | myosin VA | Myo5a | 17918 | −3.09 | 0.00 | 27.51 |
| 1419942_PM_at | Sulfiredoxin 1 homolog | Srxn1 | 76650 | 1.96 | 0.00 | 9.74 |
| | (S. cerevisiae) | | | | | |
| 1420654_PM_a_at | glucan (1,4-alpha-), | Gbe1 | 74185 | 1.51 | 0.00 | 9.90 |
| | branching enzyme 1 | | | | | |
| 1421040_PM_a_at | glutathione S-transferase, | Gsta2 | 14858 | 1.61 | 0.00 | 21.37 |
| | alpha 2 (Yc2) | | | | | |
| 1421041_PM_s_at | predicted gene 3776 /// | Gm3776 /// | 100042295 | 1.68 | 0.00 | 14.44 |
| | glutathione S-transferase, | Gsta1 /// Gsta2 | /// 14857 | | | |
| | alpha 1 (Ya) /// | | /// 14858 | | | |
| | glutathione S-transferase, | | | | | |
| | alpha 2 (Yc2) | | | | | |
| 1421167_PM_at | ATPase, class VI, type | Atp11a | 50770 | −1.62 | 0.00 | 18.30 |
| | 11A | | | | | |
| 1421209_PM_s_at | inhibitor of kappaB kinase | Ikbkg | 16151 | 1.57 | 0.00 | 7.26 |
| | gamma | | | | | |
| 1421529_PM_a_at | thioredoxin reductase 1 | Txnrd1 | 50493 | 1.55 | 0.00 | 13.72 |
| 1421756_PM_a_at | G protein-coupled | Gpr19 | 14760 | 1.50 | 0.00 | 3.13 |
| | receptor 19 | | | | | |
| 1422327_PM_s_at | glucose-6-phosphate | G6pd2 /// G6pdx | 14380 /// | 1.91 | 0.00 | 16.53 |
| | dehydrogenase 2 /// | | 14381 | | | |
| | glucose-6-phosphate | | | | | |
| | dehydrogenase X-linked | | | | | |
| 1422533_PM_at | cytochrome P450, family | Cyp51 | 13121 | −1.95 | 0.00 | 29.96 |
| | 51 | | | | | |
| 1422534_PM_at | cytochrome P450, family | Cyp51 | 13121 | −1.79 | 0.00 | 6.44 |
| | 51 | | | | | |
| 1422606_PM_at | C1q and tumor necrosis | C1qtnf3 | 81799 | −1.57 | 0.00 | 3.54 |
| | factor related protein 3 | | | | | |
| 1422645_PM_at | hemochromatosis | Hfe | 15216 | 1.56 | 0.00 | 19.78 |
| 1422966_PM_a_at | transferrin receptor | Tfrc | 22042 | 1.51 | 0.00 | 4.27 |
| 1422997_PM_s_at | acyl-CoA thioesterase 1 | Acot1 /// Acot2 | 171210 /// | 1.51 | 0.00 | 2.52 |
| | /// acyl-CoA thioesterase 2 | | 26897 | | | |
| 1423186_PM_at | T-cell lymphoma invasion | Tiam2 | 24001 | 1.84 | 0.00 | 8.14 |
| | and metastasis 2 | | | | | |
| 1423436_PM_at | glutathione S-transferase, | Gsta3 | 14859 | 1.95 | 0.00 | 18.07 |
| | alpha 3 | | | | | |
| 1423437_PM_at | glutathione S-transferase, | Gsta3 | 14859 | 2.85 | 0.00 | 19.42 |
| | alpha 3 | | | | | |
| 1423596_PM_at | NIMA (never in mitosis | Nek6 | 59126 | 1.69 | 0.00 | 10.67 |
| | gene a)-related expressed | | | | | |
| | kinase 6 | | | | | |
| 1423627_PM_at | NAD(P)H dehydrogenase, | Nqo1 | 18104 | 2.11 | 0.00 | 29.37 |
| | quinone 1 | | | | | |
| 1423706_PM_a_at | phosphogluconate | Pgd | 110208 | 2.27 | 0.00 | 26.93 |
| | dehydrogenase | | | | | |
| 1423954_PM_at | complement component 3 | C3 | 12266 | 2.34 | 0.00 | 10.44 |
| 1424126_PM_at | aminolevulinic acid | Alas1 | 11655 | 2.02 | 0.00 | 13.98 |
| | synthase 1 | | | | | |
| 1424279_PM_at | fibrinogen alpha chain | Fga | 14161 | 1.79 | 0.00 | 0.10 |
| 1424487_PM_x_at | thioredoxin reductase 1 | Txnrd1 | 50493 | 1.74 | 0.00 | 0.40 |
| 1424626_PM_at | RIKEN cDNA | 2010003K11Rik | 69861 | 1.65 | 0.00 | 4.69 |
| | 2010003K11 gene | | | | | |
| 1424638_PM_at | cyclin-dependent kinase | Cdkn1a | 12575 | 2.73 | 0.00 | 7.64 |
| | inhibitor 1A (P21) | | | | | |
| 1424835_PM_at | glutathione S-transferase, | Gstm4 | 14865 | 1.70 | 0.00 | 22.19 |
| | mu 4 | | | | | |
| 1425351_PM_at | sulfiredoxin 1 homolog | Srxn1 | 76650 | 1.89 | 0.00 | 3.09 |
| | (S. cerevisiae) | | | | | |
| 1425627_PM_x_at | glutathione S-transferase, | Gstm1 | 14862 | 1.56 | 0.00 | 29.78 |
| | mu 1 | | | | | |
| 1426047_PM_a_at | protein tyrosine | Ptprr | 19279 | 1.69 | 0.00 | 7.66 |
| | phosphatase, receptor | | | | | |
| | type, R | | | | | |
| 1426300_PM_at | activated leukocyte cell | Alcam | 11658 | 2.83 | 0.00 | 28.33 |
| | adhesion molecule | | | | | |
| 1426301_PM_at | activated leukocyte cell | Alcam | 11658 | 2.09 | 0.00 | 18.49 |
| | adhesion molecule | | | | | |
| 1426502_PM_s_at | glutamic pyruvic | Gpt | 76282 | 1.51 | 0.00 | 6.29 |
| | transaminase, soluble | | | | | |
| 1426875_PM_s_at | sulfiredoxin 1 homolog | Srxn1 | 76650 | 1.91 | 0.00 | 9.94 |
| | (S. cerevisiae) | | | | | |
| 1427224_PM_at | acyl-CoA synthetase | Acsm2 | 233799 | −1.52 | 0.00 | 0.31 |
| | medium-chain family | | | | | |
| | member 2 | | | | | |
| 1427912_PM_at | carbonyl reductase 3 | Cbr3 | 109857 | 7.02 | 0.00 | 18.24 |
| 1427932_PM_s_at | RIKEN cDNA | 1200003I10Rik | 319269 /// | 1.54 | 0.00 | 11.48 |
| | 1200003I10 gene /// | /// | 319443 /// | | | |
| | RIKEN cDNA | 1200015M12Rik | 71719 /// | | | |
| | 1200015M12 gene /// | /// | 71739 | | | |
| | RIKEN cDNA | A130040M12Rik | | | | |
| | A130040M12 gene /// | /// | | | | |
| | RIKEN cDNA | E430024C06Rik | | | | |
| | E430024C06 gene | | | | | |
| 1428012_PM_at | complement component 8, | C8a | 230558 | −1.51 | 0.00 | 4.84 |
| | alpha polypeptide | | | | | |
| 1428023_PM_at | RIKEN cDNA | 3110009E18Rik | 73103 | 1.58 | 0.00 | 3.27 |
| | 3110009E18 gene | | | | | |
| 1428343_PM_at | REST corepressor 3 | Rcor3 | 214742 | −1.51 | 0.00 | 1.51 |
| 1428988_PM_at | ATP-binding cassette, | Abcc3 | 76408 | 1.64 | 0.00 | 0.82 |
| | sub-family C | | | | | |
| | (CFTR/MRP), member 3 | | | | | |
| 1429001_PM_at | pirin | Pir | 69656 | 3.08 | 0.00 | 31.54 |
| 1430111_PM_a_at | branched chain | Bcat1 | 12035 | −2.07 | 0.00 | 10.96 |
| | aminotransferase 1, | | | | | |
| | cytosolic | | | | | |
| 1430128_PM_a_at | receptor accessory protein 6 | Reep6 | 70335 | 1.51 | 0.00 | 5.50 |
| 1430135_PM_at | deoxyribonuclease II | Dnase2a | 13423 | 2.15 | 0.00 | 17.46 |
| | alpha | | | | | |
| 1431320_PM_a_at | myosin VA | Myo5a | 17918 | −2.35 | 0.00 | 15.82 |
| 1431905_PM_s_at | RIKEN cDNA | 4933427G17Rik | 74466 | 1.59 | 0.00 | 0.68 |
| | 4933427G17 gene | | | | | |
| 1433617_PM_s_at | UDP-Gal:betaGlcNAc | B4galt5 | 56336 | −1.51 | 0.00 | 18.34 |
| | beta 1,4- | | | | | |
| | galactosyltransferase, | | | | | |
| | polypeptide 5 | | | | | |
| 1433966_PM_x_at | asparagine synthetase | Asns | 27053 | 2.49 | 0.00 | 14.74 |
| 1434050_PM_at | vacuolar protein sorting 8 | Vps8 | 209018 | −1.84 | 0.00 | 5.70 |
| | homolog (S. cerevisiae) | | | | | |
| 1434716_PM_at | hepatitis A virus cellular | Havcr1 | 171283 | 4.79 | 0.00 | 12.58 |
| | receptor 1 | | | | | |
| 1434919_PM_at | alkylglycerone phosphate | Agps | 228061 | −1.80 | 0.00 | 17.61 |
| | synthase | | | | | |
| 1435646_PM_at | inhibitor of kappaB kinase | Ikbkg | 16151 | 1.76 | 0.00 | 11.72 |
| | gamma | | | | | |
| 1435663_PM_at | estrogen receptor 1 | Esr1 | 13982 | −1.75 | 0.00 | 11.80 |
| | (alpha) | | | | | |
| 1435828_PM_at | avian musculoaponeurotic | Maf | 17132 | 1.53 | 0.00 | 12.88 |
| | fibrosarcoma (v-maf) | | | | | |
| | AS42 oncogene homolog | | | | | |
| 1436051_PM_at | myosin VA | Myo5a | 17918 | −2.04 | 0.00 | 18.67 |
| 1436101_PM_at | ring finger protein 24 | Rnf24 | 51902 | −2.01 | 0.00 | 10.29 |
| 1436291_PM_a_at | dihydropyrimidinase | Dpys | 64705 | 1.51 | 0.00 | 2.46 |
| 1436771_PM_x_at | phosphogluconate | Pgd | 110208 | 2.03 | 0.00 | 33.09 |
| | dehydrogenase | | | | | |
| 1437380_PM_x_at | phosphogluconate | Pgd | 110208 | 2.05 | 0.00 | 32.56 |
| | dehydrogenase | | | | | |
| 1437467_PM_at | activated leukocyte cell | Alcam | 11658 | 1.93 | 0.00 | 25.48 |
| | adhesion molecule | | | | | |
| 1437675_PM_at | solute carrier family 8 | Slc8a1 | 20541 | −1.86 | 0.00 | 4.91 |
| | (sodium/calcium | | | | | |
| | exchanger), member 1 | | | | | |
| 1437870_PM_at | solute carrier organic | Slco4c1 | 227394 | −1.69 | 0.00 | 7.44 |
| | anion transporter family, | | | | | |
| | member 4C1 | | | | | |
| 1437932_PM_a_at | claudin 1 | Cldn1 | 12737 | 1.57 | 0.00 | 7.95 |
| 1438204_PM_at | Histone cluster 1, H1c | Hist1h1c | 50708 | 1.82 | 0.00 | 12.28 |
| 1438627_PM_x_at | phosphogluconate | Pgd | 110208 | 1.83 | 0.00 | 23.75 |
| | dehydrogenase | | | | | |
| 1438841_PM_s_at | arginase type II | Arg2 | 11847 | 2.63 | 0.00 | 12.59 |
| 1439695_PM_a_at | kinesin family member | Kif20b | 240641 | −2.85 | 0.00 | 4.40 |
| | 20B | | | | | |
| 1440008_PM_at | RIKEN cDNA | 2310043L19Rik | 75589 | 1.86 | 0.00 | 9.22 |
| | 2310043L19 gene | | | | | |
| 1440058_PM_at | solute carrier family 22 | Slc22a14 | 382113 | 1.54 | 0.00 | 7.12 |
| | (organic cation | | | | | |
| | transporter), member 14 | | | | | |
| 1440227_PM_at | solute carrier family 5 | Slc5a3 | 53881 | −1.50 | 0.00 | 0.67 |
| | (inositol transporters), | | | | | |
| | member 3 | | | | | |
| 1440330_PM_at | Histone cluster 1, H2be | Hist1h2be | 319179 | 2.92 | 0.00 | 10.15 |
| 1440688_PM_at | Rho GTPase activating | Arhgap26 | 71302 | −1.57 | 0.00 | 2.13 |
| | protein 26 | | | | | |
| 1440965_PM_at | phosphatidylinositol | Pigl | 327942 | 1.52 | 0.00 | 11.14 |
| | glycan anchor | | | | | |
| | biosynthesis, class L | | | | | |
| 1441333_PM_at | — | — | — | −2.46 | 0.00 | 9.13 |
| 1441944_PM_s_at | G protein-coupled | Gpr135 | 238252 | 1.59 | 0.00 | 11.39 |
| | receptor 135 | | | | | |
| 1441971_PM_at | — | — | — | 1.51 | 0.00 | 3.07 |
| 1441979_PM_at | cDNA sequence | BC060267 | 212516 | 1.74 | 0.00 | 11.09 |
| | BC060267 | | | | | |
| 1442145_PM_at | ATPase type 13A3 | Atp13a3 | 224088 | 1.62 | 0.00 | 18.14 |
| 1442291_PM_at | lysophosphatidic acid | Lpar2 | 53978 | 1.93 | 0.00 | 7.47 |
| | receptor 2 | | | | | |
| 1442588_PM_at | predicted gene 5101 | Gm5101 | 329217 | −1.50 | 0.00 | 12.56 |
| 1443870_PM_at | ATP-binding cassette, | Abcc4 | 239273 | 2.07 | 0.00 | 23.13 |
| | sub-family C | | | | | |
| | (CFTR/MRP), member 4 | | | | | |
| 1443964_PM_at | transmembrane inner ear | Tmie | 20776 | 1.65 | 0.00 | 9.16 |
| 1444242_PM_at | Solute carrier organic | Slco2a1 | 24059 | 1.63 | 0.00 | 6.87 |
| | anion transporter family, | | | | | |
| | member 2a1 | | | | | |
| 1444487_PM_at | lecithin-retinol | Lrat | 79235 | 1.72 | 0.00 | 6.60 |
| | acyltransferase | | | | | |
| | (phosphatidylcholine- | | | | | |
| | retinol-O-acyltransferase) | | | | | |
| 1444920_PM_at | — | — | — | −1.56 | 0.00 | 3.44 |
| 1445089_PM_at | DNA segment, Chr 16, | D16Ertd778e | 52714 | −2.25 | 0.00 | 7.78 |
| | ERATO Doi 778, | | | | | |
| | expressed | | | | | |
| 1445565_PM_at | Histone cluster 1, H2be | Hist1h2be | 319179 | 2.48 | 0.00 | 8.12 |
| 1446368_PM_at | RIKEN cDNA | 9130221J18Rik | 102123 | −1.53 | 0.00 | 7.22 |
| | 9130221J18 gene | | | | | |
| 1446742_PM_at | Nuclear factor I/A | Nfia | 18027 | −1.74 | 0.00 | 1.16 |
| 1447356_PM_at | cDNA sequence | AB099516 /// | 380975 /// | −1.57 | 0.00 | 14.78 |
| | AB099516 /// HIG1 | Higd1c | 554292 | | | |
| | domain family, member | | | | | |
| | 1C | | | | | |
| 1447396_PM_at | — | — | — | −1.52 | 0.00 | 0.94 |
| 1447849_PM_s_at | avian musculoaponeurotic | Maf | 17132 | 1.54 | 0.00 | 10.88 |
| | fibrosarcoma (v-maf) | | | | | |
| | AS42 oncogene homolog | | | | | |
| 1448160_PM_at | lymphocyte cytosolic | Lcp1 | 18826 | 1.54 | 0.00 | 2.49 |
| | protein 1 | | | | | |
| 1448330_PM_at | glutathione S-transferase, | Gstm1 | 14862 | 1.54 | 0.00 | 34.55 |
| | mu 1 | | | | | |
| 1448354_PM_at | glucose-6-phosphate | G6pdx | 14381 | 1.70 | 0.00 | 18.30 |
| | dehydrogenase X-linked | | | | | |
| 1448482_PM_at | solute carrier family 39 | Slc39a8 | 67547 | −1.55 | 0.00 | 0.13 |
| | (metal ion transporter), | | | | | |
| | member 8 | | | | | |
| 1448766_PM_at | gap junction protein, beta 1 | Gjb1 | 14618 | 1.81 | 0.00 | 16.12 |
| 1448767_PM_s_at | gap junction protein, beta 1 | Gjb1 | 14618 | 1.67 | 0.00 | 22.45 |
| 1448894_PM_at | aldo-keto reductase family | Akr1b8 | 14187 | 1.68 | 0.00 | 2.39 |
| | 1, member B8 | | | | | |
| 1449002_PM_at | pleckstrin homology-like | Phlda3 | 27280 | 1.81 | 0.00 | 6.43 |
| | domain, family A, | | | | | |
| | member 3 | | | | | |
| 1449065_PM_at | acyl-CoA thioesterase 1 | Acot1 | 26897 | 1.59 | 0.00 | 3.70 |
| 1449575_PM_a_at | glutathione S-transferase, | Gstp1 | 14870 | 1.54 | 0.00 | 28.62 |
| | pi 1 | | | | | |
| 1449610_PM_at | E1A binding protein p400 | Ep400 | 75560 | −1.67 | 0.00 | 1.48 |
| 1449937_PM_at | endonuclease, polyU- | Endou | 19011 | 1.89 | 0.00 | 10.54 |
| | specific | | | | | |
| 1450409_PM_a_at | solute carrier family 48 | Slc48a1 | 67739 | 1.53 | 0.00 | 27.05 |
| | (heme transporter), | | | | | |
| | member 1 | | | | | |
| 1450702_PM_at | hemochromatosis | Hfe | 15216 | 1.67 | 0.00 | 18.95 |
| 1450869_PM_at | fibroblast growth factor 1 | Fgf1 | 14164 | −1.58 | 0.00 | 12.58 |
| 1450871_PM_a_at | branched chain | Bcat1 | 12035 | −2.20 | 0.00 | 20.77 |
| | aminotransferase 1, | | | | | |
| | cytosolic | | | | | |
| 1451095_PM_at | asparagine synthetase | Asns | 27053 | 2.87 | 0.00 | 17.57 |
| 1451386_PM_at | biliverdin reductase B | Blvrb | 233016 | 2.23 | 0.00 | 29.04 |
| | (flavin reductase | | | | | |
| | (NADPH)) | | | | | |
| 1451607_PM_at | kallikrein 1-related | Klk1b21 | 16616 | 1.57 | 0.00 | 0.04 |
| | peptidase b21 | | | | | |
| 1451680_PM_at | sulfiredoxin 1 homolog | Srxn1 | 76650 | 2.09 | 0.00 | 7.66 |
| | (S. cerevisiae) | | | | | |
| 1452333_PM_at | SWI/SNF related, matrix | Smarca2 | 67155 | −1.60 | 0.00 | 13.26 |
| | associated, actin | | | | | |
| | dependent regulator of | | | | | |
| | chromatin, subfamily a, | | | | | |
| | member 2 | | | | | |
| 1452418_PM_at | RIKEN cDNA | 1200016E24Rik | 319202 | 1.50 | 0.00 | 5.97 |
| | 1200016E24 gene | | | | | |
| 1452733_PM_at | pantothenate kinase 2 | Pank2 | 74450 | −1.97 | 0.00 | 13.30 |
| 1452934_PM_at | transmembrane channel- | Tmc5 | 74424 | 1.91 | 0.00 | 2.78 |
| | like gene family 5 | | | | | |
| 1453234_PM_at | RIKEN cDNA | 1300002K09Rik | 74152 | 1.88 | 0.00 | 6.92 |
| | 1300002K09 gene | | | | | |
| 1454690_PM_at | inhibitor of kappaB kinase | Ikbkg | 16151 | 1.54 | 0.00 | 20.76 |
| | gamma | | | | | |
| 1454810_PM_s_at | vacuolar protein sorting 8 | Vps8 | 209018 | −1.55 | 0.00 | 12.78 |
| | homolog (S. cerevisiae) | | | | | |
| 1454865_PM_at | solute carrier family 9 | Slc9a8 | 77031 | −2.40 | 0.00 | 28.37 |
| | (sodium/hydrogen | | | | | |
| | exchanger), member 8 | | | | | |
| 1455282_PM_x_at | aminolevulinic acid | Alas1 | 11655 | 1.70 | 0.00 | 9.61 |
| | synthase 1 | | | | | |
| 1455454_PM_at | aldo-keto reductase family | Akr1c19 | 432720 | 2.00 | 0.00 | 15.08 |
| | 1, member C19 | | | | | |
| 1455699_PM_at | branched chain | Bcat1 | 12035 | −2.27 | 0.00 | 4.36 |
| | aminotransferase 1, | | | | | |
| | cytosolic | | | | | |
| 1455722_PM_at | — | — | — | −1.53 | 0.00 | 11.50 |
| 1455959_PM_s_at | glutamate-cysteine ligase, | Gclc | 14629 | 1.64 | 0.00 | 5.28 |
| | catalytic subunit | | | | | |
| 1456722_PM_at | chordin-like 1 | Chrdl1 | 83453 | −1.55 | 0.00 | 2.03 |
| 1457110_PM_at | pantothenate kinase 1 | Pank1 | 75735 | −1.73 | 0.00 | 6.29 |
| 1458599_PM_at | — | — | — | 1.57 | 0.00 | 8.57 |
| 1459091_PM_at | — | — | — | 2.38 | 0.00 | 0.93 |
| 1459274_PM_at | G protein-coupled | Gpr135 | 238252 | 1.65 | 0.00 | 6.93 |
| | receptor 135 | | | | | |
| 1459661_PM_at | doublecortin domain | Dcdc2a | 195208 | 1.69 | 0.00 | 5.18 |
| | containing 2a | | | | | |
| 1460196_PM_at | carbonyl reductase 1 | Cbr1 | 12408 | 2.10 | 0.00 | 28.66 |
| 1460591_PM_at | estrogen receptor 1 | Esr1 | 13982 | −1.54 | 0.00 | 12.13 |
| | (alpha) | | | | | |
| 1460616_PM_at | solute carrier organic | Slco4c1 | 227394 | −2.08 | 0.00 | 28.70 |
| | anion transporter family, | | | | | |
| | member 4C1 | | | | | |
| 1460629_PM_at | tripartite motif-containing | Trim16 | 94092 | 1.51 | 0.00 | 6.29 |
| | 16 | | | | | |

100DMF-vs-100MMF, 2 h

| | | | | | | |
|---|---|---|---|---|---|---|
| 1439640_PM_at | — | — | — | 1.54 | 0.00 | 0.11 |

100DMF-vs-100MMF, 7 h

| | | | | | | |
|---|---|---|---|---|---|---|
| 1426464_PM_at | nuclear receptor subfamily | Nr1d1 | 217166 | 1.56 | 0.00 | 1.10 |
| | 1, group D, member 1 | | | | | |

100DMF-vs-100MMF, 12 h

| | | | | | | |
|---|---|---|---|---|---|---|
| 1420187_PM_at | expressed sequence | C76628 | 97687 | 1.59 | 0.00 | 0.69 |
| | C76628 | | | | | |
| 1441333_PM_at | — | — | — | 1.91 | 0.00 | 2.00 |

LIVER
100DMF-vs-Veh, 2 h

| | | | | | | |
|---|---|---|---|---|---|---|
| 1417801_PM_a_at | PTPRF interacting protein, binding | Ppfibp2 | 19024 | 2.38 | 0.00 | 10.95 |
| | protein 2 (liprin beta 2) | | | | | |
| 1419627_PM_s_at | C-type lectin domain family 4, | Clec4n | 56620 | 2.34 | 0.00 | 3.78 |
| | member n | | | | | |
| 1419942_PM_at | Sulfiredoxin 1 homolog (S. cerevisiae) | Srxn1 | 76650 | 1.69 | 0.00 | 12.22 |
| 1420804_PM_s_at | C-type lectin domain family 4, | Clec4d | 17474 | 2.30 | 0.00 | 3.53 |
| | member d | | | | | |
| 1426875_PM_s_at | sulfiredoxin 1 homolog (S. cerevisiae) | Srxn1 | 76650 | 1.64 | 0.00 | 10.99 |
| 1435830_PM_a_at | RIKEN cDNA 5430435G22 gene | 5430435G22Rik | 226421 | 1.79 | 0.00 | 5.60 |
| 1436771_PM_x_at | phosphogluconate dehydrogenase | Pgd | 110208 | 1.59 | 0.00 | 9.52 |
| 1437380_PM_x_at | phosphogluconate dehydrogenase | Pgd | 110208 | 1.61 | 0.00 | 8.65 |
| 1438953_PM_at | c-fos induced growth factor | Figf | 14205 | 2.23 | 0.00 | 3.11 |
| 1438954_PM_x_at | c-fos induced growth factor | Figf | 14205 | 1.90 | 0.00 | 1.73 |
| 1443159_PM_at | RIKEN cDNA 9130221J17 gene | 9130221J17Rik | 319693 | 1.54 | 0.00 | 8.92 |
| 1444516_PM_at | — | — | — | 1.96 | 0.00 | 7.18 |
| 1447411_PM_at | — | — | — | 2.57 | 0.00 | 7.91 |
| 1448239_PM_at | heme oxygenase (decycling) 1 | Hmox1 | 15368 | 2.37 | 0.00 | 1.30 |
| 1448894_PM_at | aldo-keto reductase family 1, | Akr1b8 | 14187 | 2.18 | 0.00 | 12.42 |
| | member B8 | | | | | |
| 1449528_PM_at | c-fos induced growth factor | Figf | 14205 | 1.66 | 0.00 | 1.55 |
| 1451680_PM_at | sulfiredoxin 1 homolog (S. cerevisiae) | Srxn1 | 76650 | 1.59 | 0.00 | 6.27 |
| 1452233_PM_at | ATP-binding cassette, sub-family C | Abcc1 | 17250 | 1.75 | 0.00 | 3.19 |
| | (CFTR/MRP), member 1 | | | | | |

100DMF-vs-Veh, 7 h

| | | | | | | |
|---|---|---|---|---|---|---|
| 1419627_PM_s_at | C-type lectin domain family | Clec4n | 56620 | 2.01 | 0.00 | 1.11 |
| | 4, member n | | | | | |
| 1423627_PM_at | NAD(P)H dehydrogenase, | Nqo1 | 18104 | 1.81 | 0.00 | 0.98 |
| | quinone 1 | | | | | |
| 1435495_PM_at | adenosine A1 receptor | Adora1 | 11539 | 1.50 | 0.00 | 1.99 |
| 1448894_PM_at | aldo-keto reductase family | Akr1b8 | 14187 | 1.58 | 0.00 | 1.03 |
| | 1, member B8 | | | | | |
| 1450759_PM_at | bone morphogenetic protein 6 | Bmp6 | 12161 | 1.57 | 0.00 | 0.18 |

100DMF-vs-Veh, 12 h

| | | | | | | |
|---|---|---|---|---|---|---|
| 1424835_PM_at | glutathione S- | Gstm4 | 14865 | 1.54 | 0.00 | 2.01 |
| | transferase, mu 4 | | | | | |

100MMF-vs-Veh, 2 h

| | | | | | | |
|---|---|---|---|---|---|---|
| 1417801_PM_a_at | PTPRF interacting protein, binding | Ppfibp2 | 19024 | 2.55 | 0.00 | 14.17 |
| | protein 2 (liprin beta 2) | | | | | |
| 1419038_PM_a_at | casein kinase 2, alpha 1 polypeptide | Csnk2a1 | 12995 | −1.62 | 0.00 | 0.18 |
| 1419942_PM_at | Sulfiredoxin 1 homolog (S. cerevisiae) | Srxn1 | 76650 | 1.68 | 0.00 | 12.63 |
| 1420804_PM_s_at | C-type lectin domain family 4, | Clec4d | 17474 | 2.05 | 0.00 | 1.25 |
| | member d | | | | | |
| 1424296_PM_at | glutamate-cysteine ligase, catalytic | Gclc | 14629 | 1.72 | 0.00 | 0.73 |
| | subunit | | | | | |
| 1426875_PM_s_at | sulfiredoxin 1 homolog (S. cerevisiae) | Srxn1 | 76650 | 1.63 | 0.00 | 11.61 |
| 1436771_PM_x_at | phosphogluconate dehydrogenase | Pgd | 110208 | 1.61 | 0.00 | 10.89 |
| 1437380_PM_x_at | phosphogluconate dehydrogenase | Pgd | 110208 | 1.65 | 0.00 | 10.56 |
| 1437867_PM_at | — | — | — | 1.50 | 0.00 | 0.01 |
| 1438953_PM_at | c-fos induced growth factor | Figf | 14205 | 2.25 | 0.00 | 3.79 |
| 1438954_PM_x_at | c-fos induced growth factor | Figf | 14205 | 1.79 | 0.00 | 0.60 |
| 1440091_PM_at | Meis homeobox 2 | Meis2 | 17536 | −1.67 | 0.00 | 5.51 |
| 1443159_PM_at | RIKEN cDNA 9130221J17 gene | 9130221J17Rik | 319693 | 1.57 | 0.00 | 10.68 |
| 1444516_PM_at | — | — | — | 2.19 | 0.00 | 12.38 |
| 1447411_PM_at | — | — | — | 2.81 | 0.00 | 11.17 |
| 1448239_PM_at | heme oxygenase (decycling) 1 | Hmox1 | 15368 | 2.41 | 0.00 | 2.09 |
| 1448348_PM_at | cell cycle associated protein 1 | Caprin1 | 53872 | −1.56 | 0.00 | 2.99 |
| 1448894_PM_at | aldo-keto reductase family 1, member | Akr1b8 | 14187 | 2.27 | 0.00 | 14.85 |
| | B8 | | | | | |
| 1451680_PM_at | sulfiredoxin 1 homolog (S. cerevisiae) | Srxn1 | 76650 | 1.55 | 0.00 | 5.56 |
| 1452233_PM_at | ATP-binding cassette, sub-family C | Abcc1 | 17250 | 1.86 | 0.00 | 5.89 |
| | (CFTR/MRP), member 1 | | | | | |
| 1452247_PM_at | fragile X mental retardation gene 1, | Fxr1 | 14359 | −1.53 | 0.00 | 1.30 |
| | autosomal homolog | | | | | |
| 1455016_PM_at | PRP38 pre-mRNA processing factor | Prpf38b | 66921 | −1.53 | 0.00 | 6.51 |
| | 38 (yeast) domain containing B | | | | | |
| 1455959_PM_s_at | glutamate-cysteine ligase, catalytic | Gclc | 14629 | 1.64 | 0.00 | 2.88 |
| | subunit | | | | | |

100MMF-vs-Veh, 7 h

| | | | | | | |
|---|---|---|---|---|---|---|
| 1419627_PM_s_at | C-type lectin domain family 4, | Clec4n | 56620 | 2.40 | 0.00 | 5.48 |
| | member n | | | | | |
| 1419942_PM_at | Sulfiredoxin 1 homolog (S. cerevisiae) | Srxn1 | 76650 | 1.52 | 0.00 | 7.47 |
| 1423627_PM_at | NAD(P)H dehydrogenase, | Nqo1 | 18104 | 2.20 | 0.00 | 6.83 |
| | quinone 1 | | | | | |
| 1424626_PM_at | RIKEN cDNA 2010003K11 | 2010003K11Rik | 69861 | 2.20 | 0.00 | 1.48 |
| | gene | | | | | |
| 1426875_PM_s_at | sulfiredoxin 1 homolog (S. cerevisiae) | Srxn1 | 76650 | 1.53 | 0.00 | 8.21 |
| 1427357_PM_at | cytidine deaminase | Cda | 72269 | 1.92 | 0.00 | 1.59 |
| 1429001_PM_at | pirin | Pir | 69656 | 1.50 | 0.00 | 3.62 |
| 1430175_PM_at | RIKEN cDNA 4930588G05 | 4930588G05Rik | 78817 | −1.76 | 0.00 | 0.68 |
| | gene | | | | | |
| 1434582_PM_at | ELKS/RAB6-interacting/CAST | Erc2 | 238988 | −1.95 | 0.00 | 5.77 |
| | family member 2 | | | | | |
| 1445815_PM_at | frizzled homolog 8 | Fzd8 | 14370 | −1.81 | 0.00 | 0.64 |
| | (Drosophila) | | | | | |
| 1448354_PM_at | glucose-6-phosphate | G6pdx | 14381 | 1.61 | 0.00 | 0.74 |
| | dehydrogenase X-linked | | | | | |
| 1448894_PM_at | aldo-keto reductase family 1, | Akr1b8 | 14187 | 1.75 | 0.00 | 4.85 |
| | member B8 | | | | | |
| 1449498_PM_at | macrophage receptor with | Marco | 17167 | 2.44 | 0.00 | 0.39 |
| | collagenous structure | | | | | |
| 1450759_PM_at | bone morphogenetic protein 6 | Bmp6 | 12161 | 1.65 | 0.00 | 1.89 |
| 1451680_PM_at | sulfiredoxin 1 homolog (S. cerevisiae) | Srxn1 | 76650 | 1.58 | 0.00 | 7.51 |

100MMF-vs-Veh, 12 h

| | | | | | | |
|---|---|---|---|---|---|---|
| 1416235_PM_at | leucine rich repeat containing 59 | Lrrc59 | 98238 | 1.65 | 0.00 | 3.69 |
| 1417441_PM_at | DnaJ (Hsp40) homolog, subfamily C, | Dnajc12 | 30045 | 2.53 | 0.00 | 1.11 |
| | member 12 | | | | | |
| 1423290_PM_at | hypoxia up-regulated 1 | Hyou1 | 12282 | 1.82 | 0.00 | 0.58 |
| 1423291_PM_s_at | hypoxia up-regulated 1 | Hyou1 | 12282 | 1.52 | 0.00 | 2.86 |
| 1425921_PM_a_at | RIKEN cDNA 1810055G02 gene | 1810055G02Rik | 72056 | 1.86 | 0.00 | 2.21 |
| 1428154_PM_s_at | phosphatidic acid phosphatase type 2 | Ppapdc1b | 71910 | 1.87 | 0.00 | 3.68 |
| | domain containing 1B | | | | | |
| 1431214_PM_at | predicted gene 3579 | Gm3579 | 100041932 | 2.74 | 0.00 | 2.23 |
| 1431334_PM_a_at | RIKEN cDNA 4933433P14 gene | 4933433P14Rik | 66787 | 1.81 | 0.00 | 2.33 |
| 1433833_PM_at | fibronectin type III domain containing 3B | Fndc3b | 72007 | 1.66 | 0.00 | 2.57 |
| 1439117_PM_at | calmin | Clmn | 94040 | −1.54 | 0.00 | 0.80 |
| 1441988_PM_at | protein phosphatase 1K (PP2C domain | Ppm1k | 243382 | −1.56 | 0.00 | 1.95 |
| | containing) | | | | | |
| 1448471_PM_a_at | cytotoxic T lymphocyte-associated | Ctla2a | 13024 | 1.63 | 0.00 | 0.43 |
| | protein 2 alpha | | | | | |
| 1448574_PM_at | non-metastatic cells 6, protein expressed | Nme6 | 54369 | 1.57 | 0.00 | 0.88 |
| | in (nucleoside-diphosphate kinase) | | | | | |
| 1450971_PM_at | growth arrest and DNA-damage- | Gadd45b | 17873 | 1.50 | 0.00 | 0.15 |
| | inducible 45 beta | | | | | |
| 1453103_PM_at | actin-binding LIM protein 1 | Ablim1 | 226251 | −1.51 | 0.00 | 0.34 |
| 1460256_PM_at | carbonic anhydrase 3 | Car3 | 12350 | −1.57 | 0.00 | 1.82 |

100DMF-vs-100MMF, 2 h
None
100DMF-vs-100MMF, 7 h

| | | | | | | |
|---|---|---|---|---|---|---|
| 1448147_PM_at | tumor necrosis factor receptor | Tnfrsf19 | 29820 | 1.73 | 0.00 | 0.24 |
| | superfamily, member 19 | | | | | |

100DMF-vs-100MMF, 12 h

| | | | | | | |
|---|---|---|---|---|---|---|
| 1415977_PM_at | myo-inositol 1-phosphate | Isyna1 | 71780 | −1.76 | 0.00 | 0.23 |
| | synthase A1 | | | | | |
| 1417441_PM_at | DnaJ (Hsp40) homolog, | Dnajc12 | 30045 | −2.49 | 0.00 | 0.82 |
| | subfamily C, member 12 | | | | | |
| 1417507_PM_at | cytochrome b-561 | Cyb561 | 13056 | −2.02 | 0.00 | 1.31 |
| 1419005_PM_at | crystallin, beta B3 | Crybb3 | 12962 | −1.69 | 0.00 | 1.95 |
| 1446368_PM_at | RIKEN cDNA 9130221J18 gene | 9130221J18Rik | 102123 | −1.93 | 0.00 | 0.24 |

SPLEEN
100DMF-vs-Veh, 2 h

| | | | | | | |
|---|---|---|---|---|---|---|
| 1416497_PM_at | protein disulfide isomerase associated 4 | Pdia4 | 12304 | −1.60 | 0.00 | 3.53 |
| 1419435_PM_at | aldehyde oxidase 1 | Aox1 | 11761 | 2.61 | 0.00 | 4.66 |
| 1419942_PM_at | Sulfiredoxin 1 homolog (S. cerevisiae) | Srxn1 | 76650 | 2.16 | 0.00 | 7.81 |
| 1421529_PM_a_at | thioredoxin reductase 1 | Txnrd1 | 50493 | 1.55 | 0.00 | 25.62 |
| 1424022_PM_at | oxidative stress induced growth inhibitor 1 | Osgin1 | 71839 | 1.78 | 0.00 | 0.31 |
| 1424296_PM_at | glutamate-cysteine ligase, catalytic subunit | Gclc | 14629 | 1.76 | 0.00 | 0.33 |
| 1424486_PM_a_at | thioredoxin reductase 1 | Txnrd1 | 50493 | 2.41 | 0.00 | 7.23 |
| 1424487_PM_x_at | thioredoxin reductase 1 | Txnrd1 | 50493 | 1.73 | 0.00 | 7.08 |
| 1426875_PM_s_at | sulfiredoxin 1 homolog (S. cerevisiae) | Srxn1 | 76650 | 2.02 | 0.00 | 9.98 |
| 1434797_PM_at | kin of IRRE like (Drosophila) | Kirrel | 170643 | −1.54 | 0.00 | 0.16 |
| 1443159_PM_at | RIKEN cDNA 9130221J17 gene | 9130221J17Rik | 319693 | 1.52 | 0.00 | 10.78 |
| 1448916_PM_at | v-maf musculoaponeurotic fibrosarcoma | Mafg | 17134 | 1.58 | 0.00 | 1.11 |
| | oncogene family, protein G (avian) | | | | | |
| 1451680_PM_at | sulfiredoxin 1 homolog (S. cerevisiae) | Srxn1 | 76650 | 1.53 | 0.00 | 3.65 |

100DMF-vs-Veh, 7 h

| | | | | | | |
|---|---|---|---|---|---|---|
| 1419942_PM_at | Sulfiredoxin 1 homolog (S. cerevisiae) | Srxn1 | 76650 | 1.67 | 0.00 | 0.88 |
| 1426875_PM_s_at | sulfiredoxin 1 homolog (S. cerevisiae) | Srxn1 | 76650 | 1.50 | 0.00 | 0.25 |

100DMF-vs-Veh, 12 h
None
100MMF-vs-Veh, 2 h

| | | | | | | |
|---|---|---|---|---|---|---|
| 1416497_PM_at | protein disulfide isomerase | Pdia4 | 12304 | −1.59 | 0.00 | 2.87 |
| | associated 4 | | | | | |
| 1419435_PM_at | aldehyde oxidase 1 | Aox1 | 11761 | 2.35 | 0.00 | 2.09 |
| 1419942_PM_at | Sulfiredoxin 1 homolog (S. cerevisiae) | Srxn1 | 76650 | 2.02 | 0.00 | 4.97 |
| 1421529_PM_a_at | thioredoxin reductase 1 | Txnrd1 | 50493 | 1.53 | 0.00 | 22.99 |
| 1424022_PM_at | oxidative stress induced | Osgin1 | 71839 | 1.83 | 0.00 | 0.58 |
| | growth inhibitor 1 | | | | | |
| 1424486_PM_a_at | thioredoxin reductase 1 | Txnrd1 | 50493 | 2.14 | 0.00 | 3.49 |
| 1426875_PM_s_at | sulfiredoxin 1 homolog (S. cerevisiae) | Srxn1 | 76650 | 1.92 | 0.00 | 7.31 |
| 1426937_PM_at | RIKEN cDNA 6330406I15 | 6330406I15Rik | 70717 | −1.58 | 0.00 | 1.13 |
| | gene | | | | | |
| 1434797_PM_at | kin of IRRE like (Drosophila) | Kirrel | 170643 | −1.65 | 0.00 | 1.80 |
| 1434951_PM_at | armadillo repeat containing 8 | Armc8 | 74125 | −1.54 | 0.00 | 4.23 |
| 1443159_PM_at | RIKEN cDNA 9130221J17 | 9130221J17Rik | 319693 | 1.55 | 0.00 | 11.29 |
| | gene | | | | | |
| 1446490_PM_at | — | — | — | −1.76 | 0.00 | 2.19 |

100MMF-vs-Veh, 7 h

| | | | | | | |
|---|---|---|---|---|---|---|
| 1419942_PM_at | Sulfiredoxin 1 homolog (S. cerevisiae) | Srxn1 | 76650 | 1.87 | 0.00 | 4.29 |
| 1426875_PM_s_at | sulfiredoxin 1 homolog (S. cerevisiae) | Srxn1 | 76650 | 1.69 | 0.00 | 4.34 |

100MMF-vs-Veh, 12 h
None
100DMF-vs-100MMF, 2 h
None
100DMF-vs-100MMF, 7 h
None
100DMF-vs-100MMF, 12 h
None

### APPENDIX C

### Naïve Multidose Gene Lists

| | | | | | |
|---|---|---|---|---|---|
| | Gene | Entrez | | p. | |
| Gene Title | Symbol | Gene | FC | value | lods |

WHOLE BLOOD
100DMF-vs-Veh

| | | | | | | |
|---|---|---|---|---|---|---|
| 1416316_PM_at | solute carrier family 27 (fatty acid transporter), | Slc27a2 | 26458 | −2.37 | 0.00 | 3.98 |
| | member 2 | | | | | |
| 1424214_PM_at | prostate androgen-regulated mucin-like protein 1 | Parm1 | 231440 | −1.68 | 0.00 | 1.50 |
| 1428284_PM_at | RIKEN cDNA 8430427H17 gene | 8430427H17Rik | 329540 | −1.73 | 0.00 | 0.79 |
| 1430700_PM_a_at | phospholipase A2, group VII (platelet- | Pla2g7 | 27226 | 1.65 | 0.00 | 0.04 |
| | activating factor acetylhydrolase, plasma) | | | | | |
| 1439574_PM_at | RIKEN cDNA 1110020A21 gene | 1110020A21Rik | 68531 | −1.77 | 0.00 | 0.56 |

100MMF-vs-Veh

| | | | | | | |
|---|---|---|---|---|---|---|
| 1415904_PM_at | lipoprotein lipase | Lpl | 16956 | 2.65 | 0.00 | 1.86 |
| 1416273_PM_at | tumor necrosis factor, alpha-induced protein 2 | Tnfaip2 | 21928 | 1.81 | 0.00 | 2.00 |
| 1416316_PM_at | solute carrier family 27 (fatty acid transporter), | Slc27a2 | 26458 | −1.93 | 0.00 | 0.32 |
| | member 2 | | | | | |
| 1416416_PM_x_at | glutathione S-transferase, mu 1 | Gstm1 | 14862 | 1.59 | 0.00 | 2.99 |
| 1425546_PM_a_at | transferrin | Trf | 22041 | 1.56 | 0.00 | 1.53 |
| 1430700_PM_a_at | phospholipase A2, group VII (platelet- | Pla2g7 | 27226 | 1.76 | 0.00 | 1.56 |
| | activating factor acetylhydrolase, plasma) | | | | | |
| 1436329_PM_at | early growth response 3 | Egr3 | 13655 | −1.60 | 0.00 | 0.38 |
| 1438855_PM_x_at | tumor necrosis factor, alpha-induced protein 2 | Tnfaip2 | 21928 | 1.52 | 0.00 | 1.48 |
| 1447339_PM_at | — | — | — | 2.49 | 0.00 | 3.05 |
| 1460220_PM_a_at | colony stimulating factor 1 (macrophage) | Csf1 | 12977 | 1.75 | 0.00 | 1.95 |
| 1460318_PM_at | cysteine and glycine-rich protein 3 | Csrp3 | 13009 | 1.52 | 0.00 | 0.18 |

100DMF-vs-100MMF

None

BRAIN_HEMI
100DMF-vs-Veh

| | | | | | | |
|---|---|---|---|---|---|---|
| 1433201_PM_at | RIKEN cDNA 2310079F09 gene | 2310079F09Rik | 70290 | 2.00 | 0.00 | 2.24 |
| 1433836_PM_a_at | RIKEN cDNA 8430408G22 gene | 8430408G22Rik | 213393 | 1.77 | 0.00 | 7.48 |
| 1457257_PM_x_at | poliovirus receptor-related 3 | Pvrl3 | 58998 | −1.69 | 0.00 | 0.82 |

100MMF-vs-Veh

| | | | | | | |
|---|---|---|---|---|---|---|
| 1415908_PM_at | testis-specific protein, Y-encoded-like 1 | Tspyl1 | 22110 | −1.68 | 0.00 | 7.12 |
| 1416039_PM_x_at | cysteine rich protein 61 | Cyr61 | 16007 | 2.31 | 0.00 | 0.72 |
| 1416065_PM_a_at | ankyrin repeat domain 10 | Ankrd10 | 102334 | −1.58 | 0.00 | 8.61 |
| 1416290_PM_a_at | proteasome (prosome, macropain) 26S | Psmc4 | 23996 | −1.64 | 0.00 | 7.80 |
| | subunit, ATPase, 4 | | | | | |
| 1416422_PM_a_at | Sjogren syndrome antigen B | Ssb | 20823 | −1.69 | 0.00 | 8.20 |
| 1416476_PM_a_at | ubiquitin-conjugating enzyme E2D2 | Ube2d2 | 56550 | −1.66 | 0.00 | 2.56 |
| 1416497_PM_at | protein disulfide isomerase associated 4 | Pdia4 | 12304 | 1.60 | 0.00 | 0.25 |
| 1416499_PM_a_at | dynactin 6 | Dctn6 | 22428 | −1.95 | 0.00 | 15.72 |
| 1416514_PM_a_at | fascin homolog 1, actin bundling protein | Fscn1 | 14086 | −1.54 | 0.00 | 6.60 |
| | (Strongylocentrotus purpuratus) | | | | | |
| 1416711_PM_at | T-box brain gene 1 | Tbr1 | 21375 | −1.58 | 0.00 | 2.70 |
| 1416814_PM_at | cytotoxic granule-associated RNA | Tia1 | 21841 | −1.54 | 0.00 | 5.12 |
| | binding protein 1 | | | | | |
| 1417188_PM_s_at | ubiquitin-conjugating enzyme E2K | Ube2k | 53323 | −1.71 | 0.00 | 9.68 |
| | (UBC1 homolog, yeast) | | | | | |
| 1417517_PM_at | pleiomorphic adenoma gene-like 2 | Plagl2 | 54711 | 1.54 | 0.00 | 1.56 |
| 1417602_PM_at | period homolog 2 (Drosophila) | Per2 | 18627 | −1.53 | 0.00 | 4.12 |
| 1417770_PM_s_at | proteasome (prosome, macropain) | Psmc6 | 67089 | −1.81 | 0.00 | 15.59 |
| | 26S subunit, ATPase, 6 | | | | | |
| 1417815_PM_a_at | serine incorporator 3 | Serinc3 | 26943 | −1.66 | 0.00 | 11.80 |
| 1417980_PM_a_at | insulin induced gene 2 | Insig2 | 72999 | −1.59 | 0.00 | 6.52 |
| 1418047_PM_at | neurogenic differentiation 6 | Neurod6 | 11922 | −1.50 | 0.00 | 7.15 |
| 1418157_PM_at | similar to COUP-TFI /// nuclear receptor | LOC100046044 /// | 100046044 /// | −1.55 | 0.00 | 9.27 |
| | subfamily 2, group F, member 1 | Nr2f1 | 13865 | | | |
| 1418526_PM_at | splicing factor, arginine/serine-rich 13A | Sfrs13a | 14105 | −1.62 | 0.00 | 6.81 |
| 1418585_PM_at | cyclin H | Ccnh | 66671 | −1.69 | 0.00 | 4.40 |
| 1418690_PM_at | protein tyrosine phosphatase, receptor | Ptprz1 | 19283 | −1.56 | 0.00 | 7.58 |
| | type Z, polypeptide 1 | | | | | |
| 1419081_PM_at | autophagy-related 10 (yeast) | Atg10 | 66795 | −1.71 | 0.00 | 12.76 |
| 1419381_PM_at | telomeric repeat binding factor 2, | Terf2ip | 57321 | −1.78 | 0.00 | 8.09 |
| | interacting protein | | | | | |
| 1419565_PM_a_at | zinc finger protein X-linked | Zfx | 22764 | −1.75 | 0.00 | 9.50 |
| 1419801_PM_x_at | — | — | — | −1.59 | 0.00 | 1.11 |
| 1420042_PM_at | THO complex 1 | Thoc1 | 225160 | −1.75 | 0.00 | 7.70 |
| 1420053_PM_at | Proteasome (prosome, macropain) subunit, | Psmb1 | 19170 | −1.57 | 0.00 | 7.58 |
| | beta type 1 | | | | | |
| 1421889_PM_a_at | amyloid beta (A4) precursor-like protein 2 | Aplp2 | 11804 | 1.50 | 0.00 | 7.18 |
| 1421923_PM_at | SH3-domain binding protein 5 | Sh3bp5 | 24056 | −1.50 | 0.00 | 0.18 |
| | (BTK-associated) | | | | | |
| 1422329_PM_a_at | similar to neurotrophic tyrosine kinase, | LOC100047606 /// | 100047606 /// | 1.68 | 0.00 | 1.25 |
| | receptor, type 3 /// neurotrophic tyrosine | Ntrk3 | 18213 | | | |
| | kinase, receptor, type 3 | | | | | |
| 1422542_PM_at | G protein-coupled receptor 34 | Gpr34 | 23890 | −1.71 | 0.00 | 8.75 |
| 1422573_PM_at | adenosine monophosphate deaminase 3 | Ampd3 | 11717 | −1.57 | 0.00 | 7.52 |
| 1422716_PM_a_at | acid phosphatase 1, soluble | Acp1 | 11431 | −1.52 | 0.00 | 8.85 |
| 1423487_PM_at | cysteine-rich PDZ-binding protein | Cript | 56724 | −1.52 | 0.00 | 0.84 |
| 1423804_PM_a_at | isopentenyl-diphosphate delta isomerase | Idi1 | 319554 | −1.70 | 0.00 | 2.31 |
| 1424672_PM_at | Dmx-like 1 | Dmxl1 | 240283 | −1.51 | 0.00 | 10.12 |
| 1425350_PM_a_at | myelin basic protein expression factor 2, | Myef2 | 17876 | −1.50 | 0.00 | 5.01 |
| | represser | | | | | |
| 1425485_PM_at | myotubularin related protein 6 | Mtmr6 | 219135 | −1.51 | 0.00 | 4.20 |
| 1425495_PM_at | zinc finger protein 62 | Zfp62 | 22720 | −1.90 | 0.00 | 6.74 |
| 1426448_PM_at | praja1, RING-H2 motif containing | Pja1 | 18744 | −1.61 | 0.00 | 1.83 |
| 1426462_PM_at | gephyrin | Gphn | 268566 | −1.51 | 0.00 | 15.14 |
| 1426739_PM_at | downstream neighbor of SON | Donson | 60364 | −1.50 | 0.00 | 6.58 |
| 1427054_PM_s_at | ABI gene family, member 3 (NESH) | Abi3bp | 320712 | −2.12 | 0.00 | 1.45 |
| | binding protein | | | | | |
| 1427122_PM_at | coatomer protein complex, subunit | Copg2as2 | 100044236 | −1.72 | 0.00 | 5.60 |
| | gamma 2, antisense 2 | | | | | |
| 1427233_PM_at | teashirt zinc finger family member 1 | Tshz1 | 110796 | −1.83 | 0.00 | 3.70 |
| 1427269_PM_at | splicing factor, arginine/serine-rich 11 | Sfrs11 | 69207 | −1.60 | 0.00 | 4.61 |
| 1427467_PM_a_at | retinitis pigmentosa GTPase regulator | Rpgr | 19893 | −1.60 | 0.00 | 7.50 |
| 1427519_PM_at | adenosine A2a receptor | Adora2a | 11540 | 1.56 | 0.00 | 3.52 |
| 1427590_PM_at | zinc finger protein 39 | Zfp39 | 22698 | −1.60 | 0.00 | 5.60 |
| 1428091_PM_at | kelch-like 7 (Drosophila) | Klhl7 | 52323 | −1.75 | 0.00 | 8.02 |
| 1428162_PM_at | RIKEN cDNA 4933421E11 gene | 4933421E11Rik | 321000 | −1.50 | 0.00 | 3.96 |
| 1428210_PM_s_at | conserved helix-loop-helix ubiquitous kinase | Chuk | 12675 | −1.68 | 0.00 | 3.65 |
| 1428224_PM_at | heterogeneous nuclear ribonucleoprotein | Hnrpdl | 50926 | −1.60 | 0.00 | 3.97 |
| | D-like | | | | | |
| 1428333_PM_at | RIKEN cDNA 2900062L11 gene | 2900062L11Rik | 76976 | −1.50 | 0.00 | 7.00 |
| 1428369_PM_s_at | Rho GTPase activating protein 21 | Arhgap21 | 71435 | −1.66 | 0.00 | 11.24 |
| 1428586_PM_at | transmembrane protein 41B | Tmem41b | 233724 | −1.89 | 0.00 | 3.86 |
| 1428693_PM_at | RIKEN cDNA 2610044O15 gene | 2610044O15Rik | 72139 | −1.52 | 0.00 | 5.47 |
| 1429211_PM_at | cell adhesion molecule 2 | Cadm2 | 239857 | −1.76 | 0.00 | 11.98 |
| 1429216_PM_at | progestin and adipoQ receptor family member III | Paqr3 | 231474 | −1.56 | 0.00 | 5.69 |
| 1429371_PM_at | zinc finger protein 788 | Zfp788 | 67607 | −1.65 | 0.00 | 3.39 |
| 1429490_PM_at | Rap1 interacting factor 1 homolog (yeast) | Rif1 | 51869 | −1.82 | 0.00 | 6.17 |
| 1429581_PM_at | acyl-Coenzyme A dehydrogenase | Acad9 | 229211 | −1.55 | 0.00 | 4.04 |
| | family, member 9 | | | | | |
| 1429599_PM_a_at | methylenetetrahydrofolate dehydrogenase | Mthfd2l | 665563 | −1.71 | 0.00 | 4.94 |
| | (NADP+ dependent) 2-like | | | | | |
| 1429693_PM_at | disabled homolog 2 (Drosophila) | Dab2 | 13132 | 1.53 | 0.00 | 0.04 |
| 1429712_PM_at | predicted gene 14288 | Gm14288 | 13999 | −1.68 | 0.00 | 3.51 |
| 1429879_PM_at | RIKEN cDNA 0610037L13 gene | 0610037L13Rik | 74098 | −1.51 | 0.00 | 4.76 |
| 1430123_PM_a_at | aldo-keto reductase family 1, | Akr1a4 | 58810 | −1.75 | 0.00 | 17.69 |
| | member A4 (aldehyde reductase) | | | | | |
| 1433201_PM_at | RIKEN cDNA 2310079F09 gene | 2310079F09Rik | 70290 | 2.17 | 0.00 | 3.32 |
| 1433267_PM_at | inhibitor of growth family, member 1 | Ing1 | 26356 | 1.53 | 0.00 | 0.74 |
| 1433488_PM_x_at | glucosamine (N-acetyl)-6-sulfatase | Gns | 75612 | −2.01 | 0.00 | 4.66 |
| 1433659_PM_at | tubulin, gamma complex associated protein 4 | Tubgcp4 | 51885 | −1.54 | 0.00 | 8.45 |
| 1433823_PM_at | protein tyrosine phosphatase domain | Ptpdc1 | 218232 | −1.59 | 0.00 | 8.61 |
| | containing 1 | | | | | |
| 1433856_PM_at | diphosphoinositol pentakisphosphate kinase 2 | Ppip5k2 | 227399 | −1.57 | 0.00 | 2.92 |
| 1433897_PM_at | expressed sequence AI597468 | AI597468 | 103266 | −1.51 | 0.00 | 9.32 |
| 1433906_PM_at | clavesin 1 | Clvs1 | 74438 | −1.54 | 0.00 | 8.46 |
| 1433914_PM_at | expressed sequence AI747699 | AI747699 | 381236 | −2.28 | 0.00 | 8.55 |
| 1434056_PM_a_at | NADH dehydrogenase (ubiquinone) 1 beta | Ndufb6 | 230075 | −1.56 | 0.00 | 11.61 |
| | subcomplex, 6 | | | | | |
| 1434108_PM_at | F-box protein 11 | Fbxo11 | 225055 | −1.50 | 0.00 | 2.64 |
| 1434150_PM_a_at | HIG1 domain family, member 1C /// | Higd1c /// | 380975 /// | −1.60 | 0.00 | 6.35 |
| | methyltransferase like 7A1 /// | Mettl7a1 /// | 393082 /// | | | |
| | methyltransferase like 7A2 | Mettl7a2 | 70152 | | | |
| 1434236_PM_at | zinc finger, DHHC domain containing 20 | Zdhhc20 | 75965 | −1.64 | 0.00 | 3.63 |
| 1434249_PM_s_at | tripartite motif-containing 9 | Trim9 | 94090 | −1.58 | 0.00 | 4.18 |
| 1434425_PM_at | trichohyalin | Tchh | 99681 | −1.64 | 0.00 | 5.01 |
| 1434427_PM_a_at | ring finger protein 157 | Rnf157 | 217340 | −1.67 | 0.00 | 13.72 |
| 1434625_PM_at | RIKEN cDNA 4930432O21 gene | 4930432O21Rik | 74670 | −1.66 | 0.00 | 3.56 |
| 1434866_PM_x_at | carnitine palmitoyltransferase 1a, liver | Cpt1a | 12894 | −1.81 | 0.00 | 4.26 |
| 1434940_PM_x_at | regulator of G-protein signaling 19 | Rgs19 | 56470 | −1.51 | 0.00 | 6.33 |
| 1435146_PM_s_at | cell adhesion molecule 2 | Cadm2 | 239857 | −2.25 | 0.00 | 13.62 |
| 1435162_PM_at | protein kinase, cGMP-dependent, type II | Prkg2 | 19092 | −1.69 | 0.00 | 5.27 |
| 1435164_PM_s_at | ubiquitin-like modifier activating enzyme 3 | Uba3 | 22200 | −1.65 | 0.00 | 8.57 |
| 1435233_PM_at | nuclear receptor coactivator 2 | Ncoa2 | 17978 | −1.55 | 0.00 | 5.24 |
| 1435435_PM_at | cortactin binding protein 2 | Cttnbp2 | 30785 | −1.75 | 0.00 | 4.28 |
| 1436116_PM_x_at | adaptor protein, phosphotyrosine interaction, | Appl1 | 72993 | −1.62 | 0.00 | 1.11 |
| | PH domain and leucine zipper containing 1 | | | | | |
| 1436152_PM_a_at | hepatitis B virus x interacting protein | Hbxip | 68576 | −1.65 | 0.00 | 7.75 |
| 1436223_PM_at | integrin beta 8 | Itgb8 | 320910 | −1.59 | 0.00 | 2.21 |
| 1436298_PM_x_at | phosphoribosylaminoimidazole carboxylase, | Paics | 67054 | −1.57 | 0.00 | 10.52 |
| | phosphoribosylaminoribosylamino | | | | | |
| | imidazole, succinocarboxamide synthetase | | | | | |
| 1436390_PM_a_at | chloride channel CLIC-like 1 | Clcc1 | 229725 | −1.64 | 0.00 | 8.92 |
| 1436411_PM_at | ATPase type 13A5 | Atp13a5 | 268878 | −1.51 | 0.00 | 3.00 |
| 1436420_PM_a_at | importin 4 | Ipo4 | 75751 | −1.64 | 0.00 | 5.58 |
| 1436495_PM_s_at | zinc finger protein 260 | Zfp260 | 26466 | −1.65 | 0.00 | 6.89 |
| 1436600_PM_at | TOX high mobility group box family member 3 | Tox3 | 244579 | −1.56 | 0.00 | 6.70 |
| 1436646_PM_at | — | — | — | −1.53 | 0.00 | 7.61 |
| 1436689_PM_a_at | aldehyde dehydrogenase 9, subfamily A1 | Aldh9a1 | 56752 | −1.63 | 0.00 | 0.70 |
| 1436718_PM_at | neurexophilin 1 | Nxph1 | 18231 | −1.56 | 0.00 | 6.08 |
| 1436761_PM_s_at | family with sequence similarity 13, member C | Fam13c | 71721 | −1.83 | 0.00 | 10.96 |
| 1436772_PM_at | Glutamate receptor, ionotropic, AMPA4 | Gria4 | 14802 | −1.57 | 0.00 | 4.59 |
| | (alpha 4) | | | | | |
| 1436848_PM_x_at | inositol (myo)-1(or 4)-monophosphatase 1 | Impa1 | 55980 | −1.55 | 0.00 | 9.27 |
| 1436885_PM_a_at | calcium homeostasis endoplasmic reticulum protein | Cherp | 27967 | −1.99 | 0.00 | 6.84 |
| 1436915_PM_x_at | lysosomal-associated protein transmembrane 4B | Laptm4b | 114128 | −1.68 | 0.00 | 10.96 |
| 1436944_PM_x_at | phosphatidylserine decarboxylase, pseudogene | Pisd-ps1 /// | 236604 /// | −2.43 | 0.00 | 3.03 |
| | 1 /// phosphatidylserine decarboxylase, | Pisd-ps3 | 66776 | | | |
| | pseudogene 3 | | | | | |
| 1436946_PM_s_at | predicted gene 13342 /// predicted gene | Gm13342 /// | 100041120 /// | −1.70 | 0.00 | 14.88 |
| | 15776 /// predicted gene 3150 /// guanine | Gm15776 /// | 100041703 /// | | | |
| | nucleotide binding protein (G protein), | Gm3150 /// | 100043507 /// | | | |
| | gamma 5 /// similar to G protein | Gng5 /// | 100044719 /// | | | |
| | gamma-5 subunit | LOC100044719 | 14707 | | | |
| 1436959_PM_x_at | nasal embryonic LHRH factor | Nelf | 56876 | −1.65 | 0.00 | 12.57 |
| 1436989_PM_s_at | solute carrier family 12, member 6 | Slc12a6 | 107723 | −1.80 | 0.00 | 0.84 |
| 1437035_PM_x_at | ring finger protein 14 | Rnf14 | 56736 | −1.68 | 0.00 | 5.05 |
| 1437062_PM_s_at | phytanoyl-CoA hydroxylase interacting | Phyhipl | 70911 | −1.69 | 0.00 | 8.67 |
| | protein-like | | | | | |
| 1437099_PM_x_at | predicted gene 11793 /// heterogeneous | Gm11793 /// | 637008 /// | −1.53 | 0.00 | 2.87 |
| | nuclear ribonucleoprotein F | Hnrnpf | 98758 | | | |
| 1437168_PM_at | splicing factor, arginine/serine-rich 13B | Sfrs13b | 272009 | −1.66 | 0.00 | 5.59 |
| 1437172_PM_x_at | hydroxyacyl-Coenzyme A dehydrogenase/ | Hadhb | 231086 | −1.86 | 0.00 | 16.38 |
| | 3-ketoacyl-Coenzyme A thiolase/enoyl- | | | | | |
| | Coenzyme A hydratase (trifunctional | | | | | |
| | protein), beta subunit | | | | | |
| 1437236_PM_a_at | zinc finger protein 110 | Zfp110 | 65020 | −1.77 | 0.00 | 2.48 |
| 1437278_PM_a_at | ubiquitin-like modifier activating enzyme 2 | Uba2 | 50995 | −1.50 | 0.00 | 11.02 |
| 1437325_PM_x_at | aldehyde dehydrogenase 18 family, | Aldh18a1 | 56454 | −1.51 | 0.00 | 4.55 |
| | member A1 | | | | | |
| 1437327_PM_x_at | enolase-phosphatase 1 | Enoph1 | 67870 | −1.62 | 0.00 | 9.39 |
| 1437455_PM_a_at | B-cell translocation gene 1, anti- | Btg1 /// | 100047353 /// | −1.55 | 0.00 | 4.34 |
| | proliferative /// similar to myocardial | LOC100047353 | 12226 | | | |
| | vascular inhibition factor | | | | | |
| 1437508_PM_at | trans-acting transcription factor 4 | Sp4 | 20688 | −1.54 | 0.00 | 6.91 |
| 1437525_PM_a_at | polymerase (RNA) III (DNA directed) | Polr3a | 218832 | −1.76 | 0.00 | 0.81 |
| | polypeptide A | | | | | |
| 1437526_PM_x_at | predicted gene 6159 /// heterogeneous | Gm6159 /// | 620521 /// | −1.61 | 0.00 | 6.07 |
| | nuclear ribonucleoprotein R | Hnrnpr | 74326 | | | |
| 1437528_PM_x_at | RIKEN cDNA A730017C20 gene | A730017C20Rik | 225583 | −2.06 | 0.00 | 12.35 |
| 1437671_PM_x_at | protease, serine, 23 | Prss23 | 76453 | −1.74 | 0.00 | 5.74 |
| 1437715_PM_x_at | apurinic/apyrimidinic endonuclease 1 | Apex1 | 11792 | −1.52 | 0.00 | 15.59 |
| 1437723_PM_s_at | Der1-like domain family, member 1 | Derl1 | 67819 | −1.51 | 0.00 | 8.38 |
| 1437837_PM_x_at | polymerase (DNA-directed), delta | Poldip3 | 73826 | −1.74 | 0.00 | 2.31 |
| | interacting protein 3 | | | | | |
| 1437845_PM_x_at | protein O-fucosyltransferase 2 | Pofut2 | 80294 | −1.98 | 0.00 | 7.81 |
| 1437878_PM_s_at | tetratricopeptide repeat domain 14 | Ttc14 | 67120 | −1.64 | 0.00 | 4.52 |
| 1437987_PM_at | — | — | — | −1.66 | 0.00 | 3.67 |
| 1438115_PM_a_at | solute carrier family 9 (sodium/hydrogen | Slc9a3r1 | 26941 | −1.85 | 0.00 | 12.04 |
| | exchanger), member 3 regulator 1 | | | | | |
| 1438171_PM_x_at | methyltransferase like 9 | Mettl9 | 59052 | −1.63 | 0.00 | 7.84 |
| 1438259_PM_at | — | — | — | −1.59 | 0.00 | 0.70 |
| 1438360_PM_x_at | predicted gene 5256 /// predicted pseudogene | Gm5256 /// | 11740 /// | −1.53 | 0.00 | 8.21 |
| | 5529 /// solute carrier family 25 (mitochondrial | Gm5529 /// | 383528 /// | | | |
| | carrier, adenine nucleotide translocator), | Slc25a5 | 433326 | | | |
| | member 5 | | | | | |
| 1438371_PM_x_at | DEAD (Asp-Glu-Ala-Asp) box polypeptide 5 | Ddx5 | 13207 | −1.52 | 0.00 | 8.62 |
| 1438506_PM_s_at | abl-interactor 1 | Abi1 | 11308 | −2.04 | 0.00 | 11.43 |
| 1438553_PM_x_at | RIKEN cDNA 4930453N24 gene | 4930453N24Rik | 67609 | −1.61 | 0.00 | 4.68 |
| 1438557_PM_x_at | aspartyl aminopeptidase | Dnpep | 13437 | −1.50 | 0.00 | 5.39 |
| 1438562_PM_a_at | protein tyrosine phosphatase, non- | Ptpn2 | 19255 | −1.74 | 0.00 | 4.64 |
| | receptor type 2 | | | | | |
| 1438624_PM_x_at | heparan sulfate (glucosamine) 3-O- | Hs3st2 | 195646 | −1.86 | 0.00 | 3.90 |
| | sulfotransferase 2 | | | | | |
| 1438634_PM_x_at | LIM and SH3 protein 1 | Lasp1 | 16796 | −1.50 | 0.00 | 4.82 |
| 1438653_PM_x_at | ataxin 10 | Atxn10 | 54138 | −1.69 | 0.00 | 11.56 |
| 1438786_PM_a_at | RIKEN cDNA 2610021A01 gene | 2610021A01Rik | 668572 | −1.90 | 0.00 | 8.21 |
| 1438803_PM_s_at | sorting nexin 16 | Snx16 | 74718 | −1.55 | 0.00 | 3.92 |
| 1438922_PM_x_at | predicted gene 5256 /// predicted pseudogene | Gm5256 /// | 11740 /// | −1.51 | 0.00 | 7.19 |
| | 5529 /// solute carrier family 25 | Gm5529 /// | 383528 /// | | | |
| | (mitochondrial carrier, adenine nucleotide | Slc25a5 | 433326 | | | |
| | translocator), member 5 | | | | | |
| 1438931_PM_s_at | similar to Sesn1 protein /// sestrin 1 | LOC100047324 /// | 100047324 /// | −1.97 | 0.00 | 12.74 |
| | | Sesn1 | 140742 | | | |
| 1438941_PM_x_at | adenosine monophosphate deaminase 2 | Ampd2 | 109674 | −1.76 | 0.00 | 2.86 |
| 1439036_PM_a_at | ATPase, Na+/K+ transporting, beta 1 | Atp1b1 | 11931 | −1.57 | 0.00 | 8.67 |
| | polypeptide | | | | | |
| 1439078_PM_at | kelch-like 4 (Drosophila) | Klhl4 | 237010 | −1.64 | 0.00 | 4.54 |
| 1439243_PM_x_at | COP9 (constitutive photomorphogenic) | Cops5 | 26754 | −1.56 | 0.00 | 0.97 |
| | homolog, subunit 5 (Arabidopsis thaliana) | | | | | |
| 1439249_PM_at | WW domain containing adaptor with | Wac | 225131 | −1.57 | 0.00 | 7.06 |
| | coiled-coil | | | | | |
| 1439255_PM_s_at | G protein-coupled receptor 137B /// G | Gpr137b /// | 100044979 /// | −1.61 | 0.00 | 4.97 |
| | protein-coupled receptor 137B, | Gpr137b-ps /// | 664862 /// | | | |
| | pseudogene /// similar to Gpr137b protein | LOC100044979 | 83924 | | | |
| 1439399_PM_a_at | small nucleolar RNA host gene | Snhg1 | 83673 | −1.62 | 0.00 | 10.35 |
| | (non-protein coding) 1 | | | | | |
| 1439403_PM_x_at | ring finger protein, LIM domain interacting | Rlim | 19820 | −1.58 | 0.00 | 14.01 |
| 1439424_PM_x_at | HERPUD family member 2 | Herpud2 | 80517 | −1.70 | 0.00 | 12.80 |
| 1439442_PM_x_at | tyrosyl-tRNA synthetase 2 (mitochondrial) | Yars2 | 70120 | −1.70 | 0.00 | 4.73 |
| 1439444_PM_x_at | transmembrane emp24-like trafficking | Tmed10 | 68581 | −1.52 | 0.00 | 9.86 |
| | protein 10 (yeast) | | | | | |
| 1439464_PM_s_at | testis expressed gene 10 | Tex10 | 269536 | −1.54 | 0.00 | 6.55 |
| 1439616_PM_at | — | — | — | −1.84 | 0.00 | 7.17 |
| 1439619_PM_at | transcription factor 12 | Tcf12 | 21406 | −1.55 | 0.00 | 1.73 |
| 1439627_PM_at | zinc finger protein of the cerebellum 1 | Zic1 | 22771 | −1.75 | 0.00 | 8.86 |
| 1439635_PM_at | regulator of G-protein signaling 9 | Rgs9 | 19739 | 1.54 | 0.00 | 1.38 |
| 1439651_PM_at | — | — | — | −1.51 | 0.00 | 8.25 |
| 1439697_PM_at | interleukin 1 receptor accessory protein | Il1rap | 16180 | −1.51 | 0.00 | 5.63 |
| 1439808_PM_at | interaction protein for cytohesin | Ipcef1 | 320495 | −1.55 | 0.00 | 12.57 |
| | exchange factors 1 | | | | | |
| 1439824_PM_at | choroidermia | Chm | 12662 | −1.66 | 0.00 | 7.62 |
| 1439906_PM_at | — | — | — | −1.74 | 0.00 | 10.65 |
| 1440177_PM_at | glutamate receptor, ionotropic, kainate 3 | Grik3 | 14807 | −1.55 | 0.00 | 6.73 |
| 1440204_PM_at | RIKEN cDNA 3110039M20 gene | 3110039M20Rik | 67293 | −1.66 | 0.00 | 2.19 |
| 1440810_PM_x_at | — | — | — | −1.54 | 0.00 | 0.69 |
| 1440816_PM_x_at | DEAD (Asp-Glu-Ala-Asp) box polypeptide 1 | Ddx1 | 104721 | −1.62 | 0.00 | 4.65 |
| 1441662_PM_at | cytochrome P450, family 4, subfamily x, | Cyp4x1 | 81906 | −1.50 | 0.00 | 4.74 |
| | polypeptide 1 | | | | | |
| 1441905_PM_x_at | small nuclear ribonucleoprotein N /// | Snrpn /// | 20646 /// | −1.70 | 0.00 | 6.70 |
| | SNRPN upstream reading frame | Snurf | 84704 | | | |
| 1441936_PM_x_at | — | — | — | −1.87 | 0.00 | 1.47 |
| 1442029_PM_at | KCNQ1 overlapping transcript 1 | Kcnq1ot1 | 63830 | −1.60 | 0.00 | 0.72 |
| 1442157_PM_at | — | — | — | −1.61 | 0.00 | 7.70 |
| 1442220_PM_at | — | — | — | −1.71 | 0.00 | 5.06 |
| 1442572_PM_at | — | — | — | 1.65 | 0.00 | 4.35 |
| 1443036_PM_at | zinc finger protein 804A | Zfp804a | 241514 | −1.61 | 0.00 | 4.07 |
| 1443773_PM_at | YLP motif containing 1 | Ylpm1 | 56531 | −1.90 | 0.00 | 9.50 |
| 1443790_PM_x_at | RIKEN cDNA 4930414L22 gene | 4930414L22Rik | 78108 | −1.76 | 0.00 | 1.09 |
| 1444082_PM_at | RIKEN cDNA A730017C20 gene | A730017C20Rik | 225583 | −1.50 | 0.00 | 1.90 |
| 1444159_PM_at | — | — | — | 1.52 | 0.00 | 0.82 |
| 1445824_PM_at | zinc finger protein 458 | Zfp458 | 238690 | −1.87 | 0.00 | 1.96 |
| 1446452_PM_at | — | — | — | 1.54 | 0.00 | 0.81 |
| 1447522_PM_s_at | tankyrase, TRF1-interacting ankyrin-related | Tnks2 | 74493 | −1.67 | 0.00 | 7.86 |
| | ADP-ribose polymerase 2 | | | | | |
| 1447694_PM_x_at | neogenin | Neo1 | 18007 | 4.17 | 0.00 | 0.47 |
| 1447725_PM_at | RIKEN cDNA C030034E14 gene | C030034E14Rik | 77469 | −1.63 | 0.00 | 8.43 |
| 1447816_PM_x_at | oxidoreductase NAD-binding domain | Oxnad1 | 218885 | −1.57 | 0.00 | 1.98 |
| | containing 1 | | | | | |
| 1447837_PM_x_at | polymerase (DNA directed), eta (RAD 30 related) | Polh | 80905 | −1.62 | 0.00 | 2.87 |
| 1447861_PM_x_at | Meis homeobox 2 | Meis2 | 17536 | −1.64 | 0.00 | 10.04 |
| 1447868_PM_x_at | glutaredoxin 3 | Glrx3 | 30926 | −1.56 | 0.00 | 7.84 |
| 1447877_PM_x_at | DNA methyltransferase (cytosine-5) 1 | Dnmt1 | 13433 | −1.58 | 0.00 | 12.41 |
| 1447898_PM_s_at | splicing factor, arginine/serine-rich 6 | Sfrs6 | 67996 | −1.51 | 0.00 | 0.38 |
| 1447919_PM_x_at | NADH dehydrogenase (ubiquinone) 1, | Ndufab1 | 70316 | −1.51 | 0.00 | 8.12 |
| | alpha/beta subcomplex, 1 | | | | | |
| 1447951_PM_at | — | — | — | 1.54 | 0.00 | 1.21 |
| 1447985_PM_s_at | ankyrin repeat and IBR domain containing 1 | Ankib1 | 70797 | −1.63 | 0.00 | 5.37 |
| 1448103_PM_s_at | non-POU-domain-containing, octamer | Nono | 53610 | −1.55 | 0.00 | 9.03 |
| | binding protein | | | | | |
| 1448272_PM_at | B-cell translocation gene 2, anti-proliferative | Btg2 | 12227 | 2.49 | 0.00 | 0.89 |
| 1448557_PM_at | family with sequence similarity 13, member C | Fam13c | 71721 | −1.65 | 0.00 | 5.50 |
| 1448830_PM_at | dual specificity phosphatase 1 | Dusp1 | 19252 | 1.51 | 0.00 | 3.26 |
| 1448865_PM_at | hydroxysteroid (17-beta) dehydrogenase 7 | Hsd17b7 | 15490 | 1.53 | 0.00 | 0.93 |
| 1448950_PM_at | interleukin 1 receptor, type I | Il1r1 | 16177 | 1.54 | 0.00 | 1.70 |
| 1448986_PM_x_at | deoxyribonuclease II alpha | Dnase2a | 13423 | −1.62 | 0.00 | 5.22 |
| 1449676_PM_at | — | — | — | −1.59 | 0.00 | 8.94 |
| 1449686_PM_s_at | sterol carrier protein 2, liver | Scp2 | 20280 | −1.56 | 0.00 | 12.17 |
| 1450744_PM_at | elongation factor RNA polymerase II 2 | Ell2 | 192657 | −1.54 | 0.00 | 5.16 |
| 1450896_PM_at | Rho GTPase activating protein 5 | Arhgap5 | 11855 | −1.80 | 0.00 | 9.65 |
| 1451146_PM_at | zinc finger protein 386 (Kruppel-like) | Zfp386 | 56220 | −1.53 | 0.00 | 4.35 |
| 1451313_PM_a_at | RIKEN cDNA 1110067D22 gene | 1110067D22Rik | 216551 | −1.58 | 0.00 | 5.47 |
| 1452039_PM_a_at | Brca1 associated protein 1 | Bap1 | 104416 | −1.73 | 0.00 | 6.19 |
| 1452190_PM_at | prolylcarboxypeptidase (angiotensinase C) | Prcp | 72461 | 1.53 | 0.00 | 3.70 |
| 1452281_PM_at | son of sevenless homolog 2 (Drosophila) | Sos2 | 20663 | −1.56 | 0.00 | 9.51 |
| 1452318_PM_a_at | heat shock protein 1B | Hspa1b | 15511 | −1.57 | 0.00 | 1.21 |
| 1452426_PM_x_at | — | — | — | −1.56 | 0.00 | 1.47 |
| 1452593_PM_a_at | transcription elongation factor B (SIII), | Tceb1 | 67923 | −1.52 | 0.00 | 3.85 |
| | polypeptide 1 | | | | | |
| 1452700_PM_s_at | kelch repeat and BTB (POZ) domain | Kbtbd7 | 211255 | −1.52 | 0.00 | 6.40 |
| | containing 7 | | | | | |
| 1452758_PM_s_at | eukaryotic translation initiation | Eif4g2 | 13690 | −1.91 | 0.00 | 6.47 |
| | factor 4, gamma 2 | | | | | |
| 1453006_PM_at | fibroblast growth factor binding protein 3 | Fgfbp3 | 72514 | −1.58 | 0.00 | 0.80 |
| 1453070_PM_at | protocadherin 17 | Pcdh17 | 219228 | −1.53 | 0.00 | 11.72 |
| 1453134_PM_at | phosphatidylinositol 3-kinase, catalytic, | Pik3ca | 18706 | −1.62 | 0.00 | 5.04 |
| | alpha polypeptide | | | | | |
| 1453782_PM_at | ankyrin repeat domain 33B | Ankrd33b | 67434 | −1.64 | 0.00 | 13.91 |
| 1454642_PM_a_at | COMM domain containing 3 | Commd3 | 12238 | −1.50 | 0.00 | 10.74 |
| 1454688_PM_x_at | transmembrane emp24-like trafficking | Tmed10 | 68581 | −1.52 | 0.00 | 7.52 |
| | protein 10 (yeast) | | | | | |
| 1454725_PM_at | transformer 2 alpha homolog (Drosophila) | Tra2a | 101214 | −1.65 | 0.00 | 6.68 |
| 1454765_PM_at | general transcription factor IIIC, | Gtf3c3 | 98488 | −1.82 | 0.00 | 4.72 |
| | polypeptide 3 | | | | | |
| 1454805_PM_at | Wilms' tumour 1-associating protein | Wtap | 60532 | −1.57 | 0.00 | 4.38 |
| 1454816_PM_at | retinitis pigmentosa 2 homolog (human) | Rp2h | 19889 | −1.51 | 0.00 | 2.56 |
| 1454842_PM_a_at | UDP-GalNAc:betaGlcNAc beta 1,3- | B3galnt2 | 97884 | −1.86 | 0.00 | 1.45 |
| | galactosaminyltransferase, polypeptide 2 | | | | | |
| 1454966_PM_at | integrin alpha 8 | Itga8 | 241226 | −1.51 | 0.00 | 0.58 |
| 1455105_PM_at | protein tyrosine phosphatase, non-receptor | Ptpn12 | 19248 | −1.54 | 0.00 | 7.55 |
| | type 12 | | | | | |
| 1455234_PM_at | UDP-Gal:betaGlcNAc beta 1,3- | B3galt1 | 26877 | −1.50 | 0.00 | 9.76 |
| | galactosyltransferase, polypeptide 1 | | | | | |
| 1455317_PM_at | enhancer of polycomb homolog 2 (Drosophila) | Epc2 | 227867 | −1.56 | 0.00 | 8.35 |
| 1455337_PM_at | FYVE, RhoGEF and PH domain containing 4 | Fgd4 | 224014 | −1.52 | 0.00 | 4.79 |
| 1455470_PM_x_at | LIM and SH3 protein 1 | Lasp1 | 16796 | −1.56 | 0.00 | 8.29 |
| 1455603_PM_at | — | — | — | −1.71 | 0.00 | 12.08 |
| 1455750_PM_at | Ral GTPase activating protein, alpha subunit | Ralgapa2 | 241694 | −1.74 | 0.00 | 3.21 |
| | 2 (catalytic) | | | | | |
| 1455796_PM_x_at | olfactomedin 1 | Olfm1 | 56177 | −1.65 | 0.00 | 10.70 |
| 1455809_PM_x_at | resistance to inhibitors of cholinesterase | Ric8 | 101489 | −1.56 | 0.00 | 2.70 |
| | 8 homolog (C. elegans) | | | | | |
| 1455816_PM_a_at | potassium channel tetramerisation | Kctd3 | 226823 | −1.88 | 0.00 | 6.75 |
| | domain containing 3 | | | | | |
| 1455822_PM_x_at | surfeit gene 4 | Surf4 | 20932 | −1.56 | 0.00 | 8.11 |
| 1455883_PM_a_at | leucine rich repeat transmembrane neuronal 1 | Lrrtm1 | 74342 | −1.60 | 0.00 | 7.21 |
| 1455928_PM_x_at | leucine-zipper-like transcriptional regulator, 1 | Lztr1 | 66863 | −2.01 | 0.00 | 16.54 |
| 1455940_PM_x_at | WD repeat domain 6 | Wdr6 | 83669 | −1.50 | 0.00 | 13.77 |
| 1455978_PM_a_at | matrilin 2 | Matn2 | 17181 | −2.07 | 0.00 | 7.48 |
| 1456032_PM_x_at | predicted gene 8203 /// H2A histone | Gm8203 /// | 51788 /// | −1.53 | 0.00 | 8.50 |
| | family, member Z | H2afz | 666634 | | | |
| 1456036_PM_x_at | glutathione S-transferase omega 1 | Gsto1 | 14873 | −1.64 | 0.00 | 8.63 |
| 1456041_PM_at | sorting nexin 16 | Snx16 | 74718 | −1.78 | 0.00 | 8.08 |
| 1456056_PM_a_at | DNA segment, Chr 6, Wayne State | D6Wsu116e | 28006 | −1.74 | 0.00 | 12.73 |
| | University 116, expressed | | | | | |
| 1456071_PM_a_at | cytochrome c, somatic /// predicted | Cycs /// | 13063 /// | −1.64 | 0.00 | 7.89 |
| | gene 10053 | Gm10053 | 672195 | | | |
| 1456089_PM_at | tripartite motif-containing 23 | Trim23 | 81003 | −1.71 | 0.00 | 12.20 |
| 1456107_PM_x_at | elongation factor Tu GTP binding | Eftud2 | 20624 | −1.61 | 0.00 | 11.15 |
| | domain containing 2 | | | | | |
| 1456108_PM_x_at | ring finger protein 112 | Rnf112 | 22671 | −1.57 | 0.00 | 4.61 |
| 1456226_PM_x_at | discoidin domain receptor family, member 1 | Ddr1 | 12305 | −1.58 | 0.00 | 10.81 |
| 1456527_PM_at | HECT, C2 and WW domain containing E3 | Hecw1 | 94253 | −1.55 | 0.00 | 2.19 |
| | ubiquitin protein ligase 1 | | | | | |
| 1456542_PM_s_at | glutaminyl-tRNA synthase (glutamine- | Qrsl1 | 76563 | −1.67 | 0.00 | 9.61 |
| | hydrolyzing)-like 1 | | | | | |
| 1456577_PM_x_at | pitrilysin metallepetidase 1 | Pitrm1 | 69617 | −1.68 | 0.00 | 6.44 |
| 1456728_PM_x_at | aconitase 1 | Aco1 | 11428 | −2.14 | 0.00 | 11.03 |
| 1456739_PM_x_at | armadillo repeat containing, X-linked 2 | Armcx2 | 67416 | −1.76 | 0.00 | 6.04 |
| 1456901_PM_at | a disintegrin-like and metallopeptidase | Adamts20 | 223838 | −1.69 | 0.00 | 3.79 |
| | (reprolysin type) with thrombospondin | | | | | |
| | type 1 motif, 20 | | | | | |
| 1457069_PM_at | activating signal cointegrator 1 | Ascc3 | 77987 | −1.61 | 0.00 | 4.44 |
| | complex subunit 3 | | | | | |
| 1457257_PM_x_at | poliovirus receptor-related 3 | Pvrl3 | 58998 | −2.12 | 0.00 | 5.31 |
| 1457273_PM_at | odd Oz/ten-m homolog 2 (Drosophila) | Odz2 | 23964 | −1.53 | 0.00 | 8.87 |
| 1457494_PM_at | — | — | — | −1.53 | 0.00 | 4.08 |
| 1457635_PM_s_at | nuclear receptor subfamily 3, group C, | Nr3c1 | 14815 | −1.58 | 0.00 | 3.79 |
| | member 1 | | | | | |
| 1458051_PM_at | RIKEN cDNA A230048O21 gene | A230048O21Rik | 320959 | 1.53 | 0.00 | 2.42 |
| 1458403_PM_at | TRAF2 and NCK interacting kinase | Tnik | 665113 | −1.58 | 0.00 | 13.28 |
| 1458639_PM_at | — | — | — | 1.51 | 0.00 | 0.05 |
| 1459409_PM_at | — | — | — | 1.71 | 0.00 | 3.33 |
| 1459783_PM_s_at | cappuccino | Cno | 117197 | −1.77 | 0.00 | 4.41 |
| 1459806_PM_x_at | mitochondrial ribosomal protein S23 | Mrps23 | 64656 | −1.56 | 0.00 | 5.08 |
| 1459835_PM_s_at | DnaJ (Hsp40) homolog, subfamily A, member 1 | Dnaja1 | 15502 | −1.60 | 0.00 | 9.25 |
| 1459971_PM_at | potassium channel, subfamily T, member 2 | Kcnt2 | 240776 | −1.73 | 0.00 | 9.72 |
| 1460449_PM_at | ankyrin repeat and sterile alpha motif | Anks1b | 77531 | −1.56 | 0.00 | 2.44 |
| | domain containing 1B | | | | | |
| 1460455_PM_at | ubiquitin protein ligase E3 component | Ubr3 | 68795 | −1.63 | 0.00 | 9.93 |
| | n-recognin 3 | | | | | |
| 1460710_PM_at | adenosine A2a receptor | Adora2a | 11540 | 1.66 | 0.00 | 3.83 |

100DMF-vs-100MMF

| | | | | | | |
|---|---|---|---|---|---|---|
| 1424257_PM_at | cyclin-dependent kinase 7 | Cdk7 | 12572 | 1.73 | 0.00 | 8.00 |
| 1433836_PM_a_at | RIKEN cDNA 8430408G22 gene | 8430408G22Rik | 213393 | 1.78 | 0.00 | 7.17 |
| 1435146_PM_s_at | cell adhesion molecule 2 | Cadm2 | 239857 | 1.50 | 0.00 | 2.12 |
| 1436885_PM_a_at | calcium homeostasis endoplasmic | Cherp | 27967 | 1.53 | 0.00 | 0.15 |
| | reticulum protein | | | | | |
| 1437528_PM_x_at | RIKEN cDNA A730017C20 gene | A730017C20Rik | 225583 | 1.56 | 0.00 | 4.17 |
| 1447694_PM_x_at | neogenin | Neo1 | 18007 | −3.98 | 0.00 | 0.10 |
| 1448002_PM_x_at | RIKEN cDNA 2610001J05 gene | 2610001J05Rik | 66520 | −4.35 | 0.00 | 1.39 |
| 1456728_PM_x_at | aconitase 1 | Aco1 | 11428 | 1.56 | 0.00 | 2.46 |

CEREBELLUM
100DMF-vs-Veh

| | | | | | | |
|---|---|---|---|---|---|---|
| 1419435_PM_at | aldehyde oxidase 1 | Aox1 | 11761 | 1.60 | 0.00 | 4.51 |
| 1419892_PM_at | RIKEN cDNA 1110021J02 gene | 1110021J02Rik | 68597 | 1.54 | 0.00 | 0.57 |
| 1420481_PM_at | cyclin M3 | Cnnm3 | 94218 | 1.62 | 0.00 | 0.68 |
| 1421622_PM_a_at | Rap guanine nucleotide exchange factor | Rapgef4 | 56508 | −1.55 | 0.00 | 1.34 |
| | (GEF) 4 | | | | | |
| 1437277_PM_x_at | transglutaminase 2, C polypeptide | Tgm2 | 21817 | 1.51 | 0.00 | 0.03 |
| 1441228_PM_at | apolipoprotein L domain containing 1 | Apold1 | 381823 | 1.85 | 0.00 | 1.00 |
| 1452473_PM_at | proline rich 15 | Prr15 | 78004 | 1.80 | 0.00 | 5.62 |
| 1457266_PM_at | — | — | — | 1.51 | 0.00 | 1.12 |

100MMF-vs-Veh

| | | | | | | |
|---|---|---|---|---|---|---|
| 1416965_PM_at | proprotein convertase subtilisin/kexin | Pcsk1n | 30052 | 1.50 | 0.00 | 0.06 |
| | type 1 inhibitor | | | | | |
| 1419435_PM_at | aldehyde oxidase 1 | Aox1 | 11761 | 1.54 | 0.00 | 3.91 |
| 1419892_PM_at | RIKEN cDNA 1110021J02 gene | 1110021J02Rik | 68597 | 1.56 | 0.00 | 1.55 |
| 1421974_PM_at | leucine rich repeat containing 50 | Lrrc50 | 68270 | 1.61 | 0.00 | 1.16 |
| 1423457_PM_at | solute carrier family 35, member A5 | Slc35a5 | 74102 | −1.50 | 0.00 | 0.50 |
| 1425079_PM_at | transmembrane 6 superfamily member 2 | Tm6sf2 | 107770 | 1.85 | 0.00 | 4.66 |
| 1439754_PM_at | SRY-box containing gene 12 | Sox12 | 20667 | 1.67 | 0.00 | 0.89 |
| 1441228_PM_at | apolipoprotein L domain containing 1 | Apold1 | 381823 | 1.95 | 0.00 | 2.91 |
| 1442605_PM_at | — | — | — | −1.75 | 0.00 | 2.50 |
| 1442912_PM_at | RIKEN cDNA 9430064I24 gene | 9430064I24Rik | 100327266 | 1.86 | 0.00 | 2.70 |
| 1446131_PM_at | — | — | — | 1.78 | 0.00 | 0.59 |
| 1446929_PM_at | RIKEN cDNA D130062J21 gene | D130062J21Rik | 100038651 | 1.66 | 0.00 | 11.83 |
| 1450821_PM_at | K(lysine) acetyltransferase 2B | Kat2b | 18519 | −1.68 | 0.00 | 1.38 |
| 1452473_PM_at | proline rich 15 | Prr15 | 78004 | 1.53 | 0.00 | 1.65 |
| 1456962_PM_at | contactin 2 | Cntn2 | 21367 | 1.98 | 0.00 | 1.01 |

100DMF-vs-100MMF

None

SPINAL CORD
100DMF-vs-Veh

| | | | | | | |
|---|---|---|---|---|---|---|
| 1433837_PM_at | RIKEN cDNA 8430408G22 gene | 8430408G22Rik | 213393 | 1.73 | 0.00 | 1.47 |

100MMF-vs-Veh

None

100DMF-vs-100MMF

| | | | | | | |
|---|---|---|---|---|---|---|
| 1425459_PM_at | myotubularin related protein 2 | Mtmr2 | 77116 | 1.57 | 0.00 | 1.16 |
| 1430384_PM_at | transducin-like enhancer of split 4, | Tle4 | 21888 | −1.55 | 0.00 | 0.45 |
| | homolog of Drosophila E(spl) | | | | | |
| 1433836_PM_a_at | RIKEN cDNA 8430408G22 gene | 8430408G22Rik | 213393 | 1.92 | 0.00 | 3.02 |
| 1433837_PM_at | RIKEN cDNA 8430408G22 gene | 8430408G22Rik | 213393 | 1.65 | 0.00 | 0.66 |

SPLEEN
100DMF-vs-Veh

| | | | | | | |
|---|---|---|---|---|---|---|
| 1416416_PM_x_at | glutathione S-transferase, mu 1 | Gstm1 | 14862 | 1.57 | 0.00 | 11.10 |
| 1419435_PM_at | aldehyde oxidase 1 | Aox1 | 11761 | 1.66 | 0.00 | 5.79 |
| 1423436_PM_at | glutathione S-transferase, alpha 3 | Gsta3 | 14859 | 1.86 | 0.00 | 7.90 |
| 1448330_PM_at | glutathione S-transferase, mu 1 | Gstm1 | 14862 | 1.61 | 0.00 | 10.42 |

100MMF-vs-Veh

| | | | | | | |
|---|---|---|---|---|---|---|
| 1417307_PM_at | dystrophin, muscular dystrophy | Dmd | 13405 | −1.56 | 0.00 | 0.49 |
| 1417876_PM_at | Fc receptor, IgG, high affinity I | Fcgr1 | 14129 | −1.84 | 0.00 | 4.24 |
| 1417898_PM_a_at | granzyme A | Gzma | 14938 | −1.64 | 0.00 | 0.82 |
| 1419043_PM_a_at | interferon inducible GTPase 1 | Iigp1 | 60440 | −1.72 | 0.00 | 2.80 |
| 1419491_PM_at | defensin beta 1 | Defb1 | 13214 | 1.55 | 0.00 | 0.14 |
| 1419561_PM_at | chemokine (C-C motif) ligand 3 | Ccl3 | 20302 | −1.82 | 0.00 | 0.28 |
| 1419721_PM_at | niacin receptor 1 | Niacr1 | 80885 | −1.91 | 0.00 | 4.45 |
| 1420723_PM_at | vanin 3 | Vnn3 | 26464 | −1.59 | 0.00 | 1.14 |
| 1421492_PM_at | hematopoietic prostaglandin D synthase | Hpgds | 54486 | −1.55 | 0.00 | 7.16 |
| 1421974_PM_at | leucine rich repeat containing 50 | Lrrc50 | 68270 | 1.53 | 0.00 | 3.49 |
| 1422013_PM_at | C-type lectin domain family 4, member a2 | Clec4a2 | 26888 | −1.50 | 0.00 | 1.76 |
| 1422412_PM_x_at | eosinophil-associated, ribonuclease A | Ear3 | 53876 | −1.73 | 0.00 | 2.12 |
| | family, member 3 | | | | | |
| 1422957_PM_at | chemokine (C-C motif) receptor 3 | Ccr3 | 12771 | −2.37 | 0.00 | 1.21 |
| 1424187_PM_at | coiled-coil domain containing 80 | Ccdc80 | 67896 | −1.64 | 0.00 | 0.40 |
| 1424832_PM_at | CD300 molecule-like family member d | Cd300ld | 217305 | −1.85 | 0.00 | 5.24 |
| 1424998_PM_at | EGF-like module containing, mucin-like, | Emr4 | 52614 | −1.75 | 0.00 | 1.58 |
| | hormone receptor-like sequence 4 | | | | | |
| 1425282_PM_at | ring finger protein 144B | Rnf144b | 218215 | −1.71 | 0.00 | 1.50 |
| 1425406_PM_at | C-type lectin domain family 4, member a2 | Clec4a2 | 26888 | −1.64 | 0.00 | 3.72 |
| 1425951_PM_a_at | C-type lectin domain family 4, member n | Clec4n | 56620 | −1.81 | 0.00 | 3.60 |
| 1426288_PM_at | low density lipoprotein receptor-related | Lrp4 | 228357 | −1.51 | 0.00 | 0.74 |
| | protein 4 | | | | | |
| 1429277_PM_at | — | — | — | −1.60 | 0.00 | 3.34 |
| 1429954_PM_at | C-type lectin domain family 4, member a3 | Clec4a3 | 73149 | −1.58 | 0.00 | 1.76 |
| 1430056_PM_at | ubinuclein 2 | Ubn2 | 320538 | 1.64 | 0.00 | 1.26 |
| 1430416_PM_at | RIKEN cDNA 4931431B13 gene | 4931431B13Rik | 70973 | −1.94 | 0.00 | 0.65 |
| 1431225_PM_at | — | — | — | 1.58 | 0.00 | 3.06 |
| 1431691_PM_a_at | RAB31, member RAS oncogene family | Rab31 | 106572 | −1.62 | 0.00 | 2.81 |
| 1434343_PM_at | RIKEN cDNA 5730403M16 gene | 5730403M16Rik | 232853 | 1.51 | 0.00 | 2.88 |
| 1434583_PM_at | transmembrane protein 26 | Tmem26 | 327766 | −1.67 | 0.00 | 3.33 |
| 1437390_PM_x_at | syntaxin 1A (brain) | Stx1a | 20907 | 1.53 | 0.00 | 9.27 |
| 1438272_PM_at | CUB and Sushi multiple domains 3 | Csmd3 | 239420 | −1.80 | 0.00 | 0.37 |
| 1438321_PM_x_at | family with sequence similarity 63, member A | Fam63a | 75007 | 1.54 | 0.00 | 1.08 |
| 1439831_PM_at | — | — | — | −1.51 | 0.00 | 3.65 |
| 1442323_PM_at | — | — | — | 1.51 | 0.00 | 0.56 |
| 1442339_PM_at | stefin A2 like 1 | Stfa2l1 | 268885 | −2.07 | 0.00 | 0.42 |
| 1444566_PM_at | Uncoupling protein 2 (mitochondrial, | Ucp2 | 22228 | 1.54 | 0.00 | 2.59 |
| | proton carrier) | | | | | |
| 1447204_PM_at | — | — | — | −1.95 | 0.00 | 1.95 |
| 1447878_PM_s_at | fibroblast growth factor receptor-like | Fgfrl1 /// | 100046239 /// | 1.53 | 0.00 | 1.19 |
| | 1 /// similar to fibroblast growth factor | LOC100046239 | 116701 | | | |
| | receptor 5 beta | | | | | |
| 1449653_PM_at | — | — | — | −1.57 | 0.00 | 2.86 |
| 1450047_PM_at | heparan sulfate 6-O-sulfotransferase 2 | Hs6st2 | 50786 | −1.75 | 0.00 | 4.10 |
| 1450967_PM_at | protein tyrosine phosphatase-like A domain | Ptplad2 | 66775 | −1.55 | 0.00 | 0.32 |
| | containing 2 | | | | | |
| 1453540_PM_at | RIKEN cDNA 5430404G13 gene | 5430404G13Rik | 74502 | −1.55 | 0.00 | 1.46 |
| 1457306_PM_at | — | — | — | 2.12 | 0.00 | 2.45 |

100DMF-vs-100MMF

| | | | | | | |
|---|---|---|---|---|---|---|
| 1415989_PM_at | vascular cell adhesion molecule 1 | Vcam1 | 22329 | 1.50 | 0.00 | 1.64 |
| 1417307_PM_at | dystrophin, muscular dystrophy | Dmd | 13405 | 1.59 | 0.00 | 1.03 |
| 1417561_PM_at | apolipoprotein C-I | Apoc1 | 11812 | 1.74 | 0.00 | 1.74 |
| 1417876_PM_at | Fc receptor, IgG, high affinity I | Fcgr1 | 14129 | 1.61 | 0.00 | 0.71 |
| 1418243_PM_at | ficolin A | Fcna | 14133 | 1.57 | 0.00 | 2.58 |
| 1419561_PM_at | chemokine (C-C motif) ligand 3 | Ccl3 | 20302 | 1.81 | 0.00 | 0.16 |
| 1419627_PM_s_at | C-type lectin domain family 4, member n | Clec4n | 56620 | 1.52 | 0.00 | 3.55 |
| 1419721_PM_at | niacin receptor 1 | Niacr1 | 80885 | 1.65 | 0.00 | 0.80 |
| 1420249_PM_s_at | chemokine (C-C motif) ligand 6 | Ccl6 | 20305 | 1.88 | 0.00 | 5.86 |
| 1420250_PM_at | — | — | — | 1.50 | 0.00 | 0.80 |
| 1420723_PM_at | vanin 3 | Vnn3 | 26464 | 1.54 | 0.00 | 0.24 |
| 1421534_PM_at | — | — | — | 1.57 | 0.00 | 4.61 |
| 1421802_PM_at | eosinophil-associated, ribonuclease A | Ear1 | 13586 | 3.89 | 0.00 | 0.76 |
| | family, member 1 | | | | | |
| 1422089_PM_at | natural cytotoxicity triggering receptor 1 | Ncr1 | 17086 | 1.54 | 0.00 | 2.50 |
| 1422411_PM_s_at | eosinophil-associated, ribonuclease A | Ear1 /// | 13586 /// | 1.79 | 0.00 | 2.86 |
| | family, member 1 /// eosinophil-associated, | Ear12 /// | 13587 /// | | | |
| | ribonuclease A family, member 12 /// | Ear2 /// | 503845 /// | | | |
| | eosinophil-associated, ribonuclease A | Ear3 | 53876 | | | |
| | family, member 2 /// eosinophil-associated, | | | | | |
| | ribonuclease A family, member 3 | | | | | |
| 1422412_PM_x_at | eosinophil-associated, ribonuclease A | Ear3 | 53876 | 1.80 | 0.00 | 3.04 |
| | family, member 3 | | | | | |
| 1422584_PM_at | superkiller viralicidic activity 2-like | Skiv2l | 108077 | −1.52 | 0.00 | 1.93 |
| | (S. cerevisiae) | | | | | |
| 1422957_PM_at | chemokine (C-C motif) receptor 3 | Ccr3 | 12771 | 2.25 | 0.00 | 0.45 |
| 1424187_PM_at | coiled-coil domain containing 80 | Ccdc80 | 67896 | 1.74 | 0.00 | 1.84 |
| 1424766_PM_at | excision repair cross-complementing | Ercc61 | 236930 | −1.66 | 0.00 | 1.74 |
| | rodent repair deficiency complementation | | | | | |
| | group 6 - like | | | | | |
| 1424832_PM_at | CD300 molecule-like family member d | Cd300ld | 217305 | 1.82 | 0.00 | 4.75 |
| 1424998_PM_at | EGF-like module containing, mucin-like, | Emr4 | 52614 | 1.77 | 0.00 | 1.75 |
| | hormone receptor-like sequence 4 | | | | | |
| 1425282_PM_at | ring finger protein 144B | Rnf144b | 218215 | 1.86 | 0.00 | 3.66 |
| 1425639_PM_at | ArfGAP with dual PH domains 2 | Adap2 | 216991 | 1.60 | 0.00 | 4.91 |
| 1425951_PM_a_at | C-type lectin domain family 4, member n | Clec4n | 56620 | 1.83 | 0.00 | 3.90 |
| 1426571_PM_at | anoctamin 1, calcium activated chloride | Ano1 | 101772 | 1.70 | 0.00 | 0.51 |
| | channel | | | | | |
| 1427345_PM_a_at | sulfotransferase family 1A, phenol- | Sult1a1 | 20887 | 1.51 | 0.00 | 0.97 |
| | preferring, member 1 | | | | | |
| 1429277_PM_at | — | — | — | 1.59 | 0.00 | 3.17 |
| 1430056_PM_at | ubinuclein 2 | Ubn2 | 320538 | −1.62 | 0.00 | 1.00 |
| 1430416_PM_at | RIKEN cDNA 4931431B13 gene | 4931431B13Rik | 70973 | 2.18 | 0.00 | 2.93 |
| 1430700_PM_a_at | phospholipase A2, group VII (platelet- | Pla2g7 | 27226 | 1.51 | 0.00 | 0.51 |
| | activating factor acetylhydrolase, plasma) | | | | | |
| 1431691_PM_a_at | RAB31, member RAS oncogene family | Rab31 | 106572 | 1.73 | 0.00 | 4.64 |
| 1431724_PM_a_at | purinergic receptor P2Y, G-protein | P2ry12 | 70839 | 1.66 | 0.00 | 0.50 |
| | coupled 12 | | | | | |
| 1434583_PM_at | transmembrane protein 26 | Tmem26 | 327766 | 1.57 | 0.00 | 1.50 |
| 1435094_PM_at | potassium inwardly-rectifying channel, | Kcnj16 | 16517 | 1.69 | 0.00 | 1.72 |
| | subfamily J, member 16 | | | | | |
| 1436003_PM_at | Vascular cell adhesion molecule 1 | Vcam1 | 22329 | 1.62 | 0.00 | 4.12 |
| 1436739_PM_at | angiotensin II receptor, type 1a | Agtr1a | 11607 | 1.61 | 0.00 | 1.01 |
| 1438321_PM_x_at | family with sequence similarity 63, member A | Fam63a | 75007 | −1.52 | 0.00 | 0.69 |
| 1439327_PM_at | collagen and calcium binding EGF domains 1 | Ccbe1 | 320924 | 2.07 | 0.00 | 2.38 |
| 1442323_PM_at | — | — | — | −1.55 | 0.00 | 1.30 |
| 1444189_PM_at | — | — | — | 1.71 | 0.00 | 2.44 |
| 1445025_PM_at | expressed sequence AU015536 | AU015536 | 101232 | 1.55 | 0.00 | 0.21 |
| 1445027_PM_at | cerebellar degeneration-related protein 2-like | Cdr2l | 237988 | 1.57 | 0.00 | 0.61 |
| 1445379_PM_at | — | — | — | −1.51 | 0.00 | 0.05 |
| 1449846_PM_at | eosinophil-associated, ribonuclease A | Ear2 | 13587 | 1.51 | 0.00 | 0.88 |
| | family, member 2 | | | | | |
| 1450047_PM_at | heparan sulfate 6-O-sulfotransferase 2 | Hs6st2 | 50786 | 1.64 | 0.00 | 2.39 |
| 1450296_PM_at | killer cell lectin-like receptor subfamily | Klrb1a | 17057 | 1.78 | 0.00 | 2.06 |
| | B member 1A | | | | | |
| 1450967_PM_at | protein tyrosine phosphatase-like A | Ptplad2 | 66775 | 1.56 | 0.00 | 0.59 |
| | domain containing 2 | | | | | |
| 1451314_PM_a_at | vascular cell adhesion molecule 1 | Vcam1 | 22329 | 1.61 | 0.00 | 5.51 |
| 1455123_PM_at | suppression of tumorigenicity 18 | St18 | 240690 | 1.75 | 0.00 | 3.71 |
| 1458742_PM_at | latrophilin 3 | Lphn3 | 319387 | 1.60 | 0.00 | 0.37 |
| 1459713_PM_s_at | anoctamin 1, calcium activated chloride | Ano1 | 101772 | 1.55 | 0.00 | 4.04 |
| | channel | | | | | |

LYMPH NODE
100DMF-vs-Veh

| | | | | | | |
|---|---|---|---|---|---|---|
| 1419413_PM_at | chemokine (C-C motif) ligand 17 | Ccl17 | 20295 | −1.57 | 0.00 | 1.47 |
| 1420249_PM_s_at | chemokine (C-C motif) ligand 6 | Ccl6 | 20305 | 1.66 | 0.00 | 2.62 |
| 1422860_PM_at | neurotensin | Nts | 67405 | 2.72 | 0.00 | 2.59 |
| 1423627_PM_at | NAD(P)H dehydrogenase, quinone 1 | Nqo1 | 18104 | 1.95 | 0.00 | 13.06 |

100MMF-vs-Veh

| | | | | | | |
|---|---|---|---|---|---|---|
| 1421037_PM_at | neuronal PAS domain protein 2 | Npas2 | 18143 | 1.75 | 0.00 | 2.96 |
| 1423627_PM_at | NAD(P)H dehydrogenase, quinone 1 | Nqo1 | 18104 | 1.74 | 0.00 | 8.85 |
| 1460364_PM_at | general transcription factor II I repeat | Gtf2ird1 | 57080 | 1.56 | 0.00 | 4.71 |
| | domain-containing 1 | | | | | |

100DMF-vs-100MMF

None

### APPENDIX D

### EAE Single Dose Gene Lists

| | | | | | |
|---|---|---|---|---|---|
| | Gene | Entrez | | p. | |
| Gene Title | Symbol | Gene | FC | value | lods |

WHOLE BLOOD
100DMF-vs-Veh, 7 h

| | | | | | | |
|---|---|---|---|---|---|---|
| 1418649_PM_at | EGL nine homolog 3 | Egln3 | 112407 | 1.82 | 0.00 | 0.57 |
| | (C. elegans) | | | | | |
| 1436545_PM_at | deltex 4 homolog | Dtx4 | 207521 | 1.72 | 0.00 | 3.57 |
| | (Drosophila) | | | | | |
| 1449036_PM_at | ring finger protein 128 | Rnf128 | 66889 | 1.62 | 0.00 | 3.50 |

100DMF-vs-Veh, 12 h

None

100MMF-vs-Veh, 7 h

| | | | | | | |
|---|---|---|---|---|---|---|
| 1422532_PM_at | xeroderma pigmentosum, | Xpc | 22591 | 1.59 | 0.00 | 1.04 |
| | complementation group C | | | | | |
| 1422804_PM_at | serine (or cysteine) | Serpinb6b | 20708 | −1.64 | 0.00 | 0.02 |
| | peptidase inhibitor, | | | | | |
| | clade B, member 6b | | | | | |
| 1425417_PM_x_at | killer cell lectin-like | Klra8 | 16639 | −2.10 | 0.00 | 0.40 |
| | receptor, subfamily A, | | | | | |
| | member 8 | | | | | |
| 1425436_PM_x_at | killer cell lectin-like | Klra3 /// | 16634 /// | −2.05 | 0.00 | 1.09 |
| | receptor, subfamily A, | Klra9 | 16640 | | | |
| | member 3 /// killer cell | | | | | |
| | lectin-like receptor | | | | | |
| | subfamily A, member 9 | | | | | |
| 1449036_PM_at | ring finger protein 128 | Rnf128 | 66889 | 1.54 | 0.00 | 1.69 |
| 1455818_PM_at | RIKEN cDNA 4930427A07 gene | 4930427A07Rik | 104732 | −1.59 | 0.00 | 0.22 |

100MMF-vs-Veh, 12 h

| | | | | | | |
|---|---|---|---|---|---|---|
| 1445666_PM_at | — | — | — | 2.14 | 0.00 | 0.01 |
| 1453068_PM_at | PR domain containing 2, | Prdm2 | 110593 | 1.70 | 0.00 | 0.98 |
| | with ZNF domain | | | | | |

100DMF-vs-90MMF, 7 h

None

100DMF-vs-90MMF, 12 h

None

BRAIN

No differentially expressed genes found in any comparison

CEREBELLUM
100DMF-vs-Veh, 7 h
None
100DMF-vs-Veh, 12 h

None

100MMF-vs-Veh, 7 h

| | | | | | | |
|---|---|---|---|---|---|---|
| 1446570_PM_at | — | — | — | 1.51 | 0.00 | 2.63 |

100MMF-vs-Veh, 12 h

| | | | | | | |
|---|---|---|---|---|---|---|
| 1437136_PM_at | RIKEN cDNA 5830436I19 | 5830436I19Rik | 319946 | 1.87 | 0.00 | 1.31 |
| | gene | | | | | |

100DMF-vs-90MMF, 7 h

None

100DMF-vs-90MMF, 12 h

None

SPINAL CORD
100DMF-vs-Veh, 7 h
Not done
100DMF-vs-Veh, 12 h

| | | | | | | |
|---|---|---|---|---|---|---|
| 1451386_PM_at | biliverdin reductase B (flavin | Blvrb | 233016 | 1.84 | 0.00 | 2.24 |
| | reductase (NADPH)) | | | | | |

100MMF-vs-Veh, 7 h

Not done

100MMF-vs-Veh, 12 h

| | | | | | | |
|---|---|---|---|---|---|---|
| 1445562_PM_at | — | — | — | 2.11 | 0.00 | 1.00 |

100DMF-vs-90MMF, 7 h

Not done

100DMF-vs-90MMF, 12 h

None

SPLEEN
100DMF-vs-Veh, 7 h

| | | | | | | |
|---|---|---|---|---|---|---|
| 1416430_PM_at | catalase | Cat | 12359 | 1.63 | 0.00 | 0.68 |
| 1416632_PM_at | similar to NADP-dependent | LOC677317 /// | 17436 /// | 1.52 | 0.00 | 1.04 |
| | malic enzyme (NADP-ME) | Me1 | 677317 | | | |
| | (Malic enzyme 1) /// malic | | | | | |
| | enzyme 1, NADP(+)- | | | | | |
| | dependent, cytosolic | | | | | |
| 1419435_PM_at | aldehyde oxidase 1 | Aox1 | 11761 | 1.74 | 0.00 | 0.31 |
| 1419942_PM_at | Sulfiredoxin 1 homolog | Srxn1 | 76650 | 2.08 | 0.00 | 21.50 |
| | (S. cerevisiae) | | | | | |
| 1423436_PM_at | glutathione S-transferase, | Gsta3 | 14859 | 1.74 | 0.00 | 0.77 |
| | alpha 3 | | | | | |
| 1423437_PM_at | glutathione S-transferase, | Gsta3 | 14859 | 1.55 | 0.00 | 0.75 |
| | alpha 3 | | | | | |
| 1423627_PM_at | NAD(P)H dehydrogenase, | Nqo1 | 18104 | 3.34 | 0.00 | 8.34 |
| | quinone 1 | | | | | |
| 1425035_PM_s_at | DNA (cytosine-5-)- | Dnmt31 | 54427 | 1.81 | 0.00 | 3.96 |
| | methyltransferase 3-like | | | | | |
| 1425958_PM_at | interleukin 1 family, member 9 | Il1f9 | 215257 | 2.33 | 0.00 | 1.99 |
| 1426261_PM_s_at | UDP glucuronosyltransferase | Ugt1a1 /// | 22236 /// | 1.51 | 0.00 | 0.29 |
| | 1 family, polypeptide A1 /// | Ugt1a10 /// | 394430 /// | | | |
| | UDP glycosyltransferase 1 | Ugt1a2 /// | 394432 /// | | | |
| | family, polypeptide A10 /// | Ugt1a5 /// | 394433 /// | | | |
| | UDP glucuronosyltransferase | Ugt1a6a /// | 394434 /// | | | |
| | 1 family, polypeptide A2 /// | Ugt1a6b /// | 394435 /// | | | |
| | UDP glucuronosyltransferase | Ugt1a7c /// | 394436 /// | | | |
| | 1 family, polypeptide A5 /// | Ugt1a9 | 94284 | | | |
| | UDP glucuronosyltransferase | | | | | |
| | 1 family, polypeptide A6A /// | | | | | |
| | UDP glucuronosyltransferase | | | | | |
| | 1 family, polypeptide A6B /// | | | | | |
| | UDP glucuronosyltransferase | | | | | |
| | 1 family, polypeptide A7C /// | | | | | |
| | UDP glucuronosyltransferase | | | | | |
| | 1 family, polypeptide A9 | | | | | |
| 1426875_PM_s_at | sulfiredoxin 1 homolog | Srxn1 | 76650 | 2.08 | 0.00 | 24.87 |
| | (S. cerevisiae) | | | | | |
| 1428586_PM_at | transmembrane protein 41B | Tmem41b | 233724 | 1.61 | 0.00 | 0.11 |
| 1429001_PM_at | pirin | Pir | 69656 | 2.99 | 0.00 | 2.25 |
| 1434150_PM_a_at | HIG1 domain family, member | Higd1c /// | 380975 /// | 2.00 | 0.00 | 1.59 |
| | 1C /// methyltransferase like | Mettl7a1 /// | 393082 /// | | | |
| | 7A1 /// methyltransferase like | Mettl7a2 | 70152 | | | |
| | 7A2 | | | | | |
| 1435975_PM_at | DENN/MADD domain | Dennd4a | 102442 | 1.56 | 0.00 | 1.36 |
| | containing 4A | | | | | |
| 1437716_PM_x_at | kinesin family member 22 | Kif22 | 110033 | 1.53 | 0.00 | 0.70 |
| 1439050_PM_at | glutamate-cysteine ligase, | Gclm | 14630 | 2.02 | 0.00 | 1.81 |
| | modifier subunit | | | | | |
| 1441413_PM_at | — | — | — | 1.52 | 0.00 | 0.41 |
| 1447837_PM_x_at | polymerase (DNA directed), | Polh | 80905 | 1.51 | 0.00 | 2.18 |
| | eta (RAD 30 related) | | | | | |
| 1451680_PM_at | sulfiredoxin 1 homolog | Srxn1 | 76650 | 1.84 | 0.00 | 8.82 |
| | (S. cerevisiae) | | | | | |
| 1458902_PM_at | — | — | — | 1.69 | 0.00 | 0.14 |

100DMF-vs-Veh, 12 h

Not done

100MMF-vs-Veh, 7 h

| | | | | | | |
|---|---|---|---|---|---|---|
| 1415908_PM_at | testis-specific protein, | Tspyl1 | 22110 | 1.66 | 0.00 | 7.57 |
| | Y-encoded-like 1 | | | | | |
| 1416179_PM_a_at | radixin | Rdx | 19684 | 1.53 | 0.00 | 5.08 |
| 1416429_PM_a_at | catalase | Cat | 12359 | 1.51 | 0.00 | 3.97 |
| 1416499_PM_a_at | dynactin 6 | Dctn6 | 22428 | 1.53 | 0.00 | 3.62 |
| 1416886_PM_at | C1D nuclear receptor co- | C1d | 57316 | 1.52 | 0.00 | 1.08 |
| | repressor | | | | | |
| 1417770_PM_s_at | proteasome (prosome, | Psmc6 | 67089 | 1.77 | 0.00 | 1.48 |
| | macropain) 26S subunit, | | | | | |
| | ATPase, 6 | | | | | |
| 1418070_PM_at | chromodomain protein, Y | Cdyl | 12593 | 1.73 | 0.00 | 1.00 |
| | chromosome-like | | | | | |
| 1419565_PM_a_at | zinc finger protein X-linked | Zfx | 22764 | 1.50 | 0.00 | 4.01 |
| 1419942_PM_at | Sulfiredoxin 1 homolog | Srxn1 | 76650 | 1.82 | 0.00 | 14.95 |
| | (S. cerevisiae) | | | | | |
| 1420042_PM_at | THO complex 1 | Thoc1 | 225160 | 1.89 | 0.00 | 5.06 |
| 1420249_PM_s_at | chemokine (C-C motif) ligand 6 | Ccl6 | 20305 | 1.57 | 0.00 | 0.39 |
| 1422573_PM_at | adenosine monophosphate | Ampd3 | 11717 | 1.61 | 0.00 | 0.94 |
| | deaminase 3 | | | | | |
| 1422621_PM_at | RAN binding protein 2 | Ranbp2 | 19386 | 1.50 | 0.00 | 8.00 |
| 1422716_PM_a_at | acid phosphatase 1, soluble | Acp1 | 11431 | 1.80 | 0.00 | 2.36 |
| 1423627_PM_at | NAD(P)H dehydrogenase, | Nqo1 | 18104 | 3.23 | 0.00 | 7.35 |
| | quinone 1 | | | | | |
| 1424020_PM_at | ADP-ribosylation factor-like 6 | Arl6ip6 | 65103 | 1.52 | 0.00 | 1.96 |
| | interacting protein 6 | | | | | |
| 1424518_PM_at | apolipoprotein L 9a /// | Apol9a /// | 223672 /// | 1.57 | 0.00 | 0.62 |
| | apolipoprotein L 9b | Apol9b | 71898 | | | |
| 1424523_PM_at | engulfment and cell motility 1, | Elmo1 | 140580 | 1.52 | 0.00 | 0.81 |
| | ced-12 homolog (C. elegans) | | | | | |
| 1424596_PM_s_at | LIM and cysteine-rich domains 1 | Lmcd1 | 30937 | 1.64 | 0.00 | 9.04 |
| 1425350_PM_a_at | myelin basic protein expression | Myef2 | 17876 | 1.59 | 0.00 | 5.27 |
| | factor 2, repressor | | | | | |
| 1425514_PM_at | phosphatidylinositol 3-kinase, | Pik3r1 | 18708 | 1.62 | 0.00 | 0.12 |
| | regulatory subunit, polypeptide | | | | | |
| | 1 (p85 alpha) | | | | | |
| 1426875_PM_s_at | sulfiredoxin 1 homolog | Srxn1 | 76650 | 1.94 | 0.00 | 21.10 |
| | (S. cerevisiae) | | | | | |
| 1427275_PM_at | structural maintenance of | Smc4 | 70099 | 1.56 | 0.00 | 2.11 |
| | chromosomes 4 | | | | | |
| 1427318_PM_s_at | myoferlin | Myof | 226101 | 1.52 | 0.00 | 5.06 |
| 1427705_PM_a_at | nuclear factor of kappa light | Nfkb1 | 18033 | 1.52 | 0.00 | 6.30 |
| | polypeptide gene enhancer in | | | | | |
| | B-cells 1, p105 | | | | | |
| 1428091_PM_at | kelch-like 7 (Drosophila) | Klhl7 | 52323 | 1.77 | 0.00 | 0.50 |
| 1428369_PM_s_at | Rho GTPase activating protein 21 | Arhgap21 | 71435 | 1.57 | 0.00 | 2.00 |
| 1428586_PM_at | transmembrane protein 41B | Tmem41b | 233724 | 1.88 | 0.00 | 4.26 |
| 1429001_PM_at | pirin | Pir | 69656 | 2.78 | 0.00 | 1.00 |
| 1429050_PM_at | cysteine-rich hydrophobic | Chic2 | 74277 | 1.67 | 0.00 | 6.36 |
| | domain 2 | | | | | |
| 1429146_PM_at | small VCP/p97-interacting | Svip | 75744 | 1.59 | 0.00 | 0.52 |
| | protein | | | | | |
| 1429436_PM_at | PRP40 pre-mRNA processing | Prpf40a | 56194 | 1.65 | 0.00 | 5.33 |
| | factor 40 homolog A (yeast) | | | | | |
| 1429490_PM_at | Rap1 interacting factor 1 | Rif1 | 51869 | 1.66 | 0.00 | 0.18 |
| | homolog (yeast) | | | | | |
| 1429530_PM_a_at | sphingomyelin | Smpd4 | 77626 | 2.04 | 0.00 | 0.33 |
| | phosphodiesterase 4 | | | | | |
| 1433735_PM_a_at | transmembrane protein 64 | Tmem64 | 100201 | 1.70 | 0.00 | 3.02 |
| 1433934_PM_at | Sec24 related gene family, | Sec24a | 77371 | 1.50 | 0.00 | 3.12 |
| | member A (S. cerevisiae) | | | | | |
| 1434067_PM_at | expressed sequence AI662270 | AI662270 | 100043636 | 1.52 | 0.00 | 2.50 |
| 1434120_PM_a_at | methionine aminopeptidase 2 | Metap2 | 56307 | 1.52 | 0.00 | 0.47 |
| 1434150_PM_a_at | HIG1 domain family, member | Higd1c /// | 380975 /// | 2.40 | 0.00 | 5.23 |
| | 1C /// methyltransferase like | Mettl7a1 /// | 393082 /// | | | |
| | 7A1 /// methyltransferase like | Mettl7a2 | 70152 | | | |
| | 7A2 | | | | | |
| 1434664_PM_at | RIKEN cDNA 2410129H14 | 2410129H14Rik | 76789 | 1.74 | 0.00 | 0.44 |
| | gene | | | | | |
| 1434767_PM_at | expressed sequence C79407 | C79407 | 217653 | 1.95 | 0.00 | 1.44 |
| 1434853_PM_x_at | makorin, ring finger protein, 1 | Mkrn1 | 54484 | 1.62 | 0.00 | 2.79 |
| 1434866_PM_x_at | carnitine palmitoyltransferase | Cpt1a | 12894 | 1.54 | 0.00 | 4.47 |
| | 1a, liver | | | | | |
| 1435092_PM_at | ADP-ribosylation factor-like 4A | Arl4a | 11861 | 2.26 | 0.00 | 1.83 |
| 1435235_PM_at | thioredoxin-like 1 | Txnl1 | 53382 | 1.59 | 0.00 | 2.63 |
| 1435884_PM_at | intersectin 1 (SH3 domain | Itsn1 | 16443 | 1.69 | 0.00 | 0.34 |
| | protein 1A) | | | | | |
| 1435975_PM_at | DENN/MADD domain | Dennd4a | 102442 | 1.71 | 0.00 | 4.07 |
| | containing 4A | | | | | |
| 1436708_PM_x_at | minichromosome maintenance | Mcm4 | 17217 | 1.72 | 0.00 | 1.29 |
| | deficient 4 homolog | | | | | |
| | (S. cerevisiae) | | | | | |
| 1436737_PM_a_at | sorbin and SH3 domain | Sorbs1 | 20411 | 1.71 | 0.00 | 1.28 |
| | containing 1 | | | | | |
| 1437172_PM_x_at | hydroxyacyl-Coenzyme A | Hadhb | 231086 | 1.51 | 0.00 | 5.12 |
| | dehydrogenase/3-ketoacyl- | | | | | |
| | Coenzyme A thiolase/enoyl- | | | | | |
| | Coenzyme A hydratase | | | | | |
| | (trifunctional protein), beta | | | | | |
| | subunit | | | | | |
| 1437363_PM_at | homer homolog 1 (Drosophila) | Homer1 | 26556 | 1.60 | 0.00 | 1.48 |
| 1437508_PM_at | trans-acting transcription factor 4 | Sp4 | 20688 | 1.67 | 0.00 | 3.43 |
| 1437526_PM_x_at | predicted gene 6159 /// | Gm6159 /// | 620521 /// | 1.50 | 0.00 | 4.62 |
| | heterogeneous nuclear | Hnrnpr | 74326 | | | |
| | ribonucleoprotein R | | | | | |
| 1437714_PM_x_at | Ubiquitin specific peptidase 14 | Usp14 | 59025 | 1.57 | 0.00 | 3.12 |
| 1437716_PM_x_at | kinesin family member 22 | Kif22 | 110033 | 1.68 | 0.00 | 3.34 |
| 1437878_PM_s_at | tetratricopeptide repeat domain 14 | Ttc14 | 67120 | 1.53 | 0.00 | 7.37 |
| 1438156_PM_x_at | carnitine palmitoyltransferase | Cpt1a | 12894 | 1.52 | 0.00 | 4.16 |
| | 1a, liver | | | | | |
| 1438259_PM_at | — | — | — | 1.56 | 0.00 | 6.87 |
| 1438292_PM_x_at | adenosine kinase | Adk | 11534 | 1.54 | 0.00 | 1.46 |
| 1438506_PM_s_at | abl-interactor 1 | Abi1 | 11308 | 1.61 | 0.00 | 7.71 |
| 1438511_PM_a_at | RIKEN cDNA 1190002H23 | 1190002H23Rik | 66214 | 1.60 | 0.00 | 3.55 |
| | gene | | | | | |
| 1438786_PM_a_at | RIKEN cDNA 2610021A01 | 2610021A01Rik | 668572 | 1.55 | 0.00 | 1.46 |
| | gene | | | | | |
| 1438931_PM_s_at | similar to Sesn1 protein /// | LOC100047324 /// | 100047324 /// | 1.52 | 0.00 | 0.63 |
| | sestrin 1 | Sesn1 | 140742 | | | |
| 1438985_PM_x_at | OTU domain containing 5 | Otud5 | 54644 | 1.51 | 0.00 | 1.18 |
| 1439012_PM_a_at | deoxycytidine kinase | Dck | 13178 | 1.59 | 0.00 | 2.42 |
| 1439050_PM_at | glutamate-cysteine ligase, | Gclm | 14630 | 1.99 | 0.00 | 1.21 |
| | modifier subunit | | | | | |
| 1439403_PM_x_at | ring finger protein, LIM | Rlim | 19820 | 1.51 | 0.00 | 9.35 |
| | domain interacting | | | | | |
| 1439424_PM_x_at | HERPUD family member 2 | Herpud2 | 80517 | 1.62 | 0.00 | 10.21 |
| 1440132_PM_s_at | protein kinase, cAMP | Prkar1b | 19085 | 1.50 | 0.00 | 1.80 |
| | dependent regulatory, type I | | | | | |
| | beta | | | | | |
| 1441864_PM_x_at | centromere protein A | Cenpa | 12615 | 1.86 | 0.00 | 0.02 |
| 1442745_PM_x_at | RNA binding motif protein 39 | Rbm39 | 170791 | 1.54 | 0.00 | 4.31 |
| 1442959_PM_at | baculoviral IAP repeat- | Birc6 | 12211 | 1.54 | 0.00 | 1.12 |
| | containing 6 | | | | | |
| 1443364_PM_at | — | — | — | 1.88 | 0.00 | 2.29 |
| 1443527_PM_at | telomeric repeat binding factor 1 | Terf1 | 21749 | 1.71 | 0.00 | 5.32 |
| 1443911_PM_at | — | — | — | 1.54 | 0.00 | 7.48 |
| 1444212_PM_at | — | — | — | 1.54 | 0.00 | 14.60 |
| 1445883_PM_at | RAN binding protein 2 | Ranbp2 | 19386 | 1.61 | 0.00 | 6.75 |
| 1446234_PM_at | — | — | — | 1.50 | 0.00 | 6.41 |
| 1446425_PM_at | RIKEN cDNA 4732418C07 | 4732418C07Rik | 230648 | 1.53 | 0.00 | 3.02 |
| | gene | | | | | |
| 1447100_PM_s_at | RIKEN cDNA 5730508B09 | 5730508B09Rik | 70617 | 1.91 | 0.00 | 1.57 |
| | gene | | | | | |
| 1447522_PM_s_at | tankyrase, TRF1-interacting | Tnks2 | 74493 | 1.63 | 0.00 | 3.92 |
| | ankyrin-related ADP-ribose | | | | | |
| | polymerase 2 | | | | | |
| 1447670_PM_at | proteasome (prosome, | Psmd9 | 67151 | 1.73 | 0.00 | 3.55 |
| | macropain) 26S subunit, non- | | | | | |
| | ATPase, 9 | | | | | |
| 1447706_PM_at | — | — | — | 1.57 | 0.00 | 3.72 |
| 1447720_PM_x_at | protein kinase, cAMP | Prkaca | 18747 | 1.53 | 0.00 | 6.12 |
| | dependent, catalytic, alpha | | | | | |
| 1447776_PM_x_at | RAB6, member RAS oncogene | Rab6 | 19346 | 1.51 | 0.00 | 4.76 |
| | family | | | | | |
| 1447837_PM_x_at | polymerase (DNA directed), eta | Polh | 80905 | 1.64 | 0.00 | 4.98 |
| | (RAD 30 related) | | | | | |
| 1449176_PM_a_at | deoxycytidine kinase | Dck | 13178 | 1.89 | 0.00 | 0.60 |
| 1449661_PM_at | Suppressor of zeste 12 homolog | Suz12 | 52615 | 1.56 | 0.00 | 6.17 |
| | (Drosophila) | | | | | |
| 1449699_PM_s_at | RIKEN cDNA C330027C09 | C330027C09Rik | 224171 | 1.59 | 0.00 | 1.35 |
| | gene | | | | | |
| 1450744_PM_at | elongation factor RNA | Ell2 | 192657 | 1.58 | 0.00 | 0.60 |
| | polymerase II 2 | | | | | |
| 1451626_PM_x_at | — | — | — | 1.60 | 0.00 | 4.69 |
| 1451680_PM_at | sulfiredoxin 1 homolog | Srxn1 | 76650 | 1.68 | 0.00 | 5.07 |
| | (S. cerevisiae) | | | | | |
| 1451971_PM_at | cullin 4A | Cul4a | 99375 | 1.71 | 0.00 | 0.53 |
| 1452426_PM_x_at | — | — | — | 1.58 | 0.00 | 0.47 |
| 1452593_PM_a_at | transcription elongation factor | Tceb1 | 67923 | 1.50 | 0.00 | 2.03 |
| | B (SIII), polypeptide 1 | | | | | |
| 1453035_PM_at | limb and neural patterns | Lnp | 69605 | 1.69 | 0.00 | 2.26 |
| 1453139_PM_at | nudix (nucleoside diphosphate | Nudt12 | 67993 | 2.17 | 0.00 | 0.10 |
| | linked moiety X)-type motif 12 | | | | | |
| 1454842_PM_a_at | UDP-GalNAc:betaGlcNAc beta | B3galnt2 | 97884 | 1.51 | 0.00 | 0.47 |
| | 1,3-galactosaminyltransferase, | | | | | |
| | polypeptide 2 | | | | | |
| 1454858_PM_x_at | methyltransferase like 7A1 | Mettl7a1 | 70152 | 1.63 | 0.00 | 0.24 |
| 1454952_PM_s_at | non-SMC condensin II | Ncapd3 | 78658 | 1.66 | 0.00 | 1.24 |
| | complex, subunit D3 | | | | | |
| 1455052_PM_a_at | RIKEN cDNA 2410129H14 | 2410129H14Rik | 76789 | 1.56 | 0.00 | 5.45 |
| | gene | | | | | |
| 1455384_PM_x_at | RIKEN cDNA D030056L22 | D030056L22Rik | 225995 | 1.60 | 0.00 | 1.15 |
| | gene | | | | | |
| 1455489_PM_at | leucine rich repeat | Lrrtm2 | 107065 | 1.50 | 0.00 | 5.78 |
| | transmembrane neuronal 2 | | | | | |
| 1455816_PM_a_at | potassium channel | Kctd3 | 226823 | 1.51 | 0.00 | 5.11 |
| | tetramerisation domain | | | | | |
| | containing 3 | | | | | |
| 1455938_PM_x_at | RAD21 homolog (S. pombe) | Rad21 | 19357 | 1.58 | 0.00 | 6.99 |
| 1456036_PM_x_at | glutathione S-transferase omega 1 | Gsto1 | 14873 | 1.51 | 0.00 | 3.83 |
| 1456071_PM_a_at | cytochrome c, somatic /// | Cycs /// | 13063 /// | 1.64 | 0.00 | 0.14 |
| | predicted gene 10053 | Gm10053 | 672195 | | | |
| 1456510_PM_x_at | HIG1 domain family, member | Higd1c /// | 380975 /// | 1.61 | 0.00 | 1.13 |
| | 1C /// methyltransferase like | Mettl7a2 | 393082 | | | |
| | 7A2 | | | | | |
| 1456728_PM_x_at | aconitase 1 | Aco1 | 11428 | 1.69 | 0.00 | 10.67 |
| 1456790_PM_at | zinc finger protein 800 | Zfp800 | 627049 | 1.83 | 0.00 | 1.59 |
| 1458296_PM_at | — | — | — | 1.79 | 0.00 | 7.97 |
| 1458586_PM_at | — | — | — | 1.50 | 0.00 | 2.05 |
| 1459783_PM_s_at | cappuccino | Cno | 117197 | 1.58 | 0.00 | 6.68 |
| 1460069_PM_at | structural maintenance of | Smc6 | 67241 | 1.52 | 0.00 | 6.47 |
| | chromosomes 6 | | | | | |

100MMF-vs-Veh, 12 h

Not done

100DMF-vs-90MMF, 7 h

None

100DMF-vs-90MMF, 12 h

Not done

LYMPH NODE
100DMF-vs-Veh, 7 h

| | | | | | | |
|---|---|---|---|---|---|---|
| 1417408_PM_at | coagulation factor III | F3 | 14066 | 1.59 | 0.00 | 0.43 |
| 1419435_PM_at | aldehyde oxidase 1 | Aox1 | 11761 | 2.16 | 0.00 | 1.11 |
| 1419942_PM_at | Sulfiredoxin 1 homolog | Srxn1 | 76650 | 1.71 | 0.00 | 7.58 |
| | (S. cerevisiae) | | | | | |
| 1423627_PM_at | NAD(P)H dehydrogenase, | Nqo1 | 18104 | 2.19 | 0.00 | 13.39 |
| | quinone 1 | | | | | |
| 1426875_PM_s_at | sulfiredoxin 1 homolog | Srxn1 | 76650 | 1.68 | 0.00 | 8.46 |
| | (S. cerevisiae) | | | | | |
| 1429001_PM_at | pirin | Pir | 69656 | 1.52 | 0.00 | 1.11 |
| 1439050_PM_at | glutamate-cysteine ligase, | Gclm | 14630 | 1.53 | 0.00 | 5.43 |
| | modifier subunit | | | | | |

100DMF-vs-Veh, 12 h

| | | | | | | |
|---|---|---|---|---|---|---|
| 1419435_PM_at | aldehyde oxidase 1 | Aox1 | 11761 | 1.69 | 0.00 | 0.18 |
| 1423627_PM_at | NAD(P)H dehydrogenase, | Nqo1 | 18104 | 1.69 | 0.00 | 2.89 |
| | quinone 1 | | | | | |

100MMF-vs-Veh, 7 h

| | | | | | | |
|---|---|---|---|---|---|---|
| 1417408_PM_at | coagulation factor III | F3 | 14066 | 1.57 | 0.00 | 0.00 |
| 1419435_PM_at | aldehyde oxidase 1 | Aox1 | 11761 | 2.17 | 0.00 | 1.15 |
| 1419942_PM_at | Sulfiredoxin 1 homolog | Srxn1 | 76650 | 1.65 | 0.00 | 6.06 |
| | (S. cerevisiae) | | | | | |
| 1423627_PM_at | NAD(P)H dehydrogenase, | Nqo1 | 18104 | 1.98 | 0.00 | 9.73 |
| | quinone 1 | | | | | |
| 1426875_PM_s_at | sulfiredoxin 1 homolog | Srxn1 | 76650 | 1.61 | 0.00 | 6.46 |
| | (S. cerevisiae) | | | | | |
| 1439050_PM_at | glutamate-cysteine ligase, | Gclm | 14630 | 1.50 | 0.00 | 4.51 |
| | modifier subunit | | | | | |

100MMF-vs-Veh, 12 h

| | | | | | | |
|---|---|---|---|---|---|---|
| 1437136_PM_at | RIKEN cDNA 5830436I19 gene | 5830436I19Rik | 319946 | 1.65 | 0.00 | 0.38 |
| 1446567_PM_at | — | — | — | 1.74 | 0.00 | 2.91 |
| 1454406_PM_at | RIKEN cDNA 4930453J04 gene | 4930453J04Rik | 74652 | 1.76 | 0.00 | 1.44 |
| 1457549_PM_at | — | — | — | 1.67 | 0.00 | 2.07 |

100DMF-vs-90MMF, 7 h

None

100DMF-vs-90MMF, 12 h

None

### APPENDIX E

### EAE Multidose Gene Lists

| | | | | | |
|---|---|---|---|---|---|
| | Gene | Entrez | | p. | |
| Gene Title | Symbol | Gene | FC | value | lods |

WHOLE BLOOD
100DMF-vs-Veh, 7 h

| | | | | | | |
|---|---|---|---|---|---|---|
| 1416125_PM_at | FK506 binding protein 5 | Fkbp5 | 14229 | 1.91 | 0.00 | 0.60 |
| 1416316_PM_at | solute carrier family 27 (fatty | Slc27a2 | 26458 | −1.73 | 0.00 | 1.37 |
| | acid transporter), member 2 | | | | | |
| 1419942_PM_at | Sulfiredoxin 1 homolog | Srxn1 | 76650 | 1.74 | 0.00 | 1.41 |
| | (S. cerevisiae) | | | | | |
| 1420984_PM_at | phosphatidylcholine transfer | Pctp | 18559 | 1.51 | 0.00 | 4.29 |
| | protein | | | | | |
| 1426875_PM_s_at | sulfiredoxin 1 homolog | Srxn1 | 76650 | 1.66 | 0.00 | 0.69 |
| | (S. cerevisiae) | | | | | |
| 1434470_PM_at | synaptotagmin XIII | Syt13 | 80976 | 2.25 | 0.00 | 3.69 |
| 1443673_PM_x_at | — | — | — | 2.20 | 0.00 | 8.18 |
| 1449036_PM_at | ring finger protein 128 | Rnf128 | 66889 | 3.07 | 0.00 | 22.90 |

100DMF-vs-Veh, 12 h

| | | | | | | |
|---|---|---|---|---|---|---|
| 1424631_PM_a_at | Immunoglobulin heavy chain | Ighg | 380794 | −4.01 | 0.00 | 3.03 |
| | (gamma polypeptide) | | | | | |
| 1424975_PM_at | sialic acid binding Ig-like lectin 5 | Siglec5 | 233186 | −2.53 | 0.00 | 0.53 |
| 1428789_PM_at | Ral GEF with PH domain and | Ralgps2 | 78255 | −1.52 | 0.00 | 0.14 |
| | SH3 binding motif 2 | | | | | |
| 1429786_PM_a_at | ZW10 interactor | Zwint | 52696 | 1.87 | 0.00 | 2.72 |
| 1435574_PM_at | — | — | — | −1.55 | 0.00 | 1.59 |
| 1449036_PM_at | ring finger protein 128 | Rnf128 | 66889 | 1.90 | 0.00 | 8.83 |
| 1451680_PM_at | sulfiredoxin 1 homolog | Srxn1 | 76650 | 1.70 | 0.00 | 0.49 |
| | (S. cerevisiae) | | | | | |
| 1451814_PM_a_at | HIV-1 tat interactive protein 2, | Htatip2 | 53415 | 1.80 | 0.00 | 0.36 |
| | homolog (human) | | | | | |

100MMF-vs-Veh, 7 h

| | | | | | | |
|---|---|---|---|---|---|---|
| 1419942_PM_at | Sulfiredoxin 1 homolog | Srxn1 | 76650 | 1.79 | 0.00 | 0.95 |
| | (S. cerevisiae) | | | | | |
| 1421137_PM_a_at | protein kinase inhibitor beta, | Pkib | 18768 | −1.65 | 0.00 | 1.39 |
| | cAMP dependent, testis specific | | | | | |
| 1425351_PM_at | sulfiredoxin 1 homolog | Srxn1 | 76650 | 1.68 | 0.00 | 0.88 |
| | (S. cerevisiae) | | | | | |
| 1425829_PM_a_at | STEAP family member 4 | Steap4 | 117167 | −2.62 | 0.00 | 1.27 |
| 1426875_PM_s_at | sulfiredoxin 1 homolog | Srxn1 | 76650 | 1.72 | 0.00 | 0.59 |
| | (S. cerevisiae) | | | | | |
| 1428976_PM_at | thymopoietin | Tmpo | 21917 | −1.51 | 0.00 | 1.42 |
| 1430373_PM_at | RIKEN cDNA 5430427O19 gene | 5430427O19Rik | 71398 | −1.65 | 0.00 | 2.40 |
| 1434470_PM_at | synaptotagmin XIII | Syt13 | 80976 | 2.11 | 0.00 | 1.11 |
| 1437190_PM_at | serine/threonine/tyrosine kinase 1 | Styk1 | 243659 | 1.60 | 0.00 | 1.90 |
| 1438716_PM_at | expressed sequence AI451617 | AI451617 | 209387 | −1.72 | 0.00 | 0.82 |
| 1443071_PM_at | expressed sequence AI839979 | AI839979 | 100740 | −1.66 | 0.00 | 0.04 |
| 1447339_PM_at | — | — | — | 1.76 | 0.00 | 0.43 |
| 1448385_PM_at | solute carrier family 15, member 4 | Slc15a4 | 100561 | −1.59 | 0.00 | 6.49 |
| 1448613_PM_at | extracellular matrix protein 1 | Ecm1 | 13601 | 1.60 | 0.00 | 1.58 |
| 1449036_PM_at | ring finger protein 128 | Rnf128 | 66889 | 2.71 | 0.00 | 17.11 |
| 1449851_PM_at | period homolog 1 (Drosophila) | Per1 | 18626 | 1.86 | 0.00 | 2.72 |
| 1451680_PM_at | sulfiredoxin 1 homolog | Srxn1 | 76650 | 1.84 | 0.00 | 1.79 |
| | (S. cerevisiae) | | | | | |
| 1456494_PM_a_at | expressed sequence AI451617 /// | AI451617 /// | 20128 /// | −1.76 | 0.00 | 0.42 |
| | tripartite motif-containing 30 | Trim30 | 209387 | | | |
| 1457976_PM_at | RIKEN cDNA 2010002M12 gene | 2010002M12Rik | 112419 | −2.17 | 0.00 | 0.25 |

100MMF-vs-Veh, 12 h

| | | | | | | |
|---|---|---|---|---|---|---|
| 1417953_PM_at | family with sequence similarity 3, | Fam3c | 27999 | −1.50 | 0.00 | 1.19 |
| | member C | | | | | |
| 1418117_PM_at | NADH dehydrogenase | Ndufs4 | 17993 | −1.57 | 0.00 | 2.31 |
| | (ubiquinone) Fe—S protein 4 | | | | | |
| 1424631_PM_a_at | Immunoglobulin heavy chain | Ighg | 380794 | −3.14 | 0.00 | 0.17 |
| | (gamma polypeptide) | | | | | |
| 1429786_PM_a_at | ZW10 interactor | Zwint | 52696 | 1.90 | 0.00 | 2.88 |
| 1449036_PM_at | ring finger protein 128 | Rnf128 | 66889 | 1.59 | 0.00 | 2.74 |
| 1451174_PM_at | leucine rich repeat containing 33 | Lrrc33 | 224109 | −1.51 | 0.00 | 0.08 |
| 1456803_PM_at | Polymerase (RNA) III (DNA | Polr3c | 74414 | 1.89 | 0.00 | 0.26 |
| | directed) polypeptide C | | | | | |

100DMF-vs-90MMF, 7 h

None

100DMF-vs-90MMF, 12 h

None

BRAIN
100DMF-vs-Veh, 7 h

| | | | | | | |
|---|---|---|---|---|---|---|
| 1419435_PM_at | aldehyde oxidase 1 | Aox1 | 11761 | 1.67 | 0.00 | 23.53 |
| 1419606_PM_a_at | troponin T1, skeletal, slow | Tnnt1 | 21955 | 1.54 | 0.00 | 1.24 |
| 1425408_PM_a_at | RIKEN cDNA 2610034M16 gene | 2610034M16Rik | 69239 | −1.53 | 0.00 | 1.64 |
| 1441429_PM_at | insulin receptor substrate 4 | Irs4 | 16370 | −2.25 | 0.00 | 0.61 |
| 1447694_PM_x_at | neogenin | Neo1 | 18007 | 2.30 | 0.00 | 0.41 |
| 1460668_PM_at | galanin | Gal | 14419 | −1.83 | 0.00 | 0.60 |

100DMF-vs-Veh, 12 h

None

100MMF-vs-Veh, 7 h

| | | | | | | |
|---|---|---|---|---|---|---|
| 1415908_PM_at | testis-specific protein, | Tspyl1 | 22110 | −1.57 | 0.00 | 10.77 |
| | Y-encoded-like 1 | | | | | |
| 1416499_PM_a_at | dynactin 6 | Dctn6 | 22428 | −1.55 | 0.00 | 7.98 |
| 1416530_PM_a_at | similar to purine nucleoside | LOC100045567 /// | 100045567 /// | −1.75 | 0.00 | 3.21 |
| | phosphorylase /// | Pnp | 18950 | | | |
| | purine-nucleoside | | | | | |
| | phosphorylase | | | | | |
| 1416711_PM_at | T-box brain gene 1 | Tbr1 | 21375 | −1.69 | 0.00 | 12.66 |
| 1416862_PM_at | signal transducing adaptor | Stam | 20844 | −1.53 | 0.00 | 5.01 |
| | molecule (SH3 domain and | | | | | |
| | ITAM motif) 1 | | | | | |
| 1417188_PM_s_at | ubiquitin-conjugating | Ube2k | 53323 | −1.53 | 0.00 | 4.10 |
| | enzyme E2K (UBC1 homolog, yeast) | | | | | |
| 1418585_PM_at | cyclin H | Ccnh | 66671 | −1.62 | 0.00 | 12.68 |
| 1418690_PM_at | protein tyrosine phosphatase, | Ptprz1 | 19283 | −1.51 | 0.00 | 7.20 |
| | receptor type Z, | | | | | |
| | polypeptide 1 | | | | | |
| 1419381_PM_at | telomeric repeat binding factor 2, | Terf2ip | 57321 | −1.60 | 0.00 | 10.70 |
| | interacting protein | | | | | |
| 1419435_PM_at | aldehyde oxidase 1 | Aox1 | 11761 | 1.67 | 0.00 | 22.92 |
| 1419565_PM_a_at | zinc finger protein X-linked | Zfx | 22764 | −1.69 | 0.00 | 6.92 |
| 1419591_PM_at | gasdermin C | Gsdmc | 83492 | 1.63 | 0.00 | 7.67 |
| 1419606_PM_a_at | troponin T1, skeletal, slow | Tnnt1 | 21955 | 1.63 | 0.00 | 2.78 |
| 1420017_PM_at | tetraspanin 8 | Tspan8 | 216350 | 2.59 | 0.00 | 10.58 |
| 1420042_PM_at | THO complex 1 | Thoc1 | 225160 | −1.67 | 0.00 | 10.12 |
| 1420868_PM_s_at | transmembrane emp24 domain | Tmed2 | 56334 | −1.64 | 0.00 | 3.42 |
| | trafficking protein 2 | | | | | |
| 1421269_PM_at | UDP-glucose ceramide | Ugcg | 22234 | −1.60 | 0.00 | 2.22 |
| | glucosyltransferase | | | | | |
| 1422313_PM_a_at | insulin-like growth factor | Igfbp5 | 16011 | −1.54 | 0.00 | 5.23 |
| | binding protein 5 | | | | | |
| 1422450_PM_at | catenin (cadherin associated | Ctnnd1 | 12388 | −1.62 | 0.00 | 5.68 |
| | protein), delta 1 | | | | | |
| 1422966_PM_a_at | transferrin receptor | Tfrc | 22042 | −1.62 | 0.00 | 2.55 |
| 1423176_PM_at | transducer of ErbB-2.1 | Tob1 | 22057 | −1.52 | 0.00 | 5.38 |
| 1423627_PM_at | NAD(P)H dehydrogenase, | Nqo1 | 18104 | 1.50 | 0.00 | 17.16 |
| | quinone 1 | | | | | |
| 1423747_PM_a_at | pyruvate dehydrogenase kinase, | Pdk1 | 228026 | −1.53 | 0.00 | 7.63 |
| | isoenzyme 1 | | | | | |
| 1423851_PM_a_at | shisa homolog 2 | Shisa2 | 219134 | −1.55 | 0.00 | 3.86 |
| | (Xenopus laevis) | | | | | |
| 1425350_PM_a_at | myelin basic protein expression | Myef2 | 17876 | −1.50 | 0.00 | 12.19 |
| | factor 2, repressor | | | | | |
| 1425485_PM_at | myotubularin related protein 6 | Mtmr6 | 219135 | −1.58 | 0.00 | 13.58 |
| 1425495_PM_at | zinc finger protein 62 | Zfp62 | 22720 | −1.64 | 0.00 | 10.51 |
| 1425537_PM_at | protein phosphatase 1A, | Ppm1a | 19042 | −1.52 | 0.00 | 5.59 |
| | magnesium dependent, alpha | | | | | |
| | isoform | | | | | |
| 1426060_PM_at | — | — | — | −1.67 | 0.00 | 12.57 |
| 1426061_PM_x_at | — | — | — | −1.50 | 0.00 | 8.27 |
| 1426104_PM_at | mitogen-activated protein kinase 14 | Mapk14 | 26416 | 1.57 | 0.00 | 6.14 |
| 1426394_PM_at | eukaryotic translation initiation | Eif3j | 78655 | −1.53 | 0.00 | 12.43 |
| | factor 3, subunit J | | | | | |
| 1426556_PM_at | zinc finger protein 280D | Zfp280d | 235469 | −1.51 | 0.00 | 8.82 |
| 1426583_PM_at | activating transcription factor 2 | Atf2 | 11909 | −1.79 | 0.00 | 7.95 |
| 1427067_PM_at | RIKEN cDNA 4933439F18 gene | 4933439F18Rik | 66771 | −1.52 | 0.00 | 2.87 |
| 1427122_PM_at | coatomer protein complex, subunit | Copg2as2 | 100044236 | −1.66 | 0.00 | 11.06 |
| | gamma 2, antisense 2 | | | | | |
| 1427157_PM_at | coiled-coil domain containing 85A | Ccdc85a | 216613 | −1.51 | 0.00 | 2.05 |
| 1428091_PM_at | kelch-like 7 (Drosophila) | Klhl7 | 52323 | −1.63 | 0.00 | 8.75 |
| 1428210_PM_s_at | conserved helix-loop-helix | Chuk | 12675 | −1.51 | 0.00 | 6.91 |
| | ubiquitous kinase | | | | | |
| 1428352_PM_at | arrestin domain containing 2 | Arrdc2 | 70807 | 1.54 | 0.00 | 5.67 |
| 1428586_PM_at | transmembrane protein 41B | Tmem41b | 233724 | −1.94 | 0.00 | 7.59 |
| 1429211_PM_at | cell adhesion molecule 2 | Cadm2 | 239857 | −1.65 | 0.00 | 10.25 |
| 1429371_PM_at | zinc ringer protein 788 | Zfp788 | 67607 | −1.70 | 0.00 | 12.29 |
| 1429417_PM_at | chondroitin sulfate synthase 3 | Chsy3 | 78923 | −1.51 | 0.00 | 4.34 |
| 1429430_PM_at | protein-L-isoaspartate | Pcmtd1 | 319263 | −1.58 | 0.00 | 20.80 |
| | (D-aspartate) O-methyltransferase | | | | | |
| | domain containing 1 | | | | | |
| 1429490_PM_at | Rap1 interacting factor 1 homolog | Rif1 | 51869 | −1.52 | 0.00 | 1.95 |
| | (yeast) | | | | | |
| 1429712_PM_at | predicted gene 14288 | Gm14288 | 13999 | −1.85 | 0.00 | 8.36 |
| 1429978_PM_at | antagonist of mitotic exit | Amn1 | 232566 | −1.63 | 0.00 | 6.41 |
| | network 1 homolog | | | | | |
| | (S. cerevisiae) | | | | | |
| 1433047_PM_at | RIKEN cDNA 5330430B06 gene | 5330430B06Rik | 78280 | 1.50 | 0.00 | 6.09 |
| 1433735_PM_a_at | transmembrane protein 64 | Tmem64 | 100201 | −1.74 | 0.00 | 5.57 |
| 1433837_PM_at | RIKEN cDNA 8430408G22 gene | 8430408G22Rik | 213393 | 2.03 | 0.00 | 0.08 |
| 1433856_PM_at | diphosphoinositol | Ppip5k2 | 227399 | −1.50 | 0.00 | 9.63 |
| | pentakisphosphate kinase 2 | | | | | |
| 1433914_PM_at | expressed sequence AI747699 | AI747699 | 381236 | −2.07 | 0.00 | 9.31 |
| 1434150_PM_a_at | HIG1 domain family, member | Higd1c /// | 380975 /// | −1.52 | 0.00 | 7.65 |
| | 1C /// methyltransferase | Mettl7a1 /// | 393082 /// | | | |
| | like 7A1 /// methyltransferase | Mettl7a2 | 70152 | | | |
| | like 7A2 | | | | | |
| 1434236_PM_at | zinc finger, DHHC domain | Zdhhc20 | 75965 | −1.64 | 0.00 | 6.71 |
| | containing 20 | | | | | |
| 1434405_PM_at | folliculin interacting protein 1 | Fnip1 | 216742 | −1.60 | 0.00 | 7.94 |
| 1434475_PM_at | peptidyl-prolyl isomerase G | Ppig | 228005 | −1.53 | 0.00 | 2.09 |
| | (cyclophilin G) | | | | | |
| 1434819_PM_at | beta galactoside alpha 2,6 | St6gal2 | 240119 | −1.53 | 0.00 | 6.81 |
| | sialyltransferase 2 | | | | | |
| 1434860_PM_at | dpy-19-like 4 (C. elegans) | Dpy19l4 | 381510 | −1.60 | 0.00 | 10.10 |
| 1435146_PM_s_at | cell adhesion molecule 2 | Cadm2 | 239857 | −1.87 | 0.00 | 8.80 |
| 1435164_PM_s_at | ubiquitin-like modifier | Uba3 | 22200 | −1.53 | 0.00 | 9.49 |
| | activating enzyme 3 | | | | | |
| 1435284_PM_at | reticulon 4 | Rtn4 | 68585 | −1.52 | 0.00 | 6.33 |
| 1435435_PM_at | cortactin binding protein 2 | Cttnbp2 | 30785 | −1.67 | 0.00 | 7.67 |
| 1435514_PM_at | leucine zipper transcription | Lztfl1 | 93730 | −1.65 | 0.00 | 7.89 |
| | factor-like 1 | | | | | |
| 1435597_PM_at | ATPase family, AAA domain | Atad5 | 237877 | −1.52 | 0.00 | 12.41 |
| | containing 5 | | | | | |
| 1435640_PM_x_at | RIKEN cDNA A130040M12 gene | A130040M12Rik | 319269 | −1.57 | 0.00 | 0.11 |
| 1435814_PM_at | exportin 7 | Xpo7 | 65246 | −1.61 | 0.00 | 11.51 |
| 1435822_PM_at | RIKEN cDNA D830012I24 gene | D830012I24Rik | 320070 | −1.51 | 0.00 | 22.36 |
| 1436116_PM_x_at | adaptor protein, phosphotyrosine | Appl1 | 72993 | −1.58 | 0.00 | 1.98 |
| | interaction, PH domain and | | | | | |
| | leucine zipper containing 1 | | | | | |
| 1436139_PM_at | — | — | — | −1.80 | 0.00 | 10.21 |
| 1436708_PM_x_at | minichromosome maintenance | Mcm4 | 17217 | −1.59 | 0.00 | 6.75 |
| | deficient 4 homolog | | | | | |
| | (S. cerevisiae) | | | | | |
| 1436761_PM_s_at | family with sequence | Fam13c | 71721 | −1.53 | 0.00 | 6.23 |
| | similarity 13, member C | | | | | |
| 1436944_PM_x_at | phosphatidylserine decarboxylase, | Pisd-ps1 /// | 236604 /// | −1.82 | 0.00 | 8.12 |
| | pseudogene 1 /// | Pisd-ps3 | 66776 | | | |
| | phosphatidylserine decarboxylase, | | | | | |
| | pseudogene 3 | | | | | |
| 1436946_PM_s_at | predicted gene 13342 /// | Gm13342 /// | 100041120 /// | −1.63 | 0.00 | 9.47 |
| | predicted gene 15776 /// | Gm15776 /// | 100041703 /// | | | |
| | predicted gene 3150 /// guanine | Gm3150 /// | 100043507 /// | | | |
| | nucleotide binding protein | Gng5 /// | 100044719 /// | | | |
| | (G protein), gamma 5 /// | LOC100044719 | 14707 | | | |
| | similar to G protein gamma-5 | | | | | |
| | subunit | | | | | |
| 1437147_PM_at | gamma-aminobutyric acid | Gabrg2 | 14406 | −1.56 | 0.00 | 8.90 |
| | (GABA) A receptor, subunit gamma 2 | | | | | |
| 1437152_PM_at | mex3 homolog B (C. elegans) | Mex3b | 108797 | −1.64 | 0.00 | 13.88 |
| 1437168_PM_at | splicing factor, arginine/serine- | Sfrs13b | 272009 | −1.72 | 0.00 | 13.57 |
| | rich 13B | | | | | |
| 1437172_PM_x_at | hydroxyacyl-Coenzyme A | Hadhb | 231086 | −1.63 | 0.00 | 10.89 |
| | dehydrogenase/3-ketoacyl- | | | | | |
| | Coenzyme A thiolase/enoyl- | | | | | |
| | Coenzyme A hydratase | | | | | |
| | (trifunctional protein), beta | | | | | |
| | subunit | | | | | |
| 1437200_PM_at | FCH domain only 2 | Fcho2 | 218503 | −1.70 | 0.00 | 11.65 |
| 1437508_PM_at | trans-acting transcription | Sp4 | 20688 | −1.60 | 0.00 | 12.88 |
| | factor 4 | | | | | |
| 1437671_PM_x_at | protease, serine, 23 | Prss23 | 76453 | −1.73 | 0.00 | 1.06 |
| 1437837_PM_x_at | polymerase (DNA-directed), | Poldip3 | 73826 | −1.55 | 0.00 | 5.94 |
| | delta interacting protein 3 | | | | | |
| 1437878_PM_s_at | tetratricopeptide repeat domain 14 | Ttc14 | 67120 | −1.84 | 0.00 | 9.87 |
| 1438259_PM_at | — | — | — | −1.53 | 0.00 | 5.16 |
| 1438506_PM_s_at | abl-interactor 1 | Abi1 | 11308 | −1.72 | 0.00 | 10.93 |
| 1438553_PM_x_at | RIKEN cDNA 4930453N24 gene | 4930453N24Rik | 67609 | −1.51 | 0.00 | 14.84 |
| 1438562_PM_a_at | protein tyrosine phosphatase, non- | Ptpn2 | 19255 | −1.82 | 0.00 | 10.70 |
| | receptor type 2 | | | | | |
| 1438786_PM_a_at | RIKEN cDNA 2610021A01 gene | 2610021A01Rik | 668572 | −1.60 | 0.00 | 4.36 |
| 1439249_PM_at | WW domain containing adaptor with | Wac | 225131 | −1.53 | 0.00 | 12.82 |
| | coiled-coil | | | | | |
| 1439424_PM_x_at | HERPUD family member 2 | Herpud2 | 80517 | −1.63 | 0.00 | 12.81 |
| 1439446_PM_at | cDNA sequence BC048507 | BC048507 | 408058 | 1.56 | 0.00 | 2.73 |
| 1439618_PM_at | phosphodiesterase 10A | Pde10a | 23984 | −1.52 | 0.00 | 2.17 |
| 1439619_PM_at | transcription factor 12 | Tcf12 | 21406 | −1.51 | 0.00 | 2.35 |
| 1439906_PM_at | — | — | — | −1.51 | 0.00 | 5.48 |
| 1440162_PM_x_at | hypothetical protein A630043P06 | A630043P06 | 328187 | 1.71 | 0.00 | 8.69 |
| 1440516_PM_at | SLIT and NTRK-like family, | Slitrk4 | 245446 | −1.54 | 0.00 | 7.50 |
| | member 4 | | | | | |
| 1441228_PM_at | apolipoprotein L domain | Apold1 | 381823 | 1.59 | 0.00 | 2.27 |
| | containing 1 | | | | | |
| 1441550_PM_at | RIKEN cDNA 9330184L24 gene | 9330184L24Rik | 402729 | −1.51 | 0.00 | 7.56 |
| 1441791_PM_at | — | — | — | −1.93 | 0.00 | 6.61 |
| 1441799_PM_at | RIKEN cDNA 6030422H21 gene | 6030422H21Rik | 402765 | 1.76 | 0.00 | 1.66 |
| 1441890_PM_x_at | transmembrane protein with EGF- | Tmeff1 | 230157 | −1.58 | 0.00 | 11.47 |
| | like and two follistatin-like | | | | | |
| | domains 1 | | | | | |
| 1441942_PM_x_at | snurportin 1 | Snupn | 66069 | 1.59 | 0.00 | 1.00 |
| 1442029_PM_at | KCNQ1 overlapping transcript 1 | Kcnq1ot1 | 63830 | −1.52 | 0.00 | 1.60 |
| 1442786_PM_s_at | RUN and FYVE domain containing 3 | Rufy3 | 52822 | −1.54 | 0.00 | 9.31 |
| 1443364_PM_at | — | — | — | −1.70 | 0.00 | 5.48 |
| 1445642_PM_at | LEM domain containing 1 | Lemd1 | 213409 | −1.54 | 0.00 | 4.12 |
| 1446425_PM_at | RIKEN cDNA 4732418C07 gene | 4732418C07Rik | 230648 | −1.75 | 0.00 | 9.42 |
| 1446622_PM_at | RIKEN cDNA A330068G13 gene | A330068G13Rik | 414087 | −1.56 | 0.00 | 5.79 |
| 1446642_PM_at | PHD finger protein 14 | Phf14 | 75725 | −1.55 | 0.00 | 5.97 |
| 1447522_PM_s_at | tankyrase, TRF1-interacting | Tnks2 | 74493 | −1.53 | 0.00 | 7.75 |
| | ankyrin-related | | | | | |
| | ADP-ribose polymerase 2 | | | | | |
| 1447670_PM_at | proteasome (prosome, macropain) 26S | Psmd9 | 67151 | −1.53 | 0.00 | 2.19 |
| | subunit, non-ATPase, 9 | | | | | |
| 1447694_PM_x_at | neogenin | Neo1 | 18007 | 6.12 | 0.00 | 16.60 |
| 1447706_PM_at | — | — | — | −1.58 | 0.00 | 1.19 |
| 1447726_PM_at | ripply2 homolog (zebrafish) | Ripply2 | 382089 | −1.61 | 0.00 | 4.78 |
| 1447776_PM_x_at | RAB6, member RAS oncogene family | Rab6 | 19346 | −1.61 | 0.00 | 6.08 |
| 1447813_PM_x_at | src-like adaptor | Sla | 20491 | 1.95 | 0.00 | 8.52 |
| 1447882_PM_x_at | DEAD (Asp-Glu-Ala-Asp) box | Ddx54 | 71990 | 1.69 | 0.00 | 0.99 |
| | polypeptide 54 | | | | | |
| 1448002_PM_x_at | RIKEN cDNA 2610001J05 gene | 2610001J05Rik | 66520 | 6.51 | 0.00 | 11.04 |
| 1449176_PM_a_at | deoxycytidine kinase | Dck | 13178 | −1.77 | 0.00 | 4.43 |
| 1449357_PM_at | RIKEN cDNA 2310030G06 gene | 2310030G06Rik | 66952 | 1.62 | 0.00 | 3.55 |
| 1449661_PM_at | Suppressor of zeste 12 homolog | Suz12 | 52615 | −1.56 | 0.00 | 13.79 |
| | (Drosophila) | | | | | |
| 1449851_PM_at | period homolog 1 (Drosophila) | Per1 | 18626 | 1.57 | 0.00 | 13.49 |
| 1449913_PM_at | zinc finger protein 2 | Zfp2 | 22678 | −1.51 | 0.00 | 9.32 |
| 1449972_PM_s_at | cDNA sequence BC018101 /// zinc | BC018101 /// | 22759 /// | −1.54 | 0.00 | 9.07 |
| | finger protein 97 | Zfp97 | 449000 | | | |
| 1450484_PM_a_at | cytidine monophosphate (UMP-CMP) | Cmpk2 | 22169 | −1.54 | 0.00 | 7.62 |
| | kinase 2, mitochondrial | | | | | |
| 1450896_PM_at | Rho GTPase activating protein 5 | Arhgap5 | 11855 | −1.53 | 0.00 | 4.38 |
| 1451612_PM_at | metallothionein 1 | Mt1 | 17748 | −1.53 | 0.00 | 3.21 |
| 1451790_PM_a_at | tissue factor pathway inhibitor | Tfpi | 21788 | −1.57 | 0.00 | 2.42 |
| 1452007_PM_at | vesicle-associated membrane protein 7 | Vamp7 | 20955 | −1.68 | 0.00 | 12.19 |
| 1452090_PM_a_at | olfactomedin 3 | Olfm3 | 229759 | −1.54 | 0.00 | 10.06 |
| 1452758_PM_s_at | eukaryotic translation initiation | Eif4g2 | 13690 | −1.68 | 0.00 | 6.23 |
| | factor 4, gamma 2 | | | | | |
| 1453035_PM_at | limb and neural patterns | Lnp | 69605 | −1.63 | 0.00 | 13.32 |
| 1453245_PM_at | RIKEN cDNA 9130024F11 gene | 9130024F11Rik | 329160 | −1.54 | 0.00 | 3.66 |
| 1453807_PM_at | RIKEN cDNA 6330563C09 gene | 6330563C09Rik | 76186 | −1.54 | 0.00 | 1.27 |
| 1454604_PM_s_at | tetraspanin 12 | Tspan12 | 269831 | −1.54 | 0.00 | 10.53 |
| 1454725_PM_at | transformer 2 alpha homolog | Tra2a | 101214 | −1.57 | 0.00 | 10.69 |
| | (Drosophila) | | | | | |
| 1455603_PM_at | — | — | — | −1.50 | 0.00 | 12.46 |
| 1455816_PM_a_at | potassium channel tetramerisation | Kctd3 | 226823 | −1.51 | 0.00 | 4.36 |
| | domain containing 3 | | | | | |
| 1455882_PM_x_at | von Willebrand factor C domain | Vwc2 | 319922 | −1.69 | 0.00 | 1.17 |
| | containing 2 | | | | | |
| 1455908_PM_a_at | serine carboxypeptidase 1 | Scpep1 | 74617 | −1.56 | 0.00 | 5.75 |
| 1455928_PM_x_at | leucine-zipper-like | Lztr1 | 66863 | −1.57 | 0.00 | 11.49 |
| | transcriptional regulator, 1 | | | | | |
| 1455978_PM_a_at | matrilin 2 | Matn2 | 17181 | −2.04 | 0.00 | 9.74 |
| 1455995_PM_at | DNA segment, Chr 10, Brigham | D10Bwg1379e | 215821 | −1.64 | 0.00 | 14.51 |
| | & Women's Genetics 1379 expressed | | | | | |
| 1456041_PM_at | sorting nexin 16 | Snx16 | 74718 | −1.76 | 0.00 | 6.60 |
| 1456071_PM_a_at | cytochrome c, somatic /// | Cycs /// | 13063 /// | −1.63 | 0.00 | 11.94 |
| | predicted gene 10053 | Gm10053 | 672195 | | | |
| 1456089_PM_at | tripartite motif-containing 23 | Trim23 | 81003 | −1.56 | 0.00 | 14.07 |
| 1456219_PM_at | similar to OPR /// zinc finger | LOC100045988 /// | 100045988 /// | −1.56 | 0.00 | 2.15 |
| | protein of the cerebellum 5 | Zic5 | 65100 | | | |
| 1456509_PM_at | RIKEN cDNA 1110032F04 gene | 1110032F04Rik | 68725 | −1.58 | 0.00 | 11.84 |
| 1456728_PM_x_at | aconitase 1 | Aco1 | 11428 | −1.52 | 0.00 | 7.68 |
| 1456901_PM_at | a disintegrin-like and | Adamts20 | 223838 | −1.52 | 0.00 | 7.91 |
| | metallopeptidase (reprolysin type) | | | | | |
| | with thrombospondin | | | | | |
| | type 1 motif, 20 | | | | | |
| 1456917_PM_at | ADP-ribosylation factor guanine | Arfgef1 | 211673 | −1.69 | 0.00 | 5.89 |
| | nucleotide-exchange factor | | | | | |
| | 1(brefeldin A-inhibited) | | | | | |
| 1457832_PM_at | — | — | — | −1.69 | 0.00 | 19.62 |
| 1458296_PM_at | — | — | — | −1.55 | 0.00 | 4.50 |
| 1458586_PM_at | — | — | — | −1.54 | 0.00 | 6.59 |
| 1459557_PM_at | — | — | — | 2.14 | 0.00 | 5.21 |
| 1459749_PM_s_at | FAT tumor suppressor homolog 4 | Fat4 | 329628 | −1.83 | 0.00 | 4.70 |
| | (Drosophila) | | | | | |
| 1459971_PM_at | potassium channel, subfamily T, | Kcnt2 | 240776 | −1.56 | 0.00 | 6.95 |
| | member 2 | | | | | |
| 1460004_PM_x_at | syntaxin 6 | Stx6 | 58244 | −1.59 | 0.00 | 2.10 |

100MMF-vs-Veh, 12 h

None

100DMF-vs-90MMF, 7 h

| | | | | | | |
|---|---|---|---|---|---|---|
| 1420017_PM_at | tetraspanin 8 | Tspan8 | 216350 | −1.86 | 0.00 | 1.97 |
| 1420868_PM_s_at | transmembrane emp24 domain | Tmed2 | 56334 | 1.54 | 0.00 | 1.41 |
| | trafficking protein 2 | | | | | |
| 1426060_PM_at | — | — | — | 1.51 | 0.00 | 7.39 |
| 1426583_PM_at | activating transcription | Atf2 | 11909 | 1.62 | 0.00 | 4.01 |
| | factor 2 | | | | | |
| 1427682_PM_a_at | early growth response 2 | Egr2 | 13654 | −1.80 | 0.00 | 0.83 |
| 1428586_PM_at | transmembrane protein 41B | Tmem41b | 233724 | 1.60 | 0.00 | 1.33 |
| 1429211_PM_at | cell adhesion molecule 2 | Cadm2 | 239857 | 1.51 | 0.00 | 5.98 |
| 1432838_PM_at | — | — | — | 1.53 | 0.00 | 0.22 |
| 1433914_PM_at | expressed sequence AI747699 | AI747699 | 381236 | 1.91 | 0.00 | 6.68 |
| 1434236_PM_at | zinc finger, DHHC domain | Zdhhc20 | 75965 | 1.54 | 0.00 | 4.23 |
| | containing 20 | | | | | |
| 1435146_PM_s_at | cell adhesion molecule 2 | Cadm2 | 239857 | 1.64 | 0.00 | 4.03 |
| 1435435_PM_at | cortactin binding protein 2 | Cttnbp2 | 30785 | 1.52 | 0.00 | 3.73 |
| 1435514_PM_at | leucine zipper transcription | Lztfl1 | 93730 | 1.53 | 0.00 | 4.60 |
| | factor-like 1 | | | | | |
| 1436139_PM_at | — | — | — | 1.52 | 0.00 | 3.16 |
| 1437200_PM_at | FCH domain only 2 | Fcho2 | 218503 | 1.51 | 0.00 | 5.59 |
| 1437878_PM_s_at | tetratricopeptide repeat | Ttc14 | 67120 | 1.72 | 0.00 | 7.13 |
| | domain 14 | | | | | |
| 1438562_PM_a_at | protein tyrosine phosphatase, | Ptpn2 | 19255 | 1.51 | 0.00 | 2.96 |
| | non-receptor type 2 | | | | | |
| 1439618_PM_at | phosphodiesterase 10A | Pde10a | 23984 | 1.51 | 0.00 | 1.95 |
| 1441228_PM_at | apolipoprotein L domain | Apold1 | 381823 | −1.55 | 0.00 | 1.38 |
| | containing 1 | | | | | |
| 1441606_PM_at | — | — | — | 1.60 | 0.00 | 4.66 |
| 1442701_PM_at | — | — | — | 1.59 | 0.00 | 2.47 |
| 1443364_PM_at | — | — | — | 1.62 | 0.00 | 3.85 |
| 1445306_PM_at | — | — | — | 1.68 | 0.00 | 1.82 |
| 1446425_PM_at | RIKEN cDNA 4732418C07 gene | 4732418C07Rik | 230648 | 1.54 | 0.00 | 4.05 |
| 1446537_PM_at | — | — | — | 1.51 | 0.00 | 2.56 |
| 1447694_PM_x_at | neogenin | Neo1 | 18007 | −2.66 | 0.00 | 2.50 |
| 1447813_PM_x_at | src-like adaptor | Sla | 20491 | −1.79 | 0.00 | 5.59 |
| 1452758_PM_s_at | eukaryotic translation | Eif4g2 | 13690 | 1.53 | 0.00 | 2.44 |
| | initiation factor 4, gamma 2 | | | | | |
| 1453807_PM_at | RIKEN cDNA | 6330563C09Rik | 76186 | 1.64 | 0.00 | 3.36 |
| | 6330563C09 gene | | | | | |
| 1456917_PM_at | ADP-ribosylation factor guanine | Arfgef1 | 211673 | 1.54 | 0.00 | 2.26 |
| | nucleotide-exchange factor | | | | | |
| | 1(brefeldin A-inhibited) | | | | | |
| 1458663_PM_at | — | — | — | 1.53 | 0.00 | 7.11 |

100DMF-vs-90MMF, 12 h

None

CEREBELLUM
100DMF-vs-Veh, 7 h
None
100DMF-vs-Veh, 12 h

None

100MMF-vs-Veh, 7 h

| | | Gene | Entrez | | p. | |
|---|---|---|---|---|---|---|
| Probe Set ID | Gene Title | Symbol | Gene | FC | value | lods |
| 1435172_PM_at | eomesodermin homolog | Eomes | 13813 | 1.51 | 0.00 | 0.44 |
| | (Xenopus laevis) | | | | | |
| 1459557_PM_at | — | — | — | 2.30 | 0.00 | 1.14 |

100MMF-vs-Veh, 12 h

| | | Gene | Entrez | | p. | |
|---|---|---|---|---|---|---|
| | Gene Title | Symbol | Gene | FC | value | lods |
| 1442025_PM_a_at | — | — | — | −1.84 | 0.00 | 0.19 |
| 1442026_PM_at | — | — | — | −1.91 | 0.00 | 0.09 |
| 1454223_PM_at | RIKEN cDNA 4933423P22 gene | 4933423P22Rik | 71158 | 1.53 | 0.00 | 1.48 |

100DMF-vs-90MMF, 7 h

| | | Gene | Entrez | | p. | |
|---|---|---|---|---|---|---|
| Probe Set ID | Gene Title | Symbol | Gene | FC | value | lods |
| 1455034_PM_at | nuclear receptor subfamily 4, group A, | Nr4a2 | 18227 | −1.51 | 0.00 | 0.33 |
| | member 2 | | | | | |
| 1459289_PM_at | — | — | — | 1.52 | 0.00 | 4.53 |

| | | | | | |
|---|---|---|---|---|---|
| | Gene | Entrez | | p. | |
| Gene Title | Symbol | Gene | FC | value | lods |

100DMF-vs-90MMF, 12 h

| | | | | | | |
|---|---|---|---|---|---|---|
| 1430172_PM_a_at | cytochrome P450, family 4, subfamily f, | Cyp4f16 /// | 677156 /// | 1.54 | 0.00 | 0.80 |
| | polypeptide 16 /// predicted gene 9705 | Gm9705 | 70101 | | | |
| 1438796_PM_at | nuclear receptor subfamily 4, group A, | Nr4a3 | 18124 | −1.82 | 0.00 | 0.47 |
| | member 3 | | | | | |
| 1444901_PM_at | — | — | — | −1.68 | 0.00 | 3.41 |

SPINAL CORD
100DMF-vs-Veh, 7 h

| | | | | | | |
|---|---|---|---|---|---|---|
| 1418318_PM_at | ring finger protein 128 | Rnf128 | 66889 | 1.72 | 0.00 | 3.59 |
| 1419435_PM_at | aldehyde oxidase 1 | Aox1 | 11761 | 1.68 | 0.00 | 3.35 |
| 1449036_PM_at | ring finger protein 128 | Rnf128 | 66889 | 1.60 | 0.00 | 4.44 |

100DMF-vs-Veh, 12 h

| | | | | | | |
|---|---|---|---|---|---|---|
| 1417898_PM_a_at | granzyme A | Gzma | 14938 | −4.64 | 0.00 | 0.57 |
| 1423813_PM_at | kinesin family member 22 | Kif22 | 110033 | −1.62 | 0.00 | 1.16 |
| 1446104_PM_at | — | — | — | 1.57 | 0.00 | 1.42 |

100MMF-vs-Veh, 7 h

| | | | | | | |
|---|---|---|---|---|---|---|
| 1419697_PM_at | chemokine (C-X-C motif) ligand 11 | Cxcl11 | 56066 | −3.36 | 0.00 | 0.48 |
| 1423555_PM_a_at | interferon-induced protein 44 | Ifi44 | 99899 | −1.99 | 0.00 | 1.08 |
| 1431406_PM_at | alanine-glyoxylate aminotransferase | Agxt2l1 | 71760 | 1.78 | 0.00 | 3.57 |
| | 2-like 1 | | | | | |
| 1436562_PM_at | DEAD (Asp-Glu-Ala-Asp) box | Ddx58 | 230073 | −1.67 | 0.00 | 0.19 |
| | polypeptide 58 | | | | | |
| 1450454_PM_at | torsin family 3, member A | Tor3a | 30935 | −1.57 | 0.00 | 0.78 |
| 1450783_PM_at | interferon-induced protein with | Ifit1 | 15957 | −1.67 | 0.00 | 0.17 |
| | tetratricopeptide repeats 1 | | | | | |
| 1451335_PM_at | placenta-specific 8 | Plac8 | 231507 | −2.04 | 0.00 | 0.49 |

100MMF-vs-Veh, 12 h

| | | | | | | |
|---|---|---|---|---|---|---|
| 1418174_PM_at | D site albumin promoter binding | Dbp | 13170 | −1.58 | 0.00 | 0.19 |
| | protein | | | | | |
| 1439946_PM_at | — | — | — | −1.62 | 0.00 | 0.05 |

100DMF-vs-90MMF, 7 h

| | | | | | | |
|---|---|---|---|---|---|---|
| 1430675_PM_at | RIKEN cDNA 2900055J20 gene | 2900055J20Rik | 73001 | −1.58 | 0.00 | 3.12 |
| 1446154_PM_at | hypothetical protein | LOC100047123 | 100047123 | −1.56 | 0.00 | 1.18 |
| | LOC100047123 | | | | | |

100DMF-vs-90MMF, 12 h

| | | | | | | |
|---|---|---|---|---|---|---|
| 1417898_PM_a_at | granzyme A | Gzma | 14938 | −5.32 | 0.00 | 0.87 |
| 1418919_PM_at | shugoshin-like 1 (S. pombe) | Sgol1 | 72415 | −1.61 | 0.00 | 1.00 |
| 1421327_PM_at | hypothetical protein | A730034C02 /// | 17756 /// | 1.69 | 0.00 | 3.10 |
| | A730034C02 /// | Mtap2 | 269204 | | | |
| | microtubule-associated | | | | | |
| | protein 2 | | | | | |
| 1423020_PM_at | — | — | — | 1.59 | 0.00 | 0.34 |
| 1423813_PM_at | kinesin family member 22 | Kif22 | 110033 | −1.68 | 0.00 | 1.26 |
| 1427068_PM_x_at | RIKEN cDNA 4933439F18 gene | 4933439F18Rik | 66771 | 1.52 | 0.00 | 1.08 |
| 1432408_PM_a_at | RIKEN cDNA A230006K03 gene | A230006K03Rik | 27493 | 1.59 | 0.00 | 0.52 |
| 1433015_PM_at | RIKEN cDNA 6330436F06 gene | 6330436F06Rik | 76733 | 1.52 | 0.00 | 1.94 |
| 1433187_PM_at | RIKEN cDNA B230112I24 gene | B230112I24Rik | 77984 | 1.64 | 0.00 | 3.25 |
| 1434946_PM_at | DnaJ (Hsp40) homolog, | Dnajc27 | 217378 | 1.85 | 0.00 | 0.61 |
| | subfamily C, member 27 | | | | | |
| 1437559_PM_at | regulator of G-protein signalling | Rgs7bp | 52882 | 1.50 | 0.00 | 0.30 |
| | 7 binding protein | | | | | |
| 1439850_PM_at | cDNA sequence | BC066028 | 407812 | 1.56 | 0.00 | 0.21 |
| | BC066028 | | | | | |
| 1440090_PM_at | Solute carrier family 25, | Slc25a27 | 74011 | 1.56 | 0.00 | 1.30 |
| | member 27 | | | | | |
| 1440425_PM_at | — | — | — | 1.57 | 0.00 | 0.53 |
| 1440449_PM_at | — | — | — | 1.61 | 0.00 | 1.87 |
| 1441606_PM_at | — | — | — | 1.57 | 0.00 | 0.05 |
| 1442243_PM_at | period homolog 3 (Drosophila) | Per3 | 18628 | 1.54 | 0.00 | 1.08 |
| 1442650_PM_at | — | — | — | 1.92 | 0.00 | 0.05 |
| 1442813_PM_at | — | — | — | 2.21 | 0.00 | 2.71 |
| 1443212_PM_at | — | — | — | 1.82 | 0.00 | 1.37 |
| 1443237_PM_at | — | — | — | 1.59 | 0.00 | 0.06 |
| 1443422_PM_at | RIKEN cDNA 2410089E03 gene | 2410089E03Rik | 73692 | 1.55 | 0.00 | 0.06 |
| 1444001_PM_at | — | — | — | 1.58 | 0.00 | 0.28 |
| 1445534_PM_at | Filamin, beta | Flnb | 286940 | 1.55 | 0.00 | 0.21 |
| 1446265_PM_at | dynamin 3 | Dnm3 | 103967 | 1.54 | 0.00 | 1.01 |
| 1446481_PM_at | — | — | — | 1.59 | 0.00 | 1.71 |
| 1446536_PM_at | sema domain, transmembrane domain | Sema6d | 214968 | 1.57 | 0.00 | 2.80 |
| | (TM), and cytoplasmic domain, | | | | | |
| | (semaphorin) 6D | | | | | |
| 1447354_PM_at | — | — | — | 1.54 | 0.00 | 0.42 |
| 1448650_PM_a_at | polymerase (DNA directed), epsilon | Pole | 18973 | −1.60 | 0.00 | 1.85 |
| 1449958_PM_a_at | fibroblast growth factor 14 | Fgf14 | 14169 | 1.76 | 0.00 | 0.63 |
| 1450477_PM_at | 5-hydroxytryptamine (serotonin) | Htr2c | 15560 | 1.65 | 0.00 | 0.54 |
| | receptor 2C | | | | | |
| 1453956_PM_a_at | cyclin-dependent kinase 14 | Cdk14 | 18647 | 1.76 | 0.00 | 1.39 |
| 1456639_PM_at | zinc finger protein 398 | Zfp398 | 272347 | 1.57 | 0.00 | 1.61 |
| 1458073_PM_at | RIKEN cDNA 9330156P08 gene | 9330156P08Rik | 320141 | 2.08 | 0.00 | 0.34 |
| 1458147_PM_at | — | — | — | 1.60 | 0.00 | 0.38 |
| 1458493_PM_a_at | RIKEN cDNA 2410089E03 gene | 2410089E03Rik | 73692 | 2.00 | 0.00 | 1.08 |
| 1458697_PM_at | — | — | — | 1.72 | 0.00 | 1.05 |
| 1459235_PM_at | — | — | — | 1.65 | 0.00 | 0.65 |
| 1459288_PM_at | — | — | — | 1.66 | 0.00 | 0.80 |
| 1459310_PM_at | — | — | — | 1.87 | 0.00 | 1.05 |

SPLEEN
100DMF-vs-Veh, 7 h

| | | | | | | |
|---|---|---|---|---|---|---|
| 1416632_PM_at | similar to NADP-dependent malic enzyme | LOC677317 /// | 17436 /// | 1.61 | 0.00 | 7.33 |
| | (NADP-ME) (Malic enzyme 1) /// malic | Me1 | 677317 | | | |
| | enzyme 1, NADP(+)-dependent, cytosolic | | | | | |
| 1419435_PM_at | aldehyde oxidase 1 | Aox1 | 11761 | 1.88 | 0.00 | 4.13 |
| 1419942_PM_at | Sulfiredoxin 1 homolog | Srxn1 | 76650 | 2.31 | 0.00 | 29.91 |
| | (S. cerevisiae) | | | | | |
| 1423436_PM_at | glutathione S-transferase, alpha 3 | Gsta3 | 14859 | 2.02 | 0.00 | 10.52 |
| 1423437_PM_at | glutathione S-transferase, alpha 3 | Gsta3 | 14859 | 1.94 | 0.00 | 12.27 |
| 1423627_PM_at | NAD(P)H dehydrogenase, quinone 1 | Nqo1 | 18104 | 2.27 | 0.00 | 6.39 |
| 1425829_PM_a_at | STEAP family member 4 | Steap4 | 117167 | −1.88 | 0.00 | 1.20 |
| 1426875_PM_s_at | sulfiredoxin 1 homolog | Srxn1 | 76650 | 2.29 | 0.00 | 31.88 |
| | (S. cerevisiae) | | | | | |
| 1429001_PM_at | pirin | Pir | 69656 | 2.74 | 0.00 | 2.88 |
| 1451680_PM_at | sulfiredoxin 1 homolog | Srxn1 | 76650 | 2.14 | 0.00 | 23.05 |
| | (S. cerevisiae) | | | | | |
| 1454269_PM_s_at | RIKEN cDNA 4930519L02 gene | 4930519L02Rik | 75085 | 1.66 | 0.00 | 1.85 |
| 1455965_PM_at | a disintegrin-like and | Adamts4 | 240913 | −1.51 | 0.00 | 4.10 |
| | metallopeptidase (reprolysin type) with | | | | | |
| | thrombospondin type 1 motif, 4 | | | | | |

100DMF-vs-Veh, 12 h

| | | | | | | |
|---|---|---|---|---|---|---|
| 1418174_PM_at | D site albumin promoter binding | Dbp | 13170 | −1.65 | 0.00 | 0.17 |
| | protein | | | | | |
| 1419942_PM_at | Sulfiredoxin 1 homolog | Srxn1 | 76650 | 1.85 | 0.00 | 13.91 |
| | (S. cerevisiae) | | | | | |
| 1423627_PM_at | NAD(P)H dehydrogenase, quinone 1 | Nqo1 | 18104 | 1.96 | 0.00 | 2.79 |
| 1424631_PM_a_at | Immunoglobulin heavy chain | Ighg | 380794 | −1.52 | 0.00 | 0.44 |
| | (gamma polypeptide) | | | | | |
| 1426875_PM_s_at | sulfiredoxin 1 homolog | Srxn1 | 76650 | 1.90 | 0.00 | 14.14 |
| | (S. cerevisiae) | | | | | |
| 1438211_PM_s_at | D site albumin promoter binding | Dbp | 13170 | −1.78 | 0.00 | 0.78 |
| | protein | | | | | |
| 1451680_PM_at | sulfiredoxin 1 homolog | Srxn1 | 76650 | 1.60 | 0.00 | 8.73 |
| | (S. cerevisiae) | | | | | |

100MMF-vs-Veh, 7 h

| | | | | | | |
|---|---|---|---|---|---|---|
| 1416576_PM_at | suppressor of cytokine signaling 3 | Socs3 | 12702 | −1.79 | 0.00 | 1.86 |
| 1417273_PM_at | pyruvate dehydrogenase kinase, | Pdk4 | 27273 | 1.68 | 0.00 | 0.30 |
| | isoenzyme 4 | | | | | |
| 1419435_PM_at | aldehyde oxidase 1 | Aox1 | 11761 | 1.91 | 0.00 | 3.83 |
| 1419942_PM_at | Sulfiredoxin 1 homolog | Srxn1 | 76650 | 2.29 | 0.00 | 28.27 |
| | (S. cerevisiae) | | | | | |
| 1423436_PM_at | glutathione S-transferase, alpha 3 | Gsta3 | 14859 | 1.74 | 0.00 | 4.73 |
| 1423437_PM_at | glutathione S-transferase, alpha 3 | Gsta3 | 14859 | 1.61 | 0.00 | 4.19 |
| 1423627_PM_at | NAD(P)H dehydrogenase, quinone 1 | Nqo1 | 18104 | 2.35 | 0.00 | 6.53 |
| 1424486_PM_a_at | thioredoxin reductase 1 | Txnrd1 | 50493 | 1.69 | 0.00 | 1.01 |
| 1424596_PM_s_at | LIM and cysteine-rich domains 1 | Lmcd1 | 30937 | −1.74 | 0.00 | 8.99 |
| 1425829_PM_a_at | STEAP family member 4 | Steap4 | 117167 | −1.91 | 0.00 | 1.08 |
| 1426875_PM_s_at | sulfiredoxin 1 homolog | Srxn1 | 76650 | 2.26 | 0.00 | 29.86 |
| | (S. cerevisiae) | | | | | |
| 1428866_PM_at | RIKEN cDNA 2810037O22 gene | 2810037O22Rik | 72711 | −1.88 | 0.00 | 3.23 |
| 1429001_PM_at | pirin | Pir | 69656 | 2.67 | 0.00 | 1.92 |
| 1434150_PM_a_at | HIG1 domain family, member | Higd1c /// | 380975 /// | 1.57 | 0.00 | 0.01 |
| | 1C /// methyltransferase like | Mettl7a1 /// | 393082 /// | | | |
| | 7A1 /// methyltransferase like | Mettl7a2 | 70152 | | | |
| | 7A2 | | | | | |
| 1435994_PM_at | potassium voltage-gated channel, | Kcnh1 | 16510 | −1.60 | 0.00 | 5.43 |
| | subfamily H (eag-related), | | | | | |
| | member 1 | | | | | |
| 1437568_PM_at | matrix metallopeptidase 16 | Mmp16 | 17389 | −1.56 | 0.00 | 0.90 |
| 1438953_PM_at | c-fos induced growth factor /// | Figf /// | 100047108 /// | 1.57 | 0.00 | 3.40 |
| | similar to FIGF | LOC100047108 | 14205 | | | |
| 1441413_PM_at | — | — | — | 1.54 | 0.00 | 5.35 |
| 1448656_PM_at | calcium channel, voltage- | Cacnb3 | 12297 | −1.58 | 0.00 | 3.91 |
| | dependent, beta 3 subunit | | | | | |
| 1451626_PM_x_at | — | — | — | 1.55 | 0.00 | 6.65 |
| 1451680_PM_at | sulfiredoxin 1 homo log | Srxn1 | 76650 | 2.05 | 0.00 | 20.00 |
| | (S. cerevisiae) | | | | | |
| 1454269_PM_s_at | RIKEN cDNA 4930519L02 gene | 4930519L02Rik | 75085 | 1.78 | 0.00 | 3.41 |
| 1460197_PM_a_at | STEAP family member 4 | Steap4 | 117167 | −2.48 | 0.00 | 0.59 |

100MMF-vs-Veh, 12 h

| | | | | | | |
|---|---|---|---|---|---|---|
| 1416958_PM_at | nuclear receptor subfamily 1, | Nr1d2 | 353187 | −1.53 | 0.00 | 2.21 |
| | group D, member 2 | | | | | |
| 1418174_PM_at | D site albumin promoter binding | Dbp | 13170 | −1.85 | 0.00 | 3.13 |
| | protein | | | | | |
| 1419942_PM_at | Sulfiredoxin 1 homolog | Srxn1 | 76650 | 1.73 | 0.00 | 10.86 |
| | (S. cerevisiae) | | | | | |
| 1423627_PM_at | NAD(P)H dehydrogenase, quinone 1 | Nqo1 | 18104 | 2.05 | 0.00 | 3.87 |
| 1426464_PM_at | nuclear receptor subfamily 1, | Nr1d1 | 217166 | −1.76 | 0.00 | 5.45 |
| | group D, member 1 | | | | | |
| 1426875_PM_s_at | sulfiredoxin 1 homolog | Srxn1 | 76650 | 1.75 | 0.00 | 10.41 |
| | (S. cerevisiae) | | | | | |
| 1428866_PM_at | RIKEN cDNA 2810037O22 gene | 2810037O22Rik | 72711 | −1.55 | 0.00 | 1.48 |
| 1432097_PM_a_at | DNA cross-link repair 1A, PSO2 | Dclre1a | 55947 | 1.56 | 0.00 | 1.04 |
| | homolog (S. cerevisiae) | | | | | |
| 1438211_PM_s_at | D site albumin promoter binding | Dbp | 13170 | −1.90 | 0.00 | 2.30 |
| | protein | | | | | |

100DMF-vs-90MMF, 7 h

| | | | | | | |
|---|---|---|---|---|---|---|
| 1421802_PM_at | eosinophil-associated, | Ear1 | 13586 | −3.57 | 0.00 | 0.10 |
| | ribonuclease A family, member 1 | | | | | |
| 1422873_PM_at | proteoglycan 2, bone marrow | Prg2 | 19074 | −2.10 | 0.00 | 0.16 |
| 1449136_PM_at | eosinophil peroxidase | Epx | 13861 | −3.20 | 0.00 | 0.22 |

100DMF-vs-90MMF, 12 h

| | | | | | | |
|---|---|---|---|---|---|---|
| 1417130_PM_s_at | angiopoietin-like 4 | Angptl4 | 57875 | 1.97 | 0.00 | 2.28 |
| 1419874_PM_x_at | zinc finger and BTB domain | Zbtb16 | 235320 | 1.95 | 0.00 | 2.19 |
| | containing 16 | | | | | |
| 1442025_PM_a_at | — | Zbtb16 | — | 2.29 | 0.00 | 3.00 |
| 1442026_PM_at | — | Zbtb16 | — | 2.36 | 0.00 | 1.98 |

LYMPH NODE
100DMF-vs-Veh, 7 h

| | | | | | | |
|---|---|---|---|---|---|---|
| 1416273_PM_at | tumor necrosis factor, alpha- | Tnfaip2 | 21928 | 1.82 | 0.00 | 5.32 |
| | induced protein 2 | | | | | |
| 1419942_PM_at | Sulfiredoxin 1 homolog | Srxn1 | 76650 | 1.98 | 0.00 | 15.40 |
| | (S. cerevisiae) | | | | | |
| 1420330_PM_at | C-type lectin domain family 4, | Clec4e | 56619 | 1.55 | 0.00 | 3.11 |
| | member e | | | | | |
| 1420361_PM_at | solute carrier family 11 | Slc11a1 | 18173 | 1.57 | 0.00 | 5.93 |
| | (proton-coupled divalent metal | | | | | |
| | ion transporters), member 1 | | | | | |
| 1420804_PM_s_at | C-type lectin domain family 4, | Clec4d | 17474 | 1.85 | 0.00 | 3.65 |
| | member d | | | | | |
| 1423627_PM_at | NAD(P)H dehydrogenase, | Nqo1 | 18104 | 2.79 | 0.00 | 29.54 |
| | quinone 1 | | | | | |
| 1424022_PM_at | oxidative stress induced | Osgin1 | 71839 | 1.54 | 0.00 | 4.04 |
| | growth inhibitor 1 | | | | | |
| 1424783_PM_a_at | UDP glucuronosyltransferase 1 | Ugt1a1 /// | 22236 /// | 1.52 | 0.00 | 0.35 |
| | family, polypeptide A1 /// | Ugt1a10 /// | 394430 /// | | | |
| | UDP glycosyltransferase 1 | Ugt1a2 /// | 394432 /// | | | |
| | family, polypeptide A10 /// | Ugt1a5 /// | 394433 /// | | | |
| | UDP glucuronosyltransferase 1 | Ugt1a6a /// | 394434 /// | | | |
| | family, polypeptide A2 /// | Ugt1a6b /// | 394435 /// | | | |
| | UDP glucuronosyltransferase 1 | Ugt1a7c /// | 394436 /// | | | |
| | family, polypeptide A5 /// | Ugt1a9 | 94284 | | | |
| | UDP glucuronosyltransferase 1 | | | | | |
| | family, polypeptide A6A /// | | | | | |
| | UDP glucuronosyltransferase 1 | | | | | |
| | family, polypeptide A6B /// | | | | | |
| | UDP glucuronosyltransferase 1 | | | | | |
| | family, polypeptide A7C /// | | | | | |
| | UDP glucuronosyltransferase 1 | | | | | |
| | family, polypeptide A9 | | | | | |
| 1426261_PM_s_at | UDP glucuronosyltransferase 1 | Ugt1a1 /// | 22236 /// | 1.62 | 0.00 | 3.88 |
| | family, polypeptide A1 /// | Ugt1a10 /// | 394430 /// | | | |
| | UDP glycosyltransferase 1 | Ugt1a2 /// | 394432 /// | | | |
| | family, polypeptide A10 /// | Ugt1a5 /// | 394433 /// | | | |
| | UDP glucuronosyltransferase 1 | Ugt1a6a /// | 394434 /// | | | |
| | family, polypeptide A2 /// | Ugt1a6b /// | 394435 /// | | | |
| | UDP glucuronosyltransferase 1 | Ugt1a7c /// | 394436 /// | | | |
| | family, polypeptide A5 /// | Ugt1a9 | 94284 | | | |
| | UDP glucuronosyltransferase 1 | | | | | |
| | family, polypeptide A6A /// | | | | | |
| | UDP glucuronosyltransferase 1 | | | | | |
| | family, polypeptide A6B /// | | | | | |
| | UDP glucuronosyltransferase 1 | | | | | |
| | family, polypeptide A7C /// | | | | | |
| | UDP glucuronosyltransferase 1 | | | | | |
| | family, polypeptide A9 | | | | | |
| 1426875_PM_s_at | sulfiredoxin 1 homolog | Srxn1 | 76650 | 1.83 | 0.00 | 13.93 |
| | (S. cerevisiae) | | | | | |
| 1438855_PM_x_at | tumor necrosis factor, alpha- | Tnfaip2 | 21928 | 1.52 | 0.00 | 5.90 |
| | induced protein 2 | | | | | |
| 1449036_PM_at | ring finger protein 128 | Rnf128 | 66889 | 1.71 | 0.00 | 3.38 |
| 1449153_PM_at | matrix metallopeptidase 12 | Mmp12 | 17381 | 3.33 | 0.00 | 5.08 |
| 1449288_PM_at | growth differentiation factor 3 | Gdf3 | 14562 | −1.57 | 0.00 | 1.64 |

100DMF-vs-Veh, 12 h

| | | | | | | |
|---|---|---|---|---|---|---|
| 1419874_PM_x_at | zinc finger and BTB domain | Zbtb16 | 235320 | 2.07 | 0.00 | 1.67 |
| | containing 16 | | | | | |
| 1420804_PM_s_at | C-type lectin domain family 4, | Clec4d | 17474 | 2.19 | 0.00 | 3.64 |
| | member d | | | | | |
| 1421037_PM_at | neuronal PAS domain protein 2 | Npas2 | 18143 | 1.53 | 0.00 | 1.41 |
| 1422280_PM_at | granzyme K | Gzmk | 14945 | 1.92 | 0.00 | 0.97 |
| 1423627_PM_at | NAD(P)H dehydrogenase, | Nqo1 | 18104 | 1.70 | 0.00 | 8.54 |
| | quinone 1 | | | | | |
| 1424783_PM_a_at | UDP glucuronosyltransferase 1 | Ugt1a1 /// | 22236 /// | 1.56 | 0.00 | 2.84 |
| | family, polypeptide A1 /// | Ugt1a10 /// | 394430 /// | | | |
| | UDP glycosyltransferase 1 | Ugt1a2 /// | 394432 /// | | | |
| | family, polypeptide A10 /// | Ugt1a5 /// | 394433 /// | | | |
| | UDP glucuronosyltransferase 1 | Ugt1a6a /// | 394434 /// | | | |
| | family, polypeptide A2 /// | Ugt1a6b /// | 394435 /// | | | |
| | UDP glucuronosyltransferase 1 | Ugt1a7c /// | 394436 /// | | | |
| | family, polypeptide A5 /// | Ugt1a9 | 94284 | | | |
| | UDP glucuronosyltransferase 1 | | | | | |
| | family, polypeptide A6A /// | | | | | |
| | UDP glucuronosyltransferase 1 | | | | | |
| | family, polypeptide A6B /// | | | | | |
| | UDP glucuronosyltransferase 1 | | | | | |
| | family, polypeptide A7C /// | | | | | |
| | UDP glucuronosyltransferase 1 | | | | | |
| | family, polypeptide A9 | | | | | |
| 1442025_PM_a_at | — | — | — | 2.35 | 0.00 | 1.52 |
| 1442026_PM_at | — | — | — | 2.48 | 0.00 | 1.92 |

100MMF-vs-Veh, 7 h

| | | | | | | |
|---|---|---|---|---|---|---|
| 1416273_PM_at | tumor necrosis factor, alpha- | Tnfaip2 | 21928 | 1.86 | 0.00 | 4.91 |
| | induced protein 2 | | | | | |
| 1416953_PM_at | connective tissue growth | Ctgf | 14219 | 2.34 | 0.00 | 8.00 |
| | factor | | | | | |
| 1417061_PM_at | solute carrier family 40 (iron- | Slc40a1 | 53945 | 1.80 | 0.00 | 5.78 |
| | regulated transporter), | | | | | |
| | member 1 | | | | | |
| 1419942_PM_at | Sulfiredoxin 1 homolog | Srxn1 | 76650 | 1.93 | 0.00 | 12.74 |
| | (S. cerevisiae) | | | | | |
| 1420330_PM_at | C-type lectin domain family | Clec4e | 56619 | 2.02 | 0.00 | 12.03 |
| | 4, member e | | | | | |
| 1420331_PM_at | C-type lectin domain family | Clec4e | 56619 | 1.80 | 0.00 | 6.48 |
| | 4, member e | | | | | |
| 1420361_PM_at | solute carrier family 11 | Sic11a1 | 18173 | 1.57 | 0.00 | 4.63 |
| | (proton-coupled divalent | | | | | |
| | metal ion transporters), | | | | | |
| | member 1 | | | | | |
| 1420804_PM_s_at | C-type lectin domain family | Clec4d | 17474 | 2.40 | 0.00 | 9.76 |
| | 4, member d | | | | | |
| 1423450_PM_a_at | heparan sulfate (glucosamine) | Hs3st1 | 15476 | 1.72 | 0.00 | 8.78 |
| | 3-O-sulfotransferase 1 | | | | | |
| 1423627_PM_at | NAD(P)H dehydrogenase, | Nqo1 | 18104 | 2.59 | 0.00 | 24.98 |
| | quinone 1 | | | | | |
| 1424022_PM_at | oxidative stress induced | Osgin1 | 71839 | 1.50 | 0.00 | 2.20 |
| | growth inhibitor 1 | | | | | |
| 1425958_PM_at | interleukin 1 family, member 9 | Il1f9 | 215257 | 1.86 | 0.00 | 2.03 |
| 1426188_PM_s_at | cDNA sequence BC005685 | BC005685 | 352945 | 1.54 | 0.00 | 4.79 |
| 1426261_PM_s_at | UDP glucuronosyltransferase | Ugt1a1 /// | 22236 /// | 1.55 | 0.00 | 1.60 |
| | 1 family, polypeptide A1 /// | Ugt1a10 /// | 394430 /// | | | |
| | UDP glycosyltransferase 1 | Ugt1a2 /// | 394432 /// | | | |
| | family, polypeptide A10 /// | Ugt1a5 /// | 394433 /// | | | |
| | UDP glucuronosyltransferase | Ugt1a6a /// | 394434 /// | | | |
| | 1 family, polypeptide A2 /// | Ugt1a6b /// | 394435 /// | | | |
| | UDP glucuronosyltransferase | Ugt1a7c /// | 394436 /// | | | |
| | 1 family, polypeptide A5 /// | Ugt1a9 | 94284 | | | |
| | UDP glucuronosyltransferase | | | | | |
| | 1 family, polypeptide A6A /// | | | | | |
| | UDP glucuronosyltransferase | | | | | |
| | 1 family, polypeptide A6B /// | | | | | |
| | UDP glucuronosyltransferase | | | | | |
| | 1 family, polypeptide A7C /// | | | | | |
| | UDP glucuronosyltransferase | | | | | |
| | 1 family, polypeptide A9 | | | | | |
| 1426875_PM_s_at | sulfiredoxin 1 homolog | Srxn1 | 76650 | 1.80 | 0.00 | 11.69 |
| | (S. cerevisiae) | | | | | |
| 1430700_PM_a_at | phospholipase A2, group VII | Pla2g7 | 27226 | 1.67 | 0.00 | 1.29 |
| | (platelet-activating factor | | | | | |
| | acetylhydrolase, plasma) | | | | | |
| 1438855_PM_x_at | tumor necrosis factor, alpha- | Tnfaip2 | 21928 | 1.55 | 0.00 | 5.73 |
| | induced protein 2 | | | | | |
| 1442498_PM_at | EST C78662 | C78662 | 30863 | 1.68 | 0.00 | 2.88 |
| 1448566_PM_at | solute carrier family 40 (iron- | Slc40a1 | 53945 | 1.87 | 0.00 | 4.19 |
| | regulated transporter), | | | | | |
| | member 1 | | | | | |
| 1449036_PM_at | ring finger protein 128 | Rnf128 | 66889 | 1.81 | 0.00 | 4.11 |
| 1449153_PM_at | matrix metallopeptidase 12 | Mmp12 | 17381 | 5.16 | 0.00 | 10.57 |

100MMF-vs-Veh, 12 h

| | | | | | | |
|---|---|---|---|---|---|---|
| 1416273_PM_at | tumor necrosis factor, alpha-induced | Tnfaip2 | 21928 | 1.54 | 0.00 | 2.29 |
| | protein 2 | | | | | |
| 1416298_PM_at | matrix metallopeptidase 9 | Mmp9 | 17395 | 1.82 | 0.00 | 3.64 |
| 1418499_PM_a_at | potassium voltage-gated channel, Isk- | Kcne3 | 57442 | 1.56 | 0.00 | 0.10 |
| | related subfamily, gene 3 | | | | | |
| 1419561_PM_at | chemokine (C-C motif) ligand 3 | Ccl3 | 20302 | 2.22 | 0.00 | 1.07 |
| 1420804_PM_s_at | C-type lectin domain family 4, | Clec4d | 17474 | 2.43 | 0.00 | 5.94 |
| | member d | | | | | |
| 1421037_PM_at | neuronal PAS domain protein 2 | Npas2 | 18143 | 1.56 | 0.00 | 2.25 |
| 1449153_PM_at | matrix metallopeptidase 12 | Mmp12 | 17381 | 3.95 | 0.00 | 1.84 |

100DMF-vs-90MMF, 7 h

| | | | | | | |
|---|---|---|---|---|---|---|
| 1426188_PM_s_at | cDNA sequence BC005685 | BC005685 | 352945 | −1.54 | 0.00 | 4.77 |
| 1428776_PM_at | solute carrier family 10 | Slc10a6 | 75750 | −1.52 | 0.00 | 0.70 |
| | (sodium/bile acid | | | | | |
| | cotransporter family), member 6 | | | | | |

100DMF-vs-90MMF, 12 h

| | | | | | | |
|---|---|---|---|---|---|---|
| 1417130_PM_s_at | angiopoietin-like 4 | Angptl4 | 57875 | 2.93 | 0.00 | 1.91 |
| 1419874_PM_x_at | zinc finger and BTB domain | Zbtb16 | 235320 | 2.14 | 0.00 | 2.87 |
| | containing 16 | | | | | |
| 1437241_PM_at | Kruppel-like factor 11 | Klf11 | 194655 | 1.91 | 0.00 | 6.24 |
| 1442025_PM_a_at | — | — | — | 2.64 | 0.00 | 4.11 |
| 1442026_PM_at | — | — | — | 2.92 | 0.00 | 5.24 |
| 1460409_PM_at | carnitine palmitoyltransferase | Cpt1a | 12894 | 1.56 | 0.00 | 1.09 |
| | 1a, liver | | | | | |

### EQUIVALENTS

Those skilled in the art will recognize, or be able to ascertain using no more than routine experimentation, many equivalents to the specific embodiments of the invention described herein. Such equivalents are intended to be encompassed.

What is claimed is:

1. A method of evaluating, monitoring, stratifying, or treating, a subject, comprising:
a) acquiring a value for the expression of a gene, wherein said gene is chosen from one, two, three, four, five, six or all of the dimethyl fumarate (DMF)-differentially expressed gene from Table 9;
b) responsive to said value,
i) classifying said subject,
ii) selecting said subject for treatment with DMF, or with a treatment other than
DMF, or
iii) administering DMF, or a treatment other than DMF, to said subject,
provided that the method comprises one of treating the subject, directly acquiring the value, or directly acquiring a sample from which the value is acquired.

2. A method of evaluating, or monitoring, an MS treatment, e.g., an MS treatment with a DMF, in a subject having MS, or at risk for developing MS, said method comprising:
administering DMF to the subject;
acquiring from said subject a value for the expression of a gene, wherein said gene is chosen from one, two, three, four, five, six or all of the DMF-differentially expressed gene from Table 9,
wherein a change in the gene expression is indicative of a differential response to DMF.

3. The method of claim 2, further comprising, responsive to said value, treating, selecting and/or altering one or more of: the course of the MS treatment, the dosing of the MS treatment, the schedule or time course of the MS treatment, or administration of a treatment other than DMF.

4. A method of treating a subject having, or at risk of having, MS, said method comprising:
a) administering DMF to the subject in an amount sufficient to treat MS, provided that the subject is identified for treatment with the DMF on the basis of a value for the expression of a gene, wherein said gene is chosen from one, two, three, four, five, six or all of the DMF-differentially expressed gene from Table 9.

5. The method of claim 1, wherein the subject, e.g., a human subject, has an autoimmune disorder, e.g., MS.

6. The method of any of any one of claims 1-5, wherein the subject with MS has a relapsing form of MS.

7. The method of any one of claims 1-6, wherein the subject has been administered DMF, e.g., prior to, or at the time of, acquiring the value.

8. The method of any one of claims 1-7, wherein a tissue of the subject, e.g., the peripheral blood, comprises, greater than background levels, e.g., therapeutic levels, of DMF, MMF, or both, e.g., prior to, or at the time of, acquiring the value.

9. The method of any one of claims 1-8, comprising administering DMF to said subject.

10. The method of any one of claims 1-8, wherein the value for expression of the gene comprises a value for a transcriptional parameter, e.g., the level of an mRNA encoded by the gene.

11. The method of any one of claims 1-8, wherein value for expression of the gene comprises a value for a translational parameter, e.g., the level of a protein encoded by the gene.

12. The method of claim 1, wherein step a) comprises acquiring a value for the expression of a plurality, e.g., 2, 3, 4, 5, 6, or more, genes, and in step b), responsive comprises responsive to one, some, or all, of the acquired values from step a).

13. The method of claim 1, wherein the value for expression of the gene is for blood, e.g., whole blood.

14. The method of any one of claims 1-13, wherein the gene is chosen from one or more of: Granzyme A (Gzma), Natural cytotoxicity triggering receptor 1 (Ncr1), Killer cell lectin-like receptor subfamily C member 1 (Klrc1), Killer cell lectin-like receptor subfamily B member 1B (Klrb1b), or Killer cell lectin-like receptor family E member 1 (Klre1).

15. The method of any one of claims 1-13, wherein the value for expression of the gene comprises a value for a translational parameter, e.g., the level of a protein encoded by the gene, in blood, e.g., whole blood.

16. The method of claim 15, wherein the gene is selected from one or more of Klrc1, Klrb1b, Klrk1, and Klrd1.

17. The method of any one of claims 1-16, wherein the value for expression of the gene is for a blood sample, or a blood derived sample, e.g., serum, or an NK-cell containing fraction, from the subject.

18. The method of any one of claims 1-16, wherein the sample is blood, and comprises, greater than background levels, e.g., therapeutic levels, of DMF, MMF, or both.

19. A device comprising:
one, or a plurality of, e.g., 2, 3, 4, 5, 6, or more, probes, each probe being specific for a product, e.g., a translational product or transcriptional product, of a dimethyl fumarate (DMF)-differentially expressed gene from Table 9,
wherein the device includes less than 10, 25, 50, 100, 200, 250, 300, or 500 probes specific for products, e.g., a translational product or transcriptional product, of genes that are not a dimethyl fumarate (DMF)-differentially expressed gene.

20. The device of claim 19, further comprising, a sample from a tissue of a subject, e.g., the peripheral blood, which comprises greater than background levels, e.g., therapeutic levels, of DMF, MMF, or both.

21. A method of using a device described herein comprising:
providing a device of claim 19;
contacting the device with the sample from a tissue of a subject, e.g., the peripheral blood, which comprises greater than background levels, e.g., therapeutic levels, of DMF, MMF, or both,
thereby using the device.

22. A reaction mixture comprising:
a sample from a tissue of a subject, e.g., the peripheral blood, which comprises greater than background levels, e.g., therapeutic levels, of DMF, MMF, or both;
one, or a plurality of, e.g., 2, 3, 4, 5, 6, or more, probes, each probe being specific for a product, e.g., a translational product or transcriptional product, of a dimethyl fumarate (DMF)-differentially expressed gene from Table 9,
wherein the reaction mixture includes less than 10, 25, 50, 100, 200, 250, 300, or 500 probes specific for products, e.g., a translational product or transcriptional product, of genes that are not a dimethyl fumarate (DMF)-differentially expressed gene.

23. A method of making a reaction mixture comprising:
providing a sample from a tissue of a subject, e.g., the peripheral blood, which comprises greater than background levels, e.g., therapeutic levels, of DMF, MMF, or both;
contacting the sample with one or a plurality of probes described herein, or with a device described herein, one, or a plurality of, e.g., 2, 3, 4, 5, 6, or more, probes, each probe being specific for a product, e.g., a translational product or transcriptional product, of a dimethyl fumarate (DMF)-differentially expressed gene from Table 9,
wherein the reaction mixture includes less than 10, 25, 50, 100, 200, 250, 300, or 500 probes specific for products, e.g., a translational product or transcriptional product, of genes that are not a dimethyl fumarate (DMF)-differentially expressed gene.
thereby making a reaction mixture.

24. A method of treating a subject having a natural killer (NK) function related disorder or condition comprising: administering to the subject in need of treatment a dialkyl fumarate in an amount sufficient to treat the disorder,
wherein the disorder or condition is selected from:
cancer, a viral infection, and inflammation.

25. The method of claim 24, wherein the dialkyl fumarate is: 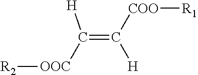
wherein R₁ and R₂, which may be the same or different, independently represent a linear, branched or cyclic, saturated or unsaturated C₁₋₂₀ alkyl radical which may be optionally substituted with halogen (Cl, F, I, Br), hydroxy, C₁₋₄ alkoxy, nitro or cyano.

26. The method of claim 25, wherein R₁ and R₂, which may be the same or different, independently are methyl, ethyl, n-propyl, isopropyl, n-butyl, sec-butyl, t-butyl, pentyl, cyclopentyl, 2-ethyl hexyl, hexyl, cyclohexyl, heptyl, cycloheptyl, octyl, vinyl, allyl, 2-hydroxy ethyl, 2 or 3-hydroxy propyl, 2-methoxy ethyl, methoxy methyl or 2- or 3-methoxy propyl.

27. The method of claim 25, wherein R₁ and R₂ are identical and are methyl or ethyl.

28. The method of claim 25, wherein R₁ and R₂ are methyl.

29. The method of claim 24 or 25, wherein the disorder or condition is cancer.

30. The method of claim 24 or 25, wherein the disorder or condition is a hematological malignancy.

31. The method of claim 30, wherein the hematological malignancy is selected from lymphocytic leukemia, chronic lymphocytic leukemia, and lymphoma.

32. The method of claim 24 or 25, wherein the disorder or condition is a solid tumor.

33. The method of claim 32, wherein the solid tumor is selected from gastrointestinal sarcoma, neuroblastoma, and kidney cancer.

34. The method of claim 24 or 25, wherein the disorder or condition is a viral infection.

35. A method of evaluating, monitoring, stratifying, or treating, a subject, comprising:
a) acquiring a value for the expression of a gene, wherein said gene is chosen from one, two, three, four, five, six, seven, eight or all of FCGR1A, ST18, CCL3L1, VCAM1, CCR3, Klrb1c, Ncr1, DEPP, or Zbtb16;
b) responsive to said value,
i) classifying said subject,
ii) selecting said subject for treatment with DMF, or with a treatment other than
DMF, or
iii) administering DMF, or a treatment other than DMF, to said subject,
provided that the method comprises one of treating the subject, directly acquiring the value, or directly acquiring a sample from which the value is acquired.

36. A method of evaluating, or monitoring, an MS treatment, e.g., an MS treatment with a DMF, in a subject having MS, or at risk for developing MS, said method comprising:
administering DMF to the subject;
acquiring from said subject a value for the expression of a gene, wherein said gene is chosen from one, two, three, four, five, six, seven, eight or all of FCGR1A, ST18, CCL3L1, VCAM1, CCR3, Klrb1c, Ncr1, DEPP, or Zbtb16,
wherein a change in the gene expression is indicative of a differential response to DMF.

37. The method of claim 35 or 36, wherein the method comprises acquiring a value for the expression of FCGR1A.

38. The method of any one of claims 35-37, wherein the method comprises acquiring a value for the expression of ST18.

39. The method of any one of claims 35-38, wherein the method comprises acquiring a value for the expression of CCL3L1.

40. The method of any one of claims 35-39, wherein the method comprises acquiring a value for the expression of VCAM1.

41. The method of any one of claims 35-40, wherein the method comprises acquiring a value for the expression of, CCR3.

42. The method of any one of claims 35-41, wherein the method comprises acquiring a value for the expression of Klrb1c.

43. The method of any one of claims 35-42, wherein the method comprises acquiring a value for the expression of Ncr1.

44. The method of any one of claims 35-43, wherein the method comprises acquiring a value for the expression of DEPP.

45. The method of any one of claims 35-44, wherein the method comprises acquiring a value for the expression of Zbtb16.

46. The method of any one of claims 36-45, further comprising, responsive to said value, treating, selecting and/or altering one or more of: the course of the MS treatment, the dosing of the MS treatment, the schedule or time course of the MS treatment, or administration of a treatment other than DMF.

47. A method of treating a subject having, or at risk of having, MS, said method comprising:
a) administering DMF to the subject in an amount sufficient to treat MS, provided that the subject is identified for treatment with the DMF on the basis of a value for the expression of a gene, wherein said gene is chosen from one, two, three, four, five, six, seven, eight or all of FCGR1A, ST18, CCL3L1, VCAM1, CCR3, Klrb1c, Ncr1, DEPP, or Zbtb16.

48. The method of any one of claims 35-47, wherein the subject, e.g., a human subject, has an autoimmune disorder, e.g., MS.

49. The method of any one of claims 35-48, wherein the subject with MS has a relapsing form of MS.

50. The method of any one of claims 35-49, wherein the subject has been administered DMF, e.g., prior to, or at the time of, acquiring the value.

51. The method of any one of claims 35-50, wherein a tissue of the subject, e.g., the peripheral blood, comprises, greater than background levels, e.g., therapeutic levels, of DMF, MMF, or both, e.g., prior to, or at the time of, acquiring the value.

52. The method of any one of claims 35-51, comprising administering DMF to said subject.

53. The method of any one of claims 35-51, wherein the value for expression of the gene comprises a value for a transcriptional parameter, e.g., the level of an mRNA encoded by the gene.

54. The method of any one of claims 35-51, wherein value for expression of the gene comprises a value for a translational parameter, e.g., the level of a protein encoded by the gene.

55. The method of claim 35, wherein step a) comprises acquiring a value for the expression of a plurality, e.g., 2, 3, 4, 5, 6, or more, genes, and in step b), responsive comprises responsive to one, some, or all, of the acquired values from step a).

56. The method of claim 35, wherein the value for expression of the gene is for blood, e.g., whole blood.

57. The method of any one of claims 35-56, wherein the value for expression of the gene comprises a value for a translational parameter, e.g., the level of a protein encoded by the gene, in blood, e.g., whole blood.

58. The method of any one of claims 35-57, wherein the value for expression of the gene is for a blood sample, or a blood derived sample, e.g., serum, or an NK-cell containing fraction, from the subject.

59. The method of any one of claims 35-57, wherein the sample is blood, and comprises, greater than background levels, e.g., therapeutic levels, of DMF, MMF, or both.

60. A device comprising:
one, or a plurality of, e.g., 2, 3, 4, 5, 6, or more, probes, each probe being specific for a product, e.g., a translational product or transcriptional product, of a dimethyl fumarate (DMF)-differentially expressed gene selected from Appendix B, Appendix C, Appendix D, or Appendix E,
wherein the device includes less than 10, 25, 50, 100, 200, 250, 300, or 500 probes specific for products, e.g., a translational product or transcriptional product, of genes that are not a dimethyl fumarate (DMF)-differentially expressed gene.

61. The device of claim 60, further comprising, a sample from a tissue of a subject, e.g., the peripheral blood, which comprises greater than background levels, e.g., therapeutic levels, of DMF, MMF, or both.

62. A method of using a device described herein comprising:
providing a device of claim 60;
contacting the device with the sample from a tissue of a subject, e.g., the peripheral blood, which comprises greater than background levels, e.g., therapeutic levels, of DMF, MMF, or both,
thereby using the device.

63. A reaction mixture comprising:
a sample from a tissue of a subject, e.g., the peripheral blood, which comprises greater than background levels, e.g., therapeutic levels, of DMF, MMF, or both;
one, or a plurality of, e.g., 2, 3, 4, 5, 6, or more, probes, each probe being specific for a product, e.g., a translational product or transcriptional product, of a dimethyl fumarate (DMF)-differentially expressed gene from Appendix B, Appendix C, Appendix D, or Appendix E,
wherein the reaction mixture includes less than 10, 25, 50, 100, 200, 250, 300, or 500 probes specific for products, e.g., a translational product or transcriptional product, of genes that are not a dimethyl fumarate (DMF)-differentially expressed gene.

64. A method of making a reaction mixture comprising:
providing a sample from a tissue of a subject, e.g., the peripheral blood, which comprises greater than background levels, e.g., therapeutic levels, of DMF, MMF, or both;
contacting the sample with one or a plurality of probes described herein, or with a device described herein, one, or a plurality of, e.g., 2, 3, 4, 5, 6, or more, probes, each probe being specific for a product, e.g., a translational product or transcriptional product, of a dimethyl fumarate (DMF)-differentially expressed gene from Appendix B, Appendix C, Appendix D, or Appendix E,
wherein the reaction mixture includes less than 10, 25, 50, 100, 200, 250, 300, or 500 probes specific for products, e.g., a translational product or transcriptional product, of genes that are not a dimethyl fumarate (DMF)-differentially expressed gene.
thereby making a reaction mixture.
